(19) United States
(12) Patent Application Publication
(10) Pub. No.: US 2023/0149550 A1
(43) Pub. Date: May 18, 2023

(54) METHODS AND COMPOUNDS FOR THE TREATMENT OF GENETIC DISEASE

(71) Applicant: Design Therapeutics, Inc., Carlsbad, CA (US)

(72) Inventors: Aseem ANSARI, Madison, WI (US); Sean J. JEFFRIES, Carlsbad, CA (US); Pratik SHAH, Carlsbad, CA (US); Chengzhi ZHANG, Carlsbad, CA (US)

(21) Appl. No.: 17/759,944

(22) PCT Filed: Feb. 3, 2021
(86) PCT No.: PCT/US2021/016481

§ 371 Date: Aug. 2, 2022

Related U.S. Application Data
(60) Provisional application No. 63/135,476, filed on Jan. 8, 2021.
(60) Provisional application No. 62/969,644, filed on Feb. 3, 2020.

(51) Int. Cl. A61K 47/55; A61K 47/54; A61P 25/28; A61K 47/60

(52) U.S. Cl. CPC A61K 47/55; A61K 47/545; A61P 25/28; A61K 47/60

(57) ABSTRACT

The present disclosure relates to compounds and methods for modulating the expression of dmpk, and treating diseases and conditions in which dmpk plays an active role. The compound can be a transcription modulator molecule having a first terminus, a second terminus, and oligomeric backbone, wherein: a) the first terminus comprises a DNA-binding moiety capable of noncovalently binding to a nucleotide repeat sequence CAG or CTG; b) the second terminus comprises a protein-binding moiety binding to a regulatory molecule that modulates an expression of a gene comprising the nucleotide repeat sequence CAG or CTG; and c) the oligomeric backbone comprising a linker between the first terminus and the second terminus.

### CROSS REFERENCE

This application claims the benefit of U.S. Application No. 62/969,644, filed Feb. 3, 2020 and U.S. Application No. 63/135,476, filed Jan. 8, 2021 which are hereby incorporated by reference in their entirety.

### FIELD OF THE DISCLOSURE

Disclosed herein are new chimeric heterocyclic polyamide compounds and compositions and their application as pharmaceuticals for the treatment of disease. Methods to modulate the expression of fxn in a human or animal subject are also provided for the treatment diseases such as Friedreich's ataxia.

### BACKGROUND OF THE DISCLOSURE

The disclosure relates to the treatment of inherited genetic diseases characterized by overproduction of mRNA.

Friedreich's ataxia (FA or FRDA) is an autosomal recessive neurodegenerative disorder caused by mutations in the fxn gene, which encodes the protein frataxin (FXN), an iron-binding mitochondrial protein involved in electron transport and metabolism. In most subjects with FA, a GAA trinucleotide repeat (from about 66 to over 1000 trinucleotides) is included in the first intron of fxn, and this hyperexpansion is responsible for the observed pathology. Hyperexpansion of the GAA repeats results in reduced expression of FXN.

Friedreich's ataxia is characterized by progressive degradation of the nervous system, particularly sensory neurons. In addition, cardiomyocytes and pancreatic beta cells are susceptible to frataxin depletion. Symptoms usually present by age 18; however, later diagnoses of FA are not uncommon. FA patients develop neurodegeneration of the large sensory neurons and spinocerebellar tracts, as well as cardiomyopathy and diabetes mellitus. Clinical symptoms of FA include ataxia, gait ataxia, muscle weakness, loss of upper body strength, loss of balance, lack of reflexes in lower limbs and tendons, loss of sensation, particularly to vibrations, impairment of position sense, impaired perception of temperature, touch, and pain, hearing and vision impairment, including distorted color vision and involuntary eye movements, irregular foot configuration, including pes cavus and inversion, hearing impairment, dysarthria, dysphagia, impaired breathing, scoliosis, diabetes, intolerance to glucose and carbohydrates, cardiac dysfunctions including hypertrophic cardiomyopathy, arrhythmia, myocardial fibrosis, and cardiac failure. Currently there is no cure for FA, with medical treatments being limited to surgical intervention for the spine and the heart, as well as therapy to assist with balance and coordination, motion, and speech.

### SUMMARY OF THE DISCLOSURE

This disclosure utilizes regulatory molecules present in cell nuclei that control gene expression. Eukaryotic cells provide several mechanisms for controlling gene replication, transcription, and/or translation. Regulatory molecules that are produced by various biochemical mechanisms within the cell can modulate the various processes involved in the conversion of genetic information to cellular components. Several regulatory molecules are known to modulate the production of mRNA and, if directed to fxn, would modulate the production of fxn mRNA that causes Friedreich's ataxia, and thus reverse the progress of the disease.

The disclosure provides compounds and methods for recruiting a regulatory molecule into close proximity to fxn. The compounds disclosed herein contain: (a) a recruiting moiety that will bind to a regulatory molecule, linked to (b) a DNA binding moiety that will selectively bind to fxn. The compounds will counteract the expression of defective fxn in the following manner:
(1) The DNA binding moiety will bind selectively the characteristic GAA trinucleotide repeat sequence of fxn;
(2) The recruiting moiety, linked to the DNA binding moiety, will thus be held in proximity to fxn;
(3) The recruiting moiety, now in proximity to fxn, will recruit the regulatory molecule into proximity with the gene; and
(4) The regulatory molecule will modulate expression, and therefore counteract the production of defective fxn by direct interaction with the gene.

The mechanism set forth above will provide an effective treatment for Friedreich's ataxia, which is caused by the expression of defective fxn. Correction of the expression of the defective fxn gene thus represents a promising method for the treatment of Friedreich's ataxia.

The disclosure provides recruiting moieties that will bind to regulatory molecules. Small molecule inhibitors of regulatory molecules serve as templates for the design of recruiting moieties, since these inhibitors generally act via noncovalent binding to the regulatory molecules.

The disclosure further provides for DNA binding moieties that will selectively bind to one or more copies of the GAA trinucleotide repeat that is characteristic of the defective fxn gene. Selective binding of the DNA binding moiety to fxn, made possible due to the high GAA count associated with the defective fxn gene, will direct the recruiting moiety into proximity of the gene, and recruit the regulatory molecule into position to up-regulate gene transcription.

The DNA binding moiety will comprise a polyamide segment that will bind selectively to the target GAA sequence. Polyamides have been designed by Dervan and others that can selectively bind to selected DNA sequences. These polyamides sit in the minor groove of double helical DNA and form hydrogen bonding interactions with the Watson-Crick base pairs. Polyamides that selectively bind to particular DNA sequences can be designed by linking monoamide building blocks according to established chemical rules. One building block is provided for each DNA base pair, with each building block binding noncovalently and selectively to one of the DNA base pairs: A/T, T/A, G/C, and C/G. Following this guideline, trinucleotides will bind to molecules with three amide units, i.e. triamides. In general, these polyamides will orient in either direction of a DNA sequence, so that the 5′-GAA-3′ trinucleotide repeat sequence of fxn can be targeted by polyamides selective either for GAA or for AAG. Furthermore, polyamides that bind to the complementary sequence, in this case, TTC or CTT, will also bind to the trinucleotide repeat sequence of fxn and can be employed as well.

In principle, longer DNA sequences can be targeted with higher specificity and/or higher affinity by combining a larger number of monoamide building blocks into longer polyamide chains. Ideally, the binding affinity for a polyamide would simply be equal to the sum of each individual monoamide/DNA base pair interaction. In practice, however, due to the geometric mismatch between the fairly rigid polyamide and DNA structures, longer polyamide sequences do not bind to longer DNA sequences as tightly as would be expected from a simple additive contribution. The geometric mismatch between longer polyamide sequences and longer DNA sequences induces an unfavorable geometric strain that subtracts from the binding affinity that would be otherwise expected.

The disclosure therefore provides DNA moieties that comprise triamides that are connected by flexible spacers. The spacers alleviate the geometric strain that would otherwise decrease binding affinity of a larger polyamide sequence.

Disclosed herein are polyamide compounds that can bind to one or more copies of the trinucleotide repeat sequence GAA, and can modulate the expression of the defective fxn gene. Treatment of a subject with these compounds will counteract the expression of the defective fxn gene, and this can reduce the occurrence, severity, and/or frequency of symptoms associated with Friedreich's ataxia. Certain compounds disclosed herein will provide higher binding affinity and/or selectivity than has been observed previously for this class of compound

Other objects, features and advantages of the block copolymers, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below.

FIG. 1 shows changes in fxn gene expression in Friedreich's ataxia patient cells after treatment with transcription modulator molecules.

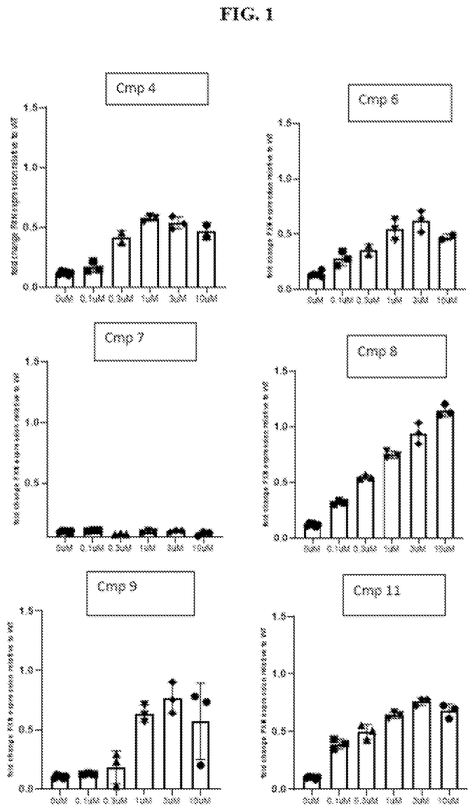

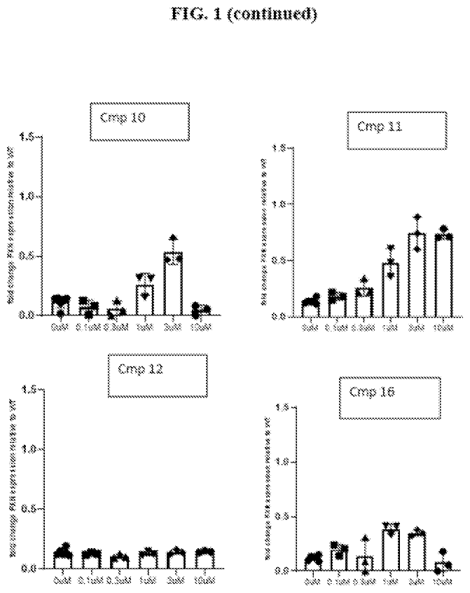

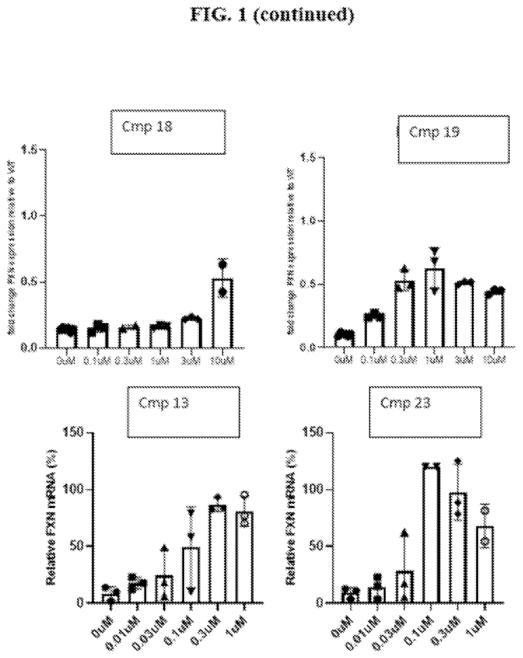

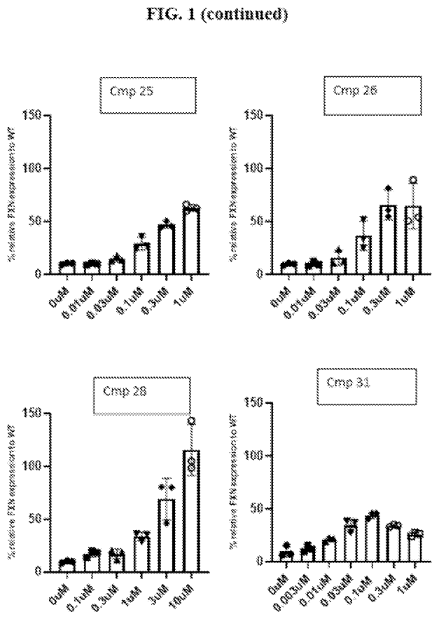

### DETAILED DESCRIPTION OF THE DISCLOSURE

The transcription modulator molecule described herein represents an interface of chemistry, biology and precision medicine in that the molecule can be programmed to regulate the expression of a target gene containing nucleotide repeat GAA. The transcription modulator molecule contains DNA binding moieties that will selectively bind to one or more copies of the GAA hexanucleotide repeat that is characteristic of the defective fxn gene. The transcription modulator molecule also contains moieties that bind to regulatory proteins. The selective binding of the target gene will bring the regulatory protein into proximity to the target gene and thus downregulates transcription of the target gene. The molecules and compounds disclosed herein provide higher binding affinity and selectivity than has been observed previously for this class of compounds and can be more effective in treating diseases associated with the defective fxn gene.

Treatment of a subject with these compounds will modulate the expression of the defective fxn gene, and this can reduce the occurrence, severity, or frequency of symptoms associated with ALS. The transcription modulator molecules described herein recruits the regulatory molecule to modulate the expression of the defective fxn gene and effectively treats and alleviates the symptoms associated with diseases such as Friedreich ataxia.

### Transcription Modulator Molecules

The transcription modulator molecules disclosed herein possess useful activity for modulating the transcription of a target gene having one or more GAA repeats (e.g., fxn), and may be used in the treatment or prophylaxis of a disease or condition in which the target gene (e.g., fxn) plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for modulating the expression of fxn. Other embodiments provide methods for treating a fxn-mediated disorder in a patient in need of such treatment, comprising administering to said patient a therapeutically effective amount of a compound or composition according to the present disclosure. Also provided is the use of certain compounds disclosed herein for use in the manufacture of a medicament for the treatment of a disease or condition ameliorated by the modulation of the expression of fxn.

Some embodiments relate to a transcription modulator molecule or compound having a first terminus, a second terminus, and oligomeric backbone, wherein: a) the first terminus comprises a DNA-binding moiety capable of noncovalently binding to a nucleotide repeat sequence GAA; b) the second terminus comprises a protein-binding moiety binding to a regulatory molecule that modulates an expression of a gene comprising the nucleotide repeat sequence GAA; and c) the oligomeric backbone comprising a linker between the first terminus and the second terminus. In some embodiments, the second terminus is not a Brd4 binding moiety.

In certain embodiments, the compounds have structural Formula (I):

X-L-Y Formula (I)

or a salt thereof, wherein:

X comprises a is a recruiting moiety that is capable of noncovalent binding to a regulatory moiety within the nucleus;

Y comprises a DNA recognition moiety that is capable of noncovalent binding to one or more copies of the trinucleotide repeat sequence GAA; and

L is a linker.

Certain compounds disclosed herein may possess useful activity for modulating the transcription of fxn, and may be used in the treatment and/or prophylaxis of a disease or condition in which fxn plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for modulating the expression of fxn. Other embodiments provide methods for treating a fxn-mediated disorder in a patient in need of such treatment, comprising administering to said patient a therapeutically effective amount of a compound or composition according to the present disclosure. Also provided is the use of certain compounds disclosed herein for use in the manufacture of a medicament for the treatment of a disease or condition ameliorated by the modulation of the expression of fxn.

In certain embodiments, the regulatory molecule is chosen from a bromodomain-containing protein, a nucleosome remodeling factor (NURF), a bromodomain PHD finger transcription factor (BPTF), a ten-eleven translocation enzyme (TET), methylcytosine dioxygenase (TET1), a DNA demethylase, a helicase, an acetyltransferase, and a histone deacetylase (“HDAC”).

In some embodiments, the first terminus is Y, and the second terminus is X, and the oligomeric backbone is L.

In In certain embodiments, the compounds have structural Formula (II):

X-L-(Y₁—Y₂—Y₃)ₙ—Y₀ Formula (II)

or a salt thereof, wherein:

X comprises a recruiting moiety that is capable of noncovalent binding to a regulatory molecule within the nucleus;

L is a linker;

Y₁, Y₂, and Y₃ are internal subunits, each of which comprises a moiety chosen from a heterocyclic ring or a C₁₋₆straight chain aliphatic segment, and each of which is chemically linked to its two neighbors;

Y₀ is an end subunit which comprises a moiety chosen from a heterocyclic ring or a straight chain aliphatic segment, which is chemically linked to its single neighbor;

each subunit can noncovalently bind to an individual nucleotide in the GAA repeat sequence;

n is an integer between 1 and 200, inclusive; and

(Y₁—Y₂—Y₃)ₙ—Y₀ combine to form a DNA recognition moiety that is capable of noncovalent binding to one or more copies of the trinucleotide sequence GAA.

In certain embodiments, the compounds of structural Formula (II) comprise a subunit for each individual nucleotide in the GAA repeat sequence.

In certain embodiment, each internal subunit has an amino (—NH—) group and a carboxy (—CO—) group.

In certain embodiments, the compounds of structural Formula (II) comprise amide (—NHCO—) bonds between each pair of internal subunits.

In certain embodiments, the compounds of structural Formula (II) comprise an amide (—NHCO—) bond between L and the leftmost internal subunit.

In certain embodiments, the compounds of structural Formula (II) comprise an amide bond between the rightmost internal subunit and the end subunit.

In certain embodiments, each subunit comprises a moiety that is independently chosen from a heterocycle and an aliphatic chain.

In certain embodiments, the heterocycle is a monocyclic heterocycle. In certain embodiments, the heterocycle is a monocyclic 5-membered heterocycle. In certain embodiments, each heterocycle contains a heteroatom independently chosen from N, O, or S. In certain embodiments, each heterocycle is independently chosen from pyrrole, imidazole, thiazole, oxazole, thiophene, and furan.

In certain embodiments, the aliphatic chain is a C₁₋₆straight chain aliphatic chain. In certain embodiments, the aliphatic chain has structural formula —(CH₂)ₘ—, for m chosen from 1, 2, 3, 4, and 5. In certain embodiments, the aliphatic chain is —CH₂CH₂—.

In certain embodiments, each subunit comprises a moiety independently chosen from

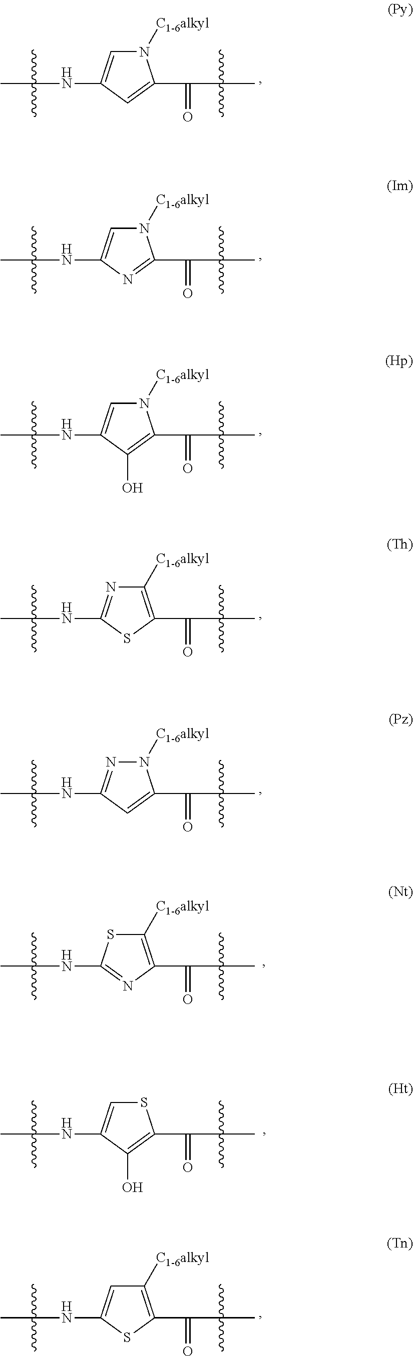

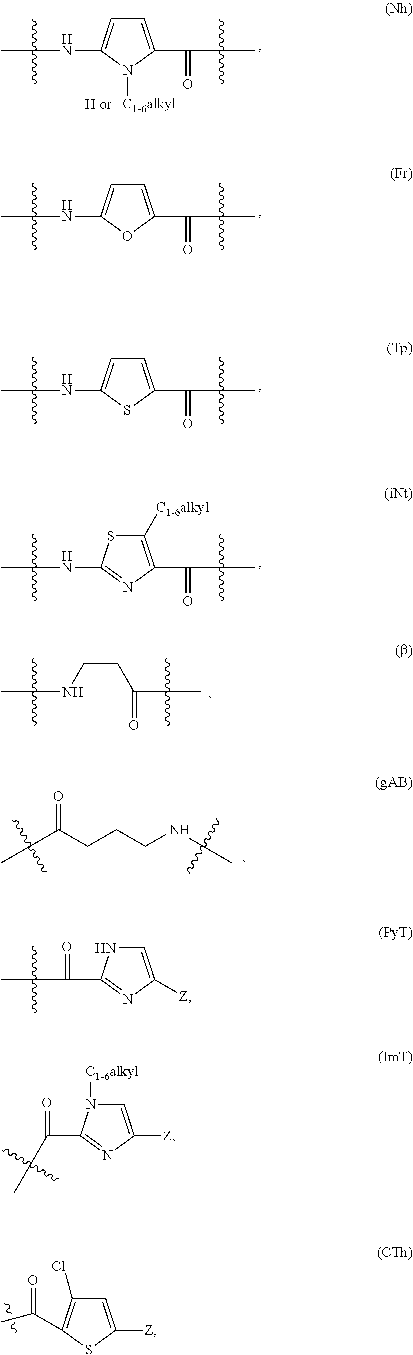

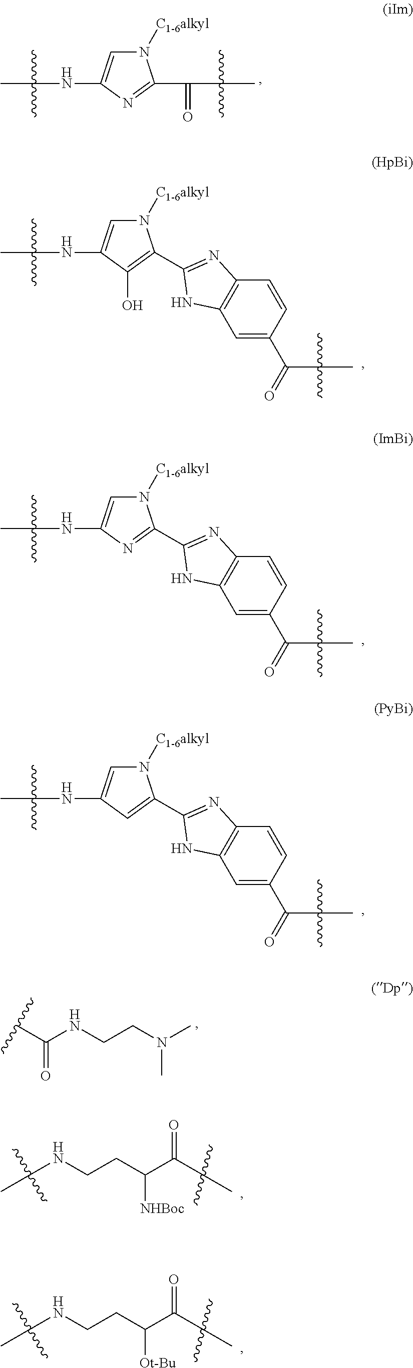

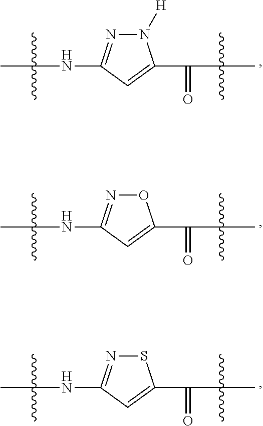 [SMILES: *Nc1cc(C(*)=O)[nH]n1 ; *Nc1cc(C(*)=O)on1 ; *Nc1cc(C(*)=O)sn1]

—NH-benzopyrazinylene-CO—, —NH-phenylene-CO—, —NH-pyridinylene-CO—, —NH-piperidinylene-CO—, —NH-pyrimidinylene-CO—, —NH-anthracenylene-CO—, —NH-quinolinylene-CO—, and

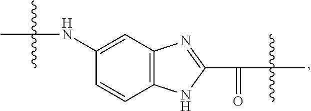

wherein Z is H, NH₂, C₁₋₆ alkyl, C₁₋₆ haloalkyl or C₁₋₆ alkyl-NH₂.

In some embodiments, Py is

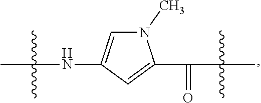 [SMILES: *Nc1cc(C(*)=O)n(C)c1]

### Im is

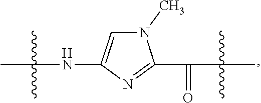 [SMILES: *Nc1cn(C)c(C(*)=O)n1]

### Hp is

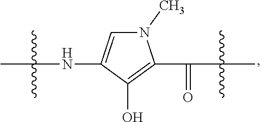 [SMILES: *Nc1cn(C)c(C(*)=O)c1O]

### Th is

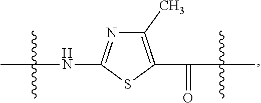 [SMILES: *Nc1nc(C)c(C(*)=O)s1]

### Pz is

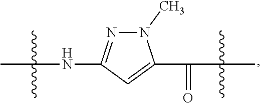 [SMILES: *Nc1cc(C(*)=O)n(C)n1]

### Nt is

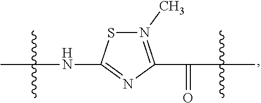

### Tn is

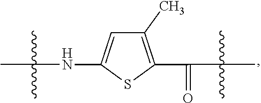 [SMILES: *Nc1cc(C)c(C(*)=O)s1]

### Nh is

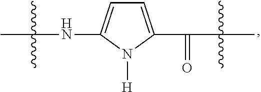 [SMILES: *Nc1ccc(C(*)=O)[nH]1]

iNt is

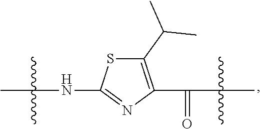 [SMILES: *Nc1nc(C(*)=O)c(C(C)C)s1]

iIm is

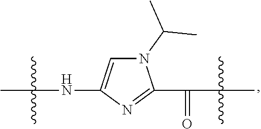 [SMILES: *Nc1cn(C(C)C)c(C(*)=O)n1]

### HpBi is

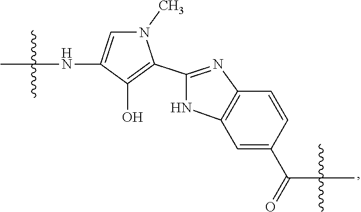 [SMILES: *Nc1cn(C)c(-c2nc3ccc(C(*)=O)cc3[nH]2)c1O]

### ImBi is

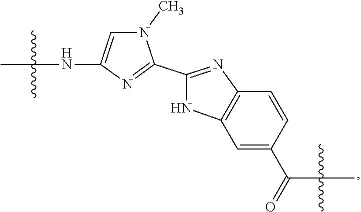 [SMILES: *Nc1cn(C)c(-c2nc3ccc(C(*)=O)cc3[nH]2)n1]

### PyBi is

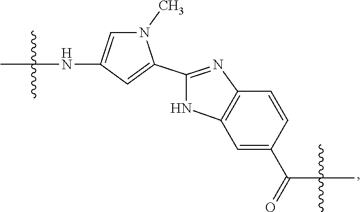 [SMILES: *Nc1cc(-c2nc3ccc(C(*)=O)cc3[nH]2)n(C)c1]

### Dp is

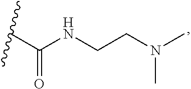 [SMILES: *C(=O)NCCN(C)C]

### —NH-benzopyrazinylene-CO— is

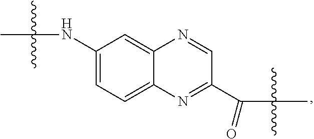 [SMILES: *Nc1ccc2nc(C(*)=O)cnc2c1]

### —NH-phenylene-CO— is

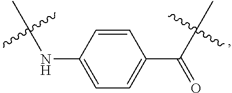 [SMILES: *Nc1ccc(C(*)=O)cc1]

### —NH-pyridinylene-CO— is

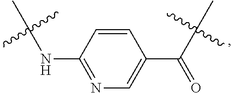 [SMILES: *Nc1ccc(C(*)=O)cn1]

### —NH-piperidinylene-CO— is

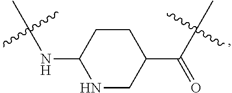 [SMILES: *NC1CCC(C(*)=O)CN1]

### —NH-pyrazinylene-CO— is

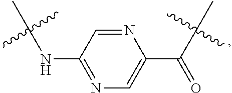 [SMILES: *Nc1cnc(C(*)=O)cn1]

### —NH-anthracenylene-CO— is

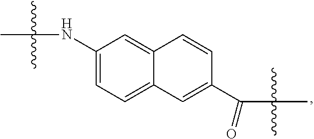 [SMILES: *Nc1ccc2cc(C(*)=O)ccc2c1]

### and —NH-quinolinylene-CO— is

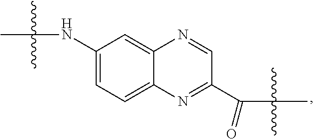 [SMILES: *Nc1ccc2nc(C(*)=O)cnc2c1]

In some embodiments, Py is

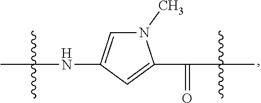 [SMILES: *Nc1cc(C(*)=O)n(C)c1]

### Im is

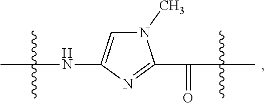 [SMILES: *Nc1cn(C)c(C(*)=O)n1]

### Hp is

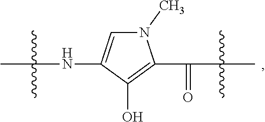 [SMILES: *Nc1cn(C)c(C(*)=O)c1O]

### Th is

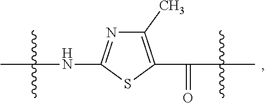 [SMILES: *Nc1nc(C)c(C(*)=O)s1]

### Pz is

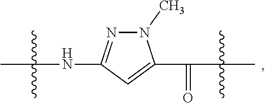 [SMILES: *Nc1cc(C(*)=O)n(C)n1]

### Nt is

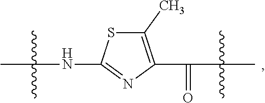 [SMILES: *Nc1nc(C(*)=O)c(C)s1]

### Tn is

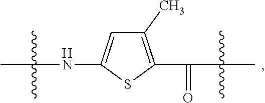 [SMILES: *Nc1cc(C)c(C(*)=O)s1]

### Nh is

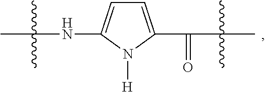 [SMILES: *Nc1ccc(C(*)=O)[nH]1]

iNt is

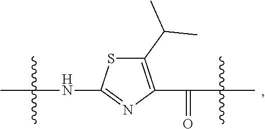 [SMILES: *Nc1nc(C(*)=O)c(C(C)C)s1]

and iIm is

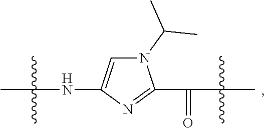 [SMILES: *Nc1cn(C(C)C)c(C(*)=O)n1]

In certain embodiments, n is between 1 and 100, inclusive. n certain embodiments, n is between 1 and 50, inclusive. In certain embodiments, n is between 1 and 20, inclusive. In certain embodiments, n is between 1 and 10, inclusive. In certain embodiments, n is between 1 and 5, inclusive. In certain embodiments, n is an integer between 1 and 3, inclusive. In certain embodiments, n is chosen from 1 and 2. In certain embodiments, n is 1.

In certain embodiments, n is an integer between 1 and 5, inclusive.

In certain embodiments, n is an integer between 1 and 3, inclusive.

In certain embodiments, n is an integer between 1 and 2, inclusive.

In certain embodiments, n is 1.

In certain embodiments, L comprises a C₁₋₆straight chain aliphatic segment.

In certain embodiments, L comprises (CH₂OCH₂)ₘ; and m is an integer between 1 to 20, inclusive. In certain further embodiments, m is an integer between 1 to 10, inclusive. In certain further embodiments, m is an integer between 1 to 5, inclusive.

In certain embodiments, the compounds have structural Formula (III):

X-L-(Y₁—Y₂—Y₃)—(W—Y₁—Y₂—Y₃)ₙ—Y₀ Formula (III)

or a salt thereof, wherein:

X comprises a recruiting moiety that is capable of noncovalent binding to a regulatory molecule within the nucleus;

L is a linker;

Y₁, Y₂, and Y₃ are internal subunits, each of which comprises a moiety chosen from a heterocyclic ring or a C₁₋₆straight chain aliphatic segment, and each of which is chemically linked to its two neighbors;

Y₀ is an end subunit which comprises a moiety chosen from a heterocyclic ring or a straight chain aliphatic segment, which is chemically linked to its single neighbor;

each subunit can noncovalently bind to an individual nucleotide in the GAA repeat sequence;

W is a spacer;

n is an integer between 1 and 200, inclusive; and

(Y₁—Y₂—Y₃)—(W—Y₁—Y₂—Y₃)ₙ—Y₀ combine to form a DNA recognition moiety that is capable of noncovalent binding to one or more copies of the trinucleotide repeat sequence GAA.

In certain embodiments, Y₁—Y₂—Y₃ is:

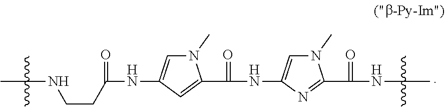 [SMILES: *NCCC(=O)Nc1cc(C(=O)Nc2cn(C)c(C(=O)N*)n2)n(C)c1]

In certain embodiments, Y₁—Y₂—Y₃ is:

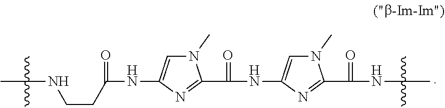 [SMILES: *NCCC(=O)Nc1cn(C)c(C(=O)Nc2cn(C)c(C(=O)N*)n2)n1]

In certain embodiments, Y₁—Y₂—Y₃ is Im-Py-β.

In certain embodiments, Y₁—Y₂—Y₃ is Im-Im-β.

In certain embodiments, each Y₁—Y₂—Y₃ is independently chosen from β-Py-Im and β-Im-Im.

In certain embodiments, at most one Y₁—Y₂—Y₃ is p-Im-Im.

In certain embodiments of the compound of structural Formula (III), n is between 1 and 100, inclusive. In certain embodiments of the compound of structural Formula (III), n is between 1 and 50, inclusive. In certain embodiments of the compound of structural Formula (III), n is between 1 and 20, inclusive. In certain embodiments of the compound of structural Formula (III), n is between 1 and 10, inclusive. In certain embodiments of the compound of structural Formula (III), n is between 1 and 5, inclusive. In certain embodiments of the compound of structural Formula (III), n is chosen from 1 and 2.

In certain embodiments of the compound of structural Formula (III), n is 1.

In certain embodiments, the compounds have structural Formula (IV):

X-L-(Y₁—Y₂—Y₃)—V—(Y₄—Y₅—Y₆)—Y₀ Formula (IV)

or a salt thereof, wherein:

X comprises a recruiting moiety that is capable of noncovalent binding to a regulatory molecule within the nucleus;

Y₁, Y₂, Y₃, Y₄, Y₅, and Y₆ are internal subunits, each of which comprises a moiety chosen from a heterocyclic ring or a C₁₋₆straight chain aliphatic segment, and each of which is chemically linked to its two neighbors;

Y₀ is an end subunit which comprises a moiety chosen from a heterocyclic ring or a straight chain aliphatic segment, which is chemically linked to its single neighbor;

each subunit can noncovalently bind to an individual nucleotide in the GAA repeat sequence;

L is a linker;

V is a turn component for forming a hairpin turn;

n is an integer between 1 and 200, inclusive; and

(Y₁—Y₂—Y₃)—V—(Y₄—Y₅—Y₆)—Y₀ combine to form a DNA recognition moiety that is capable of noncovalent binding to one or more copies of the trinucleotide repeat sequence GAA.

In certain embodiments of the compound of structural Formula (IV), n is between 1 and 100, inclusive. In certain embodiments of the compound of structural Formula (IV), n is between 1 and 50, inclusive. In certain embodiments of the compound of structural Formula (IV), n is between 1 and 20, inclusive. In certain embodiments of the compound of structural Formula (IV), n is between 1 and 10, inclusive. In certain embodiments of the compound of structural Formula (IV), n is between 1 and 5, inclusive. In certain embodiments of the compound of structural Formula (IV), n is chosen from 1 and 2.

In certain embodiments of the compound of structural Formula (IV), n is 1.

In certain embodiments, V is —HN—CH₂CH₂CH₂—CO—.

In certain embodiments, the compounds have structural Formula (V):

X—C(═O)—CH₂CH₂—(Y₁—Y₂—Y₃)ₙ—NH—Y₀ Formula (V)

or a salt thereof, wherein:

X comprises a recruiting moiety that is capable of noncovalent binding to a regulatory molecule within the nucleus;

each Y₁—Y₂—Y₃ is independently chosen from β-Py-Im and β-Im-Im;

Y₀ is an end subunit which comprises a moiety chosen from a heterocyclic ring or a straight chain aliphatic segment, which is chemically linked to its single neighbor; and

n is an integer between 1 and 200, inclusive.

In certain embodiments of the compounds of structural Formula (V), at most one of Y₁—Y₂—Y₃ is p-Im-Im.

In certain embodiments of the compounds of structural Formula (V), Y₁—Y₂—Y₃ is β-Py-Im.

In certain embodiments of the compound of structural Formula (V), n is between 1 and 100, inclusive. In certain embodiments of the compound of structural Formula (V), n is between 1 and 50, inclusive. In certain embodiments of the compound of structural Formula (V), n is between 1 and 20, inclusive. In certain embodiments of the compound of structural Formula (V), n is between 1 and 10, inclusive. In certain embodiments of the compound of structural Formula (V), n is between 1 and 5, inclusive. In certain embodiments of the compound of structural Formula (V), n is chosen from 1 and 2. In certain embodiments of the compound of structural Formula (V), n is 1.

In certain embodiments, the compounds have structural Formula (VI):

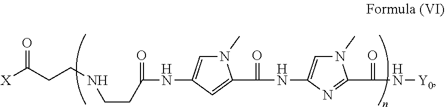

or a salt thereof, wherein:

X comprises a recruiting moiety that is capable of noncovalent binding to a regulatory molecule within the nucleus;

Y₀ is an end subunit which comprises a moiety chosen from a heterocyclic ring or a straight chain aliphatic segment, which is chemically linked to its single neighbor; and

n is an integer between 1 and 200, inclusive.

In certain embodiments of the compound of structural Formula (VI), n is between 1 and 100, inclusive. In certain embodiments of the compound of structural Formula (VI), n is between 1 and 50, inclusive. In certain embodiments of the compound of structural Formula (VI), n is between 1 and 20, inclusive. In certain embodiments of the compound of structural Formula (VI), n is between 1 and 10, inclusive. In certain embodiments of the compound of structural Formula (VI), n is between 1 and 5, inclusive. In certain embodiments of the compound of structural Formula (VI), n is chosen from 1 and 2. In certain embodiments of the compound of structural Formula (VI), n is 1.

In certain embodiments, the compounds have structural Formula (VII):

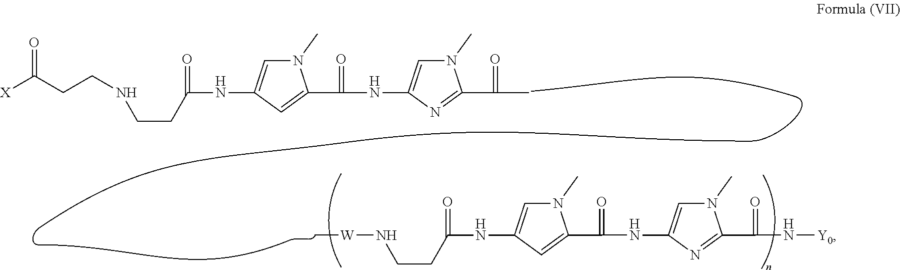

or a salt thereof, wherein:

X comprises a recruiting moiety that is capable of noncovalent binding to a regulatory molecule within the nucleus; and

W is a spacer;

Y₀ is an end subunit which comprises a moiety chosen from a heterocyclic ring or a straight chain aliphatic segment, which is chemically linked to its single neighbor; and

n is an integer between 1 and 200, inclusive.

In certain embodiments of the compound of structural Formula (VII), n is between 1 and 100, inclusive. In certain embodiments of the compound of structural Formula (VII), n is between 1 and 50, inclusive. In certain embodiments of the compound of structural Formula (VII), n is between 1 and 20, inclusive. In certain embodiments of the compound of structural Formula (VII), n is between 1 and 10, inclusive. In certain embodiments of the compound of structural Formula (VII), n is between 1 and 5, inclusive. In certain embodiments of the compound of structural Formula (VII), n is chosen from 1 and 2. In certain embodiments of the compound of structural Formula (VII), n is 1.

In certain embodiments of the compounds of structural Formula (VII), wherein: W is —NHCH₂—(CH₂OCH₂)ₚ—CH₂CO—; and p is an integer between 1 and 4, inclusive.

In some embodiments, (V) is —(CH₂)ₐ—NR¹—(CH₂)_{b}—, —(CH₂)ₐ—, —(CH₂)ₐ—O—(CH₂)_{b}—, —(CH₂)ₐ—CH(NHR¹)—, —(CH₂)ₐ—CH(NHR¹)—, —(CR²R³)ₐ, or —(CH₂)ₐ—CH(NR¹₃)⁺—(CH₂)_{b}—, wherein each a is independently an integer between 2 and 4; R¹ is H, an optionally substituted C₁₋₆ alkyl, an optionally substituted C₃₋₁₀ cycloalkyl, an optionally substituted C₆₋₁₀ aryl, an optionally substituted 4-10 membered heterocyclyl, or an optionally substituted 5-10 membered heteroaryl; each R² and R³ are independently H, halogen, OH, NHAc, or C₁₋₄ alky. In some embodiments, R¹ is H. In some embodiments, R¹ is C₁₋₆ alkyl optionally substituted by 1-3 substituents selected from —C(O)-phenyl. In some embodiments, (V) is —(CR²R³)—(CH₂)ₐ— or —(CH₂)ₐ—(CR²R³)—(CH₂)_{b}—, wherein each a is independently 1-3, b is 0-3, and each R² and R³ are independently H, halogen, OH, NHAc, or C₁₋₄ alky. In some embodiments, (V) is —(CH₂)—CH(NH₃)⁺—(CH₂)— or —(CH₂)—CH₂CH(NH₃)—.

In one aspect, the compounds of the present disclosure bind to the GAA of fxn and recruit a regulatory moiety to the vicinity of fxn. The regulatory moiety, due to its proximity to the gene, will be more likely to modulate the expression of fxn.

Also provided are embodiments wherein any compound disclosed above, including compounds of Formulas (I)-(VIII), are singly, partially, or fully deuterated. Methods for accomplishing deuterium exchange for hydrogen are known in the art.

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

In one aspect, the compounds of the present disclosure bind to the GAA of fxn and recruit a regulatory moiety to the vicinity of fxn. The regulatory moiety, due to its proximity to the gene, will be more likely to modulate the expression of fxn.

In one aspect, the compounds of the present disclosure provide a polyamide sequence for interaction of a single polyamide subunit to each base pair in the GAA repeat sequence. In one aspect, the compounds of the present disclosure provide a turn component V, in order to enable hairpin binding of the compound to the GAA, in which each nucleotide pair interacts with two subunits of the polyamide.

In one aspect, the compounds of the present disclosure provide more than one copy of the polyamide sequence for noncovalent binding to the fxn, and the individual polyamide sequences in this compound are linked by a spacer W, as defined above. The spacer W allows this compound to adjust its geometry as needed to alleviate the geometric strain that otherwise affects the noncovalent binding of longer polyamide sequences.

### First Terminus DNA Binding Moiety

The first terminus interacts and binds with the gene, particularly with the minor grooves of the GAA sequence. In one aspect, the compounds of the present disclosure provide a polyamide sequence for interaction of a single polyamide subunit to each base pair in the GAA repeat sequence. In one aspect, the compounds of the present disclosure provide a turn component (e.g., aliphatic amino acid moiety), in order to enable hairpin binding of the compound to the GAA, in which each nucleotide pair interacts with two subunits of the polyamide.

In one aspect, the compounds of the present disclosure are more likely to bind to the repeated GAA of fxn than to GAA elsewhere in the subject's DNA, due to the high number of GAA repeats associated with fxn.

In one aspect, the compounds of the present disclosure provide more than one copy of the polyamide sequence for noncovalent binding to GAA. In one aspect, the compounds of the present disclosure bind to fxn with an affinity that is greater than a corresponding compound that contains a single polyamide sequence.

In one aspect, the compounds of the present disclosure provide more than one copy of the polyamide sequence for noncovalent binding to the GAA, and the individual polyamide sequences in this compound are linked by a spacer W, as defined above. The spacer W allows this compound to adjust its geometry as needed to alleviate the geometric strain that otherwise affects the noncovalent binding of longer polyamide sequences.

In certain embodiments, the DNA recognition or binding moiety binds in the minor groove of DNA.

In certain embodiments, the DNA recognition or binding moiety comprises a polymeric sequence of monomers, wherein each monomer in the polymer selectively binds to a certain DNA base pair.

In certain embodiments, the DNA recognition or binding moiety comprises a polyamide moiety.

In certain embodiments, the DNA recognition or binding moiety comprises a polyamide moiety comprising heteroaromatic monomers, wherein each heteroaromatic monomer binds noncovalently to a specific nucleotide, and each heteroaromatic monomer is attached to its neighbor or neighbors via amide bonds.

In certain embodiments, the DNA recognition moiety binds to a sequence comprising at least 1000 pentanucleotide repeats. In certain embodiments, the DNA recognition moiety binds to a sequence comprising at least 500 trinucleotide repeats. In certain embodiments, the DNA recognition moiety binds to a sequence comprising at least 200 trinucleotide repeats. In certain embodiments, the DNA recognition moiety binds to a sequence comprising at least 100 trinucleotide repeats. In certain embodiments, the DNA recognition moiety binds to a sequence comprising at least 50 trinucleotide repeats. In certain embodiments, the DNA recognition moiety binds to a sequence comprising at least 20 trinucleotide repeats.

In certain embodiments, the compounds comprise a cell-penetrating ligand moiety.

In certain embodiments, the cell-penetrating ligand moiety is a polypeptide.

In certain embodiments, the cell-penetrating ligand moiety is a polypeptide containing fewer than 30 amino acid residues.

In certain embodiments, the polypeptide is chosen from any one of SEQ ID NO. 1 to SEQ ID NO. 37, inclusive.

The form of the polyamide selected can vary based on the target gene. The first terminus can include a polyamide selected from the group consisting of a linear polyamide, a hairpin polyamide, a H-pin polyamide, an overlapped polyamide, a slipped polyamide, a cyclic polyamide, a tandem polyamide, and an extended polyamide. In some embodiments, the first terminus comprises a linear polyamide. In some embodiments, the first terminus comprises a hairpin polyamide.

The binding affinity between the polyamide and the target gene can be adjusted based on the composition of the polyamide. In some embodiments, the polyamide is capable of binding the DNA with an affinity of less than about 600 nM, about 500 nM, about 400 nM, about 300 nM, about 250 nM, about 200 nM, about 150 nM, about 100 nM, or about 50 nM. In some embodiments, the polyamide is capable of binding the DNA with an affinity of less than about 300 nM. In some embodiments, the polyamide is capable of binding the DNA with an affinity of less than about 500 nM. In some embodiments, the polyamide is capable of binding the DNA with an affinity of less than about 300 nM. In some embodiments, the polyamide is capable of binding the DNA with an affinity of less than about 200 nM. In some embodiments, the polyamide is capable of binding the DNA with an affinity of less than about 100 nM. In some embodiments, the polyamide is capable of binding the DNA with an affinity of less than about 50 nM. In some embodiments, the polyamide is capable of binding the DNA with an affinity of greater than about 200 nM, about 150 nM, about 100 nM, about 50 nM, about 10 nM, or about 1 nM. In some embodiments, the polyamide is capable of binding the DNA with an affinity in the range of about 1-600 nM, 10-500 nM, 20-500 nM, 50-400 nM, or 100-300 nM.

The binding affinity between the polyamide and the target DNA can be determined using a quantitative footprint titration experiment. The experiment involve measuring the dissociation constant K_{d} of the polyamide for target sequence at either 24° C. or 37° C., and using either standard polyamide assay solution conditions or approximate intracellular solution conditions.

The binding affinity between the regulatory protein and the ligand on the second terminus can be determined using an assay suitable for the specific protein. The experiment involve measuring the dissociation constant K_{d} of the ligand for protein and using either standard protein assay solution conditions or approximate intracellular solution conditions.

In some embodiments, the first terminus comprises —NH-Q-C(O)—, wherein Q is an optionally substituted C₆₋₁₀ arylene group, optionally substituted 4-10 membered heterocyclene, optionally substituted 5-10 membered heteroarylene group, or an optionally substituted alkylene group. In some embodiments, Q is an optionally substituted C₆₋₁₀ arylene group or optionally substituted 5-10 membered heteroarylene group. In some embodiments, Q is an optionally substituted 5-10 membered heteroarylene group. In some embodiments, the 5-10 membered heteroarylene group is optionally substituted with 1-4 substituents selected from H, OH, halogen, C₁₋₁₀ alkyl, NO₂, CN, NR′R″, C₁₋₆ haloalkyl, C₁₋₆ alkoxyl, C₁₋₆ haloalkoxy, (C₁₋₆ alkoxy)C₁₋₆ alkyl, C₂₋₁₀ alkenyl, C₂₋₁₀ alkynyl, C₃₋₇ carbocyclyl, 4-10 membered heterocyclyl, C₆₋₁₀ aryl, 5-10 membered heteroaryl, (C₃₋₇carbocyclyl)C₁₋₆ alkyl, (4-10 membered heterocyclyl)C₁₋₆ alkyl, (C₆₋₁₀ aryl)C₁₋₆ alkyl, (C₆₋₁₀ aryl)C₁₋₆ alkoxy, (5-10 membered heteroaryl)C₁₋₆ alkyl, (C₃₋₇carbocyclyl)-amine, (4-10 membered heterocyclyl)amine, (C₆₋₁₀aryl)amine, (5-10 membered heteroaryl)amine, acyl, C-carboxy, O-carboxy, C-amido, N-amido, S-sulfonamido, N-sulfonamido, —SR′, COOH, or CONR′R″; wherein each R′ and R″ are independently H, C₁₋₁₀ alkyl, C₁₋₁₀ haloalkyl, C₁₋₁₀ alkoxyl.

In some embodiments, the first terminus comprises —NH-Q-C(O)—, wherein Q is an optionally substituted C₆₋₁₀ arylene, optionally substituted 4-10 membered heterocyclene, optionally substituted 5-10 membered heteroarylene group, or an optionally substituted alkylene group.

In some embodiments, the first terminus comprises at least three aromatic carboxamide moieties selected to correspond to the nucleotide repeat sequence GAA and at least one aliphatic amino acid residue chosen from the group consisting of glycine, β-alanine, γ-aminobutyric acid, 2,4-diaminobutyric acid, and 5-aminovaleric acid. In some embodiments, the first terminus comprises at least one β-alanine subunit. In some embodiments, the first terminus comprises at least three heteroaromatic carboxamide moieties comprising at least one heteroatom selected from O, N, and S, and at least one aliphatic amino acid residue chosen from the group consisting of glycine, β-alanine, γ-aminobutyric acid, 2,4-diaminobutyric acid, and 5-aminovaleric acid.

In some embodiments, the heteroaromatic carboxamide moiety is a monocyclic or bicyclic moiety.

In some embodiments, the monomer element is independently selected from the group consisting of optionally substituted pyrrole carboxamide monomer, optionally substituted imidazole carboxamide monomer, optionally substituted C—C linked heteromonocyclic/heterobicyclic moiety, and β-alanine. the first terminus comprises one or more carboxamide moieties selected from the group consisting of optionally substituted pyrrole carboxamide monomer, optionally substituted imidazole carboxamide monomer, and β-alanine monomer.

In some embodiments, the first terminus comprises a structure of Formula (A-1), or a pharmaceutically acceptable salt thereof:

-L₁ₐ-[A-M]ₚ-E₁ (A-1)

wherein;

each [A-M] appears p times and p is an integer in the range of 1 to 10,

L₁ₐ is a bond, a C₁₋₆ alkylene, —NR^{a}—C₁₋₆ alkylene-C(O)—, —NR^{a}C(O)—, —NR^{a}—C₁₋₆ alkylene, —O—, or —O—C₁₋₆ alkylene;

each A is selected from the group consisting of a bond, C₁₋₁₀ alkylene, optionally substituted C₆₋₁₀ arylene group, optionally substituted 4-10 membered heterocyclene, optionally substituted 5-10 membered heteroarylene group, —C₁₋₁₀ alkylene-C(O)—, —C₁₋₁₀ alkylene-NR^{a}—, —CO—, —NR^{a}—CONR^{a}—, —CONR^{a}C₁₋₄alkylene-, —NR^{a}CO—C₁₋₄alkylene-, —C(O)O—, —O—, —S—, —S(O)—, —S(O)₂—, —C(═S)—NH—, —C(O)—NH—NH—, —C(O)—N═N—, —C(O)—CH═CH—, (CH₂)₀₋₄—CH═CH—(CH₂)₀₋₄, —N(CH₃)—C₁₋₆ alkylene, and

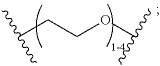

—NH—C₁₋₆ alkylene-NH—, —O—C₁₋₆ alkylene-O—, —NH—N═N—, —NH—C(O)—NH—, and any combinations thereof, and at least one A is —CONH—;

each M is an optionally substituted C₆₋₁₀ arylene group, optionally substituted 4-10 membered heterocyclene, optionally substituted 5-10 membered heteroarylene group, or an optionally substituted alkylene;

E₁ is H or -A^{E}-G;

A^{E} is absent or —NHCO—;

G is selected from the group consisting of optionally substituted C₆₋₁₀ aryl, optionally substituted 4-10 membered heterocyclyl, optionally substituted 5-10 membered heteroaryl, an optionally substituted C₁₋₆ alkyl, C₀₋₄ alkylene-NHC(═NH)NH, —CN, —C₀₋₄alkylene-C(═NH)(NR^{a}R^{b}), —C₀₋₄alkylene-C(═N⁺H₂)(NR^{a}R^{b})C₁₋₅alkylene-NR^{a}R^{b}, C₀₋₄ alkylene-NHC(═NH)R^{a}, and optionally substituted amine; and

each R^{a} and R^{b} are independently selected from the group consisting of H, an optionally substituted C₁₋₆ alkyl, an optionally substituted C₃₋₁₀ cycloalkyl, optionally substituted C₆₋₁₀ aryl, optionally substituted 4-10 membered heterocyclyl, and optionally substituted 5-10 membered heteroaryl.

In some embodiments, the first terminus comprises a polyamide having the structure of Formula (A-2), or a pharmaceutically acceptable salt thereof:

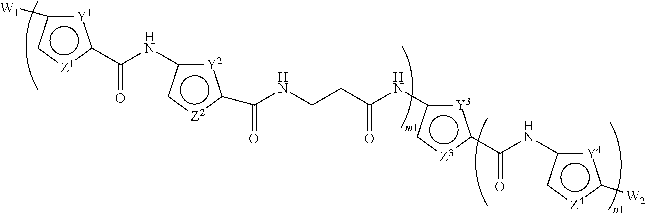

wherein:

m1 is 1-4;

n1 is 0-2;

each Y¹, Y², Y³, and Y⁴ is independently CH or N;

each Z¹, Z², Z³, and Z⁴ is independently O, S, or NR^{1D};

W₁ is hydrogen, an optionally substituted C₁-C₆ alkyl, —NR^{1E}—C(O)—NR^{1E}R^{1F}, —C(O)—NR^{1E}R^{1F}, or (AA)₁₋₁₀;

W₂ is hydrogen, an optionally substitutedC₁-C₆ alkyl, —C(O)—NR^{1E}R^{1F}, or (AA)₁₋₁₀; wherein
AA is an amino acid residue;
each R^{1D} and R^{1E} is independently hydrogen or C₁-C₆ alkyl; and
R^{1F} is hydrogen, an optionally substituted C₁-C₁₀ alkyl, C₁-C₁₀ heteroalkyl, PEG₁₋₂₀, or (AA)₁₋₁₀.

In some embodiments, W₂ is an optionally substituted C₁-C₆ alkyl, —C(O)—NR^{1E}R^{1F}, or (AA)₁₋₁₀. In some embodiments, W² is hydrogen.

In some embodiments, W² is (AA)₁₋₁₀. In some embodiments, AA is an amino acid residue selected from β-alanine, lysine, and arginine. In some embodiments, the AA is a naturally occurring or non-naturally occurring amino acid. In some embodiments, AA is a naturally occurring amino acid. In some embodiments the AA is β-alanine (beta alanine), lysine, or arginine. In some embodiments the AA is at least one β-alanine.

In some embodiments, the first terminus comprises a polyamide having the structure of Formula (A-3), or a pharmaceutically acceptable salt thereof:

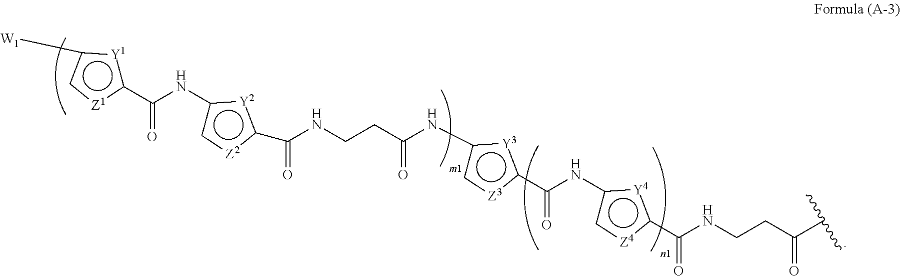

In some embodiments, each Z¹, Z², Z³, and Z⁴ is independently NR^{1D}, wherein R^{1D} is C₁-C₆ alkyl. In some embodiments, each Z¹, Z², Z³, and Z⁴ is independently NCH₃.

In some embodiments, the first terminus comprises a polyamide having the structure of Formula (A-4), or a pharmaceutically acceptable salt thereof:

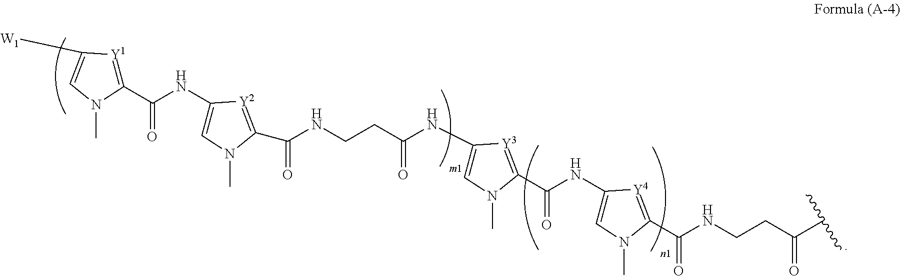

In some embodiments, each Y¹ and Y³ are N; and each Y² and Y⁴ are independently CH or N. In some embodiments, each Y² and Y⁴ is independently CH. In some embodiments, each Y² and Y⁴ is independently N. In some embodiments, Y² is CH and Y⁴ is N. In some embodiments, Y² is N and Y⁴ is CH.

In some embodiments, m1 is 2 or 3; and n1 is 0 or 1.

In some embodiments, m1 is 2. In some embodiments, m1 is 1.

In some embodiments, n1 is 0. In some embodiments, n1 is 1.

In some embodiments, W₁ is optionally substituted C₁-C₆ alkyl, or —C(O)—NR^{1E}R^{1F}. In some embodiments, W₁ is —C(O)—NR^{1E}R^{1F}, wherein R^{1E} is hydrogen; and R^{1F} is hydrogen, optionally substituted C₁-C₁₀ alkyl, or PEG₁₋₂₀.

In some embodiments, W₁ is —C(O)—NR^{1E}R^{1F}, wherein R^{1E} is hydrogen; and R^{1F} is (AA)₁₋₁₀. In some embodiments, AA is an amino acid chain comprising between 1-4, 1-3, or 1-2 amino acids.

In some embodiments, W¹ is (AA)₁₋₁₀. In some embodiments, W¹ is an amino acid chain. In some embodiments, AA comprises an amino acid residue selected from β-alanine, lysine, and arginine. In some embodiments, the AA is a naturally occurring or non-naturally occurring amino acid. In some embodiments, AA is a naturally occurring amino acid. In some embodiments the AA is β-alanine, lysine, or arginine. In some embodiments the AA is β-alanine. In some embodiments, AA is arginine. In some embodiments, AA is lysine. In some embodiments the AA chain length is 1-10, 1-9, 1-8, 1-7, 1-6, 1-5, 1-4, 1-3, or 1-2 amino acids.

In some embodiments, W₁ is hydrogen.

In some embodiments, the first terminus comprises a polyamide having the structure of Formula (A-6), or a pharmaceutically acceptable salt thereof:

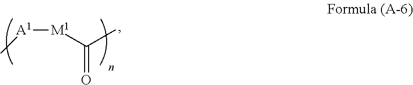

wherein;

each A¹ is —NH— or —NH—(CH₂)ₘ—CH₂—C(O)—NH—;

each M is an optionally substituted C₆₋₁₀ arylene group, optionally substituted 4-10 membered heterocyclene, optionally substituted 5-10 membered heteroarylene group, or optionally substituted alkylene;

m is an integer between 1 to 10; and

n is an integer between 1 and 6.

In some embodiments, each M¹ in [A¹-M¹] of Formula (A-6) is a C₆₋₁₀ arylene group, 4-10 membered heterocyclene, optionally substituted 5-10 membered heteroarylene group, or C₁₋₆ alkylene; each optionally substituted by 1-3 substituents selected from H, OH, halogen, C₁-10 alkyl, NO₂, CN, NR′R″, C₁₋₆ haloalkyl, —C₁₋₆ alkoxyl, C₁₋₆ haloalkoxy, (C₁₋₆ alkoxy)C₁₋₆ alkyl, C₂₋₁₀alkenyl, C₂₋₁₀alkynyl, C₃₋₇ carbocyclyl, 4-10 membered heterocyclyl4-10 membered heterocyclyl, C₆₋₁₀aryl, 5-10 membered heteroaryl, —(C₃₋₇carbocyclyl)C₁₋₆alkyl, (4-10 membered heterocyclyl4-10 membered heterocyclyl)C₁₋₆alkyl, (C₆₋₁₀aryl)C₁₋₆alkyl, (C₆₋₁₀aryl)C₁₋₆alkoxy, (5-10 membered heteroaryl)C₁₋₆alkyl, —(C₃₋₇carbocyclyl)-amine, (4-10 membered heterocyclyl)amine, (C₆₋₁₀aryl)amine, (5-10 membered heteroaryl)amine, acyl, C-carboxy, O-carboxy, C-amido, N-amido, S-sulfonamido, N-sulfonamido, —SR, COOH, or CONR′R″; wherein each R′ and R″ are independently H, C₁₋₁₀ alkyl, C₁₋₁₀ haloalkyl, —C₁₋₁₀ alkoxyl. In some embodiments, each R¹ in [A¹-R¹] of Formula (A-6) is a 5-10 membered heteroarylene containing at least one heteroatoms selected from O, S, and N or a C₁₋₆ alkylene, and the heteroarylene or the a C₁₋₆ alkylene is optionally substituted with 1-3 substituents selected from OH, halogen, C₁₋₁₀ alkyl, NO₂, CN, NR′R″, C₁₋₆ haloalkyl, —C₁₋₆ alkoxyl, C₁₋₆ haloalkoxy, C₃₋₇ carbocyclyl, 4-10 membered heterocyclyl, C₆₋₁₀aryl, 5-10 membered heteroaryl, —SR, COOH, or CONR′R″; wherein each R′ and R″ are independently H, C₁₋₁₀ alkyl, C₁₋₁₀ haloalkyl, —C₁₋₁₀ alkoxyl. In some embodiments, each R¹ in [A¹-R¹] of Formula (A-6) is a 5-10 membered heteroarylene containing at least one heteroatoms selected from O, S, and N, and the heteroarylene is optionally substituted with 1-3 substituents selected from OH, C₁₋₆ alkyl, halogen, and C₁₋₆ alkoxyl.

In some embodiments, the first terminus has a structure of Formula (A-7), or a pharmaceutically acceptable salt thereof:

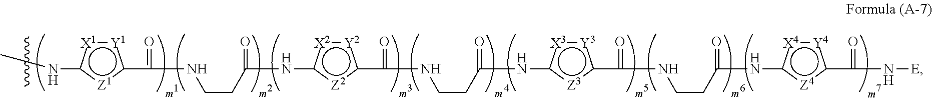

wherein:

E is an end subunit which comprises a moiety chosen from a heterocyclic group or a straight chain aliphatic group, which is chemically linked to its single neighbor;

X¹, Y¹, and Z¹ in each m¹ unit are independently selected from CR⁴, N, or NR⁵;

X², Y², and Z² in each m³ unit are independently selected from CR⁴, N, or NR⁵;

X³, Y³, and Z³ in each m⁵ unit are independently selected from CR⁴, N, or NR⁵;

X⁴, Y⁴, and Z⁴ in each m⁷ unit are independently selected from CR⁴, N, or NR⁵;

each R⁴ is independently H, —OH, halogen, C₁₋₆ alkyl, C₁₋₆ alkoxyl;

each R⁵ is independently H, C₁₋₆ alkyl or C₁₋₆alkylamine;

each m¹, m³, m⁵ and m⁷ are independently an integer between 0 and 5;

each m², m⁴ and m⁶ are independently an integer between 0 and 3; and

m¹+m²+m³+m⁴+m⁵+m⁶+m⁷ is between 3 and 15.

In some embodiments, m¹ is 3, and X¹, Y¹, and Z¹ in the first unit is respectively CH, N(CH₃), and CH; X¹, Y¹, and Z¹ in the second unit is respectively CH, N(CH₃), and N; and X¹, Y¹, and Z¹ in the third unit is respectively CH, N(CH₃), and N. In some embodiments, m³ is 1, and X², Y², and Z² in the first unit is respectively CH, N(CH₃), and CH. In some embodiments, m⁵ is 2, and X³, Y³, and Z³ in the first unit is respectively CH, N(CH₃), and N; X³, Y³, and Z³ in the second unit is respectively CH, N(CH₃), and N. In some embodiments, m⁷ is 2, and X⁴, Y⁴, and Z⁴ in the first unit is respectively CH, N(CH₃), and CH; X⁴, Y⁴, and Z⁴ in the second unit is respectively CH, N(CH₃), and CH. In some embodiments, each m², m⁴ and m⁶ are independently 0 or 1. In some embodiments, each of the X¹, Y¹, and Z¹ in each m¹ unit are independently selected from CH, N, or N(CH₃). In some embodiments, each of the X², Y², and Z² in each m³ unit are independently selected from CH, N, or N(CH₃). In some embodiments, each of the X³, Y³, and Z³ in each m⁵ unit are independently selected from CH, N, or N(CH₃). In some embodiments, each of the X⁴, Y⁴, and Z⁴ in each m⁷ unit are independently selected from CH, N, or N(CH₃). In some embodiments, each Z¹ in each m¹ unit is independently selected from CR⁴ or NR⁵. In some embodiments, each Z² in each m³ unit is independently selected from CR⁴ or NR⁵. In some embodiments, each Z³ in each m⁵ unit is independently selected from CR⁴ or NR⁵. In some embodiments, each Z⁴ in each m⁷ unit is independently selected from CR⁴ or NR⁵. In some embodiments, R⁴ is H, CH₃, or OH. In some embodiments, R⁵ is H or CH₃.

In some embodiments, for Formula (A-7), the sum of m², m⁴ and m⁶ is between 1 and 6. In some embodiments, for formula (A-7), the sum of m², m⁴ and m⁶ is between 2 and 6. In some embodiments, for Formula (A-7), the sum of m¹, m³, m⁵ and m⁷ is between 2 and 10. In some embodiments, the sum of m¹, m³, m⁵ and m⁷ is between 3 and 8. In some embodiments, for Formula (A-7), (m¹+m²+m³+m⁴+m⁵+m⁶+m⁷) is between 3 and 12. In some embodiments, (m¹+m²+m³+m⁴+m⁵+m⁶+m⁷) is between 4 and 10.

In some embodiments, for Formula (A-1) to (A-7), the first terminus comprises at least one beta-alanine moiety. In some embodiments, for Formula (A-1) to (A-7), the first terminus comprises at least two beta-alanine moieties. In some embodiments, for Formula (A-1) to (A-7), the first terminus comprises at least three or four β-alanine moieties.

The DNA recognition or binding moiety can include one or more subunits selected from the group consisting of:

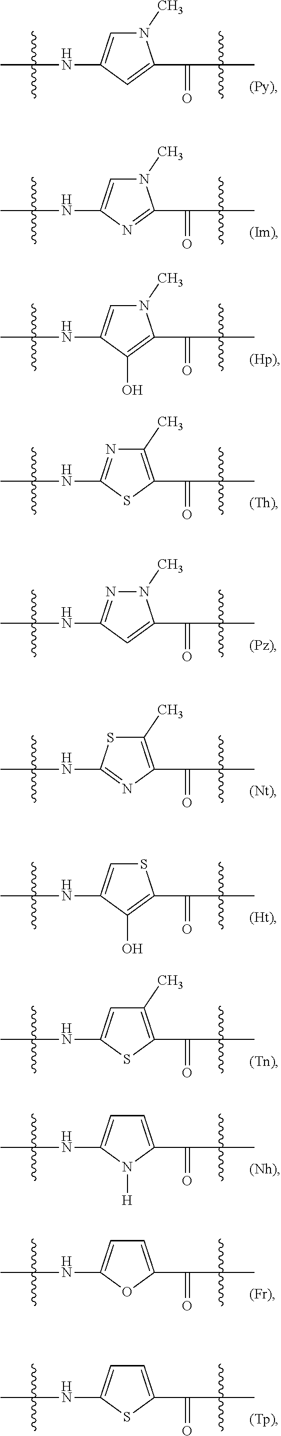

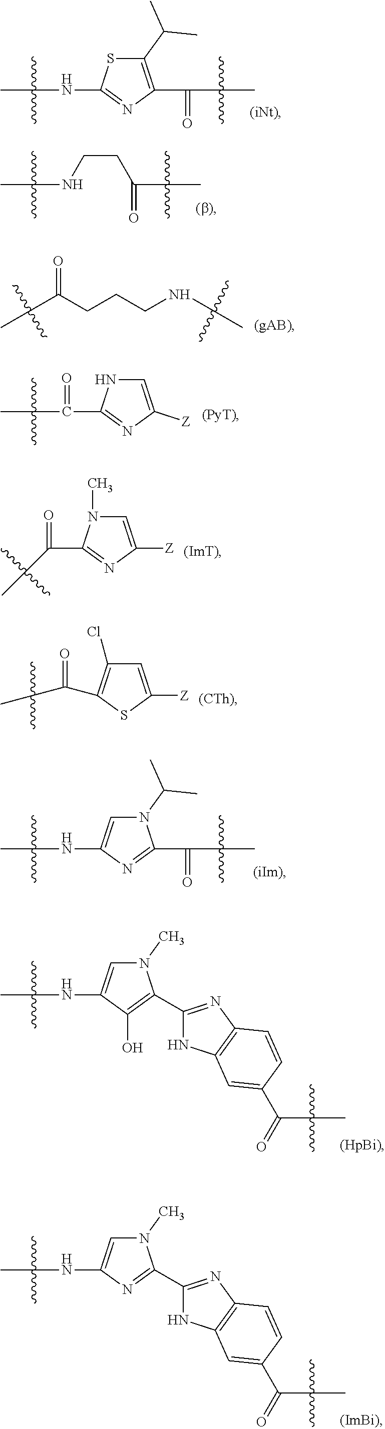

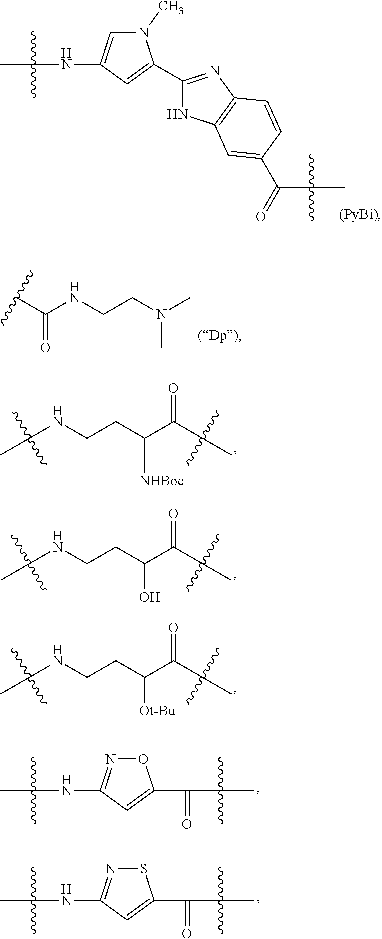

—NH-benzopyrazinylene-CO—, —NH-phenylene-CO—, —NH-pyridinylene-CO—, —NH-piperidinylene-CO—, —NH-pyrimidinylene-CO—, —NH-anthracenylene-CO—, —NH-quinolinylene-CO—, and

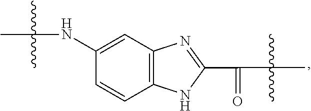

wherein Z is H, NH₂, C₁₋₆ alkyl, or C₁₋₆ alkylNH₂.

In some embodiments, Py is

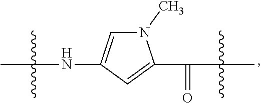 [SMILES: *Nc1cc(C(*)=O)n(C)c1]

### Im is

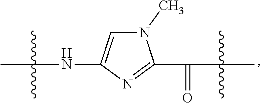 [SMILES: *Nc1cn(C)c(C(*)=O)n1]

### Hp is

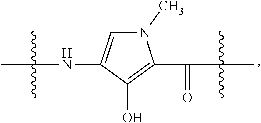 [SMILES: *Nc1cn(C)c(C(*)=O)c1O]

### Th is

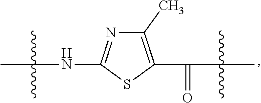 [SMILES: *Nc1nc(C)c(C(*)=O)s1]

### Pz is

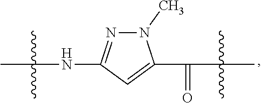 [SMILES: *Nc1cc(C(*)=O)n(C)n1]

### Nt is

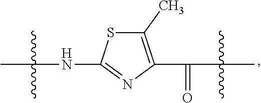 [SMILES: *Nc1nc(C(*)=O)c(C)s1]

### Tn is

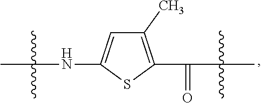 [SMILES: *Nc1cc(C)c(C(*)=O)s1]

### Nh is

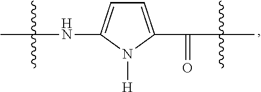 [SMILES: *Nc1ccc(C(*)=O)[nH]1]

iNt is

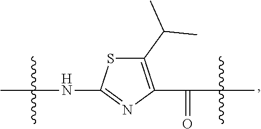 [SMILES: *Nc1nc(C(*)=O)c(C(C)C)s1]

iIm is

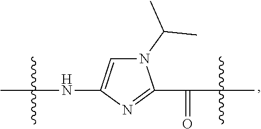 [SMILES: *Nc1cn(C(C)C)c(C(*)=O)n1]

### HpBi is

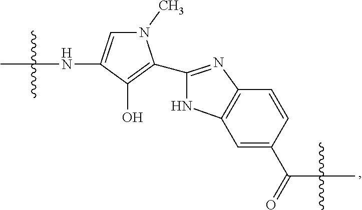 [SMILES: *Nc1cn(C)c(-c2nc3ccc(C(*)=O)cc3[nH]2)c1O]

### ImBi is

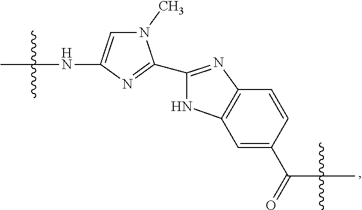 [SMILES: *Nc1cn(C)c(-c2nc3ccc(C(*)=O)cc3[nH]2)n1]

### PyBi is

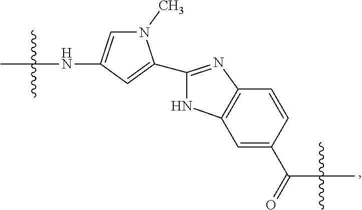 [SMILES: *Nc1cc(-c2nc3ccc(C(*)=O)cc3[nH]2)n(C)c1]

### Dp is

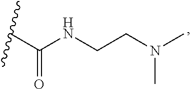 [SMILES: *C(=O)NCCN(C)C]

### —NH-benzopyrazinylene-CO— is

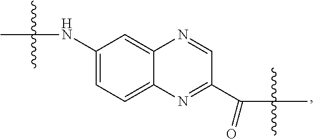 [SMILES: *Nc1ccc2nc(C(*)=O)cnc2c1]

### —NH-phenylene-CO— is

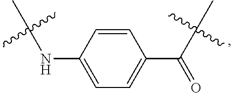 [SMILES: *Nc1ccc(C(*)=O)cc1]

### —NH-pyridinylene-CO— is

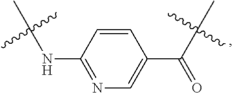 [SMILES: *Nc1ccc(C(*)=O)cn1]

### —NH-piperidinylene-CO— is

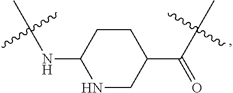 [SMILES: *NC1CCC(C(*)=O)CN1]

### —NH-pyrazinylene-CO— is

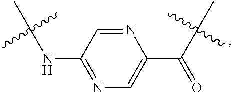 [SMILES: *Nc1cnc(C(*)=O)cn1]

### —NH-anthracenylene-CO— is

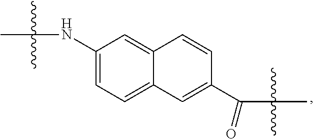 [SMILES: *Nc1ccc2cc(C(*)=O)ccc2c1]

### and —NH-quinolinylene-CO— is

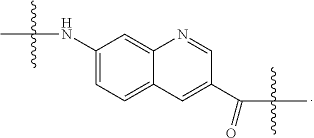 [SMILES: *Nc1ccc2cc(C(*)=O)cnc2c1]

In some embodiments, the first terminus comprises one or more subunits selected from the group consisting of optionally substituted N-methylpyrrole, optionally substituted N-methylimidazole, and β-alanine (β).

The first terminus in the molecules described herein has a high binding affinity to a sequence having multiple repeats of GAA and binds to the target nucleotide repeats preferentially over other nucleotide repeats or nucleotide sequences. In some embodiments, the first terminus has a higher binding affinity to a sequence having multiple repeats of GAA than to a sequence having repeats of CGG. In some embodiments, the first terminus has a higher binding affinity to a sequence having multiple repeats of GAA than to a sequence having repeats of CCG. In some embodiments, the first terminus has a higher binding affinity to a sequence having multiple repeats of GAA than to a sequence having repeats of CCTG. In some embodiments, the first terminus has a higher binding affinity to a sequence having multiple repeats of GAA than to a sequence having repeats of TGGAA. In some embodiments, the first terminus has a higher binding affinity to a sequence having multiple repeats of GAA than to a sequence having repeats of GGGGCC. In some embodiments, the first terminus has a higher binding affinity to a sequence having multiple repeats of GAA than to a sequence having repeats of CAG. In some embodiments, the first terminus has a higher binding affinity to a sequence having multiple repeats of GAA than to a sequence having repeats of CTG.

Due to the preferential binding between the first terminus and the target nucleotide repeat, the transcription modulation molecules described herein become localized around regions having multiple repeats of GAA. In some embodiments, the local concentration of the first terminus or the molecules described herein is higher near a sequence having multiple repeats of GAA than near a sequence having repeats of CGG. In some embodiments, the local concentration of the first terminus or the molecules described herein is higher near a sequence having multiple repeats of GAA than near a sequence having repeats of CCG. In some embodiments, the local concentration of the first terminus or the molecules described herein is higher near a sequence having multiple repeats of GAA than near a sequence having repeats of CCTG. In some embodiments, the local concentration of the first terminus or the molecules described herein is higher near a sequence having multiple repeats of GAA than near a sequence having repeats of TGGAA. In some embodiments, the local concentration of the first terminus or the molecules described herein is higher near a sequence having multiple repeats of GAA than near a sequence having repeats of GGGGCC. In some embodiments, the local concentration of the first terminus or the molecules described herein is higher near a sequence having multiple repeats of GAA than near a sequence having repeats of CTG. In some embodiments, the local concentration of the first terminus or the molecules described herein is higher near a sequence having multiple repeats of GAA than near a sequence having repeats of CAG.

The first terminus is localized to a sequence having multiple repeats of GAA and binds to the target nucleotide repeats preferentially over other nucleotide repeats. In some embodiments, the sequence has at least 2, 3, 4, 5, 8, 10, 12, 15, 20, 25, 30, 40, 50, 100, 200, 300, 400, or 500 repeats of GAA. In certain embodiments, the sequence comprises at least 1000 nucleotide repeats of GAA. In certain embodiments, the sequence comprises at least 500 nucleotide repeats of GAA. In certain embodiments, the sequence comprises at least 200 nucleotide repeats of GAA. In certain embodiments, the sequence comprises at least 100 nucleotide repeats of GAA. In certain embodiments, the sequence comprises at least 50 nucleotide repeats of GAA. In certain embodiments, the sequence comprises at least 20 nucleotide repeats of GAA.

In one aspect, the compounds of the present disclosure can bind to the repeated GAA of fxn than to GAA elsewhere in the subject's DNA

The polyamide composed of a pre-selected combination of subunits can selectively bind to the DNA in the minor groove. In their hairpin structure, antiparallel side-by-side pairings of two aromatic amino acids bind to DNA sequences, with a polyamide ring packed specifically against each DNA base. N-Methylpyrrole (Py) favors T, A, and C bases, excluding G; N-methylimidazole (Im) is a G-reader; and 3-hydroxyl-N-methylpyrrol (Hp) is specific for thymine base. The nucleotide base pairs can be recognized using different pairings of the amino acid subunits using the paring principle shown in Table 1A and 1B below. For example, an Im/Py pairing reads G⋅C by symmetry, a Py/Im pairing reads C⋅G, an Hp/Py pairing can distinguish T⋅A from A⋅T, G⋅C, and C⋅G, and a Py/Py pairing nonspecifically discriminates both A⋅T and T⋅A from G⋅C and C⋅G.

In some embodiments, the first terminus comprises Im corresponding to the nucleotide G; Py or beta corresponding to the nucleotide A; Py corresponding to the nucleotide A, wherein Im is N-alkyl imidazole, Py is N-alkyl pyrrole, and beta is β-alanine. In some embodiments, the first terminus comprises Im/Py to correspond to the nucleotide pair G/C, Py/beta or Py/Py to correspond to the nucleotide pair A/T, and wherein Im is N-alkyl imidazole (e.g., N-methyl imidazole), Py is N-alkyl pyrrole (e.g., N-methyl pyrrole), and beta is β-alanine.

TABLE 1A
Base paring for single amino acid subunit (Favored (+), disfavored (−).

| Subunit | G | C | A | T |
|---|---|---|---|---|
| Py | − | + | + | + |
| Im | + | − | − | |
| 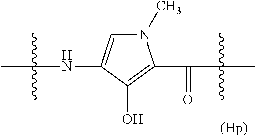 [SMILES: *Nc1cn(C)c(C(*)=O)c1O] | − | − | − | + |
| Hp | | | | |
| 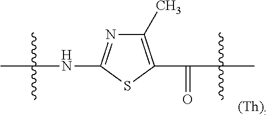 | − | − | + | + |
| 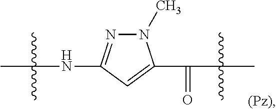 [SMILES: *Nc1cc(C(*)=O)n(C)n1] | − | − | + | + |
| 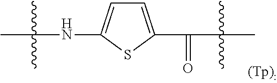 [SMILES: *Nc1ccc(C(*)=O)s1] | − | − | + | + |
| 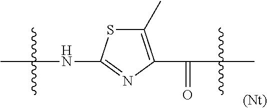 [SMILES: *Nc1nc(C(*)=O)c(C)s1] | + | − | − | − |
| 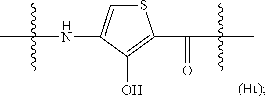 [SMILES: *Nc1csc(C(*)=O)c1O] | − | − | − | + |
| 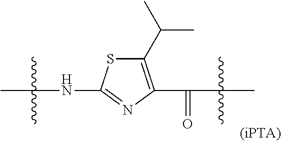 [SMILES: *Nc1nc(C(*)=O)c(C(C)C)s1] | + | − | − | − |
| 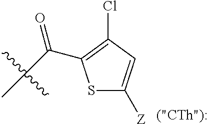 | − | − | − | + |
| 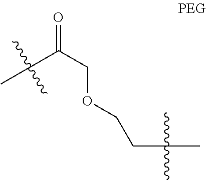 [SMILES: *CCOCC(*)=O] | − | + | + | + |
| 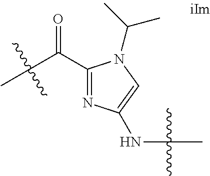 [SMILES: *Nc1cn(C(C)C)c(C(*)=O)n1] | + | − | − | − |
| 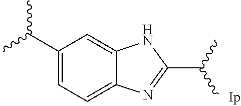 [SMILES: *c1ccc2nc(*)[nH]c2c1] | + | − | − | − |
| 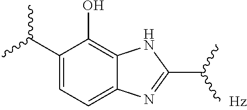 [SMILES: *c1nc2ccc(*)c(O)c2[nH]1] | − | − | − | + |
| 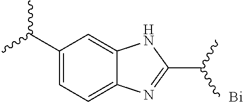 | − | − | − | + |
| 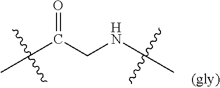 [SMILES: *NCC(*)=O] | − | − | − | − |
| 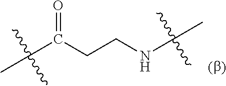 [SMILES: *NCCC(*)=O] | − | − | + | + |
| 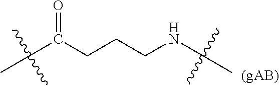 [SMILES: *NCCCC(*)=O] | − | − | + (as a part of the turn) | + (as a part of the turn) |
| 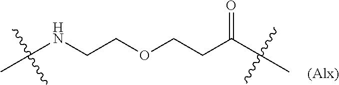 [SMILES: *NCCOCCC(*)=O] | − | + | − | − |
| 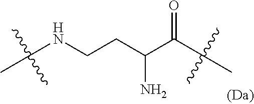 [SMILES: *NCCC(N)C(*)=O] | − | − | + | + |
| 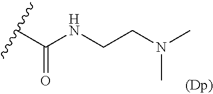 [SMILES: *C(=O)NCCN(C)C] | − | − | + | + |
| 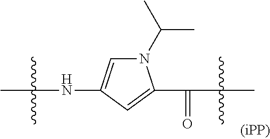 [SMILES: *Nc1cc(C(*)=O)n(C(C)C)c1] | − | − | + | + |
| 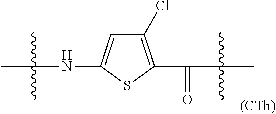 | + | + | − | − |
| 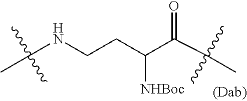 [SMILES: *NCCC(NC(=O)OC(C)(C)C)C(*)=O] | − | − | + | + |
| 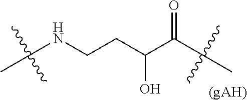 [SMILES: *NCCC(O)C(*)=O] | − | − | + | + |
| 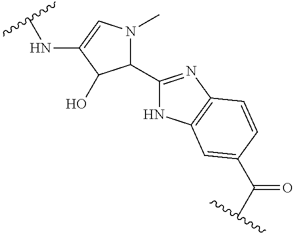 [SMILES: *NC1=CN(C)C(c2nc3ccc(C(*)=O)cc3[nH]2)C1O] | WW* (bind to two nucleotides with same selectivity as Hp-Py) | | | |
| HpBi | | | | |
| 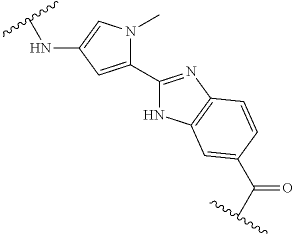 [SMILES: *Nc1cc(-c2nc3ccc(C(*)=O)cc3[nH]2)n(C)c1] | WW* (bind to two nucleotides with same selectivity as PyPy) | | | |
| PyBi | | | | |
| 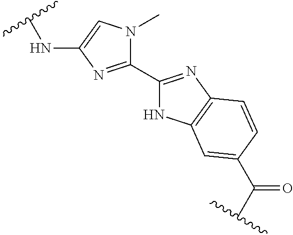 [SMILES: *Nc1cn(C)c(-c2nc3ccc(C(*)=O)cc3[nH]2)n1] | GW* (bind to two nucleotides with same selectivity as Im-Py) | | | |
| ImBi | | | | |

*The subunit HpBi, ImBi, and PyBi function as a conjugate of two monomer subunits and bind to two nucleotides. The binding property of HpBi, ImBi, and PyBi corresponds to Hp-Py, Im-Py, and Py-Py respectively.

TABLE 1B
Base pairing for hairpin polyamide.

| | G•C | C•G | T•A | A•T |
|---|---|---|---|---|
| Im/β | + | − | − | − |
| β/Im | − | + | − | − |
| Py/β | − | − | + | + |
| β/Py | − | − | + | + |
| β/β | − | − | + | + |
| Py/Py | − | − | + | + |
| Im/Im | − | − | − | − |
| Im/Py | + | − | − | − |
| Py/Im | − | + | − | − |
| Th/Py | − | − | + | − |
| Py/Th | − | − | − | + |
| Th/Im | + | − | − | − |
| Im/Th | − | + | − | − |
| β/Th | − | − | + | − |
| Th/β | − | − | − | + |
| Hp/Py, | − | − | + | − |
| Py/Hp, | − | − | − | + |
| Hp/Im | + | − | − | − |
| Im/Hp | − | + | − | − |
| Tn/Py | − | − | + | + |
| Py/Tn, | − | − | + | + |
| Ht/Py, | − | − | + | + |
| Py/Ht, | − | − | + | + |
| Bi/Py, | − | − | + | + |
| Py/Bi, | − | − | + | + |
| β/Bi | − | − | + | + |
| Bi/β | − | − | + | + |
| Bi/Im, | − | + | − | − |
| Im/Bi, | + | − | − | − |
| Tp/Py, | − | − | + | + |
| Py/Tp, | − | − | + | + |
| β/Tρ | − | − | + | + |
| Tρ/β | − | − | + | + |
| Tp/Im, | − | + | − | − |
| Im/Tp | + | − | − | − |
| Tp/Tp | − | − | + | + |
| Tp/Tn | − | − | + | + |
| Tn/Tp | − | − | + | + |
| Hz/Py, | − | − | + | − |
| Py/Hz, | − | − | − | + |
| Ip/Py | + | − | − | − |
| Py/Ip, | − | + | − | − |
| Bi/Hz, | − | − | + | + |
| Hz/Bi, | − | − | + | + |
| Bi/Bi | − | + | + | + |
| Th/Py, | − | − | + | + |
| Py/Th | − | − | + | + |
| Im/gAB | + | − | − | − |
| gAB/Im | − | + | − | − |
| Py/gAB | + | − | − | − |
| gAB/Py | − | + | − | − |
| gAB/β | − | − | + | + |
| β/gAB | − | − | + | + |
| Im/Dp | + | − | − | − |
| Dp/Im | − | + | − | − |
| Py/Dp | − | − | + | + |
| Dp/Py | − | − | + | + |
| Dp/β | − | − | + | + |

Each of HpBi, ImBi, and PyBi can bind to two nucleotides and have binding properties corresponding to Hp-Py, Im-Py, and Py-Py respectively. HpBi, ImBi, and PyBi can be paired with two monomer subunits or with themselves in a hairpin structure to bind to two nucleotide pairs.

The monomer subunits of the polyamide can be strung together based on the paring principles shown in Table 1A and Table 1B. The monomer subunits of the polyamide can be strung together based on the paring principles shown in Table 1C and Table 1D.

Table 1C shows an example of the monomer subunits that can bind to the specific nucleotide. The first terminus can include a polyamide described having several monomer subunits stung together, with a monomer subunit selected from each row. For example, the polyamide can include Im-β-Py that binds to GAA, with Im selected from the first G column, p from the A column, and Py from the second A column. The polyamide can be any combinations that bind to the subunits of GAA, with a subunit selected from each column in Table 1C, wherein the subunits are strung together following the GAA order.

In addition, the polyamide can also include a partial or multiple sets of the five subunits, such as 1.5, 2, 2.5, 3, 3.5, or 4 sets of the three subunits. The polyamide can include 3, 4, 5, 6, 7, 8, 9, 10, 12, 14, and 16 monomer subunits. The multiple sets can be joined together by W. In addition to the five subunits or ten subunits, the polyamide can also include 1-4 additional subunits that can link multiple sets of the five subunits.

The polyamide can include monomer subunits that bind to 2, 3, 4, or 5 nucleotides of GAA. For example, the polyamide can bind to GA, AA, GAA, AAG, AGA, GAAG, AAGA, GAAGA or GAAGAA. The polyamide can include monomer subunits that bind to 3, 4, 5, 6, 7, 8, 9, or 10 nucleotides of GAA repeats. The nucleotides can be joined by W

The monomer subunit, when positioned as a terminal unit, does not have an amine, carbonyl, or a carboxylic acid group at the terminal. The amine or carboxylic acid group in the terminal is replaced by a hydrogen. For example, Py, when used as a terminal unit, is understood to have the structure of

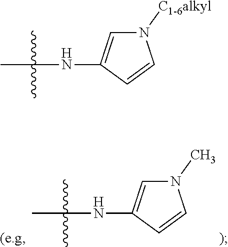

and Im, when positioned as a terminal unit, is understood to have the structure of

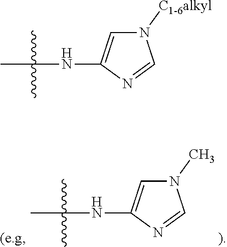

In addition, when Py or Im is used as a terminal unit, Py and Im can be respectively replaced by

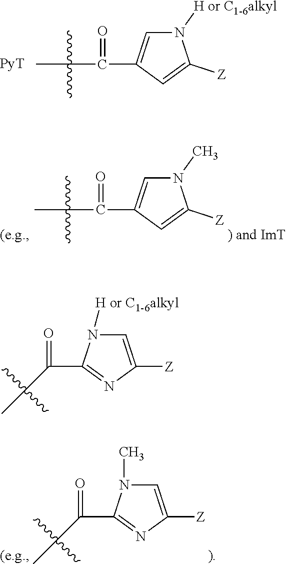

The linear polyamide can have nonlimiting examples including but not limited β-Py-Im, Im-Py-β-Im-Py-β-Im-Py, Im-Py-β-Im-Py-Py-Im-β, Im-Py-Py-Im-Py-β-Im-β, and any combinations thereof.

TABLE 1C
Examples of monomer subunits in a
linear polyamide that binds to GAA.

| Nucleotide | G | A | A |
|---|---|---|---|
| Subunit that selectively | Im or ImT | Py | Py |
| binds to nucleotide | iIm or iImT | Th | Th |
| | PEG | Pz | Pz |
| | CTh | Tp | Tp |
| | Nt | PEG | PEG |
| | iPTA | β | β |
| | Ip | iPP | iPP |
| | CTh | Da | Da |
| | | Dp | Dp |
| | | Dab | Dab |
| | | gAH | gAH |

The DNA-binding moiety can also include a hairpin polyamide having subunits that are strung together based on the pairing principle shown in Table 1B. Table 1D shows some examples of the monomer subunit pairs that selectively bind to the nucleotide pair. The hairpin polyamide can include 2n monomer subunits (n is an integer in the range of 2-8), and the polyamide also includes a W in the center of the 2n monomer subunits. W can be —(CH₂)ₐ—NR¹—(CH₂)_{b}—, —(CH₂)ₐ—, —(CH₂)ₐ—O—(CH₂)_{b}—, —(CH₂)ₐ—CH(NHR¹)—, —(CH₂)a-CH(NHR¹)—, —(CR²R³)ₐ— or —(CH₂)ₐ—CH(NR¹₃)⁺—(CH₂)_{b}—, wherein each a is independently an integer between 2 and 4; R¹ is H, an optionally substituted C₁₋₆ alkyl, an optionally substituted C₃₋₁₀ cycloalkyl, an optionally substituted C₆₋₁₀ aryl, an optionally substituted 4-10 membered heterocyclyl, or an optionally substituted 5-10 membered heteroaryl; each R² and R³ are independently H, halogen, OH, NHAc, or C₁₋₄ alky. In some embodiments, W is —(CH₂)—CH(NH₃)⁺—(CH₂)— or —(CH₂)—CH₂CH(NH₃)⁺—. In some embodiments, R¹ is H. In some embodiments, R¹ is C₁₋₆ alkyl optionally substituted by 1-3 substituents selected from —C(O)-phenyl. In some embodiments, W is —(CR²R³)—(CH₂)a- or —(CH₂)ₐ—(CR²R³)—(CH₂)_{b}—, wherein each a is independently 1-3, b is 0-3, and each R² and R³ are independently H, halogen, OH, NHAc, or C₁₋₄ alky. W can be an aliphatic amino acid residue shown in Table 4 such as gAB.

Because the target gene can include multiple repeats of GAA, the subunits can be strung together to bind at least two, three, four, five, six, seven, eight, nine, or ten nucleotides in one or more GAA repeat (e.g., GAAGAAGAAGAA). For example, the polyamide can bind to the GAA repeat by binding to a partial copy, a full copy, or a multiple repeats of GAA such as GA, AA, GAA, AAG, AGA, GAAG, AAGA, GAAGA or GAAGAA. For example, the polyamide can include Im-Py-β-W-Py-β-Py that binds to GAA and its complementary nucleotides on a double strand DNA, in which the Im/Py pair binds to the G⋅C, the Py/β pair binds to A⋅T, and the β/Py pair binds to G⋅A. In another example Im-Py-β-Im-W-β-Py-β-Py that binds to GAAG and its complementary nucleotides on a double strand DNA, in which the Im/Py pair binds to the G⋅C, the Py/P pair binds to A⋅T, the β/Py pair binds to G⋅A, and the Im/β pair binds to the G⋅C, W can be an aliphatic amino acid residue such as gAB or other appropriate spacers as shown in Table 4. In another example, Im-Py-β-Im-gAB-Im-Py binds to with a part of the complementary nucleotides (ACG) on the double strand DNA, in which Im binds to G, Py binds to A, □/Py binds to the A⋅T, Im/Im binds to G⋅C.

Some additional examples of the polyamide include but are not limited to Im-Py-Py-Im-gAB-Py-Im-Im-Py; Im-Py-Py-Im-gAB-Py-Im-Im-PyT; Im-Py-Py-Im-gAB-Py-Im-Im-β; Im-Py-Py-Im-gAB-Py-Im-Im-β-G; Im-β-β-Py-Im-gAB-Py-Im-Im-β; Im-β-Py-Im-gAB-Py-Im-Im-β-G; Im-β-Py-Im-gAB-Py-Im-Im-Py; Im-β-Py-Im-gAB-Py-Im-Im-PyT; Py-Py-Im-β-gAB-Im-Py-Im-Im; Py-Py-Im-β-gAB-Im-Py-Im-ImT; Py-Py-Im-Py-gAB-Im-Py-Im-Im; Py-Py-Im-Py-gAB-Im-Py-Im-ImT; Py-Py-Im-β-gAB-Im-β-Im-Im; Py-Py-Im-β-gAB-Im-β-Im-ImT; Py-Py-Im-Py-gAB-Im-β-Im-Im; Py-Py-Im-Py-gAB-Im-β-Im-ImT; Im-β-Py-gAB-Im-Im-Py; Im-β-Py-gAB-Im-Im-PyT; Im-β-Py-gAB-Im-Im-β; Im-β-Py-gAB-Im-Im-β-G; Im-Py-Py-gAB-Im-Im-β; Im-Py-Py-gAB-Im-Im-β-G; Im-Py-Py-gAB-Im-Im-Py; Im-Py-Py-gAB-Im-Im-PyT; Im-p-Py-gAB-Im-Im-Py; and Im-β-Py-gAB-Im-Im-PyT; wherein G may be hydrogen, alkyl, alkenyl, alkynyl, or —C(O)—R_{B}; and R_{B} may be a hydrogen, C₁-C₆ alkyl, C₁-C₆ alkenyl, or C₁-C₆ alkynyl group. In some embodiments, the hairpin polyamide has a structure of Im-Py-β-Im-gAB-Im-Py; Im-Py-β-Im-gAB-Im-Py-β-Im; Py-β-Im-gAB-Im-Py-β-Im; or β-Im-gAB-Im-Py-β-Im.

TABLE 1D
Examples of monomer pairs in a hairpin or
H-pin polyamide that binds to CAG or CTG.

| Nucleotide | G•C | A•T | A•T |
|---|---|---|---|
| Subunit pairs that selectively | Im/β | Py/β | Py/β |
| binds to nucleotide | Im/Py | β/Py | β/Py |
| | Th/Im | β/β | β/β |
| | Hp/Im | Py/Py | Py/Py |
| | Im/Bi | Py/Th | Py/Th |
| | Im/Tp | Th/β | Th/β |
| | Ip/Py | Py/Hp, | Py/Hp, |
| | Im/gAB | Tn/Py | Tn/Py |
| | Py/gAB | Py/Tn, | Py/Tn, |
| | Im/Dp | Ht/Py, | Ht/Py, |
| | | Py/Ht, | Py/Ht, |
| | | Bi/Py, | Bi/Py, |
| | | Py/Bi, | Py/Bi, |
| | | β/Bi | β/Bi |
| | | Bi/β | Bi/β |
| | | Tp/Py, | Tp/Py, |
| | | Py/Tp, | Py/Tp, |
| | | β/Tρ | β/Tρ |
| | | Tρ/β | Tρ/β |
| | | Tp/Tp | Tp/Tp |
| | | Tp/Tn | Tp/Tn |
| | | Tn/Tρ | Tn/Tp |
| | | Py/Hz, | Py/Hz, |
| | | Bi/Hz, | Bi/Hz, |
| | | Hz/Bi, | Hz/Bi, |
| | | Bi/Bi | Bi/Bi |
| | | Th/Py, | Th/Py, |
| | | Py/Th | Py/Th |
| | | gAB/β | gAB/β |
| | | β/gAB | β/gAB |
| | | Py/Dp | Py/Dp |
| | | Dp/Py | Dp/Py |
| | | Dp/β | Dp/β |

Recognition of a nucleotide repeat or DNA sequence by two antiparallel polyamide strands depends on a code of side-by-side aromatic amino acid pairs in the minor groove, usually oriented N to C with respect to the 5′ to 3′ direction of the DNA helix. Enhanced affinity and specificity of polyamide nucleotide binding is accomplished by covalently linking the antiparallel strands. The “hairpin motif” connects the N and C termini of the two strands with a W (e.g., gamma-aminobutyric acid unit (gamma-turn)) to form a folded linear chain. The “H-pin motif” connects the antiparallel strands across a central or near central ring/ring pairs by a short, flexible bridge.

The DNA-binding moiety can also include a H-pin polyamide having subunits that are strung together based on the pairing principles shown in Table 1A and/or Table 1B. Table 1C shows some examples of the monomer subunit that selectively binds to the nucleotide, and Table 1D shows some examples of the monomer subunit pairs that selectively bind to the nucleotide pair. The h-pin polyamide can include 2 strands and each strand can have a number of monomer subunits (each strand can include 2-8 monomer subunits), and the polyamide also includes a bridge L₁ to connect the two strands in the center or near the center of each strand. At least one or two of the monomer subunits on each strand are paired with the corresponding monomer subunits on the other stand following the paring principle in Table 1D to favor binding of either G⋅C or C⋅G, A⋅T, or T⋅A pair, and these monomer subunit pairs are often positioned in the center, close to center region, at or close to the bridge that connects the two strands. In some instances, the H-pin polyamide can have all of the monomer subunits be paired with the corresponding monomer subunits on the antiparallel strand based on the paring principle in Table 1B and 1D to bind to the nucleotide pairs on the double strand DNA. In some instances, the H-pin polyamide can have a part of the monomer subunits (2, 3, 4, 5, or 6) be paired with the corresponding monomer subunits on the antiparallel strand based on the binding principle in Table 1B and 1D to bind to the nucleotide pairs on the double strand DNA, while the rest of the monomer subunit binds to the nucleotide based on the binding principle in Table 1A and 1C but does not pair with the monomer subunit on the antiparallel strand. The h-pin polyamide can have one or more overhanging monomer subunit that binds to the nucleotide but does not pair with the monomer subunit on the antiparallel strand.

Another polyamide structure that derives from the h-pin structure is to connect the two antiparallel strands at the end through a bridge, while only the two monomer subunits that are connected by the bridge form a pair that bind to the nucleotide pair G⋅C or C⋅G based on the binding principle in Table 1B/1D, but the rest of the monomer subunits on the strand form an overhang, bind to the nucleotide based on the binding principle in Table 1A and/or 1C and do not pair with the monomer subunit on the other strand.

The bridge can be is a bivalent or trivalent group selected from

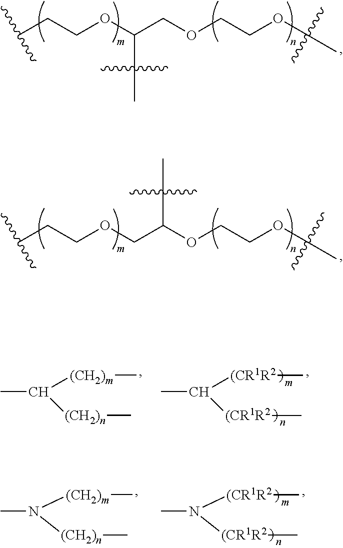

a C₁₋₁₀ alkylene, —NH—C₀₋₆ alkylene-C(O)—, —N(CH₃)—C₀₋₆ alkylene, and

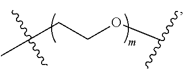

—(CH₂)ₐ—NR¹—(CH₂)_{b}—, —(CH₂)ₐ—, —(CH₂)ₐ—O—(CH₂)_{b}—, —(CH₂)ₐ—CH(NHR¹)—, —(CH₂)ₐ—CH(NHR¹)—, —(CR²R³)ₐ or —(CH₂)ₐ—CH(NR¹₃)⁺—(CH₂)_{b}—, wherein m is an integer in the range of 0 to 10; n is an integer in the range of 0 to 10; each a is independently an integer between 2 and 4; R¹ is H, an optionally substituted C₁₋₆ alkyl, an optionally substituted C₃₋₁₀ cycloalkyl, an optionally substituted C₆₋₁₀ aryl, an optionally substituted 4-10 membered heterocyclyl, or an optionally substituted 5-10 membered heteroaryl; each R² and R³ are independently H, halogen, OH, NHAc, or C₁₋₄ alky. In some embodiments, W is —(CH₂)—CH(NH₃)⁺—(CH₂)— or —(CH₂)—CH₂CH(NH₃)⁺—. In some embodiments, R¹ is H. In some embodiments, R¹ is C₁₋₆ alkyl optionally substituted by 1-3 substituents selected from —C(O)-phenyl. In some embodiments, L₁ is —(CR²R³)—(CH₂)ₐ or —(CH₂)ₐ—(CR²R³)—(CH₂)_{b}—, wherein each a is independently 1-3, b is 0-3, and each R² and R³ are independently H, halogen, OH, NHAc, or C₁-4 alky. L₁ can be a C₂₋₉ alkylene or (PEG)₂₋₈.

Some additional examples of the polyamide include but are not limited to Im-Py-Py-Im (Linked in the middle—either position 2 or 3) to Py-Py-Py-Py, Im-Py-Py-Im (Linked in the middle—position 3 py and Py) to Im-Py-β-Py-Py, Im-Py-β-Im (linked to the bolded position) Im-Py; Im-Py-β-Im (linked in the middle, either position 2 or 3) Im-Py-b-Im; Py-β-Im (linked to the middle position bolded) Im-Py-β-Im; or β-Im (linked at bolded position) Im-Py-β-Im.

### Second Terminus—Regulatory Binding Moiety

In certain embodiments, the regulatory molecule is chosen from a nucleosome remodeling factor (NURF), a bromodomain PHD finger transcription factor (BPTF), a ten-eleven translocation enzyme (TET), methylcytosine dioxygenase (TET1), a DNA demethylase, a helicase, an acetyltransferase, and a histone deacetylase (“HDAC”).

The binding affinity between the regulatory protein and the second terminus can be adjusted based on the composition of the molecule or type of protein. In some embodiments, the second terminus binds the regulatory molecule with an affinity of less than about 600 nM, about 500 nM, about 400 nM, about 300 nM, about 250 nM, about 200 nM, about 150 nM, about 100 nM, or about 50 nM. In some embodiments, the second terminus binds the regulatory molecule with an affinity of less than about 300 nM. In some embodiments, the second terminus binds the regulatory molecule with an affinity of less than about 200 nM. In some embodiments, the polyamide is capable of binding the DNA with an affinity of greater than about 200 nM, about 150 nM, about 100 nM, about 50 nM, about 10 nM, or about 1 nM. In some embodiments, the polyamide is capable of binding the DNA with an affinity in the range of about 1-600 nM, 10-500 nM, 20-500 nM, 50-400 nM, 100-300 nM, or 50-200 nM.

In some embodiments, the second terminus comprises one or more optionally substituted C₆₋₁₀ aryl, optionally substituted C₄₋₁₀ carbocyclic, optionally substituted 4 to 10 membered heterocyclic, or optionally substituted 5 to 10 membered heteroaryl.

In some embodiments, the protein-binding moiety binds to the regulatory molecule that is selected from the group consisting of a CREB binding protein (CBP), a P300, an O-linked β-N-acetylglucosamine-transferase- (OGT-), a P300-CBP-associated-factor- (PCAF-), histone methyltransferase, histone demethylase, chromodomain, a cyclin-dependent-kinase-9- (CDK9-), a nucleosome-remodeling-factor-(NURF-), a bromodomain-PHD-finger-transcription-factor- (BPTF-), a ten-eleven-translocation-enzyme- (TET-), a methylcytosine-dioxygenase- (TET1-), histone acetyltransferase (HAT), a histone deacetylase (HDAC), a host-cell-factor-1(HCF1-), an octamer-binding-transcription-factor- (OCT1-), a P-TEFb-, a cyclin-T1-, a PRC2-, a DNA-demethylase, a helicase, an acetyltransferase, a histone-deacetylase, methylated histone lysine protein.

In some embodiments, the second terminus comprises a moiety that binds to an O-linked β-N-acetylglucosamine-transferase (OGT), or CREB binding protein (CBP). In some embodiments, the protein binding moiety is a residue of a compound that binds to an O-linked β-N-acetylglucosamine-transferase(OGT), or CREB binding protein (CBP).

In some embodiments, the second terminus comprises JQ1, iBET762, OTX015, RVX208, or AU1. In some embodiments, the second terminus comprises JQ1. In some embodiments, the second terminus comprises a moiety that binds to a bromodomain protein.

In some embodiments, the second terminus comprises one or more optionally substituted C₆₋₁₀ aryl, optionally substituted C₄₋₁₀ carbocyclic, optionally substituted 4 to 10 membered heterocyclic, or optionally substituted 5 to 10 membered heteroaryl. In some embodiments, the second terminus comprises a diazine or diazepine ring, wherein the diazine or diazepine ring is fused with a C₆₋₁₀ aryl or a 5-10 membered heteroaryl ring comprising one or more heteroatom selected from S, N and O. In some embodiments, the second terminus comprises at least one 5-10 membered heteroaryl group having at least two nitrogen atoms.

In some embodiments, the second terminus comprises an optionally substituted bicyclic or tricyclic structure. In some embodiments, the optionally substituted bicyclic or tricyclic structure comprises a diazepine ring fused with a thiophene ring.

In some embodiments, the second terminus comprises an optionally substituted bicyclic structure, wherein the bicyclic structure comprises a diazepine ring fused with a thiophene ring. In some embodiments, the second terminus comprises at least one group selected from an optionally substituted diazine, an optionally substituted diazepine, and an optionally substituted phenyl.

In some embodiments, the second terminus comprises an optionally substituted tricyclic structure, wherein the tricyclic structure is a diazepine ring that is fused with a thiophene and a triazole.

In some embodiments, the second terminus comprises a moiety capable of binding to the regulatory protein, and the moiety is from a compound capable of binding to the regulatory protein.

In some embodiments, the second terminus comprises JQ1, JQ-1, OTX015, RVX208 acid, or RVX208 hydroxyl.

In some embodiments, the second terminus comprises a moiety that binds to a bromodomain protein. In certain embodiments, the regulatory molecule is a bromodomain-containing protein chosen from BRD2, BRD3, BRD4, and BRDT.

In certain embodiments, the regulatory molecule is BRD4. In certain embodiments, the recruiting moiety is a BRD4 activator. In certain embodiments, the BRD4 activator is chosen from JQ-1, OTX015, RVX208 acid, and RVX208 hydroxyl.

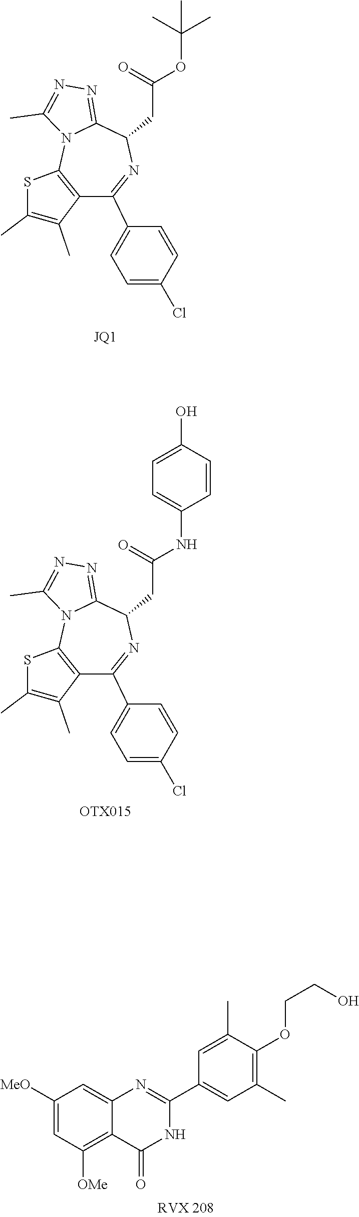

In certain embodiments, the regulatory molecule modulates the rearrangement of histones.

In certain embodiments, the regulatory molecule modulates the glycosylation, phosphorylation, alkylation, or acylation of histones.

In certain embodiments, the regulatory molecule is a transcription factor.

In certain embodiments, the regulatory molecule is an RNA polymerase.

In certain embodiments, the regulatory molecule is a moiety that regulates the activity of RNA polymerase.

In certain embodiments, the regulatory molecule interacts with TATA binding protein.

In certain embodiments, the regulatory molecule interacts with transcription factor II D.

In certain embodiments, X binds to the regulatory molecule but does not inhibit the activity of the regulatory molecule. In certain embodiments, X binds to the regulatory molecule and inhibits the activity of the regulatory molecule. In certain embodiments, X binds to the regulatory molecule and increases the activity of the regulatory molecule.

In certain embodiments, X binds to the active site of the regulatory molecule. In certain embodiments, X binds to a regulatory site of the regulatory molecule.

In some embodiments, the second terminus is a compound of Formula 7, or a pharmaceutically acceptable salt thereof:

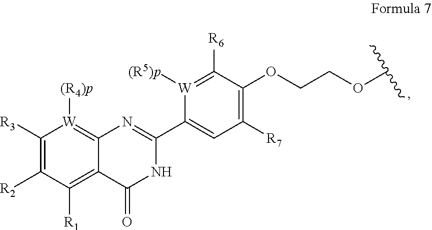

wherein; R₁ and R₃ are each independently selected from alkoxy, alkyl, amino, halogen, and hydrogen; R₂ is selected from alkoxy, alkyl, alkenyl, alkynyl, amide, amino, halogen, and hydrogen; R₆ and R₇ are each independently selected from alkyl, alkoxy, amino, halogen, and hydrogen; R₅ is hydrogen; each W is independently selected from C and N, wherein if W is N, then p is 0 or 1, and if W is C, then p is 1; for W—(R₄)ₚ, if W is C, p is 1 and R₄ is H, or if W is N, then p is 0; if R₁ is hydrogen, then R₃ is alkoxy; if R₃ is hydrogen, then R₁ is selected from amino and alkoxy; and at least one of R₆ and R₇ is independently selected from alkyl, alkoxy, amino, and halogen, and

a bromodomain binding moiety having the structure of Formula 9, or a pharmaceutically acceptable salt thereof:

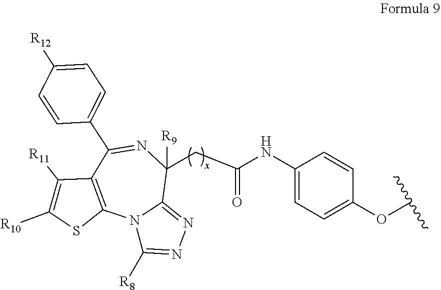

wherein; R₈, R₁₀, and R₁₁ are each independently selected from hydrogen, methyl, ethyl, and halomethyl; R₉ is selected from hydrogen, C₁-C₆ alkyl group, and substituted C₁-C₆ alkyl group; R₁₂ is selected from halogen, aryl, substituted aryl, amino, and amido; and X is an integer from 1 to 6.

In some embodiments, the second terminus comprises a compound having the structure of Formula 7:

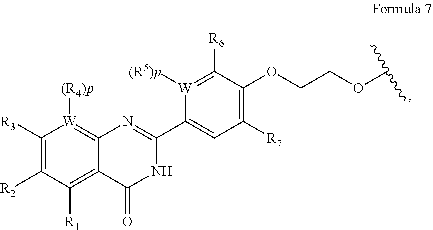

wherein;

R₁ and R₃ are each independently selected from alkoxy, alkyl, amino, halogen, and hydrogen;

R₂ is selected from alkoxy, alkyl, alkenyl, alkynyl, amide, amino, halogen, and hydrogen;
R₆ and R₇ are each independently selected from alkyl, alkoxy, amino, halogen, and hydrogen;
R₅ is hydrogen;

each W is independently selected from C and N, wherein if W is N, then p is 0 or 1, and if W is C, then p is 1; for W—(R₄)ₚ, if W is C, p is 1 and R₄ is H, or if W is N, then p is 0; if R₁ is hydrogen, then R₃ is alkoxy; if R₃ is hydrogen, then R₁ is selected from amino and alkoxy; and at least one of R₆ and R₇ is independently selected from alkyl, alkoxy, amino, and halogen.

In some embodiments, the second terminus is a compound of Formula 7, a bromodomain binding moiety having the structure of formula 9 and a bromodomain binding moiety having the structure of Formula 20:

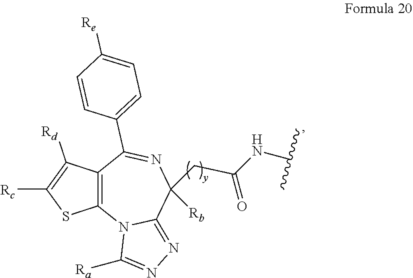

wherein; R_{b} is a hydrogen, C₁-C₆ alkyl group, or substituted C₁-C₆ alkyl group; Rₐ, R_{c}, and R_{d} are each independently hydrogen, methyl, ethyl, or halomethyl; Rₑ is a halogen, an aryl, a substituted aryl, amino, or amido group; and y is an integer from 1 to 6.

In some embodiments, the bromodomain binding moiety has a structure of Formula 8, or a pharmaceutically acceptable salt thereof:

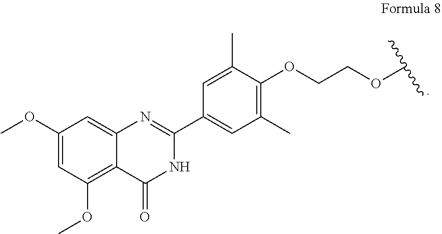 [SMILES: *OCCOc1c(C)cc(-c2nc3cc(OC)cc(OC)c3c(=O)[nH]2)cc1C]

In some embodiments, the second terminus comprises a compound having the structure of Formula (9-A), or a pharmaceutically acceptable salt thereof:

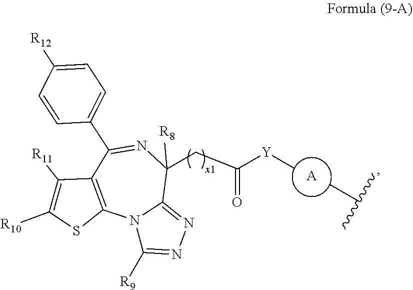

wherein;

Ring A is absent or a 6-membered monocyclic aryl or heteroaryl;

Y is —NH— or —O—;

R⁸ is hydrogen or C₁₋₆ alkyl;

R⁹, R¹⁰, and R¹¹ are each independently selected from hydrogen, optionally substituted C₁₋₆ alkyl, C₁₋₆ haloalkyl, or C₁₋₆ hydroxyalkyl;

R¹² is selected from hydrogen, halogen, —NO₂, —CN, optionally substituted aryl, optionally substituted C₁₋₆ alkyl, C₁₋₆ haloalkyl, or C₁₋₆ hydroxyalkyl;

or R¹² is —NR^{A}R^{B}, wherein
R^{A} and R^{B} are each independently hydrogen, optionally substituted C₁₋₆ alkyl or C₁₋₆ heteroalkyl; and

x1 is an integer from 1-6.

In some embodiments, Ring A is 6-membered monocyclic aryl or heteroaryl. In some embodiments Ring A is phenyl. In some embodiments, Ring A is 6-membered monocyclic heteroaryl. In some embodiments, Ring A is pyridine or pyrimidine.

In some embodiments, Ring A is absent.

In some embodiments, Y is —NH—. In some embodiments, Y is —O—.

In some embodiments, R⁸ is hydrogen.

In some embodiments, R⁹, R¹⁰, and R¹¹ are each independently selected from optionally substituted C₁₋₆ alkyl, C₁₋₆ haloalkyl, or C₁₋₆ hydroxyalkyl. In some embodiments, R⁹, R¹⁰, and R¹¹ are each independently selected from optionally substituted C₁₋₆ alkyl. In some embodiments, In some embodiments, R⁹, R¹⁰, and R¹¹ are each independently methyl, ethyl, or propyl. In some embodiments, In some embodiments, R⁹, R¹⁰, and R¹¹ are each independently methyl.

In some embodiments, R¹² is selected from hydrogen, halogen, optionally substituted C₁₋₆ alkyl, C₁₋₆ haloalkyl, or C₁₋₆ hydroxyalkyl. In some embodiments, R¹² is bromo, chloro, or fluoro.

In some embodiments, R¹² is —NR^{A}R^{B}, wherein R^{A} and R^{B} are each independently hydrogen, optionally substituted C₁₋₆ alkyl.

In some embodiments, x1 is an integer from 1-5, 1-4, 1-3, or 1-2. In some embodiments, x1 is 1. In some embodiments, x1 is 2.

In some embodiments, the second terminus comprises a compound having the structure of Formula (9-B), or a pharmaceutically acceptable salt thereof:

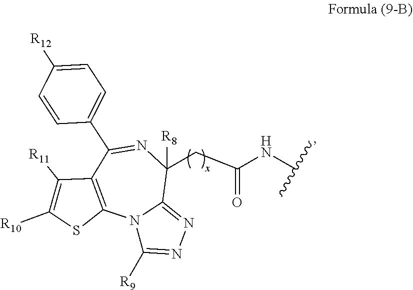

wherein;

R₈, R₁₀, and R₁₁ are each independently selected from hydrogen, methyl, ethyl, and halomethyl;

R₉ is selected from hydrogen, C₁-C₆ alkyl group, and substituted C₁-C₆ alkyl group;

R₁₂ is selected from halogen, aryl, substituted aryl, amino, and amido; and

x is an integer from 1 to 6.

In some embodiments, the second terminus comprises a compound having the structure of Formula (9-C), or a pharmaceutically acceptable salt thereof:

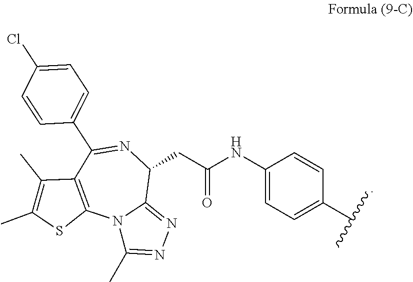 [SMILES: *c1ccc(NC(=O)C[C@H]2N=C(c3ccc(Cl)cc3)c3c(sc(C)c3C)-n3c(C)nnc32)cc1]

In some embodiments, the second terminus comprises a compound of Formula (10-A), or a pharmaceutically acceptable salt thereof:

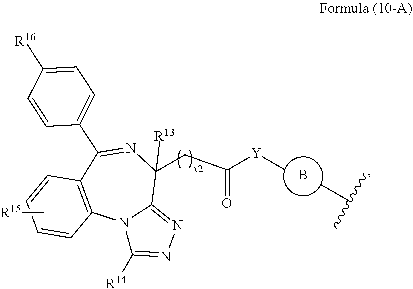

wherein,

Ring B is absent or 5-6-membered monocyclic aryl or heteroaryl or 4-8-membered heterocycle;

Y is —NH— or —O—;

R¹³ is selected from hydrogen or optionally substituted C₁-C₆ alkyl;

R¹⁴ and R¹⁵ are each independently selected from hydrogen, optionally substituted C₁₋₆ alkyl, C₁₋₆ haloalkyl, or C₁₋₆ hydroxyalkyl;

R¹⁶ is selected from hydrogen, halogen, —NO₂, —CN, optionally substituted aryl, optionally substituted C₁₋₆ alkyl, C₁₋₆ haloalkyl, or C₁₋₆ hydroxyalkyl;

or R¹⁶ is —NR^{A}R^{B}, wherein
R^{A} and R^{B} are each independently hydrogen, optionally substituted C₁₋₆ alkyl or C₁₋₆ heteroalkyl; and

x2 is an integer from 1-6.

In some embodiments, Ring B is 6-membered monocyclic aryl or heteroaryl. In some embodiments Ring B is phenyl. In some embodiments, Ring B is 6-membered monocyclic heteroaryl. In some embodiments, Ring B is pyridine or pyrimidine.

In some embodiments, Ring B is absent.

In some embodiments, Y is —NH—. In some embodiments, Y is —O—.

In some embodiments, R¹³ is hydrogen.

In some embodiments, R¹⁴ and R¹⁵ are each independently selected from optionally substituted C₁₋₆ alkyl, C₁₋₆ haloalkyl, or C₁₋₆ hydroxyalkyl. In some embodiments, R¹⁴ and R¹⁵ are each independently selected from optionally substituted C₁₋₆ alkyl. In some embodiments, In some embodiments, R¹⁴ and R¹⁵ are each independently methyl, ethyl, or propyl. In some embodiments, In some embodiments, R¹⁴ and R¹⁵ are each independently methyl.

In some embodiments, R¹⁶ is selected from hydrogen, halogen, optionally substituted C₁₋₆ alkyl, C₁₋₆ haloalkyl, or C₁₋₆ hydroxyalkyl. In some embodiments, R¹⁶ is bromo, chloro, or fluoro.

In some embodiments, R¹⁶ is —NR^{A}R^{B}, wherein R^{A} and R^{B} are each independently hydrogen, optionally substituted C₁₋₆ alkyl.

In some embodiments, x2 is an integer from 1-5, 1-4, 1-3, or 1-2. In some embodiments, x2 is 1. In some embodiments, x2 is 2.

In some embodiments, the second terminus comprises a compound having the structure of Formula (10-B), or a pharmaceutically acceptable salt thereof:

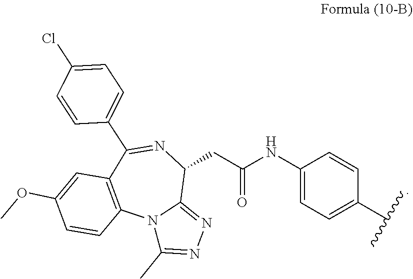 [SMILES: *c1ccc(NC(=O)C[C@H]2N=C(c3ccc(Cl)cc3)c3cc(OC)ccc3-n3c(C)nnc32)cc1]

In some embodiments, the second terminus comprises a compound having the structure of Formula (10-C), or a pharmaceutically acceptable salt thereof:

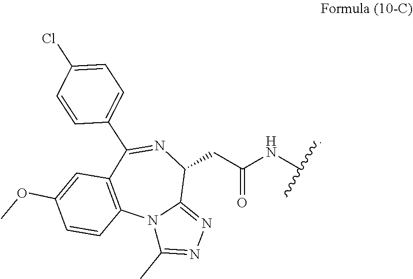 [SMILES: *NC(=O)C[C@H]1N=C(c2ccc(Cl)cc2)c2cc(OC)ccc2-n2c(C)nnc21]

In some embodiments, the second terminus comprises a compound having the structure of Formula (12-A), or a pharmaceutically acceptable salt thereof:

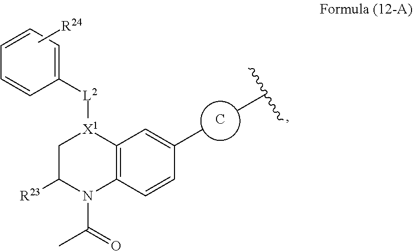

wherein;

Ring C is absent, monocyclic 6-membered aryl or heteroaryl, or 6-membered heterocycloalkylene;

X¹ is CH or N;

L² is —NR^{D}— or —CR^{D}H—

R²³ is C₁-C₆ alkyl or C₃-C₆ cycloalkyl; and

R²⁴ is halogen, alkyl, hydroxyalkyl, haloalkyl; optionally substituted C₁-C₆ alkyl, C₁-C₆ haloalkyl or C₁-C₆ hydroxyalkyl; and

R^{D} is hydrogen or C₁₋₃ alkyl.

In some embodiments, In some embodiments, Ring C is 6-membered monocyclic aryl or heteroaryl. In some embodiments Ring C is phenyl. In some embodiments, Ring C is 6-membered monocyclic heteroaryl. In some embodiments, Ring C is pyridine or pyrimidine. In some embodiments, Ring C is 6-membered heterocycloalkylene.

In some embodiments, In some embodiments, Ring C is

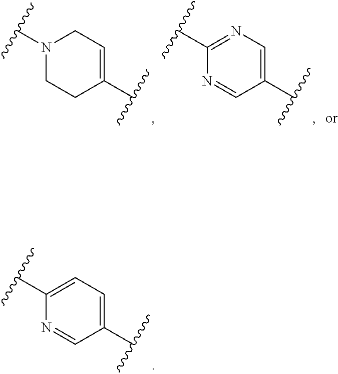 [SMILES: *C1=CCN(*)CC1 ; *c1cnc(*)nc1 ; *c1ccc(*)nc1]

In some embodiments, Ring C is absent.

In some embodiments, X¹ is CH. In some embodiments, X¹ is N.

In some embodiments, L² is —NR^{D}—. In some embodiments, L² is —NH—. In some embodiments, L² is —CR^{D}H. In some embodiments, L² is —CH₂—.

In some embodiments, R²³ is methyl, ethyl, or propyl. In some embodiments, R²³ is methyl. In some embodiments, R²³ is ethyl. In some embodiments, R²³ is propyl. In some embodiments, R²³ is cyclopropyl.

In some embodiments, R²⁴ is alkyl, hydroxyalkyl, haloalkyl; optionally substituted C₁-C₆ alkyl, C₁-C₆ haloalkyl or C₁-C₆ hydroxyalkyl. In some embodiments, R²⁴ is hydroxyalkyl. In some embodiments, R²⁴ is halogen. In some embodiments, R²⁴ is bromo, chloro, or fluoro. In some embodiments, R²⁴ is —CH₂OH.

In some embodiments, the second terminus comprises a compound having the structure of Formula (12-B), or a pharmaceutically acceptable salt thereof:

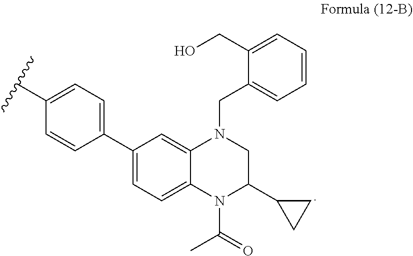 [SMILES: *c1ccc(-c2ccc3c(c2)N(Cc2ccccc2CO)CC(C2CC2)N3C(C)=O)cc1]

In some embodiments, the second terminus comprises a compound having the structure of Formula (12-B1), or a pharmaceutically acceptable salt thereof:

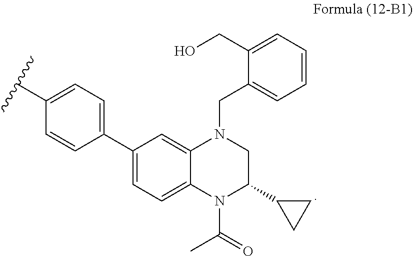 [SMILES: *c1ccc(-c2ccc3c(c2)N(Cc2ccccc2CO)C[C@H](C2CC2)N3C(C)=O)cc1]

In some embodiments, the second terminus comprises a compound having the structure of Formula (12-C), or a pharmaceutically acceptable salt thereof:

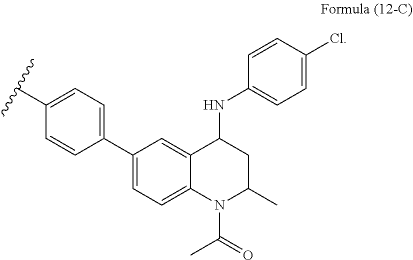 [SMILES: *c1ccc(-c2ccc3c(c2)C(Nc2ccc(Cl)cc2)CC(C)N3C(C)=O)cc1]

In some embodiments, the second terminus comprises a compound having the structure of Formula (12-C1), or a pharmaceutically acceptable salt thereof:

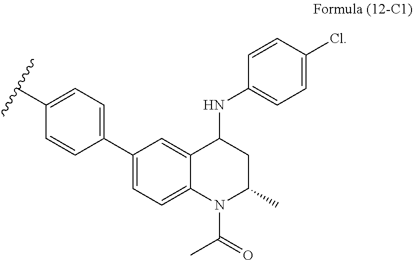 [SMILES: *c1ccc(-c2ccc3c(c2)C(Nc2ccc(Cl)cc2)C[C@H](C)N3C(C)=O)cc1]

In some embodiments, the second terminus comprises a compound having the structure of Formula (13-A), or a pharmaceutically acceptable salt thereof:

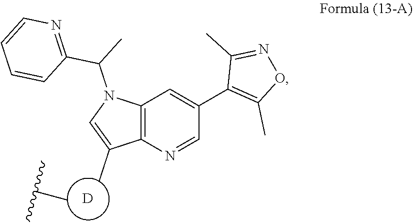

wherein;

Ring D is absent, or an optionally substituted phenyl, optionally 5 or 6-membered heteroaryl, or 6-membered heterocycloalkylene.

In some embodiments, Ring D is phenyl. In some embodiments, Ring D is absent.

In some embodiments, In some embodiments, Ring D is

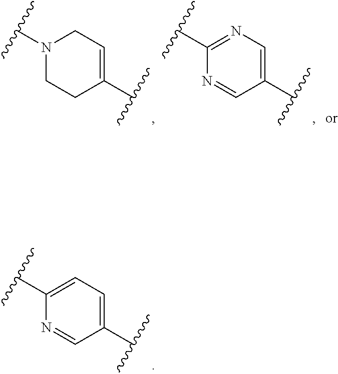 [SMILES: *C1=CCN(*)CC1 ; *c1cnc(*)nc1 ; *c1ccc(*)nc1]

In some embodiments, the second terminus comprises a compound having the structure of Formula (13-B), or a pharmaceutically acceptable salt thereof:

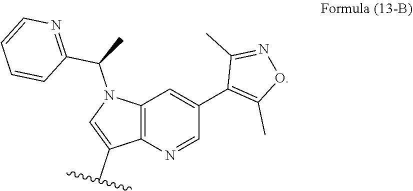 [SMILES: *c1cn([C@H](C)c2ccccn2)c2cc(-c3c(C)noc3C)cnc12]

In some embodiments, the second terminus comprises a compound having the structure of Formula (14-A), or a pharmaceutically acceptable salt thereof:

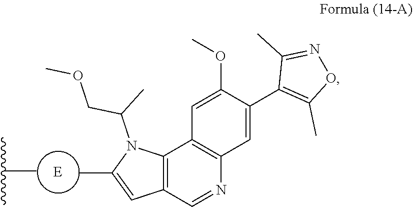

wherein;

Ring E is absent, or an optionally substituted phenyl, an optionally 5 or 6-membered heteroaryl, or 6-membered heterocycloalkylene.

In some embodiments, Ring E is phenyl. In some embodiments, Ring E is absent.

In some embodiments, In some embodiments, Ring E is

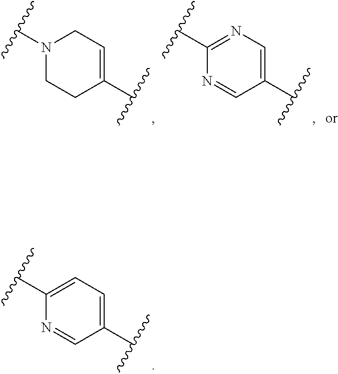 [SMILES: *C1=CCN(*)CC1 ; *c1cnc(*)nc1 ; *c1ccc(*)nc1]

In some embodiments, the second terminus comprises a compound having the structure of Formula (14-B), or a pharmaceutically acceptable salt thereof:

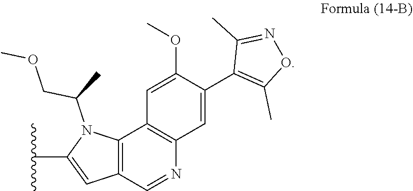 [SMILES: *c1cc2cnc3cc(-c4c(C)noc4C)c(OC)cc3c2n1[C@H](C)COC]

In some embodiments, the second terminus comprises a compound having the structure of Formula (15-A), or a pharmaceutically acceptable salt thereof:

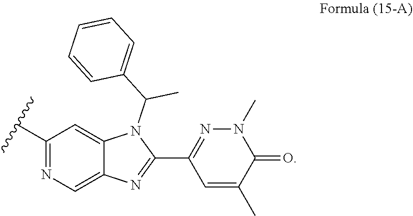 [SMILES: *c1cc2c(cn1)nc(-c1cc(C)c(=O)n(C)n1)n2C(C)c1ccccc1]

In some embodiments, the second terminus is:

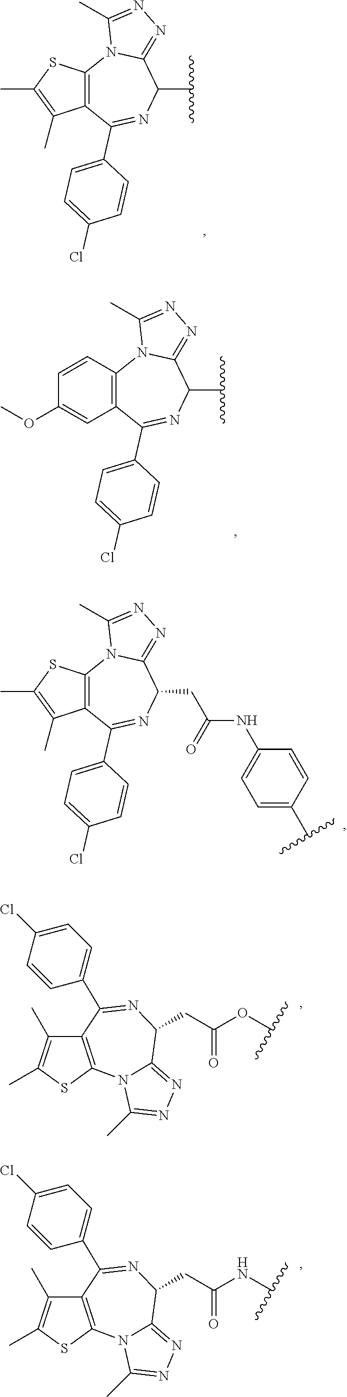 [SMILES: *C1N=C(c2ccc(Cl)cc2)c2c(sc(C)c2C)-n2c(C)nnc21 ; *C1N=C(c2ccc(Cl)cc2)c2cc(OC)ccc2-n2c(C)nnc21 ; *c1ccc(NC(=O)C[C@@H]2N=C(c3ccc(Cl)cc3)c3c(sc(C)c3C)-n3c(C)nnc32)cc1 ; *OC(=O)C[C@H]1N=C(c2ccc(Cl)cc2)c2c(sc(C)c2C)-n2c(C)nnc21 ; *NC(=O)C[C@H]1N=C(c2ccc(Cl)cc2)c2c(sc(C)c2C)-n2c(C)nnc21]

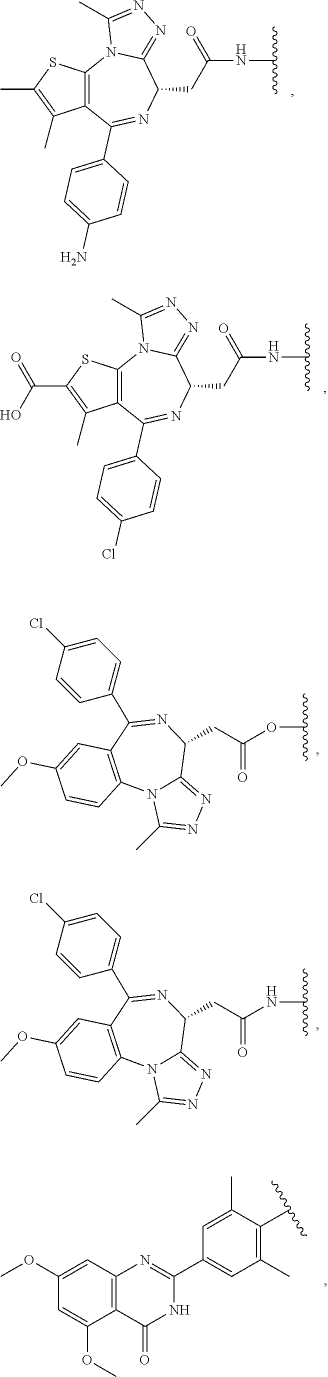 [SMILES: *NC(=O)C[C@@H]1N=C(c2ccc(N)cc2)c2c(sc(C)c2C)-n2c(C)nnc21 ; *NC(=O)C[C@@H]1N=C(c2ccc(Cl)cc2)c2c(sc(C(=O)O)c2C)-n2c(C)nnc21 ; *OC(=O)C[C@H]1N=C(c2ccc(Cl)cc2)c2cc(OC)ccc2-n2c(C)nnc21 ; *NC(=O)C[C@H]1N=C(c2ccc(Cl)cc2)c2cc(OC)ccc2-n2c(C)nnc21 ; *c1c(C)cc(-c2nc3cc(OC)cc(OC)c3c(=O)[nH]2)cc1C]

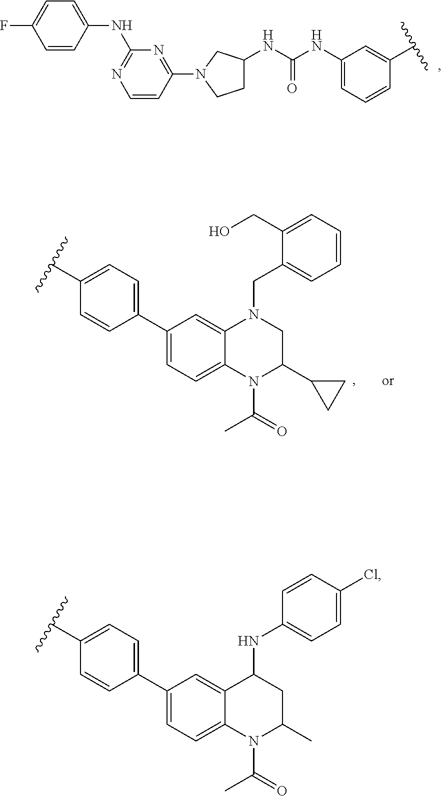 [SMILES: *c1cccc(NC(=O)NC2CCN(c3ccnc(Nc4ccc(F)cc4)n3)C2)c1 ; *c1ccc(-c2ccc3c(c2)N(Cc2ccccc2CO)CC(C2CC2)N3C(C)=O)cc1 ; *c1ccc(-c2ccc3c(c2)C(Nc2ccc(Cl)cc2)CC(C)N3C(C)=O)cc1]

or a pharmaceutically acceptable salt thereof.

In some embodiments, the protein binding moiety is selected from:

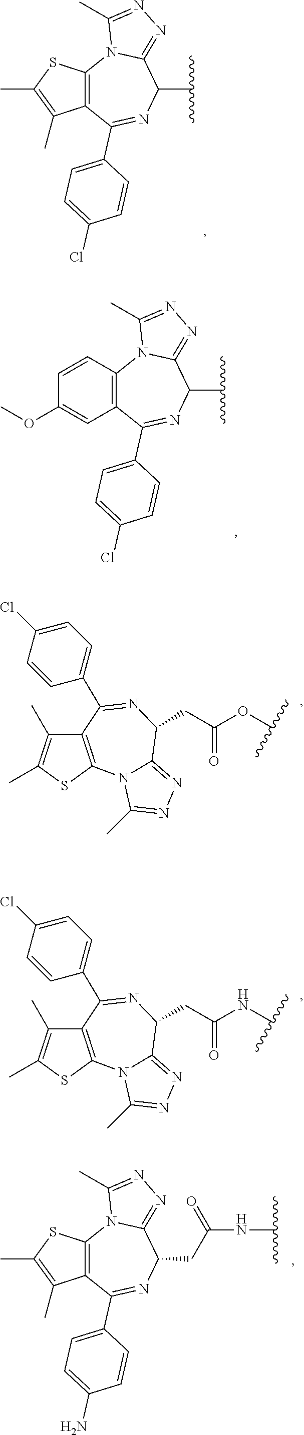 [SMILES: *C1N=C(c2ccc(Cl)cc2)c2c(sc(C)c2C)-n2c(C)nnc21 ; *C1N=C(c2ccc(Cl)cc2)c2cc(OC)ccc2-n2c(C)nnc21 ; *OC(=O)C[C@H]1N=C(c2ccc(Cl)cc2)c2c(sc(C)c2C)-n2c(C)nnc21 ; *NC(=O)C[C@H]1N=C(c2ccc(Cl)cc2)c2c(sc(C)c2C)-n2c(C)nnc21 ; *NC(=O)C[C@@H]1N=C(c2ccc(N)cc2)c2c(sc(C)c2C)-n2c(C)nnc21]

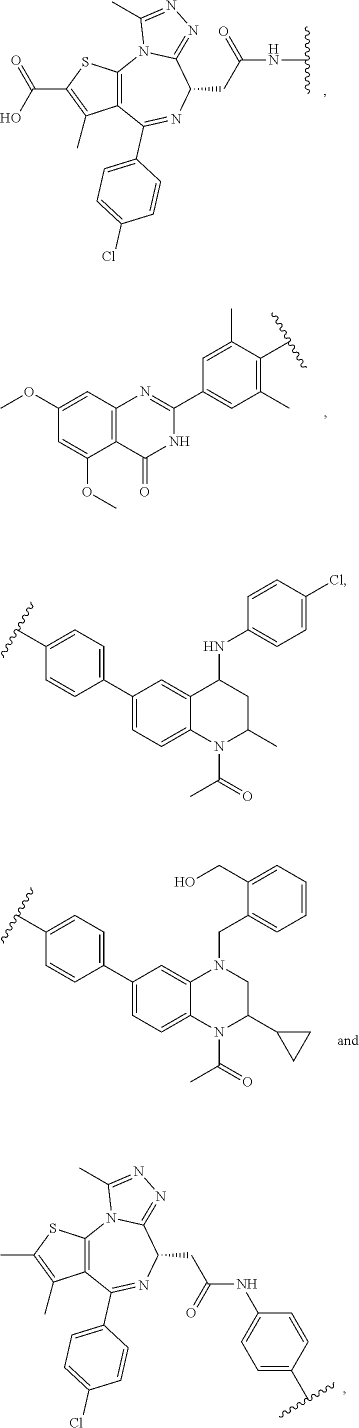 [SMILES: *NC(=O)C[C@@H]1N=C(c2ccc(Cl)cc2)c2c(sc(C(=O)O)c2C)-n2c(C)nnc21 ; *c1c(C)cc(-c2nc3cc(OC)cc(OC)c3c(=O)[nH]2)cc1C ; *c1ccc(-c2ccc3c(c2)C(Nc2ccc(Cl)cc2)CC(C)N3C(C)=O)cc1 ; *c1ccc(-c2ccc3c(c2)N(Cc2ccccc2CO)CC(C2CC2)N3C(C)=O)cc1 ; *c1ccc(NC(=O)C[C@@H]2N=C(c3ccc(Cl)cc3)c3c(sc(C)c3C)-n3c(C)nnc32)cc1]

or a pharmaceutically acceptable salt thereof.

In some embodiments, the protein binding moiety is

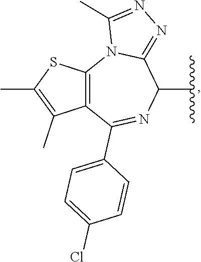 [SMILES: *C1N=C(c2ccc(Cl)cc2)c2c(sc(C)c2C)-n2c(C)nnc21]

or a pharmaceutically acceptable salt thereof.

### Linker

The oligomeric backbone contains a linker that connects the first terminus and the second terminus and brings the regulatory molecule in proximity to the target gene to modulate gene expression. In some embodiments, the terms “oligomeric backbone” and “backbone” denote a linker that connect the first terminus and the second terminus.

The length of the linker depends on the type of regulatory protein and also the target gene. In some embodiments, the linker has a length of less than about 50 Angstroms. In some embodiments, the linker has a length of about 15 to 40 Angstroms. In some embodiments, the linker comprises between 5 and 50 chain atoms. In some embodiments, the linker has a length of about 20 to 30 Angstroms.

In some embodiments, the linker comprises between 5 and 50 chain atoms.

In some embodiments, the linker comprises a multimer having 2 to 50 spacing moieties, wherein the spacing moiety is independently selected from the group consisting of —((CR^{3a}R^{3b})ₓ—O)_{y}—, —((CR^{3a}R^{3b})ₓ—NR^{4a})_{y}—, —((CR^{3a}R^{3b})ₓ—CH═CH—(CR^{3a}R^{3b})ₓ—O)_{y}—, optionally substituted —C₁₋₁₂ alkyl, optionally substituted C₂₋₁₀ alkenyl, optionally substituted C₂₋₁₀ alkynyl, optionally substituted C₆₋₁₀ arylene, optionally substituted C₃₋₇ cycloalkylene, optionally substituted 5- to 10-membered heteroarylene, optionally substituted 4- to 10-membered heterocycloalkylene, amino acid residue, —O—, —C(O)NR^{4a}—, —NR^{4a}C(O)—, —C(O)—, —NR¹—, —C(O)O—, —O—, —S—, —S(O)—, —SO₂—, —SO₂NR^{4a}—, —NR^{4a}SO₂—, and —P(O)OH—, and any combinations thereof; wherein

each x is independently 2-4;

each y is independently 1-10;

each R^{3a} and R^{3b} are independently selected from hydrogen, optionally substituted alkyl, optionally substituted alkenyl, optionally substituted alkynyl, optionally substituted alkoxy, optionally substituted amino, carboxyl, carboxyl ester, acyl, acyloxy, acyl amino, amino acyl, optionally substituted alkylamide, sulfonyl, optionally substituted thioalkoxy, optionally substituted aryl, optionally substituted heteroaryl, optionally substituted cycloalkyl, and optionally substituted heterocyclyl; and

each R^{4a} is independently a hydrogen or an optionally substituted C₁₋₆ alkyl.

In some embodiments, the linker comprise of —((CR^{3a}R^{3b})ₓ—O)_{y}—. In some embodiments, the linker comprises of —((CH₂)₂—O)_{y}—. In some embodiments, the linker comprises PEG. In some embodiments, the linker comprises between 1-20 PEG units. In some embodiments, the linker comprises 5, 6, 7, 8, 9, 10, 11, 12, 13, 14, 15, 16, 17, or 18 PEG units. In some embodiments, the linker comprises 6 PEG units. In some embodiments, the linker comprises 7 PEG units. In some embodiments, the linker comprises 8 PEG units. In some embodiments, the linker comprises 10 PEG units. In some embodiments, the linker comprises 15 PEG units.

In some embodiments, the oligomeric backbone comprises -(T¹-V¹)ₐ-(T²-V²)_{b}-(T³-V³)_{c}-(T⁴-V⁴)_{d}-(T⁵-V⁵)ₑ—,

wherein a, b, c, d and e are each independently 0 or 1, and where the sum of a, b, c, d and e is 1 to 5;

T¹, T², T³, T⁴ and T⁵ are each independently selected from an optionally substituted (C₁-C₁₂)alkylene, optionally substituted alkenylene, optionally substituted alkynylene, (EA)_{w}, (EDA)ₘ, (PEG)ₙ, (modified PEG)ₙ, (AA)ₚ, —(CR^{2a}OH)ₕ—, optionally substituted (C₆-C₁₀) arylene, optionally substituted C₃₋₇ cycloalkylene, optionally substituted 5- to 10 membered heteroarylene, optionally substituted 4- to 10-membered heterocycloalkylene, an acetal group, a disulfide, a hydrazine, a carbohydrate, a beta-lactam, and an ester,
w is an integer from 1 to 20;
m is an integer from 1 to 20;
n is an integer from 1 to 30;
p is an integer from 1 to 20;
h is an integer from 1 to 12;

EA has the following structure

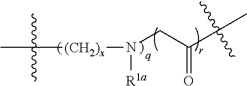

EDA has the following structure:

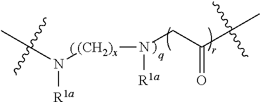

wherein each q is independently an integer from 1 to 6, each x is independently an integer from 1 to 4, and each r is independently 0 or 1;

(PEG)ₙ has the structure of —(CR^{2a}R^{2b}—CR^{2a}R^{2b}—O)ₙ—CR^{2a}R^{2b}—;

(modified PEG)ₙ has the structure of replacing at least one —(CR^{2a}R^{2b}—CR^{2a}R^{2b}—O)— in (PEG)ₙ with —(CH₂—CR^{2a}═CR^{2a}—CH₂—O)— or —(CR^{2a}R^{2b}—CR^{2a}R^{2b}—S)—;

AA is an amino acid residue;

V¹, V², V³, V⁴ and V⁵ are each independently selected from the group consisting of a bond, CO—, —NR^{1a}—, —CONR^{1a}—, —NR^{1a}CO—, —CONR^{1a}C₁₋₄ alkyl-, —NR^{1a}CO—C₁₋₄ alkyl-, —C(O)O—, —OC(O)—, —O—, —S—, —S(O)—, —SO₂—, —SO₂NR^{1a}—, —NR^{1a}SO₂— and —P(O)OH—;

each R^{1a} is independently hydrogen or and optionally substituted C₁₋₆ alkyl; and

each R^{2a} and R^{2b} are independently selected from hydrogen, alkyl, substituted alkyl, alkenyl, substituted alkenyl, alkynyl, substituted alkynyl, halogen, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In some embodiments, the a, b, c, d and e are each independently 0 or 1, where the sum of a, b, c, d and e is 1. In some embodiments, the a, b, c, d and e are each independently 0 or 1, where the sum of a, b, c, d and e is 2. In some embodiments, the a, b, c, d and e are each independently 0 or 1, where the sum of a, b, c, d and e is 3. In some embodiments, the a, b, c, d and e are each independently 0 or 1, where the sum of a, b, c, d and e is 4. In some embodiments, the a, b, c, d and e are each independently 0 or 1, where the sum of a, b, c, d and e is 5.

In some embodiments, n is 3-9. In some embodiments, n is 4-8. In some embodiments, n is 5 or 6.

In some embodiments, T¹, T², T³, and T⁴, and T⁵ are each independently selected from (C₁-C₁₂)alkyl, substituted (C₁-C₁₂)alkyl, (EA)_{w}, (EDA)ₘ, (PEG)ₙ, (modified PEG)ₙ, (AA)ₚ, —(CR^{2a}OH)ₕ—, phenyl, substituted phenyl, piperidin-4-amino (P4A), para-amino-benzyloxycarbonyl (PABC), meta-amino-benzyloxycarbonyl (MABC), para-amino-benzyloxy (PABO), meta-amino-benzyloxy (MABO), para-aminobenzyl, an acetal group, a disulfide, a hydrazine, a carbohydrate, a beta-lactam, an ester, (AA)ₚ-MABC-(AA)ₚ, (AA)ₚ-MABO-(AA)ₚ, (AA)ₚ-PABO-(AA)ₚ and (AA)ₚ-PABC-(AA)ₚ, In some embodiments, piperidin-4-amino (P4A) is

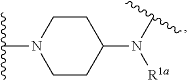

wherein R^{1a} is H or C₁₋₆ alkyl.

In some embodiments, T¹, T², T³, T⁴ and T⁵ are each independently selected from (C₁-C₁₂)alkyl, substituted (C₁-C₁₂)alkyl, (EA)_{w}, (EDA)ₘ, (PEG)ₙ, (modified PEG)ₙ, (AA)ₚ, —(CR^{2a}OH)ₕ—, optionally substituted (C₆-C₁₀) arylene, 4-10 membered heterocycloalkene, optionally substituted 5-10 membered heteroarylene. In some embodiments, EA has the following structure:

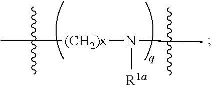

and

EDA has the following structure:

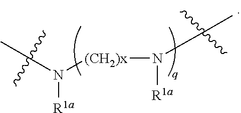

In some embodiments, x is 2-3 and q is 1-3 for EA and EDA. In some embodiments, R^{1a} is H or C₁₋₆ alkyl.

In some embodiments, T⁴ or V⁵ is an optionally substituted (C₆-C₁₀) arylene.

In some embodiments, T⁴ or T⁵ is phenylene or substituted phenylene. In some embodiments, T⁴ or T⁵ is phenylene or phenylene substituted with 1-3 substituents selected from —C₁₋₆ alkyl, halogen, OH or amine. In some embodiments, T⁴ or T⁵ is 5-10 membered heteroarylene or substituted heteroarylene. In some embodiments, T⁴ or T⁵ is 4-10 membered heterocylcylene or substituted heterocylcylene. In some embodiments, T⁴ or T⁵ is heteroarylene or heterocylcylene optionally substituted with 1-3 substituents selected from —C₁₋₆alkyl, halogen, OH or amine.

In some embodiments, T¹, T², T³, T⁴ and V⁵ and V¹, V², V³, V⁴ and V⁵ are selected from the following Table 2.

TABLE 2

| T¹ | V¹ | T² | V² | T³ | V³ | T⁴ | V⁴ | T⁵ | V⁵ |
|---|---|---|---|---|---|---|---|---|---|
| (C₁-C₁₂) | CONR^{1a} | (EA)_{w} | CO | (PEG)ₙ | NR¹¹CO | — | — | — | — |
| alkylene | | | | | | | | | |
| (C₁-C₁₂) | CONR^{1a} | (EA)_{w} | CO | (PEG)ₙ | O | arylene | NR¹¹CO | — | — |
| alkylene | | | | | | | | | |
| (C₁-C₁₂) | CONR^{1a} | (EA)_{w} | CO | (PEG)ₙ | O | Subst. | NR¹¹CO | — | — |
| alkylene | | | | | | arylene | | | |
| (C₁-C₁₂) | CONR^{1a} | (EA)_{w} | CO | (PEG)ₙ | O | NR¹¹CO | (C₁-C₁₂) | Subst. | NR¹¹CO |
| alkylene | | | | | | | alkyl | arylene | |
| (C₁-C₁₂) | CONR^{1a} | (EA)_{w} | CO | (C₁-C₁₂) | NR¹¹CO- | Subst. | NR¹¹ | — | — |
| alkylene | | | | alkyl | C₁₋₄ alkyl | arylene | | | |
| (C₁-C₁₂) | CONR^{1a} | (EA)_{w} | CO | (PEG)ₙ | O | Subst. | — | — | — |
| alkylene | | | | | | arylene | | | |
| (PEG)ₙ | CONR^{1a-} | — | — | — | — | — | — | — | — |
| | C₁₋₄ alkyl | | | | | | | | |
| (EA)_{w} | CO | (C₁-C₁₂) | CONR¹¹⁻ | — | — | — | — | — | — |
| | | alkyl | C₁₋₄ alkyl | | | | | | |
| (C₁-C₁₂) | CONR^{1a} | (EA)_{w} | CO | (PEG)ₙ | NR¹¹CO- | — | — | — | — |
| alkylene | | | | | C₁₋₄ alkyl | | | | |
| (EA)_{w} | CO | (PEG)ₙ | O | phenyl | NR¹¹CO- | — | — | — | — |
| | | | | | C₁₋₄ alkyl | | | | |
| (C₁-C₁₂) | CONR^{1a} | (PEG)ₙ | CO | — | — | — | — | — | — |
| alkylene | | | | | | | | | |
| (C₁-C₁₂) | CONR^{1a} | (EA)_{w} | CO | modifd. | O | arylene | NR¹¹CO | — | — |
| alkylene | | | | (PEG)ₙ | | | | | |

In some embodiments, the linker comprises

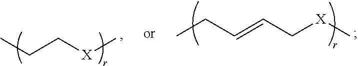

or any combinations thereof, wherein r is an integer between 1 and 10, preferably between 3 and 7; and X is O, S, or NR^{1a}. In some embodiments, X is O or NR^{1a}. In some embodiments, X is O.

In some embodiments, the linker comprise a

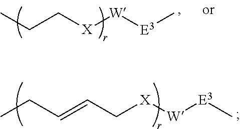

or any combinations thereof, wherein at least one —(CH₂—CH₂—O)— is replaced with —((CR^{1a}R^{1b})ₓ—CH═CH—(CR^{1a}R^{1b})ₓ—O)—, or any combinations thereof, W′ is absent, (CH₂)₁₋₅, —(CH₂)₁₋₅O, (CH₂)₁₋₅—C(O)NH—(CH₂)₁₋₅—O, (CH₂)₁₋₅—C(O)NH—(CH₂)₁₋₅, —(CH₂)₁₋₅NHC(O)—(CH₂)₁₋₅—O, or —(CH₂)₁₋₅—NHC(O)—(CH₂)₁₋₅—; E³ is an optionally substituted C₆₋₁₀ arylene group, optionally substituted 4-10 membered heterocycloalkylene, or optionally substituted 5-10 membered heteroarylene; X is O, S, or NH; each R^{1a} and R^{1b} are independently H or C₁₋₆ alkyl; r is an integer between 1 and 10; and x is an integer between 1 and 15. In some embodiments, X is O. In some embodiments, X is NH. In some embodiments, E³ is a C₆₋₁₀ arylene group optionally substituted with 1-3 substituents selected from —C₁₋₆ alkyl, halogen, OH or amine.

In some embodiments, E³ is a phenylene or substituted phenylene.

In some embodiments, the linker comprise a

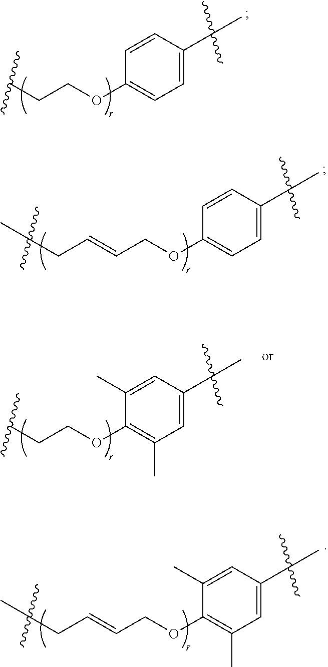

In some embodiments, the linker comprises —X(CH₂)ₘ(CH₂CH₂O)ₙ—, wherein X is —O—, —NH—, or —S—, wherein m is 0 or greater and n is at least 1.

In some embodiments, the linker comprises

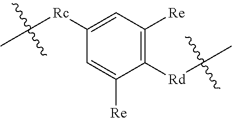

following the second terminus, wherein R_{c} is selected from a bond, —N(R^{1a})—, —O—, and —S—; R_{d} is selected from —N(R^{1a})—, —O—, and —S—; and Rₑ is independently selected from hydrogen and optionally substituted C₁₋₆ alkyl.

In some embodiments, the linker comprises one or more structures selected from

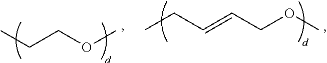

—C₁₋₁₂ alkyl, arylene, cycloalkylene, heteroarylene, heterocycloalkylene, —O—, —C(O)NR^{1a}—, —C(O)—, —NR^{1a}—, —(CH₂CH₂CH₂O)_{y}—, and —(CH₂CH₂CH₂NR^{1a})_{y}—, wherein each d and y are independently 1-10, and each R^{1a} is independently hydrogen or C₁₋₆ alkyl. In some embodiments, d is 4-8.

In some embodiments, the linker comprises

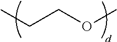

and each d is independently 3-7. In some embodiments, d is 4-6.

In some embodiments, the linker comprises —N(R^{1a})(CH₂)ₓN(R^{1b})(CH₂)ₓN—, wherein R^{1a} and R^{1b} are each independently selected from hydrogen or optionally substituted C₁-C₆ alkyl; and each x is independently an integer in the range of 1-6.

In some embodiments, the linker comprises the linker comprises —(CH₂—C(O)N(R″)—(CH₂)_{q}—N(R′)—(CH₂)_{q}—N(R″)C(O)—(CH₂)ₓ—C(O)N(R″)-A²-, —(CH₂)ₓ—C(O)N(R″)—(CH₂CH₂O)_{y}(CH₂)ₓ—C(O)N(R″)-A²-, —C(O)N(R″)—(CH₂)_{q}—N(R′)—(CH₂)_{q}—N(R″)C(O)—(CH₂)ₓ-A²-, —(CH₂)ₓO—(CH₂CH₂O)_{y}—(CH₂)ₓ—N(R″)C(O)—(CH₂)ₓ-A²-, or —N(R″)C(O)—(CH₂)—C(O)N(R″)—(CH₂)ₓ—O(CH₂CH₂O)_{y}(CH₂)ₓ-A²-; wherein R′ is methyl; R″ is hydrogen; each x and y are independently an integer from 1 to 10; each q is independently an integer from 2 to 10; and each A² is independently selected from a bond, an optionally substituted C₁₋₁₂ alkyl, an optionally substituted C₆₋₁₀ arylene, optionally substituted C₃₋₇ cycloalkylene, optionally substituted 5- to 10-membered heteroarylene, and optionally substituted 4- to 10-membered heterocycloalkylene.

In some embodiments, the linker is joined with the first terminus with a group selected from —CO—, —NR^{1a}—, —CONR^{1a}—, —NR^{1a}CO—, —CONR^{1a}C₁₋₄alkyl-, —NR^{1a}CO—C₁₋₄alkyl-, —C(O)O—, —OC(O)—, —O—, —S—, —S(O)—, —SO₂—, —SO₂NR^{1a}—, —NR^{1a}SO₂—, —P(O)OH—, —((CH₂)ₓ—O)—, —((CH₂)_{y}—NR^{1a})—, optionally substituted —C₁₋₁₂ alkylene, optionally substituted C₂₋₁₀ alkenylene, optionally substituted C₂₋₁₀ alkynylene, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene; wherein each x and y are independently 1-4, and each R^{1a} is independently a hydrogen or optionally substituted C₁₋₆ alkyl.

In some embodiments, the linker is joined with the first terminus with a group selected from —CO—, —NR^{1a}—, C₁₋₁₂ alkyl, —CONR^{1a}—, and —NR^{1a}CO—; wherein each R^{1a} is independently a hydrogen or optionally substituted C₁₋₆ alkyl or optionally substituted —C₁₋₁₂ alkylene, optionally substituted C₂₋₁₀ alkenylene, optionally substituted C₂₋₁₀ alkynylene, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene.

In some embodiments, In some embodiments, the linker is joined with the first terminus with a group selected from —CO—, —NR^{1a}—, C₁₋₁₂ alkyl, —CONR^{1a}—, and —NR^{1a}CO—; wherein each R^{1a} is independently a hydrogen or optionally substituted C₁₋₆ alkyl.

In some embodiments, the linker is joined with the first terminus with a group selected from —CO—, —NR^{1a}—, —CONR^{1a}—, —NR^{1a}CO—, —CONR^{1a}C₁₋₄alkyl-, —NR^{1a}CO—C₁₋₄alkyl-, —C(O)O—, —OC(O)—, —O—, —S—, —S(O)—, —SO₂—, —SO₂NR^{1a}—, —NR¹SO₂—, —P(O)OH—, —((CH₂)ₓ—O)—, —((CH₂)_{y}—NR^{1a})—, optionally substituted —C₁₋₁₂ alkylene, optionally substituted C₂₋₁₀ alkenylene, optionally substituted C₂₋₁₀ alkynylene, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene, wherein each x is independently 1-4, each y is independently 1-4, and each R^{1a} is independently a hydrogen or optionally substituted C₁₋₆ alkyl.

In some embodiments, the linker is joined with the first terminus with a group selected from —CO—, —NR^{1a}—, C₁₋₁₂ alkyl, —CONR^{1a}—, and —NR^{1a}CO—.

In some embodiments, the linker is joined with second terminus with a group selected from —CO—, —NR^{1a}—, —CONR^{1a}—, —NR^{1a}CO—, —CONR^{1a}C₁₋₄alkyl-, —NR^{1a}CO—C₁₋₄alkyl-, —C(O)O—, —OC(O)—, —O—, —S—, —S(O)—, —SO₂—, —SO₂NR^{1a}—, —NR¹SO₂—, —P(O)OH—, —((CH₂)ₓ—O)—, —((CH₂)_{y}—NR^{1a})—, optionally substituted —C₁₋₁₂ alkylene, optionally substituted C₂₋₁₀ alkenylene, optionally substituted C₂₋₁₀ alkynylene, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene, wherein each x is independently 1-4, each y is independently 1-4, and each R^{1a} is independently a hydrogen or optionally substituted C₁₋₆ alkyl.

In some embodiments, the linker is joined with second terminus with a group selected from —CO—, —NR^{1a}—, —CONR^{1a}—, —NR^{1a}CO—, —((CH₂)ₓ—O)—, —((CH₂)_{y}—NR^{1a})—, —O—, optionally substituted —C₁₋₁₂ alkyl, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene, wherein each x is independently 1-4, each y is independently 1-4, and each R¹ is independently a hydrogen or optionally substituted C₁₋₆ alkyl.

In some embodiments, the linker is joined with second terminus with a group selected from optionally substituted 4- to 10-membered heterocycloalkylene.

### Cell-Penetrating Ligand

In certain embodiments, the compounds comprise a cell-penetrating ligand moiety.

In certain embodiments, the cell-penetrating ligand moiety is a polypeptide.

In certain embodiments, the cell-penetrating ligand moiety is a polypeptide containing fewer than 30 amino acid residues.

In certain embodiments, the polypeptide is chosen from any one of SEQ ID NO. 1 to SEQ ID NO. 37, inclusive.

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

In some embodiments, non-limiting examples of the transcription modulator compounds described herein are presented in Table 3.

TABLE 3
Compounds of the disclosure.

| Cpd. | Structure |
|---|---|
| 1 | 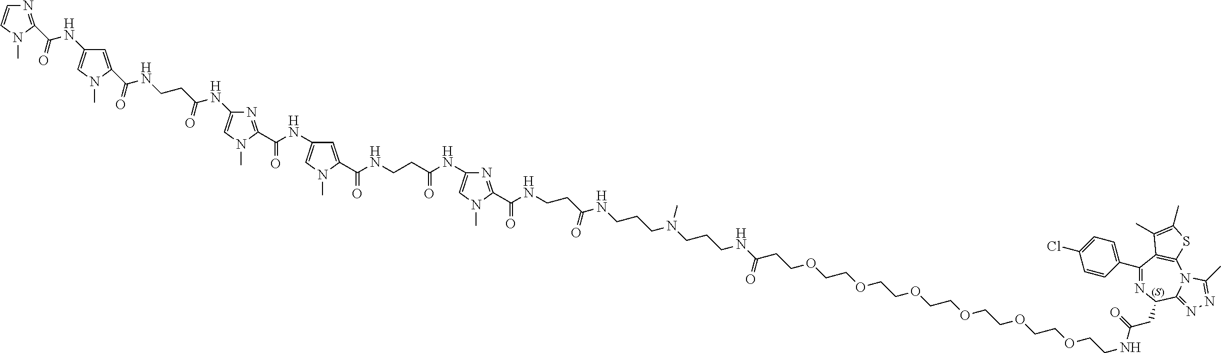 |
| 2 | 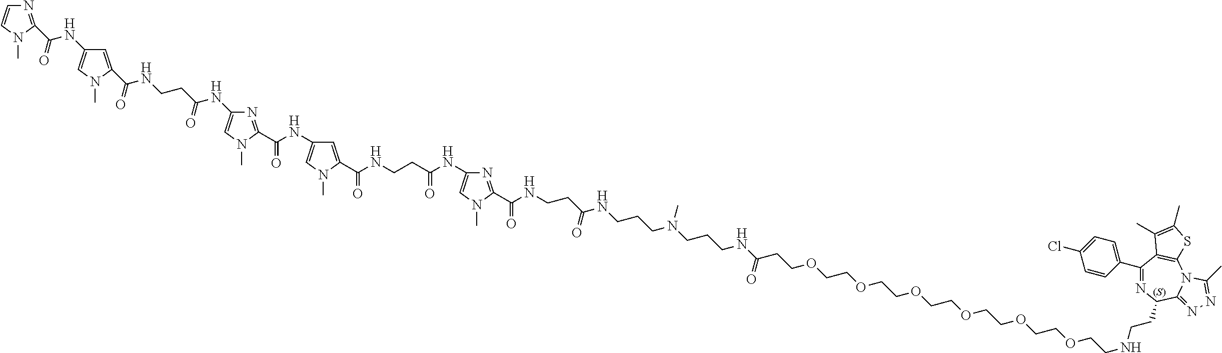 |
| 3 | 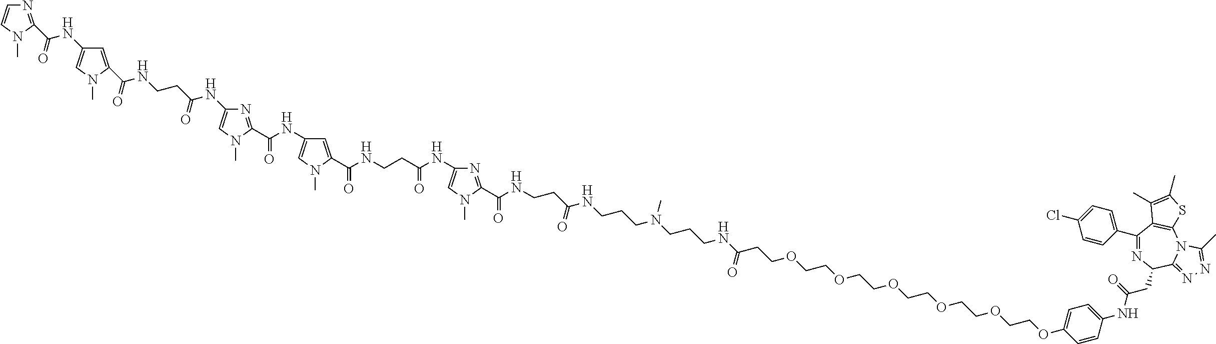 [SMILES: Cc1sc2c(c1C)C(c1ccc(Cl)cc1)=N[C@@H](CC(=O)Nc1ccc(OCCOCCOCCOCCOCCOCCC(=O)NCCCN(C)CCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 4 | 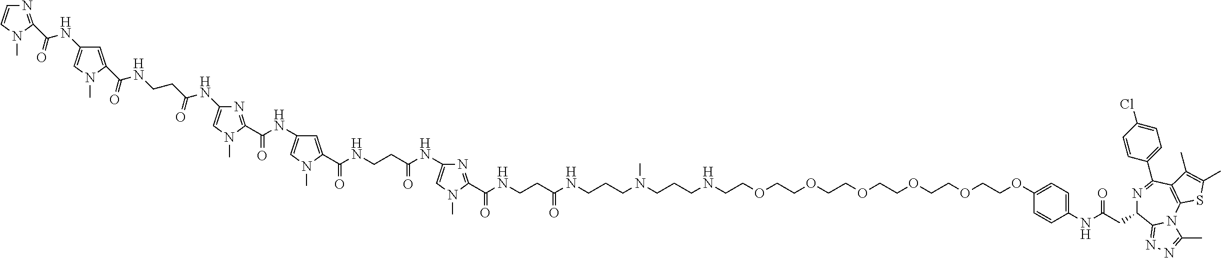 [SMILES: Cc1sc2c(c1C)C(c1ccc(Cl)cc1)=N[C@@H](CC(=O)Nc1ccc(OCCOCCOCCOCCOCCOCCNCCCN(C)CCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 5 | 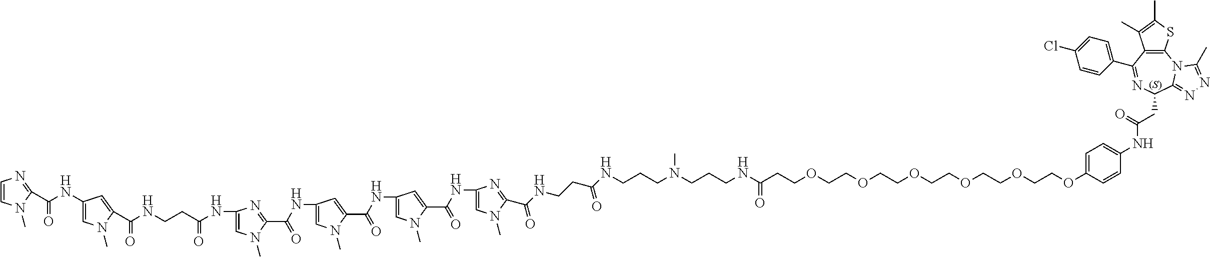 |
| 6 | 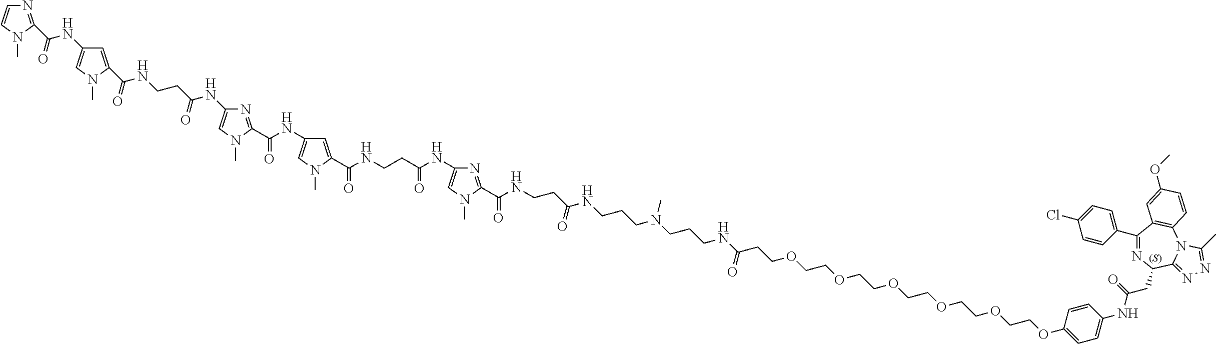 |
| 7 | 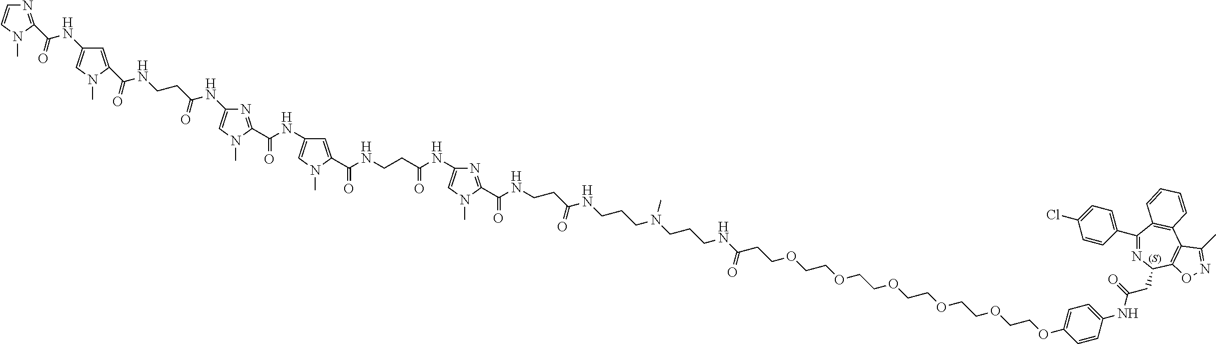 |
| 8 | 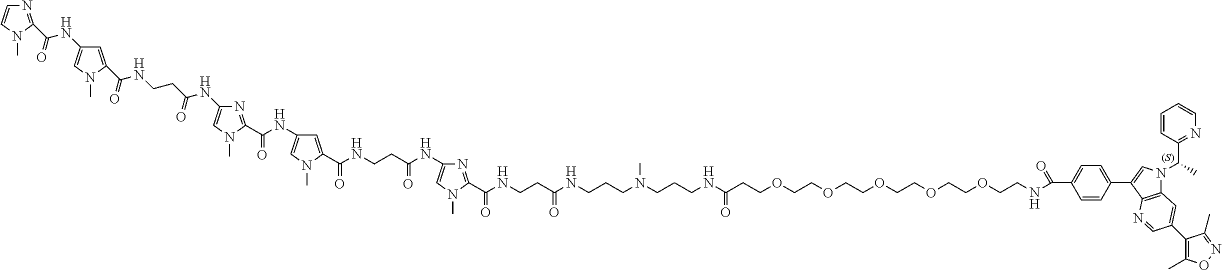 |
| 9 | 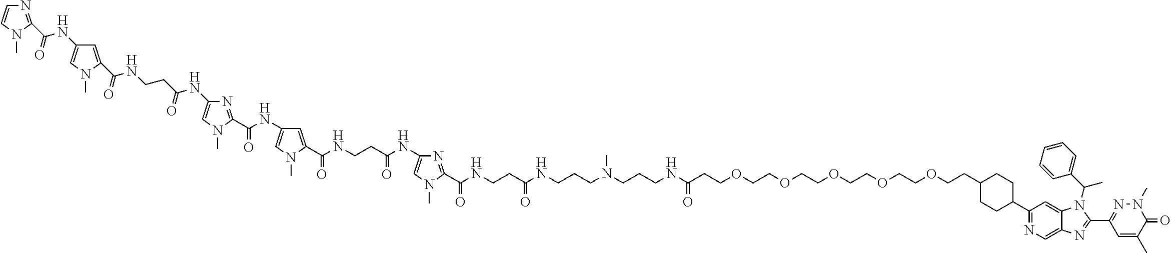 [SMILES: Cc1cc(-c2nc3cnc(C4CCC(CCOCCOCCOCCOCCOCCC(=O)NCCCN(C)CCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)cc3n2C(C)c2ccccc2)nn(C)c1=O] |
| 10 | 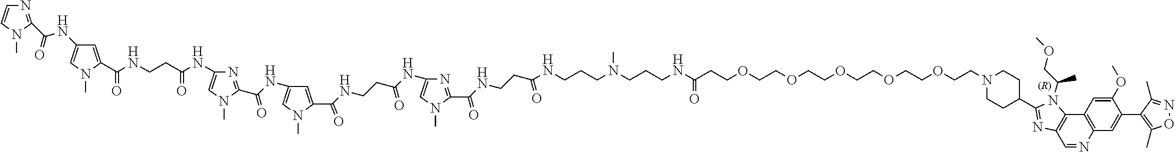 |
| 11 | 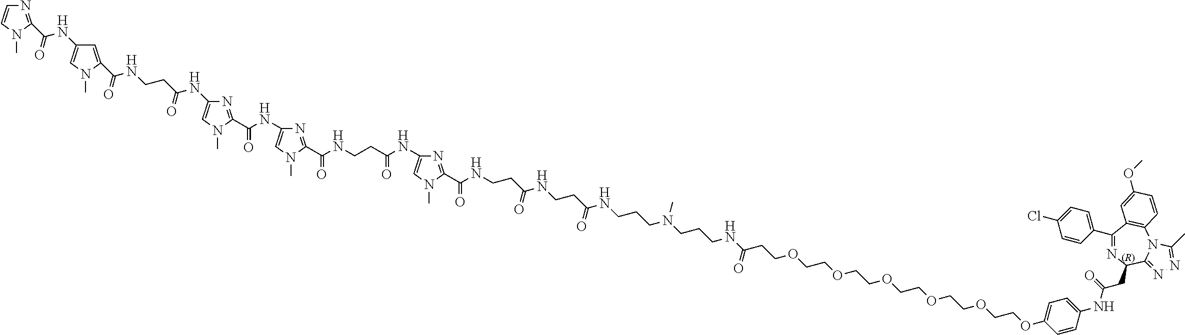 |
| 12 | 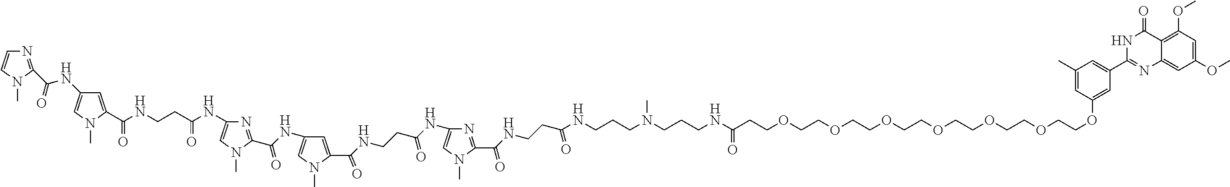 [SMILES: COc1cc(OC)c2c(=O)[nH]c(-c3cc(C)cc(OCCOCCOCCOCCOCCOCCOCCC(=O)NCCCN(C)CCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)c3)nc2c1] |
| 13 | 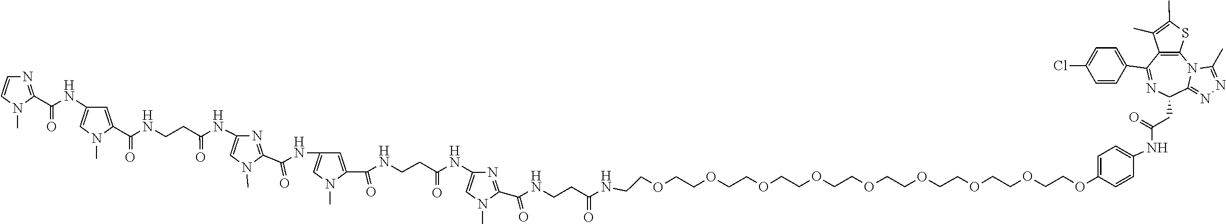 [SMILES: Cc1sc2c(c1C)C(c1ccc(Cl)cc1)=N[C@@H](CC(=O)Nc1ccc(OCCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 14 | 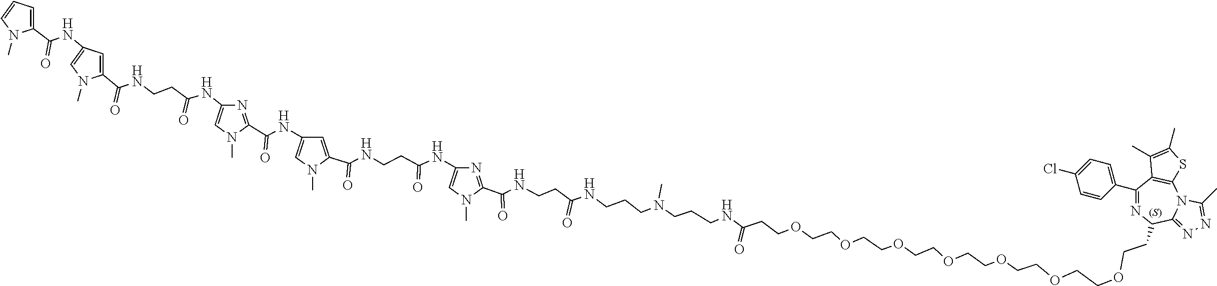 |
| 15 | 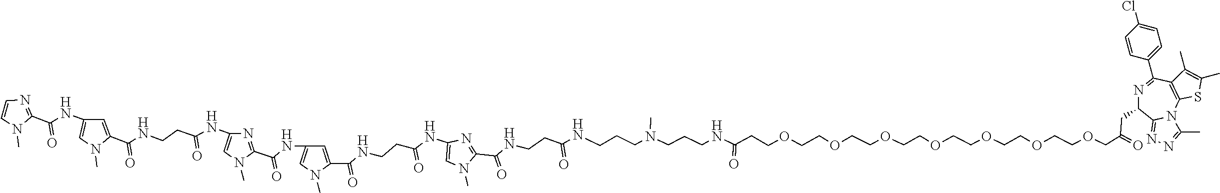 [SMILES: Cc1sc2c(c1C)C(c1ccc(Cl)cc1)=N[C@@H](CC(=O)COCCOCCOCCOCCOCCOCCOCCC(=O)NCCCN(C)CCCNC(=O)CCNC(=O)c1nc(NC(=O)CCNC(=O)c3cc(NC(=O)c4nc(NC(=O)CCNC(=O)c5cc(NC(=O)c6nccn6C)cn5C)cn4C)cn3C)cn1C)c1nnc(C)n1-2] |
| 16 | 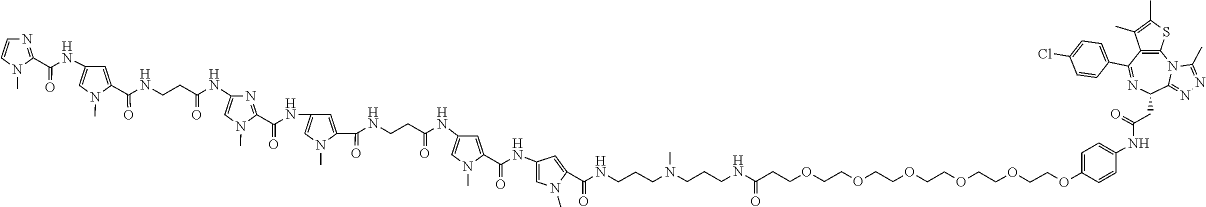 [SMILES: Cc1sc2c(c1C)C(c1ccc(Cl)cc1)=N[C@@H](CC(=O)Nc1ccc(OCCOCCOCCOCCOCCOCCC(=O)NCCCN(C)CCCNC(=O)c3cc(NC(=O)c4cc(NC(=O)CCNC(=O)c5cc(NC(=O)c6nc(NC(=O)CCNC(=O)c7cc(NC(=O)c8nccn8C)cn7C)cn6C)cn5C)cn4C)cn3C)cc1)c1nnc(C)n1-2] |
| 17 | 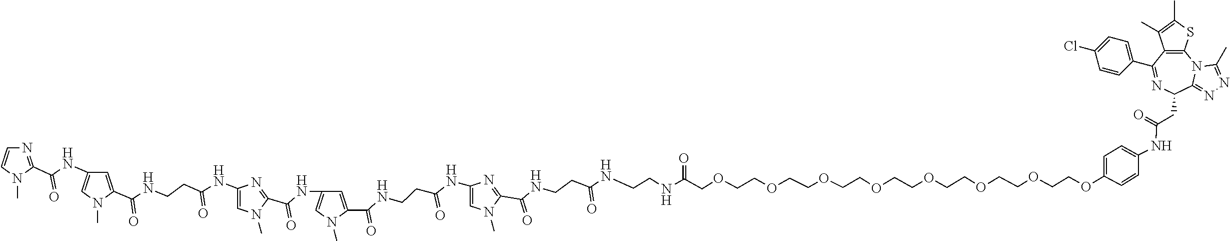 [SMILES: Cc1sc2c(c1C)C(c1ccc(Cl)cc1)=N[C@@H](CC(=O)Nc1ccc(OCCOCCOCCOCCOCCOCCOCCOCC(=O)NCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 18 | 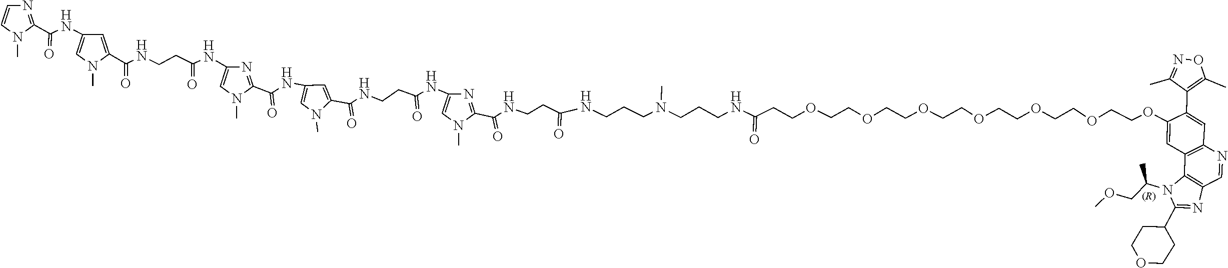 |
| 19 | 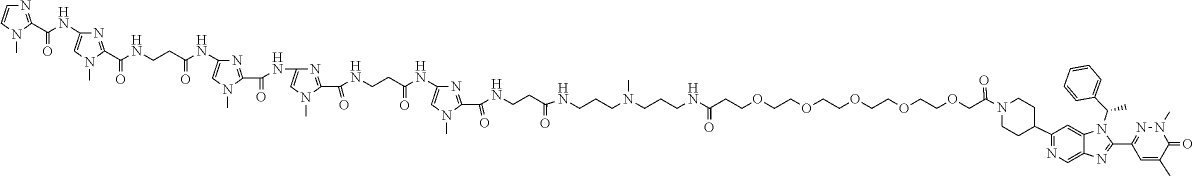 [SMILES: Cc1cc(-c2nc3cnc(C4CCN(C(=O)COCCOCCOCCOCCOCCC(=O)NCCCN(C)CCCNC(=O)CCNC(=O)c5nc(NC(=O)CCNC(=O)c6nc(NC(=O)c7nc(NC(=O)CCNC(=O)c8nc(NC(=O)c9nccn9C)cn8C)cn7C)cn6C)cn5C)CC4)cc3n2[C@@H](C)c2ccccc2)nn(C)c1=O] |
| 20 | 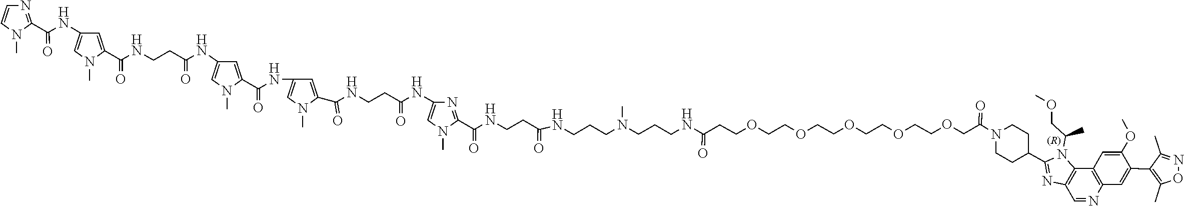 |
| 21 | 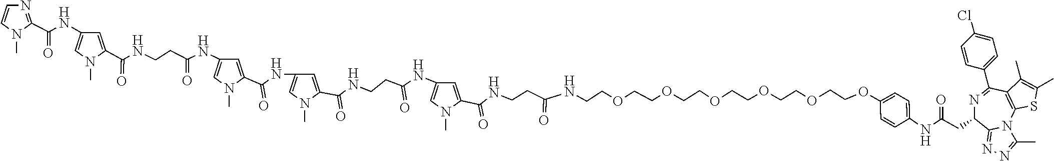 [SMILES: Cc1sc2c(c1C)C(c1ccc(Cl)cc1)=N[C@@H](CC(=O)Nc1ccc(OCCOCCOCCOCCOCCOCCNC(=O)CCNC(=O)c3cc(NC(=O)CCNC(=O)c4cc(NC(=O)c5cc(NC(=O)CCNC(=O)c6cc(NC(=O)c7nccn7C)cn6C)cn5C)cn4C)cn3C)cc1)c1nnc(C)n1-2] |
| 22 | 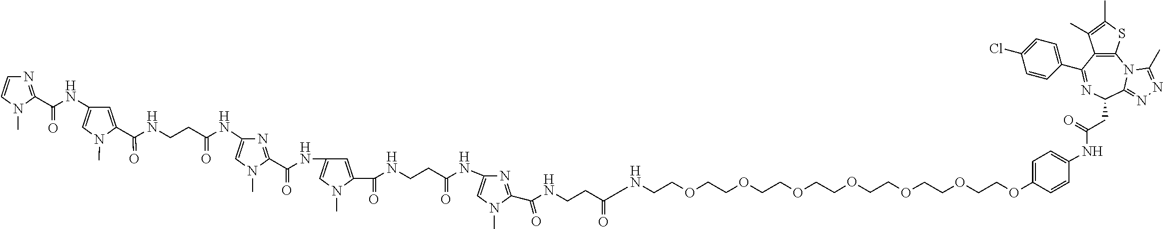 [SMILES: Cc1sc2c(c1C)C(c1ccc(Cl)cc1)=N[C@@H](CC(=O)Nc1ccc(OCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 23 | 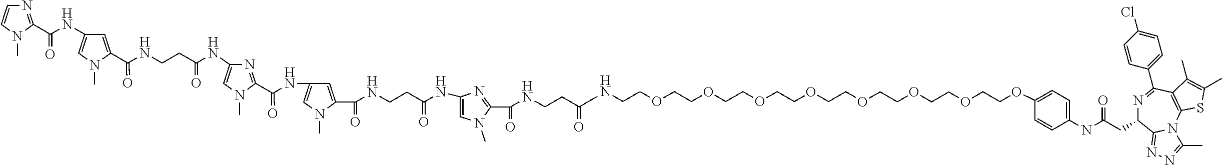 |
| 24 | 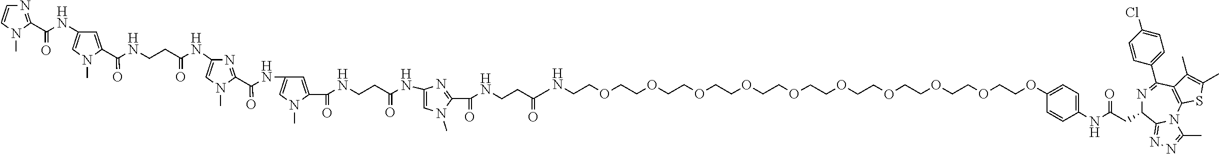 [SMILES: Cc1sc2c(c1C)C(c1ccc(Cl)cc1)=N[C@@H](CC(=O)Nc1ccc(OCCOCCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 25 | 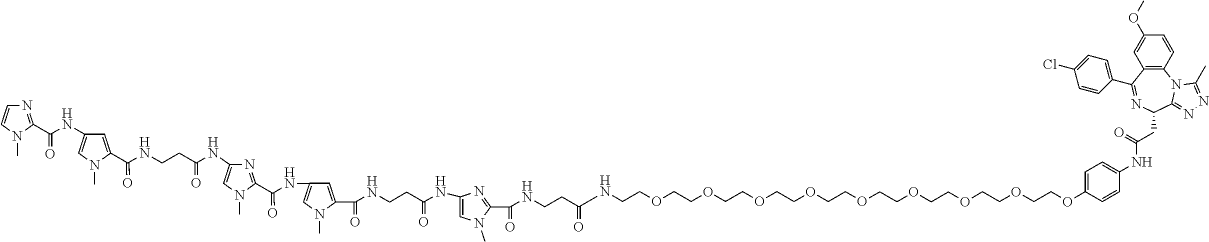 [SMILES: COc1ccc2c(c1)C(c1ccc(Cl)cc1)=N[C@@H](CC(=O)Nc1ccc(OCCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 26 | 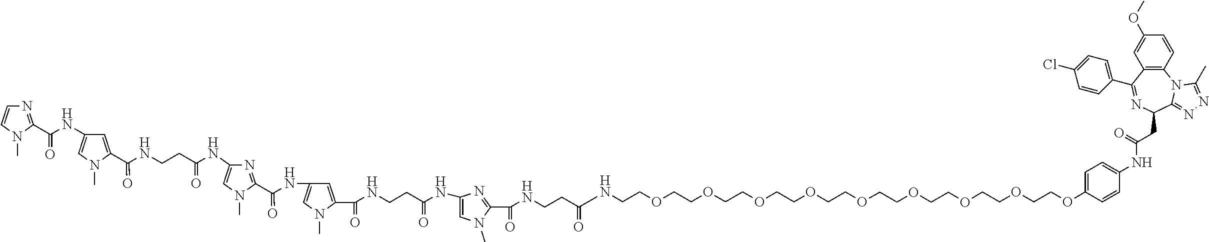 [SMILES: COc1ccc2c(c1)C(c1ccc(Cl)cc1)=N[C@H](CC(=O)Nc1ccc(OCCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 27 | 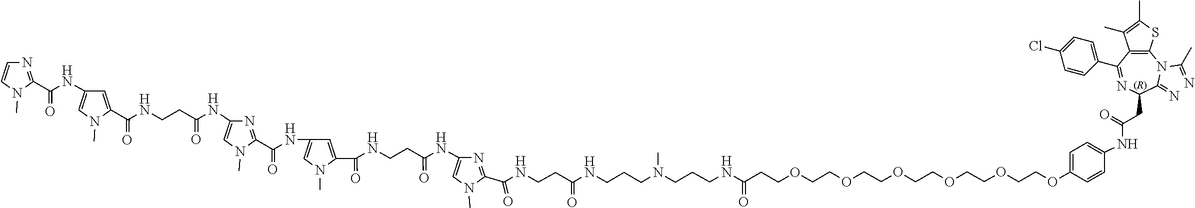 |
| 28 | 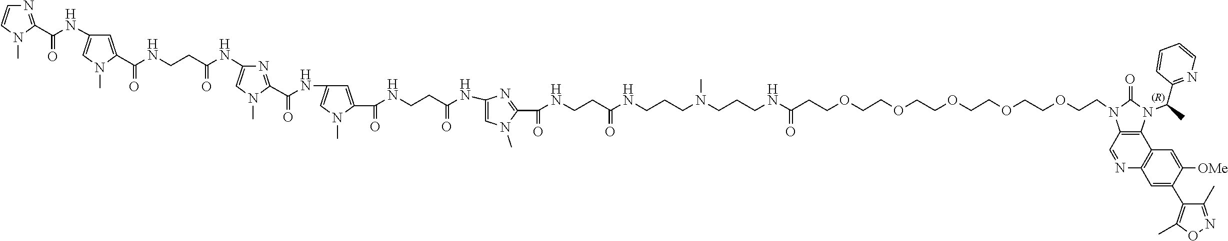 |
| 29 | 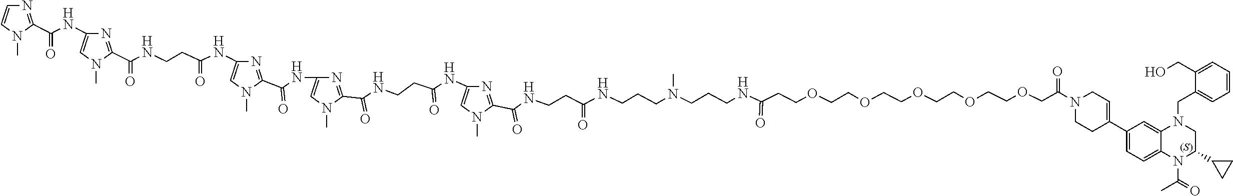 |
| 30 | 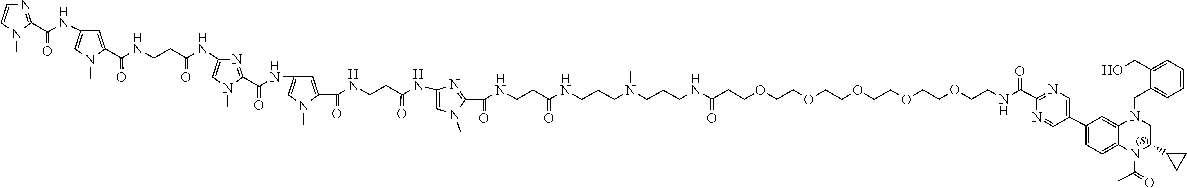 |
| 31 | 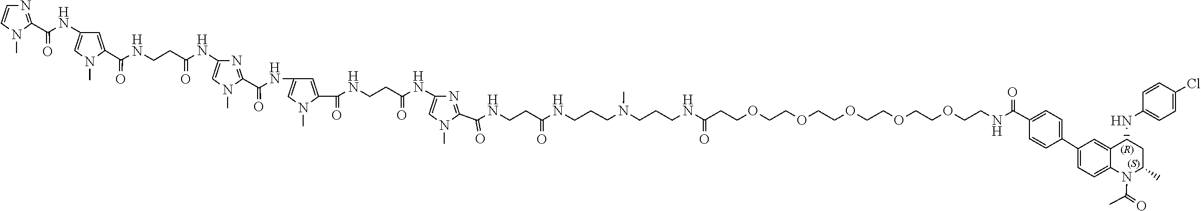 |
| 32 | 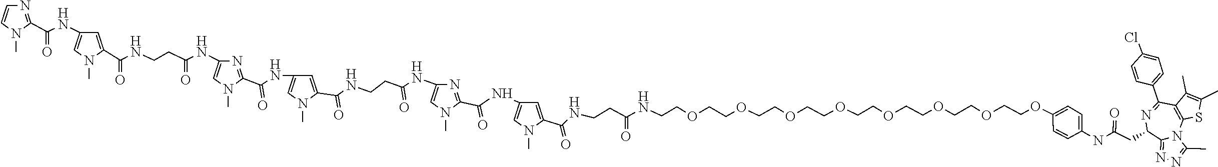 |
| 33 | 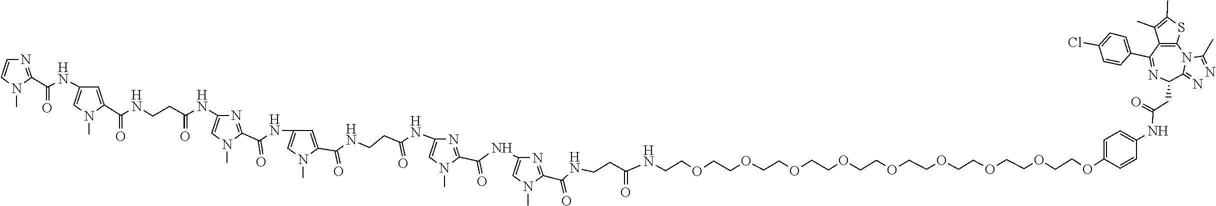 [SMILES: Cc1sc2c(c1C)C(c1ccc(Cl)cc1)=N[C@@H](CC(=O)Nc1ccc(OCCOCCOCCOCCOCCOCCOCCOCCOCCNC(=O)CCNC(=O)c3nc(NC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cn3C)cc1)c1nnc(C)n1-2] |
| 34 | 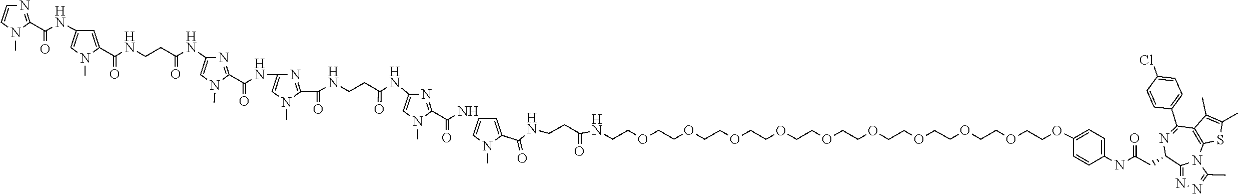 |
| 35 | 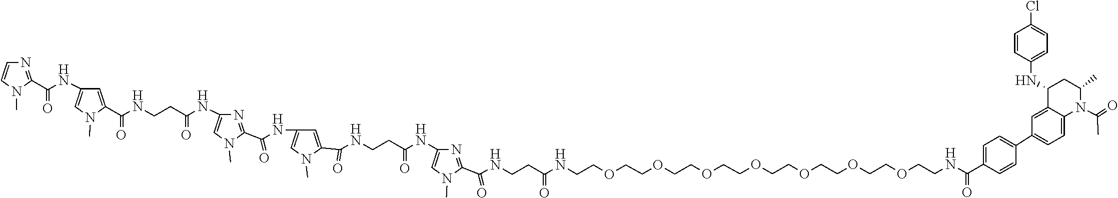 [SMILES: CC(=O)N1c2ccc(-c3ccc(C(=O)NCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cc3)cc2[C@H](Nc2ccc(Cl)cc2)C[C@@H]1C] |
| 36 | 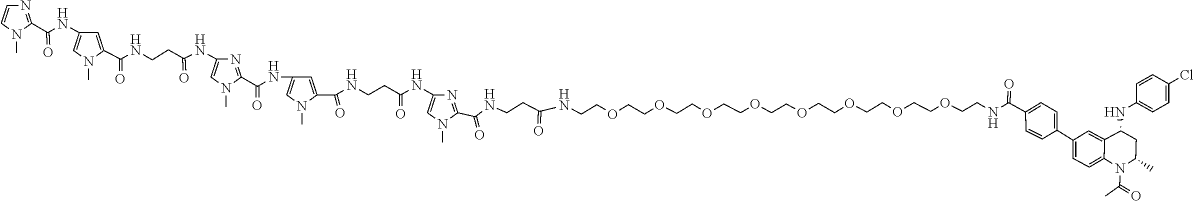 [SMILES: CC(=O)N1c2ccc(-c3ccc(C(=O)NCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cc3)cc2[C@H](Nc2ccc(Cl)cc2)C[C@@H]1C] |
| 37 | 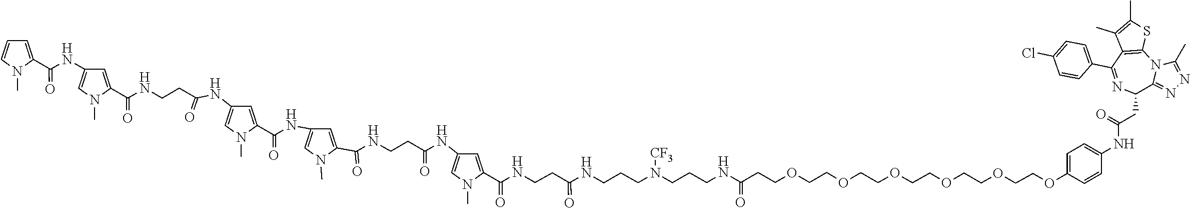 [SMILES: Cc1sc2c(c1C)C(c1ccc(Cl)cc1)=N[C@@H](CC(=O)Nc1ccc(OCCOCCOCCOCCOCCOCCC(=O)NCCCN(CCCNC(=O)CCNC(=O)c3cc(NC(=O)CCNC(=O)c4cc(NC(=O)c5cc(NC(=O)CCNC(=O)c6cc(NC(=O)c7cccn7C)cn6C)cn5C)cn4C)cn3C)C(F)(F)F)cc1)c1nnc(C)n1-2] |
| 38 | 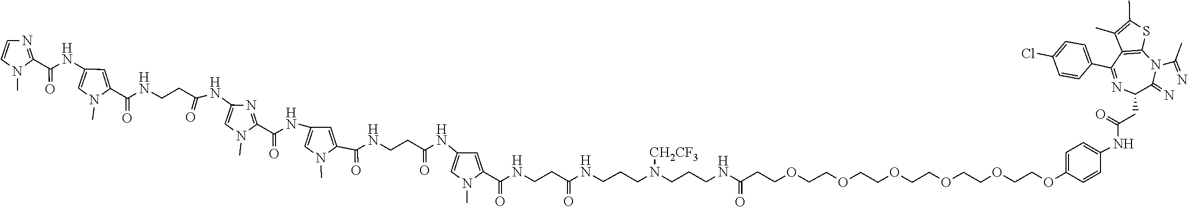 |
| 39 | 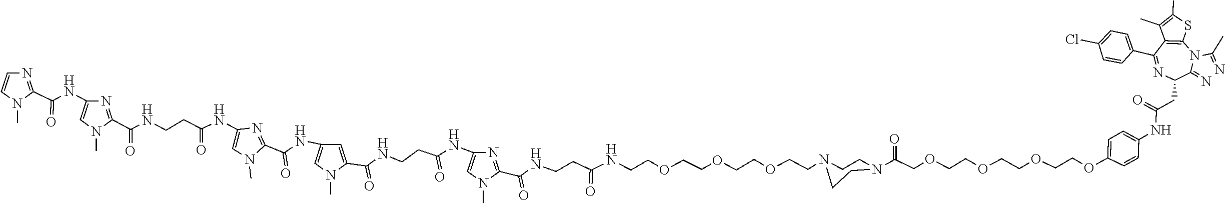 [SMILES: Cc1sc2c(c1C)C(c1ccc(Cl)cc1)=N[C@@H](CC(=O)Nc1ccc(OCCOCCOCCOCC(=O)N3CCN(CCOCCOCCOCCNC(=O)CCNC(=O)c4nc(NC(=O)CCNC(=O)c5cc(NC(=O)c6nc(NC(=O)CCNC(=O)c7nc(NC(=O)c8nccn8C)cn7C)cn6C)cn5C)cn4C)CC3)cc1)c1nnc(C)n1-2] |
| 40 | 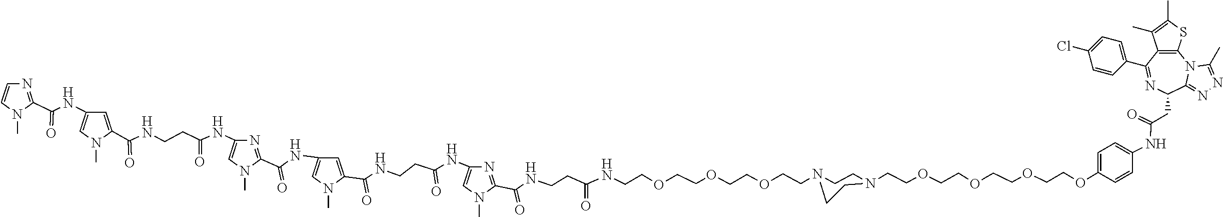 [SMILES: Cc1sc2c(c1C)C(c1ccc(Cl)cc1)=N[C@@H](CC(=O)Nc1ccc(OCCOCCOCCOCCN3CCN(CCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)cc1)c1nnc(C)n1-2] |
| 41 | 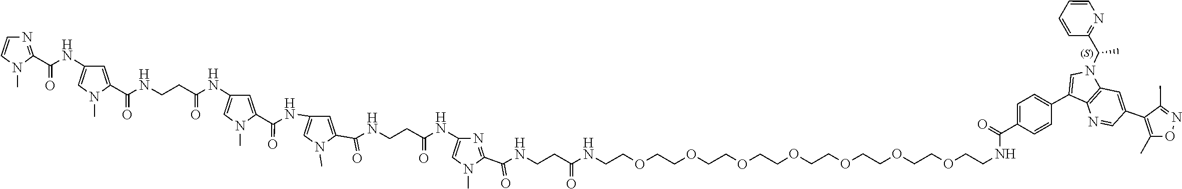 |
| 42 | 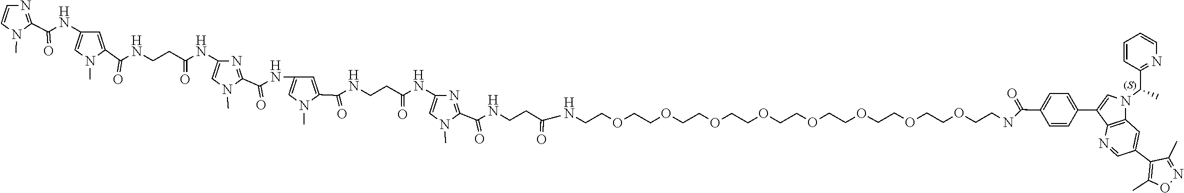 |
| 43 | 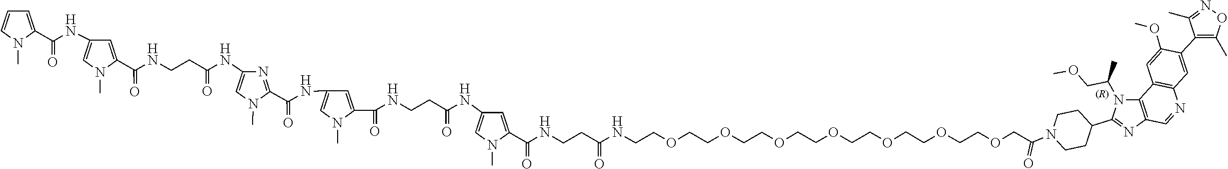 |
| 44 | 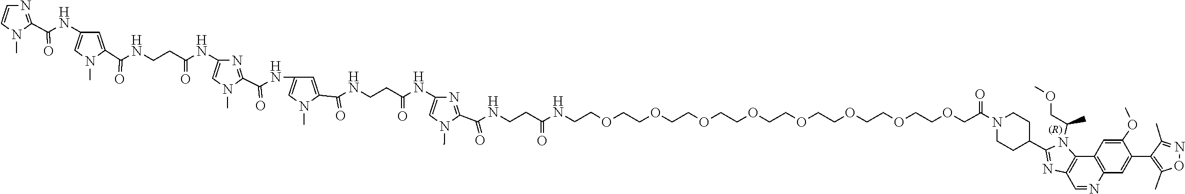 |
| 45 | 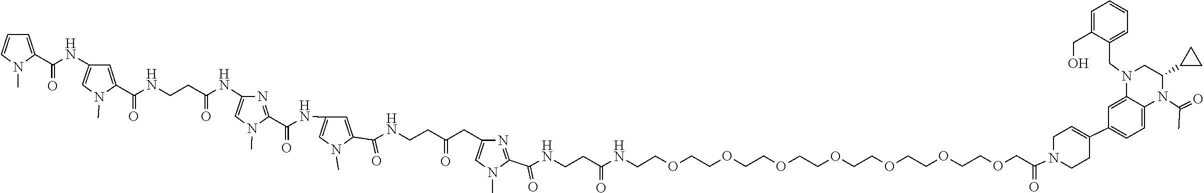 [SMILES: CC(=O)N1c2ccc(C3=CCN(C(=O)COCCOCCOCCOCCOCCOCCOCCNC(=O)CCNC(=O)c4nc(CC(=O)CCNC(=O)c5cc(NC(=O)c6nc(NC(=O)CCNC(=O)c7cc(NC(=O)c8cccn8C)cn7C)cn6C)cn5C)cn4C)CC3)cc2N(Cc2ccccc2CO)C[C@@H]1C1CC1] |
| 46 | 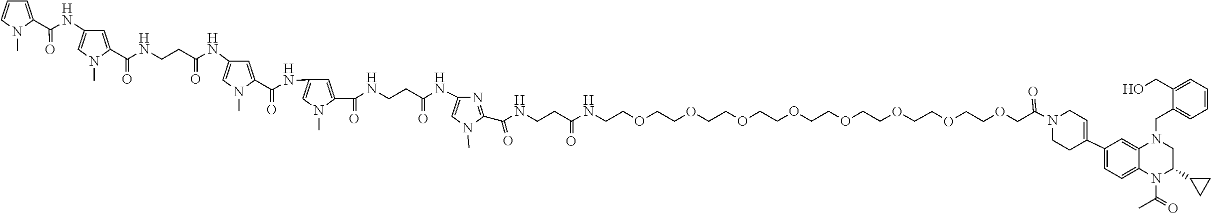 [SMILES: CC(=O)N1c2ccc(C3=CCN(C(=O)COCCOCCOCCOCCOCCOCCOCCOCCNC(=O)CCNC(=O)c4nc(NC(=O)CCNC(=O)c5cc(NC(=O)c6cc(NC(=O)CCNC(=O)c7cc(NC(=O)c8cccn8C)cn7C)cn6C)cn5C)cn4C)CC3)cc2N(Cc2ccccc2CO)C[C@@H]1C1CC1] |
| 47 | 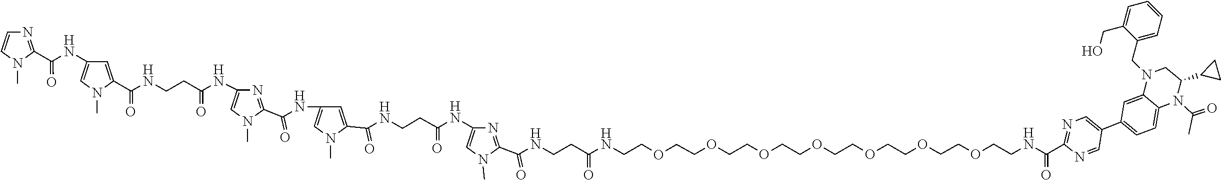 [SMILES: CC(=O)N1c2ccc(-c3cnc(C(=O)NCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)nc3)cc2N(Cc2ccccc2CO)C[C@@H]1C1CC1] |
| 48 | 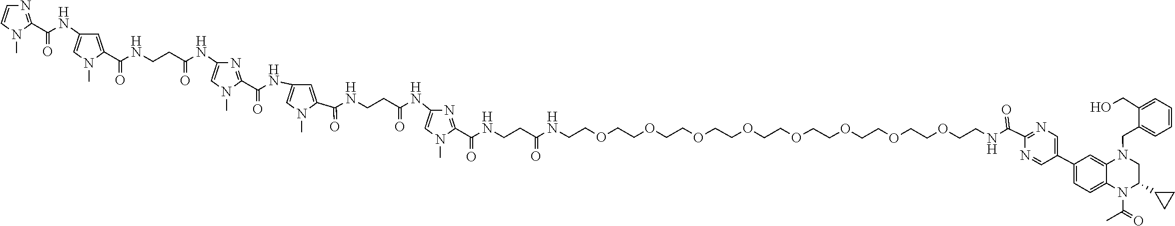 [SMILES: CC(=O)N1c2ccc(-c3cnc(C(=O)NCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)nc3)cc2N(Cc2ccccc2CO)C[C@@H]1C1CC1] |
| 49 | 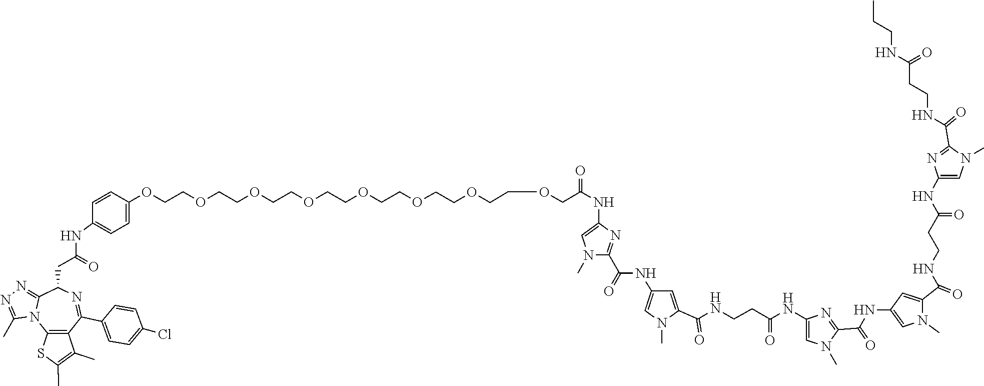 |
| 50 | 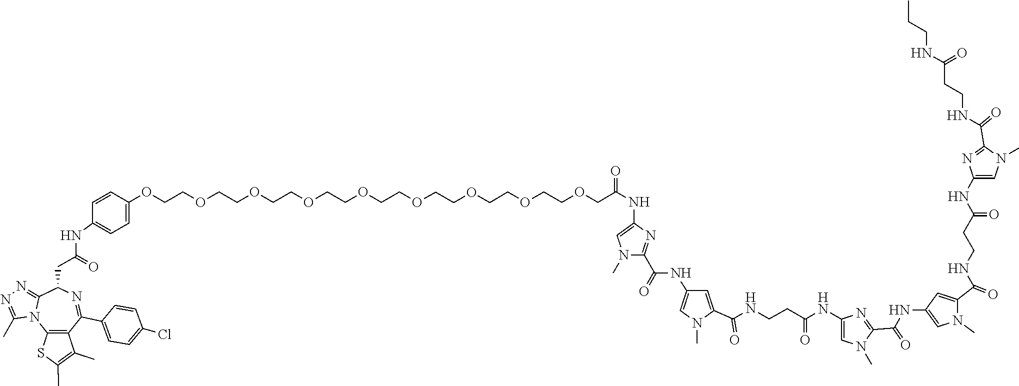 |
| 51 | 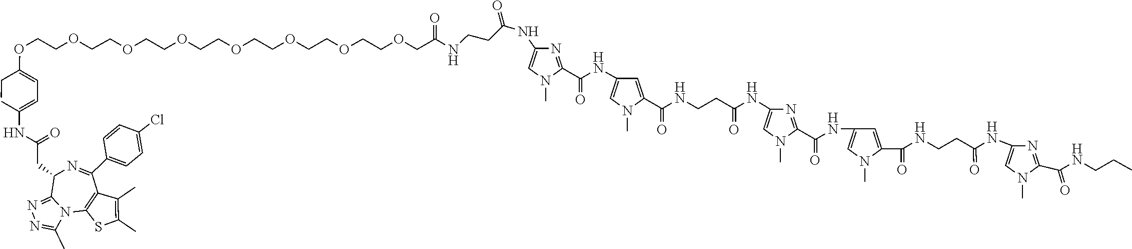 [SMILES: CCCNC(=O)c1nc(NC(=O)CCNC(=O)c2cc(NC(=O)c3nc(NC(=O)CCNC(=O)c4cc(NC(=O)c5nc(NC(=O)CCNC(=O)COCCOCCOCCOCCOCCOCCOCCO/C(=C/C=C(\C)NC(=O)C[C@@H]6N=C(c7ccc(Cl)cc7)c7c(sc(C)c7C)-n7c(C)nnc76)CC)cn5C)cn4C)cn3C)cn2C)cn1C] |
| 52 | 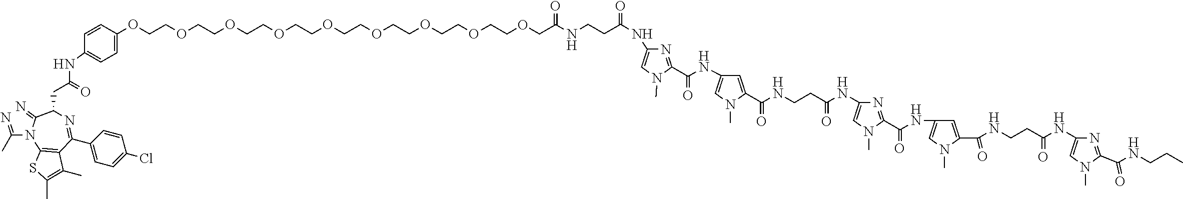 [SMILES: CCCNC(=O)c1nc(NC(=O)CCNC(=O)c2cc(NC(=O)c3nc(NC(=O)CCNC(=O)c4cc(NC(=O)c5nc(NC(=O)CCNC(=O)COCCOCCOCCOCCOCCOCCOCCOCCOc6ccc(NC(=O)C[C@@H]7N=C(c8ccc(Cl)cc8)c8c(sc(C)c8C)-n8c(C)nnc87)cc6)cn5C)cn4C)cn3C)cn2C)cn1C] |
| 53 | 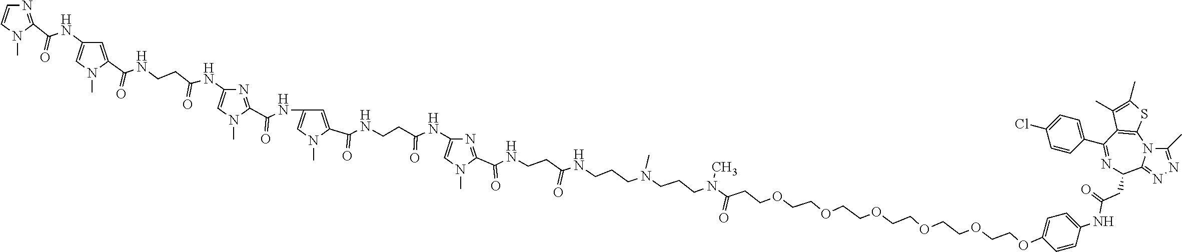 [SMILES: Cc1sc2c(c1C)C(c1ccc(Cl)cc1)=N[C@@H](CC(=O)Nc1ccc(OCCOCCOCCOCCOCCOCCC(=O)N(C)CCCN(C)CCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 54 | 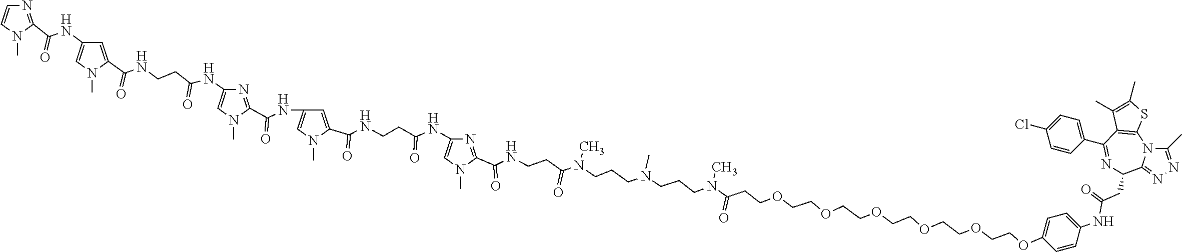 [SMILES: Cc1sc2c(c1C)C(c1ccc(Cl)cc1)=N[C@@H](CC(=O)Nc1ccc(OCCOCCOCCOCCOCCOCCC(=O)N(C)CCCN(C)CCCN(C)C(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 55 | 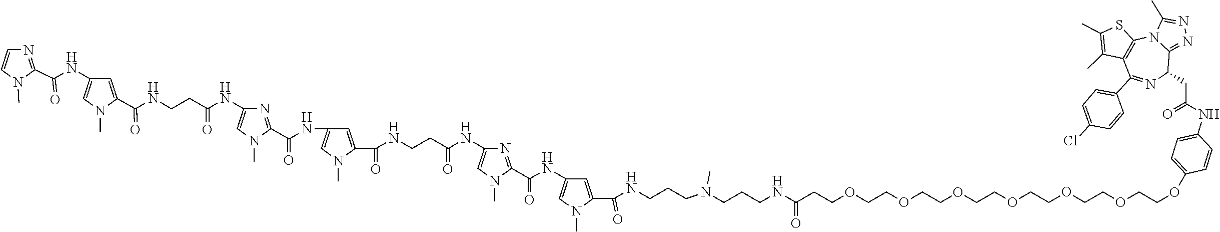 [SMILES: Cc1sc2c(c1C)C(c1ccc(Cl)cc1)=N[C@@H](CC(=O)Nc1ccc(OCCOCCOCCOCCOCCOCCOCCC(=O)NCCCN(C)CCCNC(=O)c3cc(NC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cn3C)cc1)c1nnc(C)n1-2] |
| 56 | 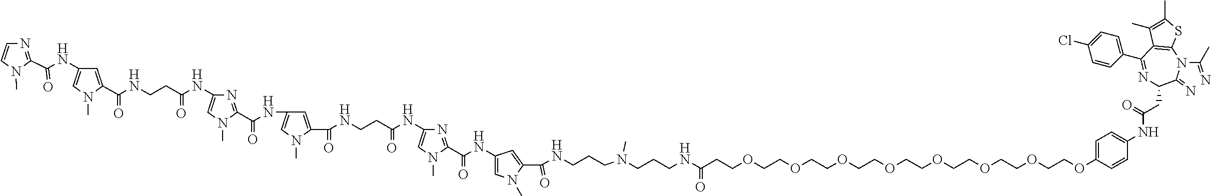 [SMILES: Cc1sc2c(c1C)C(c1ccc(Cl)cc1)=N[C@@H](CC(=O)Nc1ccc(OCCOCCOCCOCCOCCOCCOCCOCCC(=O)NCCCN(C)CCCNC(=O)c3cc(NC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cn3C)cc1)c1nnc(C)n1-2] |
| 57 | 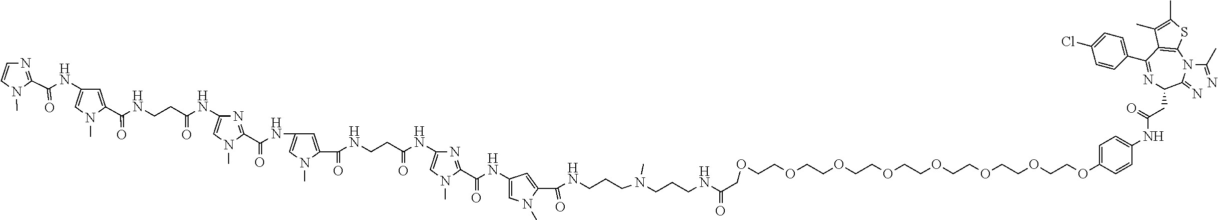 [SMILES: Cc1sc2c(c1C)C(c1ccc(Cl)cc1)=N[C@@H](CC(=O)Nc1ccc(OCCOCCOCCOCCOCCOCCOCCOCC(=O)NCCCN(C)CCCNC(=O)c3cc(NC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cn3C)cc1)c1nnc(C)n1-2] |
| 58 | 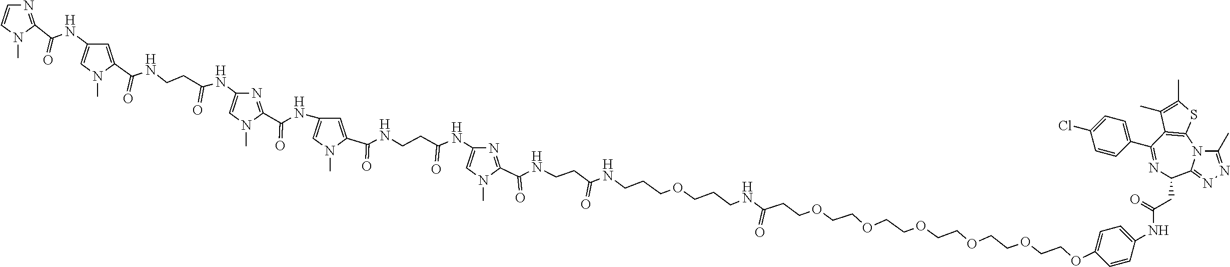 [SMILES: Cc1sc2c(c1C)C(c1ccc(Cl)cc1)=N[C@@H](CC(=O)Nc1ccc(OCCOCCOCCOCCOCCOCCC(=O)NCCCOCCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 59 | 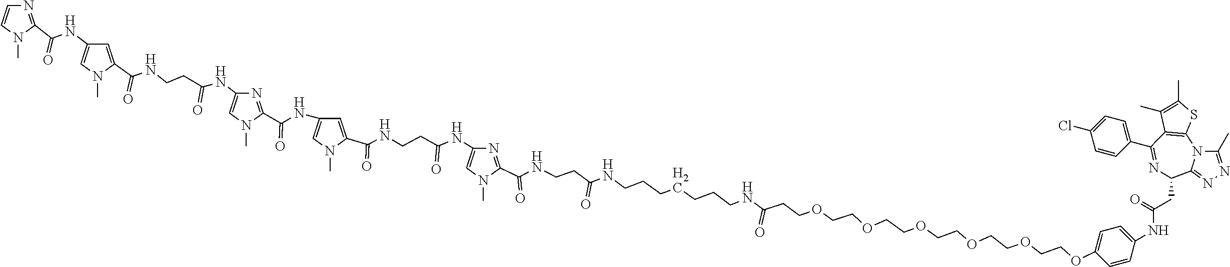 [SMILES: Cc1sc2c(c1C)C(c1ccc(Cl)cc1)=N[C@@H](CC(=O)Nc1ccc(OCCOCCOCCOCCOCCOCCC(=O)NCCCCCCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 60 | 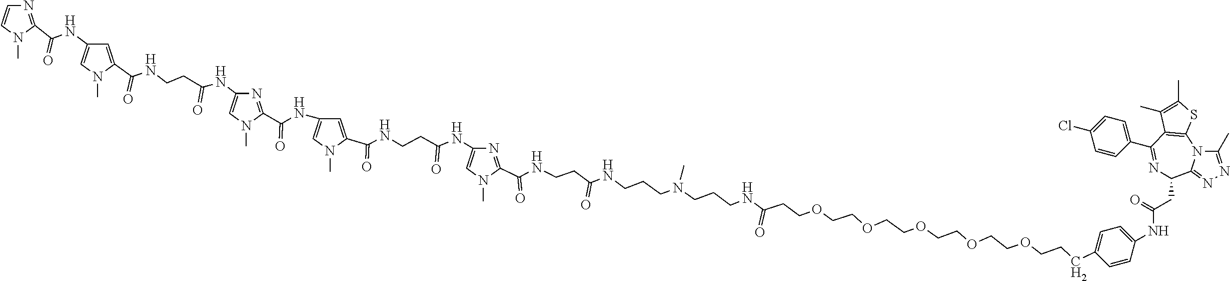 [SMILES: Cc1sc2c(c1C)C(c1ccc(Cl)cc1)=N[C@@H](CC(=O)Nc1ccc(CCCOCCOCCOCCOCCOCCC(=O)NCCCN(C)CCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 61 | 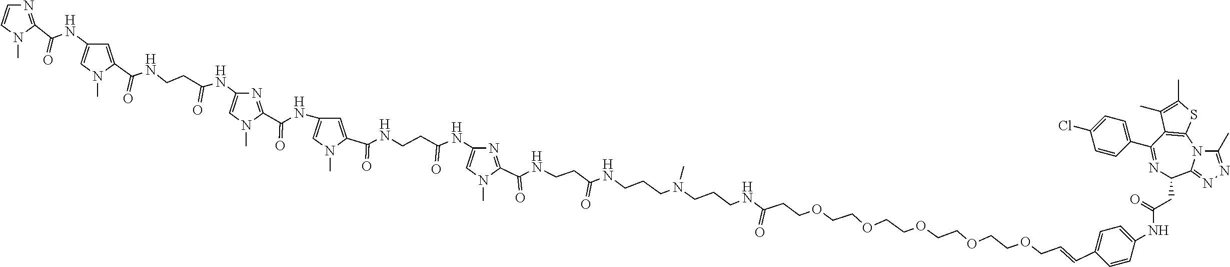 [SMILES: Cc1sc2c(c1C)C(c1ccc(Cl)cc1)=N[C@@H](CC(=O)Nc1ccc(/C=C/COCCOCCOCCOCCOCCC(=O)NCCCN(C)CCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 62 | 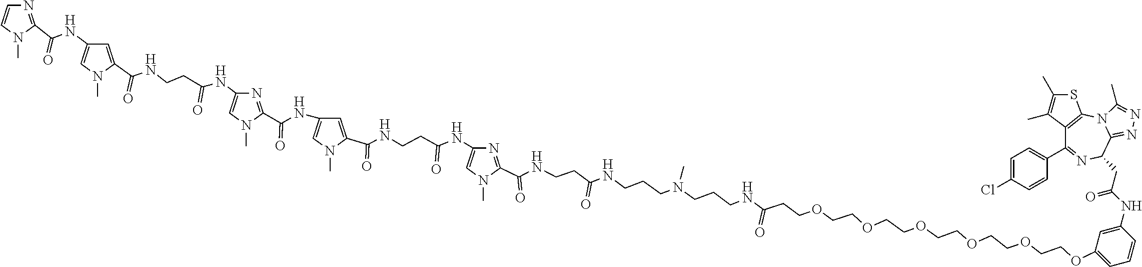 [SMILES: Cc1sc2c(c1C)C(c1ccc(Cl)cc1)=N[C@@H](CC(=O)Nc1cccc(OCCOCCOCCOCCOCCOCCC(=O)NCCCN(C)CCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)c1)c1nnc(C)n1-2] |
| 63 | 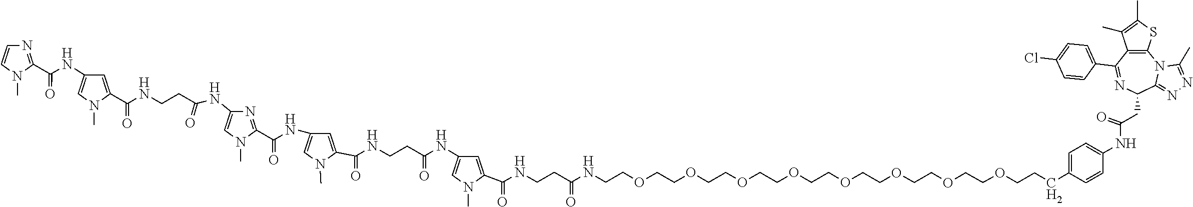 [SMILES: Cc1sc2c(c1C)C(c1ccc(Cl)cc1)=N[C@@H](CC(=O)Nc1ccc(CCCOCCOCCOCCOCCOCCOCCOCCOCCNC(=O)CCNC(=O)c3cc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 64 | 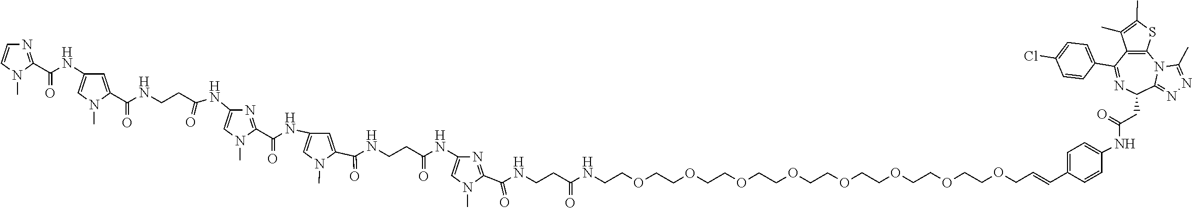 [SMILES: Cc1sc2c(c1C)C(c1ccc(Cl)cc1)=N[C@@H](CC(=O)Nc1ccc(/C=C/COCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] |
| 65 | 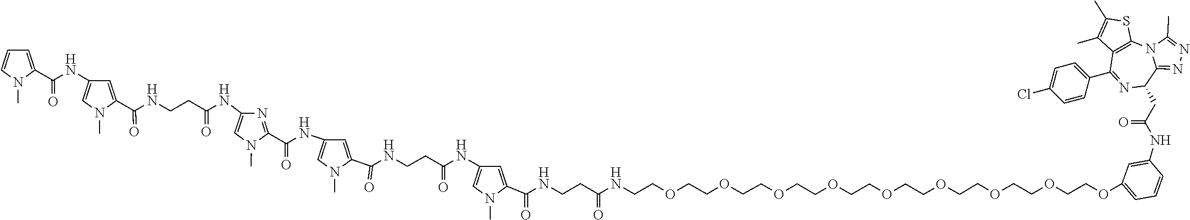 [SMILES: Cc1sc2c(c1C)C(c1ccc(Cl)cc1)=N[C@@H](CC(=O)Nc1cccc(OCCOCCOCCOCCOCCOCCOCCOCCOCCNC(=O)CCNC(=O)c3cc(NC(=O)CCNC(=O)c4cc(NC(=O)c5nc(NC(=O)CCNC(=O)c6cc(NC(=O)c7cccn7C)cn6C)cn5C)cn4C)cn3C)c1)c1nnc(C)n1-2] |
| 66 | 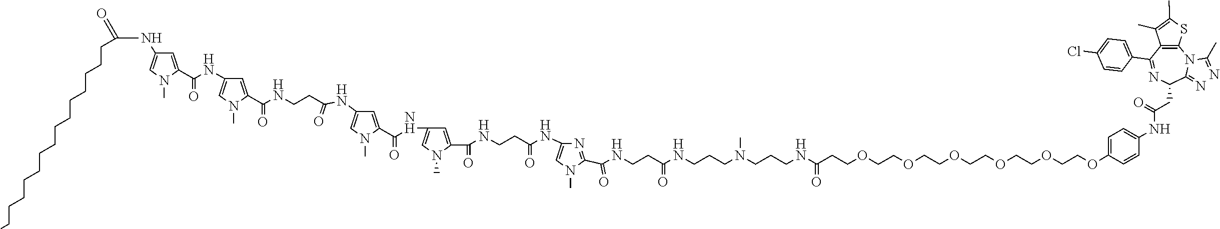 |
| 67 | 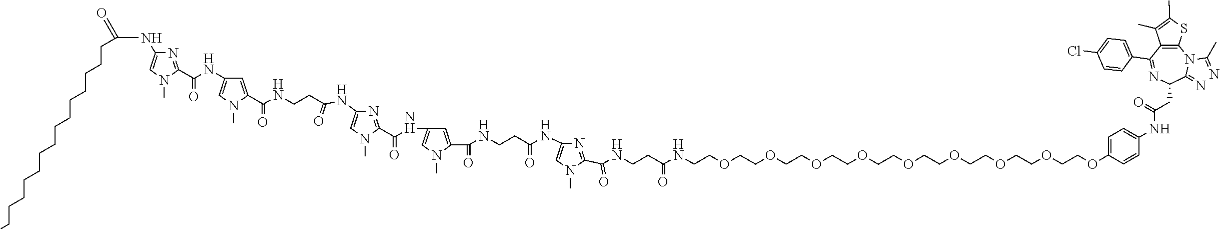 [SMILES: CCCCCCCCCCCCCCCC(=O)Nc1cn(C)c(C(=O)Nc2cc(C(=O)NCCC(=O)Nc3cn(C)c(C(=O)Nc4cc(C(=O)NCCC(=O)Nc5cn(C)c(C(=O)NCCC(=O)NCCOCCOCCOCCOCCOCCOCCOCCOCCOc6ccc(NC(=O)C[C@@H]7N=C(c8ccc(Cl)cc8)c8c(sc(C)c8C)-n8c(C)nnc87)cc6)n5)n(C)c4)n3)n(C)c2)n1] |
| 68 | 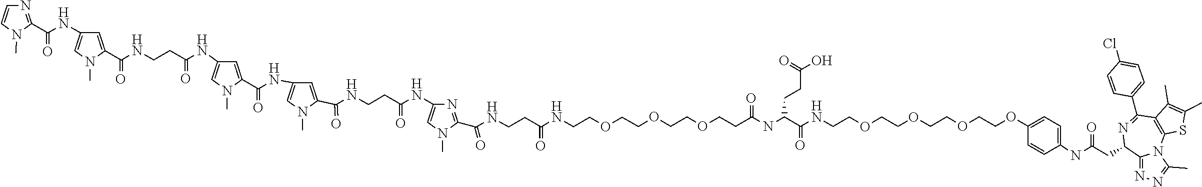 |
| 69 | 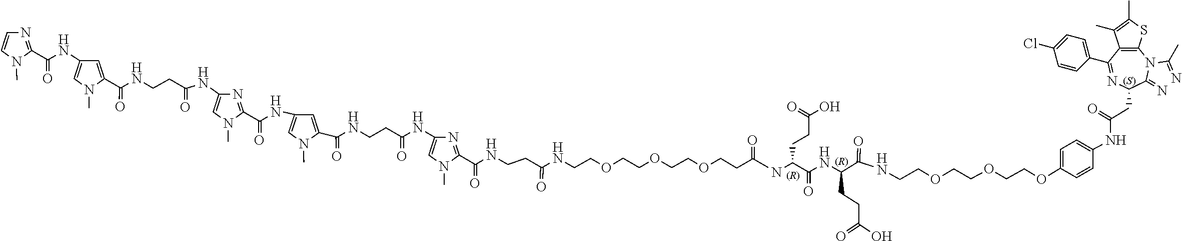 |
| 70 | 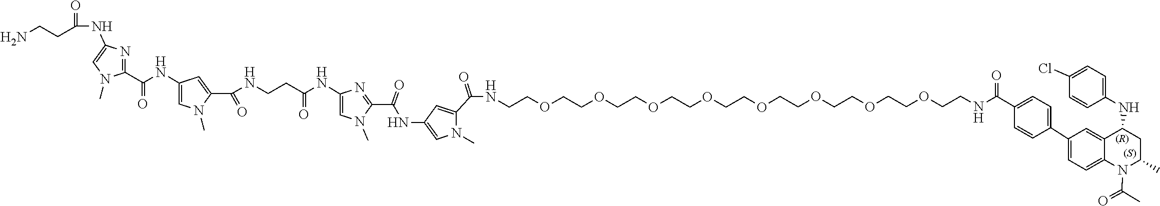 |
| 71 | 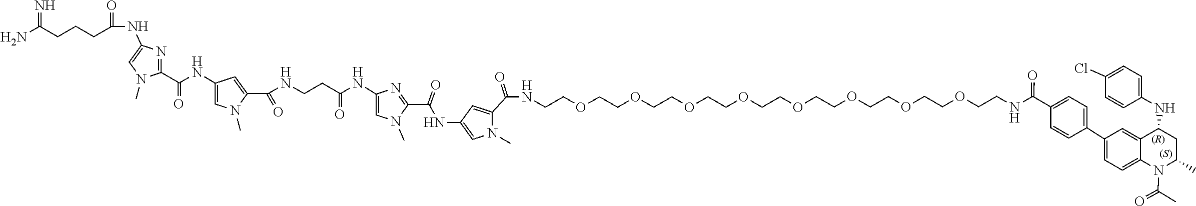 |
| 72 | 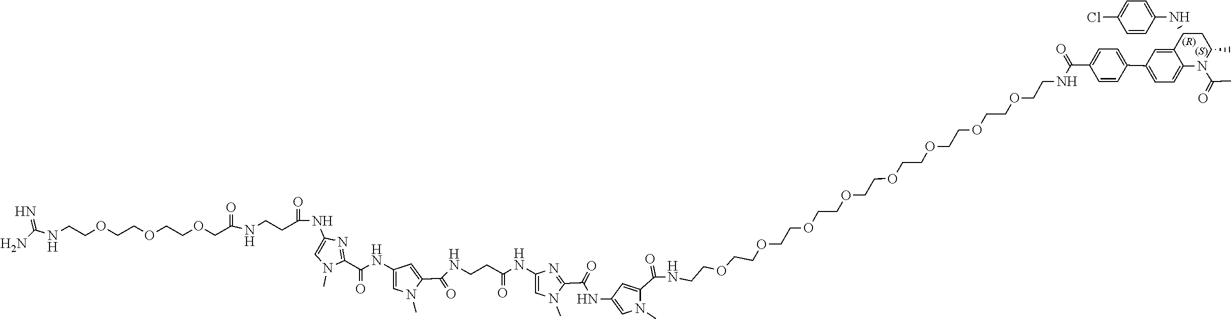 |
| 73 | 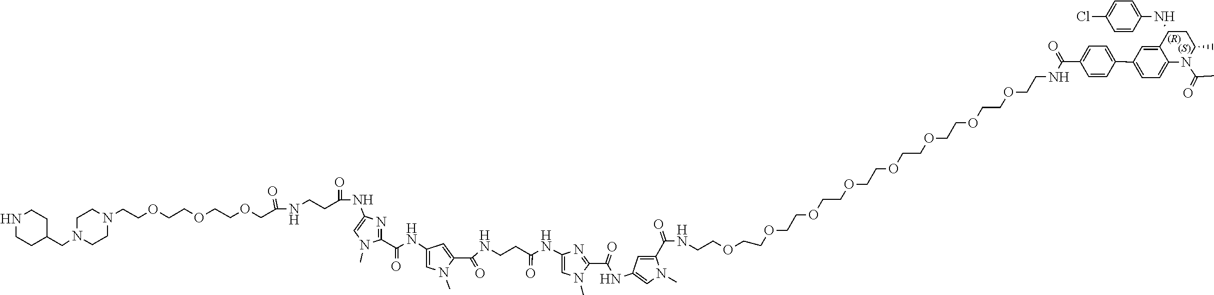 |
| 74 | 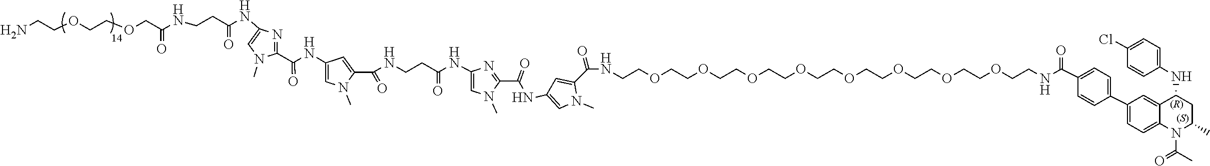 |
| 75 | 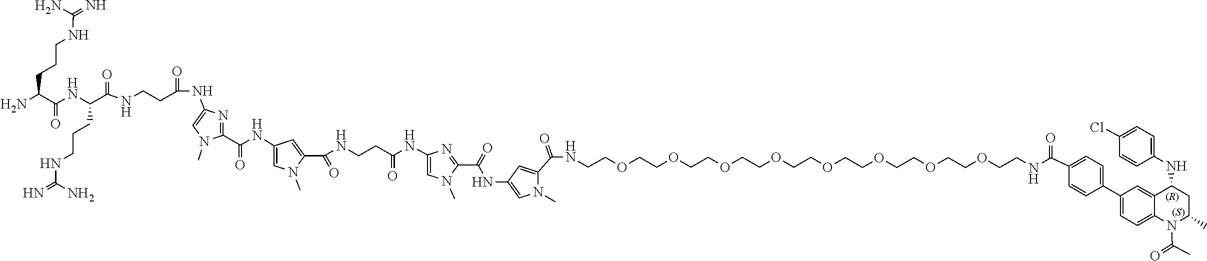 |
| 76 | 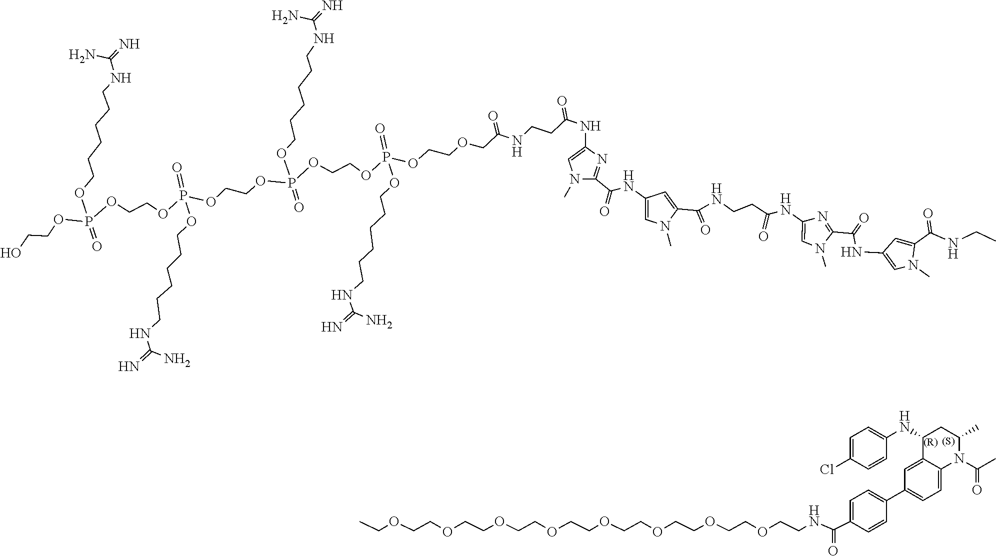 |
| 77 | 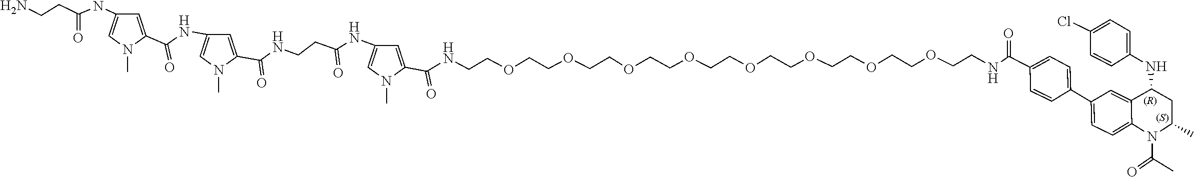 |
| 78 | 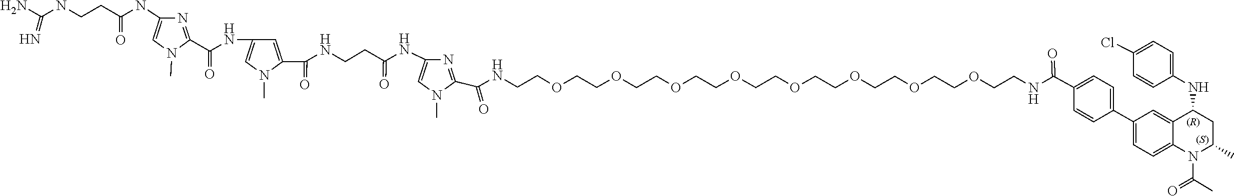 |
| 79 | 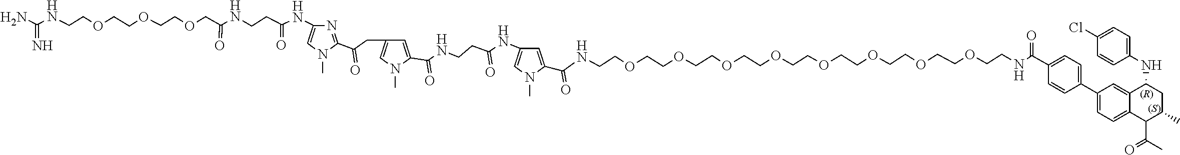 |
| 80 | 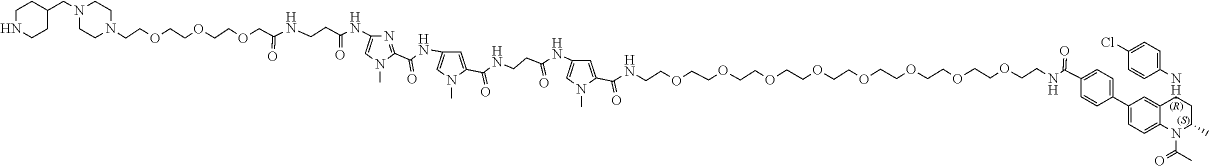 |
| 81 | 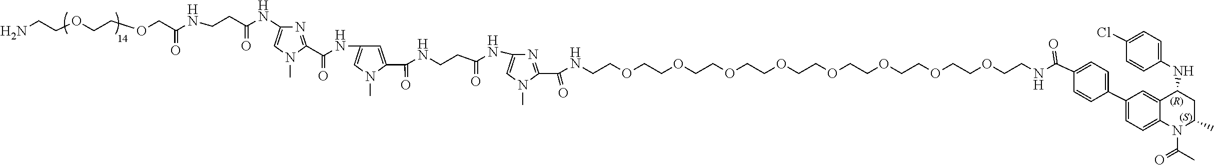 |
| 82 | 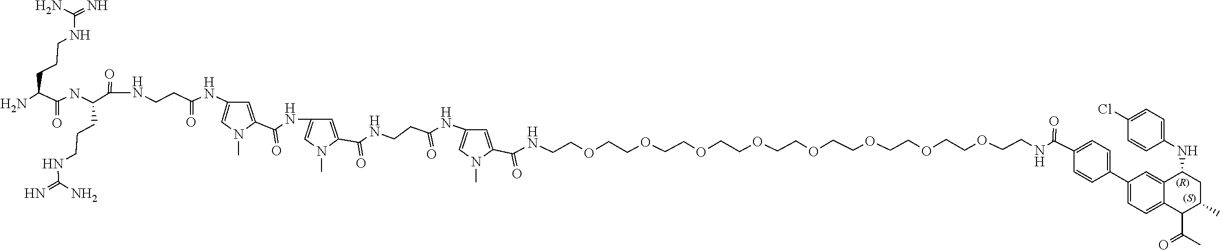 |
| 84 | 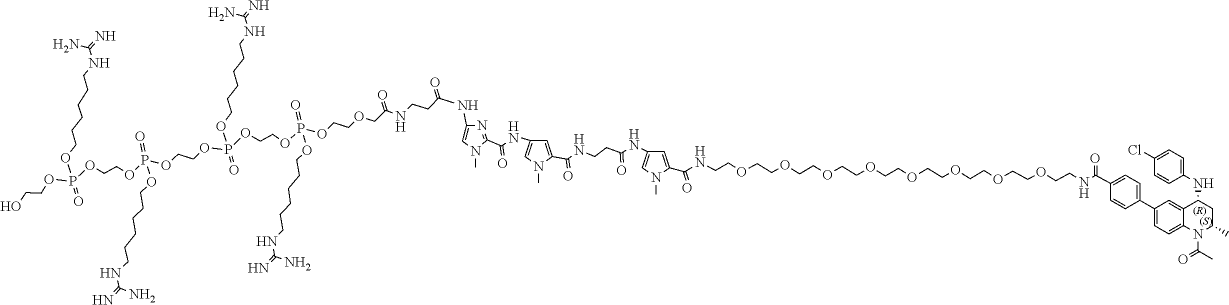 |
| 85 | 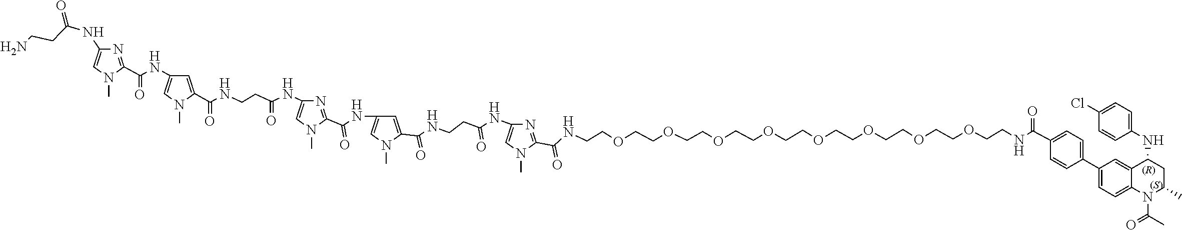 |
| 86 | 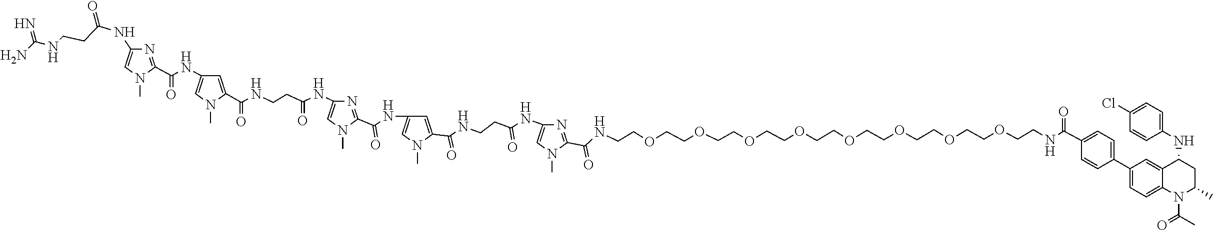 [SMILES: CC(=O)N1c2ccc(-c3ccc(C(=O)NCCOCCOCCOCCOCCOCCOCCOCCOCCNC(=O)c4nc(NC(=O)CCNC(=O)c5cc(NC(=O)c6nc(NC(=O)CCNC(=O)c7cc(NC(=O)c8nc(NC(=O)CCNC(=N)N)cn8C)cn7C)cn6C)cn5C)cn4C)cc3)cc2[C@H](Nc2ccc(Cl)cc2)C[C@@H]1C] |
| 87 | 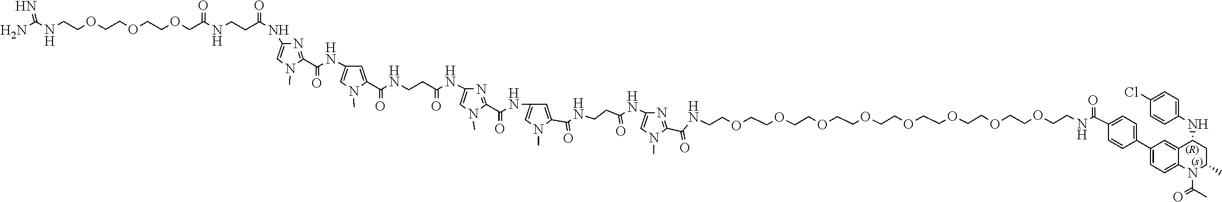 |
| 88 | 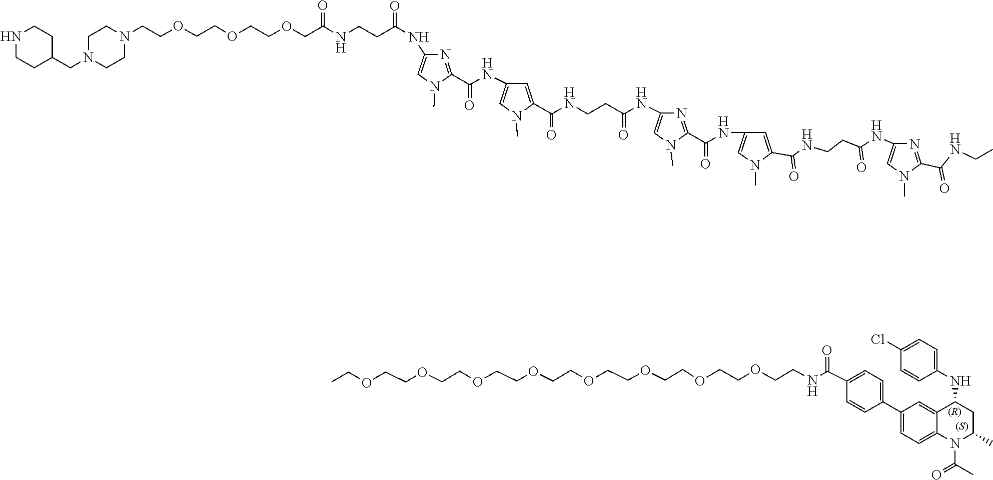 |
| 89 | 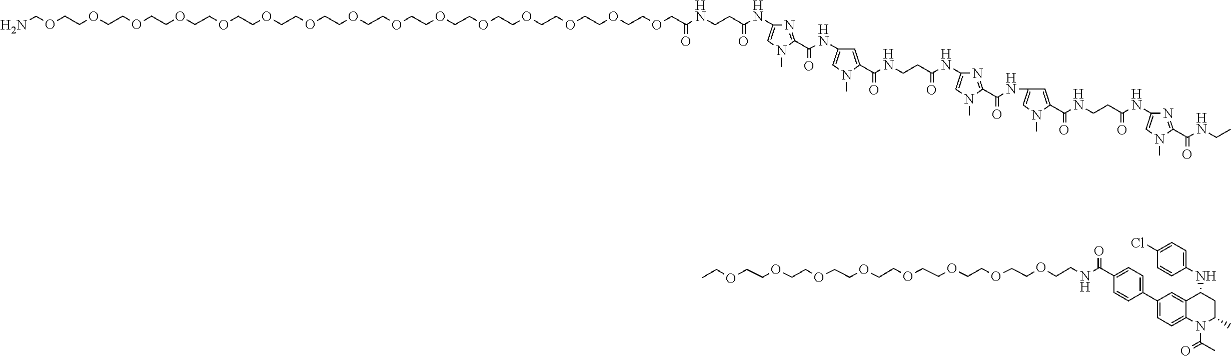 |
| 90 | 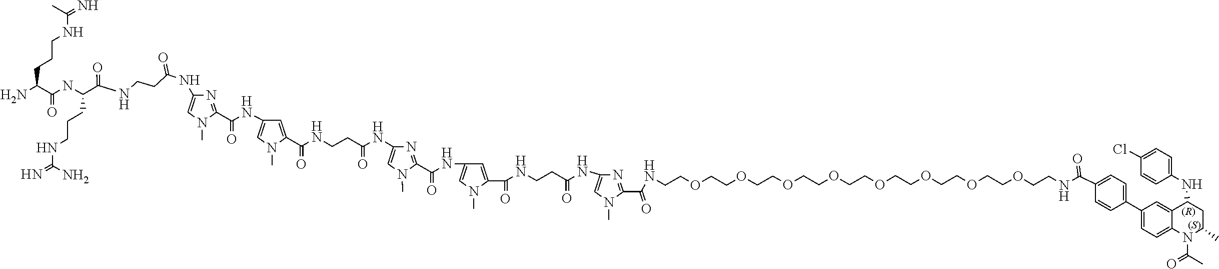 |
| 91 | 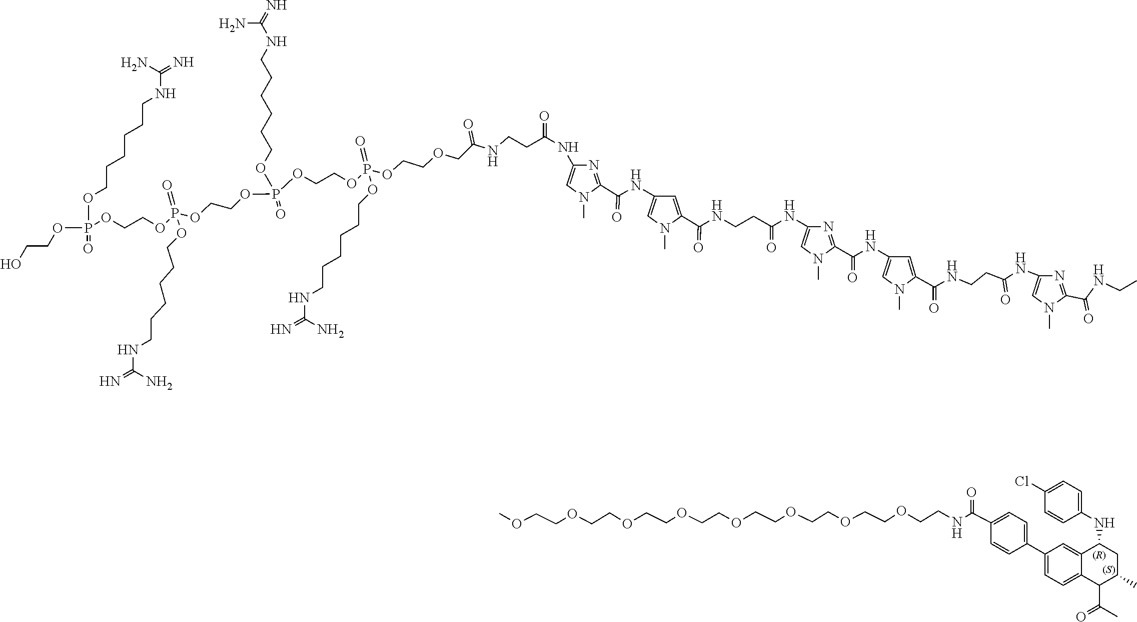 |
| 92 | 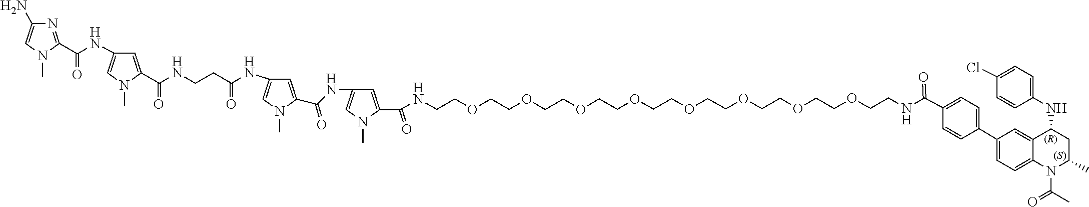 |
| 93 | 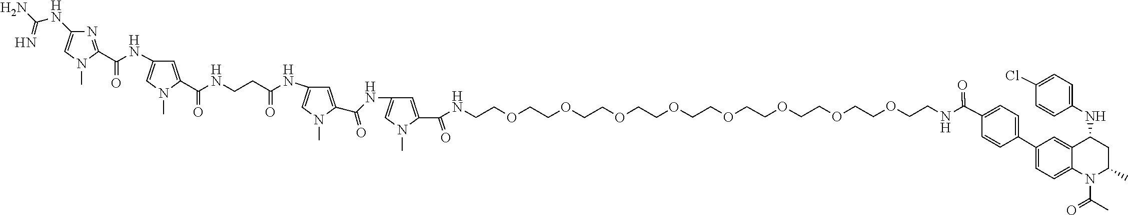 [SMILES: CC(=O)N1c2ccc(-c3ccc(C(=O)NCCOCCOCCOCCOCCOCCOCCOCCOCCNC(=O)c4cc(NC(=O)c5cc(NC(=O)CCNC(=O)c6cc(NC(=O)c7nc(NC(=N)N)cn7C)cn6C)cn5C)cn4C)cc3)cc2[C@H](Nc2ccc(Cl)cc2)C[C@@H]1C] |
| 94 | 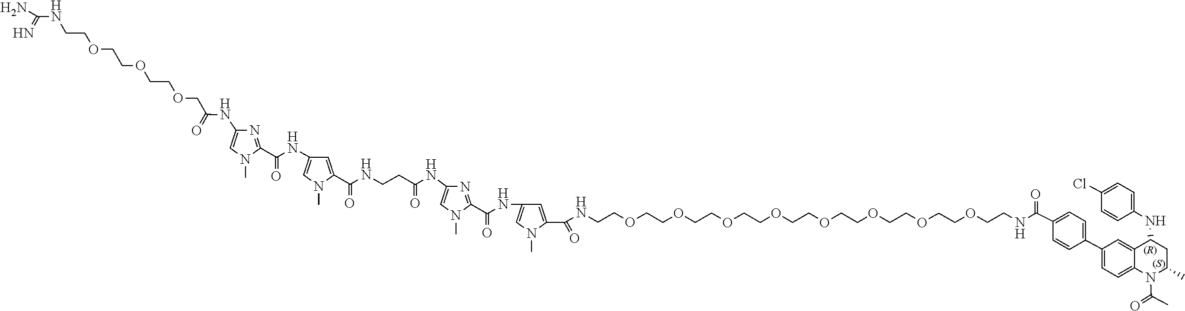 |
| 95 | 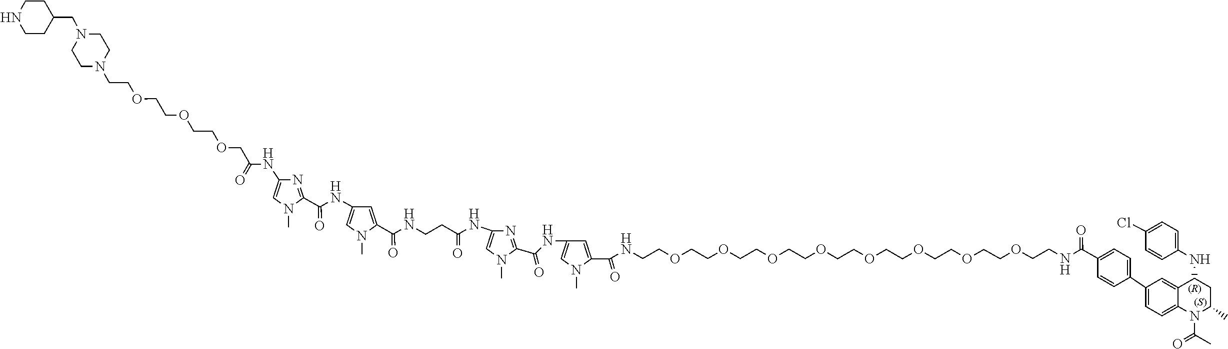 |
| 96 | 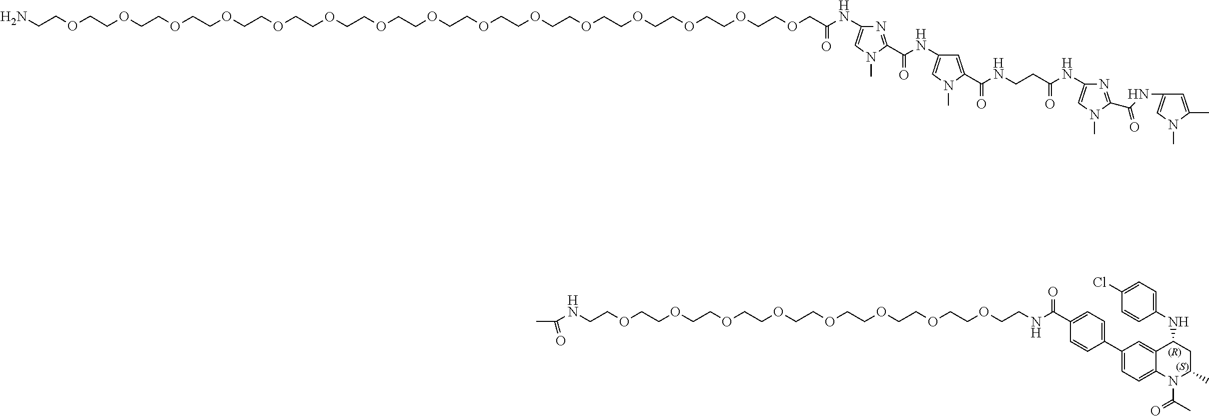 |
| 97 | 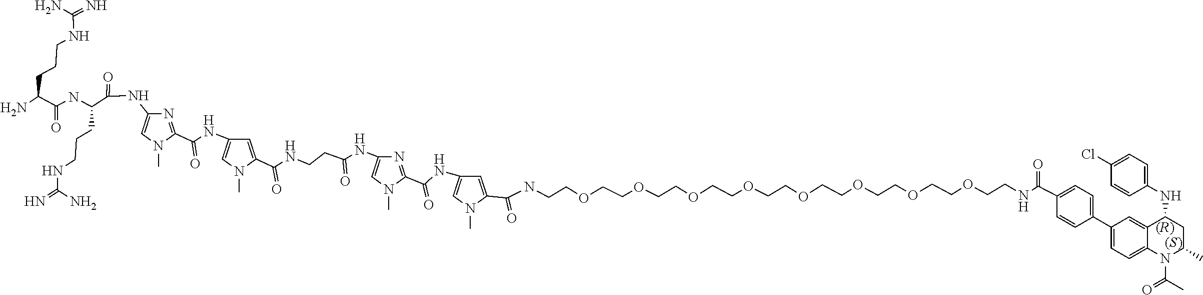 |
| 98 | 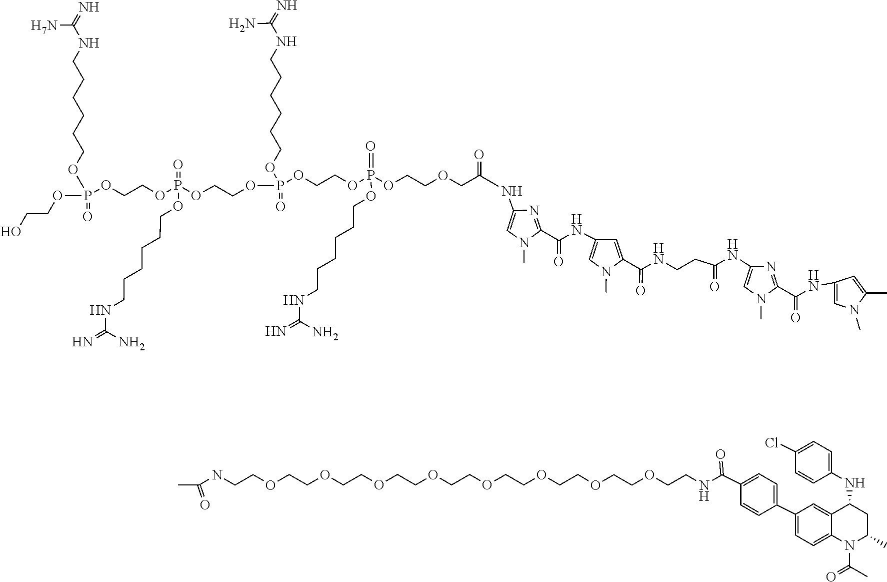 |
| 99 | 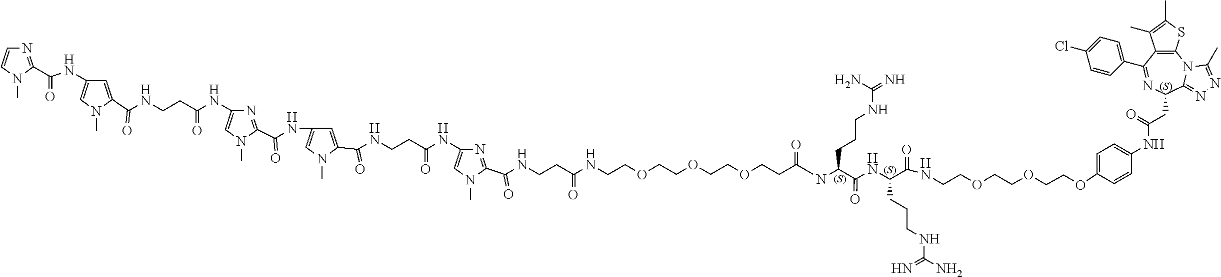 |
| 100 | 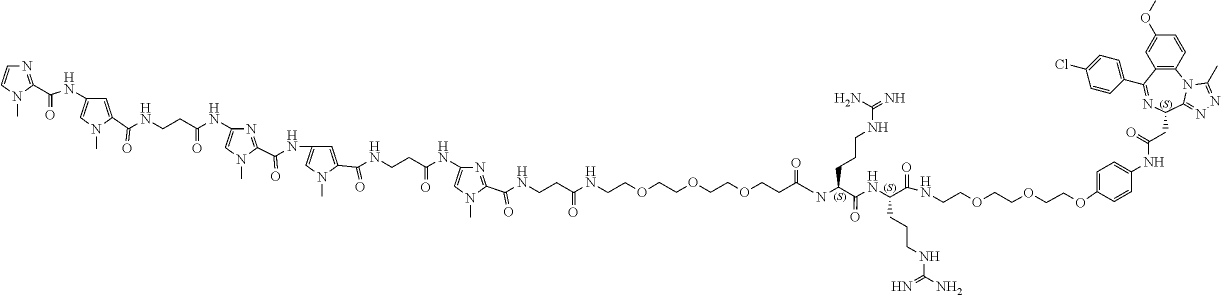 |
| 101 | 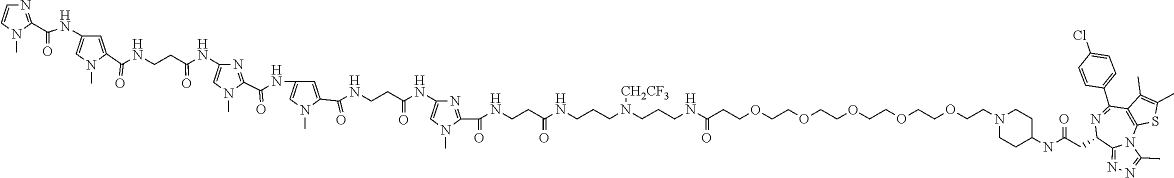 |
| 102 | 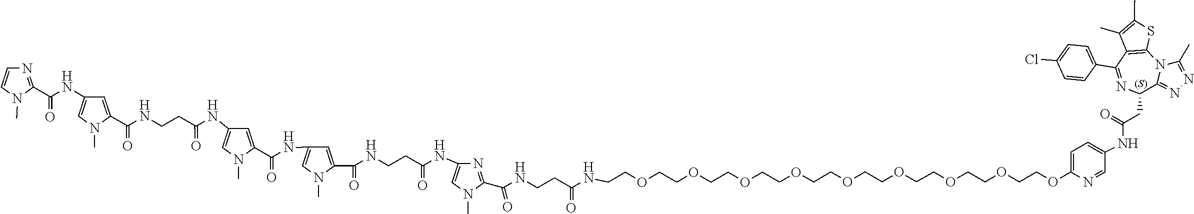 |
| 103 | 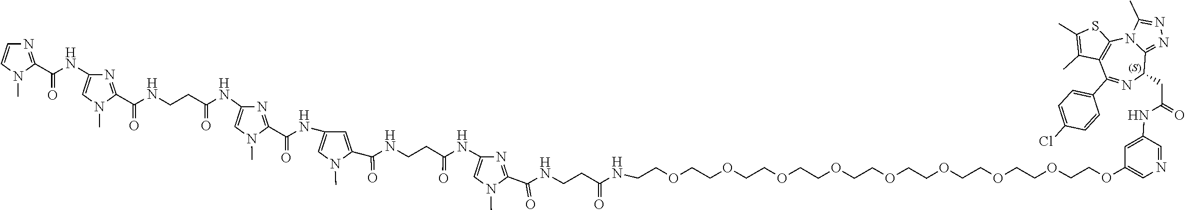 |

### Methods of Use

The present disclosure also relates to a method of modulating the transcription of fxn comprising the step of contacting fxn with a compound as described herein. The cell phenotype, cell proliferation, transcription of fxn, production of mRNA from transcription of fxn, translation of fxn, change in biochemical output produced by the protein coded by fxn, or noncovalent binding of the protein coded by fxn with a natural binding partner may be monitored. Such methods may be modes of treatment of disease, biological assays, cellular assays, biochemical assays, or the like.

Also provided herein is a method of treatment of a disease mediated by transcription of fxn comprising the administration of a therapeutically effective amount of a compound as disclosed herein, or a salt thereof, to a patient in need thereof.

In certain embodiments, the disease is Friedreich's ataxia.

Also provided herein is a compound as disclosed herein for use as a medicament.

Also provided herein is a compound as disclosed herein for use as a medicament for the treatment of a disease mediated by transcription of fxn.

Also provided is the use of a compound as disclosed herein as a medicament.

Also provided is the use of a compound as disclosed herein as a medicament for the treatment of a disease mediated by transcription of fxn.

Also provided is a compound as disclosed herein for use in the manufacture of a medicament for the treatment of a disease mediated by transcription of fxn.

Also provided is the use of a compound as disclosed herein for the treatment of a disease mediated by transcription of fxn.

Also provided herein is a method of modulation of transcription of fxn comprising contacting fxn with a compound as disclosed herein, or a salt thereof.

Also provided herein is a method for achieving an effect in a patient comprising the administration of a therapeutically effective amount of a compound as disclosed herein, or a salt thereof, to a patient, wherein the effect is chosen from improved neural sensation, improved vision, improved balance, improved gait, reduced sensitivity to glucose, and reduced sensitivity to carbohydrates.

Certain compounds of the present disclosure may be effective for treatment of subjects whose genotype has 5 or more repeats of GAA. Certain compounds of the present disclosure may be effective for treatment of subjects whose genotype has 10 or more repeats of GAA. Certain compounds of the present disclosure may be effective for treatment of subjects whose genotype has 20 or more repeats of GAA. Certain compounds of the present disclosure may be effective for treatment of subjects whose genotype has 50 or more repeats of GAA. Certain compounds of the present disclosure may be effective for treatment of subjects whose genotype has 100 or more repeats of GAA. Certain compounds of the present disclosure may be effective for treatment of subjects whose genotype has 200 or more repeats of GAA.

Certain compounds of the present disclosure may be effective for treatment of subjects whose genotype has 500 or more repeats of GAA.

Also provided is a method of modulation of a fxn-mediated function in a subject comprising the administration of a therapeutically effective amount of a compound as disclosed herein.

Also provided is a pharmaceutical composition comprising a compound as disclosed herein, together with a pharmaceutically acceptable carrier.

In certain embodiments, the pharmaceutical composition is formulated for oral administration.

In certain embodiments, the pharmaceutical composition is formulated for intravenous injection and/or infusion.

In certain embodiments, the oral pharmaceutical composition is chosen from a tablet and a capsule.

In certain embodiments, ex vivo methods of treatment are provided. Ex vivo methods typically include cells, organs, and/or tissues removed from the subject. The cells, organs and/or tissues can, for example, be incubated with the agent under appropriate conditions. The contacted cells, organs, and/or tissues are typically returned to the donor, placed in a recipient, or stored for future use. Thus, the compound is generally in a pharmaceutically acceptable carrier.

In certain embodiments, administration of the pharmaceutical composition modulates expression of fxn within 6 hours of treatment. In certain embodiments, administration of the pharmaceutical composition modulates expression of fxn within 24 hours of treatment. In certain embodiments, administration of the pharmaceutical composition modulates expression of fxn within 72 hours of treatment.

In certain embodiments, administration of the pharmaceutical composition causes a 2-fold increase in expression of fxn. In certain embodiments, administration of the pharmaceutical composition causes a 5-fold increase in expression of fxn. In certain embodiments, administration of the pharmaceutical composition causes a 10-fold increase in expression of fxn. In certain embodiments, administration of the pharmaceutical composition causes a 20-fold increase in expression of fxn.

In certain embodiments, administration of the pharmaceutical composition causes a 20% decrease in expression of fxn. In certain embodiments, administration of the pharmaceutical composition causes a 50% decrease in expression of fxn. In certain embodiments, administration of the pharmaceutical composition causes a 80% decrease in expression of fxn. In certain embodiments, administration of the pharmaceutical composition causes a 90% decrease in expression of fxn. In certain embodiments, administration of the pharmaceutical composition causes a 95% decrease in expression of fxn2. In certain embodiments, administration of the pharmaceutical composition causes a 99% decrease in expression of fxn.

In certain embodiments, administration of the pharmaceutical composition causes expression of fxn to fall within 25% of the level of expression observed for healthy individuals. In certain embodiments, administration of the pharmaceutical composition causes expression of fxn to fall within 50% of the level of expression observed for healthy individuals. In certain embodiments, administration of the pharmaceutical composition causes expression of fxn to fall within 75% of the level of expression observed for healthy individuals. In certain embodiments, administration of the pharmaceutical composition causes expression of fxn to fall within 90% of the level of expression observed for healthy individuals.

### Pharmaceutical Compositions and Administration

Also provided is a method of modulation of a fxn-mediated function in a subject comprising the administration of a therapeutically effective amount of a compound as disclosed herein.

Also provided is a pharmaceutical composition comprising a compound as disclosed herein, together with a pharmaceutically acceptable carrier.

In certain embodiments, the pharmaceutical composition is formulated for oral administration.

In certain embodiments, the pharmaceutical composition is formulated for intravenous injection or infusion.

In certain embodiments, the oral pharmaceutical composition is chosen from a tablet and a capsule.

In certain embodiments, ex vivo methods of treatment are provided. Ex vivo methods typically include cells, organs, or tissues removed from the subject. The cells, organs or tissues can, for example, be incubated with the agent under appropriate conditions. The contacted cells, organs, or tissues are typically returned to the donor, placed in a recipient, or stored for future use. Thus, the compound is generally in a pharmaceutically acceptable carrier.

In certain embodiments, the compound is effective at a concentration less than about 5 □M. In certain embodiments, the compound is effective at a concentration less than about 1 □M. In certain embodiments, the compound is effective at a concentration less than about 400 nM. In certain embodiments, the compound is effective at a concentration less than about 200 nM. In certain embodiments, the compound is effective at a concentration less than about 100 nM. In certain embodiments, the compound is effective at a concentration less than about 50 nM. In certain embodiments, the compound is effective at a concentration less than about 20 nM. In certain embodiments, the compound is effective at a concentration less than about 10 nM.

### Abbreviations and Definitions

As used herein, the terms below have the meanings indicated.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene”, “heteroarylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

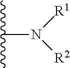

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

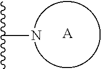

where ring A is a heteroaryl ring containing the depicted nitrogen.

Similarly, when two “adjacent” R groups are said to form a ring “together with the atom to which they are attached,” it is meant that the collective unit of the atoms, intervening bonds, and the two R groups are the recited ring. For example, when the following substructure is present:

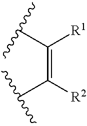

and R¹ and R² are defined as selected from the group consisting of hydrogen and alkyl, or R¹ and R² together with the atoms to which they are attached form an aryl or carbocylyl, it is meant that R¹ and R² can be selected from hydrogen or alkyl, or alternatively, the substructure has structure:

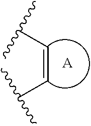

where A is an aryl ring or a carbocylyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

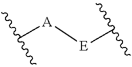

includes the substituent being oriented such that the A is attached at the leftmost attachment point of the molecule as well as the case in which A is attached at the rightmost attachment point of the molecule.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “polyamide” refers to polymers of linkable units chemically bound by amide (i.e., CONH) linkages; optionally, polyamides include chemical probes conjugated therewith. Polyamides may be synthesized by stepwise condensation of carboxylic acids (COOH) with amines (RR′NH) using methods known in the art. Alternatively, polyamides may be formed using enzymatic reactions in vitro, or by employing fermentation with microorganisms.

The term “linkable unit” refers to methylimidazoles, methylpyrroles, and straight and branched chain aliphatic functionalities (e.g., methylene, ethylene, propylene, butylene, and the like) which optionally contain nitrogen Substituents, and chemical derivatives thereof. The aliphatic functionalities of linkable units can be provided, for example, by condensation of B-alanine or dimethylaminopropylamine during synthesis of the polyamide by methods well known in the art.

The term “linker” refers to a chain of at least 10 contiguous atoms. In certain embodiments, the linker contains no more than 20 non-hydrogen atoms. In certain embodiments, the linker contains no more than 40 non-hydrogen atoms. In certain embodiments, the linker contains no more than 60 non-hydrogen atoms. In certain embodiments, the linker contains atoms chosen from C, H, N, O, and S. In certain embodiments, every non-hydrogen atom is chemically bonded either to 2 neighboring atoms in the linker, or one neighboring atom in the linker and a terminus of the linker. In certain embodiments, the linker forms an amide bond with at least one of the two other groups to which it is attached. In certain embodiments, the linker forms an ester or ether bond with at least one of the two other groups to which it is attached. In certain embodiments, the linker forms a thioester or thioether bond with at least one of the two other groups to which it is attached. In certain embodiments, the linker forms a direct carbon-carbon bond with at least one of the two other groups to which it is attached. In certain embodiments, the linker forms an amine or amide bond with at least one of the two other groups to which it is attached. In certain embodiments, the linker comprises —(CH₂OCH₂)— units. In certain embodiments, the linker comprises —(CH(CH₃)OCH₂)— units. In certain embodiments, the linker comprises —(CH₂NR_{N}CH₂) units, for R_{N}=₁₋₄alkyl. In certain embodiments, the linker comprises an arylene, cycloalkylene, or heterocycloalkylene moiety.

The term “spacer” refers to a chain of at least 5 contiguous atoms. In certain embodiments, the spacer contains no more than 10 non-hydrogen atoms. In certain embodiments, the spacer contains atoms chosen from C, H, N, O, and S. In certain embodiments, the spacer forms amide bonds with the two other groups to which it is attached. In certain embodiments, the spacer comprises —(CH₂OCH₂)— units. In certain embodiments, the spacer comprises —(CH₂NR_{N}CH₂)— units, for R_{N}═C₁₋₄alkyl. In certain embodiments, the spacer contains at least one positive charge at physiological pH.

The term “turn component” refers to a chain of about 4 to 10 contiguous atoms. In certain embodiments, the turn component contains atoms chosen from C, H, N, O, and S. In certain embodiments, the turn component forms amide bonds with the two other groups to which it is attached. In certain embodiments, the turn component contains at least one positive charge at physiological pH.

The terms “nucleic acid and “nucleotide” refer to ribonucleotide and deoxyribonucleotide, and analogs thereof, well known in the art.

The term “oligonucleotide sequence” refers to a plurality of nucleic acids having a defined sequence and length (e.g., 2, 3, 4, 5, 6, or even more nucleotides). The term “oligonucleotide repeat sequence” refers to a contiguous expansion of oligonucleotide sequences.

The term “transcription,” well known in the art, refers to the synthesis of RNA (i.e., ribonucleic acid) by DNA-directed RNA polymerase. The term “modulate transcription” refers to a change in transcriptional level which can be measured by methods well known in the art, for example, assay of mRNA, the product of transcription. In certain embodiments, modulation is an increase in transcription. In other embodiments, modulation is a decrease in transcription

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa.

The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amide,” as used herein, alone in combination, refers to —C(O)NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted. Amides may be formed by direct condensation of carboxylic acids with amines, or by using acid chlorides. In addition, coupling reagents are known in the art, including carbodiimide-based compounds such as DCC and EDCI.

The term “amino,” as used herein, alone or in combination, refers to —NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl. The term “arylene” embraces aromatic groups such as phenylene, naphthylene, anthracenylene, and phenanthrylene.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group- with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or combinations thereof, fully saturated or containing from 1 to 3 degrees of unsaturation, consisting of the stated number of carbon atoms and from one to three heteroatoms chosen from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently chosen from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include tetrahydroisoquinoline, aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds or molecules of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms (i.e., C₁-C₆ alkyl).

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four said members may be heteroatoms chosen from N, O, and S, or 2) bicyclic heteroaryl, wherein each of the fused rings comprises five or six ring members, comprising between them one to four heteroatoms chosen from N, O, and S.

The term “lower cycloalkyl,” as used herein, alone or in combination, means a monocyclic cycloalkyl having between three and six ring members (i.e., C₃-C₆ cycloalkyl). Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms chosen from N, O, and S (i.e., C₃-C₆ heterocycloalkyl). Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently chosen from hydrogen and lower alkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with”.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁—C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and R″ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

Asymmetric centers exist in the compounds or molecules disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds or molecules can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds or molecules of particular stereochemistry are either commercially available or can be made and resolved by techniques known in the art. Additionally, the compounds or molecules disclosed herein may exist as geometric isomers. The present disclosure includes all cis, trans, syn, anti, entgegen (E), and zusammen (Z) isomers as well as the appropriate mixtures thereof Additionally, compounds or molecules may exist as tautomers; all tautomeric isomers are provided by this disclosure. Additionally, the compounds or molecules disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder or on the effecting of a clinical endpoint.

The term “therapeutically acceptable” refers to those compounds or molecules (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “prodrug” refers to a compound or molecule that is made more active in vivo. Certain compounds or molecules disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds or molecules disclosed herein can exist as therapeutically acceptable salts. The present disclosure includes compounds or molecules listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound or molecule in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

Basic addition salts can be prepared during the final isolation and purification of the compounds or molecules by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

Other carrier materials and modes of administration known in the pharmaceutical art may also be used. Pharmaceutical compositions of the disclosure may be prepared by any of the well-known techniques of pharmacy, such as effective formulation and administration procedures. Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

The amount of active ingredient that may be combined with the carrier materials to produce a single dosage form will vary depending upon the host treated and the particular mode of administration.

The compounds or molecules can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. In addition, the route of administration may vary depending on the condition and its severity. The above considerations concerning effective formulations and administration procedures are well known in the art and are described in standard textbooks.

### Combinations and Combination Therapy

In certain instances, it may be appropriate to administer at least one of the compounds described herein (or a pharmaceutically acceptable salt thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds of the disclosure with an ACE inhibitor.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating fxn-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of fxn-mediated disorders.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Compound Synthesis

Compounds of the present disclosure can be prepared using methods illustrated in general synthetic schemes and experimental procedures detailed below. General synthetic schemes and experimental procedures are presented for purposes of illustration and are not intended to be limiting. Starting materials used to prepare compounds of the present disclosure are commercially available or can be prepared using routine methods known in the art.

### List of Abbreviation

Ac₂O=acetic anhydride; AcCl=acetyl chloride; AcOH=acetic acid; AIBN=azobisisobutyronitrile; aq.=aqueous; Bu₃SnH=tributyltin hydride; CD₃OD=deuterated methanol; CDCl₃=deuterated chloroform; CDI=1,1′-Carbonyldiimidazole; DBU=1,8-diazabicyclo[5.4.0]undec-7-ene; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethylformamide; DMSO-d₆=deuterated dimethyl sulfoxide; DMSO=dimethyl sulfoxide; DPPA=diphenylphosphoryl azide; EDC.HCl=EDCI.HCl=1-ethyl-3-(3-dimethylaminopropyl)carbodiimide hydrochloride; Et₂O=diethyl ether; EtOAc=ethyl acetate; EtOH=ethanol; h=hour; HATU=2-(1H-7-azabenzotriazol-1-yl)-1,1,3,3-tetramethyl uronium hexafluorophosphate methanaminium; HMDS=hexamethyldisilazane; HOBT=1-hydroxybenzotriazole; i-PrOH=isopropanol; LAH=lithium aluminium hydride; LiHMDS=Lithium bis(trimethylsilyl)amide; MeCN=acetonitrile; MeOH=methanol; MP-carbonate resin=macroporous triethylammonium methylpolystyrene carbonate resin; MsCl=mesyl chloride; MTBE=methyl tertiary butyl ether; MW=microwave irradiation; n-BuLi=n-butyllithium; NaHMDS=Sodium bis(trimethylsilyl)amide; NaOMe=sodium methoxide; NaOtBu=sodium t-butoxide; NBS=N-bromosuccinimide; NCS=N-chlorosuccinimide; NMP=N-Methyl-2-pyrrolidone; Pd(Ph₃)₄=tetrakis(triphenylphosphine)palladium(0); Pd₂(dba)₃=tris(dibenzylideneacetone)-dipalladium(0); PdCl₂(PPh₃)₂=bis(triphenylphosphine)palladium(II) dichloride; PG=protecting group; prep-HPLC=preparative high-performance liquid chromatography; PyBop=(benzotriazol-1-yloxy)-tripyrrolidinophosphonium hexafluorophosphate; Pyr=pyridine; RT=room temperature; RuPhos=2-dicyclohexylphosphino-2′,6′-diisopropoxybiphenyl; sat.=saturated; ss=saturated solution; t-BuOH=tert-butanol; T3P=Propylphosphonic Anhydride; TBS=TBDMS=tert-butyldimethylsilyl; TBSCl=TBDMSCl=tert-butyldimethylchlorosilane; TEA=Et₃N=triethylamine; TFA=trifluoroacetic acid; TFAA=trifluoroacetic anhydride; THF=tetrahydrofuran; Tol=toluene; TsCl=tosyl chloride; XPhos=2-dicyclohexylphosphino-2′,4′,6′-triisopropylbiphenyl.

### General Synthetic Methods for Preparing Compounds

In general, polyamides of the present disclosure may be synthesized by solid supported synthetic methods, using compounds such as Boc-protected straight chain aliphatic and heteroaromatic amino acids, and alkylated derivatives thereof, which are cleaved from the support by aminolysis, deprotected (e.g., with sodium thiophenoxide), and purified by reverse-phase HPLC, as well known in the art. The identity and purity of the polyamides may be verified using any of a variety of analytical techniques available to one skilled in the art such as ¹H-NMR, analytical HPLC, or mass spectrometry.

The following scheme can be used to practice the present disclosure.

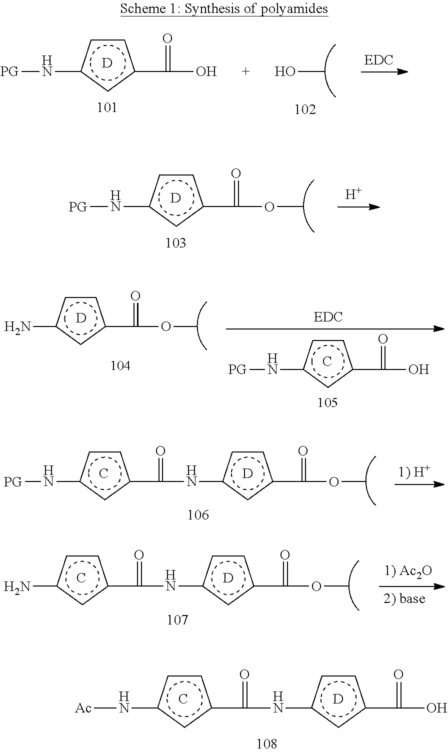

The compounds disclosed herein can be synthesized using Scheme I. For clarity and compactness, the scheme depicts the synthesis of a diamide comprising subunits “C” and “D”, both of which are represented as unspecified five-membered rings having amino and carboxy moieties. The amino group of subunit “D” is protected with a protecting group “PG” such as a Boc or CBz carbamate to give 101. The free)carboxylic acid is then reacted with a solid support, using a coupling reagent such as EDC, to give the supported compound 103. Removal of PG under acidic conditions gives the free amine 104, which is coupled with the nitrogen-protected carboxylic acid 105 to give amide 106. Removal of PG under acidic conditions gives the free amine 107. In this example, the free amine is reacted with acetic anhydride to form an acetamide (not shown. The molecule is then cleaved from the solid support under basic conditions to give carboxylic acid 108. Methods for attachment of the linker L and recruiting moiety X are disclosed below.

The person of skill will appreciate that many variations of the above scheme are available to provide a wide range of compounds:
1) The sequence 104-106-107 can be repeated as often as desired, in order to form longer polyamine sequences.
2) A variety of amino heterocycle carboxylic acids can be used, to form different subunits. Table 4, while not intended to be limiting, provides several heterocycle amino acids that are contemplated for the synthesis of the compounds in this disclosure. Carbamate protecting groups PG can be incorporated using techniques that are well established in the art.

TABLE 4
Heterocyclic amino acids.
Structure

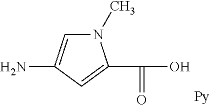

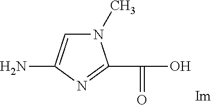

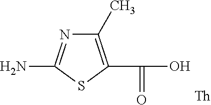

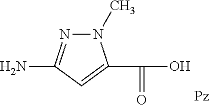

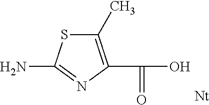

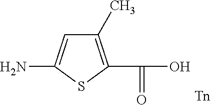

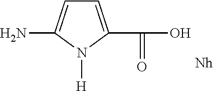

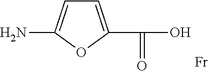

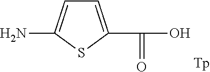

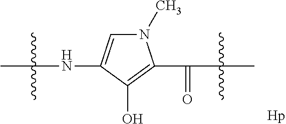 [SMILES: *Nc1cn(C)c(C(*)=O)c1O ; "Hp"]

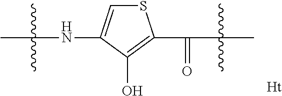 [SMILES: *Nc1csc(C(*)=O)c1O ; "Ht"]

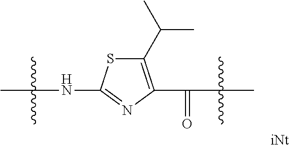 [SMILES: *Nc1nc(C(*)=O)c(C(C)C)s1 ; "iNt"]

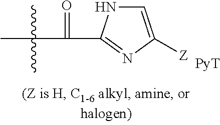

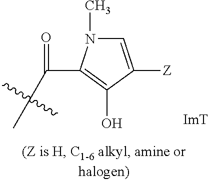

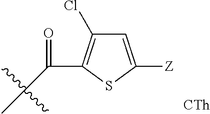

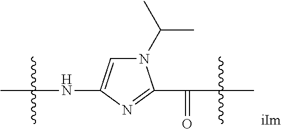 [SMILES: *Nc1cn(C(C)C)c(C(*)=O)n1 ; "iIm"]

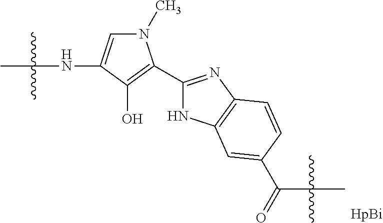 [SMILES: *Nc1cn(C)c(-c2nc3ccc(C(*)=O)cc3[nH]2)c1O ; "HpBi"]

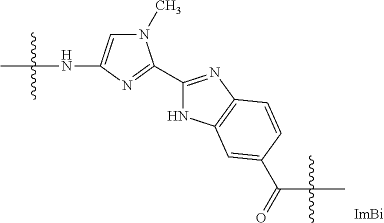 [SMILES: *Nc1cn(C)c(-c2nc3ccc(C(*)=O)cc3[nH]2)n1 ; "ImBi"]

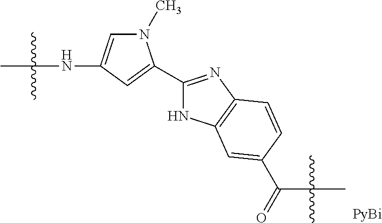 [SMILES: *Nc1cc(-c2nc3ccc(C(*)=O)cc3[nH]2)n(C)c1 ; "PyBi"]

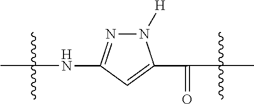 [SMILES: *Nc1cc(C(*)=O)[nH]n1]

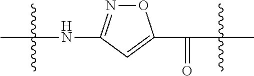 [SMILES: *Nc1cc(C(*)=O)on1]

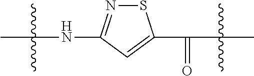 [SMILES: *Nc1cc(C(*)=O)sn1]

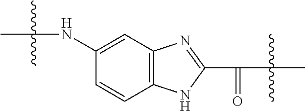 [SMILES: *Nc1ccc2[nH]c(C(*)=O)nc2c1]

3) Hydroxy-containing heterocyclic amino acids can be incorporated into Scheme I as their TBS ethers. While not intended to be limiting, Scheme II provides the synthesis of TBS-protected heterocyclic amino acids contemplated for the synthesis of the molecules in this disclosure.

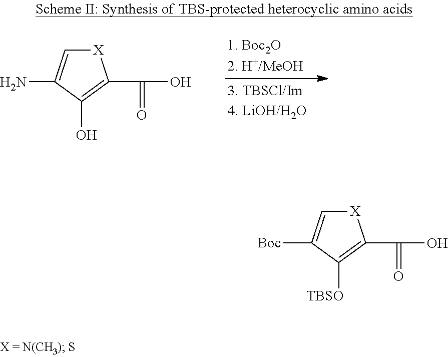

4) Aliphatic amino acids can be used in the above synthesis for the formation of spacer units “W” and subunits for recognition of DNA nucleotides. Table 5, while not intended to be limiting, provides several aliphatic amino acids contemplated for the synthesis of the or molecules in this disclosure.

TABLE 5
Aliphatic amino acids
Structure

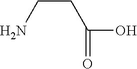

beta-alanine (□)

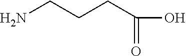

gamma-aminobutyric acid (“gAB” or □)

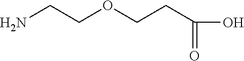

3-(2-aminoethoxy)propanoic acid

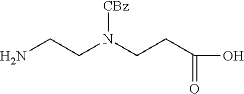

3-((2-aminoethyl)(2-oxo-2-phenyl-1β²-ethyl)amino)propanoic acid

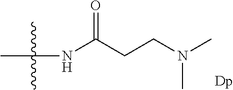 [SMILES: *NC(=O)CCN(C)C]

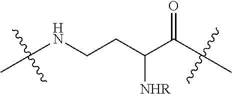

(R is H, C₁₋₆ alkyl)

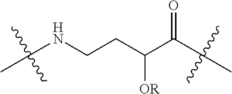

(R is H, C₁₋₆ alkyl, aryl, or heteroaryl)

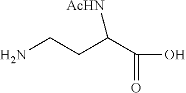 [SMILES: CC(=O)NC(CCN)C(=O)O]

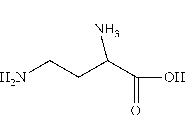

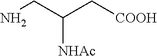 [SMILES: CC(=O)NC(CN)CC(=O)O]

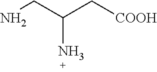

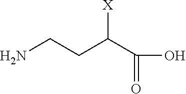

X is F or OH

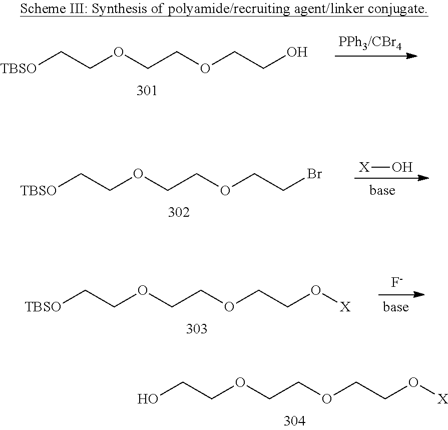

Attachment of the linker L and recruiting moiety X can be accomplished with the methods disclosed in Scheme III, which uses a triethylene glycol moiety for the linker L. The mono-TBS ether of triethylene glycol 301 is converted to the bromo compound 302 under Mitsunobu conditions. The recruiting moiety X is attached by displacement of the bromine with a hydroxyl moiety, affording ether 303. The TBS group is then removed by treatment with fluoride, to provide alcohol 304, which will be suitable for coupling with the polyamide moiety. Other methods will be apparent to the person of skill in the art for inclusion of alternate linkers L, including but not limited to propylene glycol or polyamine linkers, or alternate points of attachment of the recruiting moiety X, including but not limited to the use of amines and thiols.

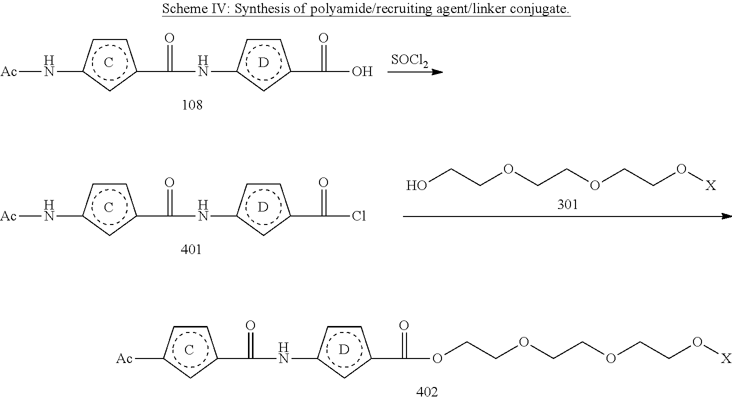

Synthesis of the X-L-Y molecule can be completed with the methods set forth in Scheme IV. Carboxylic acid 108 is converted to the acid chloride 401. Reaction with the alcohol functionality of 301 under basic conditions provides the coupled product 402. Other methods will be apparent to the person of skill in the art for performing the coupling procedure, including but not limited to the use of carbodiimide reagents. For instance, the amide coupling reagents can be used, but not limited to, are carbodiimides such as dicyclohexylcarbodiimide (DCC), diisopropylcarbodiimide (DIC), ethyl-(N′,N′-dimethylamino)propylcarbodiimide hydrochloride (EDC), in combination with reagents such as 1-hydroxybenzotriazole (HOBt), 4-(N,N-dimethylamino)pyridine (DMAP) and diisopropylethylamine (DIEA). Other reagents are also often used depending the actual coupling reactions are (Benzotriazol-1-yloxy)tris(dimethylamino)phosphonium hexafluorophosphate (BOP), (Benzotriazol-1-yloxy)tripyrrolidinophosphonium hexafluorophosphate (PyBOP), (7-Azabenzotriazol-1-yloxy)tripyrrolidinophosphonium hexafluorophosphate (PyAOP), Bromotripyrrolidinophosphonium hexafluorophosphate (PyBrOP), Bis(2-oxo-3-oxazolidinyl)phosphinic chloride (BOP-Cl), O-(Benzotriazol-1-yl)-N,N,N′,N′-tetramethyluronium hexafluorophosphate (HBTU), O-(Benzotriazol-1-yl)-N,N,N′,N′-tetramethyluronium tetrafluoroborate (TBTU), O-(7-Azabenzotriazol-1-yl)-N,N,N′,N′-tetramethyluronium hexafluorophosphate (HATU), O-(7-Azabenzotriazol-1-yl)-N,N,N′,N′-tetramethyluronium tetrafluoroborate (TATU), O-(6-Chlorobenzotriazol-1-yl)-N,N,N′,N′-tetramethyluronium hexafluorophosphate (HCTU), Carbonyldiimidazole (CDI), and N,N,N′,N′-Tetramethylchloroformamidinium Hexafluorophosphate (TCFH).

### Attaching Protein Binding Molecules to Oligomeric Backbone

Generally the oligomeric backbone is functionalized to adapt to the type of chemical reactions can be performed to link the oligomers to the attaching position in protein binding moieties. The type reactions are suitable but not limited to, are amide coupling reactions, ether formation reactions (O-alkylation reactions), amine formation reactions (N-alkylation reactions), and sometimes carbon-carbon coupling reactions. The general reactions used to link oligomers and protein binders are shown in below schemes (VIII through X). The compounds and structures shown in Table 2 can be attached to the oligomeric backbone described herein at any position that is chemically feasible while not interfering with the hydrogen bond between the compound and the regulatory protein.

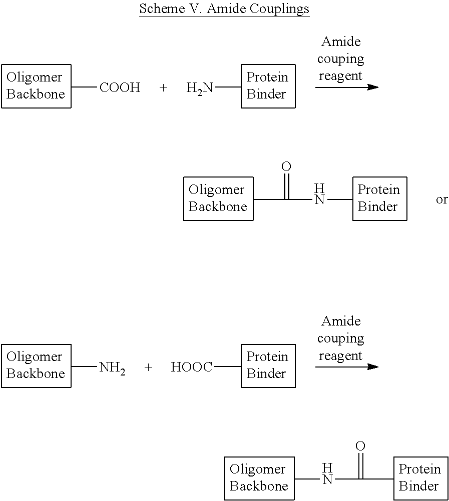

Either the oligomer or the protein binder can be functionalized to have a carboxylic acid and the other coupling counterpart being functionalized with an amino group so the moieties can be conjugated together mediated by amide coupling reagents. The amide coupling reagents can be used, but not limited to, are carbodiimides such as dicyclohexylcarbodiimide (DCC), diisopropylcarbodiimide (DIC), ethyl-(N′,N′-dimethylamino)propylcarbodiimide hydrochloride (EDC), in combination with reagents such as 1-hydroxybenzotriazole (HOBt), 4-(N,N-dimethylamino)pyridine (DMAP) and diisopropylethylamine (DIEA). Other reagents are also often used depending the actual coupling reactions are (Benzotriazol-1-yloxy)tris(dimethylamino)phosphonium hexafluorophosphate (BOP), (Benzotriazol-1-yloxy)tripyrrolidinophosphonium hexafluorophosphate (PyBOP), (7-Azabenzotriazol-1-yloxy)tripyrrolidinophosphonium hexafluorophosphate (PyAOP), Bromotripyrrolidinophosphonium hexafluorophosphate (PyBrOP), Bis(2-oxo-3-oxazolidinyl)phosphinic chloride (BOP-Cl), O-(Benzotriazol-1-yl)-N,N,N′,N′-tetramethyluronium hexafluorophosphate (HBTU), O-(Benzotriazol-1-yl)-N,N,N′,N′-tetramethyluronium tetrafluoroborate (TBTU), O-(7-Azabenzotriazol-1-yl)-N,N,N′,N′-tetramethyluronium hexafluorophosphate (HATU), O-(7-Azabenzotriazol-1-yl)-N,N,N′,N′-tetramethyluronium tetrafluoroborate (TATU), O-(6-Chlorobenzotriazol-1-yl)-N,N,N′,N′-tetramethyluronium hexafluorophosphate (HCTU), Carbonyldiimidazole (CDI), and N,N,N′,N′-Tetramethylchloroformamidinium Hexafluorophosphate (TCFH).

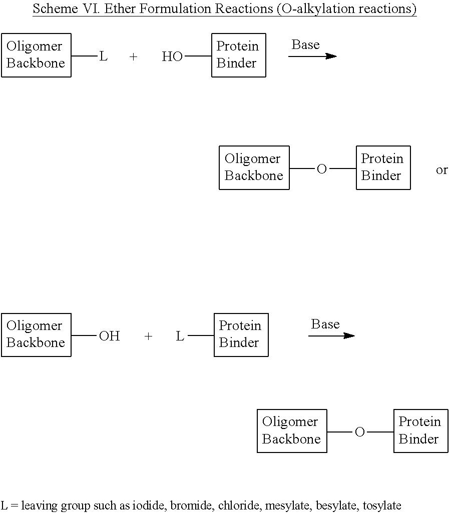

In an ether formation reaction, either the oligomer or the protein binder can be functionalized to have an hydroxyl group (phenol or alcohol) and the other coupling counterpart being functionalized with a leaving group such as halide, tosylate and mesylate so the moieties can be conjugated together mediated by a base or catalyst. The bases can be selected from, but not limited to, sodium hydride, potassium hydride, sodium hydroxide, potassium hydroxide, sodium carbonate, potassium carbonate. The catalyst can be selected from silver oxide, phase transfer reagents, iodide salts, and crown ethers.

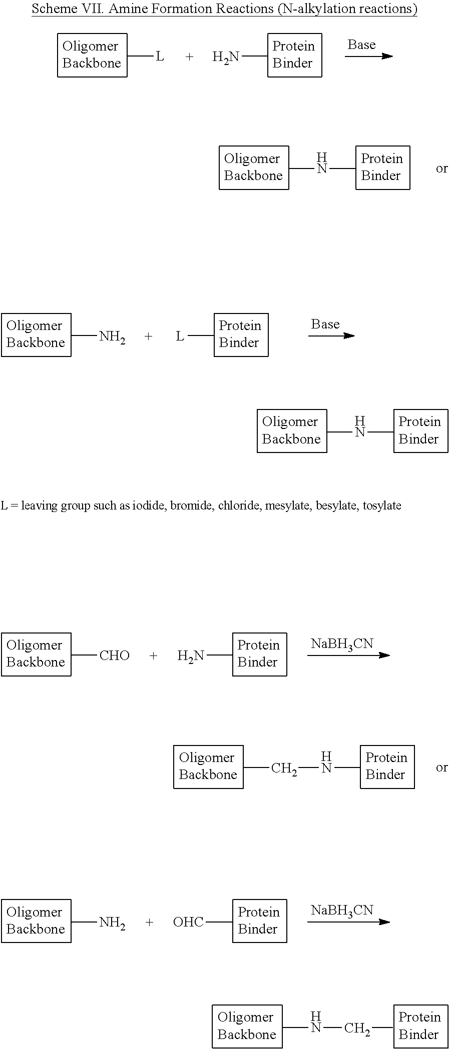

In an N-alkylation reaction, either the oligomer or the protein binder can be functionalized to have an amino group (arylamine or alkylamine) and the other coupling counterpart being functionalized with a leaving group such as halide, tosylate and mesylate so the moieties can be conjugated together directly or with a base or catalyst. The bases can be selected from, but not limited to, sodium hydride, potassium hydride, sodium hydroxide, potassium hydroxide, sodium carbonate, potassium carbonate. The catalyst can be selected from silver oxide, phase transfer reagents, iodide salts, and crown ethers. The alkylation of amines can also be achieved through reductive amination reactions, where in either the oligomer or the protein binder can be functionalized to have an amino group (arylamine or alkylamine) and the other coupling counterpart being functionalized with an aldehyde or ketone group so the moieties can be conjugated together with the treatment of a reducing reagent (hydride source) directly or in combination with a dehydration agent. The reducing reagents can be selected from, but not limited to, NaBH₄, NaHB(OAc)₃, NaBH₃CN, and dehydration agents are normally Ti(iPrO)₄, Ti(OEt)₄, Al(iPrO)₃, orthoformates and activated molecular sieves.

### Cell-Penetrating Ligand

In one aspect, the molecules of the present disclosure comprises a cell-penetrating ligand moiety. The cell-penetrating ligand moiety serves to facilitate transport of the compound across cell membranes. In certain embodiments, the cell-penetrating ligand moiety is a polypeptide. Several peptide sequences can facilitate passage into the cell, including polycationic sequences such as poly-R; arginine-rich sequences interspersed with spacers such as (RXR)ₙ (X=6-aminohexanoic acid) and (RXRRBR)ₙ (B=beta-alanine); sequences derived from the Penetratin peptide; and sequences derived from the PNA/PMO internalization peptide (Pip). The Pip5 series is characterized by the sequence ILFQY.

In certain embodiments, the cell-penetrating polypeptide comprises an N-terminal cationic sequence H₂N—(R)ₙ—CO—, with n=5-10, inclusive. In certain embodiments, the N-terminal cationic sequence contains 1, 2, or 3 substitutions of R for amino acid resides independently chosen from beta-alanine and 6-aminohexanoic acid.

In certain embodiments, the cell-penetrating polypeptide comprises the ILFQY sequence. In certain embodiments, the cell-penetrating polypeptide comprises the QFLY sequence. In certain embodiments, the cell-penetrating polypeptide comprises the QFL sequence.

In certain embodiments, the cell-penetrating polypeptide comprises a C-terminal cationic sequence —HN—(R)ₙ—COOH, with n=5-10, inclusive. In certain embodiments, the C-terminal cationic sequence contains 1, 2, or 3 substitutions of R for amino acid resides independently chosen from beta-alanine and 6-aminohexanoic acid. In certain embodiments, the C-terminal cationic sequence is substituted at every other position with an amino acid residue independently chosen from beta-alanine and 6-aminohexanoic acid. In certain embodiments, the C-terminal cationic sequence is —HN—RXRBRXRB—COOH.

TABLE 6
Cell-penetrating peptides.

| SEQ ID NO. | Sequence |
|---|---|
| SEQ ID NO. 1 | GRKKRRQRRRPPQ |
| SEQ ID NO. 2 | RQIKIWFQNRRMKWKK |
| SEQ ID NO. 3 | KLALKLALKALKAALKLA |
| SEQ ID NO. 4 | GWTLNS/AGYLLGKINLKALAALAKKIL |
| SEQ ID NO. 5 | NAKTRRHERRRKLAIER |
| SEQ ID NO. 6 | RRRRRRRR |
| SEQ ID NO. 7 | RRRRRRRRR |
| SEQ ID NO. 8 | GALFLGFLGAAGSTMGA |
| SEQ ID NO. 9 | KETWWETWWTEWSQPKKKRKV |
| SEQ ID NO. 10 | LLIILRRRIRKQAHAHSK |
| SEQ ID NO. 11 | YTAIAWVKAFIRKLRK |
| SEQ ID NO. 12 | IAWVKAFIRKLRKGPLG |
| SEQ ID NO. 13 | MVTVLFRRLRIRRACGPPRVRV |
| SEQ ID NO. 14 | GLWRALWRLLRSLWRLLWRA |
| SEQ ID NO. 15 | RRRRRRRQIKIWFQNRRMKWKKGG |
| SEQ ID NO. 16 | RXRRXRRXRIKILFQNRRMKWKK |
| SEQ ID NO. 17 | RXRRXRRXRIdKILFQNdRRMKWHKB |
| SEQ ID NO. 18 | RXRRXRRXRIHILFQNdRRMKWHKB |
| SEQ ID NO. 19 | RXRRBRRXRILFQYRXRBRXRB |
| SEQ ID NO. 20 | RXRRBRRXRILFQYRXRXRXRB |
| SEQ ID NO. 21 | RXRRXRILFQYRXRRXR |
| SEQ ID NO. 22 | RBRRXRRBRILFQYRBRXRBRB |
| SEQ ID NO. 23 | RBRRXRRBRILFQYRXRBRXRB |
| SEQ ID NO. 24 | RBRRXRRBRILFQYRXRRXRB |
| SEQ ID NO. 25 | RBRRXRRBRILFQYRXRBRXB |
| SEQ ID NO. 26 | RXRRBRRXRILFQYRXRRXRB |
| SEQ ID NO. 27 | RXRRBRRXRILFQYRXRBRXB |
| SEQ ID NO. 28 | RXRRBRRXRYQFLIRXRBRXRB |
| SEQ ID NO. 29 | RXRRBRRXRIQFLIRXRBRXRB |
| SEQ ID NO. 30 | RXRRBRRXRQFLIRXRBRXRB |
| SEQ ID NO. 31 | RXRRBRRXRQFLRXRBRXRB |
| SEQ ID NO. 32 | RXRRBRRXYRFLIRXRBRXRB |
| SEQ ID NO. 33 | RXRRBRRXRFQILYRXRBRXRB |
| SEQ ID NO. 34 | RXRRBRRXYRFRLIXRBRXRB |
| SEQ ID NO. 35 | RXRRBRRXILFRYRXRBRXRB |
| SEQ ID NO. 36 | Ac-RRLSYSRRRFXBpgG |
| SEQ ID NO. 37 | Ac-RRLSYSRRRFPFVYLIXBpgG |

Ac=acetyl; Bpg=L-bis-homopropargylglycine

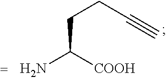

B=beta-alanine; X=6-aminohexanoic acid; dK/dR=corresponding D-amino acid.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

### Example 1. Synthesis of the Compounds

The following examples are intended to illustrate but not limit the disclosed embodiments.

Scheme A describes the steps involved for preparing the polyamide, attaching the polyamide to the oligomeric backbone, and then attaching the ligand to the other end of the oligomeric backbone. The transcription modulator molecule such as those listed in Table 8 below can be prepared using the synthesis scheme shown below.

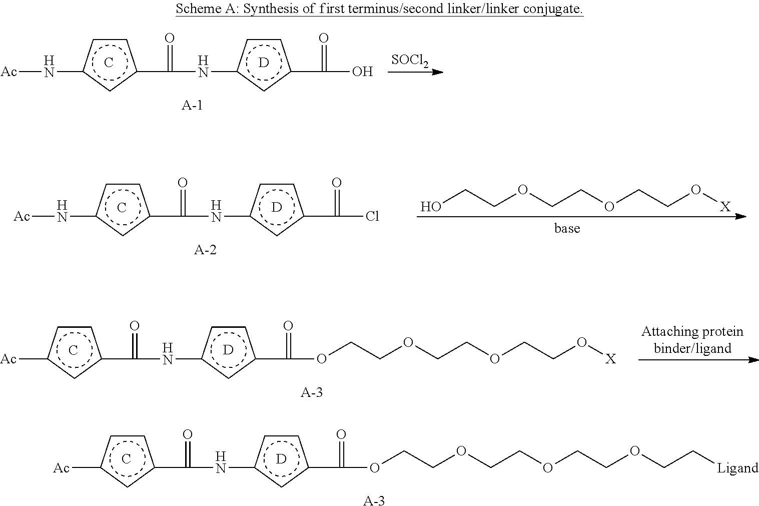

The ligand or protein binder can be attached to the oligomeric backbone using the schemes described below. The oligomeric backbone can be linked to the protein binder at any position on the protein binder that is chemically feasible while not interfering with the binding between the protein binder and the regulatory protein. The protein binder binds to the regulatory protein often through hydrogen bonds, and linking the oligomeric backbone and the regulatory protein should not interfere the hydrogen bond formation.

### Example 1A Synthesis of Polyamide Intermediates (First Terminus) and Derivatives

### Synthesis of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH)

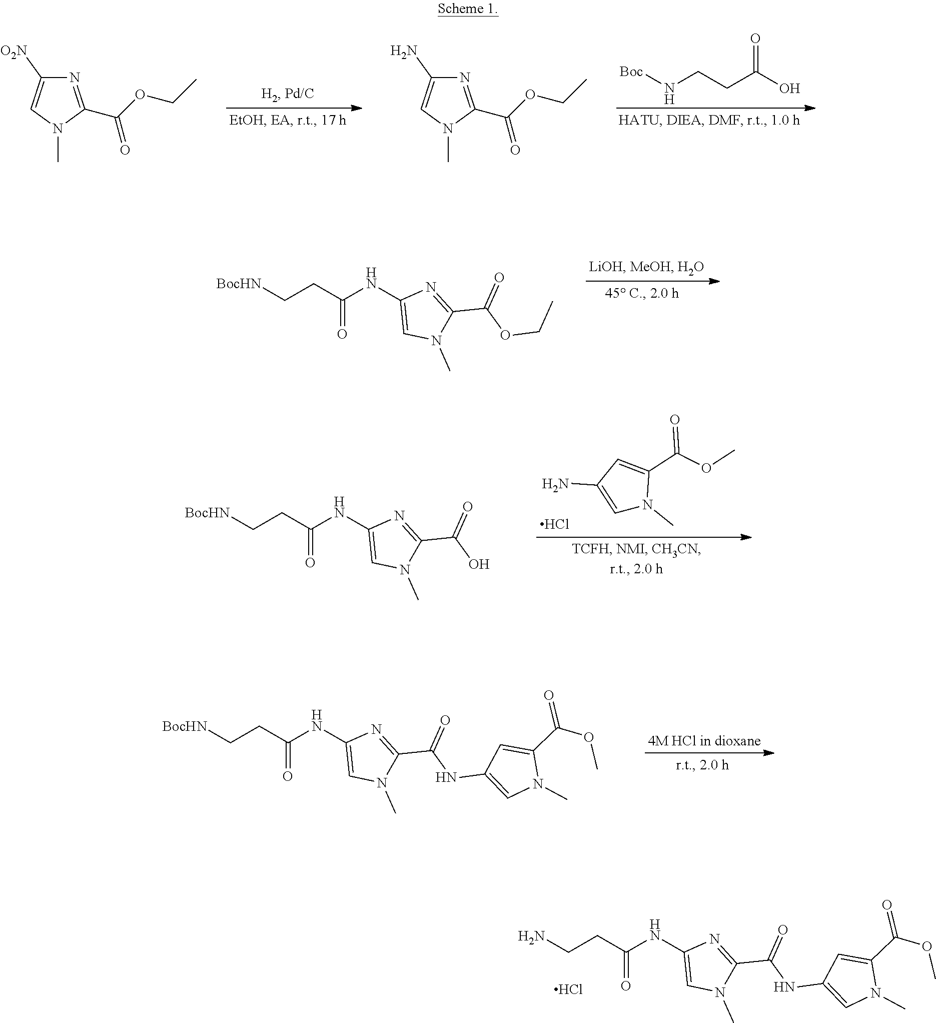

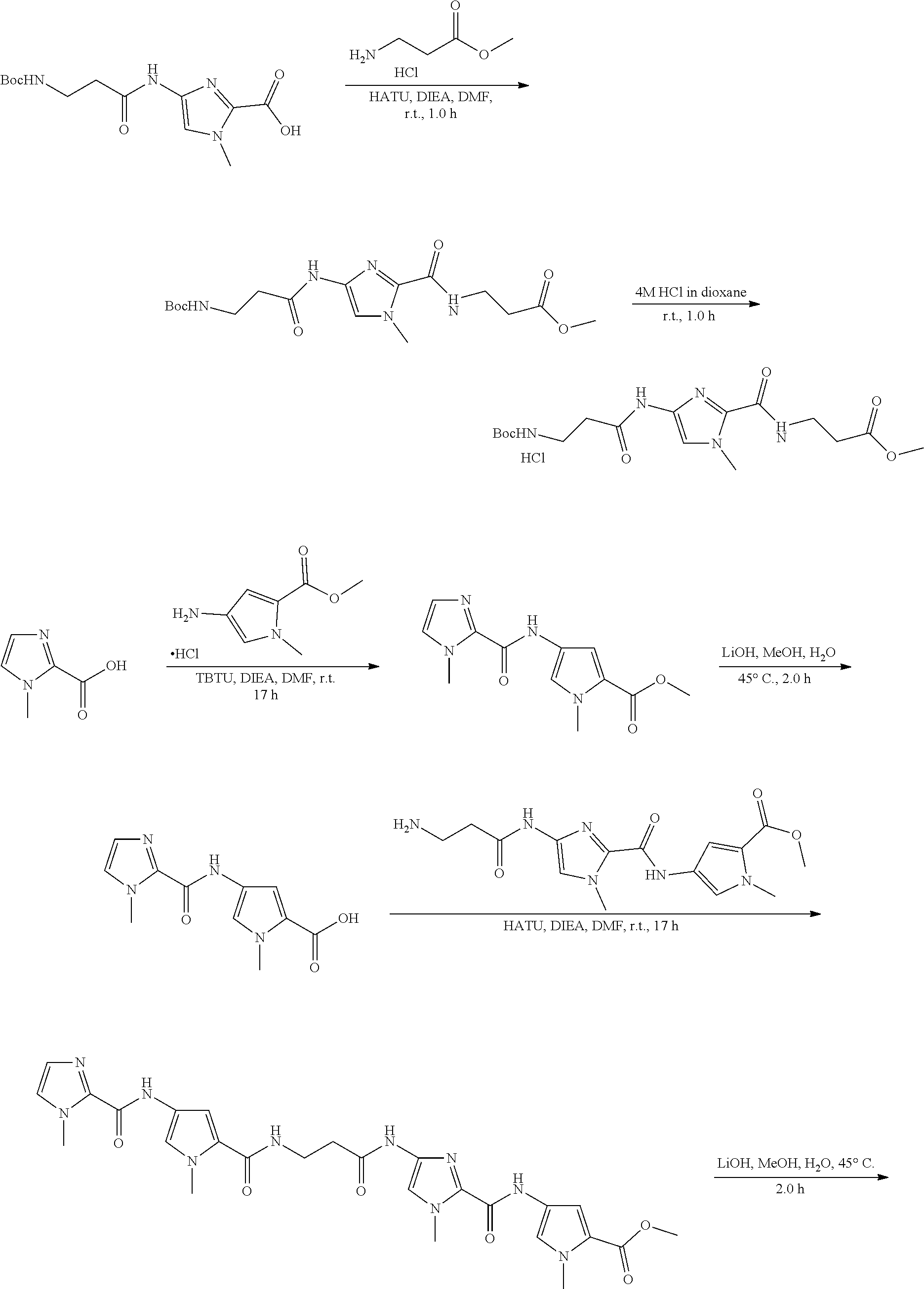

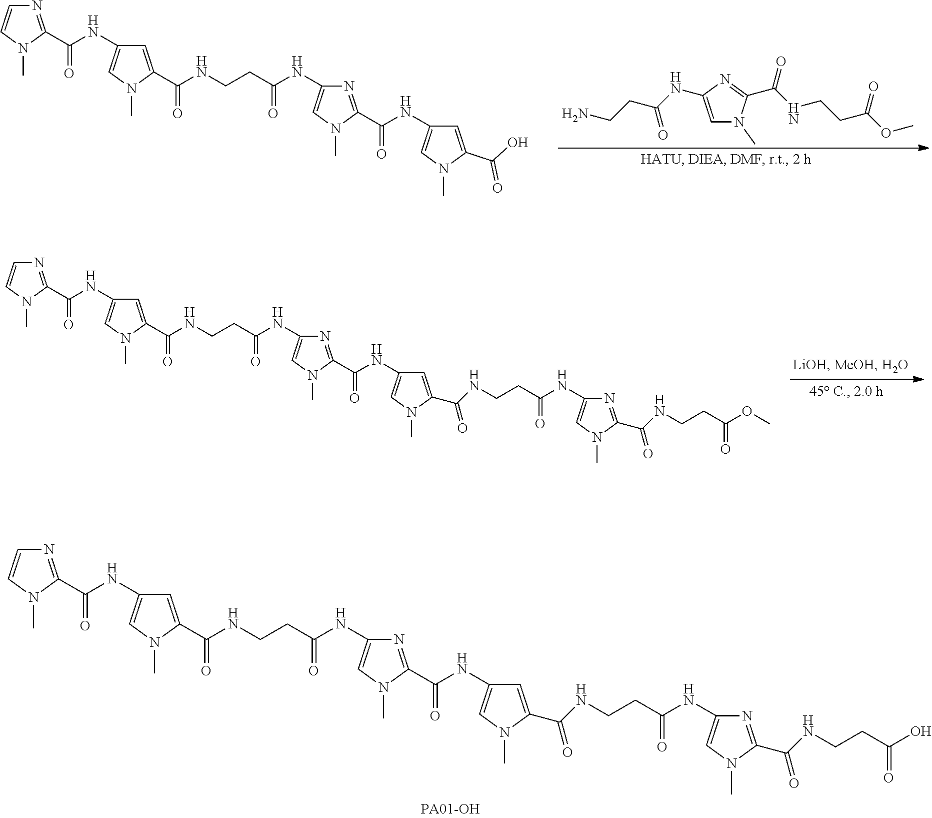 [SMILES: Cn1cc(NC(=O)c2nc(NC(=O)CCNC(=O)c3cc(NC(=O)c4nccn4C)cn3C)cn2C)cc1C(=O)O ; COC(=O)CCNC(=O)c1nc(NC(=O)CCN)cn1C ; COC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)O)n1]

### Step 1: Synthesis of ethyl 4-amino-1-methylimidazole-2-carboxylate

To a solution of ethyl 1-methyl-4-nitroimidazole-2-carboxylate (30.00 g, 150.63 mmol, 1.00 equiv) in EtOH (120.00 mL) and EA (120.00 mL) was added Pd/C (8.01 g, 27% w/w). Then the reaction was stirred for 17 h at room temperature under H₂ atmosphere. The solid was filtrated out and the filtrate was concentrated to afford ethyl 4-amino-1-methylimidazole-2-carboxylate (22.30 g, 75.20%) as yellow solid. LC/MS: mass calcd. For C₇H₁₁N₃O₂: 169.09, found: 170.10 [M+H]⁺. ¹H NMR (400 MHz, DMSO-d₆) δ: 7.37 (s, 1H), 4.29-4.34 (m, 2H), 3.94 (s, 3H), 1.31 (t, J=7.2 Hz, 3H).

### Step 2: Synthesis of ethyl 4-[3-[(tert-butoxycarbonyl)amino]propanamido]-1-methylimidazole-2-carboxylate

Into a 500 mL flask was added 3-[(tert-butoxycarbonyl) amino]propanoic acid (22.45 g, 118.65 mmol, 0.90 equiv), DMF (180.00 mL). The mixture was cooled to 0 degrees C., then HATU (75.18 g, 197.71 mmol, 1.50 equiv) and DIEA (51.11 g, 395.43 mmol, 3.00 equiv) were added, the mixture was stirred for 10 mins, then ethyl 4-amino-1-methylimidazole-2-carboxylate (22.30 g, 131.81 mmol, 1.00 equiv) was added in portions. The reaction was stirred at room temperature for 1 h. The reaction was quenched with ice water (600 mL), and the solution was stirred for 15 min. The precipitated solids were collected by filtration and washed with water (3×50 mL) and dried under vacuum. This resulted in ethyl 4-[3-[(tert-butoxycarbonyl)amino]propanamido]-1-methylimidazole-2-carboxylate (34.50 g, 76.90%) as light yellow solid. LC/MS: mass calcd. For C₁₅H₂₄N₄O₅: 340.17, found: 341.20 [M+H]⁺. ¹H NMR (400 MHz, DMSO-d₆) δ: 10.63 (s, 1H), 7.52 (s, 1H), 6.80 (t, J=5.6 Hz, 1H), 4.23-4.28 (m, 2H), 3.90 (s, 3H), 3.15-3.20 (m, 2H), 2.42 (t, J=7.2 Hz, 2H), 1.37 (s, 9H), 1.29 (t, J=7.2 Hz, 3H).

### Step 3: Synthesis of 4-[3-[(Tert-butoxycarbonyl)amino]propanamido]-1-methylimidazole-2-carboxylic acid

To a stirred solution of ethyl 4-[3-[(tert-butoxycarbonyl)amino]propanamido]-1-methylimidazole-2-carboxylate (34.50 g, 101.36 mmol, 1.00 equiv) in MeOH (200.00 mL) was added LiOH solution (2 M, 202.00 mL, 4.00 equiv) dropwise at room temperature. The resulting mixture was stirred for 2 h at 45 degree C. The resulting mixture was concentrated under reduced pressure. The residue was dissolved in H₂O (50 mL). The mixture was acidified to pH 3˜5 with 2M HCl. The precipitated solids were collected by filtration and washed with H₂O (3×30 mL), dried under vacuum. 4-[3-[(Tert-butoxycarbonyl)amino]propanamido]-1-methylimidazole-2-carboxylic acid (30.00 g, 94.77%) was obtained as white solid. LC/MS: mass calcd. For C₁₃H₂₀N₄O₅: 312.14, found: 313.15 [M+H]⁺. ¹H NMR (300 MHz, DMSO-d₆) δ: 10.53 (s, 1H), 7.48 (s, 1H), 6.79 (t, J=5.4 Hz, 1H), 3.89 (s, 3H), 3.15-3.22 (m, 2H), 2.43 (t, J=7.2 Hz, 2H), 1.37 (s, 9H).

### Step 4: Synthesis of Methyl 4-(4-[3-[(tert-butoxycarbonyl)amino]propanamido]-1-methylimidazole-2-amido)-1-methylpyrrole-2-carboxylate

To a stirred solution of 4-[3-[(tert-butoxycarbonyl)amino]propanamido]-1-methylimidazole-2-carboxylic acid (16.00 g, 51.23 mmol, 1.00 equiv) in CH₃CN (150.00 mL) was added TCFH (21.56 g, 76.84 mmol, 1.50 equiv), NMI (12.62 g, 153.69 mmol, 3.00 equiv) and methyl 4-amino-1-methylpyrrole-2-carboxylate hydrocholide (10.74 g, 56.34 mmol, 1.10 equiv) in portions at 0 degrees C. The resulting mixture was stirred for 2.0 h at room temperature. The precipitated solids were collected by filtration and washed by CH₃CN (3×20 mL), dried under vacuum. Methyl 4-(4-[3-[(tert-butoxycarbonyl)amino]propanamido]-1-methylimidazole-2-amido)-1-methylpyrrole-2-carboxylate (19.00 g, 82.70%) was obtained as white solid. LC/MS: mass calcd. For C₂₀H₂₈N₆O₆: 448.21, found: 449.25 [M+H]. ¹H NMR (300 MHz, DMSO-d₆) δ: 10.24 (s, 1H), 10.11 (s, 1H), 7.52 (s, 1H), 7.33 (s, 1H), 6.99 (s, 1H), 6.82 (t, J=5.1 Hz, 1H), 3.94 (s, 3H), 3.85 (s, 3H), 3.74 (s, 3H), 3.16-3.23 (m, 2H), 2.47 (t, J=6.9 Hz, 2H), 1.38 (s, 9H).

### Step 5: Synthesis of Methyl 4-[4-(3-aminopropanamido)-1-methylimidazole-2-amido]-1-methylpyrrole-2-carboxylate hydrochloride

A solution of methyl 4-(4-[3-[(tert-butoxycarbonyl)amino]propanamido]-1-methylimidazole-2-amido)-1-methylpyrrole-2-carboxylate (19.00 g, 42.37 mmol, 1.00 equiv) in HCl/1,4-dioxane (4M, 200.00 mL) was stirred for 2 h at room temperature. The resulting mixture was concentrated under vacuum. Methyl 4-[4-(3-aminopropanamido)-1-methylimidazole-2-amido]-1-methylpyrrole-2-carboxylate hydrochloride (19.00 g crude) was obtained as yellow solid. LC/MS: mass calcd. For C₁₅H₂₁C₁N₆O₄: 348.15, found: 349.05 [M+H]⁺. ¹H NMR (300 MHz, CD₃OD) δ: 7.37 (s, 2H), 6.91 (s, 1H), 4.03 (s, 3H), 3.88 (s, 3H), 3.79 (s, 3H), 3.09 (t, J=6.6 Hz, 2H), 2.64 (t, J=6.6 Hz, 2H).

### Step 6: Synthesis of methyl 3-[(4-[3-[(tert-butoxycarbonyl)amino] propanamido]-1-methylimidazol-2-yl)formamido]propanoate

Into a 1000 mL flask was added 4-[3-[(tert-butoxycarbonyl)amino] propanamido]-1-methylimidazole-2-carboxylic acid (11.00 g, 35.22 mmol, 1.00 equiv), DMF (300.00 mL), the mixture was cooled to 0 degrees C., then HATU (20.09 g, 52.83 mmol, 1.50 equiv), DIEA (18.21 g, 140.88 mmol, 4.00 equiv) was added dropwise, the mixture was stirred for 10 mins, methyl 3-aminopropanoate (3.63 g, 35.22 mmol, 1.00 equiv) was added in portions. The reaction was stirred at room temperature for 1 h. The reaction mixture was poured into ice/water (600 mL), the solid was filtered out and dried under vacuum. The aqueous phase was extracted by EA (3×200 mL), the organic phases were combined and washed by H₂O (1×200 mL) and NaCl (1×200 mL), dried over anhydrous Na₂SO₄. After filtration, the filtrate was concentrated under reduced pressure. The residue was purified by silica gel column, eluted with pure EA. The fractions were combined and concentrated. Methyl 3-[(4-[3-[(tert-butoxycarbonyl)amino]propanamido]-1-methylimidazol-2-yl)formamido]propanoate (13.00 g, 87.95%) was obtained as yellow solid. LC/MS: mass calcd. For C₁₇H₂₇N₅O₆: 397.20, found: 398.20 [M+H]⁺. ¹H NMR (400 MHz, DMSO-d₆) δ: 10.28 (s, 1H), 7.92 (t, J=6.0 Hz, 1H), 7.37 (s, 1H), 6.77 (t, J=6.0 Hz, 1H), 3.88 (s, 3H), 3.59 (s, 3H), 3.42-3.47 (m, 2H), 3.13-3.18 (m, 2H), 2.56 (t, J=6.0 Hz, 2H), 2.42 (t, J=6.0 Hz, 2H), 1.35 (s, 9H).

### Step 7: Synthesis of methyl 3-[[4-(3-aminopropanamido)-1-methylimidazol-2-yl]formamido]propanoate hydrochloride

A solution of methyl 3-[(4-[3-[(tert-butoxycarbonyl)amino]propanamido]-1-methylimidazol-2-yl)formamido]propanoate (11.00 g, 27.678 mmol, 1.00 equiv) in HCl/1,4-dioxane (4M, 110.00 mL) was stirred for 1.0 h at room temperature. The resulting mixture was concentrated under vacuum to afford methyl 3-[[4-(3-aminopropanamido)-1-methylimidazol-2-yl]formamido]propanoate hydrochloride (11.00 g, crude) as yellow oil. LC/MS: mass calcd. For C₁₂H₁₉N₅O₄: 297.14, found: 298.20 [M+H]⁺. ¹H NMR (400 MHz, DMSO-d₆) δ: 10.57 (s, 1H), 7.92 (t, J=6.0 Hz, 1H), 7.37 (s, 1H), 3.89 (s, 3H), 3.59 (s, 3H), 3.43-3.47 (m, 2H), 2.97-3.05 (m, 2H), 2.57-2.71 (m, 2H), 2.56 (t, J=6.0 Hz, 2H).

### Step 8: Synthesis of Methyl 1-methyl-4-(1-methylimidazole-2-amido)pyrrole-2-carboxylate

To a stirred solution of 1-methylimidazole-2-carboxylic acid (10.00 g, 79.29 mmol, 7.00 equiv) in DMF (150.00 mL) was added TBTU (38.19 g, 118.94 mmol, 1.50 equiv), methyl 4-amino-1-methylpyrrole-2-carboxylate hydrochloride (16.63 g, 87.24 mmol, 1.10 equiv) and DIEA (30.74 g, 237.88 mmol, 3.00 equiv) in portions at 0 degrees C. The resulting mixture was stirred for 17 h at room temperature. The reaction was poured into ice/water (450 mL). The precipitated solids were collected by filtration and washed with H₂O (3×50 mL), dried under vacuum. Methyl 1-methyl-4-(1-methylimidazole-2-amido)pyrrole-2-carboxylate (16.50 g, 78.37%) was obtained as white solid. LC/MS: mass calcd. For C₁₂H₁₄N₄O₃: 262.11, found: 263.15 [M+H]⁺. ¹H NMR (300 MHz, DMSO-d₆) δ: 10.54 (s, 1H), 7.54 (s, 1H), 7.40 (s, 1H), 7.04 (s, 2H), 3.99 (s, 3H), 3.85 (s, 3H), 3.74 (s, 3H).

### Step 9: Synthesis of 1-Methyl-4-(1-methylimidazole-2-amido)pyrrole-2-carboxylic acid

To a stirred solution of methyl 1-methyl-4-(1-methylimidazole-2-amido)pyrrole-2-carboxylate (16.50 g, 62.91 mmol, 1.00 equiv) in MeOH (100.00 mL) was added LiOH solution (2M, 158.00 mL, 5.00 equiv) dropwise at room temperature. The resulting mixture was stirred for 2 h at 45 degrees C. The resulting mixture was concentrated under reduced pressure. The residue was dissolved in H₂O (50 mL). The mixture was acidified to pH 3˜5 with 2M HCl. The precipitated solids were collected by filtration and washed with H₂O (3×30 mL), dried under vacuum. 1-Methyl-4-(1-methylimidazole-2-amido)pyrrole-2-carboxylic acid (12.00 g, 76.84%) was obtained as a white solid. LC/MS: mass calcd. For C₁₁H₁₂N₄O₃: 248.09, found: 249.10 [M+H]⁺. ¹H NMR (300 MHz, DMSO-d₆) δ: 10.52 (s, 1H), 7.48 (s, 1H), 7.41 (s, 1H), 7.06 (s, 1H), 6.99 (s, 1H), 3.99 (s, 3H), 3.82 (s, 3H).

### Step 10: Synthesis of Methyl 1-methyl-4-[1-methyl-4-(3-[[1-methyl-4-(1-methylimidazole-2-amido)pyrrol-2-yl]formamido]propanamido)imidazole-2-amido]pyrrole-2-carboxylate

To a stirred solution of 1-methyl-4-(1-methylimidazole-2-amido)pyrrole-2-carboxylic acid (9.00 g, 36.255 mmol, 1.00 equiv) in DMF (150.00 mL) was added HATU (20.68 g, 54.38 mmol, 1.50 equiv), DIEA (14.06 g, 108.77 mmol, 3.00 equiv) and methyl 4-[4-(3-aminopropanamido)-1-methylimidazole-2-amido]-1-methylpyrrole-2-carboxylate (13.89 g, 39.872 mmol, 1.10 equiv) in portions at 0 degrees C. The resulting mixture was stirred for 17 h at room temperature. The reaction was poured into water/Ice (450 mL) at 0 degrees C. The precipitated solids were collected by filtration and washed with H₂O (3×50 mL), dried under vacuum. Methyl 1-methyl-4-[1-methyl-4-(3-[[1-methyl-4-(1-methylimidazole-2-amido)pyrrol-2-yl]formamido]propanamido)imidazole-2-amido]pyrrole-2-carboxylate (14.00 g, 63.54%) was obtained as yellow solid. LC/MS: mass calcd. For C₂₆H₃₀N₁₀O₆: 578.23, found: 579.10 [M+H]⁺. ¹H NMR (300 MHz, DMSO-d₆) δ: 10.53 (s, 1H), 10.29 (s, 1H), 10.11 (s, 1H), 8.10 (t, J=5.4 Hz, 1H), 7.52 (s, 1H), 7.47 (s, 2H), 7.25 (s, 1H), 7.17 (s, 1H), 6.99 (s, 1H), 6.97 (s, 1H), 3.99 (s, 3H), 3.95 (s, 3H), 3.84 (s, 3H), 3.82 (s, 3H), 3.69 (s, 3H), 3.42-3.49 (m, 2H), 2.60 (t, J=7.2 Hz, 2H).

### Step 11: Synthesis of 1-methyl-4-[1-methyl-4-(3-[[1-methyl-4-(1-methylimidazole-2-amido)pyrrol-2-yl]formamido]propanamido)imidazole-2-affordamido] pyrrole-2-carboxylic acid

A solution of methyl 1-methyl-4-[1-methyl-4-(3-[[1-methyl-4-(1-methylimidazole-2-amido)pyrrol-2-yl]formamido]propanamido)imidazole-2-amido]pyrrole-2-yl]formamidocarboxylate (14.00 g, 24.20 mmol, 1.00 equiv) in MeOH (70.00 mL) was added LiOH (2M, 72.00 mL, 6.00 equiv). The mixture was stirred at 45 degrees C. for 2 h. The resulting mixture was concentrated under reduced pressure. The residue was dissolved in H₂O (50 mL). The mixture was acidified to pH 3˜5 with 2M HCl. The precipitated solids were collected by filtration and washed with H₂O (3×20 mL), dried under vacuum. 1-methyl-4-[1-methyl-4-(3-[[1-methyl-4-(1-methylimidazole-2-amido)pyrrol-2-yl]formamido]propanamido)imidazole-2-affordamido] pyrrole-2-carboxylic acid (12.00 g, 81.49%) was obtained as yellow solid. LC/MS: mass calcd. For C₂₅H₂₈N₁₀O₆: 564.22, found: 565.15 [M+H]⁺. ¹H NMR (300 MHz, DMSO-d6) δ: 10.72 (s, 1H), 10.32 (s, 1H), 10.08 (s, 1H), 8.14 (t, J=6.0 Hz, 1H), 7.51 (s, 1H), 7.47 (s, 2H), 7.27 (s, 1H), 7.23 (s, 1H), 6.98 (s, 1H), 6.94 (s, 1H), 4.00 (s, 3H), 3.95 (s, 3H), 3.82 (s, 6H), 3.44-3.46 (m, 2H), 2.60 (t, J=6.6 Hz, 2H).

### Step 12: Synthesis of Methyl 3-([1-methyl-4-[3-([1-methyl-4-[1-methyl-4-(3-[[1-methyl-4-(1-methylimidazole-2-amido)pyrrol-2-yl]formamido] propanamido)imidazole-2-amido]pyrrol-2-yl]formamido)propanamido]imidazol-2-yl]formamido)propanoate

To a stirred solution of 1-methyl-4-[1-methyl-4-(3-[[1-methyl-4-(1-methylimidazole-2-amido)pyrrol-2-yl]formamido]propanamido)imidazole-2-amido] pyrrole-2-carboxylic acid (12.00 g, 21.26 mmol, 1.00 equiv) in DMF (100.00 mL) was added HATU (12.12 g, 31.88 mmol, 1.50 equiv), DIEA (8.24 g, 63.77 mmol, 3.00 equiv) and methyl 3-[[4-(3-aminopropanamido)-1-methylimidazol-2-yl]formamido]propanoate (6.95 g, 23.38 mmol, 1.10 equiv) in portions at 0 degrees C. The resulting mixture was stirred for 2 h at room temperature. The reaction was poured into ice/water (300 mL) at 0 degrees C. The precipitated solids were collected by filtration and washed with H₂O (3×30 mL), dried under vacuum. Methyl 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoate (13.00 g, 64.77%) was obtained as yellow solid. LC/MS: mass calcd. For C₃₋₇H₄₅N₁₅O₉: 843.35, found: 844.55 [M+H]⁺. ¹H NMR (300 MHz, DMSO-d6) δ: 10.41 (s, 1H), 10.37 (s, 1H), 10.32 (s, 1H), 9.96 (s, 1H), 8.08 (s, 2H), 7.96 (s, 1H), 7.46 (s, 1H), 7.42 (s, 1H), 7.38 (s, 1H), 7.24 (s, 2H), 7.03 (s, 1H), 6.98 (s, 1H), 6.93 (s, 1H), 4.13 (s, 3H), 3.98 (s, 3H), 3.95 (s, 3H), 3.81 (s, 9H), 3.60 (s, 6H), 2.57-2.69 (m, 6H).

### Step 13: Synthesis of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid

A solution of methyl 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoate (10.00 g, 10.59 mmol, 1.00 equiv) in MeOH (60.00 mL) was added 2M LiOH (21.20 mL, 42.40 mmol, 4.00 equiv), the resulting mixture was stirred for 2 h at 45 degrees C. The resulting mixture was concentrated under reduced pressure. The resulting mixture was diluted with water (60 mL). The mixture was acidified to pH 3˜5 with 2M HCl. The precipitated solids were collected by filtration and washed with water (3×20 mL). The solid was dried under vacuum. This resulted in 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (8.70 g, 84.14%) as a brown solid. LC/MS: mass calcd. For C₃₆H₄₃N₁₅O₉: 829.34, found: 830.25 [M+H]⁺. ¹H NMR (300 MHz, DMSO-d₆) δ: 10.46 (s, 1H), 10.39 (s, 1H), 10.31 (s, 1H), 9.93 (s, 1H), 8.05-8.10 (m, 2H), 7.87 (t, J=6.0 Hz, 1H), 7.42-7.46 (m, 3H), 7.20-7.23 (m, 2H), 7.07 (s, 1H), 6.90-6.95 (m, 2H), 3.95 (s, 3H), 3.92 (s, 3H), 3.89 (s, 3H), 3.79 (s, 3H), 3.78 (s, 3H), 3.38-3.41 (m, 6H), 2.44-2.59 (m, 6H).

### Synthesis of N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA)

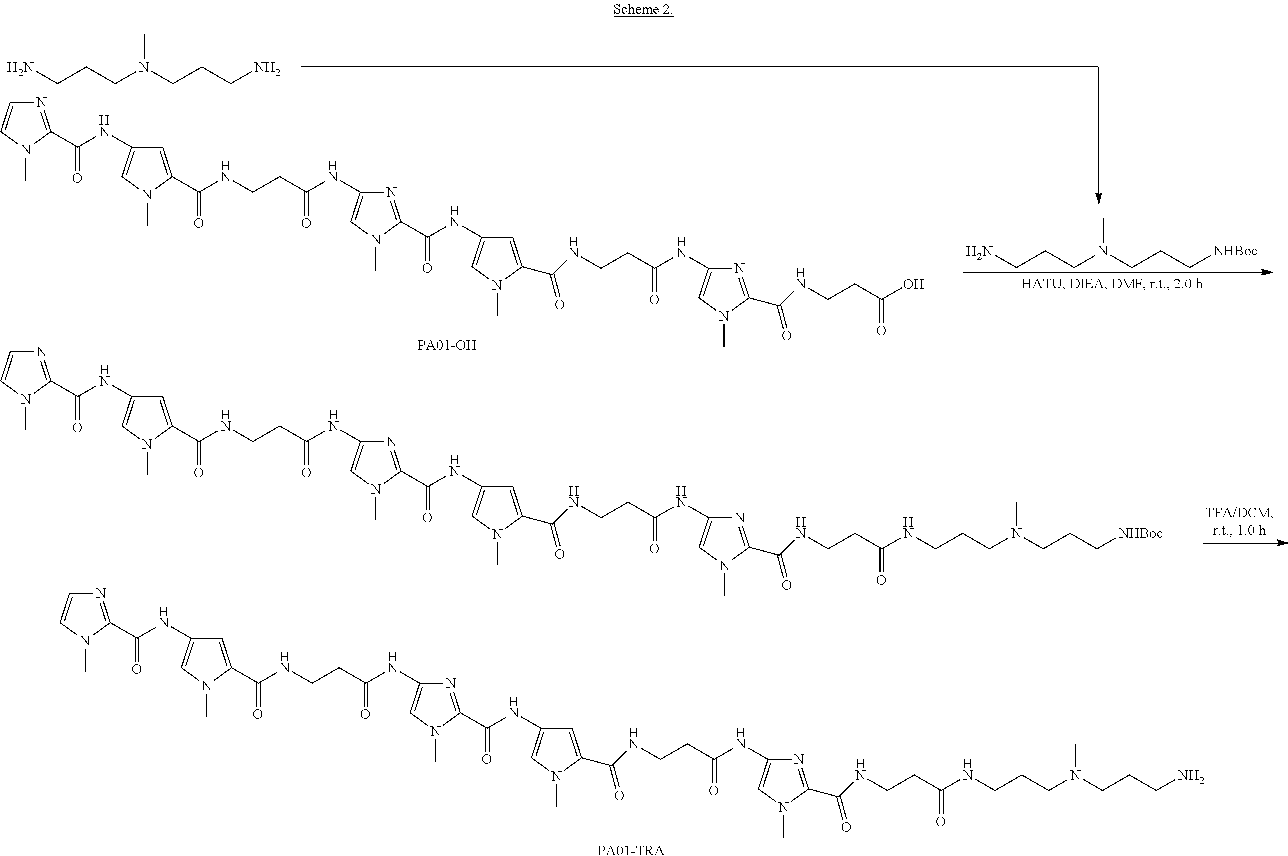

### Step 1: Synthesis of tert-butyl (3-((3-aminopropyl)(methyl)amino)propyl) carbamate

To a solution of bis(3-aminopropyl)(methyl)amine (30.00 g, 206.54 mmol, 2.00 equiv) in DCM (100.00 mL) was added dropwise a solution of Boc₂O (22.40 g, 102.64 mmol, 1.00 equiv) in DCM (100.00 mL) during 4.0 h at 0 degree C. Then the mixture was stirred at room temperature overnight. The solid was filtered out and the filtrate was concentrated. The crude was dissolved in CH₃CN (10 mL) and purified by reverse phase column: column, C18 column; mobile phase, CH₃CN in water (0.05% NH₄HCO₃), from 5% to 30% gradient in 2.0 h, detector UV 220 nm. The fractions were combined and concentrated. Tert-butyl (3-((3-aminopropyl)(methyl)amino)propyl)carbamate (18.00 g, 64.00%) was obtained as colorless oil. LC/MS: mass calcd. For C₁₂H₂₇N₃O₂: 245.21, found: 246.15 [M+H]⁺. ¹H NMR (400 MHz, DMSO) δ: 6.80 (t, J=5.2 Hz, 1H), 3.57-3.60 (m, 2H), 2.87-2.92 (m, 2H), 2.19-2.26 (m, 4H), 2.06 (s, 3H), 1.41-1.52 (m, 4H), 1.35 (s, 9H).

### Step 2: Synthesis of tert-butyl (3-(methyl(3-(3-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamido)propanamido)propyl)amino)propyl)carbamate

To a solution of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (2.50 g, 3.37 mmol, 1.00 equiv) in DMF (100.00 mL) was added tert-butyl N-[3-[(3-aminopropyl)(methyl)amino]propyl]carbamate (1.11 g, 4.52 mmol, 1.50 equiv) and HATU (1.72 g, 4.52 mmol, 1.50 equiv). Then DIEA (1.17 g, 9.04 mmol, 3.00 equiv) was added. The mixture was stirred at room temperature for 2.0 h. The mixture was poured into 200 mL ice/water, the solid was filtered out and the filter cake was washed by H₂O (20 mL), dried under high vacuum. Tert-butyl (3-(methyl(3-(3-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamido)propanamido)propyl)amino)propyl)carbamate (2.80 g, 79.12%) was obtained as yellow solid. LC/MS: mass calcd. For C₄₈H₆₈N₁₈O₁₀: 1056.54, found: 1057.80 [M+H]⁺.

### Step 3: Synthesis of N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA)

To a solution of tert-butyl N-[3-[methyl([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl])amino]propyl]carbamate (2.80 g, 2.65 mmol, 1.00 equiv) in DCM (30.00 mL) was added TFA (10.00 mL). The reaction mixture was stirred at room temperature for 1.0 h. Then the solvent was removed and the residue was lyophilized. N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (2.80 g, 93.89%) was obtained as dark yellow solid. LC/MS: mass calcd. For C₄₃H₆₀N₁₈O₈: 956.48, found: 957.70 [M+H]⁺.

### Synthesis of N-[5-[(2-[[2-([2-[(2-aminoethyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide (PA01-EDA)

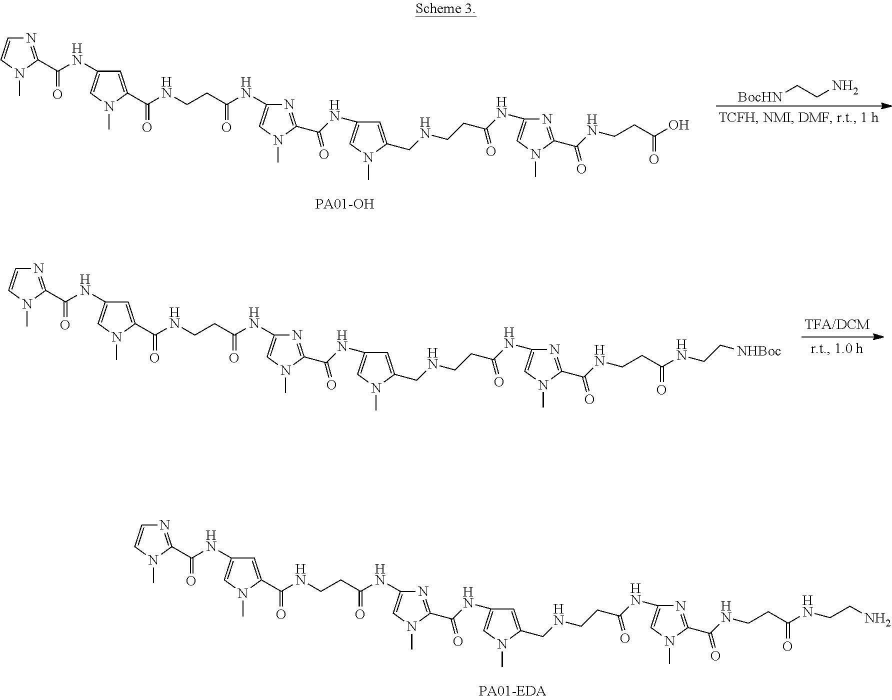 [SMILES: Cn1cc(NC(=O)c2nc(NC(=O)CCNC(=O)c3cc(NC(=O)c4nccn4C)cn3C)cn2C)cc1CNCCC(=O)Nc1cn(C)c(C(=O)NCCC(=O)O)n1 ; CC(C)(C)OC(=O)NCCN ; Cn1cc(NC(=O)c2nc(NC(=O)CCNC(=O)c3cc(NC(=O)c4nccn4C)cn3C)cn2C)cc1CNCCC(=O)Nc1cn(C)c(C(=O)NCCC(=O)NCCNC(=O)OC(C)(C)C)n1 ; Cn1cc(NC(=O)c2nc(NC(=O)CCNC(=O)c3cc(NC(=O)c4nccn4C)cn3C)cn2C)cc1CNCCC(=O)Nc1cn(C)c(C(=O)NCCC(=O)NCCN)n1]

### Step 1: Synthesis of tert-butyl N-[2-[3-([1-methyl-4-[3-([1-methyl-4-[1-methyl-4-(3-[[1-methyl-4-(1-methylimidazole-2-amido)pyrrol-2-yl] formamido]propanamido)imidazole-2-amido]pyrrol-2-yl]formamido)propanamido]imidazol-2-yl]formamido) propanamido]ethyl]carbamate

The procedure was the same as methyl 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrole-2-carboxylate. 150.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido] imidazol-2-yl]formamido)propanoic acid was used, 120.00 mg of tert-butyl N-[2-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]ethyl]carbamate was obtained as yellow oil (66.73% yield). LC/MS: mass calcd. For C₄₃H₅₇N₁₇O₁₀: 971.45, found: 487.05 [½M+H]⁺.

### Step 2: Synthesis of N-[5-[(2-[[2-([2-[(2-aminoethyl)carbamoyl]ethyl] carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide (PA01-EDA)

The procedure was the same as N-(3-((3-((3-aminopropyl)(methyl)amino)propyl) amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide. 110.00 mg of tert-butyl N-[2-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]ethyl]carbamate was used, 100.00 mg of N-[5-[(2-[[2-([2-[(2-aminoethyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as yellow oil (81.08% yield). LC/MS: mass calcd. For C₃₈H₄₉N₁₇O₈: 871.40, found: 894.60 [M+Na]⁺.

### Synthesis of N-[5-([2-[(2-[[2-([3-[(3-aminopropyl)(2,2,2-trifluoroethyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide (PA01-TRA(CH2CF3))

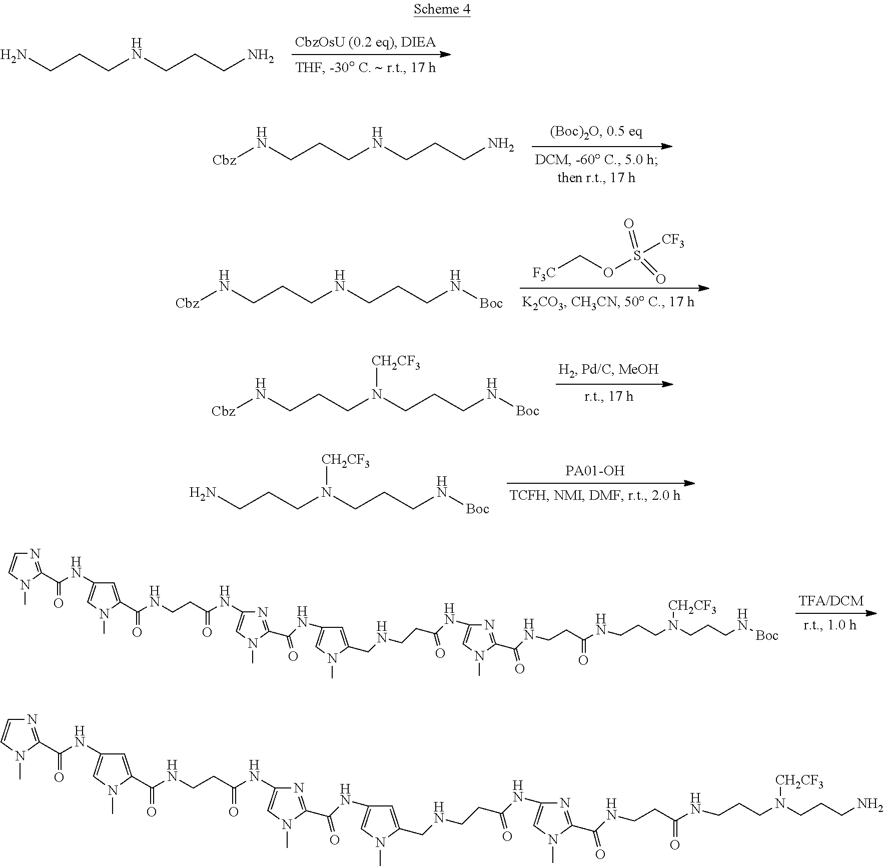

### Step 1: Synthesis of Benzyl N-[3-[(3-aminopropyl)amino]propyl]carbamate

To a stirred solution of norspermidine (6.00 g, 45.72 mmol, 1.00 equiv) in THF (50.00 mL) was added DIEA (5.91 g, 45.72 mmol, 1.00 equiv). Benzyl 2,5-dioxopyrrolidin-1-yl carbonate (2.28 g, 9.15 mmol, 0.20 equiv) in THF (30.00 mL) was added dropwise at −30 degrees C. The resulting mixture was stirred for 17 h at room temperature. The resulting mixture was filtered, the filter cake was washed with THF (3×10 mL). The filtrate was concentrated under reduced pressure. The residue was purified by reverse flash chromatography with the following conditions: column, C18 column; mobile phase, CH₃CN in water (0.05% TFA), 15% to 30% gradient in 10 min; detector, UV 254 nm. The fractions were combined and concentrated. Benzyl N-[3-[(3-aminopropyl)amino]propyl]carbamate (1.50 g, 60.00%) was obtained as yellow oil. LC/MS: mass calcd. For C₁₄H₂₃N₃O₂: 265.18, found: 266.15 [M+H]⁺.

### Step 2: Synthesis of Benzyl N-[3-([3-[(tert-butoxycarbonyl)amino]propyl]amino)propyl]carbamate

To a stirred solution of benzyl N-[3-[(3-aminopropyl)amino]propyl]carbamate (1.40 g, 5.28 mmol, 1.00 equiv) in DCM (20.00 mL) was added (Boc)₂O (0.58 g, 2.66 mmol, 0.50 equiv) in DCM (10.00 mL) solution dropwise at −60 degrees C. The resulting mixture was stirred for 5 h at −60 degrees C. and warmed to room temperature and stirred for additional 12 h at room temperature. The reaction mixture was concentrated under reduced pressure. The residue was purified by reverse flash chromatography with the following conditions: column, C18 column; mobile phase, CH₃CN in water (0.05% NH₄HCO₃), 30% to 50% gradient in 20 min; detector, UV 220 nm. The fractions were combined and concentrated. Benzyl N-[3-([3-[(tert-butoxycarbonyl)amino]propyl]amino)propyl]carbamate (500 mg, 23.63%) was obtained as yellow oil. LC/MS: mass calcd. For C₁₉H₃₁N₃O₄: 365.23, found: 366.10 [M+H]⁺.

### Step 3: Synthesis of Benzyl N-[3-([3-[(tert-butoxycarbonyl)amino]propyl] (2,2,2-trifluoroethyl)amino)propyl]carbamate

To a stirred solution of benzyl N-[3-([3-[(tert-butoxycarbonyl)amino]propyl]amino)propyl]carbamate (280.00 mg, 0.77 mmol, 1.00 equiv) in CH₃CN (8.00 mL) was added 2,2,2-trifluoroethyl trifluoromethanesulfonate (177.82 mg, 0.77 mmol, 1.00 equiv) and K₂CO₃ (317.65 mg, 2.30 mmol, 3.00 equiv) in portions at room temperature. The resulting mixture was stirred for 17 h at 50 degrees C. The solid was filtered out and the filtrate was concentrated. The residue was purified by reverse flash chromatography with the following conditions: column, C18 silica gel; mobile phase, ACN in water (0.05% NH₄HCO₃), 20% to 30% gradient in 100 min; detector, UV 254 nm. The fractions were combined and concentrated. Benzyl N-[3-([3-[(tert-butoxycarbonyl)amino]propyl](2,2,2-trifluoroethyl)amino)propyl]carbamate (150 mg, 43.75%) was obtained as colorless oil. LC/MS: mass calcd. For C₂₁H₃₂F₃N₃O₄: 447.23, found: 448.20 [M+H]⁺.

### Step 4: Synthesis of Tert-butyl N-[3-[(3-aminopropyl)(2,2,2-trifluoroethyl) amino]propyl]carbamate

To a solution of benzyl N-[3-([3-[(tert-butoxycarbonyl)amino]propyl](2,2,2-trifluoroethyl)amino)propyl]carbamate (150.00 mg, 0.34 mmol, 1.00 equiv) in MeOH (8.00 mL) was added Pd/C (30.00 mg, 20% w/w). Then the reaction was stirred for 17 h at room temperature under H₂ atmosphere. The resulting mixture was filtered and the filter cake was washed with MeOH (3×5 mL). The filtrate was concentrated under reduced pressure. Tert-butyl N-[3-[(3-aminopropyl)(2,2,2-trifluoroethyl)amino] propyl]carbamate (90.00 mg, 85.68%) was obtained as colorless oil. LC/MS: mass calcd. For C₁₃H₂₆F₃N₃O₂: 313.20, found: 314.15 [M+H]⁺.

### Step 5: Synthesis of Tert-butyl N-[3-([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl](2,2,2-trifluoroethyl)amino)propyl]carbamate

The procedure was the same as ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate. 212.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH) was used, 150.00 mg of tert-butyl N-[3-([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl](2,2,2-trifluoroethyl)amino)propyl]carbamate was obtained as yellow solid (42.94% yield). LC/MS: mass calcd. For C₄₉H₆₇F₃N₁₈O₁₀: 1124.52, found: 1125.45 [M+H]⁺.

### Step 6: Synthesis of N-[5-([2-[(2-[[2-([3-[(3-aminopropyl) (2,2,2-trifluoroethyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as N-(3-((3-((3-aminopropyl)(methyl)amino)propyl) amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA). 150.00 mg of tert-butyl N-[3-([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido] imidazol-2-yl]formamido)propanamido]propyl](2,2,2-trifluoroethyl)amino)propyl]carbamate was used, 150.00 mg crude of N-[5-([2-[(2-[[2-([3-[(3-aminopropyl) (2,2,2-trifluoroethyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as yellow oil. LC/MS: mass calcd. For C₄₄H₅₉F₃N₁₈O₈: 1024.47, found: 1025.35 [M+H]⁺.

Synthesis of 1-methyl-4-(3-[[1-methyl-4-(1-methylimidazole-2-amido) pyrrol-2-yl]formamido]propanamido)-N-[1-methyl-5-[(2-[[1-methyl-2-([2-[(3-[methyl[3-(methylamino)propyl]amino]propyl)carbamoyl]ethyl]carbamoyl)imidazol-4-yl]carbamoyl]ethyl)carbamoyl]pyrrol-3-yl]imidazole-2-carboxamide (PA01-TRA(CH₃))

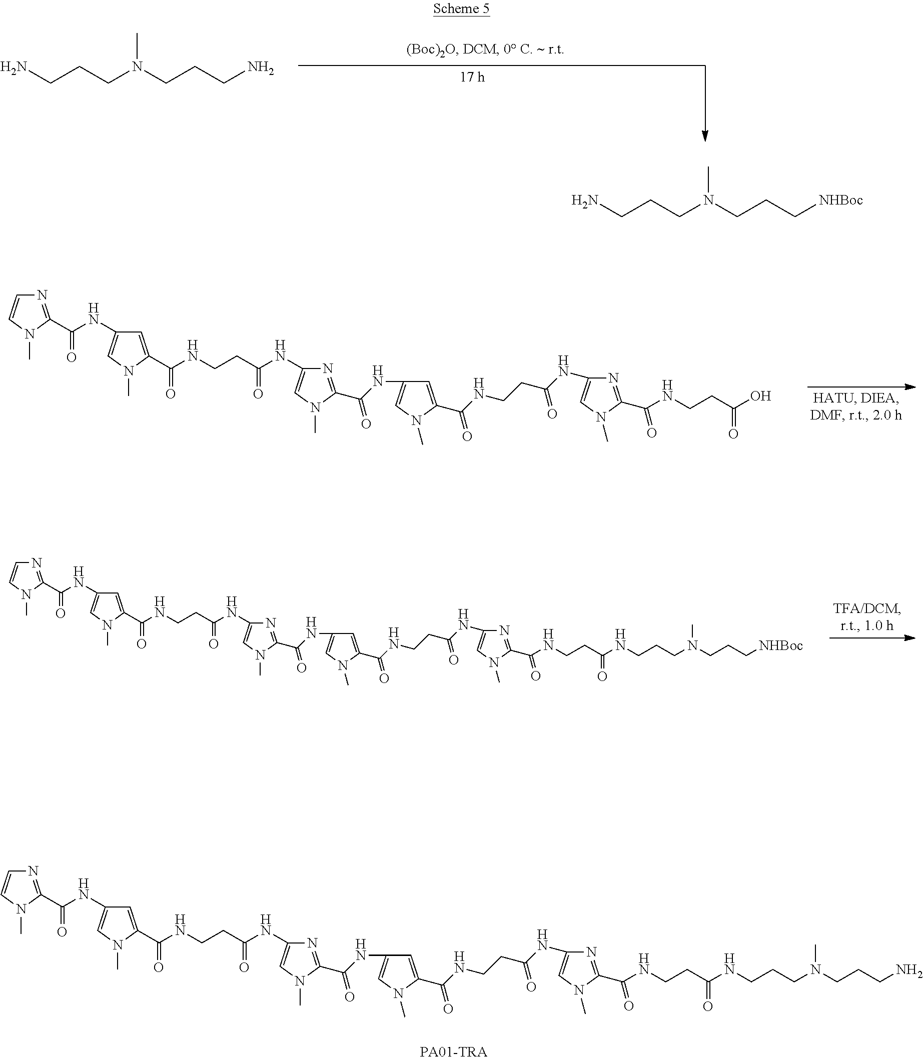

### Step 1: Synthesis of tert-butyl N-methyl-N-[3-(methylamino)propyl] carbamate

To a stirred solution of methyl[3-(methylamino)propyl]amine (2000.00 mg, 19.57 mmol, 1.00 equiv) in DCM (4.00 mL) was added (Boc)₂O (2135.89 mg, 9.79 mmol, 0.50 equiv) dropwise at 0 degrees C. The resulting mixture was stirred for 17 h at room temperature. After that, the reaction was quenched with water (30 mL) and extracted with DCM (3×50 mL). The organic layers were combined, dried over anhydrous sodium sulfate, filtered and concentrated. Tert-butyl N-methyl-N-[3-(methylamino)propyl]carbamate (2500.00 mg, 63.14%) was obtained as yellow oil. LC/MS: mass calcd. For C₁₀H₂₂N₂O₂: 202.18, found: 203.20 [M+H]⁺.

### Step 2: Synthesis of tert-butyl N-(3-[[3-(1,3-dioxoisoindol-2-yl)propyl](methyl) amino]propyl)-N-methylcarbamate

To a stirred solution of tert-butyl N-methyl-N-[3-(methylamino)propyl]carbamate (2500.00 mg, 12.36 mmol, 1.00 equiv) in CH₃CN (100.00 mL) was added N-(3-bromopropyl)phthalimide (3313.31 mg, 12.36 mmol, 1.00 equiv) and K₂CO₃ (5123.83 mg, 37.07 mmol, 3.00 equiv) in portions at room temperature. The resulting mixture was stirred for 17 h at 70 degrees C. After cooling down to room temperature, the solid was filtered out and the filtrate was concentration under reduced pressure. The residue obtained was purified by silica gel chromatography (DCM:MeOH=10:1) to afford tert-butyl N-(3-[[3-(1,3-dioxoisoindol-2-yl) propyl](methyl)amino]propyl)-N-methylcarbamate (2600.00 mg, 54.02%) as yellow oil. LC/MS: mass calcd. For C₂₁H₃₁N₃O₄: 389.23, found: 390.20 [M+H]⁺.

### Step 3: Synthesis of tert-butyl N-[3-[(3-aminopropyl)(methyl)amino]propyl]-N-methylcarbamate

To a stirred solution of tert-butyl N-(3-[[3-(1,3-dioxoisoindol-2-yl)propyl](methyl)amino]propyl)-N-methylcarbamate (2500.00 mg, 6.42 mmol, 1.00 equiv) in MeOH (30.00 mL) was added hydrazine (1028. 44 mg, 32.09 mmol, 5.00 equiv) dropwise at room temperature. The resulting mixture was stirred for 3 h at 60 degrees C. After that, the reaction was quenched with water (40 mL) and extracted with ethyl acetate (3×60 mL). The organic layers were combined, dried over anhydrous sodium sulfate, filtered and concentrated. Tert-butyl N-[3-[(3-aminopropyl)(methyl)amino]propyl]-N-methylcarbamate (2.30 g, crude) was obtained as yellow oil. LC/MS: mass calcd. For C₁₃H₂₉N₃O₂:259.23, found: 260.20 [M+H]⁺.

### Step 4: Synthesis of tert-butyl N-methyl-N-[3-[methyl([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl])amino]propyl]carbamate

The procedure was the same as tert-butyl (3-(methyl(3-(3-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamido)propanamido)propyl)amino)propyl)carbamate. 300.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido] imidazol-2-yl]formamido)propanoic acid (PA01-OH) was used, 200.00 mg of tert-butyl N-methyl-N-[3-[methyl([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl])amino]propyl]carbamate was obtained as yellow oil (42.86% yield). LC/MS: mass calcd. For C₄₉H₇₀N₁₈O₁₀: 1070.55, found: 1071.75 [M+H]⁺.

### Step 5: Synthesis of 1-methyl-4-(3-[[1-methyl-4-(1-methylimidazole-2-amido)pyrrol-2-yl]formamido]propanamido)-N-[1-methyl-5-[(2-[[1-methyl-2-([2-[(3-[methyl[3-(methylamino)propyl]amino]propyl)carbamoyl]ethyl]carbamoyl) imidazol-4-yl]carbamoyl]ethyl)carbamoyl]pyrrol-3-yl]imidazole-2-carboxamide

The procedure was the same as N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA). 90.00 mg of tert-butyl N-methyl-N-[3-[methyl([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl])amino]propyl]carbamate was used, 100.00 mg of 1-methyl-4-(3-[[1-methyl-4-(1-methylimidazole-2-amido)pyrrol-2-yl]formamido]propanamido)-N-[1-methyl-5-[(2-[[1-methyl-2-([2-[(3-[methyl[3-(methylamino)propyl]amino]propyl)carbamoyl]ethyl]carbamoyl)imidazol-4-yl]carbamoyl]ethyl)carbamoyl]pyrrol-3-yl]imidazole-2-carboxamide was obtained as yellow oil. LC/MS: mass calcd. For C₄₄H₆₂N₁₈O₈: 970.50, found: 971.75 [M+H]⁺.

### Synthesis of 1-methyl-4-(3-[[1-methyl-4-(1-methylimidazole-2-amido) pyrrol-2-yl]formamido]propanamido)-N-[1-methyl-5-[(2-[[1-methyl-2-([2-[methyl(3-[methyl[3-(methylamino)propyl]amino]propyl)carbamoyl]ethyl]carbamoyl)imidazol-4-yl]carbamoyl]ethyl)carbamoyl]pyrrol-3-yl]imidazole-2-carboxamide (PA01-TRA(diCH3)

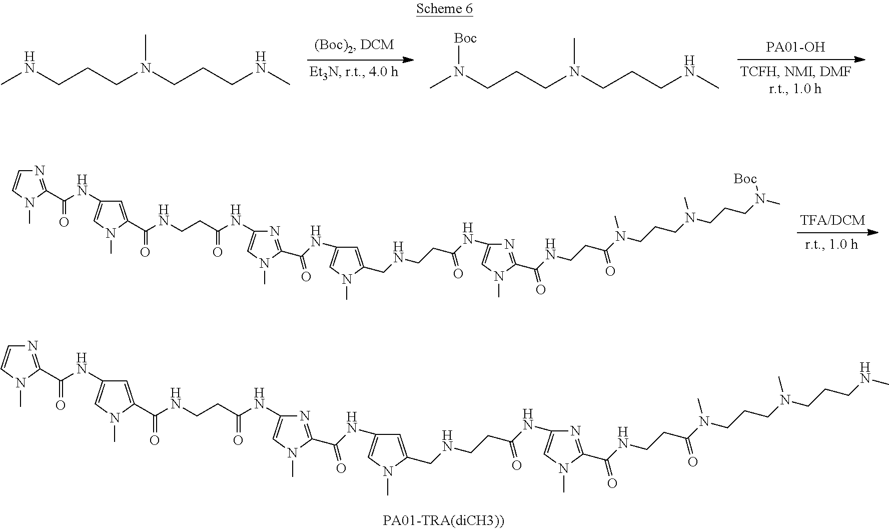

### Step 1: Synthesis of tert-butyl N-methyl-N-(3-[methyl[3-(methylamino)propyl]amino]propyl) carbamate

The procedure was the same as tert-butyl (3-((3-aminopropyl)(methyl)aminopropyl)carbamate. 1.40 g of methyl(3-[methyl[3-(methylamino)propyl]amino]propyl)amine was used, 350.00 mg of tert-butyl N-methyl-N-(3-[methyl[3-(methylamino)propyl]amino]propyl)carbamate was obtained as yellow oil (15.85% yield). LC/MS: mass calcd. For: C₁₄H₃₁N₃O₂: 273.24, found: 274.10 [M+H]⁺.

### Step 2: Synthesis of tert-butyl N-methyl-N-[3-[methyl([3-[N-methyl-3-([1-methyl-4-[3-([1-methyl-4-[1-methyl-4-(3-[[1-methyl-4-(1-methylimidazole-2-amido)pyrrol-2-yl]formamido])imidazole-2-amido]pyrrol-2-yl]formamido)propanamido]imidazol-2-yl]formamido)propanamido]propyl])amino]propyl]carbamate

The procedure was the same as ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate. 300.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH) was used, 150.00 mg of tert-butyl N-methyl-N-[3-[methyl([3-[N-methyl-3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl])amino]propyl]carbamate was obtained as yellow solid (38.10% yield). LC/MS: mass calcd. For C₅₀H₇₂N₁₈O₁₀: 1084.57, found: 543.70 [M/2+H]⁺.

### Step 3: Synthesis of 1-methyl-4-(3-[[1-methyl-4-(1-methylimidazole-2-amido)pyrrol-2-yl]formamido]propanamido)-N-[1-methyl-5-[(2-[[1-methyl-2-([2-[methyl(3-[methyl[3-(methylamino)propyl]amino]propyl)carbamoyl]ethyl]carbamoyl)imidazol-4-yl]carbamoyl]ethyl)carbamoyl]pyrrol-3-yl]imidazole-2-carboxamide

The procedure was the same as N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA). 150.00 mg of tert-butyl N-methyl-N-[3-[methyl([3-[N-methyl-3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl])amino]propyl]carbamate was used, 150.00 mg crude of 1-methyl-4-(3-[[1-methyl-4-(1-methylimidazole-2-amido)pyrrol-2-yl]formamido]propanamido)-N-[1-methyl-5-[(2-[[1-methyl-2-([2-[methyl(3-[methyl[3-(methylamino)propyl]amino]propyl)carbamoyl]ethyl]carbamoyl)imidazol-4-yl]carbamoyl]ethyl)carbamoyl]pyrrol-3-yl]imidazole-2-carboxamide was obtained as yellow oil. LC/MS: mass calcd. For C₄₅H₆₄N₁₈O₈: 984.52, found: 985.40 [M+H]⁺.

### Synthesis of N-(5-[[2-([2-[(2-[[3-(3-aminopropoxy)propyl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide (PA01-TRA(O))

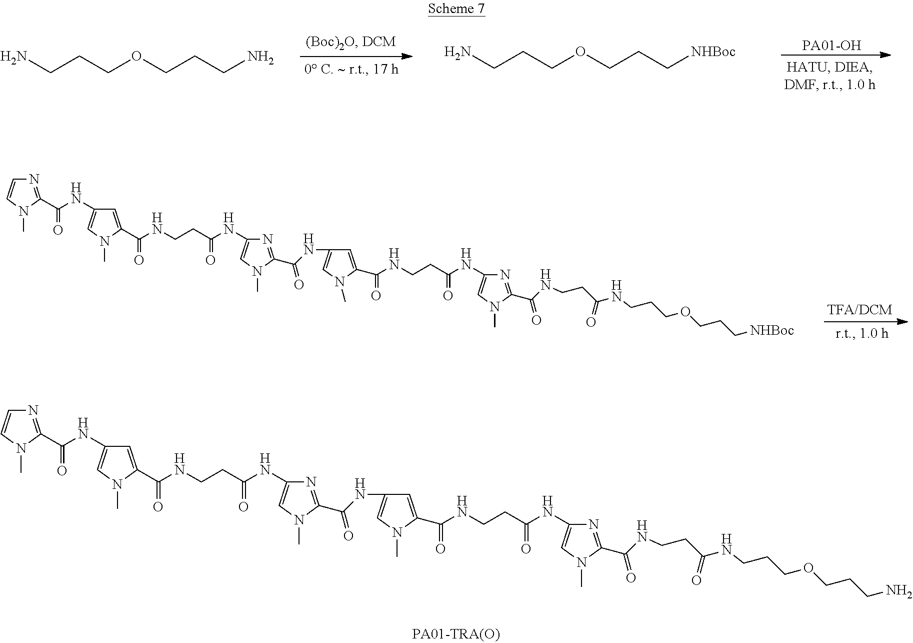 [SMILES: NCCCOCCCN ; CC(C)(C)OC(=O)NCCCOCCCN ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCCOCCCNC(=O)OC(C)(C)C)n1 ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCCOCCCN)n1]

### Step 1: Synthesis of tert-butyl N-[3-(3-aminopropoxy)propyl]carbamate

The procedure was the same as tert-butyl(3-((3-aminopropyl)(methyl)amino)propyl)carbamate. 1.00 g of 3-(3-aminopropoxy)propan-1-amine was used, 430.00 mg of tert-butyl N-[3-(3-aminopropoxy)propyl]carbamate was obtained as yellow oil (24.47% yield). LC/MS: mass calcd. For C₁₁H₂₄N₂O₃:232.18, found: 233.15 [M+H]⁺.

### Step 2: Synthesis of tert-butyl N-(3-[3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido] imidazol-2-yl]formamido)propanamido]propoxy]propyl)carbamate

The procedure was the same as tert-butyl (3-(methyl(3-(3-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamido)propanamido)propyl)amino)propyl)carbamate. 100.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH) was used, 90.00 mg of tert-butyl N-(3-[3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propoxy]propyl)carbamate was obtained as yellow oil (64.37% yield). LC/MS: mass calcd. For C₄₇H₆₅N₁₇O₁₁:1043.50, found: 1044.75 [M+H]⁺.

### Step 3: Synthesis of N-(5-[[2-([2-[(2-[[3-(3-aminopropoxy)propyl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide (PA01-TRA(O))

The procedure was the same as N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA). 90.00 mg of tert-butyl N-(3-[3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propoxy]propyl)carbamate was used, 90.00 mg crude of N-(5-[[2-([2-[(2-[[3-(3-aminopropoxy)propyl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide was obtained as yellow oil. LC/MS: mass calcd. For C₄₂H₅₇N₁₇O₉: 943.45, found: 944.70 [M+H]⁺.

### Synthesis of N-[5-[(2-[[2-([2-[(7-aminoheptyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide (PA01-TRA(CH2))

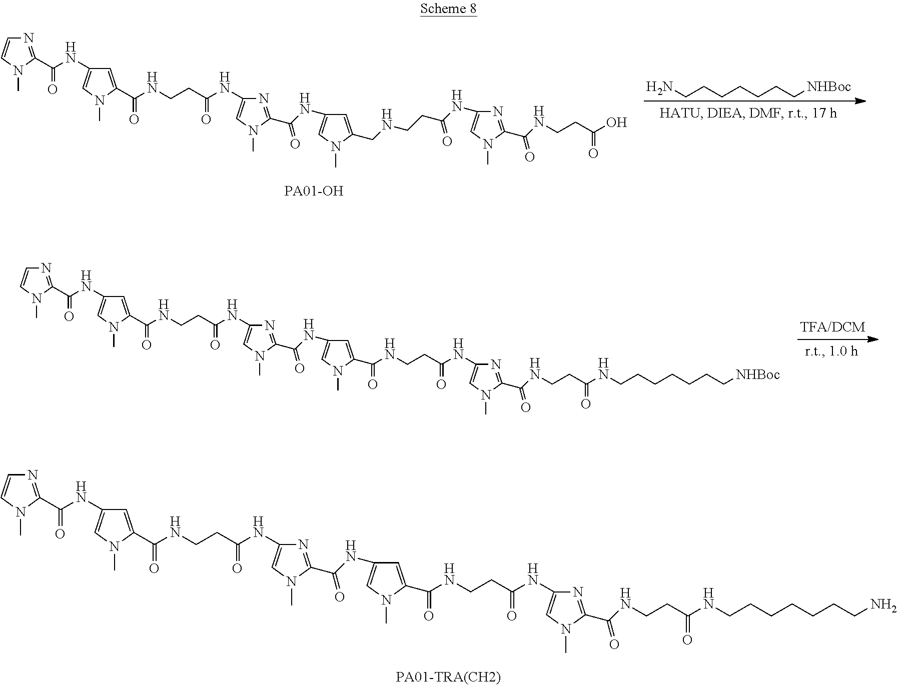 [SMILES: Cn1cc(NC(=O)c2nc(NC(=O)CCNC(=O)c3cc(NC(=O)c4nccn4C)cn3C)cn2C)cc1CNCCC(=O)Nc1cn(C)c(C(=O)NCCC(=O)O)n1 ; CC(C)(C)OC(=O)NCCCCCCCN ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCCCCCCNC(=O)OC(C)(C)C)n1 ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCCCCCCN)n1]

### Step 1: Synthesis of tert-butyl N-[7-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]heptyl]carbamate

The procedure was the same as tert-butyl (3-(methyl(3-(3-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamido)propanamido)propyl)amino)propyl)carbamate. 150.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH) was used, 100.00 mg of tert-butyl N-[7-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]heptyl]carbamate was obtained as yellow oil (33.23% yield). LC/MS: mass calcd. For C₄₈H₆₇N₁₇O₁₀: 1041.53, found: 1042.85 [M+H]⁺.

### Step 2: Synthesis of N-[5-[(2-[[2-([2-[(7-aminoheptyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA). 90.00 mg of tert-butyl N-[7-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]heptyl]carbamate was obtained, 90.00 mg crude of N-[5-[(2-[[2-([2-[(7-aminoheptyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as yellow oil. LC/MS: mass calcd. For C₄₃H₅₉N₁₇O₈: 941.47, found: 942.75 [M+H]⁺.

### Synthesis of N-[5-([2-[(2-[[2-([3-[(3-aminopropyl)(methyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide (PA05-TRA)

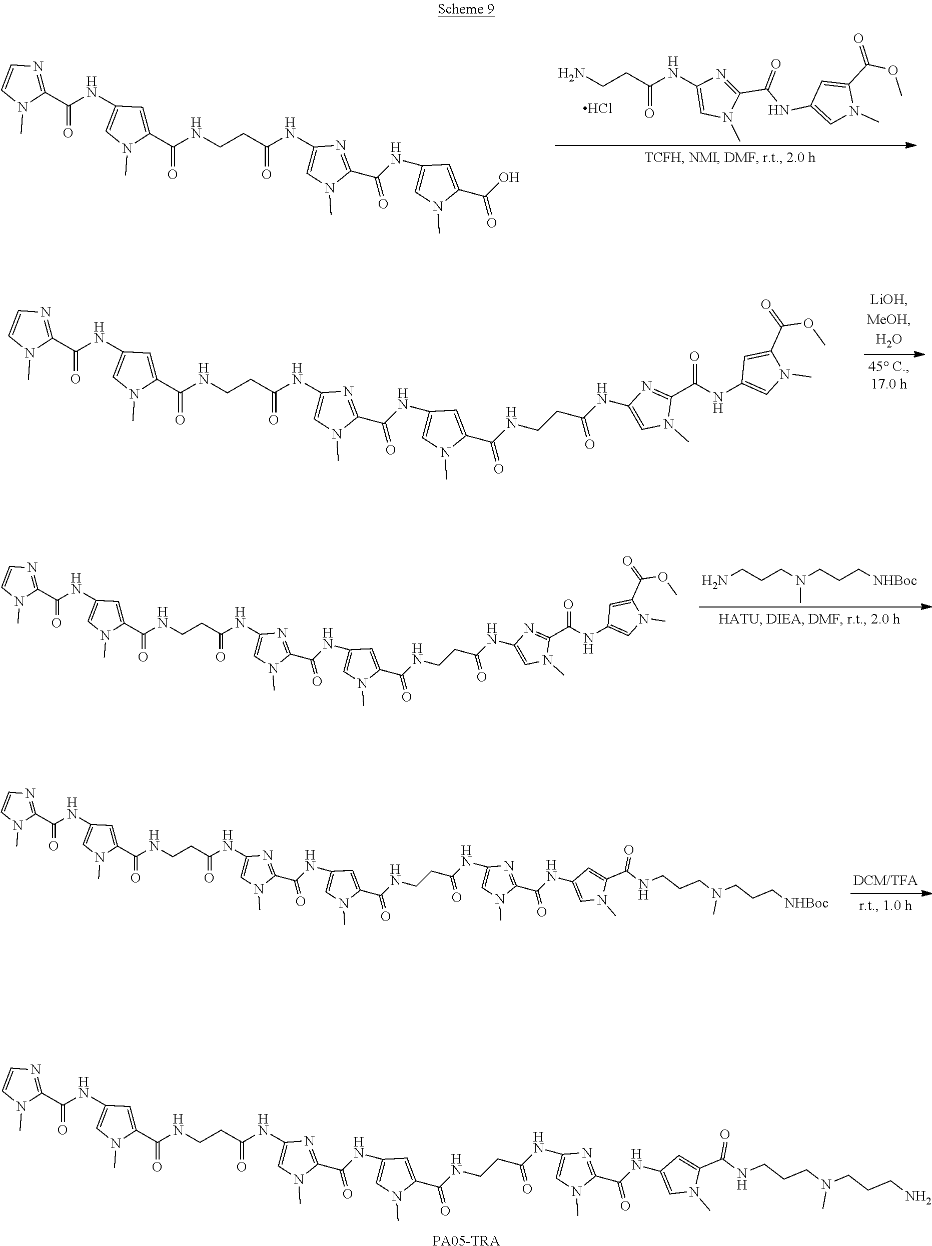

### Step 1: Synthesis of methyl 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido] pyrrole-2-carboxylate

To a solution of 1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxylic acid (1.00 g, 1.77 mmol, 1.00 equiv) in DMF (10.00 mL) was added NMI (727.14 mg, 8.86 mmol, 5.00 equiv), TCFH (546.68 mg, 1.95 mmol, 1.10 equiv) and methyl 4-[4-(3-aminopropanamido)-1-methylimidazole-2-amido]-1-methylpyrrole-2-carboxylate (748.19 mg, 1.95 mmol, 1.10 equiv). Then the reaction was stirred at room temperature for 2 h. The reaction mixture was purified by reverse flash chromatography with the following conditions: column, C18 column; mobile phase, MeOH in H₂O (0.05% TFA), 5% to 75% gradient in 70 min; detector, UV 254 nm. The fractions were combined and concentrated to afford methyl 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido] pyrrol-2-yl]formamido)propanamido]imidazole-2-amido]pyrrole-2-carboxylate (1.00 g, 63.09% yield) as white solid. LC/MS: mass calcd. For C₄₀H₄₆N₁₆O₉: 894.36, found: 895.55 [M+H].

### Step 2: Synthesis of 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrole-2-carboxylic acid

The procedure was the same as 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH). 1.00 g of methyl 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrole-2-carboxylate was used, 800.00 mg of 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrole-2-carboxylic acid was obtained as white solid(81.27% yield). LC/MS: mass calcd. For C₃₉H₄₄N₁₆O₉: 880.35, found: 881.45 [M+H]⁺.

### Step 3: Synthesis of tert-butyl N-[3-[methyl([3-[(1-methyl-4-[1-methyl-4-[3 ([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido) propanamido]imidazole-2-amido]pyrrol-2-yl) formamido]propyl])amino]propyl] carbamate

The procedure was the same as N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide. 800.00 mg of 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrole-2-carboxylic acid was used, 800.00 mg of tert-butyl N-[3-[methyl([3-[(1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrol-2-yl)formamido]propyl])amino]propyl]carbamate was obtained as white solid (79.49% yield). LC/MS: mass calcd. For C₅₁H₆₉N₁₉O₁₀: 1107.55 found: 1108.80 [M+H]⁺.

### Step 4: Synthesis of N-[5-([2-[(2-[[2-([3-[(3-aminopropyl)(methyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA). 800.00 mg of tert-butyl N-[3-[methyl([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl])amino]propyl]carbamate was used, 800.00 mg of N-[5-([2-[(2-[[2-([3-[(3-aminopropyl)(methyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as yellow solid. LC/MS: mass calcd. For C₄₆H₆₁N₁₉O₈: 1007.50 found: 1008.80 [M+H]⁺.

### Synthesis of 3-[(1-Methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrol-2-yl)formamido]propanoic acid (PA14-OH)

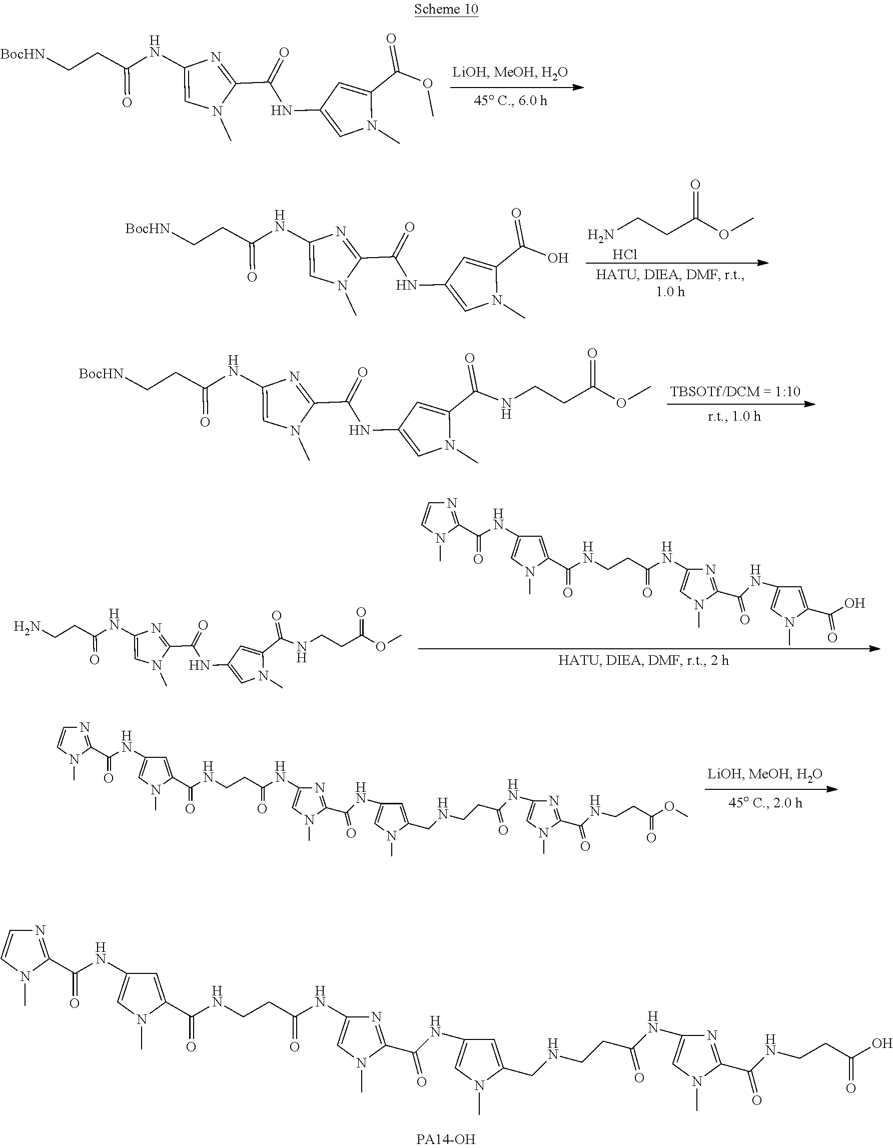

### Step 1: Synthesis of 4-(4-[3-[(Tert-butoxycarbonyl)amino]propanamido]-1-methylimidazole-2-amido)-1-methylpyrrole-2-carboxylic acid

The procedure was the same as 4-[3-[(Tert-butoxycarbonyl)amino]propanamido]-1-methylimidazole-2-carboxylic acid. 2.50 g methyl 4-(4-[3-[(tert-butoxycarbonyl)amino]propanamido]-1-methylimidazole-2-amido)-1-methylpyrrole-2-carboxylate was used, 2.12 g of 4-(4-[3-[(Tert-butoxycarbonyl)amino] propanamido]-1-methylimidazole-2-amido)-1-methylpyrrole-2-carboxylic acid was obtained as white solid (85.94% yield). LC/MS: mass calcd. For C₁₉H₂₆N₆O₆: 434.19, found: 435.20 [M+H]⁺.

### Step 2: Synthesis of methyl 3-[[4-(4-[3-[(tert-butoxycarbonyl)amino] propanamido]-1-methylimidazole-2-amido)-1-methylpyrrol-2-yl]formamido]propanoate

The procedure was the same as methyl 3-[(4-[3-[(tert-butoxycarbonyl)amino]propanamido]-1-methylimidazol-2-yl)formamido]propanoate. 2.12 g of 4-(4-[3-[(tert-butoxycarbonyl)amino]propanamido]-1-methylimidazole-2-amido)-1-methylpyrrole-2-carboxylic acid was used, 2.30 g of methyl 3-[[4-(4-[3-[(tert-butoxycarbonyl)amino]propanamido]-1-methylimidazole-2-amido)-1-methylpyrrol-2-yl]formamido]propanoate was obtained as yellow oil (87.33% yield). LC/MS: mass calcd. For C₂₃H₃₃N₇O₇: 519.24, found: 520.35 [M+H]⁺.

### Step 3: Synthesis of methyl 3-([4-[4-(3-aminopropan-amido)-1-methylimidazole-2-amido]-1-methylpyrrol-2-yl]formamido)propanoate

To a stirred solution of methyl 3-[[4-(4-[3-[(tert-butoxycarbonyl)amino]propanamido]-1-methylimidazole-2-amido)-1-methylpyrrol-2-yl]formamido]propanoate (1.50 g, 2.89 mmol, 1.00 equiv) in DCM (10.00 mL) was added TBSOTf (1.00 mL) dropwise at room temperature. The resulting mixture was stirred for 1 h at room temperature. The resulting mixture was concentrated under vacuum. Methyl 3-([4-[4-(3-aminopropan-amido)-1-methylimidazole-2-amido]-1-methylpyrrol-2-yl]formamido)propanoate (1.70 g, crude) was obtained as red oil. LC/MS: mass calcd. For C₁₈H₂₅N₇O₅: 419.19, found: 420.15 [M+H]⁺.

### Step 4: Synthesis of methyl 3-[(1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrol-2-yl)formamido]propanoate

The procedure was the same as methyl 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoate. 800.00 mg of 1-methyl-4-[1-methyl-4-(3-[[1-methyl-4-(1-methylimidazole-2-amido)pyrrol-2-yl]formamido]propanamido)imidazole-2-amido]pyrrole-2-carboxylic acid was used, 800.00 mg of methyl 3-[(1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrol-2-yl)formamido]propanoate was obtained as yellow solid (41.11% yield). LC/MS: mass calcd. For C₄₂H₄₉N₁₇O₁₀: 965.40, found: 966.40 [M+H]⁺.

### Step 5: Synthesis of 3-[(1-Methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido] pyrrol-2-yl)formamido]propanoic acid

The procedure was the same as 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH). 800.00 mg of methyl 3-[(1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrol-2-yl)formamido]propanoate was used, 650 mg of 3-[(1-Methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrol-2-yl)formamido]propanoic acid was obtained as yellow solid (68.67% yield). LC/MS: mass calcd. For C₄₂H₄₉N₁₇O₁₀: 951.38, found: 952.35 [M+H]⁺.

### Synthesis of 4-(3-[[4-(4-amino-1-methylimidazole-2-amido)-1-methylpyrrol-2-yl]formamido]propanamido)-1-methyl-N-[1-methyl-5-([2-[(1-methyl-2-[[2-(propylcarbamoyl)ethyl]carbamoyl]imidazol-4-yl)carbamoyl]ethyl] carbamoyl)pyrrol-3-yl]imidazole-2-carboxamide (PA15)

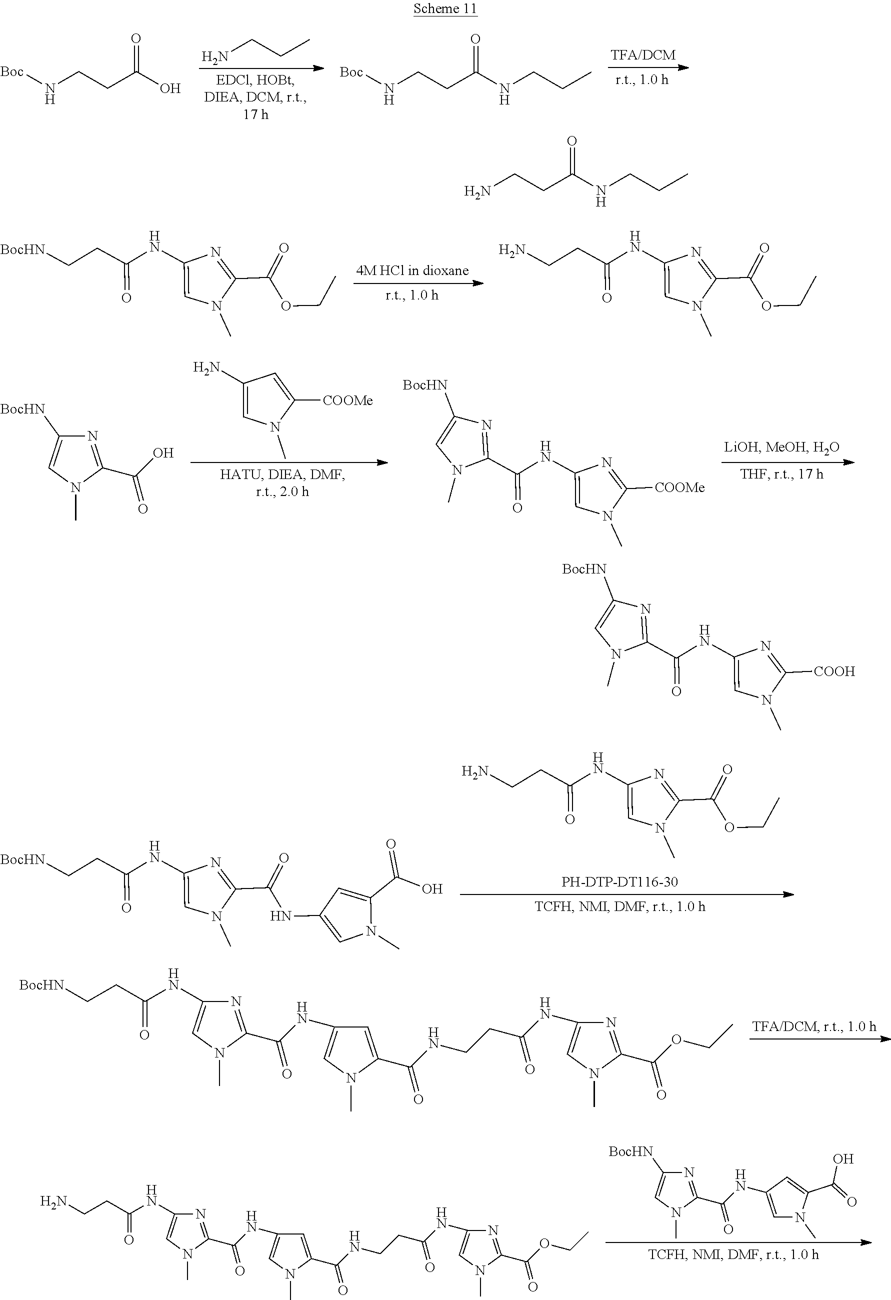

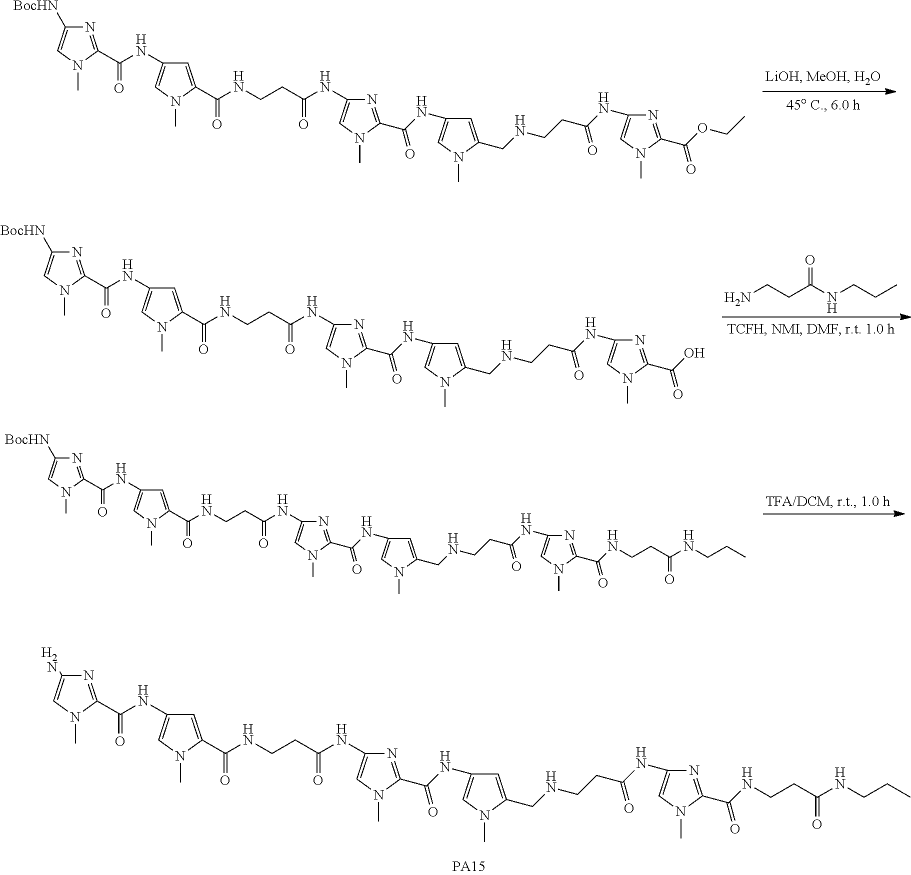

### Step 1: Synthesis of tert-butyl (3-oxo-3-(propylamino)propyl)carbamate

To a stirred solution of 3-[(tert-butoxycarbonyl)amino]propanoic acid (1000.00 mg, 5.29 mmol, 1.00 equiv) in DCM (30.00 mL) was added EDC (114.48 mg, 5.81 mmol, 1.10 equiv), HOBt (785.56 mg, 5.81 mmol, 1.10 equiv) and propylamine (312.41 mg, 5.23 mmol, 1.00 equiv) and DIEA (2732.26 mg, 21.14 mmol, 4.00 equiv) in portions at room temperature. The resulting mixture was stirred for 17 h at room temperature. The reaction was quenched with cool water (30 mL). The resulting mixture was extracted with dichloromethane (3×50 mL). The organic layers were combined, dried over anhydrous sodium sulfate, filtered and concentrated. Tert-butyl (3-oxo-3-(propylamino)propyl)carbamate (600.00 mg, crude) was obtained as white solid. LC/MS: mass calcd. For C₁₁H₂₂N₂O₃: 230.16, found: 231.15 [M+H]⁺.

### Step 2: Synthesis of 3-amino-N-propylpropanamide

To a stirred solution of tert-butyl N-[2-(propylcarbamoyl)ethyl]carbamate (500.00 mg, 2.17 mmol, 1.00 equiv) in DCM (10.00 mL) was added TFA (2.00 mL) dropwise at room temperature. The resulting mixture was stirred for 1 h at room temperature. The resulting mixture was concentrated under vacuum. 3-Amino-N-propylpropanamide (500.00 mg, crude) was obtained as yellow oil. LC/MS: mass calcd. For C₆H₁₄N₂O: 130.11, found: 131.20 [M+H]⁺.

### Step 3: Synthesis of ethyl 4-(3-aminopropanamido)-1-methylimidazole-2-carboxylate

The procedure was the same as methyl 4-[4-(3-aminopropanamido)-1-methylimidazole-2-amido]-1-methylpyrrole-2-carboxylate hydrochloride. 4.00 g of ethyl 4-[3-[(tert-butoxycarbonyl)amino]propanamido]-1-methylimidazole-2-carboxylate was used, 4.00 g crude of ethyl 4-(3-aminopropanamido)-1-methylimidazole-2-carboxylate was obtained as white solid. LC/MS: mass calcd. For C₁₀H₁₆N₄O₃: 240.12, found: 241.15 [M+H]⁺.

### Step 4: Synthesis of methyl 4-(4-((tert-butoxycarbonyl)amino)-1-methyl-1H-imidazole-2-carboxamido)-1-methyl-1H-pyrrole-2-carboxylate

The procedure was the same as ethyl 4-[3-[(tert-butoxycarbonyl)amino]propanamido]-1-methylimidazole-2-carboxylate. 1.00 g of 4-[(tert-butoxycarbonyl)amino]-1-methylimidazole-2-carboxylic acid was used, 1.34 g of methyl 4-(4-((tert-butoxycarbonyl)amino)-1-methyl-1H-imidazole-2-carboxamido)-1-methyl-1H-pyrrole-2-carboxylate was obtained as brown solid (85.00% yield). LC/MS: mass calcd. For C₁₇H₂₃N₅O₅: 377.17, found: 378.25 [M+H]⁺.

### Step 5: Synthesis of 4-(4-((tert-butoxycarbonyl)amino)-1-methyl-1H-imidazole-2-carboxamido)-1-methyl-1H-pyrrole-2-carboxylic acid

The procedure was the same as 4-[3-[(Tert-butoxycarbonyl)amino]propanamido]-1-methylimidazole-2-carboxylic acid. 1.33 g of methyl 4-(4-((tert-butoxycarbonyl)amino)-1-methyl-1H-imidazole-2-carboxamido)-1-methyl-1H-pyrrole-2-carboxylate was used, 946 mg of 4-(4-((tert-butoxycarbonyl)amino)-1-methyl-1H-imidazole-2-carboxamido)-1-methyl-1H-pyrrole-2-carboxylic acid was obtained as white solid (74.00% yield). LC/MS: mass calcd. For C₁₆H₂₁N₅O₅: 363.15, found: 364.25 [M+H]⁺.

### Step 6: Synthesis of Ethyl 4-(3-[[4-(4-[3-[(tert-butoxycarbonyl)amino] propanamido]-1-methylimidazole-2-amido)-1-methylpyrrol-2-yl]formamido] propanamido)-1-methylimidazole-2-carboxylate

To a stirred solution of 4-(4-[3-[(tert-butoxycarbonyl)amino]propanamido]-1-methylimidazole-2-amido)-1-methylpyrrole-2-carboxylic acid (3.60 g, 8.29 mmol, 1.00 equiv) in DMF (50.00 mL) was added NMI (2.04 g, 24.86 mmol, 3.00 equiv), TCFH (3.49 g, 12.43 mmol, 1.50 equiv) and ethyl 4-(3-aminopropanamido)-1-methylimidazole-2-carboxylate (2.19 g, 9.12 mmol, 1.10 equiv) in portions at 0 degrees C. The resulting mixture was stirred for 1 h at room temperature. The reaction was quenched with ice/water (150 mL) at 0 degrees C. The precipitated solids were collected by filtration and washed with H₂O (3×30 mL), dried under vacuum. Ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate (5.00 g, 90.08%) was obtained as yellow solid. LC/MS: mass calcd. For C₂₉H₄₀N₁₀O₈: 656.30, found: 657.50 [M+H]⁺.

### Step 7: Synthesis of Ethyl 4-[3-([4-[4-(3-aminopropanamido)-1-methylimidazole-2-amido]-1-methylpyrrol-2-yl]formamido)propanamido]-1-methylimidazole-2-carboxylate

The procedure was the same as 3-amino-N-propylpropanamide. 4.90 g of ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate was used, 4.90 g crude of ethyl 4-[3-([4-[4-(3-aminopropanamido)-1-methylimidazole-2-amido]-1-methylpyrrol-2-yl]formamido)propanamido]-1-methylimidazole-2-carboxylate was obtained as yellow oil. LC/MS: mass calcd. For C₂₄H₃₂N₁₀O₆: 556.25, found: 557.45 [M+H]⁺.

### Step 8: Synthesis of ethyl 4-(3-[[4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrol-2-yl]formamido]propanamido)-1-methylimidazole-2-carboxylate

The procedure was the same as ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate. 2.80 g of 4-[4-[(tert-butoxycarbonyl)amino]-1-methylimidazole-2-amido]-1-methylpyrrole-2-carboxylic acid was used, 5.00 g of ethyl 4-(3-[[4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrol-2-yl]formamido]propanamido)-1-methylimidazole-2-carboxylate was obtained as yellow solid (71.94% yield). LC/MS: mass calcd. For C₄₀H₅₁N₁₅O₁₀: 901.39, found: 902.70 [M+H]⁺.

### Step 9: 4-(3-[[4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrol-2-yl]formamido]propanamido)-1-methylimidazole-2-carboxylic acid

The procedure was the same as 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH). 2.00 of ethyl 4-(3-[[4-(4-[3-[(4-[4-[(tert-butoxycarbonyl) amino]-1-methylimidazole-2-amido]-1-methylpyrrol-2-yl)formamido]propanamido]-1-methylimidazole-2-amido)-1-methylpyrrol-2-yl]formamido]propanamido)-1-methylimidazole-2-carboxylate was used, 1.60 g of 4-(3-[[4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrol-2-yl]formamido]propanamido)-1-methylimidazole-2-carboxylic acid was obtained as yellow solid (71.60% yield). LC/MS: mass calcd. For C₃₈H₄₇N₁₅O₁₀: 873.36, found: 874.55 [M+H]⁺.

### Step 10: Synthesis of tert-butyl N-(1-methyl-2-[[1-methyl-5-([2-[(1-methyl-2-[[1-methyl-5-([2-[(1-methyl-2-[[2-(propylcarbamoyl)ethyl]carbamoyl]imidazol-4-yl)carbamoyl]ethyl]carbamoyl)pyrrol-3-yl]carbamoyl]imidazol-4-yl)carbamoyl]ethyl]carbamoyl)pyrrol-3-yl]carbamoyl]imidazol-4-yl)carbamate

The procedure was the same as ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate. 550.00 mg of 4-(3-[[4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrol-2-yl]formamido] propanamido)-1-methylimidazole-2-carboxylic acid was used, 310.00 mg of tert-butyl N-(1-methyl-2-[[1-methyl-5-([2-[(1-methyl-2-[[1-methyl-5-([2-[(1-methyl-2-[[2-(propylcarbamoyl)ethyl]carbamoyl]imidazol-4-yl)carbamoyl] ethyl]carbamoyl)pyrrol-3-yl]carbamoyl]imidazol-4-yl)carbamoyl]ethyl]carbamoyl)pyrrol-3-yl]carbamoyl]imidazol-4-yl)carbamate was obtained as yellow solid (44.96% yield). LC/MS: mass calcd. For C₄₄H₅₉N₁₇O₁₀: 985.46, found: 986.35 [M+H]⁺.

### Step 11: Synthesis of 4-(3-[[4-(4-amino-1-methylimidazole-2-amido)-1-methylpyrrol-2-yl]formamido]propanamido)-1-methyl-N-[1-methyl-5-([2-[(1-methyl-2-[[2-(propylcarbamoyl)ethyl]carbamoyl]imidazol-4-yl)carbamoyl]ethyl] carbamoyl)pyrrol-3-yl]imidazole-2-carboxamide (PA15)

The procedure was the same as 3-amino-N-propylpropanamide. 140.00 mg of tert-butyl N-(1-methyl-2-[[1-methyl-5-([2-[(1-methyl-2-[[1-methyl-5-([2-[(1-methyl-2-[[2-(propylcarbamoyl)ethyl]carbamoyl]imidazol-4-yl)carbamoyl]ethyl]carbamoyl)pyrrol-3-yl]carbamoyl]imidazole-4-yl)carbamoyl]ethyl]carbamoyl)pyrrol-3-yl]carbamoyl]imidazol-4-yl)carbamate was used, 140.00 mg crude of 4-(3-[[4-(4-amino-1-methylimidazole-2-amido)-1-methylpyrrol-2-yl]formamido]propanamido)-1-methyl-N-[1-methyl-5-([2-[(1-methyl-2-[[2-(propylcarbamoyl)ethyl]carbamoyl]imidazol-4-yl)carbamoyl]ethyl]carbamoyl)pyrrol-3-yl]imidazole-2-carboxamide was obtained as yellow oil. LC/MS: mass calcd. For C₃₉H₅₁N₁₇O₈: 885.41, found: 886.60 [M+H]⁺.

### Synthesis of 4-[3-[(4-[4-[3-([4-[4-(3-aminopropanamido)-1-methylimidazole-2-amido]-1-methylpyrrol-2-yl]formamido)propanamido]-1-methylimidazole-2-amido]-1-methylpyrrol-2-yl)formamido]propanamido]-1-methyl-N-propylimidazole-2-carboxamide (PA16-CO—NH-n-Pr)

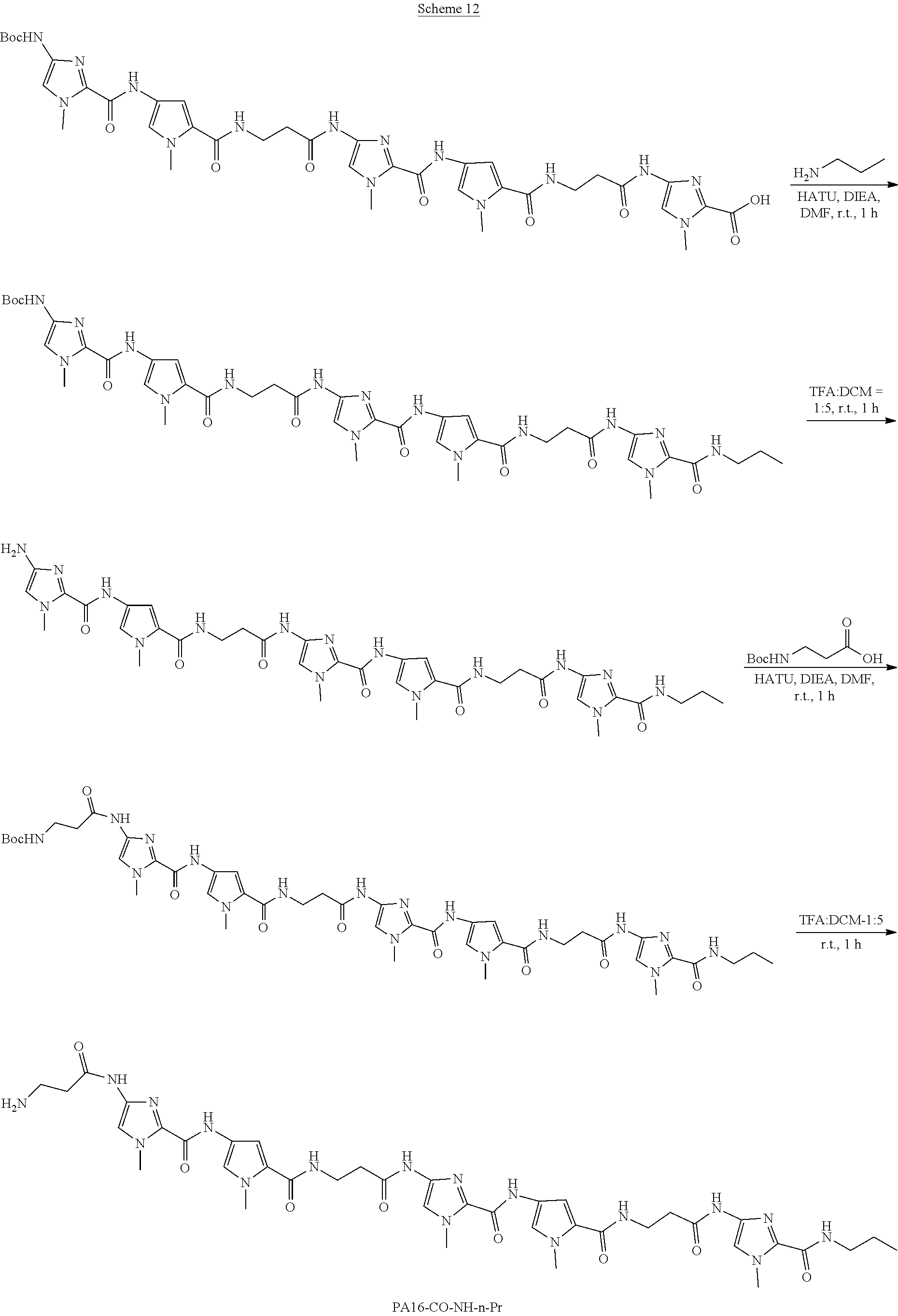

### Step 1: Synthesis of tert-butyl N-[1-methyl-2-([1-methyl-5-[(2-[[1-methyl-2-([1-methyl-5-[(2-[[1-methyl-2-(propylcarbamoyl)imidazol-4-yl]carbamoyl]ethyl) carbamoyl]pyrrol-3-yl]carbamoyl)imidazol-4-yl]carbamoyl]ethyl)carbamoyl]pyrrol-3-yl]carbamoyl) imidazol-4-yl]carbamate

The procedure was the same as ethyl 4-[3-[(tert-butoxycarbonyl)amino] propanamido]-1-methylimidazole-2-carboxylate. 1.00 g of 4-(3-[[4-(4-[3-[(4-[(4-[(tert-butoxycarbonyl)amino]-1-methylimidazole-2-amido]-1-methylpyrrol-2-yl)formamido]propanamido]-1-methylimidazole-2-amido)-1-methylpyrrol-2-yl] formamido]propanamido)-1-methylimidazole-2-carboxylic acid was used, 680.00 mg of tert-butyl N-[1-methyl-2-([1-methyl-5-[(2-[[1-methyl-2-([1-methyl-5-[(2-[[1-methyl-2-(propylcarbamoyl)imidazol-4-yl]carbamoyl]ethyl)carbamoyl]pyrrol-3-yl]carbamoyl)imidazol-4-yl]carbamoyl]ethyl)carbamoyl]pyrrol-3-yl]carbamoyl)imidazol-4-yl]carbamate was obtained as brown solid (57.86% yield). LC/MS: mass calcd. For C₄₁H₅₄N₁₆O₉: 914.43, found: 915.65 [M+H]⁺.

### Step 2: Synthesis of 4-[3-([4-[4-(3-[[4-(4-amino-1-methylimidazole-2-amido)-1-methylpyrrol-2-yl]formamido]propanamido)-1-methylimidazole-2-amido]-1-methylpyrrol-2-yl]formamido)propanamido]-1-methyl-N-propylimidazole-2-carboxamide

The procedure was the same as 3-amino-N-propylpropanamide. 657.00 mg of tert-butyl N-[1-methyl-2-([1-methyl-5-[(2-[[1-methyl-2-([1-methyl-5-[(2-[[1-methyl-2-(propylcarbamoyl)imidazol-4-yl]carbamoyl]ethyl)carbamoyl]pyrrol-3-yl]carbamoyl)imidazol-4-yl]carbamoyl]ethyl)carbamoyl]pyrrol-3-yl]carbamoyl)imidazol-4-yl]carbamate was used, 630.00 mg crude of 4-[3-([4-[4-(3-[[4-(4-amino-1-methylimidazole-2-amido)-1-methylpyrrol-2-yl]formamido]propanamido)-1-methylimidazole-2-amido]-1-methylpyrrol-2-yl] formamido)propanamido]-1-methyl-N-propylimidazole-2-carboxamide was obtained as yellow oil. LC/MS: mass calcd. For C₃₆H₄₆N₁₆O₇: 814.37, found: 815.60 [M+H].

### Step 3: Synthesis of tert-butyl N-(2-[[1-methyl-2-([1-methyl-5-[(2-[[1-methyl-2-([1-methyl-5-[(2-[[1-methyl-2-(propylcarbamoyl)imidazol-4-yl]carbamoyl]ethyl)carbamoyl]pyrrol-3-yl]carbamoyl)imidazol-4-yl]carbamoyl]ethyl)carbamoyl]pyrrol-3-yl]carbamoyl)imidazol-4-yl]carbamoyl]ethyl)carbamate

The procedure was the same as ethyl 4-[3-[(tert-butoxycarbonyl)amino]propanamido]-1-methylimidazole-2-carboxylate, but the product was purified by reverse phase column under NH₃.H₂O system. 630.00 mg of 4-[3-([4-[4-(3-[[4-(4-amino-1-methylimidazole-2-amido)-1-methylpyrrol-2-yl]formamido]propanamido)-1-methylimidazole-2-amido]-1-methylpyrrol-2-yl]formamido)propanamido]-N-ethyl-1-methylimidazole-2-carboxamide was used, 200.00 mg of tert-butyl N-(2-[[1-methyl-2-([1-methyl-5-[(2-[[1-methyl-2-([1-methyl-5-[(2-[[1-methyl-2-(propylcarbamoyl)imidazol-4-yl]carbamoyl]ethyl)carbamoyl]pyrrol-3-yl] carbamoyl)imidazol-4-yl]carbamoyl]ethyl)carbamoyl]pyrrol-3-yl]carbamoyl)imidazol-4-yl]carbamoyl]ethyl)carbamate was obtained as yellow solid (25.80% yield). LC/MS: mass calcd. For C₄₄H₅₉N₁₇O₁₀: 985.46, found: 986.70 [M+H]⁺.

### Step 4: Synthesis of 4-[3-[(4-[4-[3-([4-[4-(3-aminopropanamido)-1-methylimidazole-2-amido]-1-methylpyrrol-2-yl]formamido)propanamido]-1-methylimidazole-2-amido]-1-methylpyrrol-2-yl)formamido]propanamido]-1-methyl-N-propylimidazole-2-carboxamide (PA16-CO—NH-n-Pr)

The procedure was the same as 3-amino-N-propylpropanamide. 100.00 mg of tert-butyl N-(2-[[1-methyl-2-([1-methyl-5-[(2-[[1-methyl-2-([1-methyl-5-[(2-[[1-methyl-2-(propylcarbamoyl)imidazol-4-yl]carbamoyl]ethyl)carbamoyl]pyrrol-3-yl]carbamoyl)imidazol-4-yl]carbamoyl]ethyl)carbamoyl]pyrrol-3-yl]carbamoyl)imidazol-4-yl]carbamoyl]ethyl)carbamate was used, 100.00 mg crude of 4-[3-[(4-[4-[3-([4-[4-(3-aminopropanamido)-1-methylimidazole-2-amido]-1-methylpyrrol-2-yl]formamido)propanamido]-1-methylimidazole-2-amido]-1-methylpyrrol-2-yl)formamido]propanamido]-1-methyl-N-propylimidazole-2-carboxamide was obtained as yellow oil. LC/MS: mass calcd. For C₃₉H₅₁N₁₇O₈: 885.41, found: 886.70 [M+H]⁺.

### Synthesis of 3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanoic acid (PA17-OH)

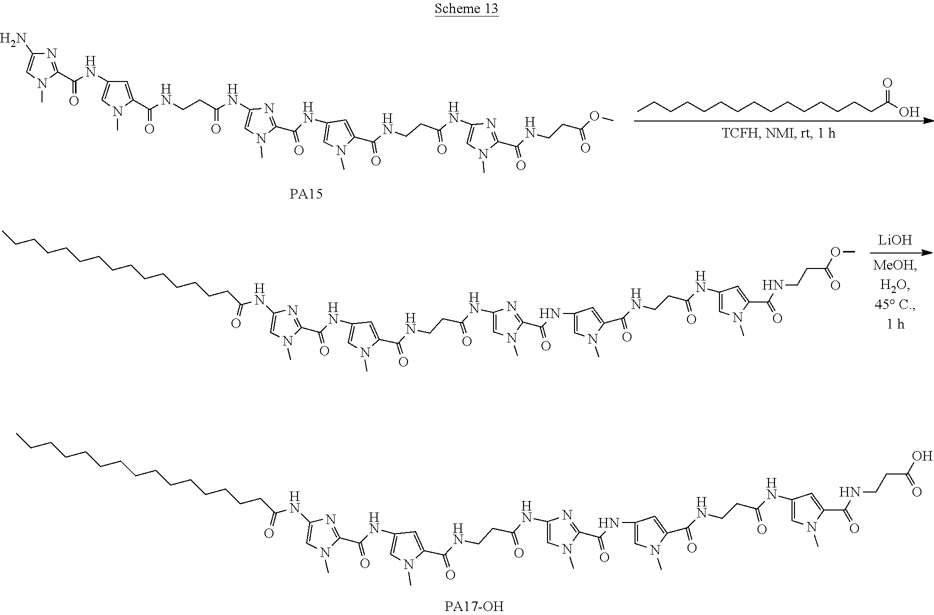

### Step 1: Synthesis of methyl 3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanoate

The procedure was the same as ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate. 500.00 mg of methyl 3-([4-[3-([4-[4-(3-[[4-(4-amino-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanoate was used, 510.00 mg of methyl 3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanoate was obtained as brown solid (79.84% yield). LC/MS: mass calcd. For C₅₃H₇₆N₁₆O₁₀: 1096.59, found: 1097.50 [M+H]⁺.

### Step 2: Synthesis of 3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanoic acid

The procedure was the same as 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH). 270.00 mg of methyl 3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanoate was used, 260.00 mg crude of 3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanoic acid was obtained as brown solid. LC/MS: mass calcd. For C₅₂H₇₄N₁₆O₁₀: 1082.57, found: 1083.90 [M+H]⁺.

### Synthesis of N-[5-([2-[(2-[[5-([2-[(2-[[2-([3-[(3-aminopropyl)(methyl)amino]propyl]carbamoyl)ethyl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]-4-hexadecanamido-1-methylimidazole-2-carboxamide (PA17-TRA)

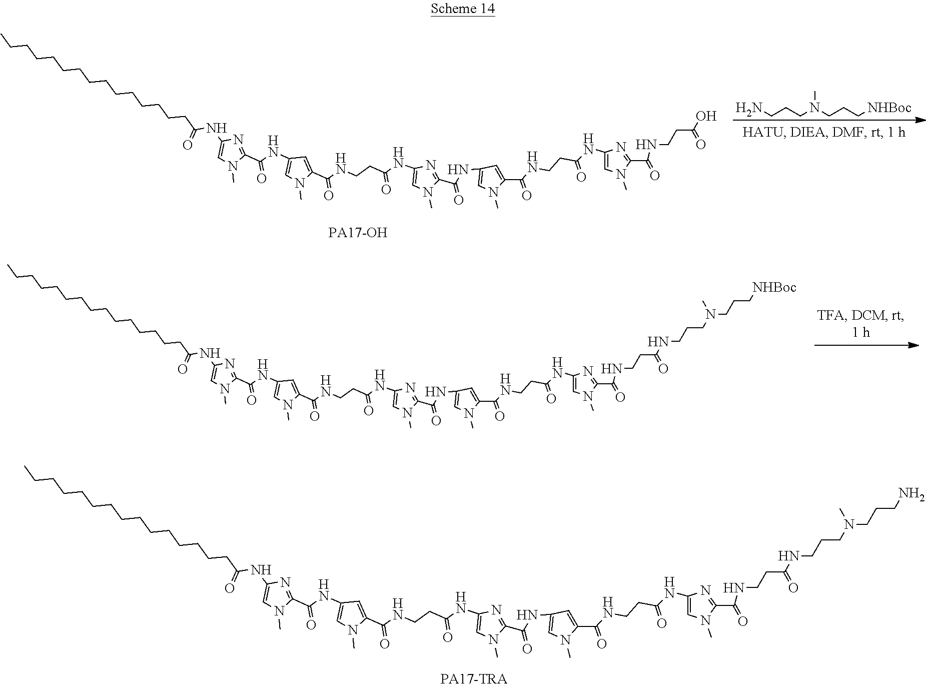

### Step 1: Synthesis of tert-butyl N-[3-([3-[3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanamido]propyl](methyl)amino)propyl]carbamate

The procedure was the same as tert-butyl (3-(methyl(3-(3-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamido)propanamido)propyl)amino)propyl)carbamate. 70.00 mg of 3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanoic acid was used, 50.00 mg of tert-butyl N-[3-([3-[3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanamido]propyl](methyl)amino)propyl]carbamate was obtained as yellow solid (59.04% yield). LC/MS: mass calcd. For C₆₄H₉₉N₁₉O₁₁: 1309.78, found: 1311.20 [M+H]⁺.

### Step 2: Synthesis of N-[5-([2-[(2-[[5-([2-[(2-[[2-([3-[(3-aminopropyl)(methyl) amino]propyl]carbamoyl)ethyl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]-4-hexadecanamido-1-methylimidazole-2-carboxamide (PA17-TRA)

The procedure was the same as N-(3-((3-((3-aminopropyl)(methyl)amino)propyl) amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA). 50.00 mg of tert-butyl N-[3-([3-[3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanamido]propyl](methyl)amino)propyl]carbamate was used, 40.00 mg crude of N-[5-([2-[(2-[[5-([2-[(2-[[2-([3-[(3-aminopropyl)(methyl)amino]propyl]carbamoyl)ethyl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]-4-hexadecanamido-1-methylimidazole-2-carboxamide was obtained as yellow oil. LCMS: mass calcd. For C₅₉H₉₁N₁₉O₉: 1209.72, found: 1211.05 [M+H]⁺.

### Synthesis of 4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrole-2-carboxylic acid (PA18-OH)

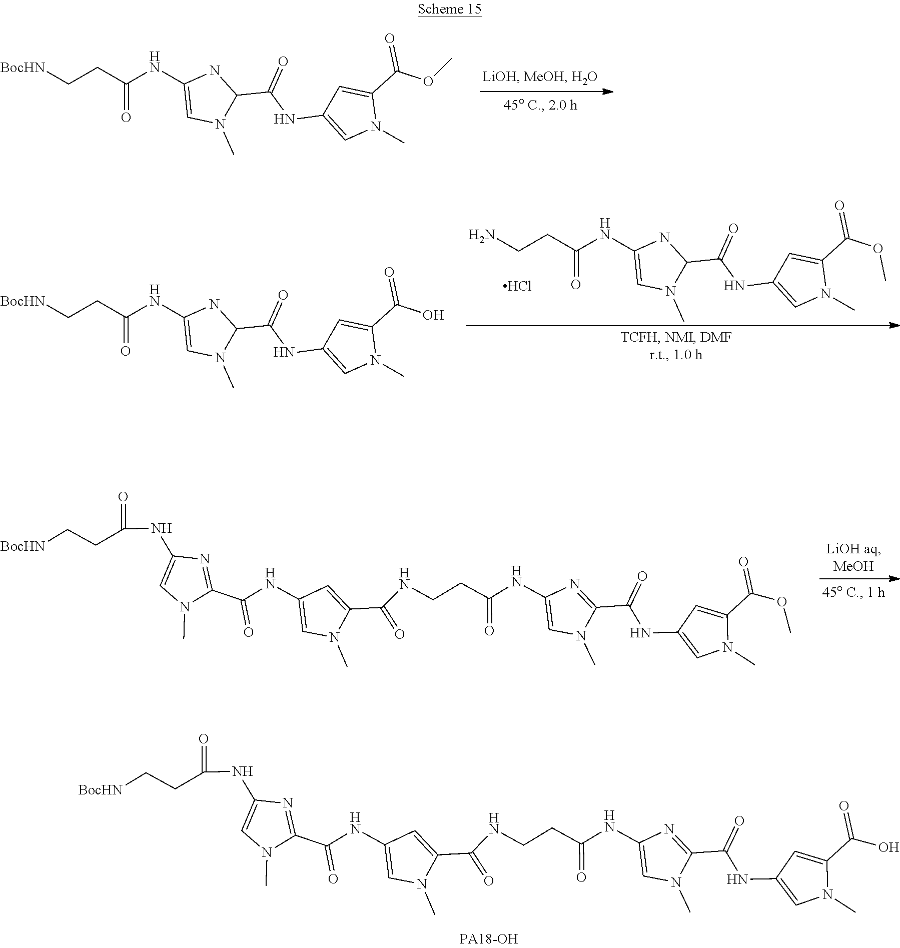

### Step 1: Synthesis of 4-(4-[3-[(tert-butoxycarbonyl)amino]propanamido]-1-methylimidazole-2-amido)-1-methylpyrrole-2-carboxylic acid

The procedure was the same as 4-[3-[(tert-butoxycarbonyl)amino]propanamido]-1-methylimidazole-2-carboxylic acid. 10.00 g of methyl 4-(4-[3-[(tert-butoxycarbonyl)amino]propanamido]-1-methylimidazole-2-amido)-1-methylpyrrole-2-carboxylate was used, 8.50 g of 4-(4-[3[(tert-butoxycarbonyl)amino]propanamido]-1-methylimidazole-2-amido)-1-methylpyrrole-2-carboxylic acid was obtained as white solid (87.74% yield). LC/MS: mass calcd. for C₁₉H₂₆N₆O₆: 434.19, found: 435.20 [M+H]⁺.

### Step 2: Synthesis of Methyl 4-[4-(3-[[4-(4-[3-[(tert-butoxycarbonyl)amino] propanamido]-1-methylimidazole-2-amido)-1-methylpyrrol-2-yl]formamido]propanamido)-1-methylimidazole-2-amido]-1-methylpyrrole-2-carboxylate

The procedure was the same as methyl 4-(4-[3-[(tert-butoxycarbonyl)amino] propanamido]-1-methylimidazole-2-amido)-1-methylpyrrole-2-carboxylate. 1.00 g of 4-(4-[3-[(tert-butoxycarbonyl)amino]propanamido]-1-methylimidazole-2-amido)-1-methylpyrrole-2-carboxylic acid was used, 1.65 g of methyl 4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrol-2-carboxylate was obtained as white solid (80.11% yield) LCMS: mass calcd. for C₃₄H₄₄N₁₂O₉: 764.34, found: 765.50 [M+H]⁺.

### Step 3: Synthesis of 4-[4-(3-[[4-(4-[3-[(tert-butoxycarbonyl)amino] propanamido]-1-methylimidazole-2-amido)-1-methylpyrrol-2-yl]formamido]propanamido)-1-methylimidazole-2-amido]-1-methylpyrrole-2-carboxylic acid

The procedure was the same as 4-[3-[(tert-butoxycarbonyl)amino]propanamido]-1-methylimidazole-2-carboxylic acid. 100.00 mg of 4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrole-2-carboxylate was used, 80.00 mg of 4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrole-2-carboxylic acid was obtained as yellow solid (77.42% yield). LC/MS: mass calcd. for C₃₃H₄₂N₁₂O₉: 750.32, found: 751.30 [M+H]⁺.

### Synthesis of Methyl 4-[4-[3-([4-[4-(3-aminopropanamido)-1-methylimidazole-2-amido]-1-methylpyrrol-2-yl]formamido)propanamido]-1-methylimidazole-2-amido]-1-methylpyrrole-2-carboxylate (PA18-NH2)

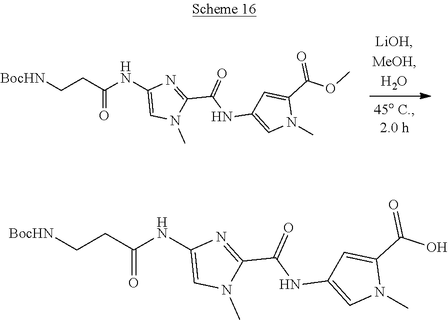 [SMILES: COC(=O)c1cc(NC(=O)c2nc(NC(=O)CCNC(=O)OC(C)(C)C)cn2C)cn1C ; Cn1cc(NC(=O)c2nc(NC(=O)CCNC(=O)OC(C)(C)C)cn2C)cc1C(=O)O]

The procedure was the same as 3-amino-N-propylpropanamide, but the reaction time was 2.0 h. 500.00 mg of methyl 4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrole-2-carboxylate was used, 500.00 mg of methyl 4-[4-[3-([4-[4-(3-aminopropanamido)-1-methylimidazole-2-amido]-1-methylpyrrol-2-yl]formamido)propanamido]-1-methylimidazole-2-amido]-1-methylpyrrole-2-carboxylate was obtained as yellow solid. HRMS: mass calcd. for C₂₉H₃₆N₁₂O₇: 664.2830, found: 665.2891 [M+H]⁺.

### Synthesis of 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylic acid (PA19-OH)

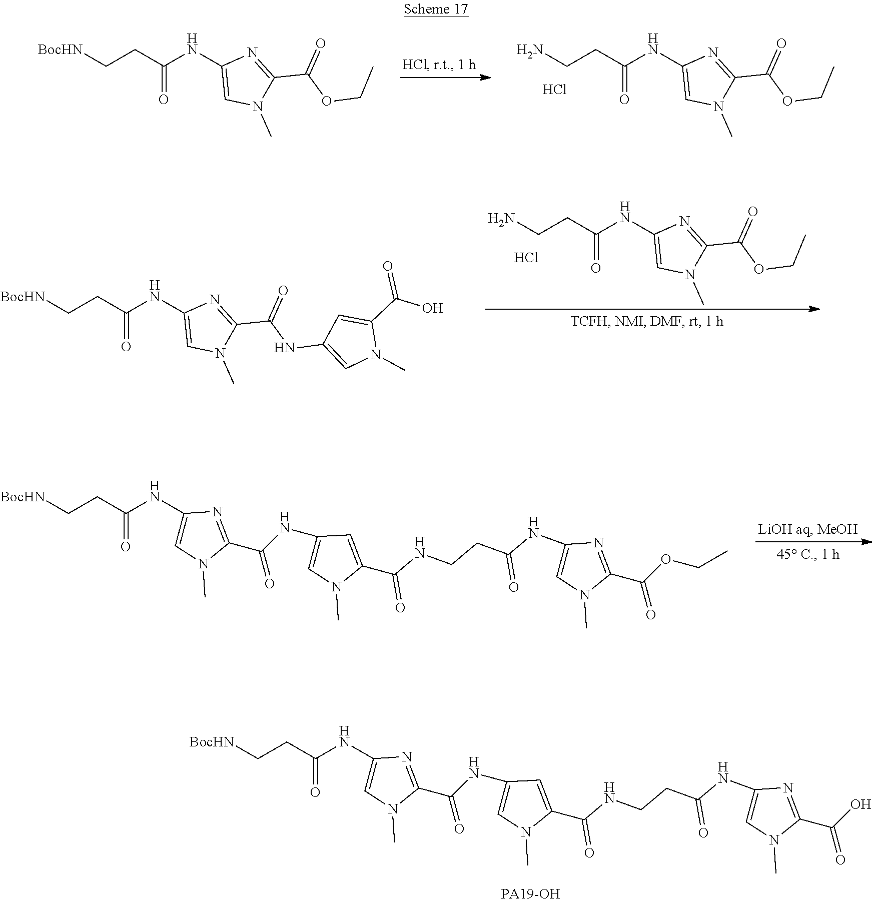

### Step 1: Synthesis of ethyl 4-(3-aminopropanamido)-1-methylimidazole-2-carboxylate

The procedure was the same as methyl 4-[4-(3-aminopropanamido)-1-methylimidazole-2-amido]-1-methylpyrrole-2-carboxylate hydrochloride. 2.00 g of ethyl 4-[3-[(tert-butoxycarbonyl)amino]propanamido]-1-methylimidazole-2-carboxylate was used, 2.00 g crude of ethyl 4-(3-aminopropanamido)-1-methylimidazole-2-carboxylate was obtained as off-white solid. LCMS: mass calcd. For C₁₀H₁₆N₄O₃: 240.12, found: 241.10 [M+H]⁺.

### Step 2: Synthesis of ethyl 4-(3-[[4-(4-[3-[(tert-butoxycarbonyl)amino] propanamido]-1-methylimidazole-2-amido)-1-methylpyrrol-2-yl]formamido]propanamido)-1-methylimidazole-2-carboxylate

The procedure was the same as ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate. 900.00 mg of 4-(4-[3-[(tert-butoxycarbonyl)amino]propanamido]-1-methylimidazole-2-amido)-1-methylpyrrole-2-carboxylic acid was used, 1.10 g of ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate was obtained as off-white solid (75.80% yield). LCMS: mass calcd. For C₂₉H₄₀N₁₀O₈: 656.30, found: 657.50 [M+H]⁺.

### Step 3: Synthesis of 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylic acid

The procedure was the same as 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH). 600 mg of ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate was used, 500.00 mg of 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylic acid was obtained as yellow solid (78.35% yield). LCMS: mass calcd. For C₂₇H₃₆N₁₀O₈: 628.27, found: 629.45 [M+H]⁺.

### Synthesis of Ethyl 4-(3-(4-(4-(3-aminopropanamido)-1-methyl-1H-imidazole-2-carboxamido)-1-methyl-1H-pyrrole-2-carboxamido)propanamido)-1-methyl-1H-imidazole-2-carboxylate (PA19-NH2)

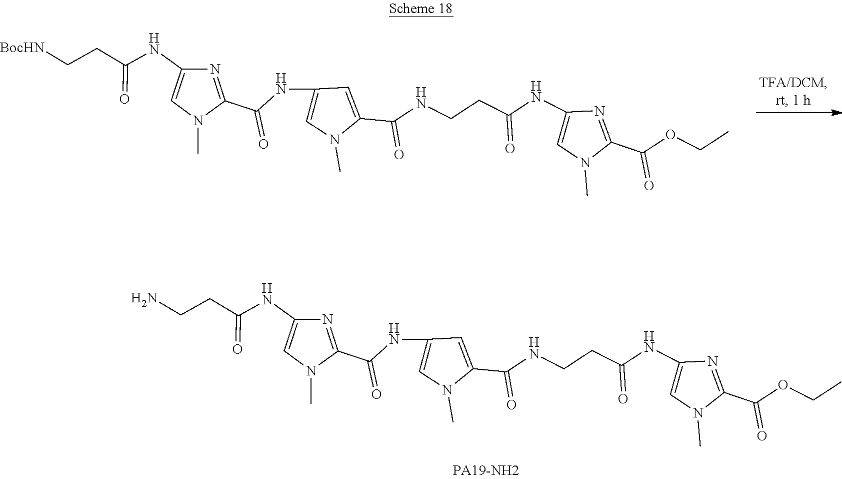 [SMILES: CCOC(=O)c1nc(NC(=O)CCNC(=O)c2cc(NC(=O)c3nc(NC(=O)CCNC(=O)OC(C)(C)C)cn3C)cn2C)cn1C ; CCOC(=O)c1nc(NC(=O)CCNC(=O)c2cc(NC(=O)c3nc(NC(=O)CCN)cn3C)cn2C)cn1C]

The procedure was the same as 3-amino-N-propylpropanamide. 900.00 mg of ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate was used, 900.00 mg crude of ethyl 4-[3-([4-[4-(3-aminopropanamido)-1-methylimidazole-2-amido]-1-methylpyrrol-2-yl]formamido)propanamido]-1-methylimidazole-2-carboxylate was obtained as white solid. LCMS: mass calcd. For C₂₄H₃₂N₁₀O₆: 556.25, found: 557.50 [M+H]⁺.

### Synthesis of 4-(3-(4-(4-(3-(4-(4-(3-((tert-butoxycarbonyl)amino)propanamido)-1-methyl-1H-imidazole-2-carboxamido)-1-methyl-1H-pyrrole-2-carboxamido)propanamido)-1-methyl-1H-imidazole-2-carboxamido)-1-methyl-1H-pyrrole-2-carboxamido)propanamido)-1-methyl-1H-imidazole-2-carboxylic acid (PA16-OH)

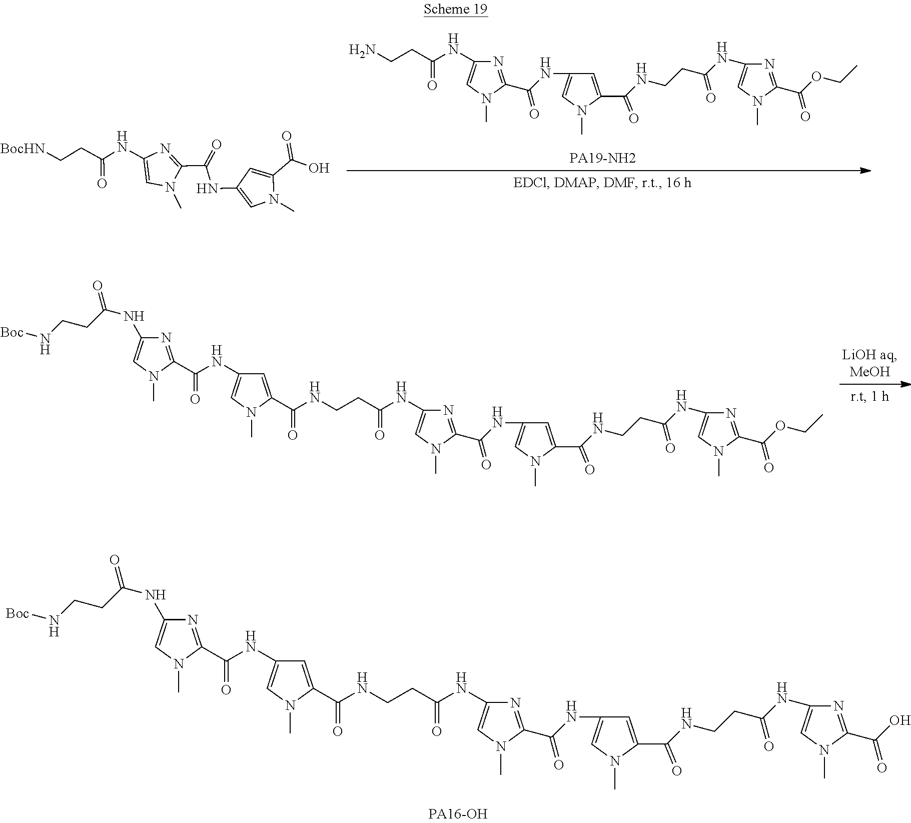 [SMILES: CCOC(=O)c1nc(NC(=O)CCNC(=O)c2cc(NC(=O)c3nc(NC(=O)CCN)cn3C)cn2C)cn1C ; Cn1cc(NC(=O)c2nc(NC(=O)CCNC(=O)OC(C)(C)C)cn2C)cc1C(=O)O ; CCOC(=O)c1nc(NC(=O)CCNC(=O)c2cc(NC(=O)c3nc(NC(=O)CCNC(=O)c4cc(NC(=O)c5nc(NC(=O)CCNC(=O)OC(C)(C)C)cn5C)cn4C)cn3C)cn2C)cn1C ; Cn1cc(NC(=O)c2nc(NC(=O)CCNC(=O)c3cc(NC(=O)c4nc(NC(=O)CCNC(=O)OC(C)(C)C)cn4C)cn3C)cn2C)cc1C(=O)NCCC(=O)Nc1cn(C)c(C(=O)O)n1]

### Step 1: Synthesis of ethyl 4-[3-([4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrol-2-yl]formamido)propanamido]-1-methylimidazole-2-carboxylate

To a stirred solution of 4-(4-[3-[(tert-butoxycarbonyl)amino]propanamido]-1-methylimidazole-2-amido)-1-methylpyrrole-2-carboxylic acid (120.00 mg, 0.276 mmol, 1.00 equiv) and EDCI (134.00 mg, 0.70 mmol, 2.53 equiv), DMAP (86.00 mg, 0.70 mmol, 2.55 equiv) in DMF (5.00 mL) was added ethyl 4-[3-([4-[4-(3-aminopropanamido)-1-methylimidazole-2-amido]-1-methylpyrrol-2-yl]formamido)propanamido]-1-methylimidazole-2-carboxylate (199.85 mg, 0.36 mmol, 1.30 equiv). The resulting solution was stirred for 16 h at room temperature. The resulting mixture was pour into ice/water (20 mL), the precipitated solids were collected by filtration and washed with water (3×10 mL). The solid was dried under reduced pressure to afford the ethyl 4-[3-([4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrol-2-yl]formamido)propanamido]-1-methylimidazole-2-carboxylate (240.00 mg, 80.37%) as yellow solid. LCMS: mass calcd. For C₄₃H₅₆N₁₆O₁₁: 972.43, found: 973.45 [M+H]⁺.

### Step 2: Synthesis of 4-[3-([4-[4-(3-[[4-(4-[3-[(tert-butoxycarbonyl)amino] propanamido]-1-methylimidazole-2-amido)-1-methylpyrrol-2-yl]formamido]propanamido)-1-methylimidazole-2-amido]-1-methylpyrrol-2-yl]formamido)propanamido]-1-methylimidazole-2-carboxylic acid

The procedure was the same with 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH). 400.00 mg of ethyl 4-[3-([4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrol-2-yl]formamido)propanamido]-1-methylimidazole-2-carboxylate was used, 260.00 mg of 4-[3-([4-[4-(3-[[4-(4-[3-[(tert-butoxycarbonyl) amino]propanamido]-1-methylimidazole-2-amido)-1-methylpyrrol-2-yl]formamido]propanamido)-1-methylimidazole-2-amido]-1-methylpyrrol-2-yl]formamido)propanamido]-1-methylimidazole-2-carboxylic acid was obtained as white solid (66.93% yield). LC/MS: mass calcd. For C₄₁H₅₂N₁₆O₁₁: 944.40, found: 945.40[M+H]⁺.

### Synthesis of 4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrole-2-carboxylic acid (PA20-OH)

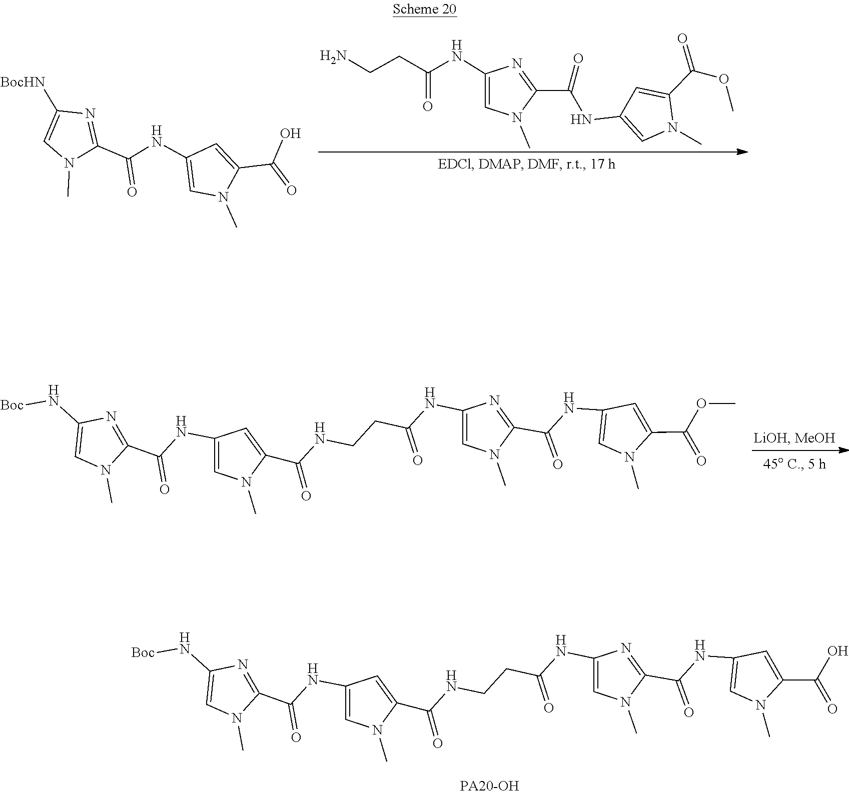 [SMILES: Cn1cc(NC(=O)c2nc(NC(=O)OC(C)(C)C)cn2C)cc1C(=O)O ; COC(=O)c1cc(NC(=O)c2nc(NC(=O)CCN)cn2C)cn1C ; COC(=O)c1cc(NC(=O)c2nc(NC(=O)CCNC(=O)c3cc(NC(=O)c4nc(NC(=O)OC(C)(C)C)cn4C)cn3C)cn2C)cn1C ; Cn1cc(NC(=O)c2nc(NC(=O)CCNC(=O)c3cc(NC(=O)c4nc(NC(=O)OC(C)(C)C)cn4C)cn3C)cn2C)cc1C(=O)O]

### Step 1: Synthesis of 4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrole-2-carboxylate

The procedure was the same as ethyl 4-[3-([4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrol-2-yl]formamido)propanamido]-1-methylimidazole-2-carboxylate. 1.00 g of 4-[4-[(tert-butoxycarbonyl)amino]-1-methylimidazole-2-amido]-1-methylpyrrole-2-carboxylic acid was used, 1.10 g of methyl 4-(4-[3-[(4-[4-[(tert-butoxycarbonyl) amino]-1-methylimidazole-2-amido]-1-methylpyrrol-2-yl)formamido]propanamido]-1-methylimidazole-2-amido)-1-methylpyrrole-2-carboxylate was obtained as dark yellow solid (51.28% yield). LC/MS: mass calcd. For C₃₁H₃₉N₁₁O₈: 693.30, found: 694.15 [M+H]⁺.

### Step 2: Synthesis of 4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrole-2-carboxylic acid

The procedure was the same as 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH). 500.00 mg of methyl 4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrole-2-carboxylate was used, 400.00 mg of 4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrole-2-carboxylic acid was obtained as dark yellow solid (81.65% yield). LCMS: mass calcd. For C₃₀H₃₇N₁₁O₈: 679.28, found: 680.25 [M+H]⁺.

### Example 1B Synthesis of the Protein Binders (Second Binding Domain)

### Synthesis of (S)-2-(6-(4-chlorophenyl)-8-methoxy-1-methyl-4H-benzo[f][1,2,4]triazolo[4,3-a][1,4]diazepin-4-yl)-N-(4-hydroxyphenyl)acetamide (SM10) and (R)-2-(6-(4-chlorophenyl)-8-methoxy-1-methyl-4H-benzo[f][1,2,4]triazolo[4,3-a][1,4]diazepin-4-yl)-N-(4-hydroxyphenyl)acetamide (SM20)

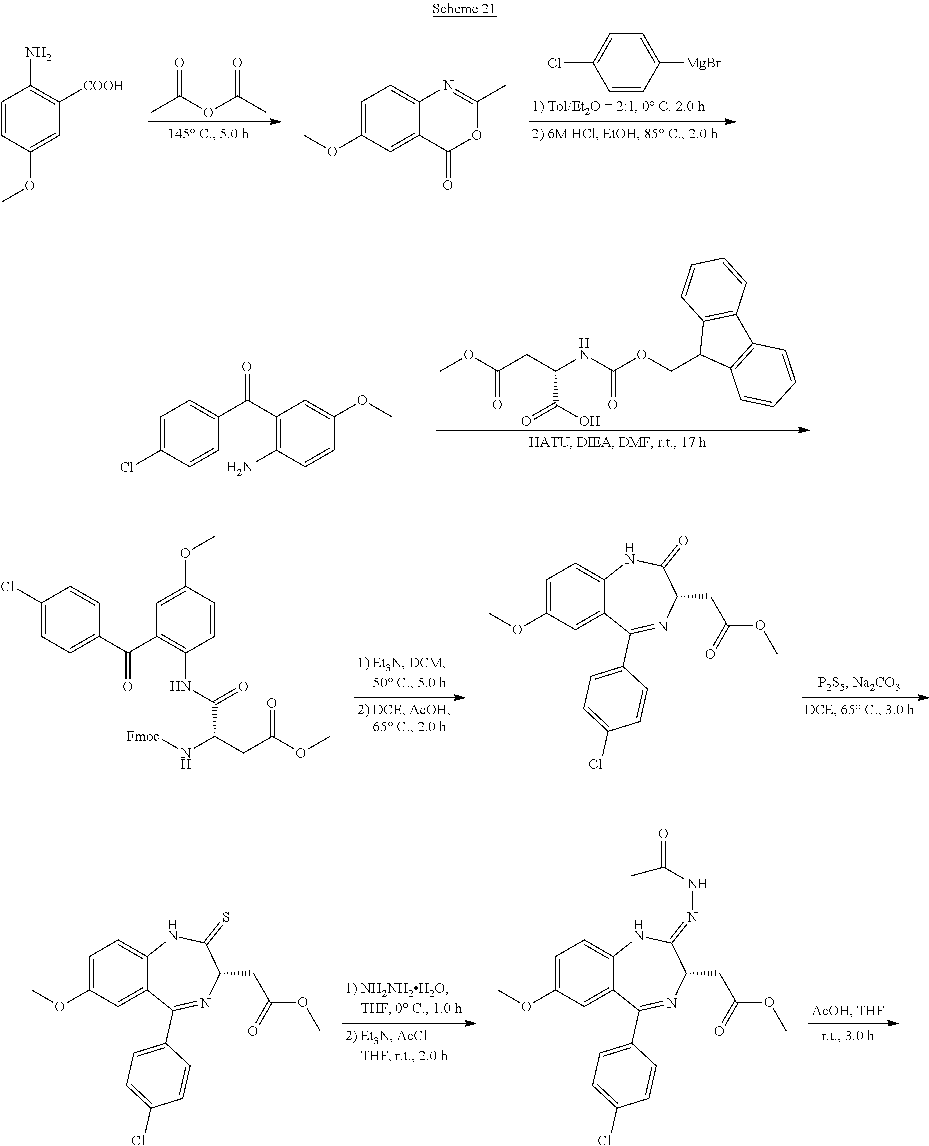

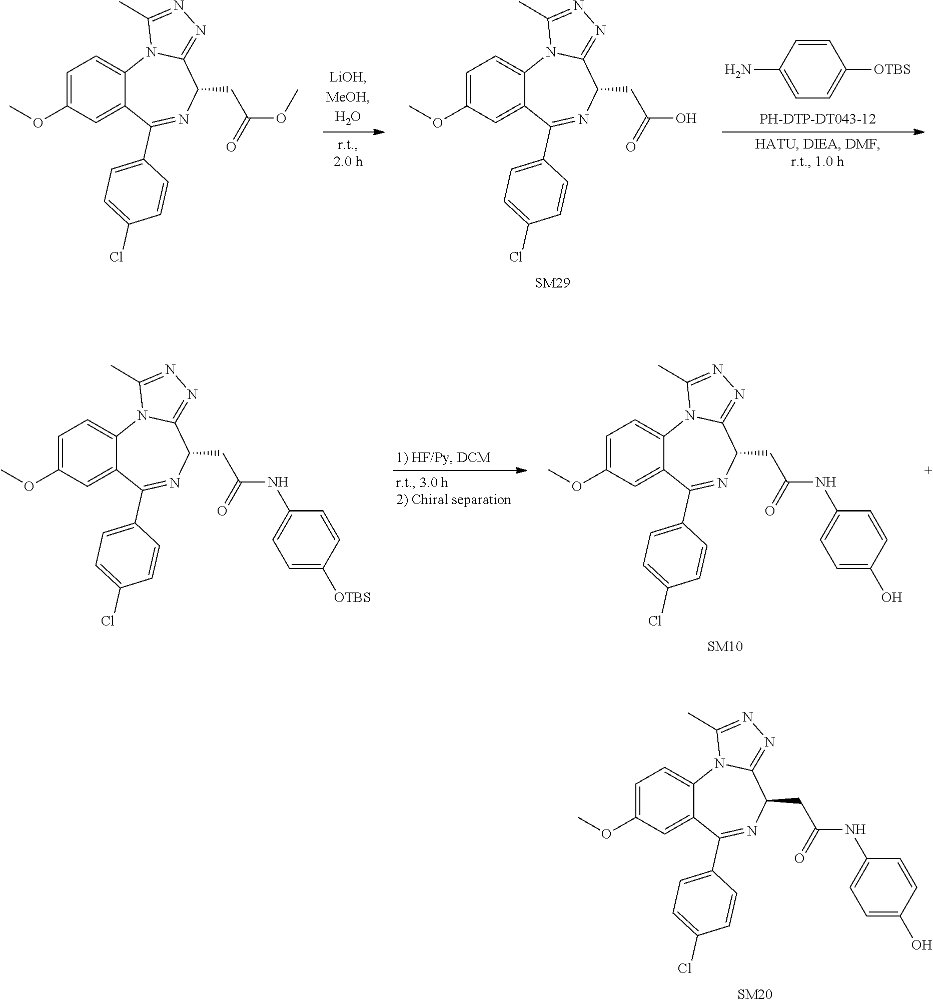

### Step 1: Synthesis of 6-methoxy-2-methyl-4H-benzo [d][1,3]oxazin-4-one

To a 1000 mL round flask was added 2-amino-5-methoxybenzoic acid (55.00 g, 0.33 mol, 1.00 equiv) and acetic anhydride (700.00 mL). The mixture was stirred at 145 degrees C. for 5.0 h. Then the solvent was removed, the residue was washed with Et₂O (100 mL). The solid was filtered out and dried. This resulted 57.00 g (90% yield) of 6-methoxy-2-methyl-4H-benzo[d][1,3]oxazin-4-one as light yellow solid. LC/MS: mass calcd. For C₁₀H₉NO₃: 191.06, found: 192.20 [M+H]⁺.

### Step 2: Synthesis of (2-amino-5-methoxyphenyl)(4-chlorophenyl)methanone

To a solution of 6-methoxy-2-methyl-3,1-benzoxazin-4-one (30.00 g, 156.92 mmol, 1.00 equiv) in Tol (300.00 mL) and Et₂O (150.00 mL) was added bromo (4-chlorophenyl)magnesium (1.0 M in THF, 141.00 mL, 141.00 mmol, 0.90 equiv) dropwise at 0 degrees C. Then the mixture was stirred at 0 degrees C. for 2.0 h, then it was quenched by 2M HCl (100 mL). The mixture was extracted by EA (3×200 mL), the organic phases were combined and washed by sat. NaCl solution (300 mL), dried over anhydrous Na₂SO₄. The solid was filtered out and concentrated. The crude was dissolved in EtOH (300 mL), 6M HCl (100 mL) was added. The mixture was stirred at 85 degrees C. for 2.0 h. The solvent was removed, the residue was dissolved in water, adjusted the pH value to 9 with 2N NaOH. The mixture was extracted by EA (3×300 mL), the organic phases were combined and washed with sat. NaCl solution (1×300 mL), dried over anhydrous Na₂SO₄. The solid was filtered out and the filtration was concentrated. The residue was purified by silica gel column with PE/EA=3:1. This resulted 30.00 g of desired product as orange solid (58% yield). LC/MS: mass calcd. For C₁₄H₁₂ClNO₂: 261.06, found: 262.00 [M+H]⁺.

### Step 3: Synthesis of methyl (S)-3-((((9H-fluoren-9-yl)methoxy) carbonyl)amino)-4-((2-(4-chlorobenzoyl)-4-methoxyphenyl)amino)-4-oxobutanoate

To a solution of (2S)-2-[[(9H-fluoren-9-ylmethoxy)carbonyl]amino]-4-methoxy-4-oxobutanoic acid (6.21 g, 16.81 mmol, 1.10 eq) in DMF (50.00 mL) was added HATU (7.55 g, 19.87 mmol, 1.30 eq) and DIEA (5.93 g, 45.85 mmol, 3.00 eq). Then 2-(4-chlorobenzoyl)-4-methoxyaniline (4.00 g, 15.28 mmol, 1.00 eq) was added. The mixture was stirred at room temperature for 17 h. The mixture was poured into 300 mL ice/water, the solid was filtered out. Then the solid was dissolved in DCM and purified by silica gel column with PE:EA=3:1. Methyl (S)-3-((((9H-fluoren-9-yl)methoxy) carbonyl)amino)-4-((2-(4-chlorobenzoyl)-4-methoxyphenyl)amino)-4-oxobutanoate (5.00 g, 53.00%) was obtained as yellow solid. LC/MS: mass calcd. For C₃₄H₂₉ClN₂O₇: 612.17, found: 613.15 [M+H]⁺.

### Step 4: Synthesis of methyl (S)-2-(5-(4-chlorophenyl)-7-methoxy-2-oxo-2,3-dihydro-1H-benzo[e][1,4]diazepin-3-yl)acetate

To a solution of methyl (3S)-3-[[2-(4-chlorobenzoyl)-4-methoxyphenyl]carbamoyl]-3-[[(9H-fluoren-9-ylmethoxy)carbonyl]amino]propanoate (5.00 g, 8.16 mmol, 1.00 eq) in DCM (15.00 mL) was added Et₃N (15.00 mL). The mixture was stirred at 50 degrees C. for 5.0 h. The solvent was removed and the residue was dissolved in DCE (50.00 mL), AcOH (4.50 g, 73.404 mmol, 9.00 eq) was added. The mixture was stirred at 65 degrees C. for 2.0 h. The solvent was removed and the residue was purified with silica gel column with PE:EA=1:1. Methyl (S)-2-(5-(4-chlorophenyl)-7-methoxy-2-oxo-2,3-dihydro-1H-benzo[e][1,4]diazepin-3-yl)acetate (2.80 g, 78.00% yield) was obtained as yellow solid. LC/MS: mass calcd. For C₁₉H₁₇ClN₂O₄: 372.09, found: 373.15 [M+H]⁺.

### Step 5: Synthesis of methyl (S)-2-(5-(4-chlorophenyl)-7-methoxy-2-thioxo-2,3-dihydro-1H-benzo[e][1,4]diazepin-3-yl)acetate

Into a 250 mL flask was added P₂S₅(4.51 g, 20.28 mmol, 3.60 equiv), Na₂CO₃ (1.07 g, 0.01 mmol, 1.80 equiv), ClCH₂CH₂Cl (60.00 mL). The mixture was stirred at room temperature for 2 h, then methyl 2-[(3S)-5-(4-chlorophenyl)-7-methoxy-2-oxo-1,3-dihydro-1,4-benzodiazepin-3-yl]acetate (2.10 g, 5.63 mmol, 1.00 equiv) was added. The reaction was stirred at 65 degrees C. for 3 h, then the solid was filtered, the organic phase was washed with water (50 mL), dried by Na₂SO₄. The solid was filtered out and the filtration was concentrated. This resulted in methyl 2-[(3S)-5-(4-chlorophenyl)-7-methoxy-2-sulfanylidene-1,3-dihydro-1,4-benzodiazepin-3-yl]acetate (1.70 g, 63.53%) as a yellow solid. LC/MS: mass calcd. For C₁₉H₁₇ClN₂O₃S: 388.06, found: 389.10 [M+H]⁺.

### Step 6: Synthesis of methyl 2-[(2Z,3S)-5-(4-chlorophenyl)-2-(acetamidoimino)-7-methoxy-1,3-dihydro-1,4-benzodiazepin-3-yl]acetate

Into a 250 mL flask was added methyl 2-[(3S)-5-(4-chlorophenyl)-7-methoxy-2-sulfanylidene-1,3-dihydro-1,4-benzodiazepin-3-yl]acetate (1.50 g, 3.86 mmol, 1.00 equiv), THF (40.00 mL). The mixture was cooled to 0 degrees C., then NH₂NH₂.H₂O (0.58 g, 0.01 mmol, 3.00 equiv) was added dropwise, the mixture was stirred at 0˜5 degrees C. for 1 h. Then Et₃N (1.17 g, 0.01 mmol, 3.00 equiv) was added, AcCl (0.91 g, 0.01 mmol, 3.00 equiv) was added dropwise under 0-5 degrees C., the reaction was stirred at room temperature for 2 h. The reaction was quenched with H₂O at room temperature, extracted with EA (3×40 mL), the organic layers were combined and washed with brine (2×20 mL), dried over anhydrous Na₂SO₄. After filtration, the filtrate was concentrated under reduced pressure. This resulted in methyl 2-[(2Z,3S)-5-(4-chlorophenyl)-2-(acetamidoimino)-7-methoxy-1,3-dihydro-1,4-benzodiazepin-3-yl]acetate (1.70 g, crude) as yellow solid. LC/MS: mass calcd. For C₂₁H₂₁ClN₄O₄: 428.13, found: 429.15 [M+H]⁺.

### Step 7: Synthesis of methyl (S)-2-(6-(4-chlorophenyl)-8-methoxy-1-methyl-4H-benzo[f][1,2,4]triazolo[4,3-a][1,4]diazepin-4-yl)acetate

Into a 100 mL flask was added methyl 2-[(2Z,3S)-5-(4-chlorophenyl)-2-(acetamidoimino)-7-methoxy-1,3-dihydro-1,4-benzodiazepin-3-yl]acetate (1.77 g crude), THF (10.00 mL), AcOH (10.00 mL), the reaction was stirred at room temperature for 3 h. Then the reaction mixture was concentrated. The residue was added NaHCO₃/DCM mixture (20 mL/20 mL), the organic phase was separated and the aqueous phases were extracted with DCM (2×20 mL). The organic phases were combined and washed with water (20 ml), NaCl solution (20 ml) and dried over anhydrous Na₂SO₄. The solid was filtered out and the filtration was concentrated. The residue was purified by silica gel column chromatography, eluted with CH₂Cl₂:MeOH=20:1 to afford methyl (S)-2-(6-(4-chlorophenyl)-8-methoxy-1-methyl-4H-benzo[f][1,2,4]triazolo[4,3-a][1,4]diazepin-4-yl)acetate (1.58 g, 75.0% yield of two steps) as yellow solid. LC/MS: mass calcd. For C₂₁H₁₉ClN₄O₃: 410.11, found: 411.25 [M+H]⁺.

### Step 8: Synthesis of (S)-2-(6-(4-chlorophenyl)-8-methoxy-1-methyl-4H-benzo[f][1,2,4]triazolo[4,3-a][1,4]diazepin-4-yl)acetic acid (SM29)

Into a 100 mL flask was added methyl (S)-2-(6-(4-chlorophenyl)-8-methoxy-1-methyl-4H-benzo[f][1,2,4]triazolo[4,3-a][1,4]diazepin-4-yl)acetate (1.58 g, 3.85 mmol, 1.00 equiv), LiOH solution (2M, 6.00 mL, 12.00 mmol, 3.12 equiv), MeOH (20.00 mL), THF (10.00 mL). The reaction was stirred at room temperature for 2 h. Then the mixture was concentrated, the residue was dissolved with 30 mL water, cooled to 0 degrees C., adjust PH to 3-5 by 2M HCl. The purified solids were collected by filtration and washed with water (3×20 mL), dried under vacuum. This resulted in (S)-2-(6-(4-chlorophenyl)-8-methoxy-1-methyl-4H-benzo[f][1,2,4]triazolo[4,3-a][1,4]diazepin-4-yl)acetic acid (1.30 g, 80.62% yield) as yellow solid. If the crude was purified by chiral Prep-HPLC under the condition: Column: Reg-AD, 3*25 cm, 5μm; Mobile Phase A: CO₂, Mobile Phase B: MEOH (0.1% 2M NH₃-MEOH); Flow rate: 100 mL/min; Gradient: 30% B; UV: 220 nm; RT1: 3.05; RT2: 4.08. Pure SM29 could be obtained LC/MS: mass calcd. For C₂₀H₁₇ClN₄O₃: 396.10, found: 397.15 [M+H]⁺.

### Step 9: Synthesis of (S)—N-(4-((tert-butyldimethylsilyl)oxy)phenyl)-2-(6-(4-chlorophenyl)-8-methoxy-1-methyl-4H-benzo[f][1,2,4]triazolo[4,3-a][1,4]diazepin-4-yl)acetamide

Into a 250 mL flask was added aminophenol (2.00 g, 18.33 mmol, 1.00 equiv), THF (60.00 mL), imidazole (1.62 g, 0.02 mmol, 1.30 equiv). The mixture was cooled to 0 degrees C., then TBDMSCl (4.14 g, 0.03 mmol, 1.50 equiv) was added slowly, the reaction was stirred at room temperature for 1 h. The reaction was quenched with water (100 mL), extracted with EA (3×50 mL), the organic phase was washed with NaCl solution (50 mL), dried by Na₂SO₄, the solid was filtered out and the filtrate was concentrated. The residue was purified by silica gel column chromatography, eluted with PE:EA=1:1 to afford 4-[(tert-butyldimethylsilyl)oxy]aniline (3.80 g, 92.82%) as brown oil. LC/MS: mass calcd. For C₁₂H₂₁NOSi: 223.14, found: 224.15 [M+H]⁺.

Into a 100 mL flask was added (S)-2-(6-(4-chlorophenyl)-8-methoxy-1-methyl-4H-benzo[f][1,2,4]triazolo[4,3-a][1,4]diazepin-4-yl)acetic acid (500.00 mg, 1.26 mmol, 1.00 equiv), DMF (10.00 mL), HATU (718.63 mg, 1.89 mmol, 1.50 equiv), DIEA (651.38 mg, 5.04 mmol, 4.00 equiv). The mixture was stirred at room temperature for 10 mins, then 4-[(tert-butyldimethylsilyl)oxy]aniline (281.47 mg, 1.26 mmol, 1.00 equiv) was added. The reaction was stirred at room temperature for 1 h. Then the mixture was poured into ice water (20 mL), the solid was filtered out, washed with H₂O (5 mL) and dried under vacuum. This resulted in (S)—N-(4-((tert-butyldimethyl-silyl)oxy)phenyl)-2-(6-(4-chlorophenyl)-8-methoxy-1-methyl-4H-benzo[f][1,2,4]triazolo[4,3-a][1,4]diazepin-4-yl)acetamide (740.00 mg, 77.32%) as yellow solid. LC/MS: mass calcd. For C₃₂H₃₆ClN₅O₃Si: 601.23, found: 602.15 [M+H]⁺.

### Step 10: Synthesis of (S)-2-(6-(4-chlorophenyl)-8-methoxy-1-methyl-4H-benzo[f][1,2,4]triazolo[4,3-a][1,4]diazepin-4-yl)-N-(4-hydroxyphenyl)acetamide (SM10) and (R)-2-(6-(4-chlorophenyl)-8-methoxy-1-methyl-4H-benzo[f][1,2,4]triazolo[4,3-a][1,4]diazepin-4-yl)-N-(4-hydroxyphenyl)acetamide (SM20)

Into a 100 mL flask was added (S)—N-(4-((tert-butyldimethyl-silyl)oxy)phenyl)-1-(6-(4-chlorophenyl)-8-methoxy-1-methyl-4H-benzo[f][1,2,4] triazolo[4,3-a][1,4]diazepin-4-yl)acetamide (740.00 mg, 1.23 mmol, 1.00 equiv), DCM (10.00 mL), HF/Pyridine (0.55 mL, 6.11 mmol, 4.97 equiv). The reaction was stirred at room temperature for 3 h. The reaction was concentrated and the residue was purified by silica gel column chromatography, eluted with DCM:MeOH=20:1 to afford the crude product (380.00 mg). The crude product was purified by Perp-Chiral-HPLC under the condition: Column: CHIRALPAK IE, 2*25 cm, 5 um; Mobile Phase A: Hex (0.1% FA)-HPLC, Mobile Phase B: EtOH-HPLC; Flow rate: 17 mL/min; Gradient: 50 B to 50 B in 23 min; 254/220 nm; RT1: 10.619; RT2: 18.297. The fractions were combined and concentrated. (S)-2-(6-(4-chlorophenyl)-8-methoxy-1-methyl-4H-benzo[f][1,2,4]triazolo[4,3-a][1,4]diazepin-4-yl)-N-(4-hydroxyphenyl)acetamide (150 mg, 24.17%) and (R)-2-(6-(4-chlorophenyl)-8-methoxy-1-methyl-4H-benzo[f][1,2,4]triazolo[4,3-a][1,4]diazepin-4-yl)-N-(4-hydroxyphenyl)acetamide (70.00 mg, 11.10%) were obtained as yellow solid. LC/MS: mass calcd. For C₂₆H₂₂ClN₅O₃: 487.14, found: 488.10 [M+H]⁺.

### Synthesis of (S)-2-(6-(4-chlorophenyl)-1-methyl-4H-benzo[c]isoxazolo[4,5-e]azepin-4-yl)-N-(4-hydroxyphenyl)acetamide (SM11)

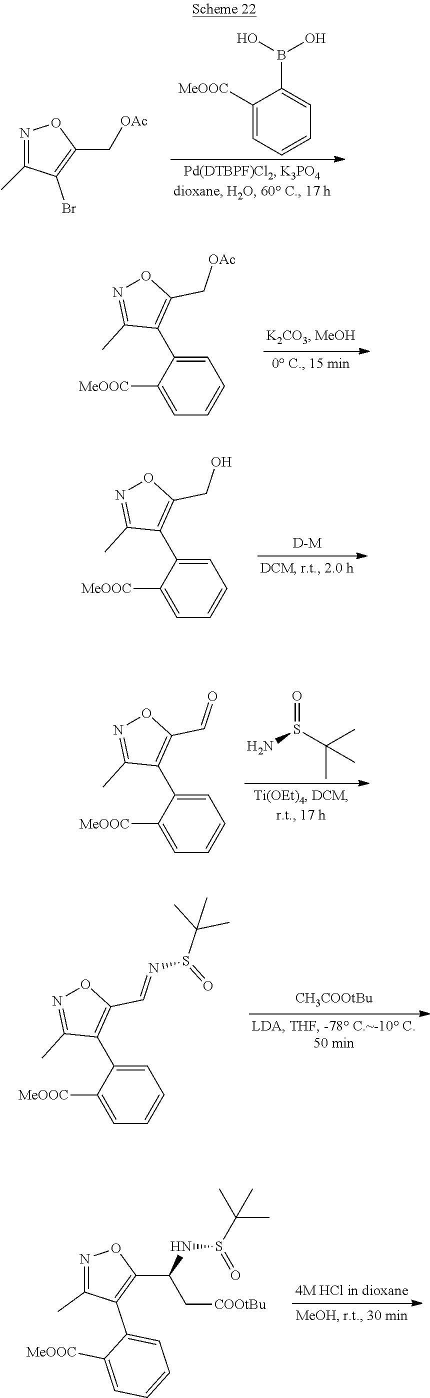

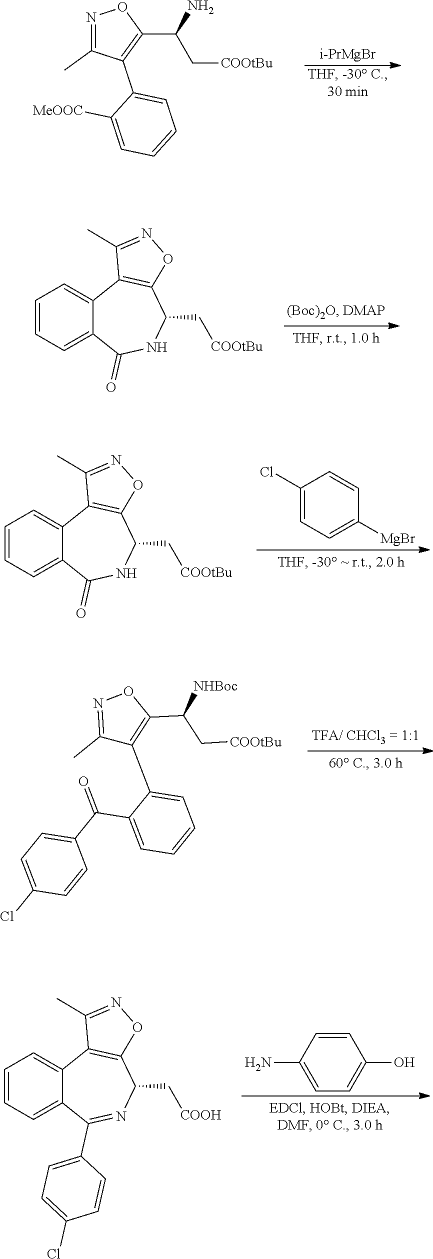

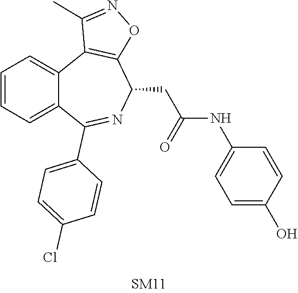 [SMILES: Cc1noc2c1-c1ccccc1C(c1ccc(Cl)cc1)=N[C@H]2CC(=O)Nc1ccc(O)cc1]

### Step 1: Synthesis of methyl 2-[5-[(acetyloxy)methyl]-3-methyl-1,2-oxazol-4-yl]benzoate

A solution of (4-bromo-3-methyl-1,2-oxazol-5-yl)methyl acetate (4.00 g, 17.09 mmol, 1.00 equiv), 2-(methoxycarbonyl)phenylboronic acid (3998.50 mg, 22.22 mmol, 1.30 equiv), K₃PO₄ (11136.81 mg, 34.18 mmol, 2.00 equiv) and Pd(DtBPF)Cl₂ (697.84 mg, 0.86 mmol, 0.05 equiv) in dioxane (20.00 mL) and H₂O (4.00 mL) was stirred at 60 degrees C. for 17 h under N₂ atmosphere. The solid was filtered out and the filter cake was washed with EA (10 mL). The filtrate and the EA solution were combined and concentrated.

The residue was purified by silica gel column chromatography, eluted with PE/EA=5:1 to afford methyl 2-[5-[(acetyloxy)methyl]-3-methyl-1,2-oxazol-4-yl]benzoate (1.90 g, 38.43%) as white solid. LCMS: mass calcd. For C₁₅H₁₅NO₅: 289.10, found: 290.20 [M+H]⁺.

### Step 2: Synthesis of methyl 2-[5-(hydroxymethyl)-3-methyl-1,2-oxazol-4-yl]benzoate

To a solution of methyl 2-[5-[(acetyloxy)methyl]-3-methyl-1,2-oxazol-4-yl]benzoate (1.90 g, 6.57 mmol, 1.00 equiv) in MeOH (40.00 mL) was added K₂CO₃ (1815.43 mg, 13.14 mmol, 2.00 equiv) at 0 degree C. Then the reaction was stirred at 0 degrees C. for 15 min. The mixture was diluted with H₂O (30 mL), extracted with DCM (3×50 mL). The organic phases were combined and washed with brine (50 mL), dried over Na₂SO₄. The solid was filtrated out and the filtration was concentrated to afford methyl 2-[5-(hydroxymethyl)-3-methyl-1,2-oxazol-4-yl]benzoate (1.40 g, 86.21%) as yellow oil. LCMS: mass calcd. For C₁₃H₁₃NO₄: 247.08, found: 248.20 [M+H]⁺.

### Step 3: Synthesis of methyl 2-(5-formyl-3-methyl-1,2-oxazol-4-yl)benzoate

To a solution of methyl 2-[5-(hydroxymethyl)-3-methyl-1,2-oxazol-4-yl]benzoate (1.40 g, 5.66 mmol, 1.00 equiv) in DCM (50.00 mL) was added Dess-martin (4803.23 mg, 11.33 mmol, 2.00 equiv). Then the reaction was stirred at room temperature for 2 h. The reaction was quenched with aq. Na₂S₂O₃ and aq. NaHCO₃ solution, extracted with DCM (3×50 mL). The organic phases were combined and washed with brine (50 mL), dried over Na₂SO₄. The solid was filtered out and the filtration was concentrated to afford methyl 2-(5-formyl-3-methyl-1,2-oxazol-4-yl) benzoate (1.50 g, crude) as yellow oil. LCMS: mass calcd. For C₁₃H₁₁NO₄: 245.07, found: 246.15 [M+H]⁺.

### Step 4: Synthesis of methyl 2-[3-methyl-5-([[(S)-2-methylpropane-2-sulfinyl]imino]methyl)-1,2-oxazol-4-yl]benzoate

To a solution of methyl 2-(5-formyl-3-methyl-1,2-oxazol-4-yl) benzoate (1.50 g, 6.12 mmol, 1.00 equiv) in DCM (15.00 mL) was added (S)-2-methylpropane-2-sulfinamide (889.60 mg, 7.34 mmol, 1.20 equiv) and tetraethoxytitanium (2790.53 mg, 12.23 mmol, 2.00 equiv). Then the reaction was stirred at room temperature for 17 h. The mixture was quenched with water (3 mL) and the solid was filtrated out. The filtrate was concentrated.

The residue was purified by silica gel column chromatography, eluted with PE/EA=1:1 to afford methyl 2-[3-methyl-5-([[(S)-2-methylpropane-2-sulfinyl]imino]methyl)-1,2-oxazol-4-yl]benzoate (1.70 g, 79.77%) as yellow solid. LCMS: mass calcd. For C₁₇H₂₀N₂O₄S: 348.11, found: 349.20 [M+H]⁺.

### Step 5: Synthesis of methyl 2-[5-[3-(tert-butoxy)-1-[[(S)-2-methylpropane-2-sulfinyl]amino]-3-oxopropyl]-3-methyl-1,2-oxazol-4-yl]benzoate

To a solution of tert-butyl acetate (364.00 mg, 3.13 mmol, 1.00 equiv) in THF (7.00 mL) was added LDA (3M in THF, 0.50 mL, 4.67 mmol, 1.49 equiv) dropwise at −78 degrees C. Then the reaction was stirred for 1 h allowing the temperature increased to −10 degrees C. Then the reaction mixture was cooled to −78 degree C., methyl 2-[3-methyl-5-([[(S)-2-methylpropane-2-sulfinyl]imino]methyl)-1,2-oxazol-4-yl]benzoate (1200.99 mg, 3.45 mmol, 1.10 equiv) in THF (10.00 mL) was added dropwise over 20 min. The resulting mixture was stirred for additional 30 mins with the temperature increased to −10 degrees C. The mixture was quenched with aq.NH₄Cl (5 mL), extracted with EA (3×15 mL). The organic phases were combined and washed with brine (25 mL), dried over Na₂SO₄. The solid was filtered out and the filtration was concentrated. The residue was purified by silica gel column with PE/EA=3:1 to afford methyl 2-[5-[3-(tert-butoxy)-1-[[(S)-2-methylpropane-2-sulfinyl]amino]-3-oxopropyl]-3-methyl-1,2-oxazol-4-yl]benzoate (1.00 g, 68.69%) as white solid. LCMS: mass calcd. For C₂₃H₃₂N₂O₆S: 464.20, found: 465.10 [M+H]⁺.

### Step 6: Synthesis of methyl (S)-2-(5-(1-amino-3-(tert-butoxy)-3-oxopropyl)-3-methylisoxazol-4-yl)benzoate

A solution of methyl 2-[5-[(1S)-3-(tert-butoxy)-1-[[(S)-2-methylpropane-2-sulfinyl]amino]-3-oxopropyl]-3-methyl-1,2-oxazol-4-yl]benzoate (1.20 g, 2.58 mmol, 1.00 equiv) in methanol (15.00 mL) was added 4M HCl in 1,4-dioxane (1.03 mL, 4.133 mmol, 1.60 equiv). The mixture was stirred at 0 degrees C. for 30 min. The mixture was concentrated to afford 1.00 g crude of methyl (S)-2-(5-(1-amino-3-(tert-butoxy)-3-oxopropyl)-3-methylisoxazol-4-yl)benzoate was obtained as yellow oil. LCMS: mass calcd. For C₁₉H₂₄N₂O₅: 360.17, found: 361.25 [M+H]⁺.

### Step 7: Synthesis of tert-butyl (S)-2-(1-methyl-6-oxo-5,6-dihydro-4H-benzo[c]isoxazolo[4,5-e]azepin-4-yl)acetate

A solution methyl 2-[5-[(1S)-1-amino-3-(tert-butoxy)-3-oxopropyl]-3-methyl-1,2-oxazol-4-yl]benzoate (840.00 mg, 2.33 mmol, 1.00 equiv) in THF (8.00 mL) was added bromo(isopropyl)magnesium (2.9 M in 2-Me-THF, 2.41 mL, 6.99 mmol, 3.00 equiv) dropwise under N₂ atmosphere. The reaction mixture was stirred for 30 mins at −30 degree C. Then the reaction was quenched with aq.NH₄Cl (15 mL), extracted with EA (3×15 mL). The organic phases were combined and washed with brine (15 mL), dried over Na₂SO₄. The solid was filtrated out and the filtration was concentrated. The residue was purified by silica gel column, eluted with PE/EA=2:1. The fractions were combined together and concentrated to afford tert-butyl (S)-2-(1-methyl-6-oxo-5,6-dihydro-4H-benzo[c]isoxazolo[4,5-e]azepin-4-yl)acetate (750.00 mg, 98.00%) as white solid. LCMS: mass calcd. For C₁₈H₂₀N₂O₄: 328.14, found: 329.10 [M+H]⁺.

### Step 8: Synthesis of tert-butyl (S)-4-(2-(tert-butoxy)-2-oxoethyl)-1-methyl-6-oxo-4,6-dihydro-5H-benzo [c]isoxazolo[4,5-e]azepine-5-carboxylate

To a solution of tert-butyl (S)-2-(1-methyl-6-oxo-5,6-dihydro-4H-benzo[c]isoxazolo[4,5-e]azepin-4-yl)acetate (720.00 mg, 2.19 mmol, 1.00 equiv) in THF (6.00 mL) was added Boc₂O (574.25 mg, 2.63 mmol, 1.20 equiv) and DMAP (13.39 mg, 0.11 mmol, 0.05 equiv). Then the reaction was stirred at room temperature for 1 h. The solvent was removed and the residue was purified by silica gel column, eluted with PE/EA=2:1 to afford tert-butyl (S)-4-(2-(tert-butoxy)-2-oxoethyl)-1-methyl-6-oxo-4,6-dihydro-5H-benzo[c]isoxazolo[4,5-e]azepine-5-carboxylate (800 mg, 85.15%) as yellow solid. LCMS: mass calcd. For C₂₃H₂₈N₂O₆:428.19, found: 451.10 [M+Na]⁺.

### Step 9: Synthesis of tert-butyl (3S)-3-[(tert-butoxycarbonyl)amino]-3-[4-[2-(4-chlorobenzoyl)phenyl]-3-methyl-1,2-oxazol-5-yl]propanoate

A solution of tert-butyl (S)-4-(2-(tert-butoxy)-2-oxoethyl)-1-methyl-6-oxo-4,6-dihydro-5H-benzo[c]isoxazolo[4,5-e]azepine-5-carboxylate (800.00 mg, 1.87 mmol, 1.00 equiv) in THF (10.00 mL) was added chloro(4-chlorophenyl)magnesium (1.0 M in THF, 2.24 mL, 2.24 mmol, 1.20 equiv) dropwise at −30 degree C. under N₂ atmosphere. The reaction mixture was warmed to room temperature naturally and stirred for 2.0 h. The mixture was quenched with aq. NH₄Cl (5 mL), extracted with EA (3×10 mL), washed brine (15 mL), dried over Na₂SO₄. The solid was filtered out and the filtration was concentrated. The residue was purified by silica gel column, eluted with PE/EA=4:1 to afford tert-butyl (3S)-3-[(tert-butoxycarbonyl)amino]-3-[4-[2-(4-chlorobenzoyl)phenyl]-3-methyl-1,2-oxazol-5-yl]propanoate (940.00 mg, 93.06%) as white solid. LCMS: mass calcd. For C₂₉H₃₃ClN₂O₆: 540.20, found: 541.20 [M+H]⁺.

### Step 10: Synthesis of (S)-2-(6-(4-chlorophenyl)-1-methyl-4H-benzo[c]isoxazolo [4,5-e]azepin-4-yl)acetic acid

A solution of tert-butyl (3S)-3-[(tert-butoxycarbonyl)amino]-3-[4-[2-(4-chlorobenzoyl)phenyl]-3-methyl-1,2-oxazol-5-yl]propanoate (930.00 mg, 1.72 mmol, 1.00 equiv) in TFA (4.00 mL) and CHCl₃ (4.00 mL) was stirred at 60 degree C. for 3 h. The mixture was concentrated to dryness to afford (S)-2-(6-(4-chlorophenyl)-1-methyl-4H-benzo[c]isoxazolo[4,5-e]azepin-4-yl)acetic acid (600.00 mg, 95.16%) as yellow oil. LC/MS: mass calcd. For C₂₀H₁₅ClN₂O₃: 366.08, found: 367.15 [M+H]⁺.

### Step 11: Synthesis of (S)-2-(6-(4-chlorophenyl)-1-methyl-4H-benzo[c] isoxazolo[4,5-e]azepin-4-yl)-N-(4-hydroxyphenyl)acetamide

To a solution of (S)-2-(6-(4-chlorophenyl)-1-methyl-4H-benzo[c]isoxazolo[4,5-e]azepin-4-yl)acetic acid (400.00 mg, 1.09 mmol, 1.00 equiv) in DMF (5.00 mL) was added HOBt (221.03 mg, 1.64 mmol, 1.50 equiv), EDCI (313.58 mg, 1.64 mmol, 1.50 equiv), 4-[(tert-butyldimethylsilyl)oxy]aniline (255.79 mg, 1.15 mmol, 1.05 equiv) and DIEA (281.88 mg, 2.18 mmol, 2.00 equiv). Then the reaction was stirred at 0 C for 3 h. the reaction mixture was purified by reverse flash chromatograph with the following conditions:

Then the reaction was stirred at 0 degrees C. for 3 h. The reaction mixture was purified by reverse flash chromatography column, C18 column; mobile phase, MeCN in water (0.05% TFA), 5% to 60% gradient in 30 min; detector, UV 254 nm. The fractions were combined and concentrated to afford desired product (600.00 mg). The crude product was purified by Prep-Chiral HPLC with the following conditions (Column: CHIRALPAK IE, 2*25 cm, 5 um; Mobile Phase A: Hex (0.1% FA)—HPLC, Mobile Phase B: EtOH—HPLC; Flow rate: 20 mL/min; Gradient: 20 B to 20 B in 23 min; 220/254 nm; RT1: 15.425; RT2: 20.514; Injection Volume: 0.8 ml; Number Of Runs: 14;). The fractions were combined and concentrated to afford (S)-2-(6-(4-chlorophenyl)-1-methyl-4H-benzo[c]isoxazolo[4,5-e]azepin-4-yl)-N-(4-hydroxyphenyl)acetamide (300.00 mg, 60.08%) as white solid. LC/MS: mass calcd. For C₂₆H₂₀ClN₃O₃: 457.17, found: 458.20 [M+H]⁺.

### Synthesis of 2,4-Dimethyl-6-[1-[(1S)-1-phenylethyl]-6-(piperidin-4-yl)imidazo[4,5-c]pyridin-2-yl]pyridazin-3-one (SM14)

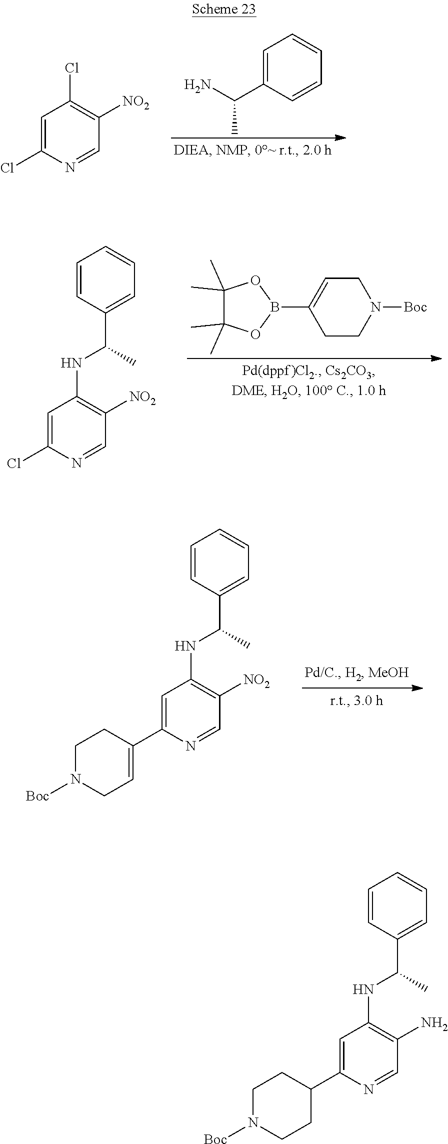

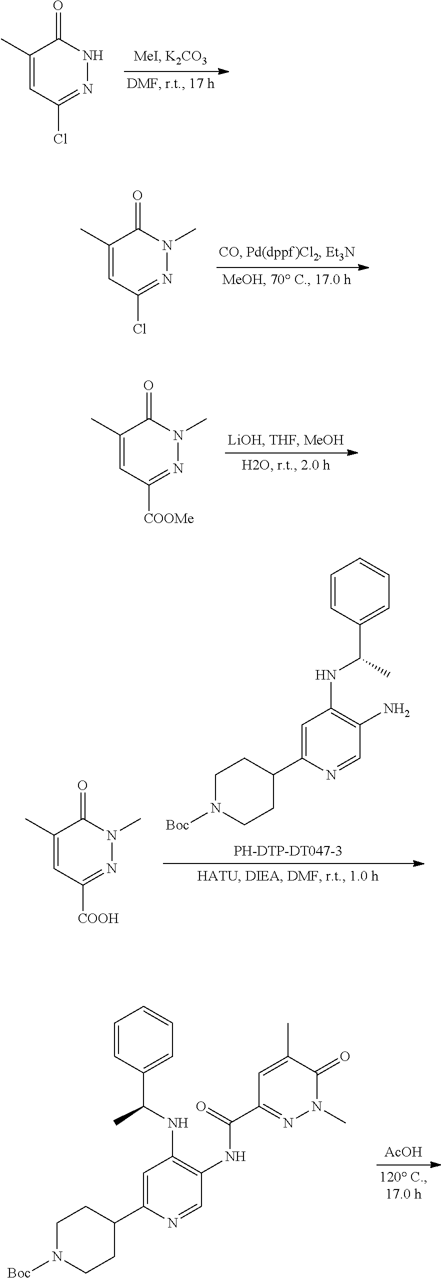

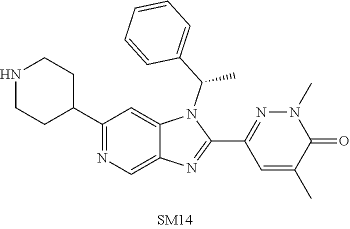 [SMILES: Cc1cc(-c2nc3cnc(C4CCNCC4)cc3n2[C@@H](C)c2ccccc2)nn(C)c1=O]

### Step 1: Synthesis of 2-Chloro-5-nitro-N-[(1S)-1-phenylethyl]pyridin-4-amine

To a stirred solution of 2,4-dichloro-5-nitropyridine (3.00 g, 15.55 mmol, 1.00 equiv) in NMP (80.00 mL) was added (S)-1-phenylethan-1-amine (1.87 g, 15.43 mmol, 0.90 equiv) and DIEA (6.02 g, 46.64 mmol, 3.00 equiv) dropwise at 0 degrees C. The resulting mixture was stirred for 2 h at room temperature. The reaction was quenched with ice/water (240 mL) at 0 degrees C. The resulting mixture was extracted with EA (3×300 mL). The combined organic layers were washed with aqueous NaCl (2×300 mL), dried over anhydrous Na₂SO₄. After filtration, the filtrate was concentrated under reduced pressure. 2-Chloro-5-nitro-N-[(1S)-1-phenylethyl] pyridin-4-amine (4.00 g, 83.39%) was obtained as yellow liquid. LC/MS: mass calcd. For C₁₃H₁₂ClN₃O₂: 277.06, found: 278.05 [M+H]⁺.

### Step 2: Synthesis of tert-butyl 5-nitro-4-[[(1S)-1-phenylethyl]amino]-3′,6′-dihydro-2′H-[2,4′-bipyridine]-1′-carboxylate

To a stirred mixture of 2-chloro-5-nitro-N-[(1S)-1-phenylethyl]pyridin-4-amine (4.00 g, 14.40 mmol, 1.00 equiv) and tert-butyl 4-(4,4,5,5-tetramethyl-1,3,2-dioxaborolan-2-yl)-3,6-dihydro-2H-pyridine-1-carboxylate (10.02 g, 32.41 mmol, 2.25 equiv) in DME (100.00 mL) and H₂O (25.00 mL) was added Cs₂CO₃ (9.39 g, 28.81 mmol, 2.00 equiv) and Pd(dppf)Cl₂ (1.05 g, 1.44 mmol, 0.10 equiv) at room temperature. Then N₂ was exchanged three times. The resulting mixture was stirred for 1 h at 100 degrees C. under N₂ atmosphere. The resulting mixture was diluted with water (50 mL). The resulting mixture was extracted with EA (3×200 mL). The combined organic layers were washed with aqueous NaCl (2×200 mL), dried over anhydrous Na₂SO₄. After filtration, the filtrate was concentrated under reduced pressure. The residue was purified by silica gel column chromatography, eluted with PE:EA=3:1 to afford tert-butyl 5-nitro-4-[[(1S)-1-phenylethyl]amino]-3′,6′-dihydro-2′H-[2,4′-bipyridine]-1′-carboxylate (5.50 g, 89.95%) as yellow oil. LC/MS: mass calcd. For C₂₃H₂₈N₄O₄: 424.21, found: 425.25 [M+H]⁺.

### Step 3: Synthesis of tert-butyl 4-(5-amino-4-[[(1S)-1-phenylethyl]amino] pyridine-2-yl)piperidine-1-carboxylate

To a stirred solution of tert-butyl 5-nitro-4-[[(1S)-1-phenylethyl]amino]-3′,6′-dihydro-2′H-[2,4′-bipyridine]-1′-carboxylate (3.00 g, 7.07 mmol, 1.00 equiv) in MeOH (40.00 mL) was added Pd/C (300.00 mg, 10% w/w) at room temperature. Then H₂ was exchanged by three times. The resulting mixture was stirred for 3 h at room temperature under H₂ atmosphere. The resulting mixture was filtered, the filter cake was washed with MeOH (3×20 mL). The filtrate was concentrated under reduced pressure. Tert-butyl 4-(5-amino-4-[[(1S)-1-phenylethyl]amino]pyridine-2-yl)piperidine-1-carboxylate (2.50 g, 89.21%) was obtained as red oil. LC/MS: mass calcd. For C₂₃H₃₂N₄O₂: 396.25, found: 397.25 [M+H]⁺.

### Step 4: Synthesis of 6-Chloro-2,4-dimethylpyridazin-3-one

To a stirred mixture of 6-chloro-4-methyl-2H-pyridazin-3-one (2.20 g, 15.22 mmol, 1.00 equiv) and K₂CO₃ (4.21 g, 30.44 mmol, 2.00 equiv) in DMF (40.00 mL) was added Mel (2.38 g, 16.77 mmol, 1.10 equiv) dropwise at room temperature. The resulting mixture was stirred for 17 h at room temperature. The reaction was quenched with ice/water (120 mL) at 0 degrees C. The resulting mixture was extracted with EA (3×200 mL). The combined organic layers were washed with NaCl (3×200 mL), dried over anhydrous Na₂SO₄. After filtration, the filtrate was concentrated under reduced pressure. 6-Chloro-2,4-dimethylpyridazin-3-one (2.00 g, 82.87%) was obtained as pink solid. LC/MS: mass calcd. For C₆H₇ClN₂O: 158.02, found: 159.05 [M+H]⁺.

### Step 5: Synthesis of methyl 1,5-dimethyl-6-oxopyridazine-3-carboxylate

To a stirred solution of 6-chloro-2,4-dimethylpyridazin-3-one (1.70 g, 10.72 mmol, 1.00 equiv) in MeOH (50.00 mL) was added Et₃N (2.71 g, 26.80 mmol, 2.50 equiv) and Pd(dppf)Cl₂.CH₂Cl₂ (262.62 mg, 0.32 mmol, 0.03 equiv) at room temperature. Then CO was exchanged by three times. The resulting mixture was stirred for 17 h at 70 degrees C. under CO atmosphere. The resulting mixture was concentrated under reduced pressure. The residue was purified by silica gel column chromatography, eluted with PE:EA=3:1 to afford methyl 1,5-dimethyl-6-oxopyridazine-3-carboxylate (1.90 g, 95.82%) as light yellow solid. LC/MS: mass calcd. For C₈H₁₀N₂O₃: 182.07, found: 183.10 [M+H]⁺.

### Step 6: Synthesis of 1,5-Dimethyl-6-oxopyridazine-3-carboxylic acid

To a stirred solution of methyl 1,5-dimethyl-6-oxopyridazine-3-carboxylate (1.70 g, 9.33 mmol, 1.00 equiv) in THF (4.00 mL) and MeOH (4.00 mL) was added LiOH (670.41 mg, 28.00 mmol, 3.00 equiv) in H₂O (4.00 mL) dropwise. The resulting mixture was stirred for 2 h at room temperature. The resulting mixture was concentrated under reduced pressure. Then the residue was dissolved in H₂O (5 mL). The mixture was acidified to pH 4˜5 with 2N HCl. The precipitated solids were collected by filtration and washed with H₂O (3×5 mL), dried under vacuum. 1,5-Dimethyl-6-oxopyridazine-3-carboxylic acid (1.40 g, 89.22%) was obtained as white solid. LC/MS: mass calcd. For C₇H₈N₂O₃: 168.05, found: 169.05 [M+H]⁺.

### Step 7: Synthesis of tert-butyl 4-[5-(1,5-dimethyl-6-oxopyridazine-3-amido)-4-[[(1S)-1-phenylethyl]amino]pyridin-2-yl]piperidine-1-carboxylate

To a stirred solution of 1,5-dimethyl-6-oxopyridazine-3-carboxylic acid (650.00 mg, 3.87 mmol, 1.00 equiv) in DMF (30.00 mL) was added HATU (1.76 g, 4.64 mmol, 1.20 equiv), DIEA (1.50 g, 11.60 mmol, 3.00 equiv) and tert-butyl 4-(5-amino-4-[[(1S)-1-phenylethyl]amino]pyridin-2-yl)piperidine-1-carboxylate (1.84 g, 4.64 mmol, 1.20 equiv) at room temperature. The resulting mixture was stirred for 1 h at room temperature. The reaction was quenched with water/ice (90 mL) at 0 degrees C. The precipitated solid were collected by filtration and washed with H₂O (3×20 mL), dried under vacuum. Tert-butyl 4-[5-(1,5-dimethyl-6-oxopyridazine-3-amido)-4-[[(1S)-1-phenylethyl]amino]pyridin-2-yl]piperidine-1-carboxylate (1.30 g, 61.52%) was obtained as pink solid. LC/MS: mass calcd. For C₃₀H₃₈N₆O₄: 546.30, found: 547.40 [M+H]⁺.

### Step 8: Synthesis of 2,4-Dimethyl-6-[1-[(1S)-1-phenylethyl]-6-(piperidin-4-yl) imidazo[4,5-c]pyridin-2-yl]pyridazin-3-one

A solution of tert-butyl 4-[5-(1,5-dimethyl-6-oxopyridazine-3-amido)-4-[[(1S)-1-phenylethyl]amino]pyridine-2-yl]piperidine-1-carboxylate (1.00 g, 1.83 mmol, 1.00 equiv) in acetic acid (10.00 mL) was stirred for 17 h at 120 degrees C. The resulting mixture was concentrated under vacuum. The residue was purified by reverse flash chromatography with the following conditions: column, C18 column; mobile phase, ACN in water (0.05% TFA), 20% to 60% gradient in 40 min; detector, UV 254 nm. The fractions were combined and concentrated. 300 mg crude SM14 was obtained. Then it was purified by Prep-HPLC under the condition: Column: XBridge C18 OBD Prep Column, 100A, 10 μm, 19 mm×250 mm; Mobile Phase A: Water (10 MMOL/L NH₄HCO₃+0.1% NH₃.H₂O), Mobile Phase B: MeOH—HPLC; Flow rate: 25 mL/min; Gradient: 27 B to 55 B in 16.5 min; 220 nm; RT1: 14.55. The fractions were combined and concentrated. 2,4-Dimethyl-6-[1-[(1S)-1-phenylethyl]-6-(piperidin-4-yl)imidazo[4,5-c]pyridin-2-yl]pyridazin-3-one (80.00 mg, 9.16%) was obtained as yellow oil. LC/MS: mass calcd. For C₂₅H₂₈N₆O: 428.23, found: 429.15 [M+H]⁺.

### Synthesis of 4-(2-(3-((4-aminobenzyl)amino)phenoxy)-5-(methylsulfonyl)phenyl)-6-methyl-1,6-dihydro-7H-pyrrolo[2,3-c]pyridin-7-one (SM16)

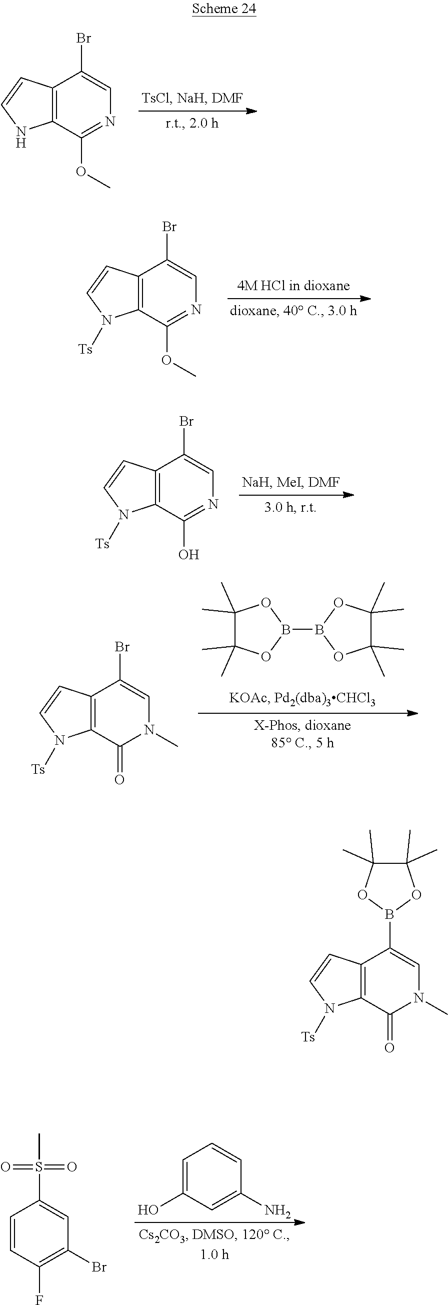

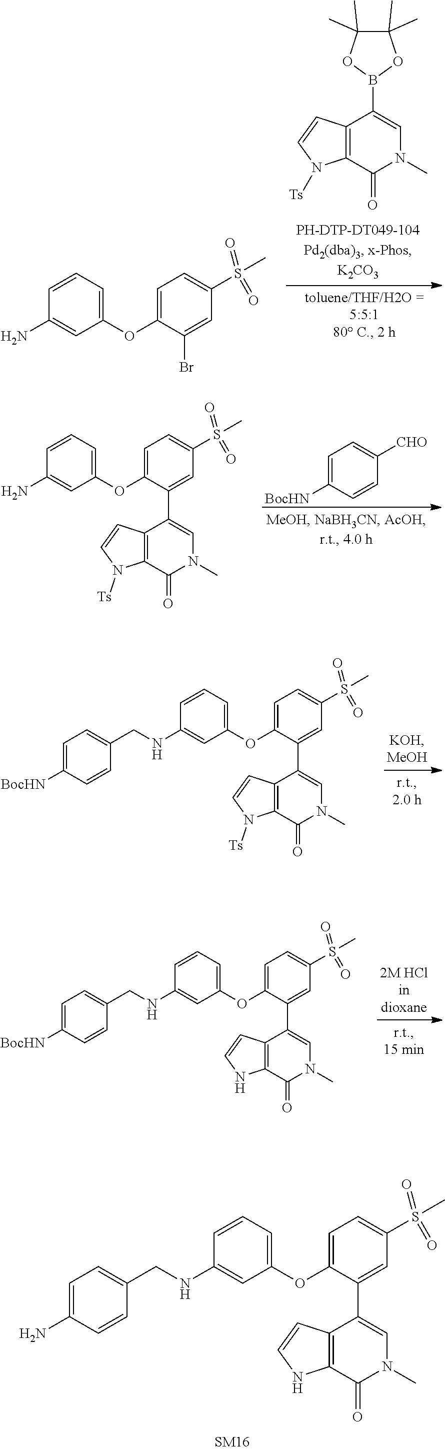

### Step 1: Synthesis of 4-bromo-7-methoxy-1-(4-methylbenzenesulfonyl)pyrrolo[2,3-c]pyridine

To a solution of 4-bromo-7-methoxy-1H-pyrrolo[2,3-c]pyridine (2.00 g, 8.81 mmol, 1.00 equiv) in DMF (10.00 mL) was added NaH (60%) (317.07 mg, 13.21 mmol, 1.50 equiv) in portions at 0 degrees C. Then the reaction was stirred for 15 min followed by addition of TsCl (2518.89 mg, 13.21 mmol, 1.50 equiv) at 0 degrees C. The resulting mixture was stirred for additional 2 h at room temperature. The mixture was poured into ice water (50 mL), the solid was filtrated, washed by H₂O (10 mL) and dried to afford 4-bromo-7-methoxy-1-(4-methylbenzenesulfonyl)pyrrolo[2,3-c]pyridine (3.50 g, 100%) as white solid. LCMS: mass calcd. For C₁₅H₁₃BrN₂O₃S: 379.98, 381.98, found: 381.05, 383.05 [M+H, M+2+H]⁺.

### Step 2: Synthesis of 4-bromo-1-(4-methylbenzenesulfonyl)pyrrolo[2,3-c] pyridin-7-ol

A solution of 4-bromo-7-methoxy-1-(4-methylbenzenesulfonyl)pyrrolo[2,3-c] pyridine (4.00 g, 10.47 mmol, 1.00 equiv) in HCl/1,4-dioxane (4M, 40.00 mL) was stirred at 40 degrees C. for 3.0 h. The mixture was concentrated to dryness to afford 4-bromo-1-(4-methylbenzenesulfonyl)pyrrolo[2,3-c]pyridin-7-ol (3.50 g crude) as yellow solid. LCMS: mass calcd. For C₁₄H₁₁BrN₂O₃S: 365.97, 367.97, found: 365.05, 367.05 [M+H, M+2+H]⁺.

### Step 3: Synthesis of 4-bromo-6-methyl-1-(4-methylbenzenesulfonyl)pyrrolo[2,3-c]pyridin-7-one

To a stirred solution of 4-bromo-1-(4-methylbenzenesulfonyl)pyrrolo[2,3-c]pyridine-7-ol (3.50 g, 9.53 mmol, 1.00 equiv) in DMF (30.00 mL) was added NaH (60%) (0.27 g, 11.44 mmol, 1.20 equiv) in portions at 0 degree C. under under N₂ atmosphere. The mixture was stirred for 10 mins at 0 degree C., then Mel (1.62 g, 11.44 mmol, 1.20 equiv) was added dropwise at 0 degree C. The mixture was stirred for 3.0 h at room temperature. Then it was poured into 100 mL ice/water. The solid was filtered out, washed with water (10 mL), and dried over vacuum. 4-bromo-6-methyl-1-(4-methylbenzenesulfonyl)pyrrolo[2,3-c]pyridin-7-one (3.30 g, 90.82% yield) was obtained as white solid. LCMS: mass calcd. For C₁₅H₁₃BrN₂O₃S: 379.98, 381.98, found: 380.05, 381.05 [M+H, M+2+H]⁺.

### Step 4: Synthesis of 6-methyl-1-(4-methylbenzenesulfonyl)-4-(4,4,5,5-tetramethyl-1,3,2-dioxaborolan-2-yl)pyrrolo[2,3-c]pyridin-7-one

To a solution of 4-bromo-6-methyl-1-(4-methylbenzenesulfonyl)pyrrolo [2,3-c]pyridin-7-one (2.00 g, 5.25 mmol, 1.00 equiv) in dioxane (60.00 mL) was added bis(pinacolato)diboron (2664.36 mg, 10.50 mmol, 2.00 equiv), KOAc (1132.69 mg, 11.54 mmol, 2.20 equiv), Pd₂(dba)₃.CHCl₃ (271.51 mg, 0.26 mmol, 0.05 equiv) and X-Phos (250.09 mg, 0.53 mmol, 0.10 equiv) at room temperature under N₂ atmosphere. The resulting mixture was stirred for 5 h at 85 degrees C. under N₂ atmosphere. The mixture was concentrated, dissolved in EA (40 mL) and H₂O (40 mL), extracted with EA (3×40 mL). The organic phases were combined and dried over Na₂SO₄. The solid was filtrated out and the filtrate was concentrated. The residue was purified by silica gel column chromatography, eluted with PE/EA=1:1 to afford 6-methyl-1-(4-methylbenzenesulfonyl)-4-(4,4,5,5-tetramethyl-1,3,2-dioxaborolan-2-yl)pyrrolo[2,3-c]pyridin-7-one (1.40 g, 62.31%) as yellow solid. LCMS: mass calcd. For C₂₁H₂₅BN₂O₅S: 428.16, found: 429.20 [M+H]⁺.

### Step 5: Synthesis of 3-(2-bromo-4-methanesulfonylphenoxy)aniline

To a solution of 2-bromo-1-fluoro-4-methanesulfonylbenzene (700.00 mg, 2.77 mmol, 1.00 equiv) in DMSO (15.00 mL) was added m-aminophenol (362.19 mg, 3.32 mmol, 1.20 equiv) and Cs₂CO₃ (1.11 g, 3.41 mmol, 1.50 equiv). Then the reaction was stirred at 120 degrees C. for 1 h. The reaction mixture was poured into 30 mL ice-water, extracted with EA (3×25 mL). The organic phases were combined and washed with brine (2×50 ml), dried over Na₂SO₄. The solid was filtrated out and the filtrate was concentrated. The residue was purified by silica gel column chromatography, eluted with PE:EA=1:2 to afford 3-(2-bromo-4-methanesulfonylphenoxy)aniline (0.90 g, 95.43% yield) as a yellow solid. LCMS: mass calcd. For C₁₃H₁₂BrNO₃S: 340.97, 342.97, found: 341.95, 343.95 [M+H, M+2+H]⁺.

### Step 6: Synthesis of 4-[2-(3-aminophenoxy)-5-methanesulfonylphenyl]-6-methyl-1H-pyrrolo[2,3-c]pyridin-7-one

To a solution of 3-(2-bromo-4-methanesulfonylphenoxy)aniline (400.00 mg, 1.17 mmol, 1.00 equiv) in dioxane (10.00 mL), toluene (10.00 mL) and H₂O (2.00 mL) was added Pd₃(dba)₂ (76.18 mg, 0.12 mmol, 0.10 equiv), K₂CO₃ (496.22 mg, 2.34 mmol, 2.00 equiv) and 6-methyl-4-(4,4,5,5-tetramethyl-1,3,2-dioxaborolan-2-yl)-1H-pyrrolo[2,3-c]pyridin-7-one (352.47 mg, 1.29 mmol, 1.10 equiv). Then the reaction was stirred for 2 h at 80 degrees C. Then the solid was filtered out and the filtrate was concentrated. The solid was purified by silica gel column chromatography, eluted with DCM:MeOH=15:1 to afford 4-[2-(3-aminophenoxy)-5-methanesulfonylphenyl]-6-methyl-1H-pyrrolo[2,3-c]pyridin-7-one (600.00 mg, 91.14%) as yellow solid. LCMS: mass calcd. For C₂₈H₂₅N₃O₆S₂: 563.12, found: 564.25 [M+H]⁺.

### Step 7: Synthesis of tert-butyl N-(4-[[(3-[4-methanesulfonyl-2-[6-methyl-1-(4-methylbenzenesulfonyl)-7-oxopyrrolo[2,3-c]pyridin-4-yl]phenoxy]phenyl) amino]methyl]phenyl)carbamate

To a solution of 4-[2-(3-aminophenoxy)-5-methanesulfonylphenyl]-6-methyl-1-(4-methylbenzenesulfonyl)pyrrolo[2,3-c]pyridin-7-one (900.00 mg, 1.60 mmol, 1.00 equiv) in MeOH (20.00 mL) was added tert-butyl N-(4-formylphenyl)carbamate (353.29 mg, 1.60 mmol, 1.00 equiv), NaBH₃CN (120.41 mg, 1.92 mmol, 1.20 equiv) and AcOH (191.78 mg, 3.19 mmol, 2.00 equiv). Then the reaction was stirred at room temperature for 4 h. The mixture was quenched with aq. NH₄Cl (25 mL), extracted with EA (3×20 mL), washed brine (50 mL), dried Na₂SO₄. The solid was filtrated out and the filtrate was concentrated. The residue was purified by silica gel column chromatography, eluted with DCM:MeOH=20:1 to afford tert-butyl N-(4-[[(3-[4-methanesulfonyl-2-[6-methyl-1-(4-methylbenzenesulfonyl)-7-oxopyrrolo[2,3-c]pyridin-4-yl]phenoxy]phenyl)amino]methyl]phenyl)carbamate (900.00 mg, 73.30%) as yellow solid. LCMS: mass calcd. For C₄₀H₄₀N₄O₈S₂: 768.23, found: 769.40 [M+H]⁺.

### Step 8: Synthesis of tert-butyl N-[4-([[3-(4-methanesulfonyl-2-[6-methyl-7-oxo-1H-pyrrolo[2,3-c]pyridin-4-yl]phenoxy)phenyl]amino]methyl)phenyl]carbamate

To a solution of tert-butyl N-(4-[[(3-[4-methanesulfonyl-2-[6-methyl-1-(4-methylbenzenesulfonyl)-7-oxopyrrolo[2,3-c]pyridin-4-yl]phenoxy]phenyl)amino]methyl]phenyl)carbamate (900.00 mg, 1.17 mmol, 1.00 equiv) in methanol (10.00 mL) was added KOH (262.68 mg, 4.68 mmol, 4.00 equiv). Then the reaction was stirred at room temperature for 2 h. The mixture was filtrated and the filtrate was concentrated. The residue was purified by reverse flash chromatography with the following conditions: column, C18 column; mobile phase, MeCN in water (0.05%), 5% to 50% gradient in 30 min; detector, UV 254 nm. The fractions were combined and concentrated to afford tert-butyl N-[4-([[3-(4-methanesulfonyl-2-[6-methyl-7-oxo-1H-pyrrolo[2,3-c]pyridin-4-yl]phenoxy)phenyl]amino]methyl)phenyl]carbamate (400.00 mg, 55.59%) as white solid. LCMS: mass calcd. For C₃₃H₃₄N₄O₆S: 614.22, found: 615.40 [M+H]⁺.

### Step 9: Synthesis of 4-(2-(3-((4-aminobenzyl)amino)phenoxy)-5-(methylsulfonyl)phenyl)-6-methyl-1,6-dihydro-7H-pyrrolo[2,3-c]pyridin-7-one

A solution of tert-butyl N-[4-([[3-(4-methanesulfonyl-2-[6-methyl-7-oxo-1H-pyrrolo[2,3-c]pyridin-4-yl]phenoxy)phenyl]amino]methyl)phenyl]carbamate (200.00 mg) in HCl/1,4-dioxane (2.00 mL, 2M) was stirred at room temperature for 15 mins. The mixture was concentrated under reduced pressure to afford 4-(2-(3-((4-aminobenzyl)amino)phenoxy)-5-(methylsulfonyl)phenyl)-6-methyl-1,6-dihydro-7H-pyrrolo[2,3-c]pyridin-7-one (200 mg, crude) as yellow oil. It was used in next step without further purification. LCMS: mass calcd. For C₂₈H₂₆N₄O₄S: 514.17, found: 537.25 [M+Na]⁺.

### Synthesis of (R)-4-(8-methoxy-1-(1-methoxypropan-2-yl)-2-(piperidin-4-yl)-1H-imidazo [4,5-c]quinolin-7-yl)-3,5-dimethylisoxazole (SM17-Piperidine Linkage)

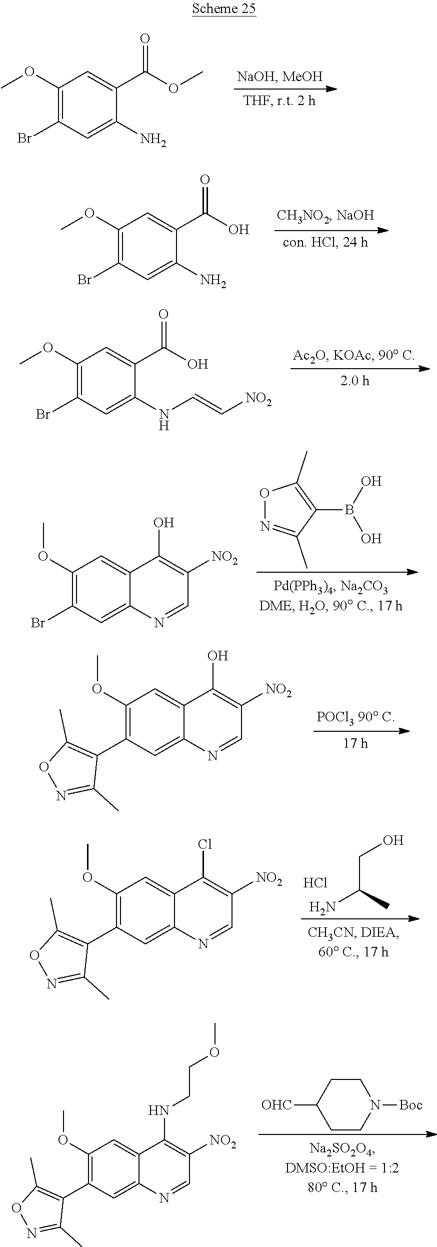

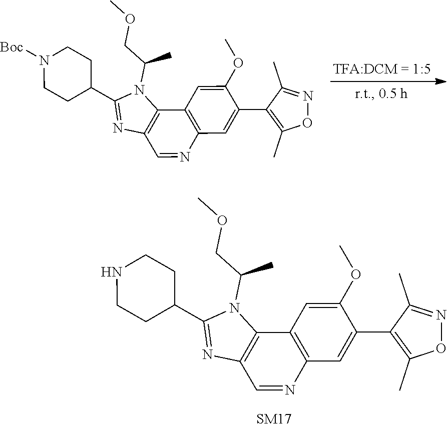 [SMILES: COC[C@@H](C)n1c(C2CCN(C(=O)OC(C)(C)C)CC2)nc2cnc3cc(-c4c(C)noc4C)c(OC)cc3c21 ; COC[C@@H](C)n1c(C2CCNCC2)nc2cnc3cc(-c4c(C)noc4C)c(OC)cc3c21]

### Step 1: Synthesis of 2-amino-4-bromo-5-methoxybenzoic acid

Into a 250 mL flask was added methyl 2-amino-4-bromo-5-methoxybenzoate (5.00 g, 18.24 mmol, 1.00 equiv), LiOH.H₂O (2.0 M, 36.00 mL, 72.00 mmol, 3.95 equiv), MeOH (70.00 mL), THF (36.00 mL). The reaction was stirred at room temperature for 2 h. The mixture was concentrated, the residue was dissolved with 50 ml water, cooled to 0 degrees C., adjust pH to 3-5 by 2M HCl, the precipitated solids were collected by filtration and washed with water (3×20 mL), dried under vacuum. This resulted in 2-amino-4-bromo-5-methoxybenzoic acid (4.00 g, 86.88%) as white solid. LC/MS: mass calcd. For C₈H₈BrNO₃: 244.97, 246.97, found: 246.10, 248.10 [M+H, M+2+H]⁺.

### Step 2: Synthesis of 4-bromo-5-methoxy-2-[[(E)-2-nitroethenyl] amino] benzoic acid

Into a 250 mL flask was added NaOH (6.18 g, 154.43 mmol, 10.00 equiv), H₂O (20.00 mL), the solution was cooled to 0 degrees C., then CH₃NO₂(9.43 g, 154.43 mmol, 10.00 equiv) was added dropwise, then the solution was stirred at 70 degrees C. for 20 mins, it turned to brown, then cooled to 0 degrees C., H₂O (20 mL) was added, the solution was adjust to pH=2-3 by con. HCl. Into another 250 mL flask was added 2-amino-4-bromo-5-methoxybenzoic acid (3.80 g, 15.44 mmol, 1.00 equiv), HCl/H₂O (10.00 mL: 60.00 mL). Then the above solution was added. The mixture was stirred at room temperature for 24 h. The precipitated solids were collected by filtration, washed with water (3×10 mL) and dried under vacuum. This resulted in 4-bromo-5-methoxy-2-[[(E)-2-nitroethenyl]amino]benzoic acid (4.46 g, 89.35%) as a yellow solid. LC/MS: mass calcd. For C₁₀H₉BrN₂O₅: 315.97, 317.94, found: 316.95, 318.95 [M+H, M+2+H]⁺.

### Step 3: Synthesis of 7-bromo-6-methoxy-3-nitroquinolin-4-ol

Into a 250 mL flask was added 4-bromo-5-methoxy-2-[[(E)-2-nitroethenyl]amino]benzoic acid (4.91 g, 15.48 mmol, 1.00 equiv), Ac₂O (60.00 mL), KOAc (1.82 g, 0.02 mmol, 1.20 equiv), the reaction was stirred at 90 degrees C. for 2 h. The reaction was quenched with water 60 mL. The solid was filtered out and washed with AcOH (2×20 mL), dried under vacuum. This resulted 7-bromo-6-methoxy-3-nitroquinolin-4-ol (4.30 g, 84.67%) as brown solid. LC/MS: mass calcd. For C₁₀H₇BrN₂O₄: 297.96, 299.96, found: 298.95, 300.95 [M+H, M+2+H]⁺.

### Step 4: Synthesis of 7-(3,5-dimethyl-1,2-oxazol-4-yl)-6-methoxy-3-nitroquinolin-4-ol

Into a 500 mL flask was added 7-bromo-6-methoxy-3-nitroquinolin-4-ol (4.30 g, 14.38 mmol, 1.00 equiv), 3,5-dimethyl-1,2-oxazol-4-ylboronic acid (3.05 g, 21.53 mmol, 1.50 equiv), Na₂CO₃ (4.57 g, 0.04 mmol, 3.00 equiv), DME (60.00 mL), H₂O (30.00 mL), then Pd(PPh₃)₄ (1.66 g, 2.15 mmol, 0.10 equiv) was added, the reaction was stirred at 90 degrees C. under N₂ atmosphere for 17 h. The reaction was concentrated and dissolved in EA (50 mL), the solid was filtered. The filtration was extracted by EA (3×30 mL), the organic phases were combined and dried over anhydrous Na₂SO₄. The solid was filtered out and the filtration was concentrated and the residue was purified by silica gel column chromatography, eluted with CH₂Cl₂: MeOH=20:1 to afford 7-(3,5-dimethyl-1,2-oxazol-4-yl)-6-methoxy-3-nitroquinolin-4-ol (3.60 g, 62.58%) as yellow solid. LC/MS: mass calcd. For C₁₅H₁₃N₃O₅: 315.09, found: 316.10 [M+H]⁺.

### Step 5: Synthesis of 4-chloro-7-(3,5-dimethyl-1,2-oxazol-4-yl)-6-methoxy-3-nitroquinoline

Into a 100 ml flask was added 7-(3,5-dimethyl-1,2-oxazol-4-yl)-6-methoxy-3-nitroquinolin-4-ol (1.00 g, 3.17 mmol, 1.00 equiv), POCl₃ (10.00 mL, 107.28 mmol, 33.82 equiv), the reaction was stirred at 90 degrees C. for 17 h. The reaction was concentrated, the residue was purified by silica gel column chromatography, eluted with PE:EA=1:1 to afford 4-chloro-7-(3,5-dimethyl-1,2-oxazol-4-yl)-6-methoxy-3-nitroquinoline (0.80 g, 61.43%) as yellow oil. LC/MS: mass calcd. For C₁₅H₁₂ClN₃O₄: 333.05, found: 334.05 [M+H]⁺.

### Step 6: Synthesis of (R)-7-(3,5-dimethylisoxazol-4-yl)-6-methoxy-N-(1-methoxypropan-2-yl)-3-nitroquinolin-4-amine

Into a 100 mL flask was added 4-chloro-7-(3,5-dimethyl-1,2-oxazol-4-yl)-6-methoxy-3-nitroquinoline (1.30 g, 3.90 mmol, 1.00 equiv), CH₃CN (30.00 mL), (2R)-1-methoxypropan-2-amine hydrochloride (0.73 g, 5.84 mmol, 1.50 equiv), DIEA (1.51 g, 11.69 mmol, 3.00 equiv). The reaction was stirred at 60 degrees C. for 17 h. The reaction was concentrated, the residue was purified by silica gel column chromatography, eluted with PE:EA=3:1 to afford (R)-7-(3,5-dimethylisoxazol-4-yl)-6-methoxy-N-(1-methoxypropan-2-yl)-3-nitroquinolin-4-amine (1.48 g, 94.69%) as yellow solid. LC/MS: mass calcd. For C₁₉H₂₂N₄O₅: 386.16, found: 387.25 [M+H]⁺.

### Step 7: Synthesis of tert-butyl 4-[7-(3,5-dimethyl-1,2-oxazol-4-yl)-8-methoxy-1-[(2R)-1-methoxypropan-2-yl]imidazo[4,5-c]quinolin-2-yl]piperidine-1-carboxylate

Into a 20 mL tube was added 7-(3,5-dimethyl-1,2-oxazol-4-yl)-6-methoxy-N-[(2R)-1-methoxypropan-2-yl]-3-nitroquinolin-4-amine (400.00 mg, 1.04 mmol, 1.00 equiv), tert-butyl 4-formylpiperidine-1-carboxylate (264.93 mg, 1.24 mmol, 1.20 equiv), Na₂S₂O₄ (67.59 mg, 0.39 mmol, 3.00 equiv), DMSO (4.00 mL), EtOH (8.00 mL). The reaction was stirred at 80 degrees C. for 17 h. The reaction was quenched with water (15 mL), extracted with EA (3×15 mL), washed with water (25 mL), NaCl solution (25 mL), dried with Na₂SO₄. The solid was filtered out and the filtration was concentrated, the residue was purified by TLC-Plat with DCM:MeOH=20:1. This resulted in tert-butyl 4-[7-(3,5-dimethyl-1,2-oxazol-4-yl)-8-methoxy-1-[(2R)-1-methoxypropan-2-yl]imidazo[4,5-c]quinolin-2-yl]piperidine-1-carboxylate (390.00 mg, 61.69%) as an off-white solid. LC/MS: mass calcd. For C₃₀H₃₉N₅O₅: 549.30, found: 550.40 [M+H]⁺.

### Step 8: Synthesis of 4-[7-(3,5-dimethyl-1,2-oxazol-4-yl)-8-methoxy-1-[(2R)-1-methoxypropan-2-yl] imidazo[4,5-c]quinolin-2-yl]piperidine

Into a 25 mL flask was added tert-butyl 4-[7-(3,5-dimethyl-1,2-oxazol-4-yl)-8-methoxy-1-[(2R)-1-methoxypropan-2-yl]imidazo[4,5-c]quinolin-2-yl]piperidine-1-carboxylate (390.00 mg, 0.71 mmol, 1.00 equiv), DCM (5.00 mL), TFA (1.00 mL). The reaction was stirred at room temperature for 0.5 h. The reaction was concentrated. This resulted in 4-[7-(3,5-dimethyl-1,2-oxazol-4-yl)-8-methoxy-1-[(2R)-1-methoxypropan-2-yl]imidazo[4,5-c]quinolin-2-yl]piperidine (320.00 mg, 83.69%) as yellow oil. LC/MS: mass calcd. For C₂₅H₃₁N₅O₃: 449.24, found: 450.25 [M+H]⁺.

### Synthesis of 7-(3,5-dimethyl-1,2-oxazol-4-yl)-1-[(2R)-1-methoxypropan-2-yl]-2-(oxan-4-yl)imidazo[4,5-c]quinolin-8-ol (SM17-Phenol-Linkage)

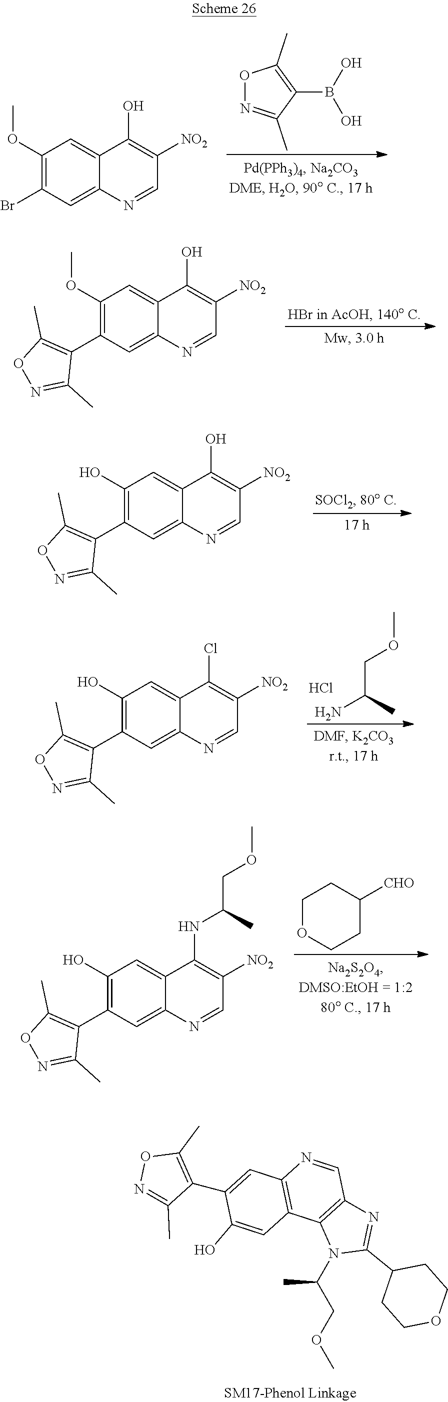

### Step 1: Synthesis of 7-(3,5-dimethyl-1,2-oxazol-4-yl)-6-methoxy-3-nitroquinolin-4-ol

Into a 500 mL flask was added 7-bromo-6-methoxy-3-nitroquinolin-4-ol (2.00 g, 6.69 mmol, 1.00 equiv), 3,5-dimethyl-1,2-oxazol-4-ylboronic acid (1.41 g, 10.04 mmol, 1.50 equiv), Na₂CO₃ (4.25 g, 40.12 mmol, 6.00 equiv), DME (60.00 mL), H₂O (30.00 mL), then Pd(PPh₃)₄ (0.77 g, 0.67 mmol, 0.10 equiv) was added, the reaction was stirred at 90 degrees C. under N₂ atmosphere for 17 h. The reaction was concentrated and dissolved in EA (50 mL), the solid was filtered. The filtrate was concentrated and the residue was purified by silica gel column chromatography, eluted with DCM:MeOH=20:1 to afford 7-(3,5-dimethyl-1,2-oxazol-4-yl)-6-methoxy-3-nitroquinolin-4-ol (1.50 g, 71.2%) as yellow solid. LC/MS: mass calcd. For C₁₅H₁₃N₃O₅: 315.09, found: 316.10 [M+H]⁺.

### Step 2: Synthesis of 7-(3,5-dimethyl-1,2-oxazol-4-yl)-3-nitroquinoline-4,6-diol

Into a 50 mL tubes was added 7-(3,5-dimethyl-1,2-oxazol-4-yl)-6-methoxy-3-nitroquinolin-4-ol (600.00 mg, 1.90 mmol, 1.00 equiv), 33% HBr in AcOH (20.00 mL), the reaction was stirred at 140 degrees C. under a microwave reactor for 3 h. The reaction was concentrated and the residue was purified by reverse flash chromatography with the following conditions: column, C18 column; mobile phase, MeCN in water (0.05% TFA), 10% to 50% gradient in 40 min; detector, 254 nm. The fractions were combined and concentrated. This resulted in 7-(3,5-dimethyl-1,2-oxazol-4-yl)-3-nitroquinoline-4,6-diol (600.00 mg, 83.72%) as yellow solid. LC/MS: mass calcd. For C₁₄H₁₁N₃O₅: 301.07, found: 302.15 [M+H]⁺.

### Step 3: Synthesis of 4-chloro-7-(3,5-dimethyl-1,2-oxazol-4-yl)-3-nitroquinolin-6-ol

Into a 50 mL flask was added 7-(3,5-dimethyl-1,2-oxazol-4-yl)-3-nitroquinoline-4,6-diol (300.00 mg, 1.00 mmol, 1.00 equiv), SOCl₂ (10.00 mL), the reaction was stirred at 80 degrees C. for 17 h. The reaction was quenched with ice/water (10 mL), extracted with EA (3×10 mL), the organic phase was washed with brine (20 mL), dried by Na₂SO₄. After filtration, the filtrate was concentrated. This resulted in 4-chloro-7-(3,5-dimethyl-1,2-oxazol-4-yl)-3-nitroquinolin-6-ol (300.00 mg crude) as yellow solid. LC/MS: mass calcd. For C₁₄H₁₀ClN₃O₄: 319.04, found: 320.10 [M+H]⁺.

### Step 4: Synthesis of 7-(3,5-dimethyl-1,2-oxazol-4-yl)-4-[[(2R)-1-methoxypropan-2-yl]amino]-3-nitroquinolin-6-ol

Into a 50 ml flask was added 4-chloro-7-(3,5-dimethyl-1,2-oxazol-4-yl)-3-nitroquinolin-6-ol (160.00 mg, 0.50 mmol, 1.00 equiv), DMF (5.00 mL), (2R)-1-methoxypropan-2-amine hydrochloride (94.29 mg, 0.75 mmol, 1.50 equiv), K₂CO₃ (276.67 mg, 2.00 mmol, 4.00 equiv), the reaction was stirred at room temperature for 17 h. Then K₂CO₃ was filtered out, the filtrate (6.00 mL DMF) was purified by reverse flash chromatography with the following conditions: column, C18 column; mobile phase, MeOH in water (0.05% TFA), 10% to 50% gradient in 10 min; UV 254 nm. The fractions were combined and concentrated. This resulted in 7-(3,5-dimethyl-1,2-oxazol-4-yl)-4-[[(2R)-1-methoxypropan-2-yl]amino]-3-nitroquinolin-6-ol (170.00 mg, 72.97%) as yellow oil. LC/MS: mass calcd. For C₁₈H₂₀N₄O₅: 372.14, found: 373.15 [M+H]⁺.

### Step 5: Synthesis of 7-(3,5-dimethyl-1,2-oxazol-4-yl)-1-[(2R)-1-methoxypropan-2-yl]-2-(oxan-4-yl)imidazo[4,5-c]quinolin-8-ol

Into a 20 ml tube was added 7-(3,5-dimethyl-1,2-oxazol-4-yl)-4-[[(2R)-1-methoxypropan-2-yl]amino]-3-nitroquinolin-6-ol (170.00 mg, 0.46 mmol, 1.00 equiv), DMSO (2.00 mL), EtOH (4.00 mL), oxane-4-carbaldehyde (62.53 mg, 0.55 mmol, 1.20 equiv), Na₂S₂O₄ (238.45 mg, 1.37 mmol, 3.00 equiv). The reaction was stirred at 80 degrees C. for 17 h. The reaction was quenched with water (20 mL). The resulting mixture was extracted with EtOAc (3×20 mL). The combined organic layers were combined and washed with brine (1×20 mL), dried over anhydrous Na₂SO₄. After filtration, the filtrate was concentrated under reduced pressure. The residue was purified by silica gel column chromatography, eluted with PE:EA=1:1 to afford 7-(3,5-dimethyl-1,2-oxazol-4-yl)-1-[(2R)-1-methoxypropan-2-yl]-2-(oxan-4-yl)imidazo[4,5-c]quinolin-8-ol (190.00 mg, 95.34%) as a yellow oil. LC/MS: mass calcd. For C₂₄H₂₈N₄O₄: 436.21, found: 437.30 [M+H]⁺.

### Synthesis of 2-(3-hydroxy-5-methylphenyl)-5,7-dimethoxy-3H-quinazolin-4-one (SM21)

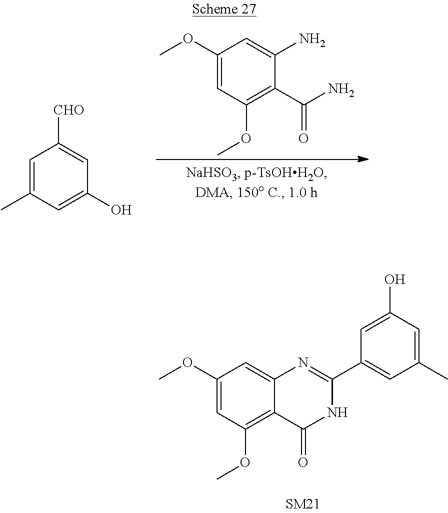

### Step 1: Synthesis of 2-(3-hydroxy-5-methylphenyl)-5,7-dimethoxy-3H-quinazolin-4-one

Into a 100 ml flask was added 3-hydroxy-5-methylbenzaldehyde (500.00 mg, 3.67 mmol, 1.00 equiv), 2-amino-4,6-dimethoxybenzamide (720.55 mg, 3.67 mmol, 1.00 equiv), NaHSO₃ (382.16 mg, 3.67 mmol, 1.00 equiv), TsOH.H₂O (69.86 mg, 0.37 mmol, 0.10 equiv), DMA (15.00 mL), the reaction was stirred at 150 degrees C. for 1 h. The reaction was quenched with MeCN (20 mL), white solid was separated out and obtained, the solvent was concentrated and purified by reverse flash chromatography with the following conditions: column, C18 column; mobile phase, ACN in water (0.05% TFA), 10% to 50% gradient in 30 min; detector, UV 254 nm. The fractions were combined and concentrated. This resulted in 2-(3-hydroxy-5-methylphenyl)-5,7-dimethoxy-3H-quinazolin-4-one (1.00 g, 83.62%) as white solid. LC/MS: mass calcd. For C₁₇H₁₆N₂O₄: 312.32, found: 313.05 [M+H]⁺. ¹H NMR (400 MHz, DMSO-d₆) δ: 11.88 (s, 1H), 9.61 (s, 1H), 7.44 (s, 1H), 7.36 (t, J=2.1 Hz, 1H), 6.78 (s, 1H), 6.72 (d, J=2.3 Hz, 1H), 6.54 (d, J=2.4 Hz, 1H), 3.90 (s, 3H), 3.85 (s, 3H), 2.30 (s, 3H).

### Synthesis of (R)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide (SM24)

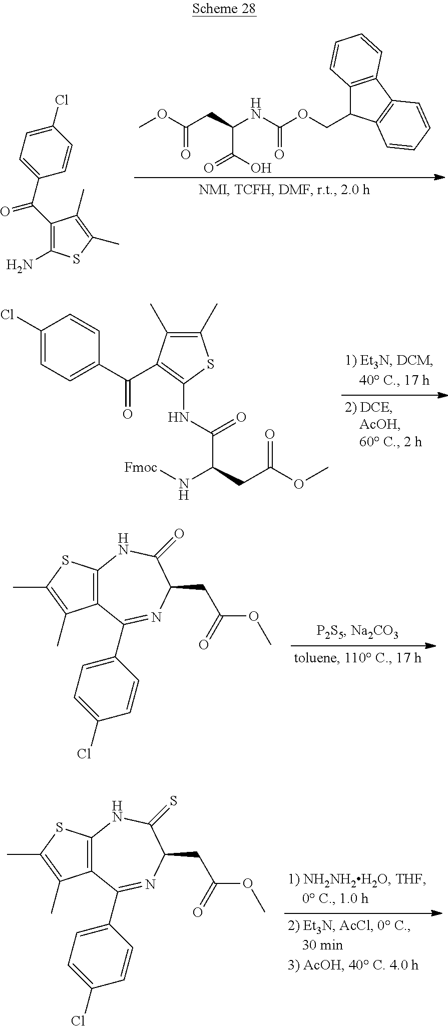

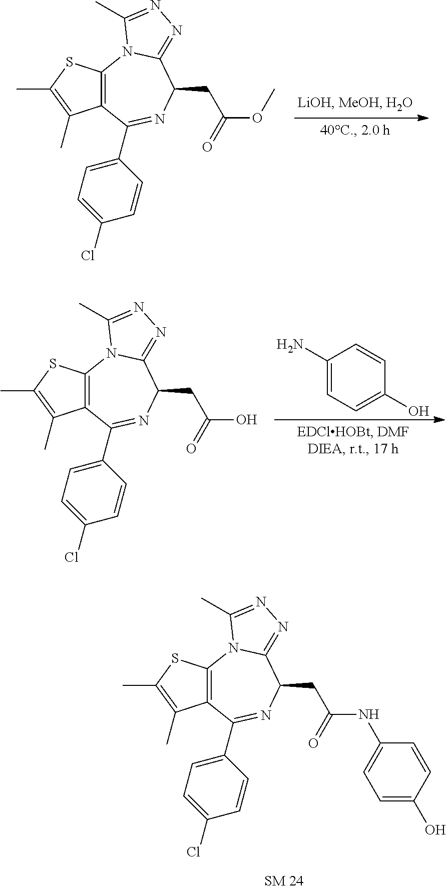

### Step 1: Synthesis of methyl (3R)-3-[[3-(4-chlorobenzoyl)-4,5-dimethylthiophen-2-yl]carbamoyl]-3-[[(9H-fluoren-9-ylmethoxy)carbonyl]amino]propanoate

To a stirred solution of (2R)-2-[[(9H-fluoren-9-ylmethoxy)carbonyl]amino]-4-methoxy-4-oxobutanoic acid (5.00 g, 13.54 mmol, 1.00 equiv) in DMF (60.00 mL) was added NMI (3.33 g, 40.61 mmol, 3.00 equiv), TCFH (5.70 g, 20.31 mmol, 1.50 equiv) and 3-(4-chlorobenzoyl)-4,5-dimethylthiophen-2-amine (3.96 g, 14.90 mmol, 1.10 equiv) in portions at 0 degrees C. The resulting mixture was stirred for 2 h at room temperature. The reaction was poured into water/ice (200 mL). The precipitated solids were collected by filtration and washed with water (3×30 mL), dried under vacuum. The solid was purified by silica gel column chromatography, eluted with PE/EA (3:1) to afford methyl (3R)-3-[[3-(4-chlorobenzoyl)-4,5-dimethylthiophen-2-yl]carbamoyl]-3-[[(9H-fluoren-9-ylmethoxy)carbonyl]amino]propanoate (4.95 g, 56.00%) as yellow solid. LCMS: mass calcd. For C₃₃H₂₉ClN₂O₆S: 616.14, found: 617.05 [M+H]⁺.

### Step 2: Synthesis of methyl 2-[(3R)-5-(4-chlorophenyl)-6,7-dimethyl-2-oxo-1H,3H-thieno[2,3-e][1,4]diazepin-3-yl]acetate

The procedure was the same as methyl (S)-2-(5-(4-chlorophenyl)-7-methoxy-2-oxo-2,3-dihydro-1H-benzo[e][1,4]diazepin-3-yl)acetate. 4.95 g of methyl (3R)-3-amino-3-[[3-(4-chlorobenzoyl)-4,5-dimethylthiophen-2-yl]carbamoyl]propanoate was used, 2.10 g of methyl 2-[(3R)-5-(4-chlorophenyl)-6,7-dimethyl-2-oxo-1H,3H-thieno[2,3-e][1,4]diazepin-3-yl]acetate was obtained as yellow solid (70.00% yield of two steps). LCMS: mass calcd. For C₁₈H₁₇ClN₂O₃S: 376.06, found: 377.15 [M+H]⁺.

### Step 3: Synthesis of methyl 2-[(3R)-5-(4-chlorophenyl)-6,7-dimethyl-2-sulfanylidene-1H,3H-thieno[2,3-e][1,4]diazepin-3-yl]acetate

To a solution of methyl 2-[(3R)-5-(4-chlorophenyl)-6,7-dimethyl-2-oxo-1H,3H-thieno[2,3-e][1,4]diazepin-3-yl]acetate (1.10 g, 2.92 mmol, 1.00 equiv) in toluene (25.00 ml) was added P₂S₅(1.30 g, 0.006 mmol, 2.00 equiv) and Na₂CO₃ (0.62 g, 5.85 mmol, 2.00 equiv). Then the reaction was stirred at 110 degrees C. for 17 h. The solid was filtered out and the filtration was concentrated. The residue was purified by silica gel column chromatography, eluted with PE:EA=1:1 to afford methyl 2-[(3R)-5-(4-chlorophenyl)-6,7-dimethyl-2-sulfanylidene-1H,3H-thieno[2,3-e][1,4]diazepin-3-yl]acetate (2 g, crude) as yellow solid. LCMS: mass calcd. For C₁₈H₁₇ClN₂O₂S₂: 392.04, found: 393.10 [M+H]⁺.

### Step 4: Synthesis of methyl (R)-2-(4-(4-chlorophenyl)-2,3,9-trimethyl-6H-thieno [3,2-f][1,2,4]triazolo[4,3-a][1,4]diazepin-6-yl)acetate

To a solution of methyl 2-[(3R)-5-(4-chlorophenyl)-6,7-dimethyl-2-sulfanylidene-1H,3H-thieno[2,3-e][1,4]diazepin-3-yl]acetate (900.00 mg, 2.29 mmol, 1.00 equiv) in THF (6.00 mL) was added NH₂NH₂.H₂O (344.00 mg, 6.87 mmol, 3.00 equiv) dropwise at 0 degrees C. Then the reaction was stirred. AcCl (539.41 mg, 6.87 mmol, 3.00 equiv) and Et₃N (695.34 mg, 6.87 mmol, 3.00 equiv) was added to the mixture and stirred at 0 degrees C. for 30 mins. The mixture was quenched with H₂O (15 mL), extracted with EA (3×15 mL), washed with brine (20 mL), dried over Na₂SO₄. The solid was filtrated out and the filtration was concentrated to dryness. The crude was dissolved in AcOH (10.0 mL) and stirred at 40 degrees C. for 4 h. The solvent was removed and the residue was purified by reverse flash chromatography with the following conditions: column, C18 column; mobile phase, MeCN in water (0.05% TFA), 5% to 50% gradient in 40 min; detector, UV 254 nm. The fractions were combined and concentrated to afford methyl (R)-2-(4-(4-chlorophenyl)-2,3,9-trimethyl-6H-thieno[3,2-f][1,2,4]triazolo[4,3-a][1,4]diazepin-6-yl)acetate(400.00 mg, 42.09%) as yellow solid. LCMS: mass calcd. For C₂₀H₁₉ClN₄O₂S: 414.09, found: 415.10 [M+H]⁺.

### Step 5: Synthesis of (R)-2-(4-(4-chlorophenyl)-2,3,9-trimethyl-6H-thieno[3,2-f][1,2,4]triazolo[4,3-a][1,4]diazepin-6-yl)acetic acid

A solution of methyl (R)-2-(4-(4-chlorophenyl)-2,3,9-trimethyl-6H-thieno[3,2-f][1,2,4]triazolo[4,3-a][1,4]diazepin-6-yl)acetate (400.00 mg, 0.96 mmol, 1.00 equiv) in LiOH (2M, 2.00 mL, 4.00 equiv) and MeOH (2.00 mL) was stirred at 40 degrees C. for 2 h. The mixture was acidified with HCl (2M) and concentrated. The residue was dissolved in DMF (2 mL) and purified by reverse phase column with the following conditions: column, C18 column; mobile phase, MeCN in water (0.05% TFA), 5% to 50% gradient in 40 min; detector, UV 254 nm. The fractions were combined and concentrated to afford (R)-2-(4-(4-chlorophenyl)-2,3,9-trimethyl-6H-thieno[3,2-f][1,2,4]triazolo[4,3-a][1,4]diazepin-6-yl)acetic acid (340.00 mg, 87.97% yield) as white solid. LCMS: mass calcd. For C₁₉H₁₇ClN₄O₂S: 400.09, found: 401.10 [M+H]⁺.

### Step 6: Synthesis of (R)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide

To a solution of (R)-2-(4-(4-chlorophenyl)-2,3,9-trimethyl-6H-thieno[3,2-f][1,2,4]triazolo[4,3-a][1,4]diazepin-6-yl)acetic acid (340.00 mg, 0.85 mmol, 1.00 equiv) in DMF (4.00 mL) was added EDCI (243.88 mg, 1.27 mmol, 1.50 equiv), HOBt (171.90 mg, 1.27 mmol, 1.50 equiv), aminophenol (101.81 mg, 0.93 mmol, 1.10 equiv) and DIEA (328.85 mg, 2.54 mmol, 3.00 equiv). Then the reaction was stirred at room temperature for 17 h. The reaction mixture was purified by reverse flash chromatography with the following conditions: column, C18 column; mobile phase, MeCN in water (0.05% TFA), 5% to 50% gradient in 40 min; detector, UV 254 nm. The fractions were combined and concentrated to afford crude product (380 mg). Then it was purified by Prep-chiral-HPLC with the following conditions (Column: CHIRALPAK IA, 2*25 cm, 5 um; Mobile Phase A: Hex (8 mmol/L NH₃. MeOH)—HPLC, Mobile Phase B: EtOH—HPLC; Flow rate: 18 mL/min; Gradient: 50 B to 50 B in 23 min; 254/220 nm; RT1: 6.708; RT2: 14.304;). The fractions were combined and concentrated to afford (R)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide (190.00 mg, 45.53%) as white solid. LCMS: mass calcd. For C₂₅H₂₂ClN₅O₂S: 491.12, found: 492.15 [M+H]⁺.

### Synthesis of (R)-7-(3,5-dimethylisoxazol-4-yl)-8-methoxy-1-(1-(pyridin-2-yl)ethyl)-1,3-dihydro-2H-imidazo[4,5-c]quinolin-2-one (SM25)

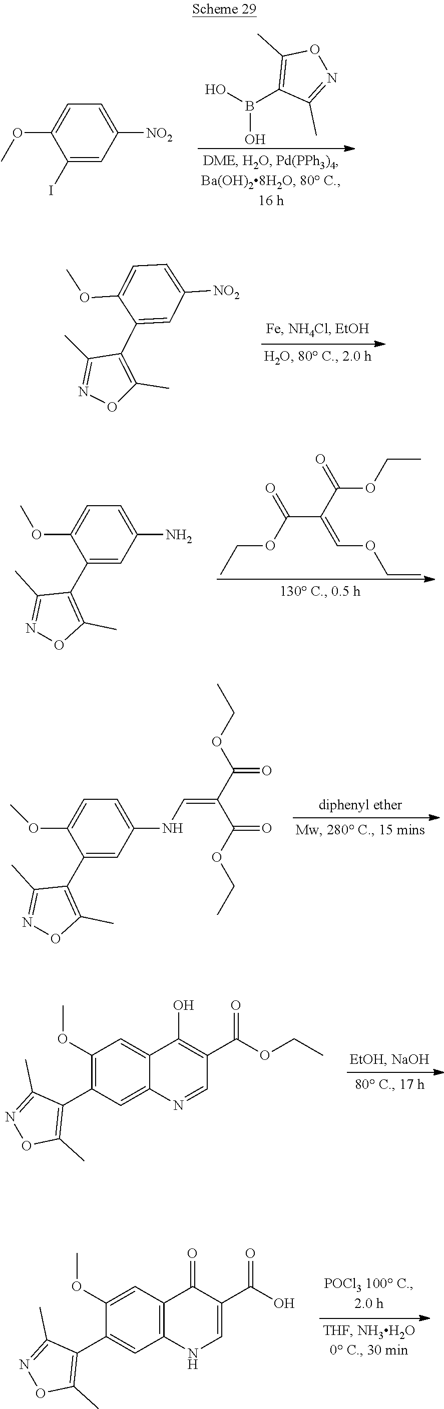

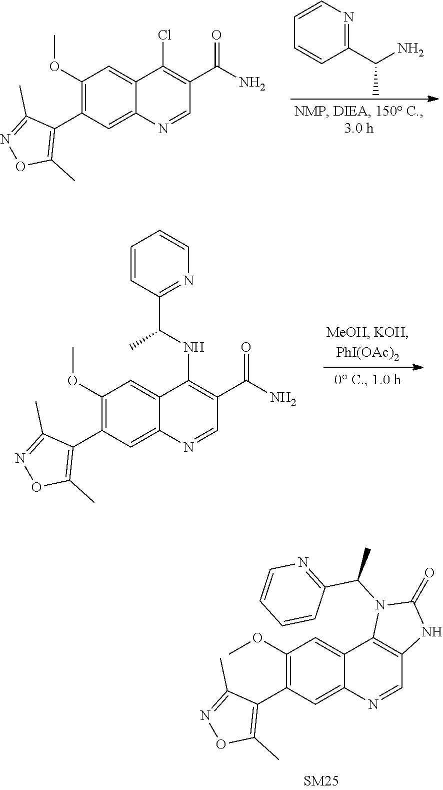

### Step 1: Synthesis of 4-(2-methoxy-5-nitrophenyl)-3,5-dimethyl-1,2-oxazole

Into a 500 mL flask was added 2-iodo-1-methoxy-4-nitrobenzene (7.50 g, 26.88 mmol, 1.00 equiv), DME (160.00 mL), H₂O (30.00 mL), (3,5-dimethylisoxazol-4-yl)boronic acid (11.36 g, 80.636 mmol, 3.00 equiv), Ba(OH)₂.8H₂O (16.93 g, 53.746 mmol, 2.00 equiv), Pd(PPh₃)₄(3.11 g, 2.69 mmol, 0.10 equiv). The reaction was stirred at 80 degrees C. for 16 h under N₂ atmosphere. The solid was filtered out, the resulting mixture was extracted with DCM (3×50 mL). The combined organic layers were combined and washed with NaHCO₃ solution (50 mL), water (50 mL), dried over anhydrous Na₂SO₄. After filtration, the filtrate was concentrated under reduced pressure. This result in 4-(2-methoxy-5-nitrophenyl)-3,5-dimethyl-1,2-oxazole (6.50 g, 75.43%) as red solid. LC/MS: mass calcd. For C₁₂H₁₂N₂O₄: 248.08, found: 249.05 [M+H]⁺.

### Step 2: Synthesis of 3-(3,5-dimethyl-1,2-oxazol-4-yl)-4-methoxyaniline

Into a 250 ml flask was added 4-(2-methoxy-5-nitrophenyl)-3,5-dimethyl-1,2-oxazole (6.50 g, 26.19 mmol, 1.00 equiv), EtOH (100.00 mL), H₂O (20.00 mL), NH₄Cl (2.80 g, 52.37 mmol, 2.00 equiv), Fe powder (8.77 g, 157.11 mmol, 6.00 equiv), the reaction was stirred at 80 degrees C. for 2 h. The resulting mixture was filtered, the filter cake was washed with EA (2×20 mL). The filtrate was extracted with EA (3×30 mL), the organic phases were combined and washed with brine (50 mL), dried by Na₂SO₄. The solid was filtered out, the filtrate was concentrated. This resulted in 3-(3,5-dimethyl-1,2-oxazol-4-yl)-4-methoxyaniline (5.60 g, 61.73%) as red oil. LC/MS: mass calcd. For C₁₂H₁₄N₂O₂: 218.11, found: 219.15 [M+H]⁺.

### Step 3: Synthesis of 1,3-diethyl 2-([[3-(3,5-dimethyl-1,2-oxazol-4-yl)-4-methoxyphenyl]amino]methylidene)propanedioate

Into a 250 mL flask was added 3-(3,5-dimethyl-1,2-oxazol-4-yl)-4-methoxyaniline (5.60 g, 25.66 mmol, 1.00 equiv), 1,3-diethyl 2-(ethoxymethylidene)propanedioate (5.60 g, 25.92 mmol, 1.01 equiv). The mixture was stirred at 130 degrees C. for 0.5 h. The reaction mixture was purified by flash chromatography, eluted with PE:EA=3:1 to afford 1,3-diethyl 2-([[3-(3,5-dimethyl-1,2-oxazol-4-yl)-4-methoxyphenyl]amino]methylidene)propanedioate (6.60 g, 63.71%) as orange solid. LC/MS: mass calcd. For C₂₀H₂₄N₂O₆: 388.16, found: 389.05 [M+H]⁺.

### Step 4: Synthesis of ethyl 7-(3,5-dimethyl-1,2-oxazol-4-yl)-4-hydroxy-6-methoxyquinoline-3-carboxylate

Into a 20 mL tube was added 1,3-diethyl 2-([[3-(3,5-dimethyl-1,2-oxazol-4-yl)-4-methoxyphenyl]amino]methylidene)propanedioate (3.60 g, 9.27 mmol, 1.00 equiv), diphenylether (14.00 mL), the reaction was stirred at 280 degrees C. for 15 mins by microwave. n-Hexane (15 mL) was added when the reaction temperature was down to 50 degrees C., some solid was appeared. When the mixture was cooled to room temperature, the supernatant liquid was removed, the residue was heated at 80 degrees C. in EA (20 mL) for 20 mins, then diluted with n-hexane (15 mL), cooled to room temperature. The solid was collected by filtration, washed with diethyl ether (15 mL) and dried under vacuum. This resulted in ethyl 7-(3,5-dimethyl-1,2-oxazol-4-yl)-4-hydroxy-6-methoxyquinoline-3-carboxylate (2.00 g, 47.27%) as brown solid. LC/MS: mass calcd. For C₁₈H₁₈N₂O₅: 342.12, found: 343.15 [M+H]⁺.

### Step 5: 7-(3,5-dimethyl-1,2-oxazol-4-yl)-6-methoxy-4-oxo-1H-quinoline-3-carboxylic acid

Into a 100 mL flask was added ethyl 7-(3,5-dimethyl-1,2-oxazol-4-yl)-4-hydroxy-6-methoxyquinoline-3-carboxylate (2.00 g, 5.842 mmol, 1.00 equiv), EtOH (9.00 mL), NaOH (2.0 M, 8.76 mL, 17.52 mmol, 3.00 equiv). The reaction was stirred at 80 degrees C. for 17 h. The reaction was concentrated, the residue was diluted with water (10 mL), the resulting solution was washed with EA (5 mL), the aqueous phase was acidified to pH=4 by 2M HCl. The solid was collected by filtration, washed with water (10 mL), dried under vacuum. This resulted in 7-(3,5-dimethyl-1,2-oxazol-4-yl)-6-methoxy-4-oxo-1H-quinoline-3-carboxylic acid (1.50 g, 57.19%) as brown solid. LC/MS: mass calcd. For C₁₆H₁₄N₂O₅: 314.09, found: 315.15 [M+H]⁺.

### Step 6: Synthesis of 4-chloro-7-(3,5-dimethyl-1,2-oxazol-4-yl)-6-methoxyquinoline-3-carboxamide

Into a 100 mL flask was added 7-(3,5-dimethyl-1,2-oxazol-4-yl)-6-methoxy-4-oxo-1H-quinoline-3-carboxylic acid (1.50 g, 4.77 mmol, 1.00 equiv), phosphorus oxychloride (20.00 mL), the reaction was stirred at 100 degrees C. for 2 h. The reaction was concentrated, the residue azeotroped with toluene (2×10 mL), the resulting dark brown gum was dissolved in THF (20.00 mL), added dropwise to NH₃.H₂O (20.00 mL) at 0 degrees C. The reaction was stirred at 0 degrees C. for 30 mins, the reaction was concentrated to half volume, diluted with water (10 mL) and the resulting dark brown solid collected by filtration. The solid was washed with water (10 mL) and dried under vacuum. This resulted in 4-chloro-7-(3,5-dimethyl-1,2-oxazol-4-yl)-6-methoxyquinoline-3-carboxamide (1.20 g, 66.82%) as black solid. LC/MS: mass calcd. For C₁₆H₁₄ClN₃O₃: 331.07, found: 332.10 [M+H]⁺.

### Step 7: Synthesis of 7-(3,5-dimethyl-1,2-oxazol-4-yl)-6-methoxy-4-[[(1R)-1-(pyridin-2-yl)ethyl]amino]quinolone-3-carboxamide

Into a 8 L sealed tube was added 4-chloro-7-(3,5-dimethyl-1,2-oxazol-4-yl)-6-methoxyquinoline-3-carboxamide (200.00 mg, 0.60 mmol, 1.00 equiv), NMP (5.00 mL), (1R)-1-(pyridin-2-yl)ethanamine (88.38 mg, 0.72 mmol, 1.20 equiv), DIEA (272.70 mg, 2.11 mmol, 3.50 equiv), the reaction was stirred at 150 degrees C. for 3 h under N₂ atmosphere by microwave. The reaction mixture was purified by reverse phase column with the following conditions: column, C18 silica gel; mobile phase, MeCN in water (0.05% NH₄HCO₃), 10% to 50% gradient in 30 min; detector, UV 254 nm. The fractions were combined and concentrated. This resulted in 7-(3,5-dimethyl-1,2-oxazol-4-yl)-6-methoxy-4-[[(1R)-1-(pyridin-2-yl)ethyl]amino]quinolone-3-carboxamide (140.00 mg, 52.62%) as yellow solid. LC/MS: mass calcd. For C₂₃H₂₃N₅O₃: 417.18, found: 418.20 [M+H]⁺.

### Step 8: Synthesis of 7-(3,5-dimethyl-1,2-oxazol-4-yl)-8-methoxy-1-[(1R)-1-(pyridin-2-yl)ethyl]-3H-imidazo[4,5-c]quinolin-2-one

Into a 8 mL flask was added 7-(3,5-dimethyl-1,2-oxazol-4-yl)-6-methoxy-4-[[(1R)-1-(pyridin-2-yl)ethyl]amino]quinoline-3-carboxamide (140.00 mg, 0.34 mmol, 1.00 equiv), MeOH (3.00 mL), KOH (24.46 mg, 0.44 mmol, 1.30 equiv), the mixture was cooled to 0 degrees C., PhI(OAc)₂ (129.58 mg, 0.40 mmol, 1.20 equiv) was added in batches. The reaction was stirred at 0 degrees C. for 1 h. The reaction was concentrated, the residue was purified by TLC-Plate with DCM:MeOH=10:1. This resulted in 7-(3,5-dimethyl-1,2-oxazol-4-yl)-8-methoxy-1-[(1R)-1-(pyridin-2-yl)ethyl]-3H-imidazo[4,5-c]quinolin-2-one (140.00 mg, 95.46%) as yellow oil. LC/MS: mass calcd. For C₂₃H₂₁N₅O₃: 415.16, found: 416.25 [M+H]⁺.

### Synthesis of (S)-1-(2-cyclopropyl-4-(2-(hydroxymethyl)benzyl)-6-(1,2,3,6-tetrahydropyridin-4-yl)-3,4-dihydroquinoxalin-1(2H)-yl)ethan-1-one (SM26)

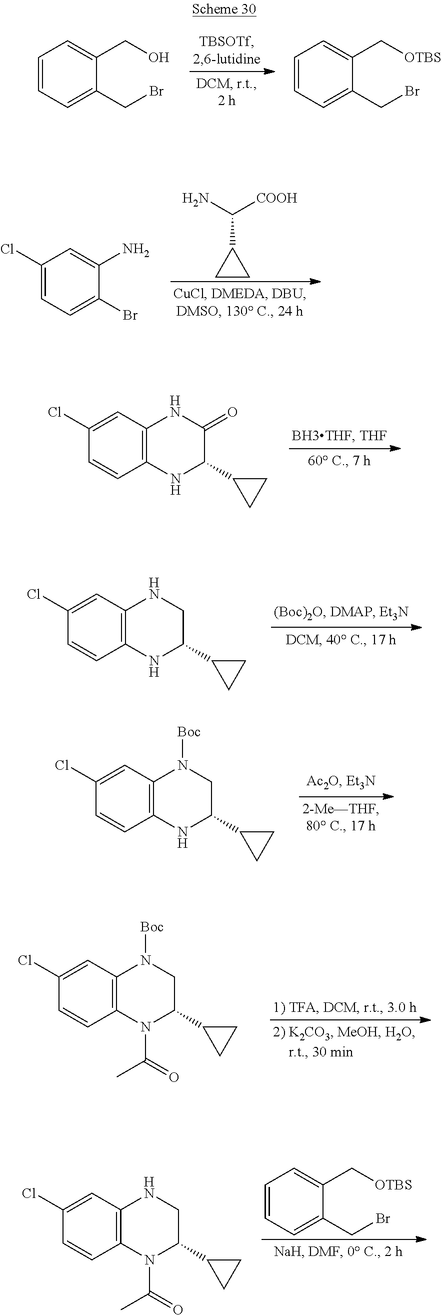

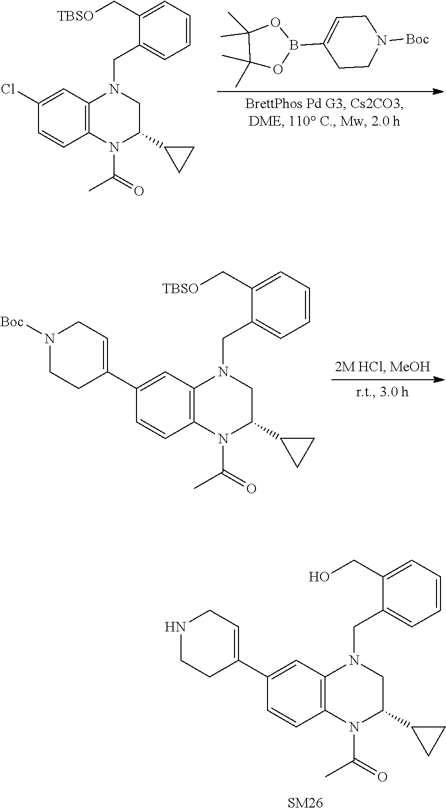

### Step 1: Synthesis of [[2-(bromomethyl)phenyl]methoxy](tert-butyl)dimethylsilane

To a stirred solution of [2-(bromomethyl)phenyl]methanol (2.00 g, 9.95 mmol, 1.00 equiv) in DCM (30.00 mL) was added 2,6-lutidine (2.32 mL, 21.62 mmol, 2.00 equiv) and TBSOTf (3.43 mL, 14.92 mmol, 1.50 equiv) dropwise at room temperature. The resulting mixture was stirred for 2 h at room temperature. Then it was diluted with H₂O (50 mL). The resulting mixture was extracted with DCM (3×100 mL). The combined organic layers were washed with brine (80 mL), dried over anhydrous Na₂SO₄.

After filtration, the filtrate was concentrated under reduced pressure. The residue was purified by silica gel column chromatography, eluted with PE:EA=10:1 to afford [[2-(bromomethyl)phenyl]methoxy](tert-butyl)dimethylsilane (2.40 g, 76.52%) as yellow oil. LC/MS: mass calcd. For C₁₄H₂₃BrOSi: 277.06, found: 222.05, 224.05 [M-OTBS+2+Na, M-OTBS+2+Na+2]⁺. ¹H NMR (400 MHz, CDCl₃) δ: 7.46-7.48 (m, 1H), 7.32-7.36 (m, 2H), 7.27-7.29 (m, 1H), 4.90 (s, 2H), 4.61 (s, 2H), 0.97 (s, 9H), 0.15 (s, 6H).

### Step 2: Synthesis of (3S)-7-chloro-3-cyclopropyl-3,4-dihydro-1H-quinoxalin-2-one

To a stirred solution of 2-bromo-5-chloroaniline (4.00 g, 19.37 mmol, 1.00 equiv) and (S)-amino(cyclopropyl)acetic acid (4.46 g, 38.74 mmol, 2.00 equiv) in DMSO (40.00 mL) were added CuCl (95.90 mg, 0.97 mmol, 0.05 equiv), DMEDA (341.56 mg, 3.88 mmol, 0.20 equiv) and DBU (5.90 g, 38.75 mmol, 2.00 equiv) in portions at room temperature. The resulting mixture was stirred for 24 h at 130 degrees C. under N₂ atmosphere. The resulting mixture was diluted with EA (80 mL). The resulting mixture was filtered, the filter cake was washed with EA (3×10 mL). The filtrate was concentrated under the reduced pressure. The residue was purified by reverse flash chromatography with the following conditions: column, C18 column; mobile phase, ACN in water (0.05% TFA), 30% to 45% gradient in 40 min; detector, UV254 nm. The fractions were combined and concentrated. (3S)-7-chloro-3-cyclopropyl-3,4-dihydro-1H-quinoxalin-2-one (2.60 g, 57.50%) was obtained as yellow solid. LC/MS: mass calcd. For C₁₁H₁₁ClN₂O: 222.06, found: 223.05 [M+H]⁺.

### Step 3: Synthesis of (2S)-6-chloro-2-cyclopropyl-1,2,3,4-tetrahydro-quinoxaline

To a stirred solution of (3S)-7-chloro-3-cyclopropyl-3,4-dihydro-1H-quinoxalin-2-one (2.60 g, 11.70 mmol, 1.00 equiv) in THF (50.00 mL) was added BH₃THF (1.0 M, 250.00 mL, 21.37 equiv) dropwise at room temperature. The resulting mixture was stirred for 7 h at 60 degrees C. under N₂ atmosphere. The reaction was quenched by the addition of MeOH (30 mL) and 1M HCl (30 mL), and stirred for 30 min at room temperature. The mixture was basified to pH 8˜10 with 2M NaOH. The resulting mixture was concentrated under reduced pressure. The resulting mixture was extracted with EA (3×100 mL). The combined organic layers were washed with brine (1×80 mL), dried over anhydrous Na₂SO₄. After filtration, the filtrate was concentrated under reduced pressure. The residue was purified by silica gel column chromatography, eluted with PE:EA=8:1 to afford (2S)-6-chloro-2-cyclopropyl-1,2,3,4-tetrahydro-quinoxaline (2.10 g, 86.30% yield) as yellow solid. LC/MS: mass calcd. For C₁₁H₁₃ClN₂: 208.08, found: 209.05 [M+H]⁺.

### Step 4: Synthesis of tert-butyl (3S)-7-chloro-3-cyclopropyl-3,4-dihydro-2H-quinoxaline-1-carboxylate

To a stirred solution of (2S)-6-chloro-2-cyclopropyl-1,2,3,4-tetrahydroquinoxaline (2.10 g, 10.06 mmol, 1.00 equiv) in DCM (40.00 mL) was added Et₃N (2.04 g, 20.13 mmol, 2.00 equiv), DMAP (0.61 g, 5.03 mmol, 0.50 equiv) and (Boc)₂O (2.64 g, 12.10 mmol, 1.20 equiv) in portions at room temperature. The resulting mixture was stirred for 17 h at 40 degrees C. The resulting mixture was concentrated under vacuum. The residue was purified by silica gel column chromatography, elute with PE:EA=25:1 to afford tert-butyl (3S)-7-chloro-3-cyclopropyl-3,4-dihydro-2H-quinoxaline-1-carboxylate (2.10 g, 55.46%) as yellow solid. LC/MS: mass calcd. For C₁₆H₂₁ClN₂O₂: 308.13, found: 309.15 [M+H]⁺.

### Step 5: Synthesis of tert-butyl (3S)-4-acetyl-7-chloro-3-cyclopropyl-2,3-dihydroquinoxaline-1-carboxylate

To a stirred solution of tert-butyl (3S)-7-chloro-3-cyclopropyl-3,4-dihydro-2H-quinoxaline-1-carboxylate (2.10 g, 6.80 mmol, 1.00 equiv) in 2-Methyltetrahydrofuran (20.00 mL) was added Et₃N (11.34 mL, 112.01 mmol, 12.00 equiv) and Ac₂O (6.34 mL, 68.02 mmol, 10.00 equiv) in portions at room temperature. The resulting mixture was stirred for 17 h at 80 degrees C. The mixture was allowed to cool down to room temperature. The resulting mixture was diluted with EA (60 mL), washed with 1 M HCl (3×40 mL), sat. NaHCO₃ aq. (3×40 mL) and brine (1×40 mL), dried over anhydrous Na₂SO₄. After filtration, the filtrate was concentrated under reduced pressure. Tert-butyl (3S)-4-acetyl-7-chloro-3-cyclopropyl-2,3-dihydroquinoxaline-1-carboxylate (2.90 g, crude) was obtained as red oil. LC/MS: mass calcd. For C₁₈H₂₃ClN₂O₃: 350.14, found: 373.20 [M+Na]⁺.

### Step 6: Synthesis of 1-[(2S)-6-chloro-2-cyclopropyl-3,4-dihydro-2H-quinoxalin-1-yl]ethanone

To a stirred solution of tert-butyl (3S)-4-acetyl-7-chloro-3-cyclopropyl-2,3-dihydroquinoxaline-1-carboxylate (2.90 g, 8.27 mmol, 1.00 equiv) in DCM (30.00 mL) was added TFA (10.00 mL) dropwise. The resulting mixture was stirred for 3 h at room temperature. Then the resulting mixture was concentrated under vacuum. The residue was dissolved in MeOH (30.00 mL). To the above mixture was added K₂CO₃ (3.43 g, 24.82 mmol, 3.00 equiv) in H₂O (15.00 mL) dropwise at room temperature. The resulting mixture was stirred for additional 30 mins at room temperature. The resulting mixture was concentrated under vacuum. The residue was dissolved in H₂O (30 mL). The resulting mixture was extracted with EA (3×50 mL). The combined organic layers were washed with brine (50 mL), dried over anhydrous Na₂SO₄. After filtration, the filtrate was concentrated under vacuum. 1-[(2S)-6-chloro-2-cyclopropyl-3,4-dihydro-2H-quinoxalin-1-yl]ethanone (1.70 g, crude) was obtained as yellow oil. LC/MS: mass calcd. For C₁₃H₁₅ClN₂O: 250.09, found: 251.15 [M+H]⁺.

### Step 7: Synthesis of 1-[(2S)-4-[(2-[[(tert-butyldimethylsilyl)oxy]methyl]phenyl)methyl]-6-chloro-2-cyclopropyl-2,3-dihydroquinoxalin-1-yl]ethanone

To a stirred solution of 1-[(2S)-6-chloro-2-cyclopropyl-3,4-dihydro-2H-quinoxalin-1-yl]ethanone (700.00 mg, 2.79 mmol, 1.00 equiv) in DMF (10.00 mL) was added NaH (60%, 200.99 mg, 8.38 mmol, 3.00 equiv) in portions at 0 degrees C. under N₂ atmosphere. The resulting mixture was stirred for 30 min at 0 degrees C. under N₂ atmosphere. To the above mixture was added [[2-(bromomethyl)phenyl]methoxy](tert-butyl)dimethylsilane (1.10 g, 3.488 mmol, 1.25 equiv) in DMF (5.00 mL) dropwise at 0 degrees C. The resulting mixture was stirred for additional 1.5 h at 0 degrees C. The resulting mixture was diluted with H₂O (45 mL). The resulting mixture was extracted with EA (3×80 mL). The combined organic layers were washed with brine (3×120 mL), dried over anhydrous Na₂SO₄. After filtration, the filtrate was concentrated under vacuum. The residue was purified by silica gel column chromatography, eluted with PE:EA=8:1 to afford 1-[(2S)-4-[(2-[[(tert-butyldimethylsilyl)oxy]methyl]phenyl)methyl]-6-chloro-2-cyclopropyl-2,3-dihydroquinoxalin-1-yl]ethanone (1.10 g, 77.90%) as yellow solid. LC/MS: mass calcd. For C₂₇H₃₇ClN₂O₂Si: 484.23, found: 485.30 [M+H]⁺.

### Step 8: Synthesis of tert-butyl 4-[(2S)-1-acetyl-4-[(2-[[(tert-butyldimethylsilyl)oxy]methyl]phenyl)methyl]-2-cyclopropyl-2,3-dihydroquinoxalin-6-yl]-3,6-dihydro-2H-pyridine-1-carboxylate

To a stirred solution of 1-[(2S)-4-[(2-[[(tert-butyldimethylsilyl)oxy]methyl]phenyl)methyl]-6-chloro-2-cyclopropyl-2,3-dihydroquinoxalin-1-yl]ethanone (400.00 mg, 0.83 mmol, 1.00 equiv) in DME (12.00 mL) was added tert-butyl 4-(4,4,5,5-tetramethyl-1,3,2-dioxaborolan-2-yl)-3,6-dihydro-2H-pyridine-1-carboxylate (509.89 mg, 1.65 mmol, 2.00 equiv), Cs₂CO₃ (805.92 mg, 2.47 mmol, 3.00 equiv) and BrettPhos Pd G3 (74.74 mg, 0.08 mmol, 0.10 equiv) in portions at room temperature. The final reaction mixture was irradiated with microwave radiation for 2 h at 110 degrees C. under N₂ atmosphere. The resulting mixture was concentrated under vacuum. The residue was purified by silica gel column chromatography, eluted with PE:EA=4:1 to afford tert-butyl 4-[(2S)-1-acetyl-4-[(2-[[(tert-butyldimethylsilyl)oxy]methyl]phenyl)methyl]-2-cyclopropyl-2,3-dihydroquinoxalin-6-yl]-3,6-dihydro-2H-pyridine-1-carboxylate (450.00 mg, 75.62%) as yellow solid. LC/MS: mass calcd. For C₃₋₇H₅₃N₃O₄Si: 631.38, found: 632.55 [M+H]⁺.

### Step 9: Synthesis of 1-[(2S)-2-cyclopropyl-4-[[2-(hydroxymethyl)phenyl]methyl]-6-(1,2,3,6-tetrahydropyridin-4-yl)-2,3-dihydroquinoxalin-1-yl]ethanone (SM26)

To a stirred solution of tert-butyl 4-[(2S)-1-acetyl-4-[(2-[[(tert-butyldimethylsilyl)oxy]methyl]phenyl)methyl]-2-cyclopropyl-2,3-dihydroquinoxalin-6-yl]-3,6-dihydro-2H-pyridine-1-carboxylate (430.00 mg, 0.68 mmol, 1.00 equiv) in MeOH (10.00 mL) was added 2M HCl (10.00 mL) dropwise at room temperature. The resulting mixture was stirred for 3 h at room temperature. The resulting mixture was concentrated under vacuum. 1-[(2S)-2-cyclopropyl-4-[[2-(hydroxymethyl)phenyl]methyl]-6-(1,2,3,6-tetrahydropyridin-4-yl)-2,3-dihydroquinoxalin-1-yl]ethanone (340.00 mg, crude) was obtained as yellow solid. LC/MS: mass calcd. For C₂₆H₃₁N₃O₂: 417.24, found: 418.30 [M+H]⁺.

### Synthesis of (S)-5-(1-acetyl-2-cyclopropyl-4-(2-(hydroxymethyl)benzyl)-1,2,3,4-tetrahydroquinoxalin-6-yl)pyrimidine-2-carboxylic acid (SM27)

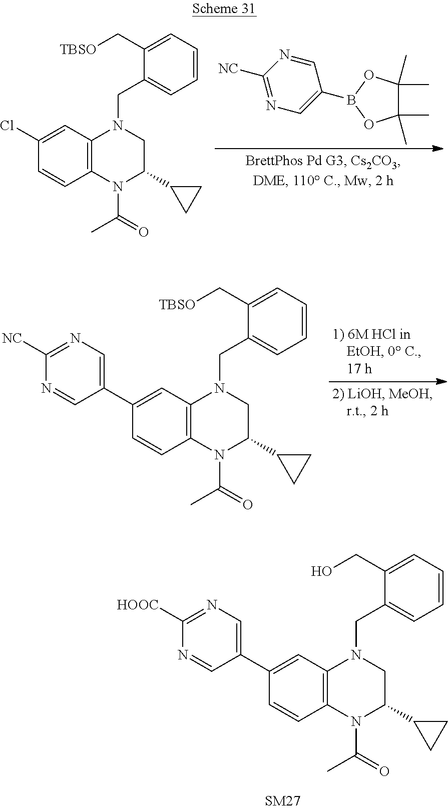

### Step 1: Synthesis of (S)-5-(1-acetyl-4-(2-(((tert-butyldimethylsilyl)oxy)methyl)benzyl)-2-cyclopropyl-1,2,3,4-tetrahydroquinoxalin-6-yl)pyrimidine-2-carbonitrile

To a stirred solution of 1-[(2S)-4-[(2-[[(tert-butyldimethylsilyl)oxy]methyl]phenyl)methyl]-6-chloro-2-cyclopropyl-2,3-dihydroquinoxalin-1-yl]ethanone (600.00 mg, 1.24 mmol, 1.00 equiv) in DME (12.00 ml) was added 5-(4,4,5,5-tetramethyl-1,3,2-dioxaborolan-2-yl)pyrimidine-2-carbonitrile (571.53 mg, 2.47 mmol, 2.00 equiv), Cs₂CO₃ (1.20 g, 3.71 mmol, 3.00 equiv) and BrettPhos Pd G3 (112.11 mg, 0.12 mmol, 0.10 equiv) in portions at room temperature. The final reaction mixture was irradiated with microwave radiation for 2 h at 110 degrees C. under N₂ atmosphere. The resulting mixture was concentrated under vacuum. The residue was purified by silica gel column chromatography, eluted with PE:EA=3:1 to afford 5-[(2S)-1-acetyl-4-[(2-[[(tert-butyldimethylsilyl)oxy]methyl]phenyl)methyl]-2-cyclopropyl-2,3-dihydroquinoxalin-6-yl]pyrimidine-2-carbonitrile (550.00 mg, 80.30%) as a yellow oil. LC/MS: mass calcd. For C₃₂H₃₉N₅O₂Si: 553.29, found: 554.40 [M+H]⁺.

### Step 2: Synthesis of (S)-5-(1-acetyl-2-cyclopropyl-4-(2-(hydroxymethyl)benzyl)-1,2,3,4-tetrahydroquinoxalin-6-yl)pyrimidine-2-carboxylic acid (SM27)

A solution of 5-[(2S)-1-acetyl-4-[(2-[[(tert-butyldimethylsilyl)oxy]methyl]phenyl)methyl]-2-cyclopropyl-2,3-dihydroquinoxalin-6-yl]pyrimidine-2-carbonitrile (400.00 mg, 0.72 mmol, 1.00 equiv) in HCl in EtOH (38%) (10.00 mL) was stirred 17 h at 0 degrees C. The resulting mixture was concentrated under vacuum. The residue was dissolved in MeOH (8.00 mL). To the above mixture was added LiOH.H₂O (90.93 mg, 2.17 mmol, 3.00 equiv) in H₂O (4.00 mL) dropwise at room temperature. The resulting mixture was stirred for additional 2 h at room temperature. The resulting mixture was concentrated under vacuum. The residue was dissolved in H₂O (4 mL). The mixture was acidified to pH 3˜5 with 2M HCl. The precipitated solid were collected by filtration and washed with H₂O (3×5 mL), dried under vacuum. The solid was purified by reverse flash chromatography with the following conditions: column, C18 column; mobile phase, ACN in water (0.05% TFA), 15% to 30% gradient in 20 min; detector, UV 254 nm. The fractions were combined and concentrated. 5-[(2S)-1-acetyl-2-cyclopropyl-4-[[2-(hydroxymethyl)phenyl]methyl]-2,3-dihydroquinoxalin-6-yl] pyrimidine-2-carboxylic acid (140.00 mg, 40.81%) was obtained as yellow oil. LC/MS: mass calcd. For C₂₆H₂₆N₄O₄: 458.20, found: 459.30 [M+H]⁺.

### Example 1C: Synthesis of Protein-Binding Moiety Intermediate Derivatives

### Synthesis of (S)-2-(4-(4-chlorophenyl)-2,3,9-trimethyl-6H-thieno[3,2-f][1,2,4]triazolo[4,3-a][1,4]diazepin-6-yl)-N-(4-(piperazin-1-yl)phenyl)acetamide

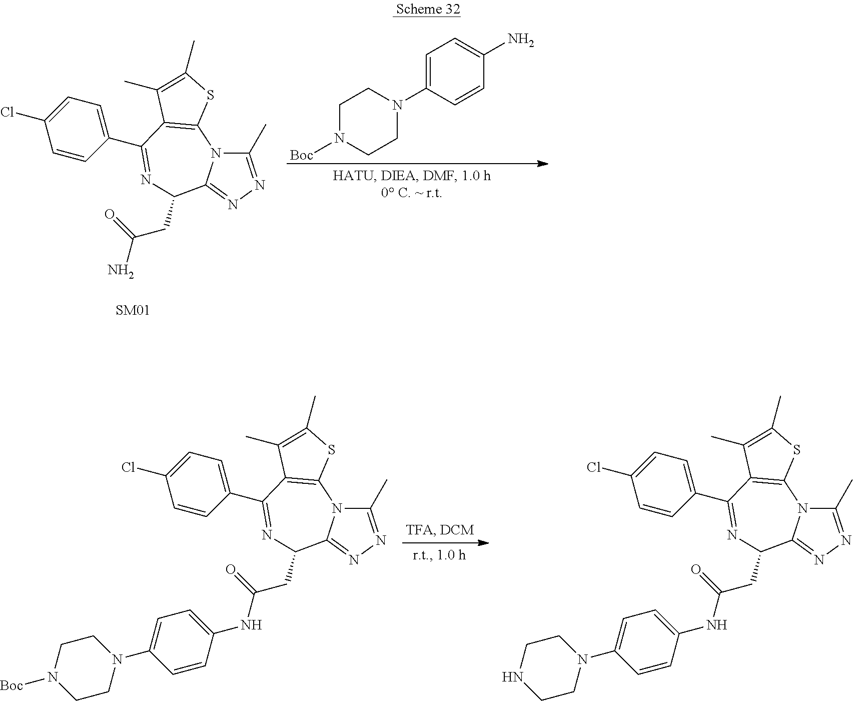 [SMILES: Cc1sc2c(c1C)C(c1ccc(Cl)cc1)=N[C@@H](CC(N)=O)c1nnc(C)n1-2 ; CC(C)(C)OC(=O)N1CCN(c2ccc(N)cc2)CC1 ; Cc1sc2c(c1C)C(c1ccc(Cl)cc1)=N[C@@H](CC(=O)Nc1ccc(N3CCN(C(=O)OC(C)(C)C)CC3)cc1)c1nnc(C)n1-2 ; Cc1sc2c(c1C)C(c1ccc(Cl)cc1)=N[C@@H](CC(=O)Nc1ccc(N3CCNCC3)cc1)c1nnc(C)n1-2]

### Step 1: Synthesis of tert-butyl (S)-4-(4-(2-(4-(4-chlorophenyl)-2,3,9-trimethyl-6H-thieno[3,2-f][1,2,4]triazolo[4,3-a][1,4]diazepin-6-yl)acetamido)phenyl)piperazine-1-carboxylate

To a stirred solution of (S)-2-(4-(4-chlorophenyl)-2,3,9-trimethyl-6H-thieno[3,2-f][1,2,4]triazolo[4,3-a][1,4]diazepin-6-yl)acetic acid (150.00 mg, 0.37 mmol, 1.00 equiv) in DMF (3.50 mL) was added HATU (170.73 mg, 0.45 mmol, 1.20 equiv), DIEA (145.08 mg, 1.12 mmol, 3.00 equiv) and tert-butyl 4-(4-aminophenyl) piperazine-1-carboxylate (114.16 mg, 0.41 mmol, 1.10 equiv) in portions at 0 degrees C. The resulting mixture was stirred for 1 h at room temperature. The reaction mixture was poured into ice/water (10 mL), the solid was filtered out and washed by water (3 mL), dried under vacuum. Tert-butyl (S)-4-(4-(2-(4-(4-chlorophenyl)-2,3,9-trimethyl-6H-thieno[3,2-f][1,2,4]triazolo[4,3-a][1,4]diazepin-6-yl)acetamido)phenyl)piperazine-1-carboxylate (240.00 mg, 87.43%) was obtained as pure solid. LC/MS: mass calcd. For C₃₄H₃₈ClN₇O₃S: 659.24, found: 660.10 [M+H]⁺.

### Step 2: Synthesis of (S)-2-(4-(4-chlorophenyl)-2,3,9-trimethyl-6H-thieno[3,2-f][1,2,4]triazolo[4,3-a][1,4]diazepin-6-yl)-N-(4-(piperazin-1-yl)phenyl)acetamide

To a stirred solution of tert-butyl (S)-4-(4-(2-(4-(4-chlorophenyl)-2,3,9-trimethyl-6H-thieno[3,2-f][1,2,4]triazolo[4,3-a][1,4]diazepin-6-yl)acetamido)phenyl)piperazine-1-carboxylate (220.00 mg, 0.33 mmol, 1.00 equiv) in DCM (4.00 mL) was added TFA (0.80 mL) dropwise at room temperature. The resulting mixture was stirred for 1 h at room temperature. The resulting mixture was concentrated under vacuum. (S)-2-(4-(4-chlorophenyl)-2,3,9-trimethyl-6H-thieno[3,2-f][1,2,4]triazolo[4,3-a][1,4]diazepin-6-yl)-N-(4-(piperazin-1-yl)phenyl)acetamide (220.00 mg, crude) was obtained as yellow oil. LC/MS: mass calcd. For C₂₉H₃₀ClN₇OS: 559.19, found: 560.45 [M+H]⁺.

### Synthesis of 2-((S)-4-(4-chlorophenyl)-2,3,9-trimethyl-6H-thieno[3,2-f][1,2,4]triazolo[4,3-a][1,4]diazepin-6-yl)-N-((1r,4S)-4-(methylamino)cyclohexyl)acetamide and 2-((S)-4-(4-chlorophenyl)-2,3,9-trimethyl-6H-thieno[3,2-f][1,2,4]triazolo[4,3-a][1,4]diazepin-6-yl)-N-((1s,4R)-4-(methylamino)cyclohexyl)acetamide

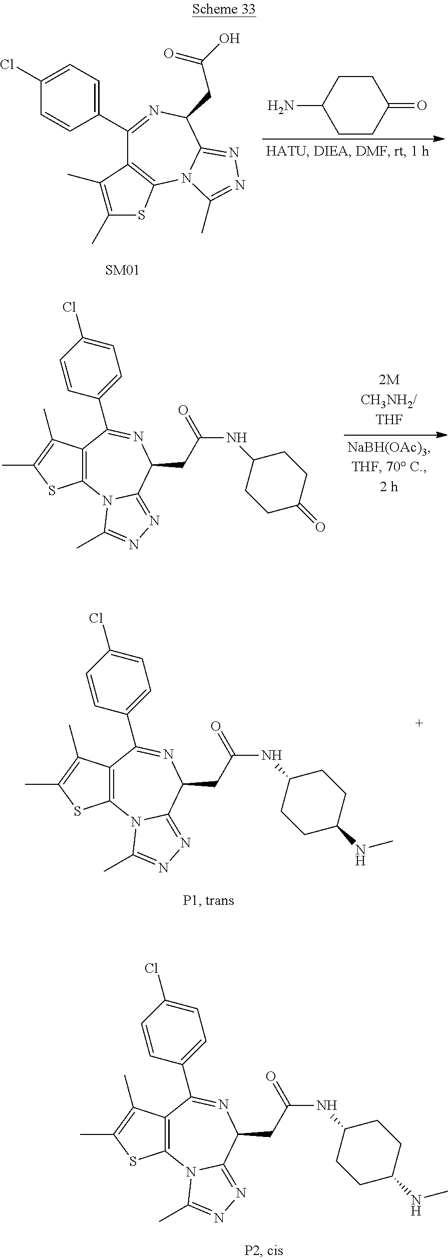

### Step 1: Synthesis of (S)-2-(4-(4-chlorophenyl)-2,3,9-trimethyl-6H-thieno[3,2-f][1,2,4]triazolo[4,3-a][1,4]diazepin-6-yl)-N-(4-oxocyclohexyl)acetamide

The procedure was the same as tert-butyl (S)-4-(4-(2-(4-(4-chlorophenyl)-2,3,9-trimethyl-6H-thieno[3,2-f][1,2,4]triazolo[4,3-a][1,4]diazepin-6-yl)acetamido)phenyl)piperazine-1-carboxylate. 500.00 mg of (S)-2-(4-(4-chlorophenyl)-2,3,9-trimethyl-6H-thieno[3,2-f][1,2,4]triazolo[4,3-a][1,4]diazepin-6-yl)acetic acid was used, 600.00 mg of (S)-2-(4-(4-chlorophenyl)-2,3,9-trimethyl-6H-thieno[3,2-f][1,2,4]triazolo[4,3-a][1,4]diazepin-6-yl)-N-(4-oxocyclohexyl)acetamide was obtained as light yellow solid (92.13% yield). LC/MS: mass calcd. For C₂₅H₂₆ClN₅O₂S: 495.15, found: 496.15 [M+H]⁺.

### Step 2: Synthesis of 2-((S)-4-(4-chlorophenyl)-2,3,9-trimethyl-6H-thieno[3,2-f][1,2,4]triazolo[4,3-a][1,4]diazepin-6-yl)-N-((1r,4S)-4-(methylamino)cyclohexyl)acetamide and 2-((S)-4-(4-chlorophenyl)-2,3,9-trimethyl-6H-thieno[3,2-f][1,2,4]triazolo[4,3-a][1,4]diazepin-6-yl)-N-((1s,4R)-4-(methylamino)cyclohexyl)acetamide

To a stirred solution of (S)-2-(4-(4-chlorophenyl)-2,3,9-trimethyl-6H-thieno[3,2-f][1,2,4]triazolo[4,3-a][1,4]diazepin-6-yl)-N-(4-oxocyclohexyl)acetamide (620.00 mg, 1.25 mmol, 1.00 equiv) and Methylamine (2M in THF, 6.25 mL, 12.50 mmol, 10.00 equiv) in tetrahydrofuran (10.00 mL) were added sodium triacetoxyborohydride (317.89 mg, 1.50 mmol, 1.20 equiv) at room temperature. The resulting mixture was stirred for 2.0 h at 70 degrees C. After reaction, the reaction was quenched with sat. NH₄Cl (10 mL) at room temperature. The resulting mixture was extracted with EtOAc (3×10 mL). The combined organic layers were washed with water (3×5 mL), dried over anhydrous Na₂SO₄. After filtration, the filtrate was concentrated under reduced pressure. The residue was dissolved in DMF (3.0 mL). The residue was filtered and the filtration in DMF (3.0 mL) was purified by Perp-HPLC with the following conditions: Column: YMC-Actus Triart C18, 30 mm×150 mm, 5 um; Mobile Phase A: Water (10 MMOL/L NH₄HCO₃+0.1% NH₃.H2O), Mobile Phase B: ACN; Flow rate: 60 mL/min; Gradient: 27 B to 43 B in 9 min, 254 nm; RT1: 6,6.73. The fractions were combined and lyophilized directly. This resulted in 2-((S)-4-(4-chlorophenyl)-2,3,9-trimethyl-6H-thieno[3,2-f][1,2,4]triazolo[4,3-a][1,4]diazepin-6-yl)-N-((1r,4S)-4-(methylamino)cyclohexyl)acetamide (DT188-150-P1) (230.00 mg, 34.92%) as light yellow oil and 2-((S)-4-(4-chlorophenyl)-2,3,9-trimethyl-6H-thieno[3,2-f][1,2,4]triazolo[4,3-a][1,4]diazepin-6-yl)-N-((1s,4R)-4-(methylamino)cyclohexyl)acetamide (DT188-150-P2) (183.00 mg, 26.64%) as light yellow oil. LC/MS: mass calcd. For C₂₆H₃₁ClN₆OS: 510.20, found: 511.15 [M+H]⁺.

¹H NMR of DT188-150-P1: ¹H NMR (400 MHz, CD₃OD) δ: 7.42-7.48 (m, 4H), 4.68-4.71 (m, 1H), 3.68-3.78 (m, 1H), 3.38-3.44 (m, 1H), 3.28-3.33 (m, 1H), 3.01-3.09 (m, 1H), 2.75 (s, 3H), 2.71 (s, 3H), 2.46 (s, 3H), 2.09-2.16 (m, 4H), 1.71 (s, 3H), 1.43-1.48 (m, 4H). HPLC of DT188-150-P1: rt=3.425.

¹H NMR of DT188-150-P2: ¹H NMR (400 MHz, CD₃OD) δ: 7.42-7.47 (m, 4H), 4.70 (t, J=7.2 Hz, 1H), 3.95-4.05 (m, 1H), 3.35-3.47 (m, 2H), 3.08-3.15 (m, 1H), 2.75 (s, 3H), 2.72 (s, 3H), 2.46 (s, 3H), 1.92-2.00 (m, 4H), 1.72-1.82 (m, 3H), 1.70 (s, 3H), 1.67-1.72 (m, 1H). HPLC of DT188-150-P2: rt=3.528.

Structure Confirmation for Synthetic Intermediate

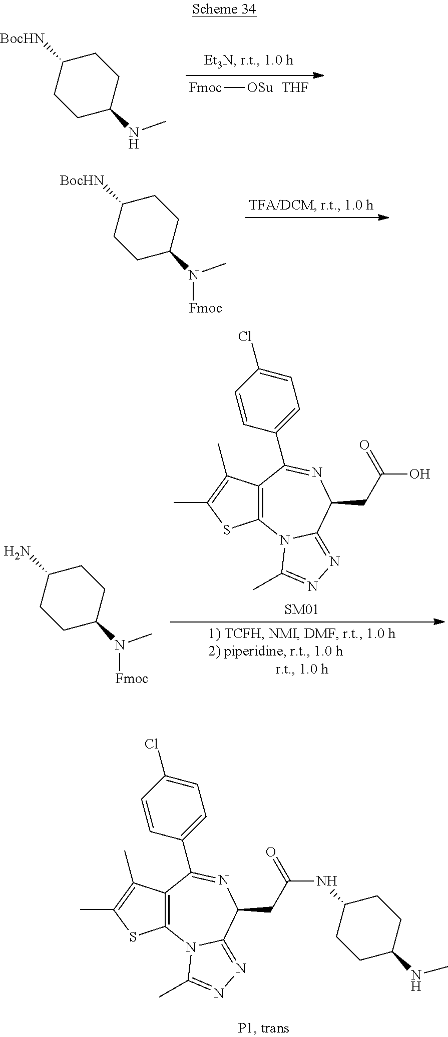

### Step 1: Synthesis of 9H-fluoren-9-ylmethyl N-methyl-N-[(1r,4r)-4-[(tert-butoxycarbonyl)amino] cyclohexyl]carbamate

To a stirred solution of tert-butyl N-[(1r,4r)-4-(methylamino)cyclohexyl]carbamate (500.00 mg, 2.19 mmol, 1.00 equiv) and 2,5-dioxopyrrolidin-1-yl 9H-fluoren-9-ylmethyl carbonate (738.67 mg, 2.19 mmol, 1.00 equiv) in tetrahydrofuran (10.00 mL) was added Et₃N (663.57 mg, 6.57 mmol, 3.00 equiv) dropwise at 0 degrees C. The resulting mixture was stirred for 1 h at room temperature. After reaction, the reaction was quenched with water (10 mL) at 0 degrees C. The resulting mixture was extracted with EtOAc (3×10 mL). The combined organic layers were washed with water (3×5 mL), dried over anhydrous Na₂SO₄. After filtration, the filtrate was concentrated under reduced pressure. The residue was purified by silica gel column chromatography, eluted with PE/EA (3:1) to afford 9H-fluoren-9-ylmethyl N-methyl-N-[(1r,4r)-4-[(tert-butoxycarbonyl)amino] cyclohexyl]carbamate (830.00 mg, 74.87%) as light yellow oil. LC/MS: mass calcd. For C₂₇H₃₄N₂O₄: 450.25, found: 473.20 [M+Na]⁺.

### Step 2: Synthesis of 9H-fluoren-9-ylmethyl N-methyl-N-[(1r,4r)-4-aminocyclohexyl]carbamate

To a stirred solution of 9H-fluoren-9-ylmethyl N-methyl-N-[(1r,4r)-4-[(tert-butoxycarbonyl)amino]cyclohexyl]carbamate (20.00 mg) in DCM (1.00 mL) was added TFA (0.25 mL) dropwise at 0 degrees C. The resulting mixture was stirred for 1 h at room temperature. After reaction, the resulting mixture was concentrated under reduced pressure. This resulted in 9H-fluoren-9-ylmethyl N-methyl-N-[(1r,4r)-4-aminocyclohexyl]carbamate (18.00 mg crude) as light yellow oil. The crude product was used in the next step directly without further purification. LC/MS: mass calcd. For C₂₂H₂₆N₂O₂: 350.20, found: 351.30 [M+H]⁺.

### Step 3: Synthesis of 2-((S)-4-(4-chlorophenyl)-2,3,9-trimethyl-6H-thieno[3,2-f][1,2,4]triazolo[4,3-a][1,4]diazepin-6-yl)-N-((1r,4S)-4-(methylamino)cyclohexyl)acetamide

To a stirred solution of (S)-2-(4-(4-chlorophenyl)-2,3,9-trimethyl-6H-thieno [3,2-f][1,2,4]triazolo[4,3-a][1,4]diazepin-6-yl)acetic acid (20.59 mg, 0.05 mmol, 1.00 equiv) in dimethylformamide (1.00 mL) was added NMI (21.08 mg, 0.255 mmol, 5.00 equiv), TCFH (28.82 mg, 0.102 mmol, 2.00 equiv) and 9H-fluoren-9-ylmethyl N-methyl-N-[(1r,4r)-4-aminocyclohexyl]carbamate (17.50 mg, 0.05 mmol, 1.00 equiv) at 0 degrees C. The resulting mixture was stirred for 1 h at room temperature. Then piperidine (0.10 mL) was added dropwise to the mixture at room temperature. The resulting mixture was stirred for 1 h at room temperature. After reaction, the reaction mixture was filtered and the filtration in DMF (2.0 mL) was purified by Perp-HPLC with the following conditions: Column: YMC-Actus Triart C18, 30 mm×150 mm, 5 μm; Mobile Phase A: Water(0.05% TFA), Mobile Phase B: ACN; Flow rate: 60 mL/min; Gradient: 27 B to 43 B in 9 min; 254 nm; RT1: 7.42. The fractions were combined and lyophilized directly. This resulted in 2-((S)-4-(4-chlorophenyl)-2,3,9-trimethyl-6H-thieno[3,2-f][1,2,4]triazolo[4,3-a][1,4]diazepin-6-yl)-N-((1r,4S)-4-(methylamino)cyclohexyl)acetamide (10 mg, 39.22%) as light yellow oil. LC/MS: mass calcd. For C₂₆H₃₁ClN₆OS: 510.20, found: 511.20 [M+H]⁺. HPLC: rt=3.433. ¹H NMR (400 MHz, CD₃OD) δ: 7.42-7.48 (m, 4H), 4.68-4.68 (m, 1H), 3.72-3.78 (m, 1H), 3.38-3.45 (m, 1H), 3.28-3.30 (m, 1H), 3.05-3.09 (m, 1H), 2.73 (s, 6H), 2.47 (s, 3H), 2.05-2.22 (m, 4H), 1.72 (s, 3H), 1.45-1.54 (m, 4H).

### Synthesis of (S)-2-(4-(4-chlorophenyl)-2,3,9-trimethyl-6H-thieno[3,2-f][1,2,4]triazolo[4,3-a][1,4]diazepin-6-yl)-N-(3-hydroxyphenyl)acetamide

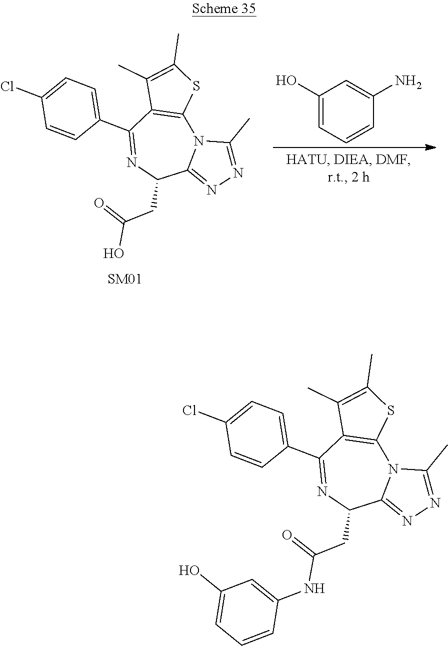 [SMILES: Cc1sc2c(c1C)C(c1ccc(Cl)cc1)=N[C@@H](CC(=O)O)c1nnc(C)n1-2 ; Nc1cccc(O)c1 ; Cc1sc2c(c1C)C(c1ccc(Cl)cc1)=N[C@@H](CC(=O)Nc1cccc(O)c1)c1nnc(C)n1-2]

The procedure was the same as tert-butyl (S)-4-(4-(2-(4-(4-chlorophenyl)-2,3,9-trimethyl-6H-thieno[3,2-f][1,2,4]triazolo[4,3-a][1,4]diazepin-6-yl)acetamido)phenyl)piperazine-1-carboxylate. 300.00 mg of (S)-2-(4-(4-chlorophenyl)-2,3,9-trimethyl-6H-thieno[3,2-f][1,2,4]triazolo[4,3-a][1,4]diazepin-6-yl)acetic acid was used, 250.00 mg of (S)-2-(4-(4-chlorophenyl)-2,3,9-trimethyl-6H-thieno[3,2-f][1,2,4]triazolo[4,3-a][1,4]diazepin-6-yl)-N-(3-hydroxyphenyl)acetamide was obtained as white solid (57.72% yield). LC/MS: mass calcd. For C₂₅H₂₂ClN₅O₂S: 491.12, found: 492.25 [M+1]⁺.

### Synthesis of (S)-2-(4-(4-chlorophenyl)-2,3,9-trimethyl-6H-thieno[3,2-f][1,2,4]triazolo[4,3-a][1,4]diazepin-6-yl)-N-(5-hydroxypyridin-3-yl)acetamide

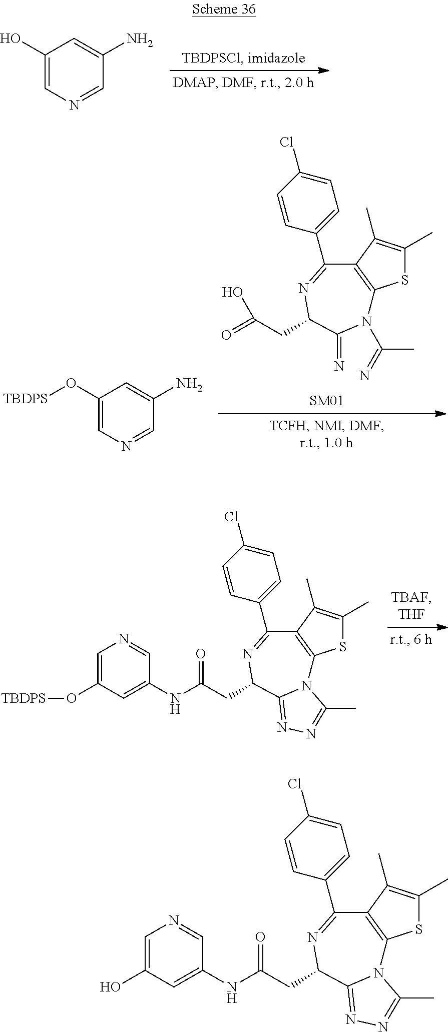

### Step 1: Synthesis of 5-[(tert-butyldiphenylsilyl)oxy]pyridin-3-amine

To a stirred solution of 5-aminopyridin-3-ol (200.00 mg, 1.82 mmol, 1.00 equiv) and DMAP (443.78 mg, 3.63 mmol, 2.00 equiv) in DMF (10.00 mL) were added imidazole (12.36 mg, 0.18 mmol, 0.10 equiv) and TBDPSCl (549.14 mg, 2.00 mmol, 1.10 equiv) at 0 degrees C. The solution was stirred for 2 h at room temperature. The reaction mixture was poured into 30 mL ice/water. The resulting mixture was extracted with EA (3×20 mL). The combined organic layers were washed with water (50 mL), dried over anhydrous Na₂SO₄. After filtration, the filtrate was concentrated under reduced pressure. The residue was purified by TLC plate with PE:EA=2:1 to afford 5-[(tert-butyldiphenylsilyl)oxy]pyridin-3-amine (230.5 mg, 43.70%) as yellow oil. LC/MS: mass calcd. For C₂₁H₂₄N₂OSi: 348.17, found: 349.20 [M+H]⁺.

### Step 2: Synthesis of (S)—N-(5-((tert-butyldiphenylsilyl)oxy)pyridin-3-yl)-2-(4-(4-chlorophenyl)-2,3,9-trimethyl-6H-thieno[3,2-f][1,2,4]triazolo[4,3-a][1,4]diazepin-6-yl)acetamide

To a stirred solution of (S)-2-(4-(4-chlorophenyl)-2,3,9-trimethyl-6H-thieno[3,2-f][1,2,4]triazolo[4,3-a][1,4]diazepin-6-yl)acetic acid (220.00 mg, 0.55 mmol, 1.00 equiv) and NMI (135.17 mg, 1.65 mmol, 3.00 equiv) in DMF (5.00 mL) were added TCFH (461.94 mg, 1.65 mmol, 3.00 equiv) and 5-[(tert-butyldiphenylsilyl)oxy] pyridin-3-amine (191.27 mg, 0.55 mmol, 1.00 equiv) at 0 degrees C. The resulting mixture was stirred for 1 h at room temperature. The reaction was poured into 15 mL ice-water. The solid was filtered out and wash by water (3×2 mL). The solid was dried under vacuum. This resulted in (S)—N-(5-((tert-butyldiphenylsilyl)oxy)pyridin-3-yl)-2-(4-(4-chlorophenyl)-2,3,9-trimethyl-6H-thieno[3,2-f][1,2,4]triazolo[4,3-a][1,4]diazepin-6-yl)acetamide (360.00 mg, 80.72%) as yellow solid. LC/MS: mass calcd. For C₄₀H₃₉ClN₆O₂SSi: 730.23, found: 493.15 [M-TBDPS+H]⁺.

### Step 3: Synthesis of (S)-2-(4-(4-chlorophenyl)-2,3,9-trimethyl-6H-thieno[3,2-f][1,2,4]triazolo[4,3-a][1,4]diazepin-6-yl)-N-(5-hydroxypyridin-3-yl)acetamide

To a stirred solution (S)—N-(5-((tert-butyldiphenylsilyl)oxy)pyridin-3-yl)-2-(4-(4-chlorophenyl)-2,3,9-trimethyl-6H-thieno[3,2-f][1,2,4]triazolo[4,3-a][1,4]diazepin-6-yl)acetamide (350.00 mg, 0.48 mmol, 1.00 equiv) in THF (2.00 mL) was added TBAF (1M in THF, 0.50 mL) dropwise at 0 degrees C. Then the solution was stirred for 6 h at room temperature. 10.0 mL H₂O was added to the mixture, the resulting mixture was extracted with EA (3×10 mL). The combined organic layers were washed with H₂O (10 mL), dried over anhydrous Na₂SO₄. After filtration, the filtrate was concentrated under reduced pressure. And the residue was purified by silica gel column chromatography, eluted with CH₂Cl₂/MeOH (10:01) to afford (S)-2-(4-(4-chlorophenyl)-2,3,9-trimethyl-6H-thieno[3,2-f][1,2,4]triazolo[4,3-a][1,4]diazepin-6-yl)-N-(5-hydroxypyridin-3-yl)acetamide (160.00 mg, 63.75%) as yellow solid. LC/MS: mass calcd. For C₂₄H₂₁ClN₆O₂S: 492.11, found: 493.20 [M+H]⁺.

### Synthesis of (S)-2-(4-(4-chlorophenyl)-2,3,9-trimethyl-6H-thieno[3,2-f][1,2,4]triazolo[4,3-a][1,4]diazepin-6-yl)-N-(piperidin-4-yl)acetamide

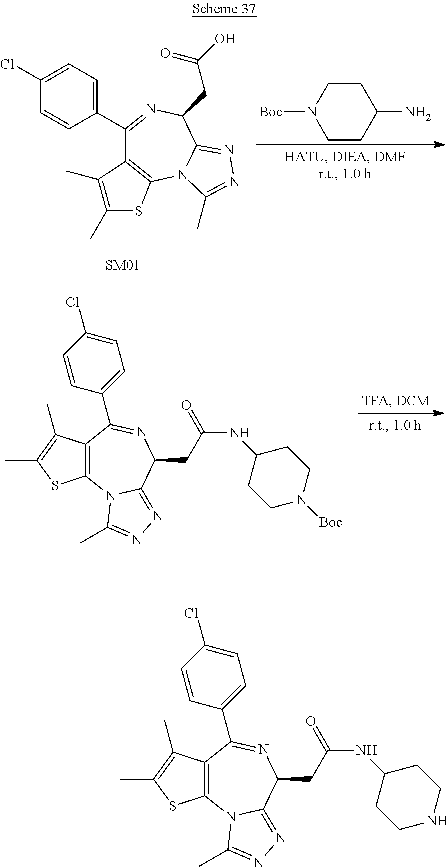 [SMILES: Cc1sc2c(c1C)C(c1ccc(Cl)cc1)=N[C@@H](CC(=O)O)c1nnc(C)n1-2 ; CC(C)(C)OC(=O)N1CCC(N)CC1 ; Cc1sc2c(c1C)C(c1ccc(Cl)cc1)=N[C@@H](CC(=O)NC1CCN(C(=O)OC(C)(C)C)CC1)c1nnc(C)n1-2 ; Cc1sc2c(c1C)C(c1ccc(Cl)cc1)=N[C@@H](CC(=O)NC1CCNCC1)c1nnc(C)n1-2]

### Step 1: Synthesis of tert-butyl (S)-4-(2-(4-(4-chlorophenyl)-2,3,9-trimethyl-6H-thieno[3,2-f][1,2,4]triazolo[4,3-a][1,4]diazepin-6-yl)acetamido)piperidine-1-carboxylate

The procedure was the same as tert-butyl (S)-4-(4-(2-(4-(4-chlorophenyl)-2,3,9-trimethyl-6H-thieno[3,2-f][1,2,4]triazolo[4,3-a][1,4]diazepin-6-yl)acetamido)phenyl)piperazine-1-carboxylate. 1.00 g of (S)-2-(4-(4-chlorophenyl)-2,3,9-trimethyl-6H-thieno[3,2-f][1,2,4]triazolo[4,3-a][1,4]diazepin-6-yl)acetic acid was used, 1.30 g of tert-butyl (S)-4-(2-(4-(4-chlorophenyl)-2,3,9-trimethyl-6H-thieno[3,2-f][1,2,4]triazolo[4,3-a][1,4]diazepin-6-yl)acetamido)piperidine-1-carboxylate was obtained as yellow solid (80.43% yield). LCMS: mass calcd. For C₂₉H₃₅ClN₆O₃S: 582.22, found: 583.40 [M+H]⁺.

### Step 2: Synthesis of (S)-2-(4-(4-chlorophenyl)-2,3,9-trimethyl-6H-thieno[3,2-f][1,2,4]triazolo[4,3-a][1,4]diazepin-6-yl)-N-(piperidin-4-yl)acetamide

The procedure was the same as (S)-2-(4-(4-chlorophenyl)-2,3,9-trimethyl-6H-thieno[3,2-f][1,2,4]triazolo[4,3-a][1,4]diazepin-6-yl)-N-(4-(piperazin-1-yl)phenyl)acetamide. 700.00 mg of tert-butyl (S)-4-(2-(4-(4-chlorophenyl)-2,3,9-trimethyl-6H-thieno[3,2-f][1,2,4]triazolo[4,3-a][1,4]diazepin-6-yl)acetamido)piperidine-1-carboxylate was used, 700.00 mg of (S)-2-(4-(4-chlorophenyl)-2,3,9-trimethyl-6H-thieno[3,2-f][1,2,4]triazolo[4,3-a][1,4]diazepin-6-yl)-N-(piperidin-4-yl)acetamide was obtained as yellow oil. LCMS: mass calcd. For C₂₄H₂₇ClN₆OS: 482.17, found: 483.25 [M+H]⁺.

### Synthesis of (S)-2-(6-(4-chlorophenyl)-8-methoxy-1-methyl-4H-benzo[f][1,2,4]triazolo[4,3-a][1,4]diazepin-4-yl)-N-(piperidin-4-yl)acetamide

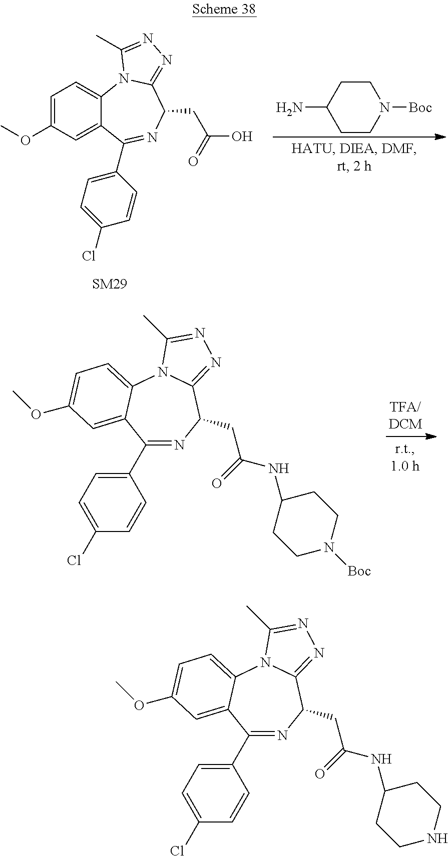 [SMILES: COc1ccc2c(c1)C(c1ccc(Cl)cc1)=N[C@@H](CC(=O)O)c1nnc(C)n1-2 ; CC(C)(C)OC(=O)N1CCC(N)CC1 ; COc1ccc2c(c1)C(c1ccc(Cl)cc1)=N[C@@H](CC(=O)NC1CCN(C(=O)OC(C)(C)C)CC1)c1nnc(C)n1-2 ; COc1ccc2c(c1)C(c1ccc(Cl)cc1)=N[C@@H](CC(=O)NC1CCNCC1)c1nnc(C)n1-2]

### Step 1: Synthesis of tert-butyl (S)-4-(2-(6-(4-chlorophenyl)-8-methoxy-1-methyl-4H-benzo[f][1,2,4]triazolo[4,3-a][1,4]diazepin-4-yl)acetamido)piperidine-1-carboxylate

The procedure was the same as tert-butyl (S)-4-(4-(2-(4-(4-chlorophenyl)-2,3,9-trimethyl-6H-thieno[3,2-f][1,2,4]triazolo[4,3-a][1,4]diazepin-6-yl)acetamido)phenyl)piperazine-1-carboxylate. 800.00 mg of (S)-2-(6-(4-chlorophenyl)-8-methoxy-1-methyl-4H-benzo[f][1,2,4]triazolo[4,3-a][1,4]diazepin-4-yl)acetic acid was used, 1.00 g of tert-butyl (S)-4-(2-(6-(4-chlorophenyl)-8-methoxy-1-methyl-4H-benzo[f][1,2,4]triazolo[4,3-a][1,4]diazepin-4-yl)acetamido)piperidine-1-carboxylate was obtained as white solid (79.74% yield). LCMS: mass calcd. For C₃₀H₃₅ClN₆O₄: 578.24, found: 579.15 [M+H]⁺.

### Step 2: Synthesis of (S)-2-(6-(4-chlorophenyl)-8-methoxy-1-methyl-4H-benzo[f][1,2,4]triazolo[4,3-a][1,4]diazepin-4-yl)-N-(piperidin-4-yl)acetamide

The procedure was the same as (S)-2-(4-(4-chlorophenyl)-2,3,9-trimethyl-6H-thieno[3,2-f][1,2,4]triazolo[4,3-a][1,4]diazepin-6-yl)-N-(4-(piperazin-1-yl)phenyl)acetamide. 800.00 mg of tert-butyl (S)-4-(2-(6-(4-chlorophenyl)-8-methoxy-1-methyl-4H-benzo[f][1,2,4]triazolo[4,3-a][1,4]diazepin-4-yl)acetamido)piperidine-1-carboxylate was used, 800.00 mg crude of (S)-2-(6-(4-chlorophenyl)-8-methoxy-1-methyl-4H-benzo[f][1,2,4]triazolo[4,3-a][1,4]diazepin-4-yl)-N-(piperidin-4-yl)acetamide was obtained as yellow oil. LCMS: mass calcd. For C₂₅H₂₇ClN₆O₂: 478.19, found: 479.20 [M+H]⁺.

### Example 1D: Synthesis of Transcription Modulator Molecule Compounds of the Disclosure

Many synthetic strategies have been applied to assemble DNA-binding moieties, protein-binding moieties and the linkers to form the final transcription modulator molecules. The representative examples of strategies (types of reactions) are illustrated below and throughout the experimental.

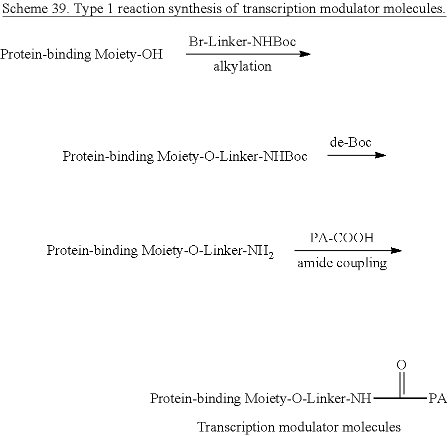

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 13)

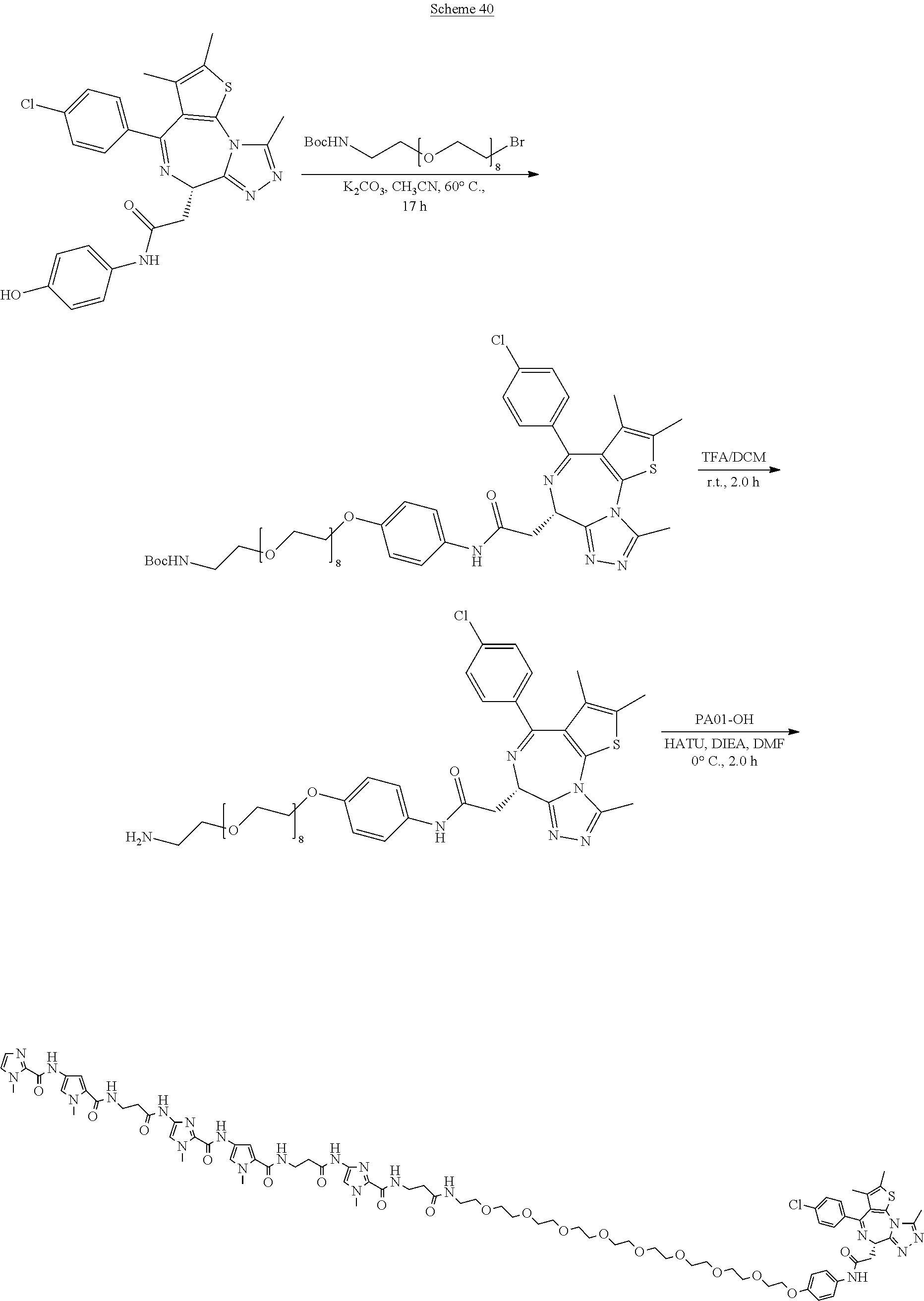

### Step 1: Synthesis of tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate

To a solution of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide (150.00 mg, 0.31 mmol, 1.00 equiv) in MeCN (4.00 mL) was added tert-butyl N-(26-bromo-3,6,9,12,15,18,21,24-octaoxahexacosan-1-yl) carbamate (175.77 mg, 0.31 mmol, 1.00 equiv) and K₂CO₃ (84.27 mg, 0.61 mmol, 2.00 equiv). Then the reaction mixture was stirred at 60 degrees C. for 17 h. The solid was filtrated out and the filtration was concentrated. The residue was purified by silica gel column chromatography, eluted with DCM:MeOH=10:1. Tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate (350.00 mg, quantitative yield) as white solid. LCMS: mass calcd. For C₄₈H₆₇ClN₆O₁₂S: 986.42, found: 1009.60 [M+Na]⁺.

### Step 2: Synthesis of (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide

To a solution of Tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate (350.00 mg, 0.35 mmol, 1.00 equiv) in DCM (4.00 mL) and TFA (1.00 mL) was stirred at room temperature for 2 h. The mixture was concentrated to afford 350.00 mg crude of (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide as yellow oil. LCMS: mass calcd. For C₄₃H₅₉ClN₆O₁₀S: 886.37, found: 887.50[M+H]⁺.

### Step 3: Synthesis of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

To a solution of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (180.00 mg, 0.22 mmol, 1.10 equiv) in DMF (3.00 mL) was added HATU (123.71 mg, 0.33 mmol, 1.50 equiv), (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide (157.00 mg, 0.15 mmol, 1.00 equiv) and DIPEA (84.10 mg, 0.65 mmol, 3.00 equiv). Then the reaction was stirred at 0 degrees C. for 2 h. The reaction mixture was filtered and the filtration in DMF (3 mL) was purified by Prep-HPLC with the following conditions: Column: XBridge Shield RP18 OBD Column, 19*250 mm, 10 um; Mobile Phase A: Water (10 MMOL/L NH4HCO3), Mobile Phase B: ACN; Flow rate: 25 mL/min; Gradient: 40 B to 42 B in 13 min; 254 nm; RT1: 11.65. The fractions were combined and lyophilized directly to afford (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (27.70 mg, 8.93%) as white solid. HRMS: mass calcd. For C₇₉H₁₀₀ClN₂₁O₁₈S: 1697.6964, found: 1698.7034 [M+H]⁺.

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 21)

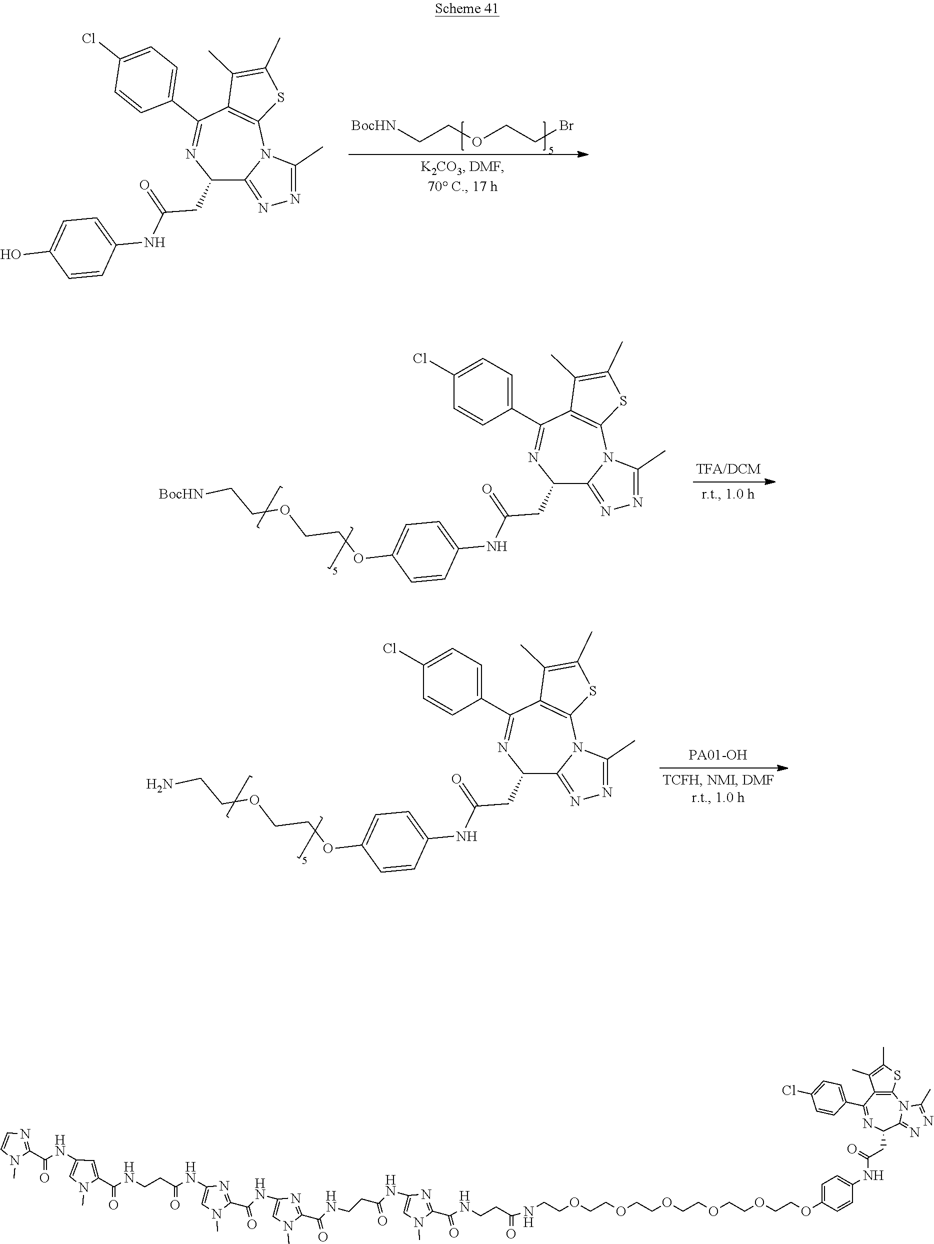

### Step 1: Synthesis of tert-butyl (S)-(17-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaheptadecyl)carbamate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 70.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 50.00 mg of tert-butyl (S)-(17-(4-(2-(4-(4-chlorophenyl)-2,3,9-trimethyl-6H-thieno[3,2-f][1,2,4]triazolo [4,3-a][1,4]diazepin-6-yl)acetamido)phenoxy)-3,6,9,12,15-pentaoxaheptadecyl)carbamate was obtained as yellow oil (34.57% yield). LC/MS: mass calcd. For C₄₂H₅₅ClN₆O₉S: 854.34, found: 856.60 [M+H]⁺.

### Step 2: Synthesis of (S)—N-(4-((17-amino-3,6,9,12,15-pentaoxaheptadecyl)oxy) phenyl)-2-(4-(4-chlorophenyl)-2,3,9-trimethyl-6H-thieno[3,2-f][1,2,4]triazolo[4,3-a][1,4]diazepin-6-yl)acetamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 50.00 mg of tert-butyl (S)-(17-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaheptadecyl)carbamate was used and 50.00 mg crude of (S)—N-(4-((17-amino-3,6,9,12,15-pentaoxaheptadecyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide was obtained as yellow oil. LC/MS: mass calcd. For C₃₇H₄₇ClN₆O₇S: 754.29, found: 755.45 [M+H]⁺.

### Step 3: Synthesis of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

Into a 8 ml flask was added 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanonic acid (55.00 mg, 0.07 mmol, 1.00 equiv), DMF (2.00 mL), NMI (32.65 mg, 0.40 mmol, 6.00 equiv), TCFH (22.32 mg, 0. mmol, 1.20 equiv), the mixture was stirred at room temperature for 5 mins, then (S)—N-(4-((17-amino-3,6,9,12,15-pentaoxaheptadecyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide (50.00 mg, 0.07 mmol, 1.00 equiv) was added. The reaction was stirred at room temperature for 1 h. The reaction mixture was filtered and the filtration in DMF (3 mL) was purified by Perp-HPLC with the following conditions: Column: XBridge Shield RP18 OBD Column, 19*250 mm, 10 um; Mobile Phase A: Water (10 MMOL/L NH₄HCO₃+0.1% NH₃.H₂O), Mobile Phase B: ACN; Flow rate: 25 mL/min; Gradient: 40 B to 43 B in 12 min; 254 nm; RT1: 10.85. The fractions were combined and lyophilized directly. This resulted in (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (10.00 mg, 9.25%) as white solid. HRMS: mass calcd. For C₇₃H₈₈ClN₂₁O₅S: 1565.6178, found: 1566.6221 [M+H]⁺.

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-22-oxo-3,6,9,12,15,18-hexaoxa-21-azatetracosan-24-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 22)

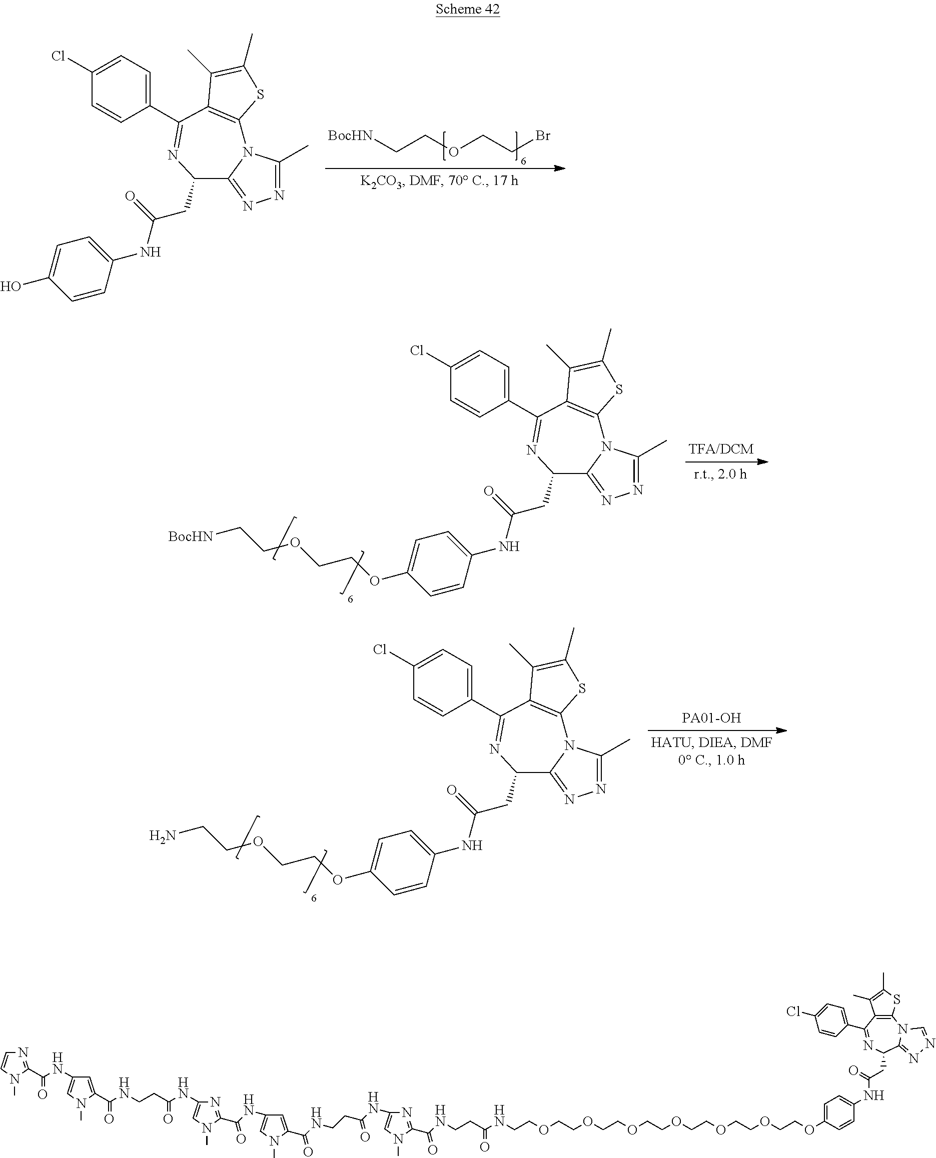

### Step 1: Synthesis of tert-butyl (S)-(20-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18-hexaoxaicosyl)carbamate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 100.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 120.00 mg of tert-butyl (S)-(20-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18-hexaoxaicosyl)carbamate was obtained as yellow oil (65.64% yield). LCMS: mass calcd. For C₄₄H₅₉ClN₆O₁₀S: 898.37, found: 921.55 [M+Na]+.

### Step 2: Synthesis of (S)—N-(4-((20-amino-3,6,9,12,15,18-hexaoxaicosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 120.00 mg of tert-butyl(S)-(20-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18-hexaoxaicosyl)carbamate was used, 105.00 mg crude of (S)—N-(4-((20-amino-3,6,9,12,15,18-hexaoxaicosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide was obtained as yellow oil. LCMS: mass calcd. For C₃₉H₅₁ClN₆O₈S: 798.32, found: 799.45 [M+H]⁺.

### Step 3: Synthesis of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-22-oxo-3,6,9,12,15,18-hexaoxa-21-azatetracosan-24-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 101.15 mg of (S)—N-(4-((20-amino-3,6,9,12,15,18-hexaoxaicosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide was used, 29.10 mg of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-22-oxo-3,6,9,12,15,18-hexaoxa-21-azatetracosan-24-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (13.86% yield). HRMS: mass calcd. For C₇₅H₉₂ClN₂₁O₁₆S: 1609.6440, found: 1610.6553 [M+H]⁺.

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-25-oxo-3,6,9,12,15,18,21-heptaoxa-24-azaheptacosan-27-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 23)

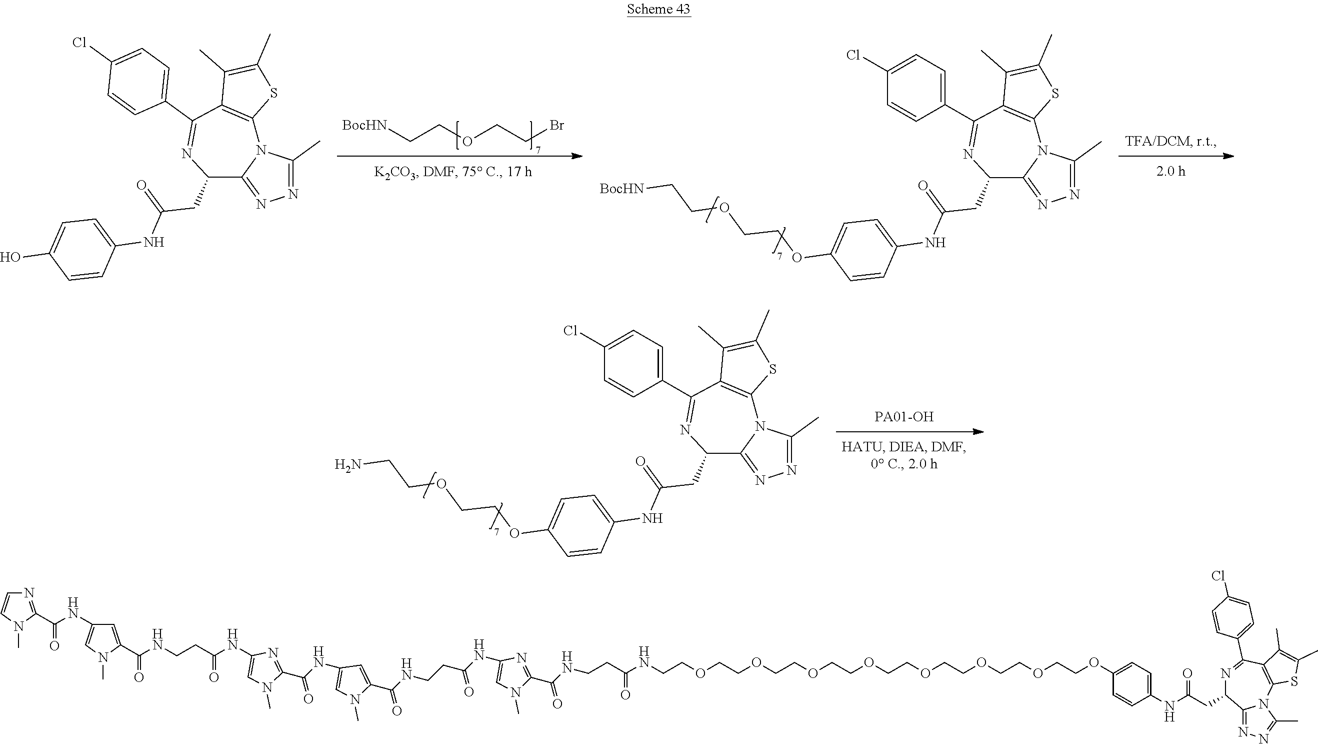

### Step 1: Synthesis of tert-butyl (S)-(23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosyl)carbamate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 100.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 80.00 mg of tert-butyl (S)-(23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosyl)carbamate was obtained as yellow oil (41.71% yield). LCMS: mass calcd. For C₄₆H₆₃ClN₆O₁₁S: 942.40, found: 943.60 [M+H]⁺.

### Step 2: Synthesis of (S)—N-(4-((23-amino-3,6,9,12,15,18,21-heptaoxatricosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 80.00 mg of tert-butyl (S)-(23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosyl)carbamate was used, 80.00 mg crude of (S)—N-(4-((23-amino-3,6,9,12,15,18,21-heptaoxatricosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide was obtained as yellow oil. LCMS: mass calcd. For C₄H₅₅ClN₆O₉S: 842.34, found: 843.30 [M+H]⁺.

### Step 3: Synthesis of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-25-oxo-3,6,9,12,15,18,21-heptaoxa-24-azaheptacosan-27-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 80.00 mg crude of (S)—N-(4-((23-amino-3,6,9,12,15,18,21-heptaoxatricosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide was used, 32.00 mg of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-25-oxo-3,6,9,12,15,18,21-heptaoxa-24-azaheptacosan-27-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (21.80% yield). HRMS: mass calcd. For C₇₇H₉₆ClN₂₁O₁₇S: 1653.6702, found: 1654.6807 [M+H]⁺.

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-31-oxo-3,6,9,12,15,18,21,24,27-nonaoxa-30-azatritriacontan-33-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 24)

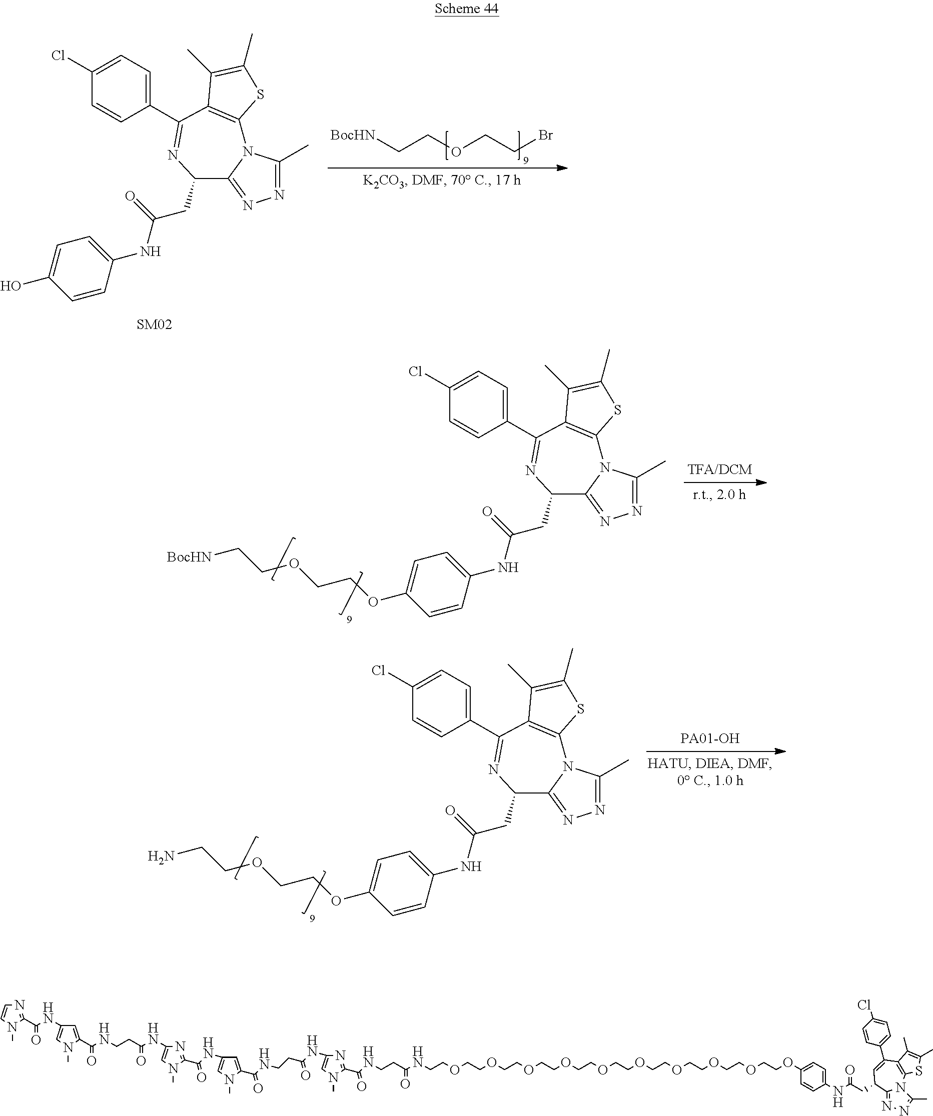

### Step 1: Synthesis of tert-butyl (S)-(29-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24,27-nonaoxanonacosyl)carbamate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 100.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 150.00 mg of tert-butyl (S)-(29-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24,27-nonaoxanonacosyl)carbamate was obtained as light yellow oil (71.53% yield). LCMS: mass calcd. For C₅₀H₇₁ClN₆O₁₃S: 1030.45, found: 1031.70 [M+H]⁺.

### Step 2: Synthesis of (S)—N-(4-((29-amino-3,6,9,12,15,18,21,24,27-nonaoxanonacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 150.00 mg of tert-butyl (S)-(29-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24,27-nonaoxanonacosyl)carbamate was used, 150.00 mg crude of (S)—N-(4-((29-amino-3,6,9,12,15,18,21,24,27-nonaoxanonacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide was obtained as yellow oil. LCMS: mass calcd. For C₄₅H₆₃ClN₆O₁₁S: 930.40, found: 931.55 [M+H]⁺.

### Step 3: Synthesis of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-31-oxo-3,6,9,12,15,18,21,24,27-nonaoxa-30-azatritriacontan-33-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 100.00 mg crude of (S)—N-(4-((29-amino-3,6,9,12,15,18,21,24,27-nonaoxanonacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide was used, 34.60 mg of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido) phenoxy)-31-oxo-3,6,9,12,15,18,21,24,27-nonaoxa-30-azatritriacontan-33-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (16.47% yield). HRMS: mass calcd. For C₈₁H₁₀ClN₂₁O₁₉S: 1741.7227, found: 1742.7294 [M+H]⁺.

### (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-25-oxo-3,6,9,12,15,18,21-heptaoxa-24-azaheptacosan-27-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (Compound 32)

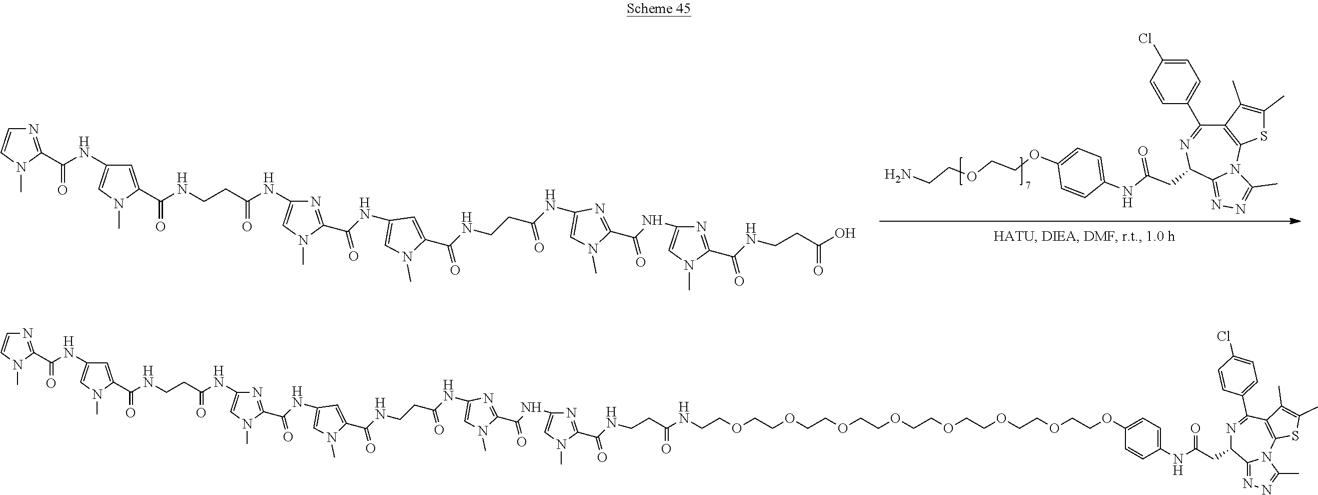

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 140.00 mg crude of (S)—N-(4-((23-amino-3,6,9,12,15,18,21-heptaoxatricosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide was used, 47.90 mg of (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-25-oxo-3,6,9,12,15,18,21-heptaoxa-24-azaheptacosan-27-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide was obtained as white solid (15.17% yield). HRMS: mass calcd. For C₈₃H₁₀₂ClN₂₃O₁₈S: 1775.7182, found: 1776.7324 [M+H]⁺.

### (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (Compound 33)

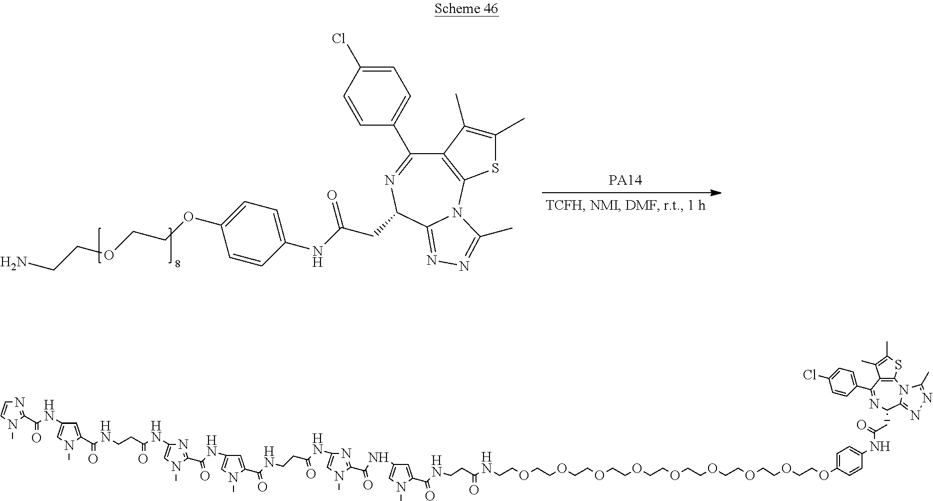

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 93.23 mg of (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide was used, 47.70 mg of (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide was obtained as white solid (23.70% yield). HRMS: mass calcd. For C₈₅H₁₀₆ClN₂₃O₁₉S: 1819.7445, found: 1820.7548 [M+H]⁺.

### (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-31-oxo-3,6,9,12,15,18,21,24,27-nonaoxa-30-azatritriacontan-33-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (Compound 34)

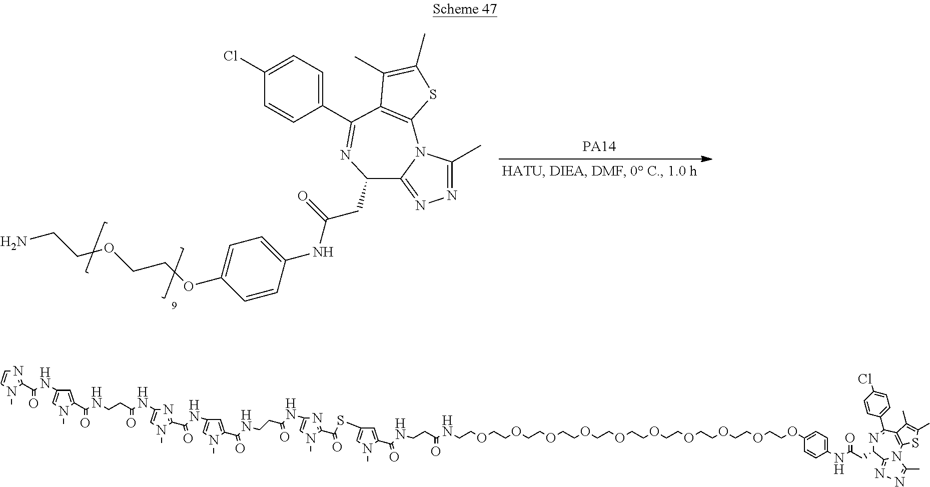

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 97.85 mg of (S)—N-(4-((29-amino-3,6,9,12,15,18,21,24,27-nonaoxanonacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide was used, 53.30 mg of (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-31-oxo-3,6,9,12,15,18,21,24,27-nonaoxa-30-azatritriacontan-33-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide was obtained as white solid (26.06% yield). HRMS: mass calcd. For C₈₇H₁₁₀ClN₂₃O₂₀S: 1863.7707, found: 1864.7745 [M+H]⁺.

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-4-palmitamido-1H-imidazole-2-carboxamido)-1H-pyrrole-2-carboxamido)propanamido)-1H-imidazole-2-carboxamide (Compound 67)

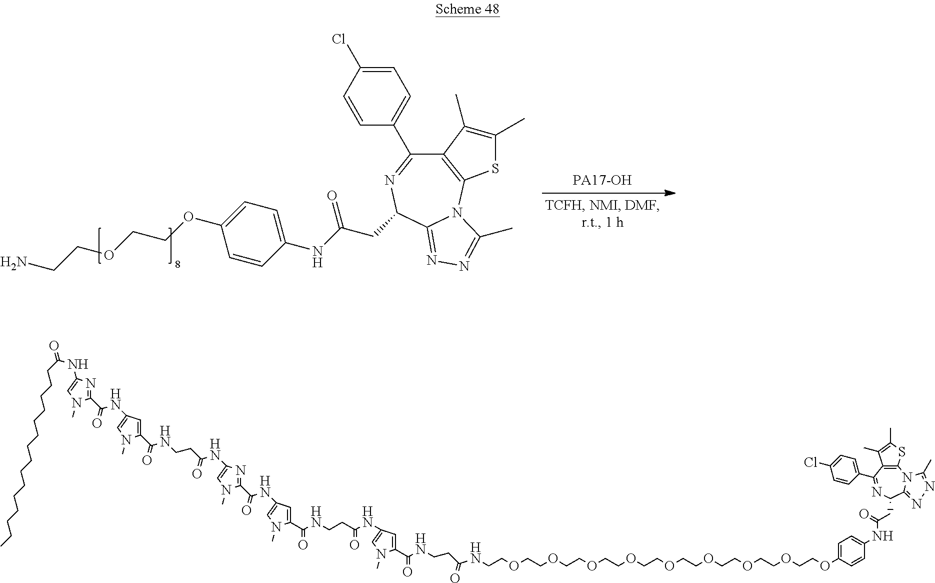

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 60.00 mg of (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide (1.33 equiv) was used, 10.33 mg of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-4-palmitamido-1H-imidazole-2-carboxamido)-1H-pyrrole-2-carboxamido)propanamido)-1H-imidazole-2-carboxamide was obtained as white solid (9.99% yield). HRMS: mass calcd. For C₉₅H₁₃₁ClN₂₂O₁₉S: 1950.9370, found: 1951.9484 [M+H]⁺.

### (S)—N-(5-((3-((2-((1-(4-(2-(6-(4-chlorophenyl)-8-methoxy-1-methyl-4H-benzo[f][1,2,4]triazolo[4,3-a][1,4]diazepin-4-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 25)

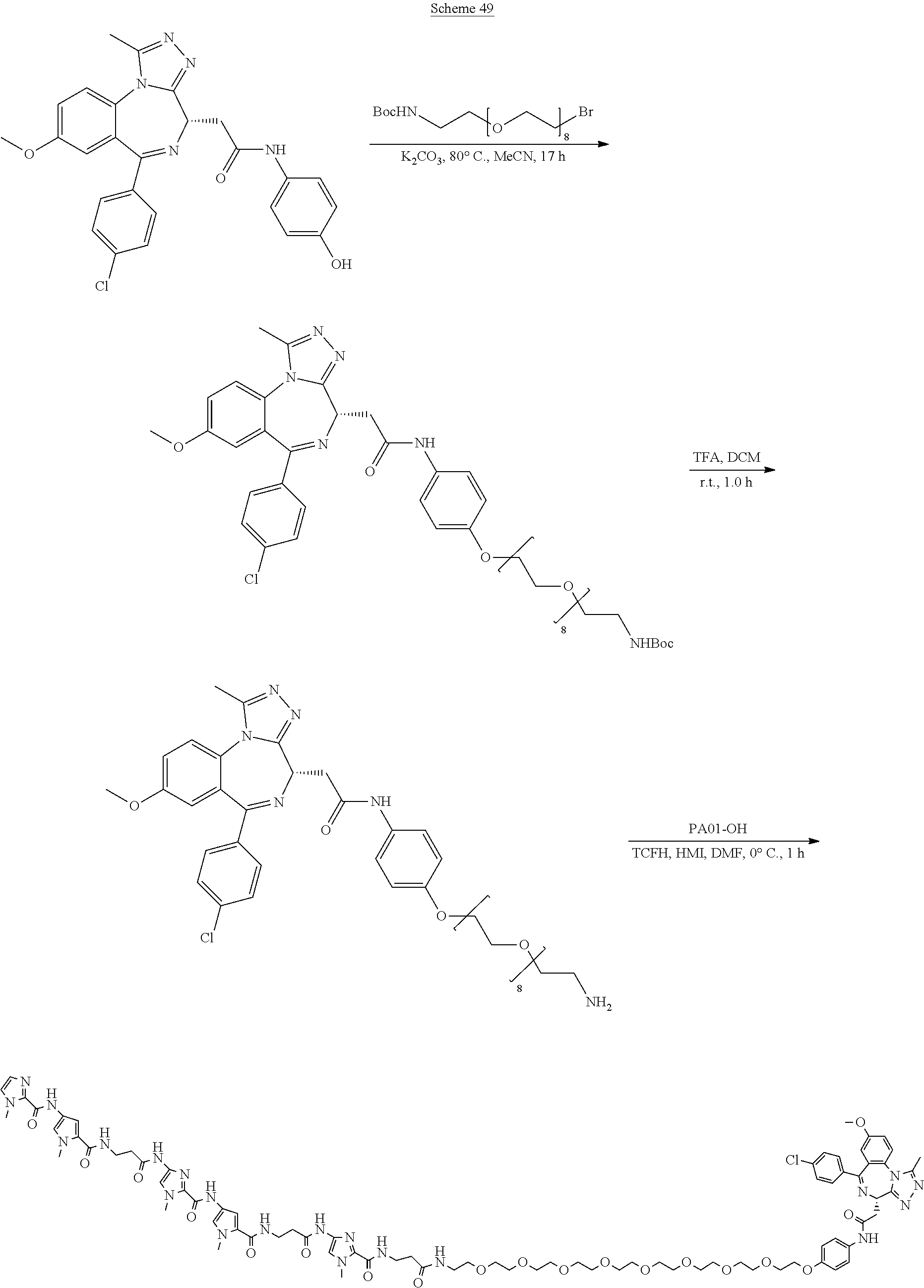

### Step 1: Synthesis of tert-butyl (S)-(26-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15,18,21,24-octaoxahexacosyl)carbamate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 130.00 mg of (S)-2-(6-(4-chlorophenyl)-8-methoxy-1-methyl-4H-benzo[f][1,2,4]triazolo[4,3-a][1,4]diazepin-4-yl)-N-(4-hydroxyphenyl)acetamide was used, 140.00 mg of tert-butyl (S)-(26-(4-(2-(6-(4-chlorophenyl)-8-methoxy-1-methyl-4H-benzo[f][1,2,4]triazolo [4,3-a][1,4]diazepin-4-yl)acetamido)phenoxy)-3,6,9,12,15,18,21,24-octaoxahexacosyl)carbamate was obtained as yellow oil (45.41% yield). LC/MS: mass calcd. For C₄₉H₆₇ClN₆O₁₃: 982.45, found: 983.65 [M+H]⁺.

### Step 2: Synthesis of (S)—N-(4-((26-amino-3,6,9,12,15,18,21,24-octaoxahexacosyl)oxy)phenyl)-2-(6-(4-chlorophenyl)-8-methoxy-1-methyl-4H-benzo[f][1,2,4]triazolo[4,3-a][1,4]diazepin-4-yl)acetamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 140.00 mg of tert-butyl (S)-(26-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15,18,21,24-octaoxahexacosyl)carbamate was used, 130.00 mg crude of (S)—N-(4-((26-amino-3,6,9,12,15,18,21,24-octaoxahexacosyl)oxy)phenyl)-2-(6-(4-chlorophenyl)-8-methoxy-1-methyl-4H-benzo[f][1,2,4]triazolo[4,3-a][1,4]diazepin-4-yl)acetamide was obtained as yellow oil. LC/MS: mass calcd. For C₄₄H₅₉ClN₆O₁₁: 882.39, found: 883.60 [M+H]⁺.

### Step 3: Synthesis of (S)—N-(5-((3-((2-((1-(4-(2-(6-(4-chlorophenyl)-8-methoxy-1-methyl-4H-benzo[f][1,2,4]triazolo[4,3-a][1,4]diazepin-4-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 130.00 mg crude of (S)—N-(4-((26-amino-3,6,9,12,15,18,21,24-octaoxahexacosyl)oxy)phenyl)-2-(6-(4-chlorophenyl)-8-methoxy-1-methyl-4H-benzo[f][1,2,4]triazolo[4,3-a][1,4]diazepin-4-yl)acetamide was used, 27.30 mg of (S)—N-(5-((3-((2-((1-(4-(2-(6-(4-chlorophenyl)-8-methoxy-1-methyl-4H-benzo[f][1,2,4]triazolo[4,3-a][1,4]diazepin-4-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (10.66% yield). HRMS: mass calcd. For C₈₀H₁₀₀ClN₂₁O₁₉: 1693.72, found: 1694.7252 [M+H]⁺.

### (R)—N-(5-((3-((2-((1-(4-(2-(6-(4-chlorophenyl)-8-methoxy-1-methyl-4H-benzo[f][1,2,4]triazolo[4,3-a][1,4]diazepin-4-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 26)

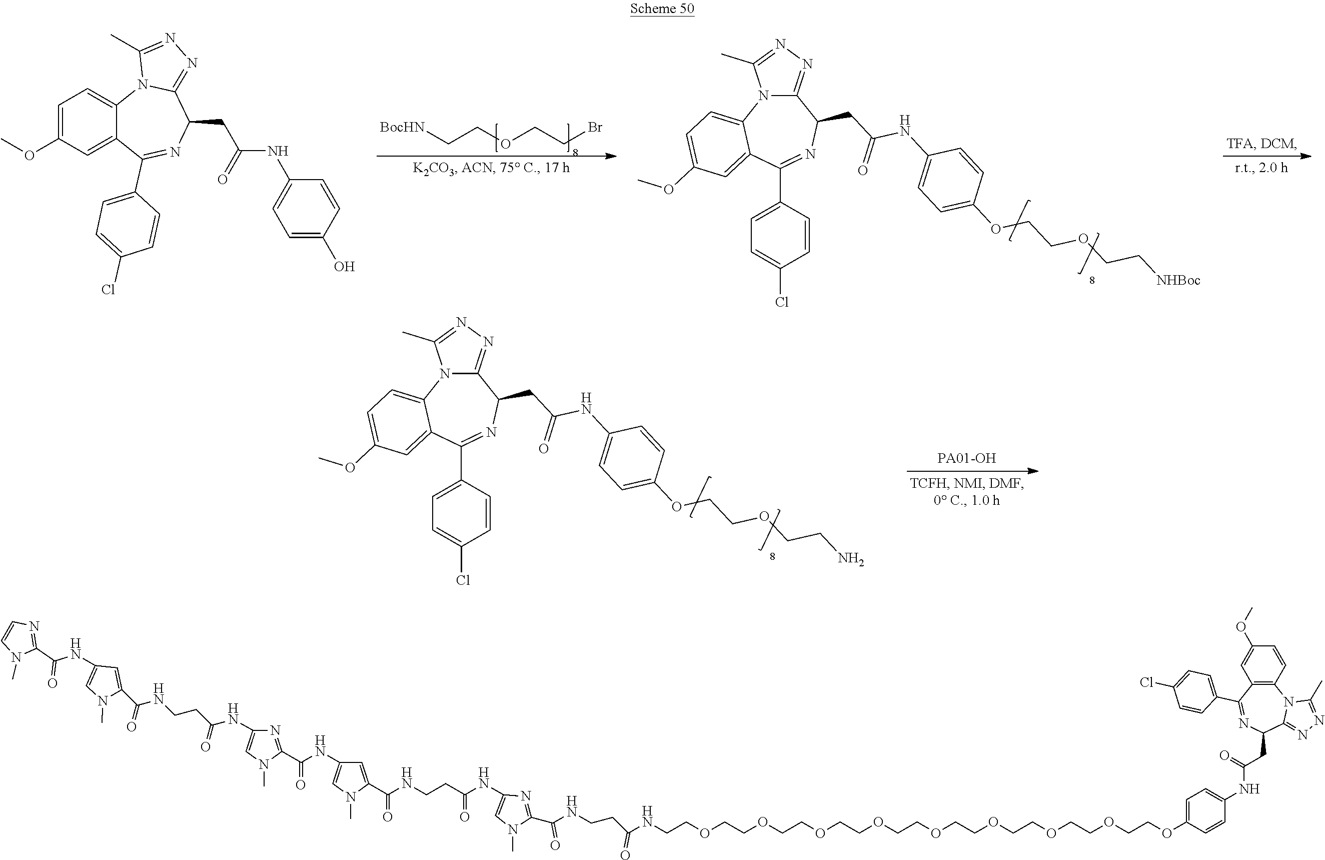

### Step 1: Synthesis of tert-butyl (R)-(26-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15,18,21,24-octaoxahexacosyl)carbamate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 120.00 mg of (R)-2-(6-(4-chlorophenyl)-8-methoxy-1-methyl-4H-benzo[f][1,2,4]triazolo[4,3-a][1,4]diazepin-4-yl)-N-(4-hydroxyphenyl)acetamide was used, 350.00 mg crude of tert-butyl (R)-(26-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15,18,21,24-octaoxahexacosyl)carbamate (quantitative yield) was obtained as yellow oil. LCMS: mass calcd. For C₄₉H₆₇ClN₆O₁₃: 982.45, found: 1005.60 [M+Na]⁺.

### Step 2: Synthesis of (R)—N-(4-((26-amino-3,6,9,12,15,18,21,24-octaoxahexacosyl)oxy)phenyl)-2-(6-(4-chlorophenyl)-8-methoxy-1-methyl-4H-benzo[f][1,2,4]triazolo[4,3-a][1,4]diazepin-4-yl)acetamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 350.00 mg crude of tert-butyl (R)-(26-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15,18,21,24-octaoxahexacosyl)carbamate was used, 350.00 mg crude of (R)—N-(4-((26-amino-3,6,9,12,15,18,21,24-octaoxahexacosyl)oxy)phenyl)-2-(6-(4-chlorophenyl)-8-methoxy-1-methyl-4H-benzo[f][1,2,4]triazolo[4,3-a][1,4]diazepin-4-yl)acetamide was obtained as yellow oil. LCMS: mass calcd. For C₄₄H₅₉ClN₆O₁₁: 882.39, found: 443.25 [M/2+H]⁺.

### Step 3: Synthesis of (R)—N-(5-((3-((2-((1-(4-(2-(6-(4-chlorophenyl)-8-methoxy-1-methyl-4H-benzo[f][1,2,4]triazolo[4,3-a][1,4]diazepin-4-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 350.00 mg crude of (R)—N-(4-((26-amino-3,6,9,12,15,18,21,24-octaoxahexacosyl)oxy)phenyl)-2-(6-(4-chlorophenyl)-8-methoxy-1-methyl-4H-benzo[f][1,2,4]triazolo[4,3-a][1,4]diazepin-4-yl)acetamide was used, 38.3 mg of (R)—N-(5-((3-((2-((1-(4-(2-(6-(4-chlorophenyl)-8-methoxy-1-methyl-4H-benzo[f][1,2,4]triazolo[4,3-a][1,4]diazepin-4-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (6.60% yield). HRMS: mass calcd. For C₈₀H₁₀₀ClN₂₁O₁₉: 1693.7193, found: 1694.7240 [M+H]⁺.

### (S)—N-(5-((3-((2-((1-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 65)

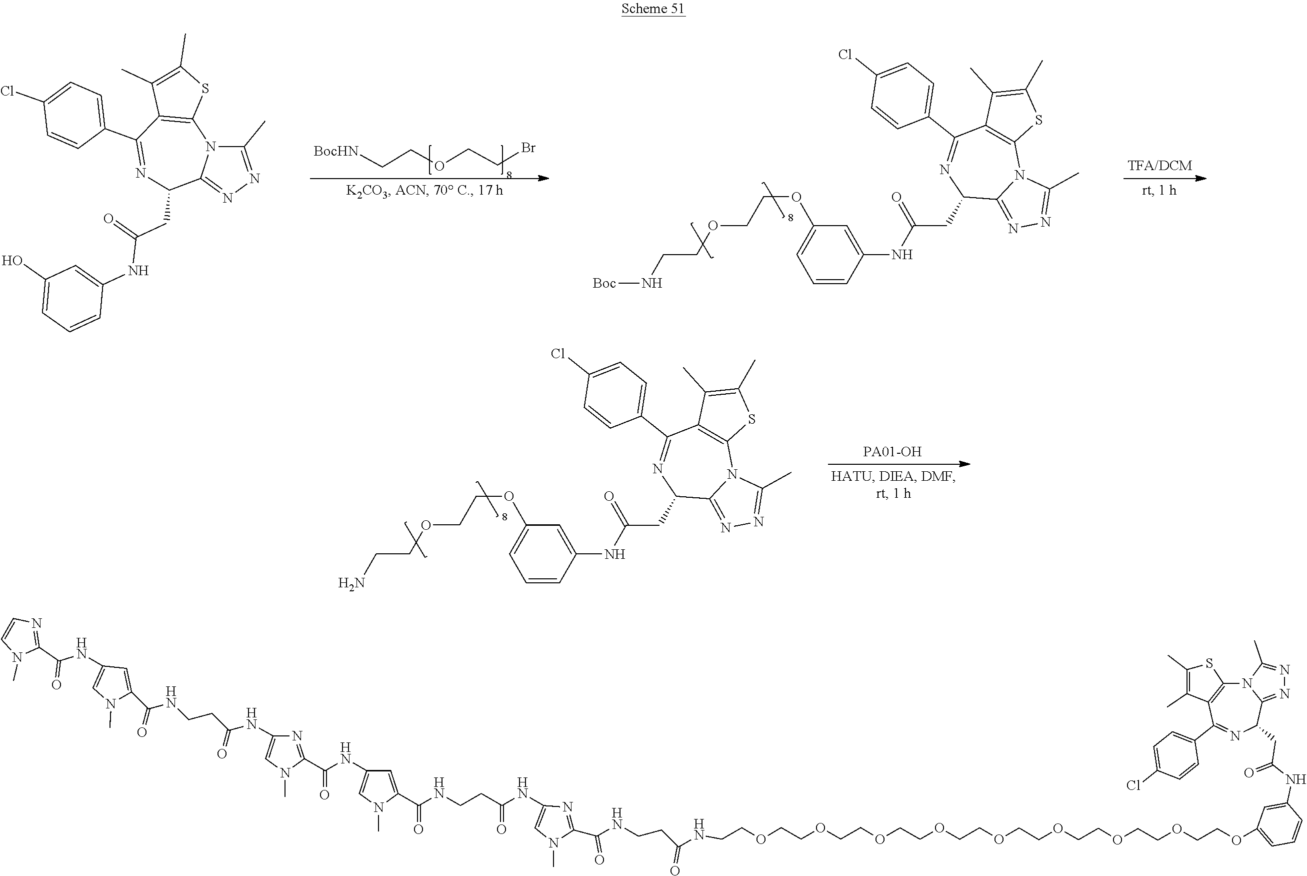

### Step 1: Synthesis of tert-butyl (S)-(26-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 100.00 mg of (S)-2-(4-(4-chlorophenyl)-2,3,9-trimethyl-6H-thieno[3,2-f][1,2,4]triazolo[4,3-a][1,4]diazepin-6-yl)-N-(3-hydroxyphenyl)acetamide was used, 150.00 mg of tert-butyl (S)-(26-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate was obtained as colorless oil (67.70% yield). LC/MS: mass calcd. For C₄₈H₆₇ClN₆O₁₂S: 986.42, found: 494.50 [M/2+H]⁺.

### Step 2: Synthesis of (S)—N-(3-((26-amino-3,6,9,12,15,18,21,24-octaoxahexacosyl) oxy)phenyl)-2-(4-(4-chlorophenyl)-2,3,9-trimethyl-6H-thieno[3,2-f][1,2,4]triazolo[4,3-a][1,4]diazepin-6-yl)acetamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 140.00 mg of tert-butyl (S)-(26-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate was used, 140.00 mg crude of (S)—N-(3-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide was obtained as yellow oil. LCMS: mass calcd. For C₄₃H₅₉ClN₆O₁₀S: 886.37, found: 887.65 [M+H]⁺.

### Step 3: 3-((2-((1-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 140.00 mg of (S)—N-(3-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide was used, 21.20 mg of 3-((2-((1-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (8.32% yield). HRMS: mass calcd. For C₇₉H₁₀₀ClN₂₁O₁₈S: 1697.6964, found: 1698.7102 [M+H]⁺.

### (S)—N-(5-((3-((2-((1-((5-(2-(4-(4-chlorophenyl)-2,3,9-trimethyl-6H-thieno[3,2-f][1,2,4]triazolo[4,3-a][1,4]diazepin-6-yl)acetamido)pyridin-3-yl)oxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 103)

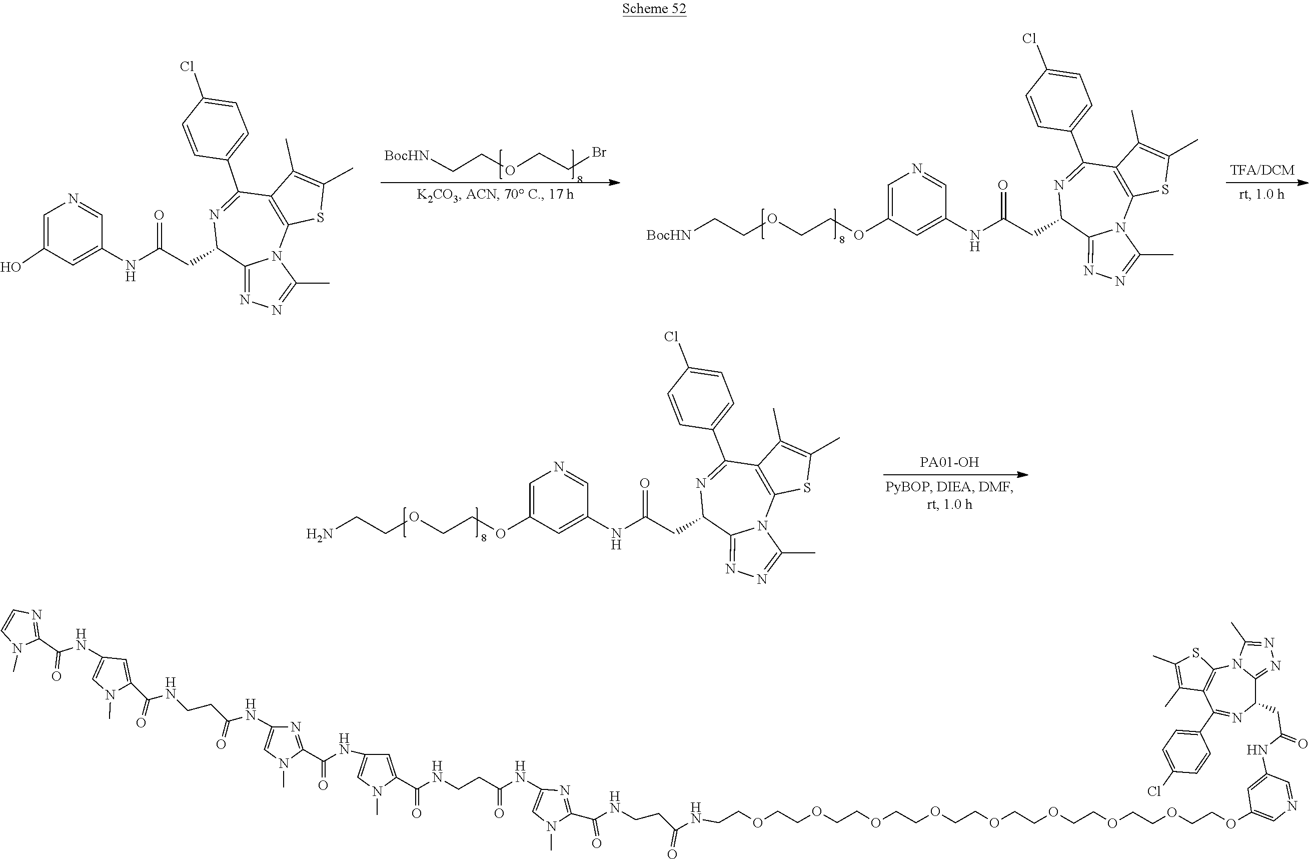

### Step 1: Synthesis of tert-butyl (S)-(26-((5-(2-(4-(4-chlorophenyl)-2,3,9-trimethyl-6H-thieno[3,2-f][1,2,4]triazolo[4,3-a][1,4]diazepin-6-yl)acetamido)pyridin-3-yl)oxy)-3,6,9,12,15,18,21,24-octaoxahexacosyl)carbamate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 135.00 mg of (S)-2-(4-(4-chlorophenyl)-2,3,9-trimethyl-6H-thieno[3,2-f][1,2,4]triazolo[4,3-a][1,4]diazepin-6-yl)-N-(5-hydroxypyridin-3-yl)acetamide was used, 156.00 mg of tert-butyl (S)-(26-((5-(2-(4-(4-chlorophenyl)-2,3,9-trimethyl-6H-thieno[3,2-f][1,2,4]triazolo[4,3-a][1,4]diazepin-6-yl)acetamido)pyridin-3-yl)oxy)-3,6,9,12,15,18,21,24-octaoxahexacosyl)carbamate was obtained as yellow solid (46.68% yield). LC/MS: mass calcd. For C₄₇H₆₆ClN₇O₁₂S: 987.42, found: 1010.90 [M+Na]⁺.

### Step 2: Synthesis of (S)—N-(5-((26-amino-3,6,9,12,15,18,21,24-octaoxahexacosyl)oxy)pyridin-3-yl)-2-(4-(4-chlorophenyl)-2,3,9-trimethyl-6H-thieno[3,2-f][1,2,4]triazolo[4,3-a][1,4]diazepin-6-yl)acetamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 90.00 mg of tert-butyl (S)-(26-((5-(2-(4-(4-chlorophenyl)-2,3,9-trimethyl-6H-thieno[3,2-f][1,2,4]triazolo[4,3-a][1,4]diazepin-6-yl)acetamido)pyridin-3-yl)oxy)-3,6,9,12,15,18,21,24-octaoxahexacosyl)carbamate was used, 90.00 mg crude of (S)—N-(5-((26-amino-3,6,9,12,15,18,21,24-octaoxahexacosyl)oxy)pyridin-3-yl)-2-(4-(4-chlorophenyl)-2,3,9-trimethyl-6H-thieno[3,2-f][1,2,4]triazolo[4,3-a][1,4]diazepin-6-yl)acetamide was obtained as yellow oil. LC/MS: mass calcd. For C₄₂H₅₈ClN₇O₁₀S: 887.37, found: 888.35 [M+H]⁺

### Step 3: Synthesis of (S)—N-(5-((3-((2-((1-((5-(2-(4-(4-chlorophenyl)-2,3,9-trimethyl-6H-thieno[3,2-f][1,2,4]triazolo[4,3-a][1,4]diazepin-6-yl)acetamido)pyridin-3-yl)oxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

To a stirred mixture of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (84.06 mg, 0.10 mmol, 1.00 equiv) and PyBOP (79.07 mg, 0.15 mmol, 1.50 equiv) in DMF (2.00 mL) was added (S)—N-(5-((26-amino-3,6,9,12,15,18,21,24-octaoxahexacosyl)oxy)pyridin-3-yl)-2-(4-(4-chlorophenyl)-2,3,9-trimethyl-6H-thieno[3,2-f][1,2,4]triazolo[4,3-a][1,4]diazepin-6-yl)acetamide (90.00 mg, 0.10 mmol, 1.00 equiv) and DIEA (130.92 mg, 1.01 mmol, 10.00 equiv) dropwise at 0 degrees C. The resulting mixture was stirred for 1 h at room temperature. After reaction, the reaction mixture was filtered and the filtrate in DMF (3 mL) was purified by Prep-HPLC with the following conditions: Column: YMC-Actus Triart C18, 20*250 mm, 5 m, 12 nm; Mobile Phase A: Water (10 MMOL/L NH₄HCO₃+0.1% NH₃.H₂O), Mobile Phase B: ACN; Flow rate: 25 mL/min; Gradient: 35% B to 43% B in 17 min, 43% B; Wave Length: 254 nm; RT1(min): 15.53. The fractions were combined and lyophilized directly to afford (S)—N-(5-((3-((2-((1-((5-(2-(4-(4-chlorophenyl)-2,3,9-trimethyl-6H-thieno[3,2-f][1,2,4]triazolo[4,3-a][1,4]diazepin-6-yl)acetamido)pyridin-3-yl)oxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (33.9 mg, 19.68%) as white solid. HRMS: mass calcd. For C₇₈H₉₉ClN₂₂O₁₈S: 1698.6917, found: 1699.7018 [M+H]⁺.

Type 2 Synthesis of Transcription Modulator Molecules

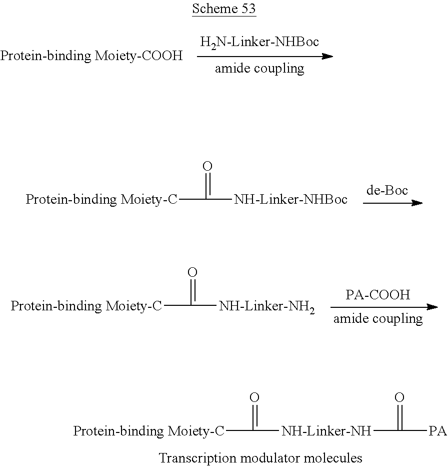

### N-(5-((3-((2-((1-(4-((2S,4R)-1-acetyl-4-((4-chlorophenyl)amino)-2-methyl-1,2,3,4-tetrahydroquinolin-6-yl)phenyl)-1,27-dioxo-5,8,11,14,17,20,23-heptaoxa-2,26-diazanonacosan-29-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 35)

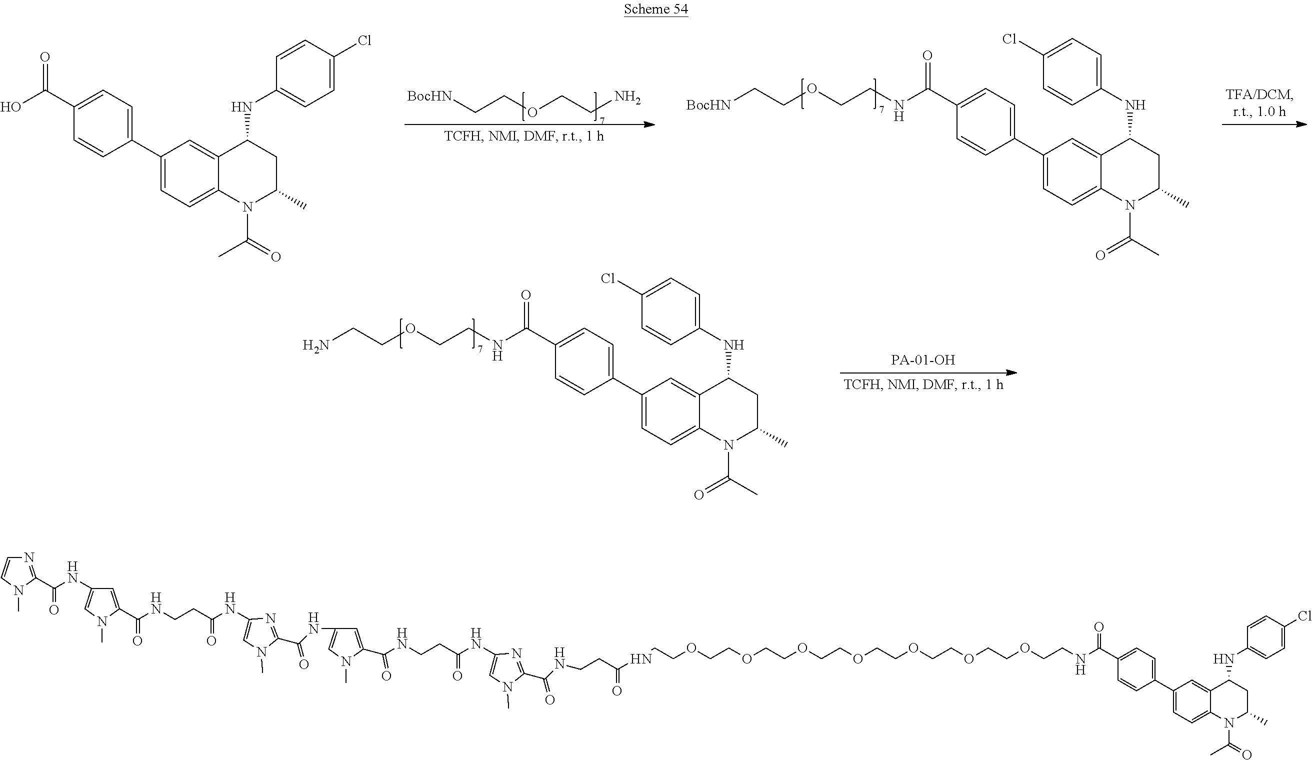

### Step 1: Synthesis of tert-butyl N-[23-([4-[(2S,4R)-1-acetyl-4-[(4-chlorophenyl)amino]-2-methyl-3,4-dihydro-2H-quinolin-6-yl]phenyl]formamido)-3,6,9,12,15,18,21-heptaoxatricosan-1-yl]carbamate

Into a 50 mL flask was added 4-[(2S,4R)-1-acetyl-4-[(4-chlorophenyl)amino]-2-methyl-3,4-dihydro-2H-quinolin-6-yl]benzoic acid (100.00 mg, 0.23 mmol, 1.00 equiv), DMF (5.00 mL), NMI (113.27 mg, 1.38 mmol, 6.00 equiv), TCFH (77.42 mg, 0.28 mmol, 1.20 equiv), the mixture was stirred at room temperature for 5 mins, then tert-butyl N-(23-amino-3,6,9,12,15,18,21-heptaoxatricosan-1-yl)carbamate (107.74 mg, 0.23 mmol, 1.00 equiv) was added, the reaction was stirred at room temperature for 1 h. The residue was purified by reverse flash chromatography with the following conditions: column, C18 silica gel; mobile phase: CH₃CN in water (0.05% TFA), 40% to 50% gradient in 20 min; detector, UV 254 nm. The fractions were combined and concentrated. This resulted in tert-butyl N-[23-([4-[(2S,4R)-1-acetyl-4-[(4-chlorophenyl)amino]-2-methyl-3,4-dihydro-2H-quinolin-6-yl]phenyl]formamido)-3,6,9,12,15,18,21-heptaoxatricosan-1-yl]carbamate (230.00 mg, quantitative yield) as a yellow oil. LC/MS: mass calcd. For C₄₆H₆₅ClN₄O₁₁: 884.43, found: 885.65 [M+H]⁺.

### Step 2: Synthesis of 4-[(2S,4R)-1-acetyl-4-[(4-chlorophenyl)amino]-2-methyl-3,4-dihydro-2H-quinolin-6-yl]-N-(23-amino-3,6,9,12,15,18,21-heptaoxatricosan-1-yl)benzamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 110.00 mg of tert-butyl N-[23-([4-[(2S,4R)-1-acetyl-4-[(4-chlorophenyl)amino]-2-methyl-3,4-dihydro-2H-quinolin-6-yl]phenyl]formamido)-3,6,9,12,15,18,21-heptaoxatricosan-1-yl]carbamate was used, 110.00 mg crude of 4-[(2S,4R)-1-acetyl-4-[(4-chlorophenyl)amino]-2-methyl-3,4-dihydro-2H-quinolin-6-yl]-N-(23-amino-3,6,9,12,15,18,21-heptaoxatricosan-1-yl)benzamide was obtained as yellow oil. LC/MS: mass calcd. For C₄₁H₅₇ClN₄O₉: 784.38, found: 785.55 [M+H]⁺.

### Step 3: Synthesis of N-(5-[[2-([2-[(2-[[23-([4-[(2S,4R)-1-acetyl-4-[(4-chlorophenyl)amino]-2-methyl-3,4-dihydro-2H-quinolin-6-yl]phenyl]formamido)-3,6,9,12,15,18,21-heptaoxatricosan-1-yl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 100.00 mg of 4-[(2S,4R)-1-acetyl-4-[(4-chlorophenyl)amino]-2-methyl-3,4-dihydro-2H-quinolin-6-yl]-N-(23-amino-3,6,9,12,15,18,21-heptaoxatricosan-1-yl)benzamide was used, 29.10 mg of N-(5-[[2-([2-[(2-[[23-([4-[(2S,4R)-1-acetyl-4-[(4-chlorophenyl)amino]-2-methyl-3,4-dihydro-2H-quinolin-6-yl]phenyl]formamido)-3,6,9,12,15,18,21-heptaoxatricosan-1-yl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide was obtained as white solid (13.95% yield). HRMS: mass calcd. For C₇₇H₉ClN₁₉O₁₇: 1595.7077, found: 1596.7179 [M+H]⁺.

### N-(5-((3-((2-((1-(4-((2S,4R)-1-acetyl-4-((4-chlorophenyl)amino)-2-methyl-1,2,3,4-tetrahydroquinolin-6-yl)phenyl)-1,30-dioxo-5,8,11,14,17,20,23,26-octaoxa-2,29-diazadotriacontan-32-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 36)

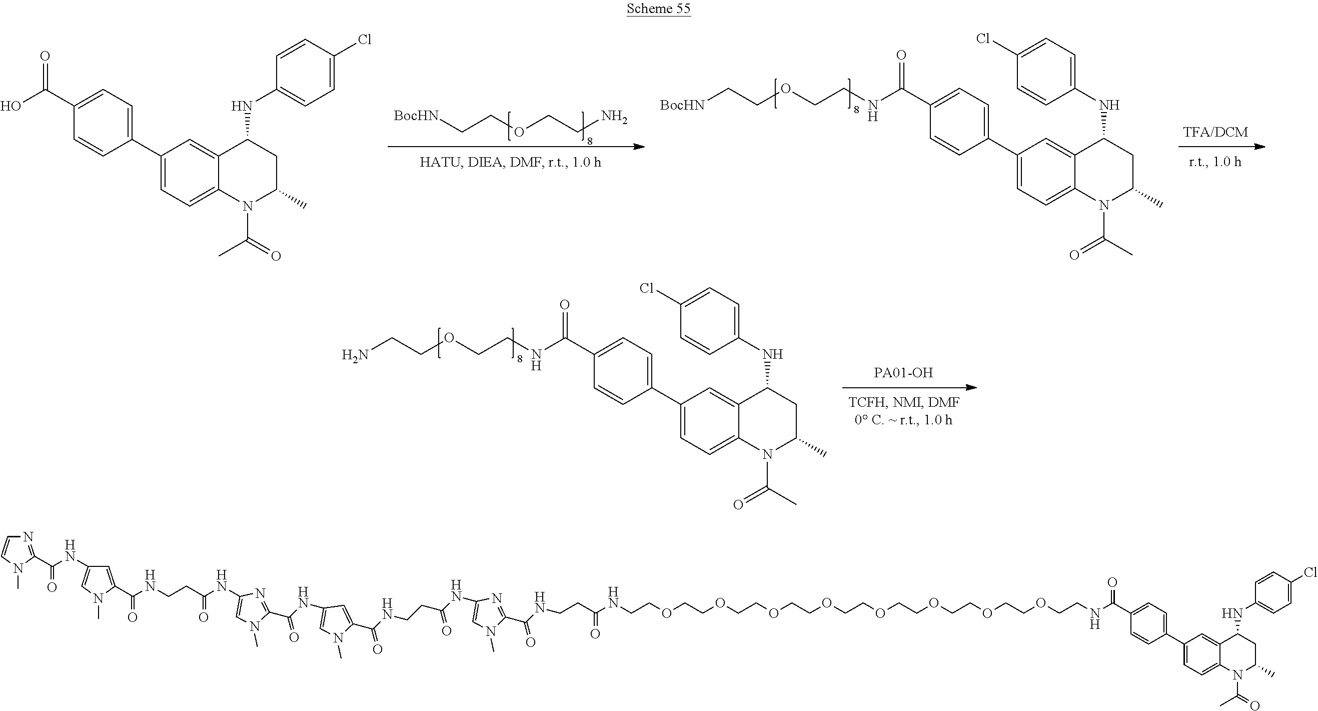

### Step 1: Synthesis of Tert-butyl N-[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamate

To a stirred solution of 4-[(2S,4R)-1-acetyl-4-[(4-chlorophenyl)amino]-2-methyl-3,4-dihydro-2H-quinolin-6-yl]benzoic acid (100.00 mg, 0.23 mmol, 1.00 equiv) in DMF (3.00 mL) was added HATU (131.14 mg, 0.35 mmol, 1.50 equiv), DIEA (89.15 mg, 0.69 mmol, 3.00 equiv) and tert-butyl N-(26-amino-3,6,9,12,15,18,21,24-octaoxahexacosan-1-yl)carbamate (117.87 mg, 0.23 mmol, 1.00 equiv) in portions at 0 degrees C. The resulting mixture was stirred for 1 h at room temperature. The reaction mixture was purified by reverse flash chromatography with the following conditions: column, C18 column; mobile phase, ACN in water (0.05% TFA), 35% to 50% gradient in 20 min; detector, UV 254 nm. The fractions were combined and concentrated. Tert-butyl N-[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamate (170.00 mg, 76.18%) was obtained as yellow oil. LC/MS: mass calcd. For C₄₈H₆₉ClN₄O₁₂: 928.46, found: 929.35 [M+H]⁺.

### Step 2: Synthesis of 4-[(2S,4R)-1-acetyl-4-[(4-chlorophenyl)amino]-2-methyl-3,4-dihydro-2H-quinolin-6-yl]-N-(26-amino-3,6,9,12,15,18,21,24-octaoxahexacosan-1-yl)benzamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 170.00 mg of tert-butyl N-[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamate was used, 170.00 mg crude of 4-[(2S,4R)-1-acetyl-4-[(4-chlorophenyl)amino]-2-methyl-3,4-dihydro-2H-quinolin-6-yl]-N-(26-amino-3,6,9,12,15,18,21,24-octaoxahexacosan-1-yl)benzamide was obtained as yellow oil. LC/MS: mass calcd. For C₄₃H₆₁ClN₄O₁₀: 828.41, found: 829.60 [M+H]⁺.

### Step 3: Synthesis of N-(5-[[2-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 160.00 mg crude of 4-[(2S,4R)-1-acetyl-4-[(4-chlorophenyl)amino]-2-methyl-3,4-dihydro-2H-quinolin-6-yl]-N-(26-amino-3,6,9,12,15,18,21,24-octaoxahexacosan-1-yl)benzamide was used, 27.20 mg of N-(5-[[2-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide was obtained as white solid (8.46% yield). LC/MS: mass calcd. For C₇₉H₁₀₂ClN₁₉O₁₈: 1639.7339, found: 1640.7479 [M+H]⁺.

### (S)—N-(5-((3-((2-((1-(4-(6-(3,5-dimethylisoxazol-4-yl)-1-(1-(pyridin-2-yl)ethyl)-1H-pyrrolo[3,2-b]pyridin-3-yl)phenyl)-1,27-dioxo-5,8,11,14,17,20,23-heptaoxa-2,26-diazanonacosan-29-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 41)

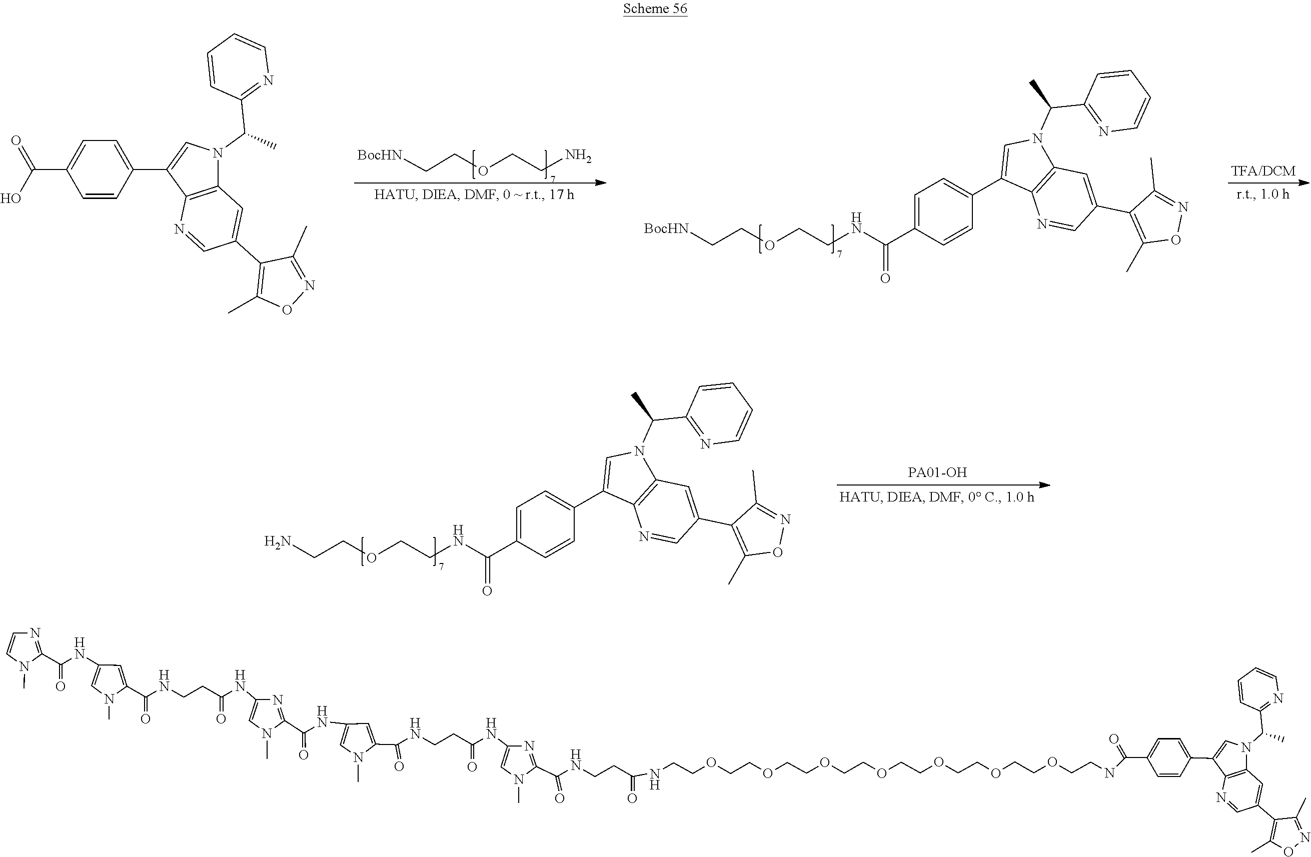

### Step 1: Synthesis of tert-butyl (S)-(1-(4-(6-(3,5-dimethylisoxazol-4-yl)-1-(1-(pyridin-2-yl)ethyl)-1H-pyrrolo[3,2-b]pyridin-3-yl)phenyl)-1-oxo-5,8,11,14,17,20,23-heptaoxa-2-azapentacosan-25-yl)carbamate

The procedure was the same as tert-butyl N-[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamate. 100.00 mg of 4-[6-(3,5-dimethyl-1,2-oxazol-4-yl)-1-[(1S)-1-(pyridin-2-yl)ethyl]pyrrolo[3,2-b]pyridin-3-yl]benzoic acid was used, 329.00 mg crude of tert-butyl (S)-(1-(4-(6-(3,5-dimethylisoxazol-4-yl)-1-(1-(pyridin-2-yl)ethyl)-1H-pyrrolo[3,2-b]pyridin-3-yl)phenyl)-1-oxo-5,8,11,14,17,20,23-heptaoxa-2-azapentacosan-25-yl)carbamate was obtained as yellow oil. LC/MS: mass calcd. For C₄₇H₆₄N₆O₁₁: 888.46, found: 889.70 [M+H]⁺.

### Step 2: Synthesis of (S)—N-(23-amino-3,6,9,12,15,18,21-heptaoxatricosyl)-4-(6-(3,5-dimethylisoxazol-4-yl)-1-(1-(pyridin-2-yl)ethyl)-1H-pyrrolo[3,2-b]pyridin-3-yl)benzamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 300.00 mg of tert-butyl (S)-(1-(4-(6-(3,5-dimethylisoxazol-4-yl)-1-(1-(pyridin-2-yl)ethyl)-1H-pyrrolo[3,2-b]pyridine-3-yl)phenyl)-1-oxo-5,8,11,14,17,20,23-heptaoxa-2-azapentacosan-25-yl)carbamate was used, 300.00 mg crude of (S)—N-(23-amino-3,6,9,12,15,18,21-heptaoxatricosyl)-4-(6-(3,5-dimethylisoxazol-4-yl)-1-(1-(pyridine-2-yl)ethyl)-1H-pyrrolo[3,2-b]pyridin-3-yl)benzamide was obtained as yellow oil. LC/MS: mass calcd. For C₄₂H₅₆N₆O₉: 788.41, found: 789.60 [M+H]⁺.

### Step 3: Synthesis of (S)—N-(5-((3-((2-((1-(4-(6-(3,5-dimethylisoxazol-4-yl)-1-(1-(pyridin-2-yl)ethyl)-1H-pyrrolo[3,2-b]pyridin-3-yl)phenyl)-1,27-dioxo-5,8,11,14,17,20,23-heptaoxa-2,26-diazanonacosan-29-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 107.50 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-yl]formamido)propanoic acid was used, 48.20 mg of (S)—N-(5-((3-((2-((1-(4-(6-(3,5-dimethylisoxazol-4-yl)-1-(1-(pyridin-2-yl)ethyl)-1H-pyrrolo[3,2-b]pyridin-3-yl)phenyl)-1,27-dioxo-5,8,11,14,17,20,23-heptaoxa-2,26-diazanonacosan-29-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (22.63% yield). HRMS: mass calcd. For C₇₈H₉₇N₂₁O₁₇: 1599.7371, found: 1600.7404 [M+H]⁺.

### (S)—N-(5-((3-((2-((1-(4-(6-(3,5-dimethylisoxazol-4-yl)-1-(1-(pyridin-2-yl)ethyl)-1H-pyrrolo[3,2-b]pyridin-3-yl)phenyl)-1,30-dioxo-5,8,11,14,17,20,23,26-octaoxa-2,29-diazadotriacontan-32-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 42)

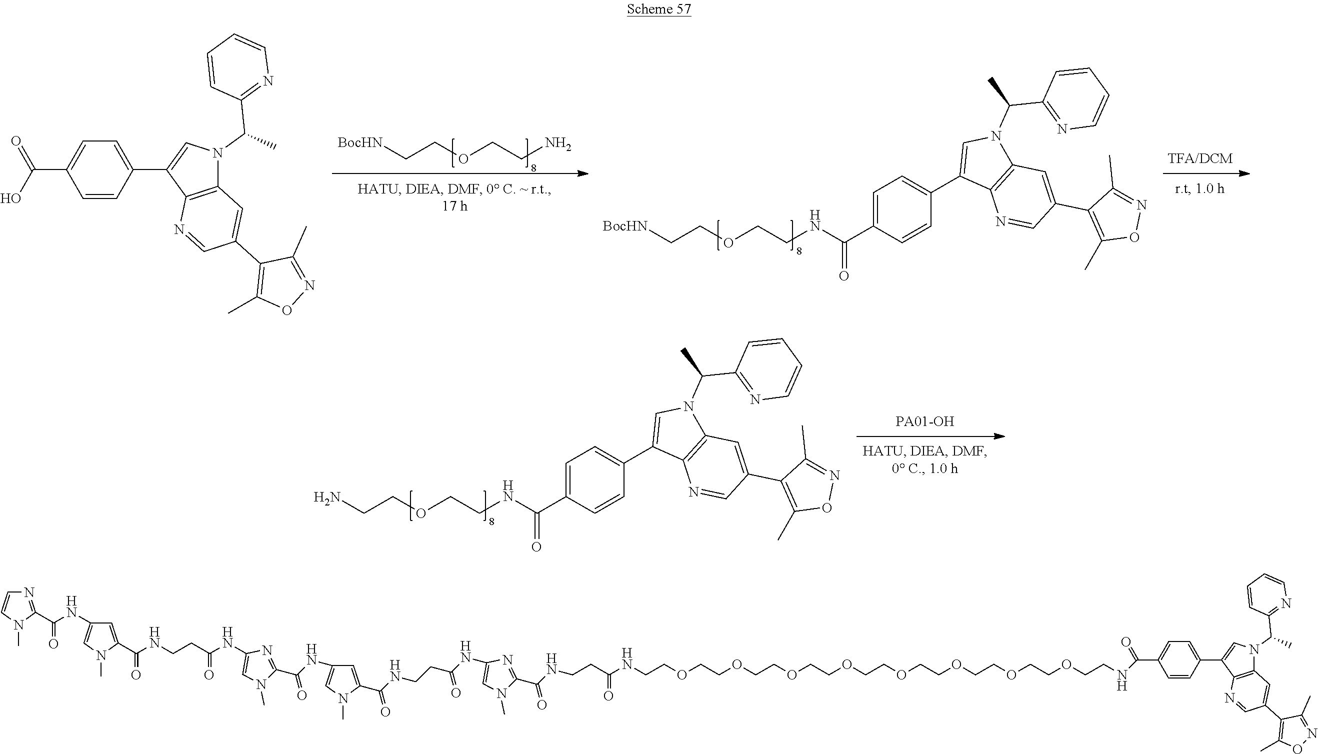

### Step 1: Synthesis of tert-butyl (S)-(1-(4-(6-(3,5-dimethylisoxazol-4-yl)-1-(1-(pyridin-2-yl)ethyl)-1H-pyrrolo[3,2-b]pyridin-3-yl)phenyl)-1-oxo-5,8,11,14,17,20,23,26-octaoxa-2-azaoctacosan-28-yl)carbamate

The procedure was the same as tert-butyl N-[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamate. 100.00 mg of 4-[6-(3,5-dimethyl-1,2-oxazol-4-yl)-1-[(1S)-1-(pyridin-2-yl)ethyl]pyrrolo[3,2-b]pyridin-3-yl]benzoic acid was used, 353.00 mg crude of tert-butyl (S)-(1-(4-(6-(3,5-dimethylisoxazol-4-yl)-1-(1-(pyridin-2-yl)ethyl)-1H-pyrrolo[3,2-b]pyridin-3-yl)phenyl)-1-oxo-5,8,11,14,17,20,23,26-octaoxa-2-azaoctacosan-28-yl)carbamate was obtained as yellow oil. LC/MS: mass calcd. For C₄₉H₆₈N₆O₁₂: 932.49, found: 467.50 [M/2+H]⁺.

### Step 2: Synthesis of (S)—N-(26-amino-3,6,9,12,15,18,21,24-octaoxahexacosyl)-4-(6-(3,5-dimethylisoxazol-4-yl)-1-(1-(pyridin-2-yl)ethyl)-1H-pyrrolo[3,2-b]pyridin-3-yl)benzamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 330.00 mg of tert-butyl (S)-(1-(4-(6-(3,5-dimethylisoxazol-4-yl)-1-(1-(pyridin-2-yl)ethyl)-1H-pyrrolo[3,2-b]pyridin-3-yl)phenyl)-1-oxo-5,8,11,14,17,20,23,26-octaoxa-2-azaoctacosan-28-yl)carbamate was used, 330.00 crude of (S)—N-(26-amino-3,6,9,12,15,18,21,24-octaoxahexacosyl)-4-(6-(3,5-dimethylisoxazol-4-yl)-1-(1-(pyridin-2-yl)ethyl)-1H-pyrrolo[3,2-b]pyridin-3-yl)benzamide was obtained. LC/MS: mass calcd. For C₄₄H₆₀N₆O₁₀: 832.44, found: 833.60 [M+H]⁺.

### Step 3: Synthesis of (S)—N-(5-((3-((2-((1-(4-(6-(3,5-dimethylisoxazol-4-yl)-1-(1-(pyridin-2-yl)ethyl)-1H-pyrrolo[3,2-b]pyridin-3-yl)phenyl)-1,30-dioxo-5,8,11,14,17,20,23,26-octaoxa-2,29-diazadotriacontan-32-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 100.00 mg of 3-([1-methyl-4-[3-([1-methyl-4-[1-methyl-4-(3-[[1-methyl-4-(1-methylimidazole-2-amido) pyrrol-2-yl]formamido]propanamido)imidazole-2-amido]pyrrol-2-yl]formamido)propanamido]imidazol-2-yl]formamido)propanoic acid was used, 40.5 mg of (S)—N-(5-((3-((2-((1-(4-(6-(3,5-dimethylisoxazol-4-yl)-1-(1-(pyridin-2-yl)ethyl)-1H-pyrrolo[3,2-b]pyridin-3-yl)phenyl)-1,30-dioxo-5,8,11,14,17,20,23,26-octaoxa-2,29-diazadotriacontan-32-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (19.97% yield). HRMS: mass calcd. For C₈₀H₁₀₁N₂₁O₁₈: 1643.7633, found: 1644.7672 [M+H]⁺.

### N-(5-[[2-([2-[(2-[[23-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15,18,21-heptaoxatricosan-1-yl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide (Compound 47)

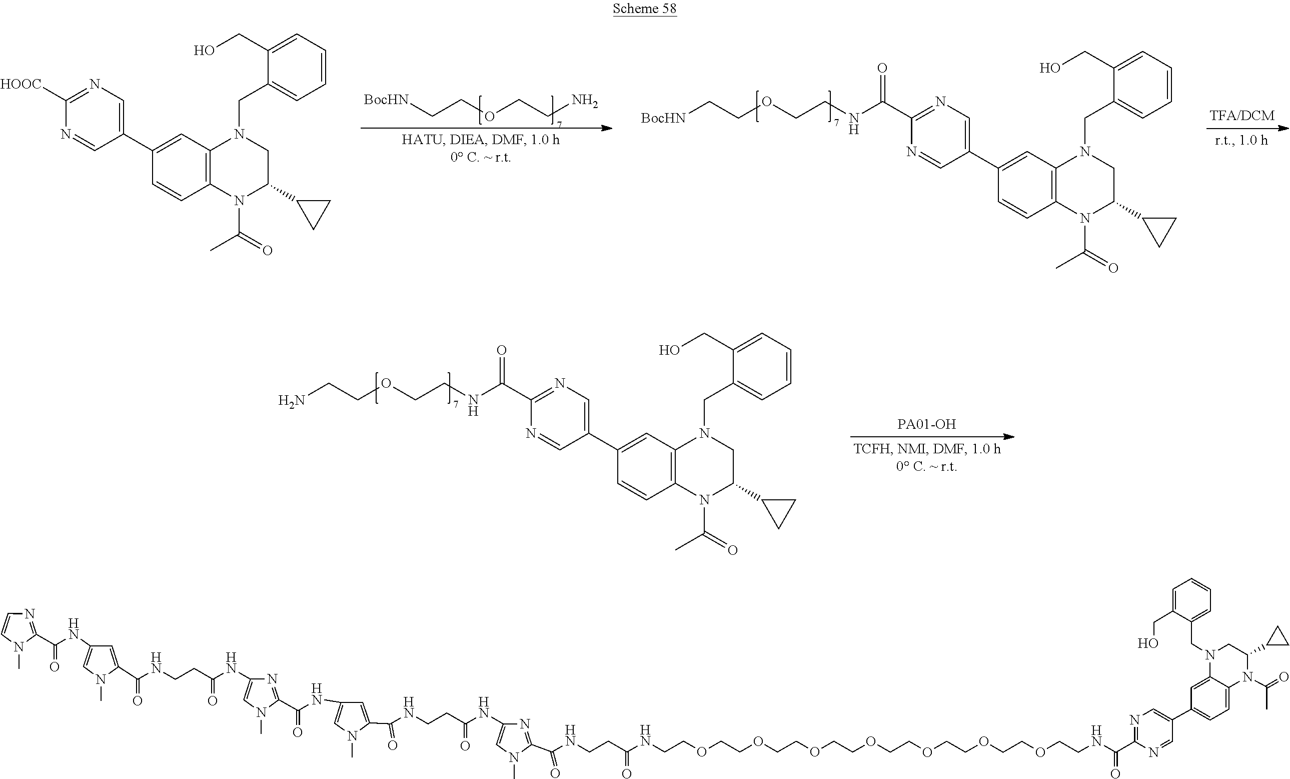

### Step 1: Synthesis of tert-butyl N-[23-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15,18,21-heptaoxatricosan-1-yl]carbamate

The procedure was the same as tert-butyl N-[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamate. 60.00 mg of 5-[(2S)-1-acetyl-2-cyclopropyl-4-[[2-(hydroxymethyl)phenyl]methyl]-2,3-dihydroquinoxalin-6-yl]pyrimidine-2-carboxylic acid was used, 90.00 mg of Tert-butyl N-[23-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15,18,21-heptaoxatricosan-1-yl]carbamate was obtained as yellow oil (68.09% yield). LC/MS: mass calcd. For C₄₇H₆₈N₆O₁₂: 908.49, found: 909.45 [M+H]⁺.

### Step 2: Synthesis of 5-[(2S)-1-acetyl-2-cyclopropyl-4-[[2-(hydroxymethyl)phenyl] methyl]-2,3-dihydroquinoxalin-6-yl]-N-(23-amino-3,6,9,12,15,18,21-heptaoxatricosan-1-yl)pyrimidine-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 90.00 mg of tert-butyl N-[23-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15,18,21-heptaoxatricosan-1-yl]carbamate was used, 90.00 mg crude of 5-[(2S)-1-acetyl-2-cyclopropyl-4-[[2-(hydroxymethyl)phenyl]methyl]-2,3-dihydroquinoxalin-6-yl]-N-(23-amino-3,6,9,12,15,18,21-heptaoxatricosan-1-yl)pyrimidine-2-carboxamide was obtained as yellow oil. LC/MS: mass calcd. For C₄₂H₆₀N₆O₁₀: 808.44, found: 809.35 [M+H]⁺.

### Step 3: Synthesis of N-(5-[[2-([2-[(2-[[23-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15,18,21-heptaoxatricosan-1-yl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 85.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-yl]formamido)propanoic acid was used, 11.20 mg of N-(5-[[2-([2-[(2-[[23-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15,18,21-heptaoxatricosan-1-yl]carbamoyl]ethyl)carbamoyl]-1-methylimidazol-4-yl]carbamoyl)ethyl] carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide was obtained as yellow solid (6.49% yield). HRMS: mass calcd. For C₇₈H₁₀₁N₂₁O₁₈: 1619.76, found: 1620.7650 [M+H]⁺.

### N-(5-[[2-([2-[(2-[[26-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15,18,21,24-octaoxahexacosan-1-yl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide (Compound 48)

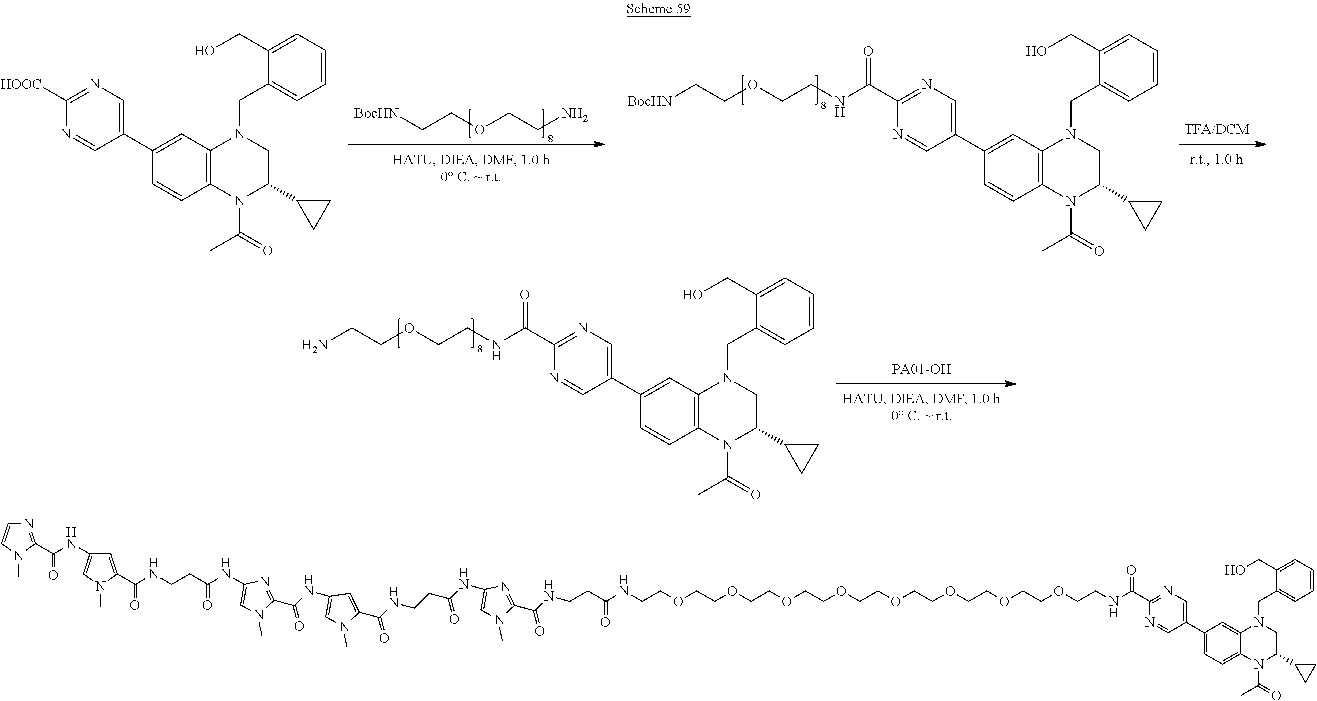

### Step 1: Synthesis of Tert-butyl N-[26-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15,18,21,24-octaoxahexacosan-1-yl]carbamate

The procedure was the same as tert-butyl N-[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamate. 50.00 mg of 5-[(2S)-1-acetyl-2-cyclopropyl-4-[[2-(hydroxymethyl)phenyl]methyl]-2,3-dihydroquinoxalin-6-yl]pyrimidine-2-carboxylic acid was used, 101.00 mg of tert-butyl N-[26-([5-[(2S)-1-acetyl-2-cyclopropyl-4-[[2-(hydroxymethyl)phenyl] methyl]-2,3-dihydroquinoxalin-6-yl]pyrimidin-2-yl]formamido)-3,6,9,12,15,18,21,24-octaoxahexacosan-1-yl]carbamate was obtained as yellow oil (87.46% yield). LC/MS: mass calcd. For C₄₉H₇₂N₆O₁₃: 952.52, found: 953.40 [M+H]⁺.

### Step 2: Synthesis of 5-[(2S)-1-acetyl-2-cyclopropyl-4-[[2-(hydroxymethyl)phenyl]methyl]-2,3-dihydroquinoxalin-6-yl]-N-(26-amino-3,6,9,12,15,18,21,24-octaoxahexacosan-1-yl)pyrimidine-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 100.00 mg of tert-butyl N-[26-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15,18,21,24-octaoxahexacosan-1-yl]carbamate was used, 100.00 mg crude of 5-[(2S)-1-acetyl-2-cyclopropyl-4-[[2-(hydroxymethyl)phenyl]methyl]-2,3-dihydroquinoxalin-6-yl]-N-(26-amino-3,6,9,12,15,18,21,24-octaoxahexacosan-1-yl)pyrimidine-2-carboxamide was obtained as yellow oil. LC/MS: mass calcd. For C₄₄H₆₄N₆O₁₁: 852.46, found: 853.35 [M+H]⁺.

### Step 3: Synthesis of N-(5-[[2-([2-[(2-[[26-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15,18,21,24-octaoxahexacosan-1-yl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 95.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido] imidazole-2-yl]formamido)propanoic acid was used, 20.00 mg of N-(5-[[2-([2-[(2-[[26-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15,18,21,24-octaoxahexacosan-1-yl]carbamoyl]ethyl)carbamoyl]-1-methylimidazol-4-yl]carbamoyl)ethyl]carbamoyl]-1-methylpyrrol-3yl)-1-methyl-4-(3-[[1-methyl-4-(1-methylimidazole-2-amido)pyrrol-2-yl]formamido]propanamido)imidazole-2-carboxamide was obtained as white solid (10.15% yield). HRMS: mass calcd. For C₈₀H₁₀₅N₂₁O₁₉: 1663.79, found: 1664.7964 [M+H]⁺.

Type 3 Reactions in the Synthesis of Transcription Modulator Molecules

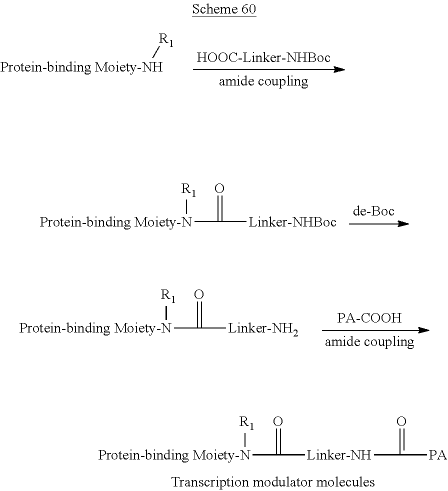

### N-[5-[(2-[[2-([2-[(23-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-23-oxo-3,6,9,12,15,18,21-heptaoxatricosan-1-yl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido) imidazole-2-carboxamide (Compound 43)

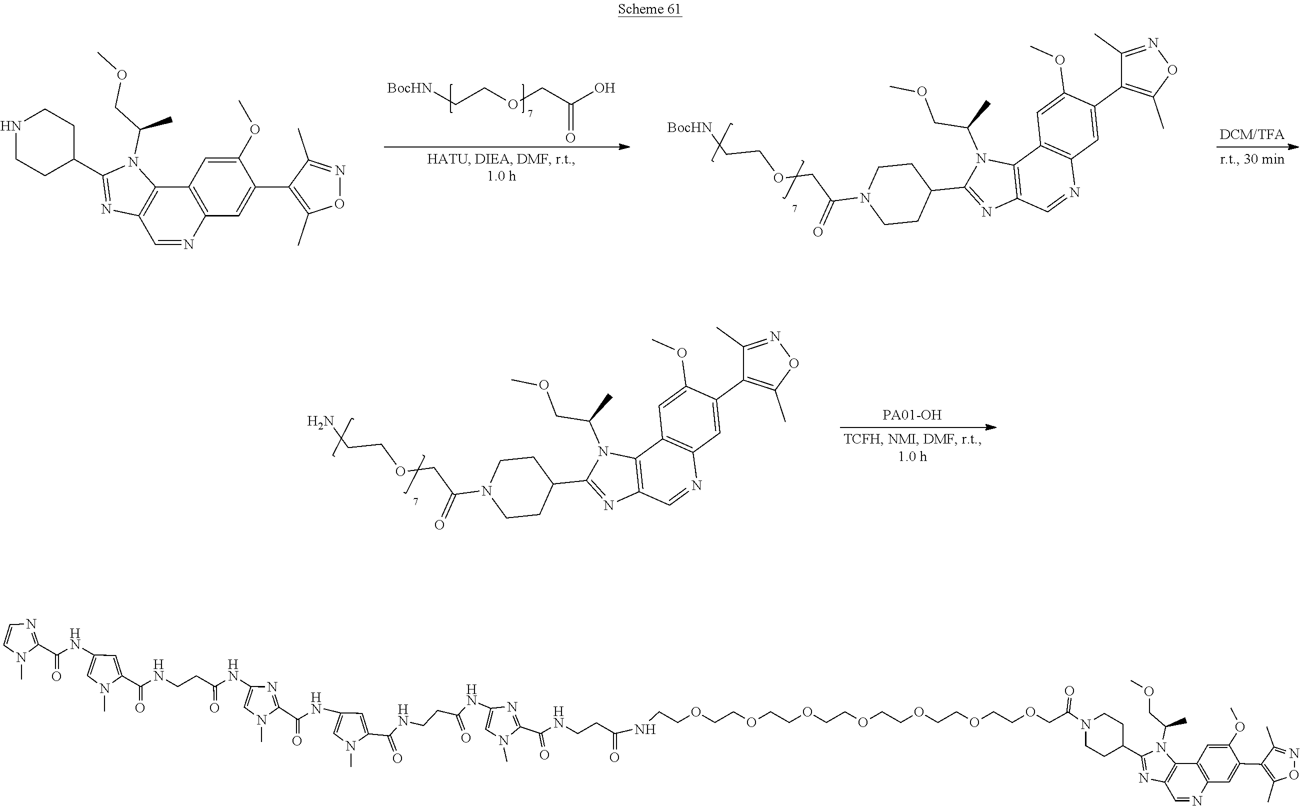

### Step 1: Synthesis of tert-butyl N-(23-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-23-oxo-3,6,9,12,15,18,21-heptaoxatricosan-1-yl)carbamate

To a stirred solution of 23-[(tert-butoxycarbonyl)amino]-3,6,9,12,15,18,21-heptaoxatricosanoic acid (107.56 mg, 0.22 mmol, 1.00 equiv) in DMF (3.00 mL) was added HATU (126.87 mg, 0.33 mmol, 1.50 equiv), DIEA (86.25 mg, 0.67 mmol, 3.00 equiv) and 4-[7-(3,5-dimethyl-1,2-oxazol-4-yl)-8-methoxy-1-[(2R)-1-methoxypropan-2-yl]imidazo [4,5-c]quinolin-2-yl]piperidine (100.00 mg, 0.22 mmol, 1.00 equiv) in portions at 0 degrees C. The resulting mixture was stirred for 1 h at room temperature. The reaction mixture was purified by reverse flash chromatography with the following conditions: column, C18 column; mobile phase, ACN in water(0.05% TFA), 25% to 35% gradient in 30 min; detector, UV 254 nm. The fractions were combined and concentrated. Tert-butyl N-(23-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-23-oxo-3,6,9,12,15,18,21-heptaoxatricosan-1-yl)carbamate (140.00 mg, 61.53%) was obtained as yellow oil. LC/MS: mass calcd. For C₄₆H₇₀N₆O₁₃: 914.50, found: 915.45 [M+H]⁺.

### Step 2: 23-amino-1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-3,6,9,12,15,18,21-heptaoxatricosan-1-one

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 140.00 mg of tert-butyl N-(23-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-23-oxo-3,6,9,12,15,18,21-heptaoxatricosan-1-yl)carbamate was used, 140.00 mg crude of 23-amino-1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-3,6,9,12,15,18,21-heptaoxatricosan-1-one was obtained as yellow oil. LC/MS: mass calcd. For C₄₁H₆₂N₆O₁₁: 814.45, found: 815.30 [M+H]⁺.

### Step 3: N-[5-[(2-[[2-([2-[(23-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-23-oxo-3,6,9,12,15,18,21-heptaoxatricosan-1-yl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 70.00 mg of 23-amino-1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-3,6,9,12,15,18,21-heptaoxatricosan-1-one was used, 13.80 mg of N-[5-[(2-[[2-([2-[(23-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-23-oxo-3,6,9,12,15,18,21-heptaoxatricosan-1-yl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as white solid (9.64% yield). HRMS: mass calcd. For C₇₇H₁₀₃N₂₁O₁₉: 1625.7739, found: 1626.7821 [M+H]⁺.

### (S)—N-(5-((3-((2-((1-(4-(1-acetyl-2-cyclopropyl-4-(2-(hydroxymethyl)benzyl)-1,2,3,4-tetrahydroquinoxalin-6-yl)-3,6-dihydropyridin-1(2H)-yl)-1,25-dioxo-3,6,9,12,15,18,21-heptaoxa-24-azaheptacosan-27-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 45)

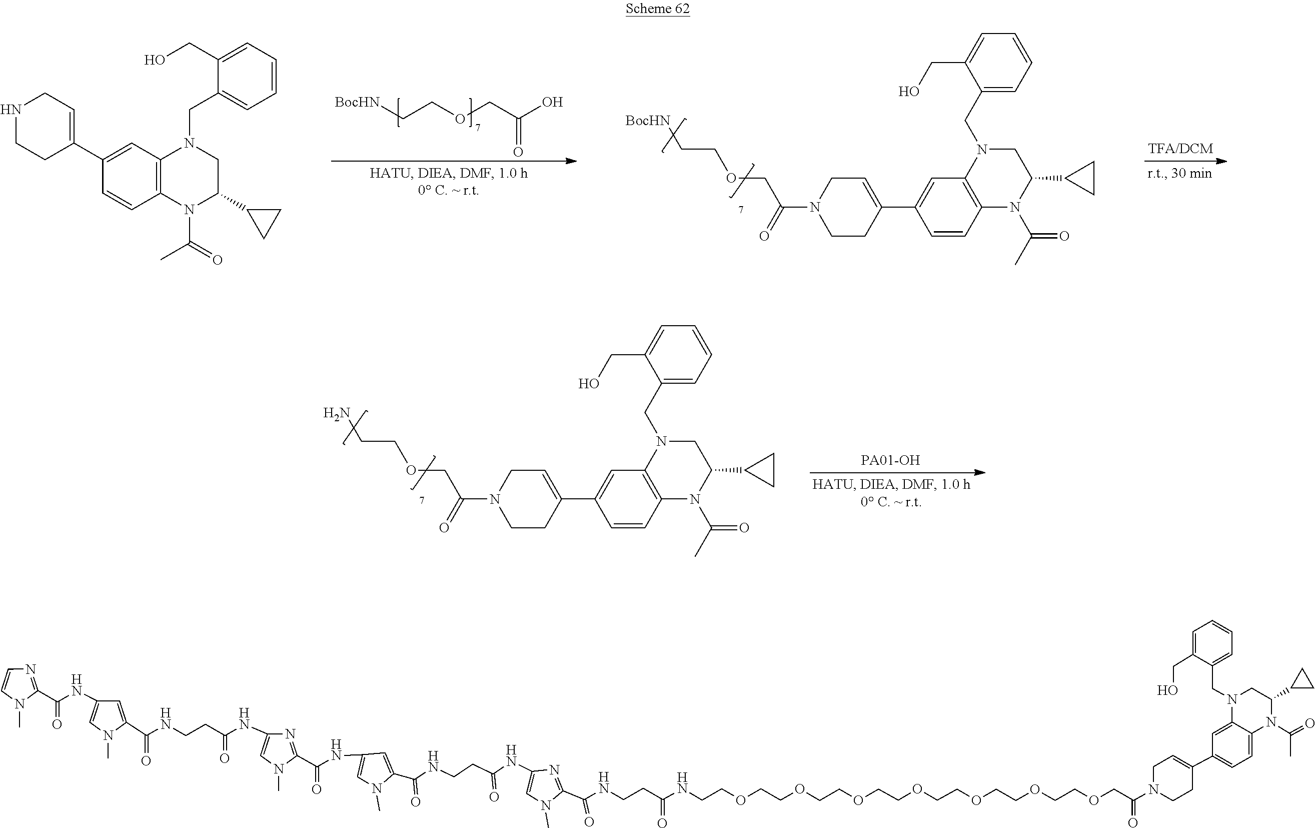

### Step 1: Synthesis of Tert-butyl N-(23-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-23-oxo-3,6,9,12,15,18,21-heptaoxatricosan-1-yl)carbamate

The procedure was the same as tert-butyl N-(23-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-23-oxo-3,6,9,12,15,18,21-heptaoxatricosan-1-yl)carbamate. 60.00 mg of 1-[(2S)-2-cyclopropyl-4-[[2-(hydroxymethyl)phenyl]methyl]-6-(1,2,3,6-tetrahydropyridin-4-yl)-2,3-dihydroquinoxalin-1-yl]ethanone was used, 113.00 mg of tert-butyl N-(23-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-23-oxo-3,6,9,12,15,18,21-heptaoxatricosan-1-yl)carbamate was obtained as yellow oil (82.82% yield). LC/MS: mass calcd. For C₄₇H₇₀N₄O₁₂: 882.50, found: 883.70 [M+H]⁺.

### Step 2: Synthesis of 1-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-23-amino-3,6,9,12,15,18,21-heptaoxatricosan-1-one

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 100.00 mg of tert-butyl N-(23-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-23-oxo-3,6,9,12,15,18,21-heptaoxatricosan-1-yl)carbamate was used, 146.00 mg crude of 1-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-23-amino-3,6,9,12,15,18,21-heptaoxatricosan-1-one was obtained as yellow oil. LC/MS: mass calcd. For C₄₇H₇₀N₄O₁₂: 782.45, found: 783.60 [M+H]⁺.

### Step 3: Synthesis of (S)—N-(5-((3-((2-((1-(4-(1-acetyl-2-cyclopropyl-4-(2-(hydroxymethyl)benzyl)-1,2,3,4-tetrahydroquinoxalin-6-yl)-3,6-dihydropyridin-1(2H)-yl)-1,25-dioxo-3,6,9,12,15,18,21-heptaoxa-24-azaheptacosan-27-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 100.00 mg of 1-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-23-amino-3,6,9,12,15,18,21-heptaoxatricosan-1-one was used, 13.90 mg of (S)—N-(5-((3-((2-((1-(4-(1-acetyl-2-cyclopropyl-4-(2-(hydroxymethyl)benzyl)-1,2,3,4-tetrahydroquinoxalin-6-yl)-3,6-dihydropyridin-1(2H)-yl)-1,25-dioxo-3,6,9,12,15,18,21-heptaoxa-24-azaheptacosan-27-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (6.82% yield). LC/MS: mass calcd. For C₇₈H₁₀₃N₁₉O₁₈: 1593.77, found: 1594.7822 [M+H]⁺.

### N-[5-[(2-[[2-([2-[(26-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-26-oxo-3,6,9,12,15,18,21,24-octaoxahexacosan-1-yl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide (Compound 46)

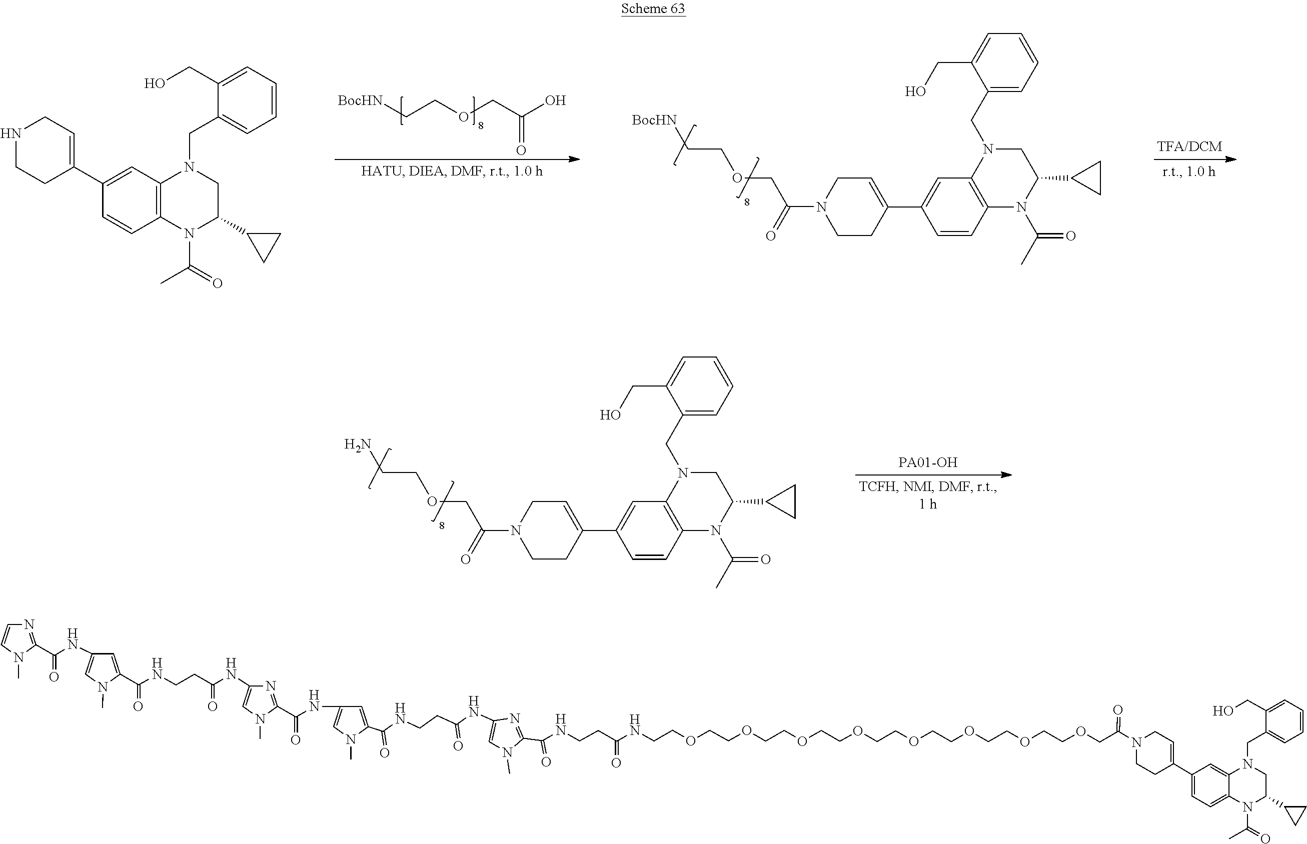

### Step 1: Synthesis of tert-butyl N-(26-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-26-oxo-3,6,9,12,15,18,21,24-octaoxahexacosan-1-yl)carbamate

The procedure was the same as tert-butyl N-(23-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-23-oxo-3,6,9,12,15,18,21-heptaoxatricosan-1-yl)carbamate. 70.44 mg of 1-[(2S)-2-cyclopropyl-4-[[2-(hydroxymethyl)phenyl]methyl]-6-(1,2,3,6-tetrahydropyridin-4-yl)-2,3-dihydroquinoxalin-1-yl]ethanone was used, 110.00 mg of tert-butyl N-(26-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-26-oxo-3,6,9,12,15,18,21,24-octaoxahexacosan-1-yl)carbamate was obtained as yellow oil (70.33% yield). LC/MS: mass calcd. For C₄₉H₇₄N₄O₁₃: 926.53, found: 927.75 [M+H]⁺.

### Step 2: Synthesis of 1-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-26-amino-3,6,9,12,15,18,21,24-octaoxahexacosan-1-one

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 100.00 mg of tert-butyl N-(26-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-26-oxo-3,6,9,12,15,18,21,24-octaoxahexacosan-1-yl)carbamate was used, 100.00 mg crude of 1-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-26-amino-3,6,9,12,15,18,21,24-octaoxahexacosan-1-one was obtained as yellow oil. LC/MS: mass calcd. For C₄₄H₆₆N₄O₁₁: 826.47, found: 827.70 [M+H]⁺.

### Step 3: Synthesis of N-[5-[(2-[[2-([2-[(26-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-26-oxo-3,6,9,12,15,18,21,24-octaoxahexacosan-1-yl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 100.00 mg of 1-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-26-amino-3,6,9,12,15,18,21,24-octaoxahexacosan-1-one was used, 22.10 mg of N-[5-[(2-[[2-([2-[(26-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-26-oxo-3,6,9,12,15,18,21,24-octaoxahexacosan-1-yl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as white solid (10.96% yield). HRMS: mass calcd. For C₈₀H₁₀₇N₁₉O₁₉: 1637.7991, found: 1638.8066 [M+H]⁺.

Type 5 Reactions in the Synthesis of Transcription Modulator Molecules

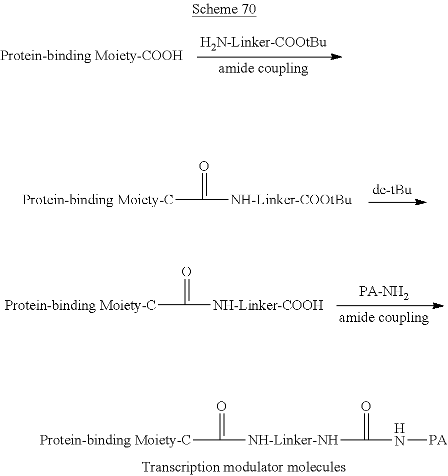

### (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-2,24,34-trioxo-6,9,12,15,18,21-hexaoxa-3,25,29,33-tetraazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 1)

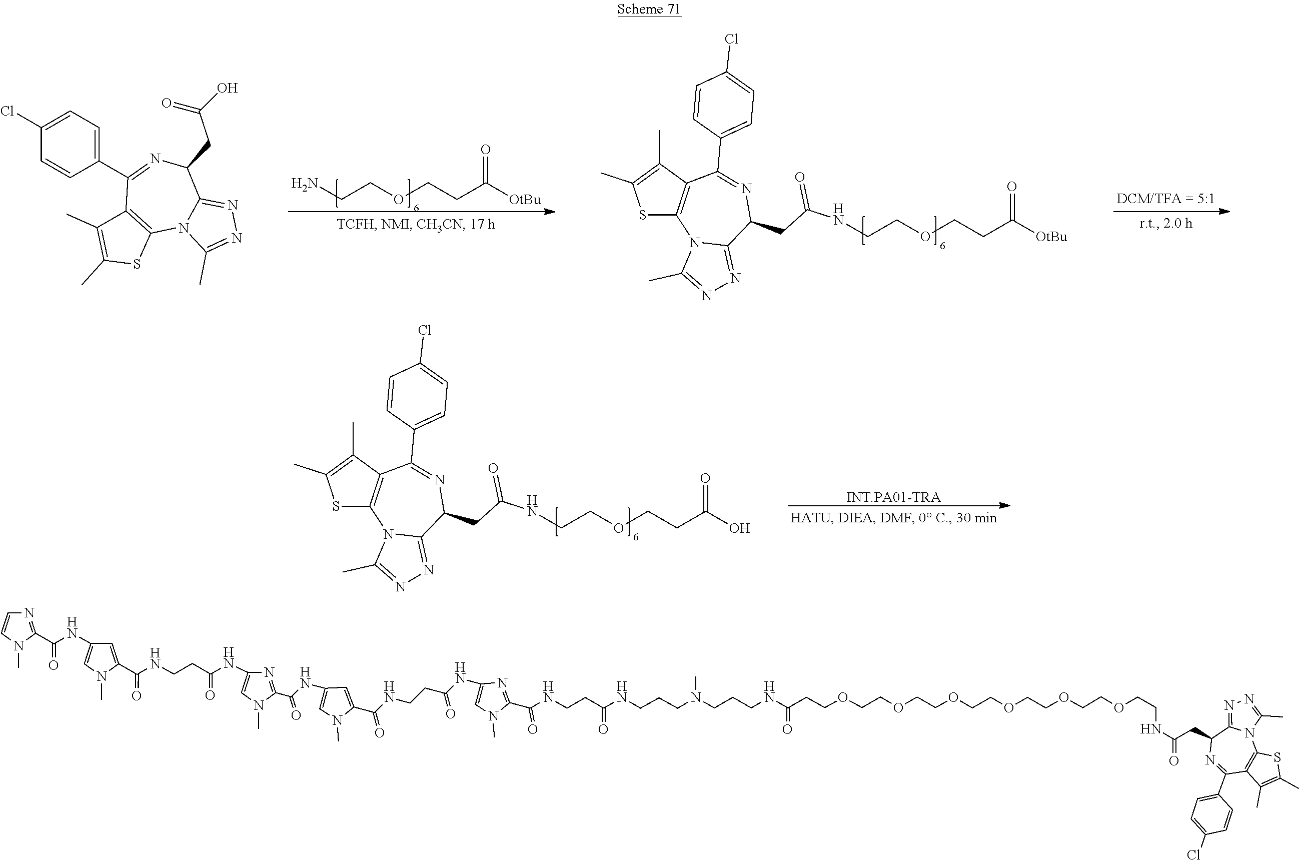

### Step 1: Synthesis of tert-butyl (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oate

The procedure was the same as tert-butyl N-[23-([4-[(2S,4R)-1-acetyl-4-[(4-chlorophenyl)amino]-2-methyl-3,4-dihydro-2H-quinolin-6-yl]phenyl]formamido)-3,6,9,12,15,18,21-heptaoxatricosan-1-yl]carbamate. 100.00 mg of (S)-2-(4-(4-chlorophenyl)-2,3,9-trimethyl-6H-thieno[3,2-f][1,2,4]triazolo[4,3-a][1,4]diazepin-6-yl)acetic acid was used, 120.00 mg of tert-butyl (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oate was obtained as yellow oil (55.71% yield). LC/MS: mass calcd. For C₃₈H₅₄ClN₅O₉S: 791.33, found: 792.30 [M+H]⁺.

### Step 2: Synthesis of (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid

Into a 50 mL flask was added tert-butyl (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oate (120.00 mg, 0.15 mmol, 1.00 equiv), DCM (2.50 mL) and TFA (0.50 mL). The mixture was stirred at room temperature for 2.0 h. LCMS showed the reaction complete. The reaction was concentrated. (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid (120.00 mg, crude) was obtained as yellow oil. LC/MS: mass calcd. For C₃₄H₄₆ClN₅O₉S: 735.27, found: 736.25 [M+H]⁺.

### Step 3: Synthesis of (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-2,24,34-trioxo-6,9,12,15,18,21-hexaoxa-3,25,29,33-tetraazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

To a solution of (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid (75.00 mg, 0.10 mmol, 1.00 equiv) in DMF (2.00 mL) was added HATU (58.12 mg, 0.15 mmol, 1.50 equiv), DIEA (39.51 mg, 0.31 mmol, 3.00 equiv) and N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA)(106.14 mg, 0.11 mmol, 1.05 equiv) at 0 degree C. The mixture was stirred for 30 mins in the ice bath. The solution was filtered and purified with Prep-HPLC with the following conditions: Column: XSelect CSH Prep C18 OBD Column, 19*250 mm, 5 um; Mobile Phase A: Water (10 MMOL/L NH₄HCO₃), Mobile Phase B: ACN; Flow rate: 25 mL/min; Gradient: 30 B to 50 B in 11 min; 220 nm; RT1: 9.98. The fractions were lyophilized directly. (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-2,24,34-trioxo-6,9,12,15,18,21-hexaoxa-3,25,29,33-tetraazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (37.5 mg, 25.3%) was obtained as white solid. HRMS: mass calcd. For C₇₇H₁₀₄ClN₂₃O₁₆S: 1673.7441, found: 1674.7498 [M+H]⁺.

### N-(5-[[2-([2-[(2-[[3-([3-[1-([4-[6-(3,5-dimethyl-1,2-oxazol-4-yl)-1-[(1S)-1-(pyridin-2-yl)ethyl]pyrrolo[3,2-b]pyridin-3-yl]phenyl]formamido)-3,6,9,12,15-pentaoxaoctadecan-18-amido]propyl](methyl)amino)propyl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide (Compound 8)

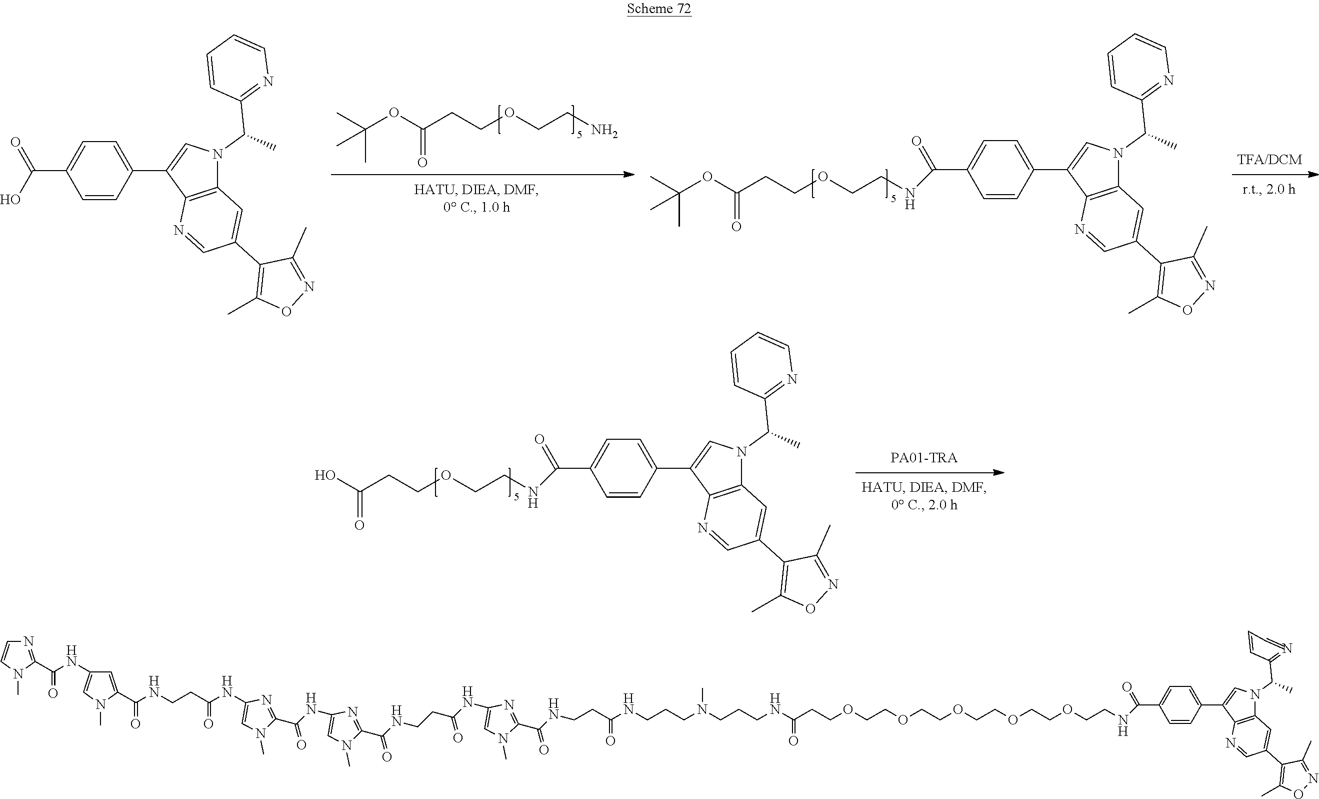

### Step 1: Synthesis of tert-butyl 1-([4-[6-(3,5-dimethyl-1,2-oxazol-4-yl)-1-[(1S)-1-(pyridin-2-yl)ethyl]pyrrolo[3,2-b]pyridin-3-yl]phenyl]formamido)-3,6,9,12,15-pentaoxaoctadecan-18-oate

The procedure was the same as tert-butyl N-[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamate. 150.00 mg of 4-[6-(3,5-dimethyl-1,2-oxazol-4-yl)-1-[(1S)-1-(pyridin-2-yl)ethyl]pyrrolo[3,2-b]pyridin-3-yl]benzoic acid was used, 350.00 mg crude of tert-butyl 1-([4-[6-(3,5-dimethyl-1,2-oxazol-4-yl)-1-[(1S)-1-(pyridin-2-yl)ethyl]pyrrolo[3,2-b]pyridin-3-yl]phenyl]formamido)-3,6,9,12,15-pentaoxaoctadecan-18-oate was obtained as yellow oil. LCMS: mass calcd. For C₄₃H₅₅N₅O₉:785.40, found: 786.55 [M+H]⁺.

### Step 2: Synthesis of 1-([4-[6-(3,5-dimethyl-1,2-oxazol-4-yl)-1-[(1S)-1-(pyridin-2-yl)ethyl]pyrrolo[3,2-b]pyridin-3-yl]phenyl]formamido)-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 350.00 mg crude of tert-butyl 1-([4-[6-(3,5-dimethyl-1,2-oxazol-4-yl)-1-[(1S)-1-(pyridin-2-yl)ethyl]pyrrolo[3,2-b]pyridin-3-yl]phenyl]formamido)-3,6,9,12,15-pentaoxaoctadecan-18-oate was used, 350.00 mg crude of 1-([4-[6-(3,5-dimethyl-1,2-oxazol-4-yl)-1-[(1S)-1-(pyridin-2-yl)ethyl]pyrrolo[3,2-b]pyridin-3-yl]phenyl]formamido)-3,6,9,12,15-pentaoxaoctadecan-18-oic acid was obtained as yellow oil. LCMS: mass calcd. For C₃₉H₄₇N₅O₉:729.34, found: 730.45 [M+H]⁺.

### Step 3: Synthesis of N-(5-[[2-([2-[(2-[[3-([3-[1-([4-[6-(3,5-dimethyl-1,2-oxazol-4-yl)-1-[(1S)-1-(pyridin-2-yl)ethyl]pyrrolo[3,2-b]pyridin-3-yl]phenyl]formamido)-3,6,9,12,15-pentaoxaoctadecan-18-amido]propyl](methyl)amino)propyl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide

The procedure was the same as (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-2,24,34-trioxo-6,9,12,15,18,21-hexaoxa-3,25,29,33-tetraazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 150.00 mg of 1-([4-[6-(3,5-dimethyl-1,2-oxazol-4-yl)-1-[(1S)-1-(pyridin-2-yl)ethyl]pyrrolo[3,2-b]pyridin-3-yl]phenyl]formamido)-3,6,9,12,15-pentaoxaoctadecan-18-oic acid was used, 34.3 mg of N-(5-[[2-([2-[(2-[[3-([3-[1-([4-[6-(3,5-dimethyl-1,2-oxazol-4-yl)-1-[(1S)-1-(pyridin-2-yl)ethyl]pyrrolo[3,2-b]pyridin-3-yl]phenyl]formamido)-3,6,9,12,15-pentaoxaoctadecan-18-amido]propyl](methyl)amino)propyl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide was obtained as white solid (9.79% yield). HRMS: mass calcd. For C₈₂H₁₀₅N₂₃O₁₆: 1667.8110, found: 1668.8252 [M+H]⁺.

### N-(5-[[2-([2-[(2-[[3-([3-[1-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15-pentaoxaoctadecan-18-amido]propyl](methyl)amino)propyl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide (Compound 30)

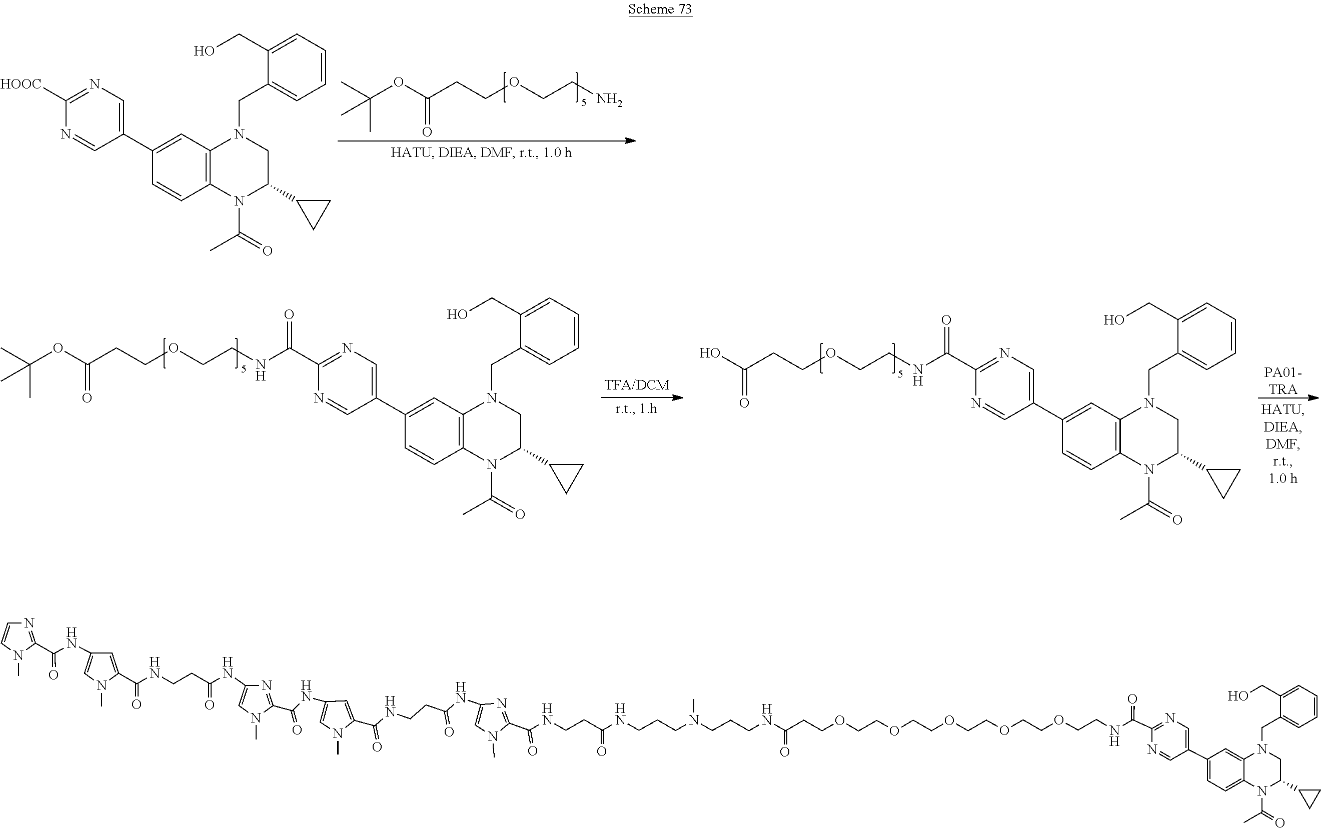

### Step 1: Synthesis of tert-butyl 1-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15-pentaoxaoctadecan-18-oate

The procedure was the same as tert-butyl N-[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamate. 130.00 mg of 5-[(2S)-1-acetyl-2-cyclopropyl-4-[[2-(hydroxymethyl)phenyl]methyl]-2,3-dihydroquinoxalin-6-yl]pyrimidine-2-carboxylic acid was used, 80.00 mg of tert-butyl 1-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15-pentaoxaoctadecan-18-oate was obtained as a yellow oil (27.31% yield). LC/MS: mass calcd. For C₄₃H₅₉N₅O₁₀: 805.43, found: 806.40 [M+H]⁺.

### Step 2: Synthesis of 1-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 80.00 mg of tert-butyl 1-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15-pentaoxaoctadecan-18-oate was used, 80.00 mg crude of 1-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15-pentaoxaoctadecan-18-oic acid was obtained as yellow oil. LC/MS: mass calcd. For C₃₉H₅₁N₅O₁₀: 749.36, found: 750.55 [M+H]⁺.

### Step 3: Synthesis of N-(5-[[2-([2-[(2-[[3-([3-[1-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15-pentaoxaoctadecan-18-amido]propyl](methyl)amino)propyl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide

The procedure was the same as (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-2,24,34-trioxo-6,9,12,15,18,21-hexaoxa-3,25,29,33-tetraazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 75.00 mg of 1-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15-pentaoxaoctadecan-18-oic acid was used, 24.50 mg of N-(5-[[2-([2-[(2-[[3-([3-[1-([5-[(2S)-1-acetyl-2-cyclopropyl-4-[[2-(hydroxymethyl)phenyl]methyl]-2,3-dihydroquinoxalin-6-yl]pyrimidin-2-yl]formamido)-3,6,9,12,15-pentaoxaoctadecan-18-amido]propyl](methyl) amino)propyl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido] propanamido)imidazole-2-carboxamide was obtained as white solid (13.71% yield). HRMS: mass calcd. For C₈₂H₁₀₉N₂₃O₁₇: 1687.8372, found: 1688.8383 [M+H]⁺.

### N-(5-[[2-([2-[(2-[[3-([3-[1-([4-[(2S,4R)-1-acetyl-4-[(4-chlorophenyl)amino]-2-methyl-3,4-dihydro-2H-quinolin-6-yl]phenyl]formamido)-3,6,9,12,15-pentaoxaoctadecan-18-amido]propyl](methyl)amino)propyl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido] propanamido)imidazole-2-carboxamide (Compound 31)

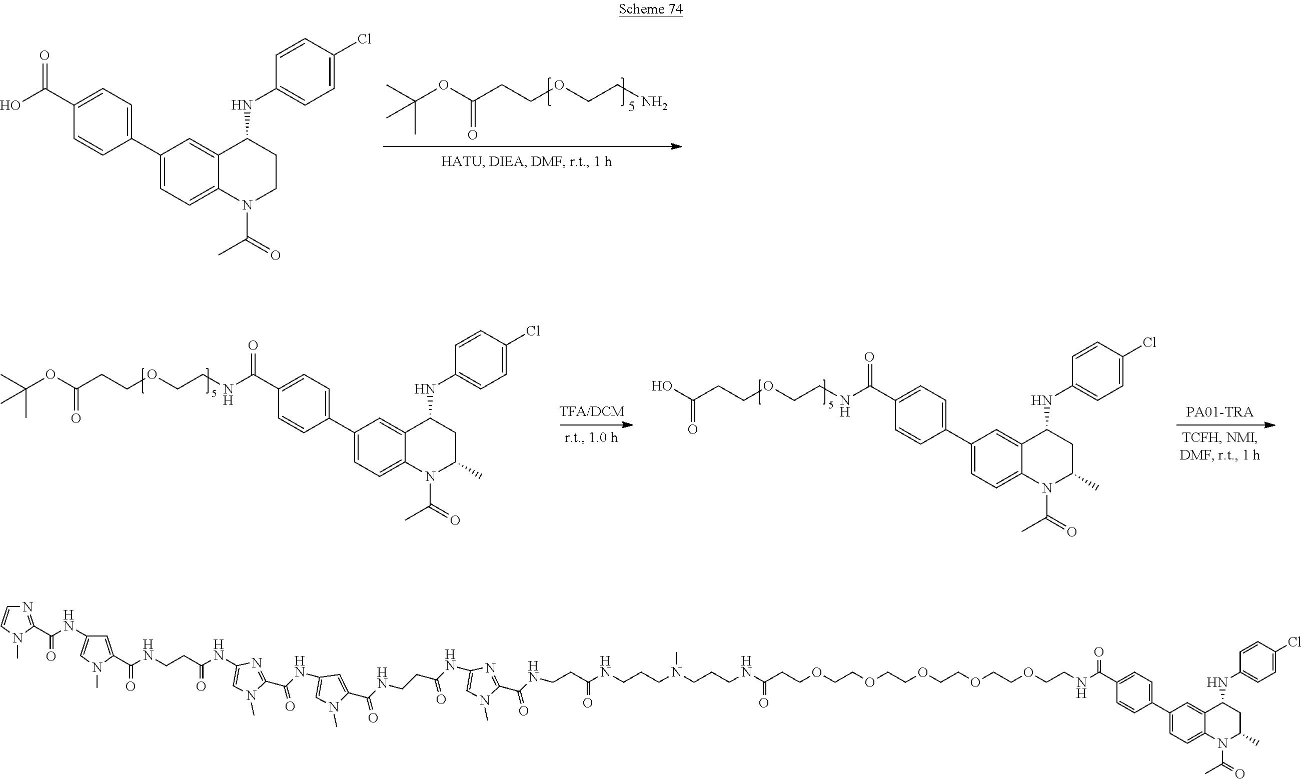

### Step 1: Synthesis of tert-butyl 1-([4-[(2S,4R)-1-acetyl-4-[(4-chlorophenyl)amino]-2-methyl-3,4-dihydro-2H-quinolin-6-yl]phenyl]formamido)-3,6,9,12,15-pentaoxaoctadecan-18-oate

The procedure was the same as tert-butyl N-[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamate. 120.00 mg of 4-[(2S,4R)-1-acetyl-4-[(4-chlorophenyl)amino]-2-methyl-3,4-dihydro-2H-quinolin-6-yl]benzoic acid was used, 150.00 mg of tert-butyl 1-([4-[(2S,4R)-1-acetyl-4-[(4-chlorophenyl)amino]-2-methyl-3,4-dihydro-2H-quinolin-6-yl]phenyl]formamido)-3,6,9,12,15-pentaoxaoctadecan-18-oate was obtained as yellow oil (63.99% yield). LC/MS: mass calcd. For C₄₂H₅₆ClN₃O₉: 781.37, found: 804.20 [M+Na]⁺.

### Step 2: Synthesis of 1-([4-[(2S,4R)-1-acetyl-4-[(4-chlorophenyl)amino]-2-methyl-3,4-dihydro-2H-quinolin-6-yl]phenyl]formamido)-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 150.00 mg of tert-butyl 1-([4-[(2S,4R)-1-acetyl-4-[(4-chlorophenyl)amino]-2-methyl-3,4-dihydro-2H-quinolin-6-yl]phenyl]formamido)-3,6,9,12,15-pentaoxaoctadecan-18-oate was used, 150.00 mg crude of 1-([4-[(2S,4R)-1-acetyl-4-[(4-chlorophenyl)amino]-2-methyl-3,4-dihydro-2H-quinolin-6-yl]phenyl]formamido)-3,6,9,12,15-pentaoxaoctadecan-18-oic acid was obtained as yellow oil. LC/MS: mass calcd. For C₃₈H₄₈ClN₃O₉: 725.31, found: 726.45 [M+H]⁺.

### Step 3: Synthesis of N-(5-[[2-([2-[(2-[[3-([3-[1-([4-[(2S,4R)-1-acetyl-4-[(4-chlorophenyl)amino]-2-methyl-3,4-dihydro-2H-quinolin-6-yl]phenyl]formamido)-3,6,9,12,15-pentaoxaoctadecan-18-amido]propyl](methyl)amino)propyl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido] propanamido)imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 140.00 mg of 1-([4-[(2S,4R)-1-acetyl-4-[(4-chlorophenyl)amino]-2-methyl-3,4-dihydro-2H-quinolin-6-yl]phenyl]formamido)-3,6,9,12,15-pentaoxaoctadecan-18-oic acid was used, 31.60 mg of N-(5-[[2-([2-[(2-[[3-([3-[1-([4-[(2S,4R)-1-acetyl-4-[(4-chlorophenyl)amino]-2-methyl-3,4-dihydro-2H-quinolin-6-yl]phenyl]formamido)-3,6,9,12,15-pentaoxaoctadecan-18-amido]propyl](methyl)amino)propyl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide was obtained as white solid (9.48% yield). HRMS: mass calcd. For C₈₁H₁₀₆ClN₂₁O₁₆: 1663.7815, found: 1664.7825 [M+H]⁺.

Type 6 Reactions in the Synthesis of Transcription Modulator Molecules

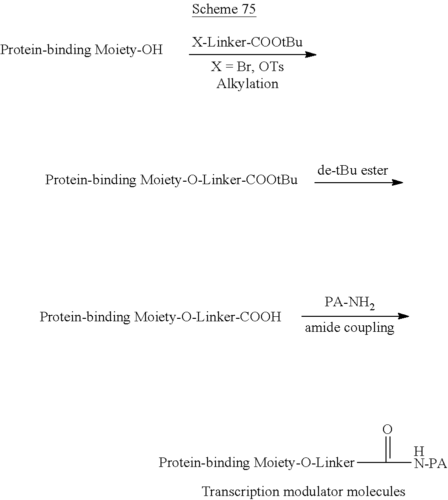

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 3)

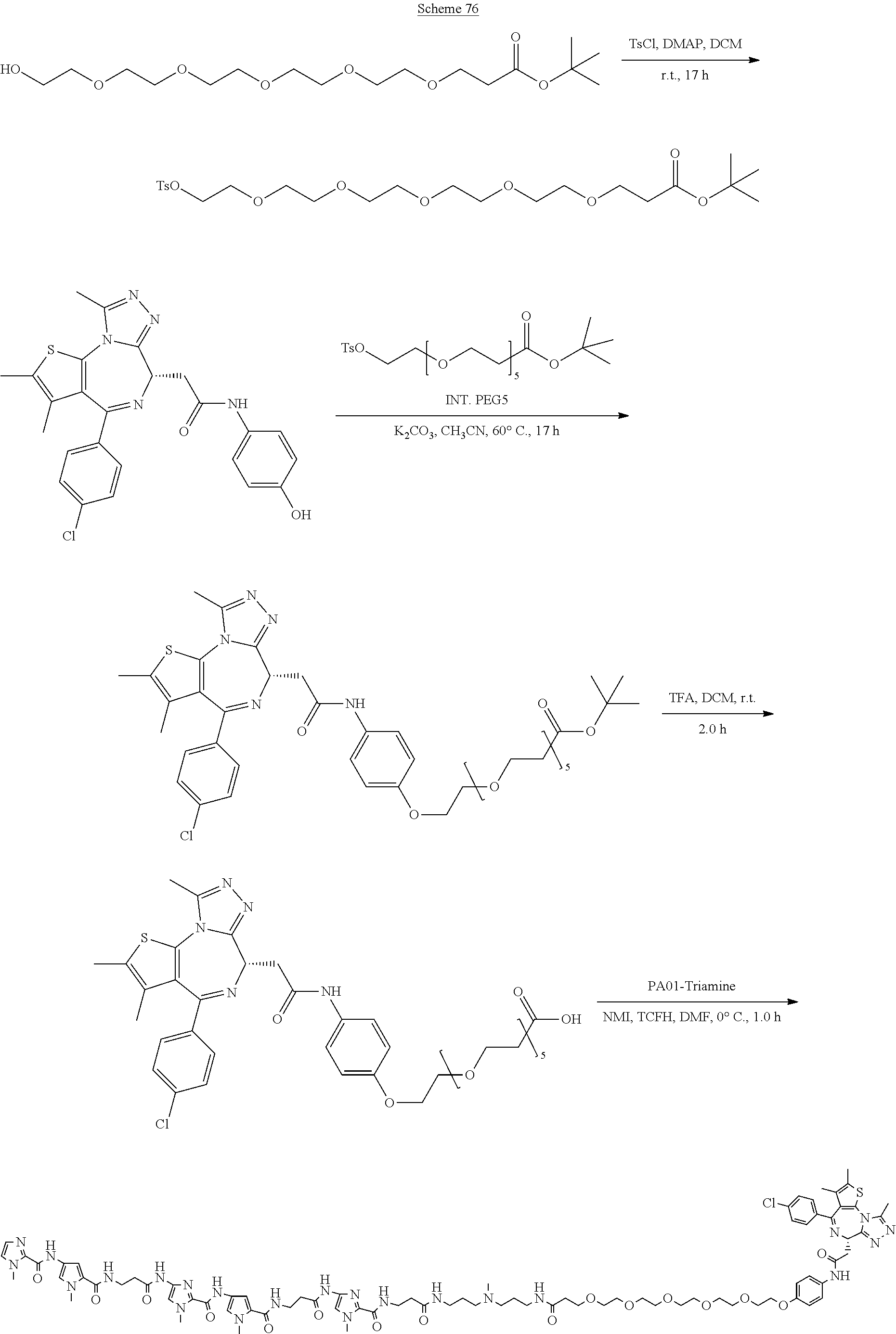

### Step 1: Synthesis of tert-butyl 1-[(4-methylbenzenesulfonyl)oxy]-3,6,9,12,15-oate

To a 50 mL round bottom flask was added tert-butyl 1-hydroxy-3,6,9,12,15-pentaoxaoctadecan-18-oate (500.00 mg, 1.36 mmol, 1.00 equiv) and DCM (10.00 mL). To this solution was added Et₃N (0.23 mL, 1.66 mmol, 1.21 equiv), DMAP (16.67 mg, 0.14 mmol, 0.10 equiv), finally was added TsCl (312.14 mg, 1.64 mmol, 1.20 equiv). The mixture was stirred at room temperature for 17 h. The resulting mixture was concentrated under reduced pressure. The residue was purified by silica gel column chromatography, elute with PE:EA=3:1. Tert-butyl 1-[(4-methylbenzenesulfonyl)oxy]-3,6,9,12,15-oate (700.00 mg, 95.00%) was obtained as colorless oil. LC/MS: mass calcd. For C₂₄H₄₀O₁₀S, Exact Mass: 520.23, found: 543.20 [M+Na]⁺.

### Step 2: Synthesis of tert-butyl (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oate

To a solution of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide (110.00 mg, 0.22 mmol, 1.00 equiv) in MeCN (10.00 mL) was added tert-butyl 1-[(4-methylbenzenesulfonyl)oxy]-3,6,9,12,15-pentaoxaoctadecan-18-oate (116.40 mg, 0.22 mmol, 1.00 equiv) and K₂CO₃ (61.80 mg, 0.45 mmol, 2.00 equiv). Then the reaction was stirred at 60 degrees C. for 17 h. The mixture was filtrated and concentrated. The residue was purified by reverse flash chromatography with the following conditions: column, C18 column; mobile phase, ACN in water (0.05% TFA), 5% to 50% gradient in 50 min; detector, UV 254 nm. The fractions were combined and concentrated to afford tert-butyl (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oate (180.00 mg, 95.70% yield). LC/MS: mass calcd. For C₄₂H₅₄ClN₅O₉S: 839.33 found: 840.55 [M+H]⁺.

### Step 3: Synthesis of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 200.00 mg of tert-butyl (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oate was used, 200.00 mg crude of (S)-1-(4-(2-(4-(4-chlorophenyl)-2,3,9-trimethyl-6H-thieno[3,2-f][1,2,4]triazolo [4,3-a][1,4]diazepin-6-yl)acetamido)phenoxy)-3,6,9,12,15-pentaoxaoctadecan-18-oic acid was obtained as yellow oil. LC/MS: mass calcd. For C₃₈H₄₆ClN₅O₉S: 783.27, found: 784.50 [M+H]⁺.

### Step 4: Synthesis of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

To a solution of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid (210.00 mg, 0.27 mmol, 1.00 equiv) in DMF (2.00 mL) was added NMI (219.83 mg, 2.68 mmol, 10.00 equiv) and TCFH (112.69 mg, 0.40 mmol, 1.50 equiv). Then N-[5-([2-[(2-[[2-([3-[(3-aminopropyl) (methyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide (256.25 mg, 0.27 mmol, 1.00 equiv) was added at 0 degree C. The reaction was stirred at 0 degrees C. for 1 h. Then the reaction mixture was purified by Prep-HPLC with the following conditions: Column: XBridge Prep Phenyl OBD Column, 19×150 mm 5 um 13 nm; Mobile Phase A: Water (10 MMOL/L NH₄HCO₃+0.1% NH₃.H₂O), Mobile Phase B: ACN; Flow rate: 25 mL/min; Gradient: 35 B to 42 B in 14 min; 254 nm; RT1: 12.92. The fraction were combined and lyophilized directly. (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (21.9 mg, 4.75% yield) was obtained as white solid. HRMS: mass calcd. For C₈₁H₁₀₄ClN₂₃O₁₆S: 1721.7441, found: 1722.7501 [M+H]⁺.

### (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18-oxo-3,6,9,12,15-pentaoxa-19,23-diazahexacosan-26-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (Compound 16)

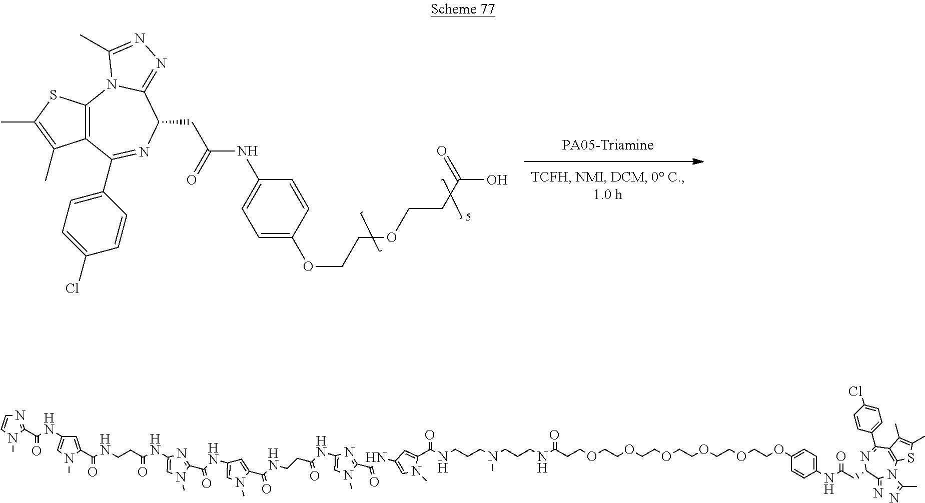

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 100.00 mg of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid was used, 26.90 mg of (S)—N-(5-((1-(4-(2-(4-(4-chlorophenyl)-2,3,9-trimethyl-6H-thieno[3,2-f][1,2,4] triazolo[4,3-a][1,4]diazepin-6-yl)acetamido)phenoxy)-23-methyl-18-oxo-3,6,9,12,15-pentaoxa-19,23-diazahexacosan-26-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide was obtained as white solid (11.72% yield). HRMS: mass calcd. For C₈₄H₁₀₅ClN₂₄O₁₆S: 1772.7550, found: 1773.7657 [M+H]⁺.

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-18,28-dioxo-23-(2,2,2-trifluoroethyl)-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 38)

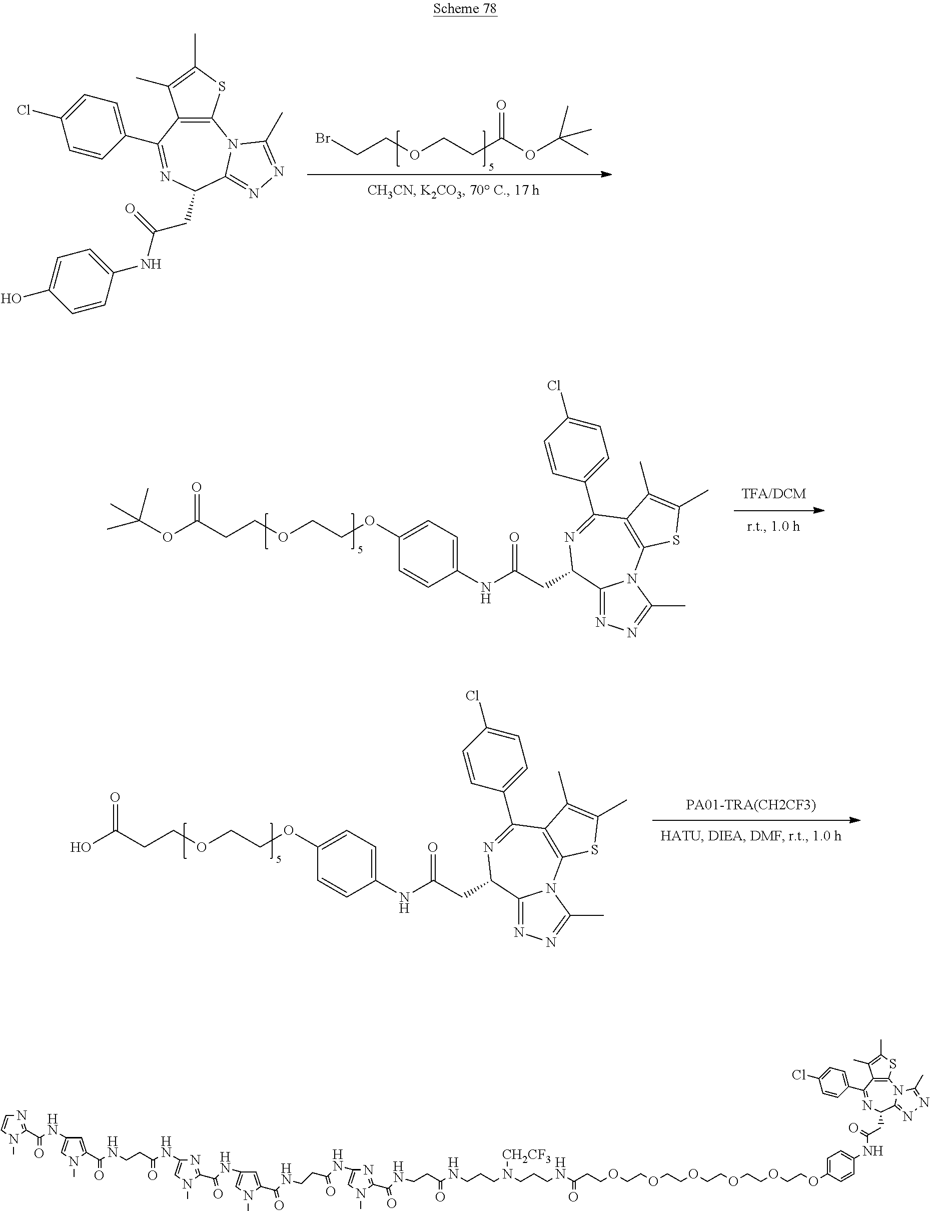

### Step 1: Synthesis of tert-butyl (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 100.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 190.00 mg crude of tert-butyl (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oate was obtained as yellow oil. LC/MS: mass calcd. For C₄₂H₅₄ClN₅O₉S: 839.33, found: 862.25[M+Na]⁺.

### Step 2: Synthesis of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 190.00 mg of tert-butyl (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oate was used, 190.00 mg of (S)-1-(4-(2-(4-(4-chlorophenyl)-2,3,9-trimethyl-6H-thieno[3,2-f][1,2,4]triazolo [4,3-a][1,4]diazepin-6-yl)acetamido)phenoxy)-3,6,9,12,15-pentaoxaoctadecan-18-oic acid was obtained as yellow oil. LC/MS: mass calcd. For C₃₈H₄₆ClN₅O₉S: 783.27, found: 784.14 [M+H]⁺.

### Step 3: Synthesis of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-18,28-dioxo-23-(2,2,2-trifluoroethyl)-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-2,24,34-trioxo-6,9,12,15,18,21-hexaoxa-3,25,29,33-tetraazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 70.00 mg of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid was used, 13.20 mg of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-18,28-dioxo-23-(2,2,2-trifluoroethyl)-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (7.83% yield). HRMS: mass calcd. For C₈₂H₁₀₃CF₃N₂₃O₁₆S: 1789.7314, found: 1790.7422 [M+H]⁺.

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19,23-dimethyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 53)

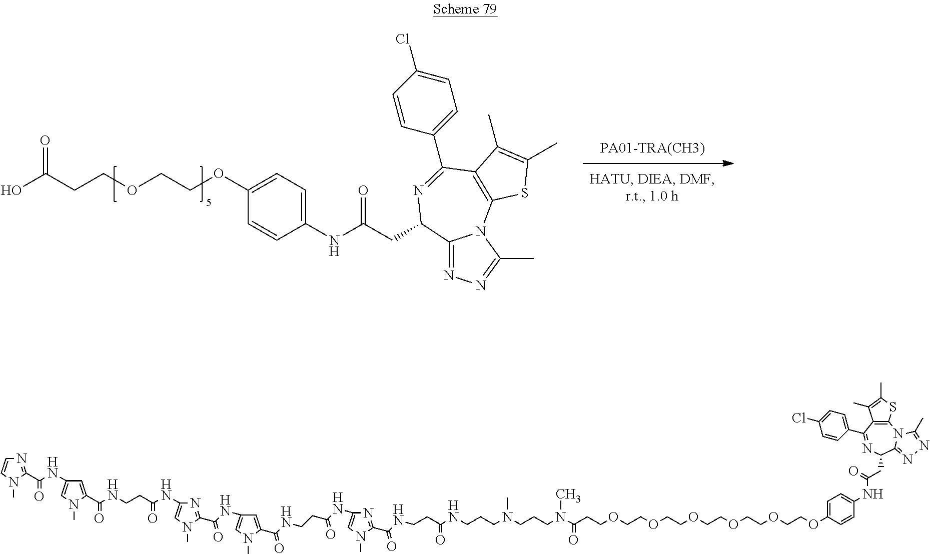

The procedure was the same as (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-2,24,34-trioxo-6,9,12,15,18,21-hexaoxa-3,25,29,33-tetraazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 50.00 mg of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid was used, 18.70 mg of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19,23-dimethyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (16.54% yield). HRMS: mass calcd. For C₈₂H₁₀₆ClN₂₃O₁₆S: 1735.76, found: 1736.7681 [M+H]⁺.

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19,23,27-trimethyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 54)

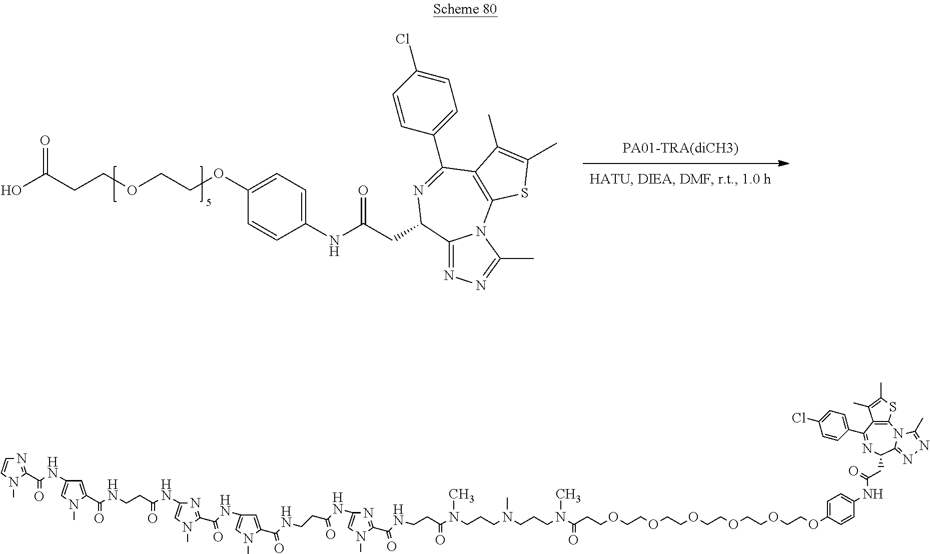

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 80.00 mg of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid was used, 17.60 mg of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19,23,27-trimethyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (9.81% yield). HRMS: mass calcd. For C₈₃H₁₀₈ClN₂₃O₁₆S: 1749.7754, found: 1750.7806 [M+H]⁺.

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-18,28-dioxo-3,6,9,12,15,23-hexaoxa-19,27-diazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 58)

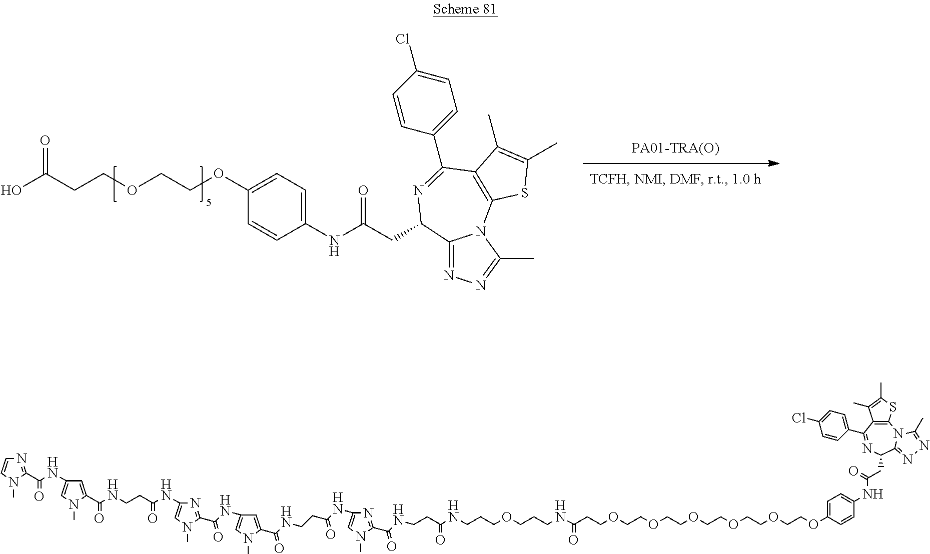

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 80.00 mg of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid was used, 21.30 mg of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-18,28-dioxo-3,6,9,12,15,23-hexaoxa-19,27-diazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (13.66% yield). HRMS: mass calcd. For C₈₀H₁₀₁ClN₂₂O₁₇S: 1708.71, found: 1709.7173 [M+H]⁺.

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-18,28-dioxo-3,6,9,12,15-pentaoxa-19,27-diazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 59)

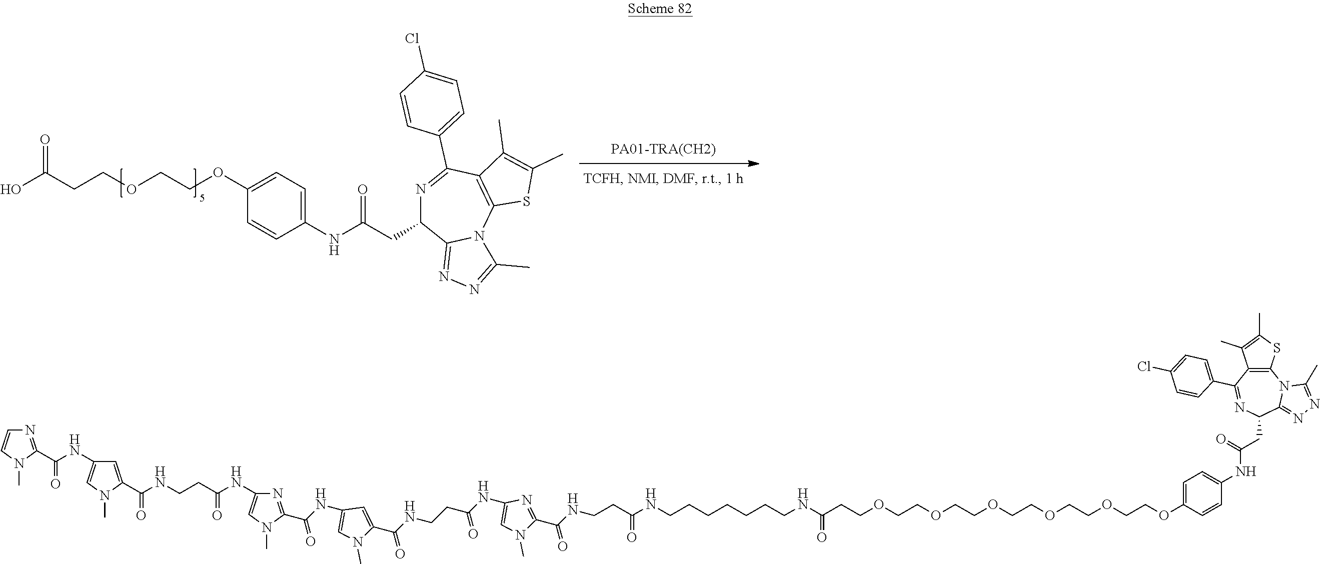

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 70.00 mg of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid was used, 17.10 mg of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-18,28-dioxo-3,6,9,12,15-pentaoxa-19,27-diazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (10.82% yield). HRMS: mass calcd. For C₈₁H₁₀₃ClN₂₂O₁₆S: 1706.7332, found: 1707.7401 [M+H]⁺.

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-4-palmitamido-1H-imidazole-2-carboxamido)-1H-pyrrole-2-carboxamido)propanamido)-1H-imidazole-2-carboxamide (Compound 66)

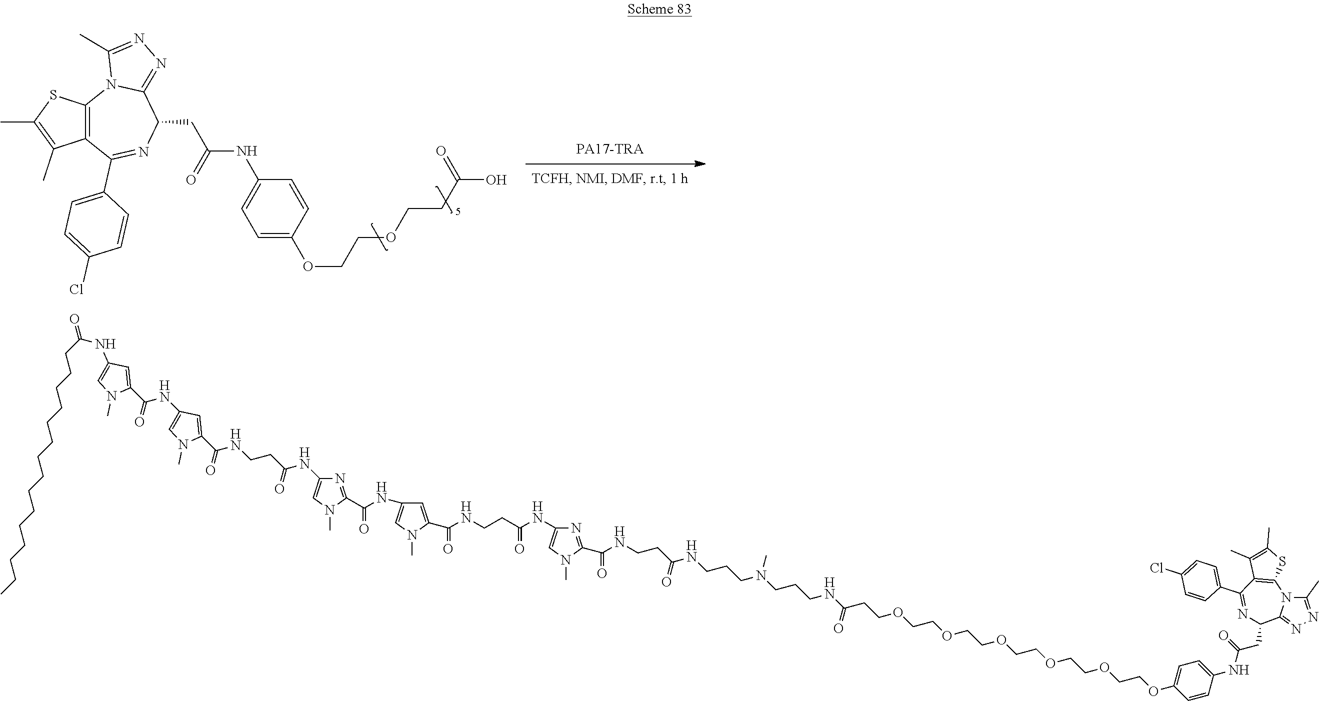

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 22.00 mg of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid was used, 5.40 mg of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-4-palmitamido-1H-imidazole-2-carboxamido)-1H-pyrrole-2-carboxamido)propanamido)-1H-imidazole-2-carboxamide was obtained as white solid (15.31% yield). HRMS: mass calcd. For C₉₇H₁₃₅ClN₂₄O₁₇S: 1974.9846, found: 1975.9928 [M+H]⁺.

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23,28-dioxo-3,6,9,12,15,18,21-heptaoxa-24,27-diazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 17)

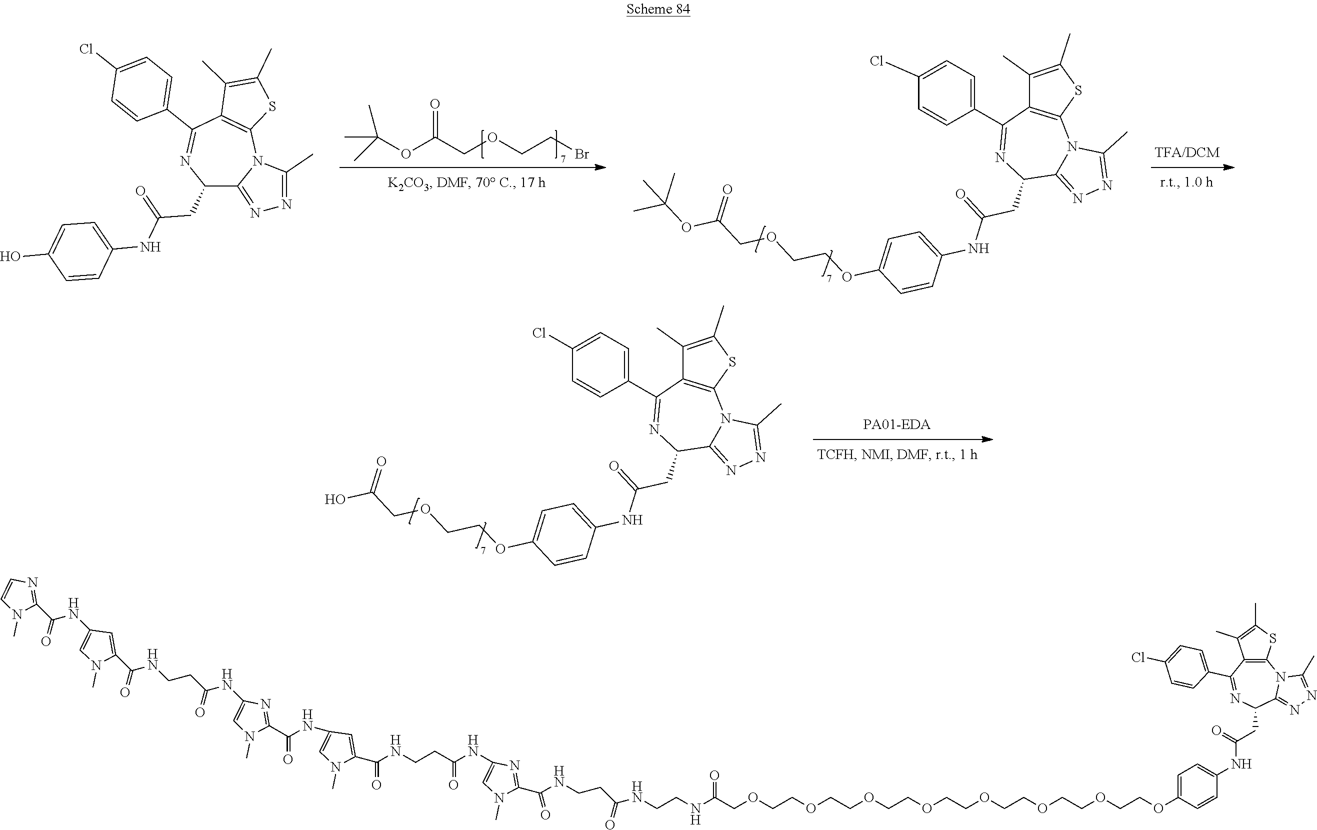

### Step 1: Synthesis of tert-butyl (S)-23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosanoate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 150.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 200.00 mg of tert-butyl (S)-23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosanoate was obtained as yellow oil (59.39% yield). LC/MS: mass calcd. For C₄₅H₆₀ClN₅O₁₁S: 913.37, found: 914.55 [M+H]⁺.

### Step 2: Synthesis of (S)-23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosanoic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 100.00 mg of tert-butyl (S)-23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosanoate was used, 100.00 mg crude of (S)-23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosanoic acid was obtained as yellow oil. LC/MS: mass calcd. For C₄₁H₅₂ClN₅O₁₁S: 857.31, found: 858.55 [M+H]⁺.

### Step 3: Synthesis of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23,28-dioxo-3,6,9,12,15,18,21-heptaoxa-24,27-diazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 100.00 mg of (S)-23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosanoic acid was used, 21.40 mg of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23,28-dioxo-3,6,9,12,15,18,21-heptaoxa-24,27-diazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (10.44% yield). HRMS: mass calcd. For C₇₉H₉₉ClN₂₂O₁₈S: 1710.6917, found: 1711.6923 [M+H]⁺.

### (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-methyl-23-oxo-3,6,9,12,15,18,21-heptaoxa-24,28-diazahentriacontan-31-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (Compound 57)

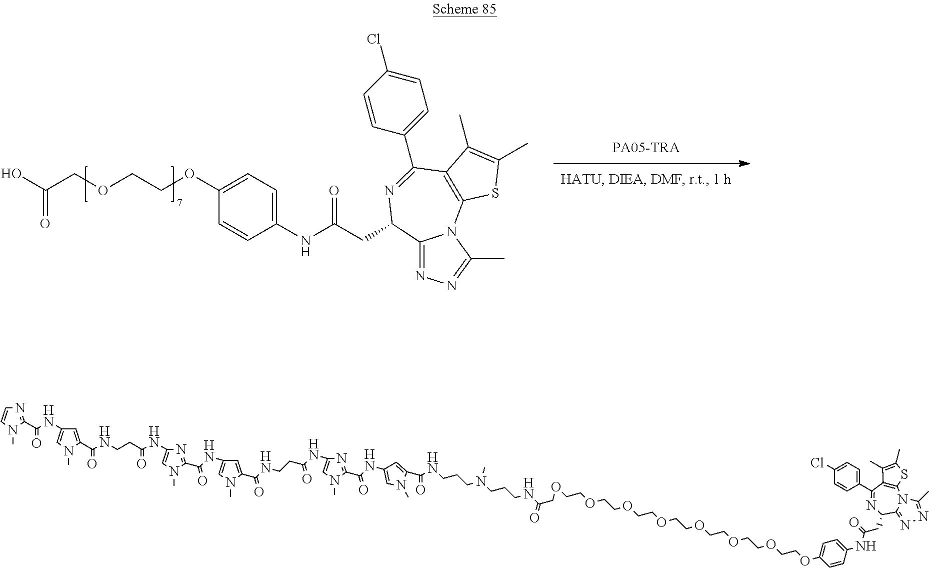

The procedure was the same as (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-2,24,34-trioxo-6,9,12,15,18,21-hexaoxa-3,25,29,33-tetraazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 80.00 mg of (S)-23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosanoic acid was used, 14.90 mg of (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-28-methyl-23-oxo-3,6,9,12,15,18,21-heptaoxa-24,28-diazahentriacontan-31-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide was obtained as white solid (8.62% yield). HRMS: mass calcd. For C₈₇H₁₁₁ClN₂₄O₁₈S: 1846.7917, found: 1847.7949 [M+H]⁺.

### (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-29-methyl-24-oxo-3,6,9,12,15,18,21-heptaoxa-25,29-diazadotriacontan-32-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (Compound 56)

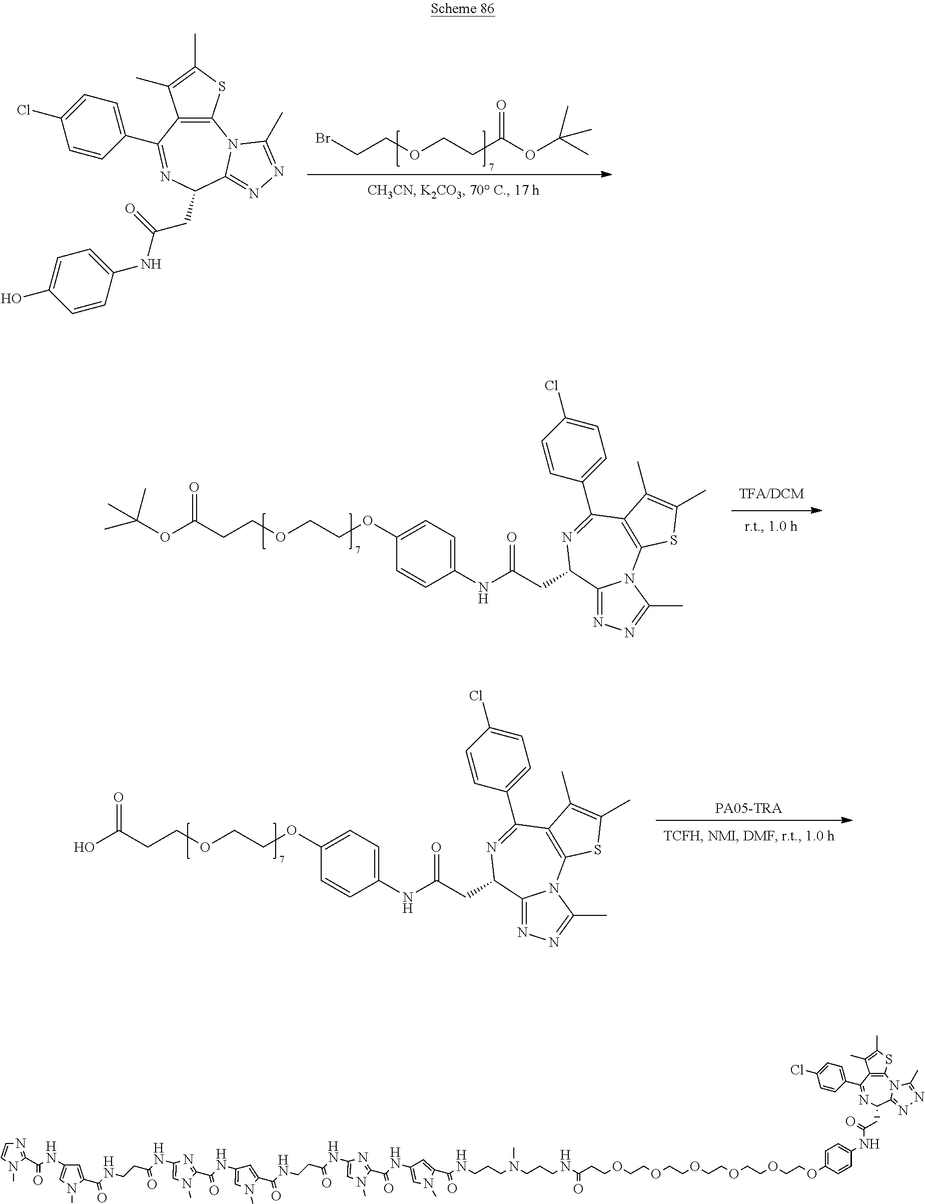

### Step 1: Synthesis of tert-butyl (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatetracosan-24-oate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 100.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 113.00 mg of tert-butyl (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatetracosan-24-oate was obtained as yellow oil (57.48% yield). LC/MS: mass calcd. For C₃₄H₃₈ClN₅O₅S: 927.38, found: 465.25 [M/2+H]⁺.

### Step 2: Synthesis of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatetracosan-24-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic. 113.00 mg of tert-butyl (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatetracosan-24-oate was used, 158.00 mg crude of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatetracosan-24-oic acid was obtained as yellow oil. LCMS: mass calcd. For C₄₂H₅₄ClN₅O₁₁S: 871.32, found: 894.60 [M+Na]⁺.

### Step 3: Synthesis of (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-29-methyl-24-oxo-3,6,9,12,15,18,21-heptaoxa-25,29-diazadotriacontan-32-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 138.00 mg of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatetracosan-24-oic acid was used, 16.50 mg of (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-29-methyl-24-oxo-3,6,9,12,15,18,21-heptaoxa-25,29-diazadotriacontan-32-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide was obtained as white solid (7.92% yield). HRMS: mass calcd. For C₈₈H₁₁₃ClN₂₄O₁₈S: 1860.8074, found: 1861.8131 [M+H]⁺.

### (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-26-methyl-21-oxo-3,6,9,12,15,18-hexaoxa-22,26-diazanonacosan-29-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (Compound 55)

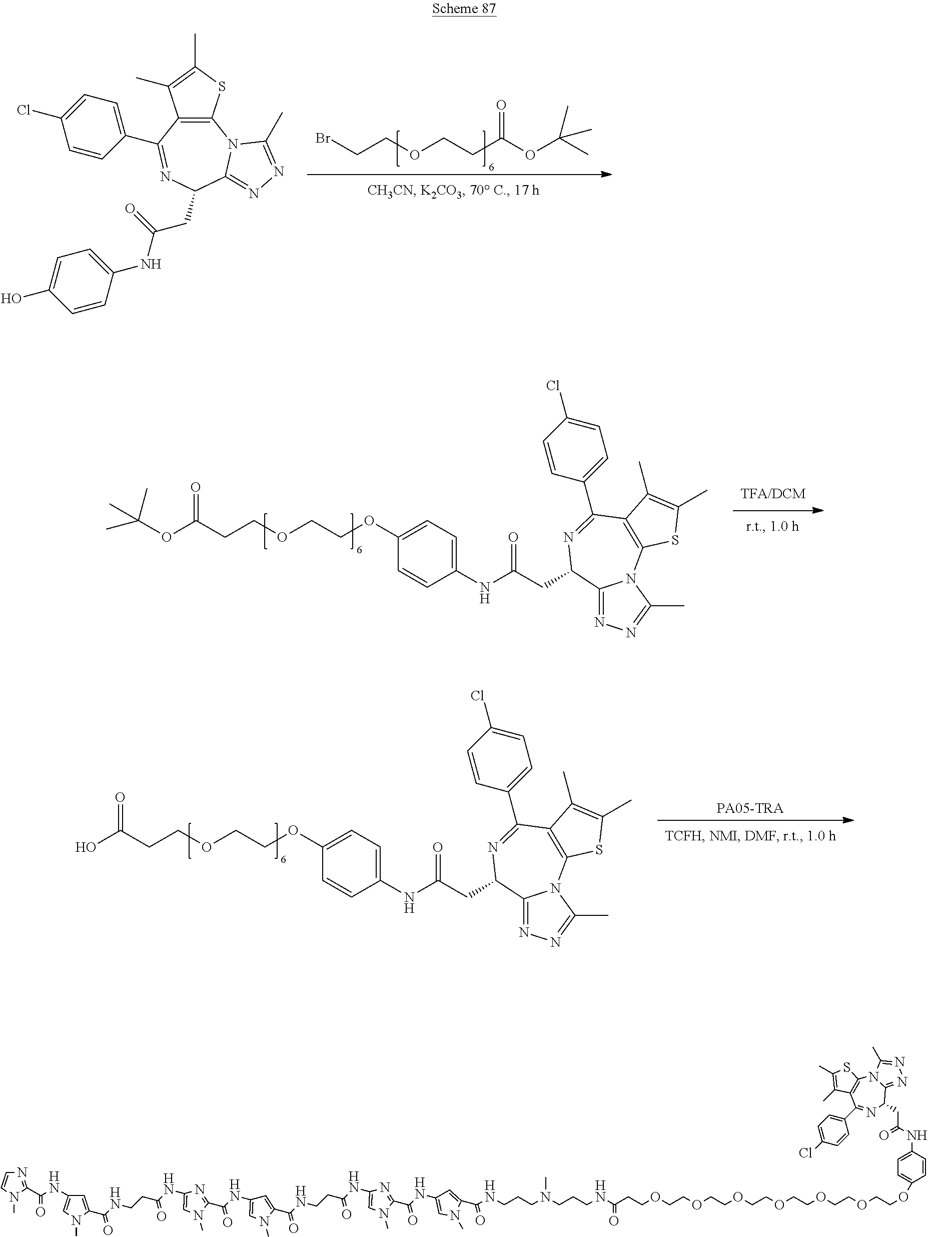

### Step 1: Synthesis of tert-butyl (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18-hexaoxahenicosan-21-oate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 50.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 80.00 mg of tert-butyl (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18-hexaoxahenicosan-21-oate was obtained as yellow oil (85.13% yield). LC/MS: mass calcd. For C₄₄H₅₈ClN₅O₁₀S: 883.36, found: 884.60 [M+H]⁺.

### Step 2: Synthesis of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18-hexaoxahenicosan-21-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 80.00 mg of tert-butyl (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18-hexaoxahenicosan-21-oate was used, 80.00 mg crude of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18-hexaoxahenicosan-21-oic acid was obtained as yellow oil. LC/MS: mass calcd. For C₄₀H₅₀ClN₅₀O₁₀S: 827.30, found: 828.50 [M+H]⁺.

### Step 3: Synthesis of (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-26-methyl-21-oxo-3,6,9,12,15,18-hexaoxa-22,26-diazanonacosan-29-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 100.00 mg crude of (S)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18-hexaoxahenicosan-21-oic acid was used, 20.30 mg of (S)—N-(5-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-26-methyl-21-oxo-3,6,9,12,15,18-hexaoxa-22,26-diazanonacosan-29-yl)carbamoyl)-1-methyl-1H-pyrrol-3-yl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide was obtained as white solid (8.91% yield). HRMS: mass calcd. For C₈₆H₁₀₉ClN₂₄O₁₇S: 1816.7812, found: 1817.7858 [M+H]⁺.

### (S)-4-(3-(4-(4-(23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosanamido)-1-methyl-1H-imidazole-2-carboxamido)-1-methyl-1H-pyrrole-2-carboxamido)propanamido)-1-methyl-N-(1-methyl-5-((3-((1-methyl-2-((3-oxo-3-(propylamino)propyl)carbamoyl)-1H-imidazol-4-yl)amino)-3-oxopropyl)carbamoyl)-1H-pyrrol-3-yl)-1H-imidazole-2-carboxamide (Compound 49)

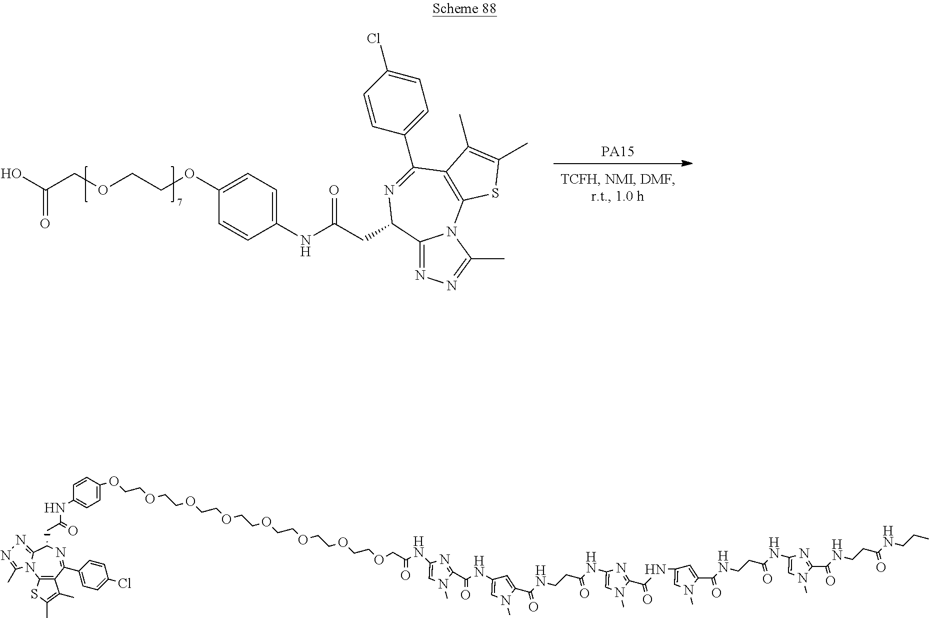

### Step 1: Synthesis of (S)-4-(3-(4-(4-(23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosanamido)-1-methyl-1H-imidazole-2-carboxamido)-1-methyl-1H-pyrrole-2-carboxamido)propanamido)-1-methyl-N-(1-methyl-5-((3-((1-methyl-2-((3-oxo-3-(propylamino)propyl)carbamoyl)-1H-imidazol-4-yl)amino)-3-oxopropyl)carbamoyl)-1H-pyrrol-3-yl)-1H-imidazole-2-carboxamide (Compound 49)

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 80.00 mg of (S)-23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosanoic acid was used, 5.20 mg of (S)-4-(3-(4-(4-(23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosanamido)-1-methyl-1H-imidazole-2-carboxamido)-1-methyl-1H-pyrrole-2-carboxamido)propanamido)-1-methyl-N-(1-methyl-5-((3-((1-methyl-2-((3-oxo-3-(propylamino)propyl)carbamoyl)-1H-imidazol-4-yl)amino)-3-oxopropyl)carbamoyl)-1H-pyrrol-3-yl)-1H-imidazole-2-carboxamide was obtained as white solid (3.12% yield). HRMS: mass calcd. For C₈₀H₁₀₁ClN₂₂O₁₈S: 1724.7073, found: 1725.7118 [M+H]⁺.

### (S)-4-(3-(4-(4-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosanamido)-1-methyl-1H-imidazole-2-carboxamido)-1-methyl-1H-pyrrole-2-carboxamido)propanamido)-1-methyl-N-(1-methyl-5-((3-((1-methyl-2-((3-oxo-3-(propylamino)propyl)carbamoyl)-1H-imidazol-4-yl)amino)-3-oxopropyl)carbamoyl)-1H-pyrrol-3-yl)-1H-imidazole-2-carboxamide (Compound 50)

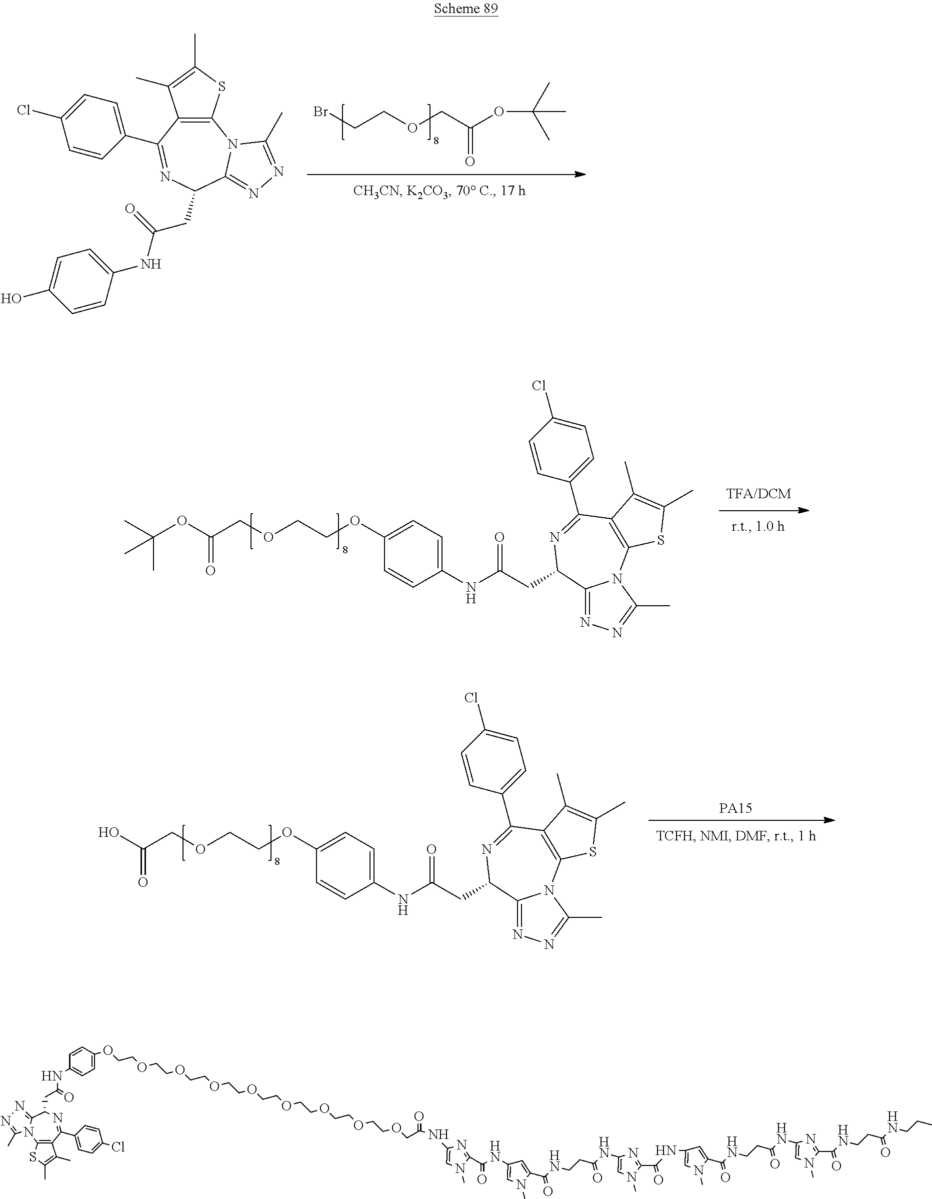

### Step 1: Synthesis of tert-butyl (S)-26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosanoate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 100.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 140.00 mg of tert-butyl (S)-26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosanoate was obtained as yellow oil (68.50% yield). LC/MS: mass calcd. For C₄₇H₆₄ClN₅O₁₂S: 957.40, found: 981.60 [M+Na]⁺.

### Step 2: Synthesis of (S)-26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosanoic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 140.00 mg of tert-butyl (S)-26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosanoate was used, 140.00 mg of (S)-26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosanoic acid was obtained as yellow oil. LC/MS: mass calcd. For C₄₃H₅₆ClN₅O₁₂S: 901.33, found: 924.60 [M+Na]⁺.

### Step 3: Synthesis of (S)-4-(3-(4-(4-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosanamido)-1-methyl-1H-imidazole-2-carboxamido)-1-methyl-1H-pyrrole-2-carboxamido)propanamido)-1-methyl-N-(1-methyl-5-((3-((1-methyl-2-((3-oxo-3-(propylamino)propyl)carbamoyl)-1H-imidazol-4-yl)amino)-3-oxopropyl)carbamoyl)-1H-pyrrol-3-yl)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 60.00 mg of (S)-26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosanoic acid was used, 2.30 mg of (S)-4-(3-(4-(4-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosanamido)-1-methyl-1H-imidazole-2-carboxamido)-1-methyl-1H-pyrrole-2-carboxamido)propanamido)-1-methyl-N-(1-methyl-5-((3-((1-methyl-2-((3-oxo-3-(propylamino)propyl)carbamoyl)-1H-imidazol-4-yl)amino)-3-oxopropyl)carbamoyl)-1H-pyrrol-3-yl)-1H-imidazole-2-carboxamide was obtained as white solid (1.94% yield). HRMS: mass calcd. For C₈₂H₁₀₅ClN₂₂O₁₉S: 1768.7336, found: 1769.7437 [M+H]⁺.

### (S)-4-(3-(4-(4-(1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-oxo-3,6,9,12,15,18,21-heptaoxa-24-azaheptacosan-27-amido)-1-methyl-1H-imidazole-2-carboxamido)-1-methyl-1H-pyrrole-2-carboxamido)propanamido)-1-methyl-N-(1-methyl-5-((3-((1-methyl-2-(propylcarbamoyl)-1H-imidazol-4-yl)amino)-3-oxopropyl)carbamoyl)-1H-pyrrol-3-yl)-1H-imidazole-2-carboxamide (Compound 51)

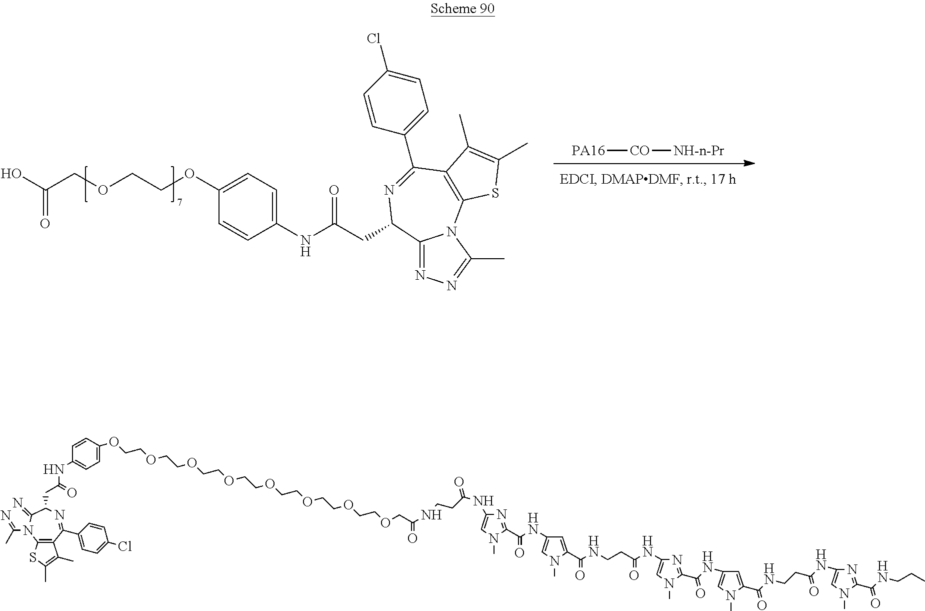

The procedure was the same as N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide. 78.00 mg of (S)-23-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21-heptaoxatricosanoic acid was used, 18.80 mg of (S)-4-(3-(4-(4-(1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-oxo-3,6,9,12,15,18,21-heptaoxa-24-azaheptacosan-27-amido)-1-methyl-1H-imidazole-2-carboxamido)-1-methyl-1H-pyrrole-2-carboxamido)propanamido)-1-methyl-N-(1-methyl-5-((3-((1-methyl-2-(propylcarbamoyl)-1H-imidazol-4-yl)amino)-3-oxopropyl)carbamoyl)-1H-pyrrol-3-yl)-1H-imidazole-2-carboxamide was obtained as white solid (11.85% yield). HRMS: mass calcd. For C₈₀H₁₀₁ClN₂₂O₁₈S: 1724.7073, found: 1725.7189 [M+H]⁺.

### (S)-4-(3-(4-(4-(1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-26-oxo-3,6,9,12,15,18,21,24-octaoxa-27-azatriacontan-30-amido)-1-methyl-1H-imidazole-2-carboxamido)-1-methyl-1H-pyrrole-2-carboxamido)propanamido)-1-methyl-N-(1-methyl-5-((3-((1-methyl-2-(propylcarbamoyl)-1H-imidazol-4-yl)amino)-3-oxopropyl)carbamoyl)-1H-pyrrol-3-yl)-1H-imidazole-2-carboxamide (Compound 52)

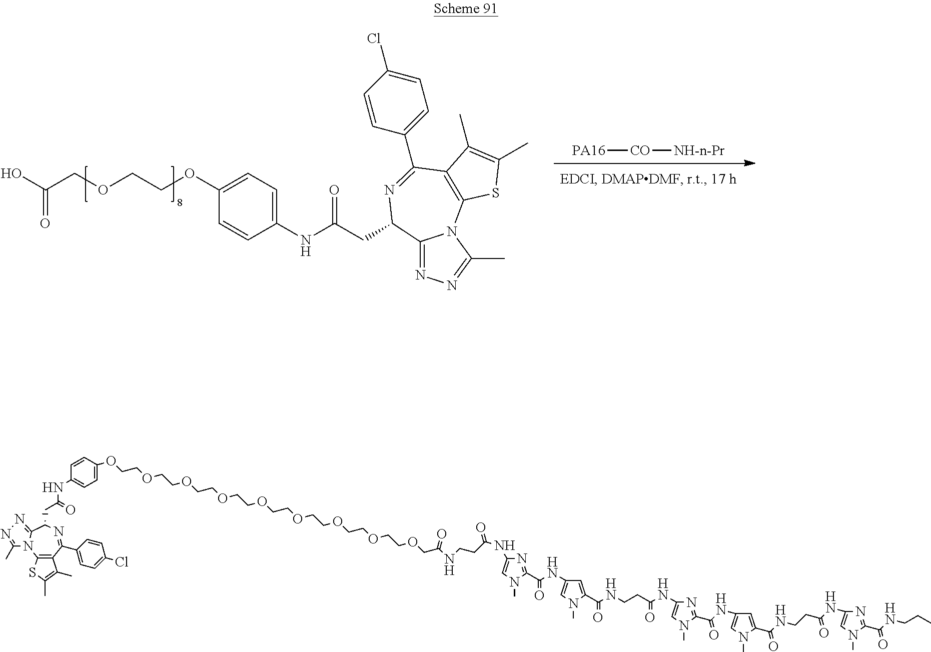

The procedure was the same as N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide. 71.00 mg of (S)-26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosanoic acid was used, 17.10 mg of (S)-4-(3-(4-(4-(1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-26-oxo-3,6,9,12,15,18,21,24-octaoxa-27-azatriacontan-30-amido)-1-methyl-1H-imidazole-2-carboxamido)-1-methyl-1H-pyrrole-2-carboxamido)propanamido)-1-methyl-N-(1-methyl-5-((3-((1-methyl-2-(propylcarbamoyl)-1H-imidazol-4-yl)amino)-3-oxopropyl)carbamoyl)-1H-pyrrol-3-yl)-1H-imidazole-2-carboxamide was obtained as white solid (11.88% yield). HRMS: mass calcd. For C₈₂H₁₀₅ClN₂₂O₁₉S: 1768.7336, found: 1769.7389 [M+H]⁺.

### (S)—N-(5-((3-((2-((1-(4-(2-(6-(4-chlorophenyl)-8-methoxy-1-methyl-4H-benzo[f][1,2,4]triazolo[4,3-a][1,4]diazepin-4-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 6)

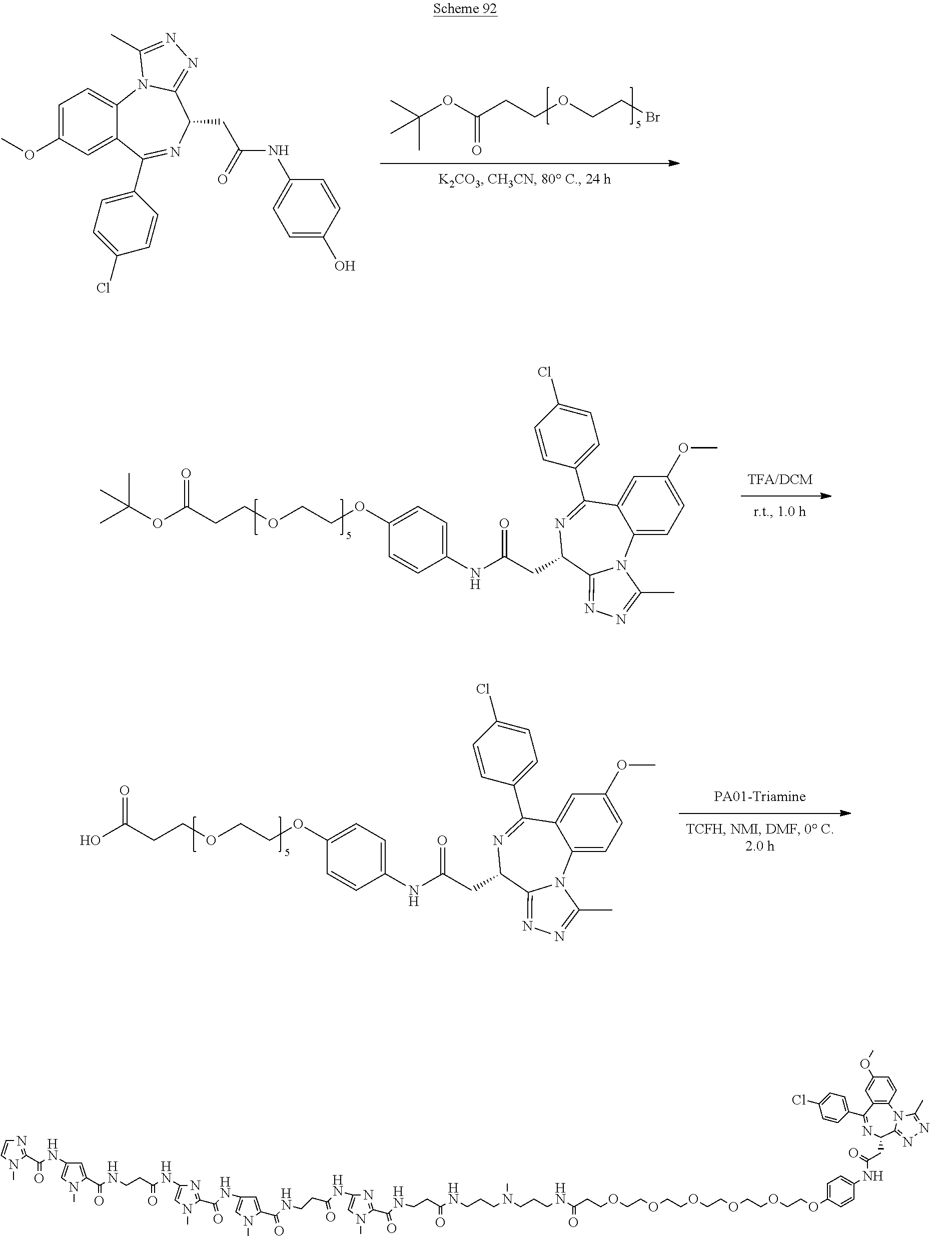

### Step 1: Synthesis of tert-butyl (S)-1-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15-pentaoxaoctadecan-18-oate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 120.00 mg of (S)-2-(6-(4-chlorophenyl)-8-methoxy-1-methyl-4H-benzo[f][1,2,4]triazolo[4,3-a][1,4]diazepin-4-yl)-N-(4-hydroxyphenyl)acetamide was used, 60.00 mg of tert-butyl (S)-1-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15-pentaoxaoctadecan-18-oate was obtained as yellow oil (25.96% yield). LC/MS: mass calcd. For C₄₃H₅₄ClN₅O₁₀: 835.35, found: 858.25 [M+Na]⁺.

### Step 2: Synthesis of (S)-1-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 120.00 mg of tert-butyl (S)-1-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15-pentaoxaoctadecan-18-oate was used, 120.00 mg crude of (S)-1-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15-pentaoxaoctadecan-18-oic acid was obtained as yellow oil. LC/MS: mass calcd. For C₃₉H₄₆ClN₅O₁₀: 779.29, found: 780.45 [M+H]⁺.

### Step 3: Synthesis of (S)—N-(5-((3-((2-((1-(4-(2-(6-(4-chlorophenyl)-8-methoxy-1-methyl-4H-benzo[f][1,2,4]triazolo[4,3-a][1,4]diazepin-4-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 120.00 mg of (S)-1-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15-pentaoxaoctadecan-18-oic acid was used, 29.60 mg of (S)—N-(5-((3-((2-((1-(4-(2-(6-(4-chlorophenyl)-8-methoxy-1-methyl-4H-benzo[f][1,2,4]triazolo[4,3-a][1,4]diazepin-4-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (11.04% yield). HRMS: mass calcd. For C₈₂H₁₀₄ClN₂₃O₁₇: 1717.7669, found: 1718.7736 [M+H]⁺.

### (S)—N-(5-((3-((2-((1-(4-(2-(6-(4-chlorophenyl)-1-methyl-4H-benzo[c]isoxazolo[4,5-e]azepin-4-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 7)

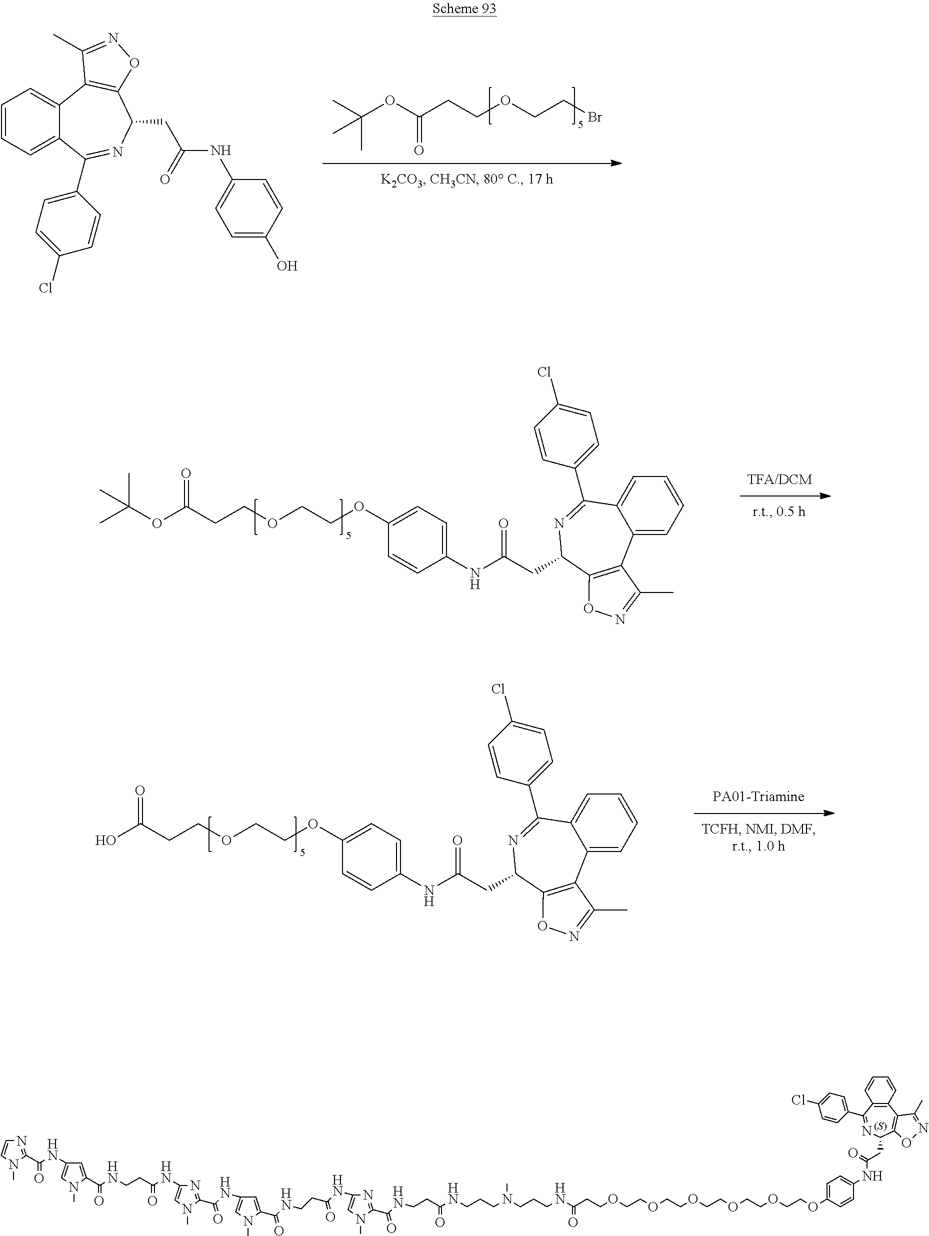

### Step 1: Synthesis of tert-butyl (S)-1-(4-(2-(6-(4-chlorophenyl)-1-methyl-4H-benzo[c]isoxazolo[4,5-e]azepin-4-yl)acetamido)phenoxy)-3,6,9,12,15-pentaoxaoctadecan-18-oate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate, but the reaction temperature was 80 degree C. 100.00 mg of (S)-2-(6-(4-chlorophenyl)-1-methyl-4H-benzo[c]isoxazolo[4,5-e]azepin-4-yl)-N-(4-hydroxyphenyl)acetamide was used, 70.00 mg of tert-butyl (S)-1-(4-(2-(6-(4-chlorophenyl)-1-methyl-4H-benzo[c]isoxazolo[4,5-e]azepin-4-yl)acetamido)phenoxy)-3,6,9,12,15-pentaoxaoctadecan-18-oate was obtained as yellow solid (38.05% yield). LC/MS: mass calcd. For C₄₃H₅₂ClN₃O₁₀: 805.33, found: 806.50 [M+H]⁺.

### Step 2: Synthesis of (S)-1-(4-(2-(6-(4-chlorophenyl)-1-methyl-4H-benzo[c]isoxazolo[4,5-e]azepin-4-yl)acetamido)phenoxy)-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 70.00 mg of tert-butyl (S)-1-(4-(2-(6-(4-chlorophenyl)-1-methyl-4H-benzo[c]isoxazolo[4,5-e]azepin-4-yl)acetamido)phenoxy)-3,6,9,12,15-pentaoxaoctadecan-18-oate was used, 70.00 mg crude of (S)-1-(4-(2-(6-(4-chlorophenyl)-1-methyl-4H-benzo[c]isoxazolo[4,5-e]azepin-4-yl)acetamido)phenoxy)-3,6,9,12,15-pentaoxaoctadecan-18-oic acid was obtained as yellow oil. LC/MS: mass calcd. For C₃₉H₄₄ClN₃O₁₀: 749.27, found: 750.40 [M+H]⁺.

### Step 3: Synthesis of (S)—N-(5-((3-((2-((1-(4-(2-(6-(4-chlorophenyl)-1-methyl-4H-benzo[c]isoxazolo[4,5-e]azepin-4-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 60.00 mg of (S)-1-(4-(2-(6-(4-chlorophenyl)-1-methyl-4H-benzo[c]isoxazolo[4,5-e]azepin-4-yl)acetamido)phenoxy)-3,6,9,12,15-pentaoxaoctadecan-18-oic acid was used, 26.80 mg of (S)—N-(5-((3-((2-((1-(4-(2-(6-(4-chlorophenyl)-1-methyl-4H-benzo[c]isoxazolo[4,5-e]azepin-4-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (19.61% yield). HRMS: mass calcd. For C₈₂H₁₀₂ClN₂₁O₁₇: 1687.7451, found: 1688.7509 [M+H]⁺.

### (R)—N-(5-((3-((2-((1-(4-(2-(6-(4-chlorophenyl)-8-methoxy-1-methyl-4H-benzo[f][1,2,4]triazolo[4,3-a][1,4]diazepin-4-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 11)

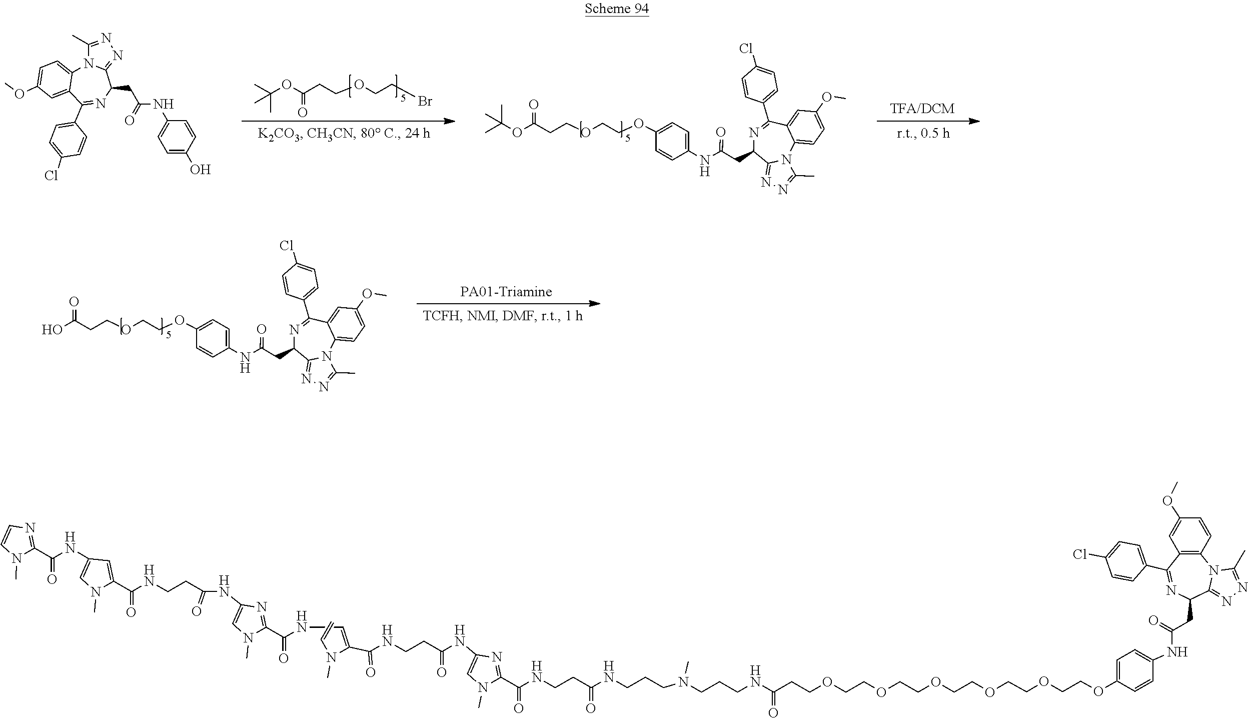

### Step 1: Synthesis of tert-butyl (R)-1-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15-pentaoxaoctadecan-18-oate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate, but the reaction temperature was 80 degree C., and the reaction time was 24 h. 120.00 mg of (R)-2-(6-(4-chlorophenyl)-8-methoxy-1-methyl-4H-benzo[f][1,2,4]triazolo[4,3-a][1,4]diazepin-4-yl)-N-(4-hydroxyphenyl)acetamide was used, 86.00 mg of tert-butyl (R)-1-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15-pentaoxaoctadecan-18-oate was obtained as yellow oil (37.00% yield). LC/MS: mass calcd. For C₄₃H₅₄ClN₅O₁₀: 835.36, found: 836.50 [M+H]⁺.

### Step 2: Synthesis of (R)-1-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 120.00 mg of tert-butyl (R)-1-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15-pentaoxaoctadecan-18-oate was used, 120.00 mg crude of (R)-1-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15-pentaoxaoctadecan-18-oic acid was obtained as yellow oil. LC/MS: mass calcd. For C₃₉H₄₆ClN₅O₁₀: 779.29, found: 780.20 [M+H]⁺.

### Step 3: Synthesis of (R)—N-(5-((3-((2-((1-(4-(2-(6-(4-chlorophenyl)-8-methoxy-1-methyl-4H-benzo[f][1,2,4]triazolo[4,3-a][1,4]diazepin-4-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 80.00 mg of (R)-1-(4-(2-(6-(4-chlorophenyl)-8-methoxy-1-methyl-4H-benzo[f][1,2,4]triazolo[4,3-a][1,4]diazepin-4-yl)acetamido)phenoxy)-3,6,9,12,15-pentaoxaoctadecan-18-oic acid was used, 23.10 mg of (R)—N-(5-((3-((2-((1-(4-(2-(6-(4-chlorophenyl)-8-methoxy-1-methyl-4H-benzo [f][1,2,4]triazolo[4,3-a][1,4]diazepin-4-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (12.79% yield). HRMS: mass calcd. For C₈₂H₁₀₄ClN₂₃O₁₇: 1717.7669, found: 1718.7760 [M+H]⁺.

### N-[5-([2-[(2-[[2-([3-[(3-[1-[3-(5,7-dimethoxy-4-oxo-3H-quinazolin-2-yl)-5-methylphenoxy]-3,6,9,12,15,18-hexaoxahenicosan-21-amido]propyl)(methyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide (Compound 12)

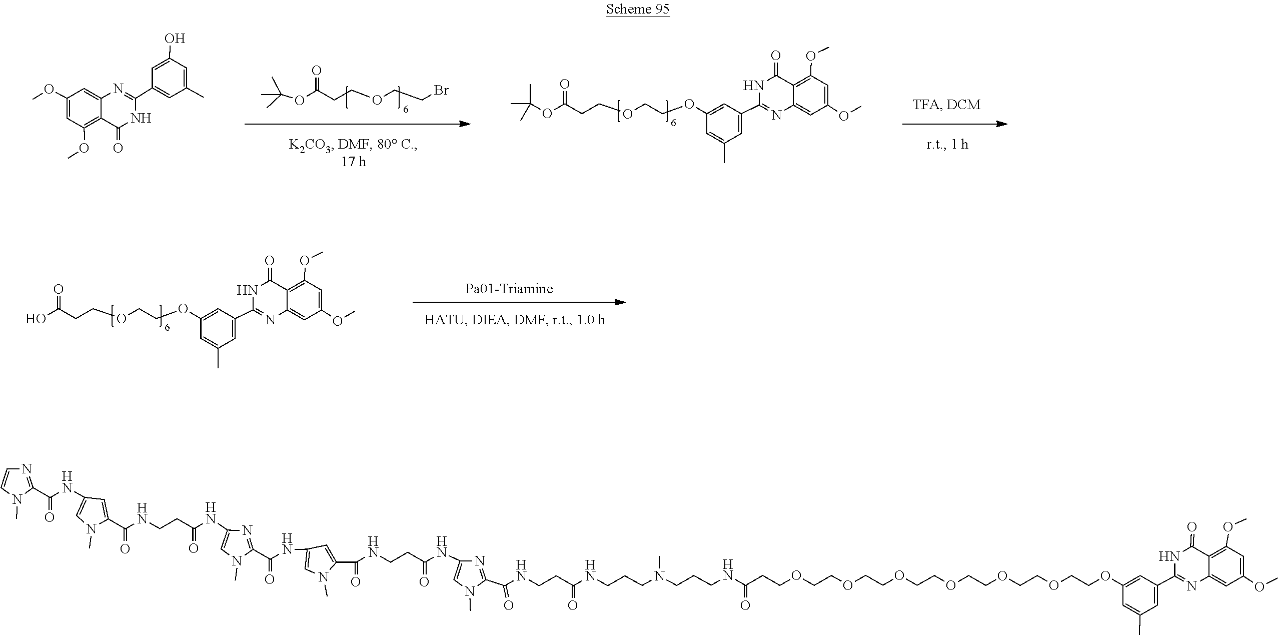

### Step 1: Synthesis of tert-butyl 1-[3-(5,7-dimethoxy-4-oxo-3H-quinazolin-2-yl)-5-methylphenoxy]-3,6,9,12,15,18-hexaoxahenicosan-21-oate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate, but the reaction temperature was 80 degree C. 200.00 mg of 2-(3-hydroxy-5-methylphenyl)-5,7-dimethoxy-3H-quinazolin-4-one was used, 280.00 mg of tert-butyl 1-[3-(5,7-dimethoxy-4-oxo-3H-quinazolin-2-yl)-5-methylphenoxy]-3,6,9,12,15,18-hexaoxahenicosan-21-oate was obtained as yellow solid (55.55% yield). LC/MS: mass calcd. For C₃₆H₅₂N₂O₁₂: 704.35, found: 705.50 [M+H]⁺.

### Step 2: Synthesis of 1-[3-(5,7-dimethoxy-4-oxo-3H-quinazolin-2-yl)-5-methylphenoxy]-3,6,9,12,15,18-hexaoxahenicosan-21-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 130.00 mg of tert-butyl 1-[3-(5,7-dimethoxy-4-oxo-3H-quinazolin-2-yl)-5-methylphenoxy]-3,6,9,12,15,18-hexaoxahenicosan-21-oate was used, 130.00 mg crude of 1-[3-(5,7-dimethoxy-4-oxo-3H-quinazolin-2-yl)-5-methylphenoxy]-3,6,9,12,15,18-hexaoxahenicosan-21-oic acid was obtained as yellow oil (87.14% yield). LC/MS: mass calcd. For C₃₂H₄₄N₂O₁₂: 648.29, found: 649.45 [M+H]⁺.

### Step 3: Synthesis of N-[5-([2-[(2-[[2-([3-[(3-[1-[3-(5,7-dimethoxy-4-oxo-3H-quinazolin-2-yl)-5-methylphenoxy]-3,6,9,12,15,18-hexaoxahenicosan-21-amido]propyl)(methyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-2,24,34-trioxo-6,9,12,15,18,21-hexaoxa-3,25,29,33-tetraazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 130.00 mg of 1-[3-(5,7-dimethoxy-4-oxo-3H-quinazolin-2-yl)-5-methylphenoxy]-3,6,9,12,15,18-hexaoxahenicosan-21-oic acid was used, 20.00 mg of N-[5-([2-[(2-[[2-([3-[(3-[1-[3-(5,7-dimethoxy-4-oxo-3H-quinazolin-2-yl)-5-methylphenoxy]-3,6,9,12,15,18-hexaoxahenicosan-21-amido]propyl)(methyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as white solid (6.19% yield). HRMS: mass calcd. For C₇₅H₁₀₂N₂₀O₁₉: 1586.7630, found: 1587.7682 [M+H]⁺.

### N-[5-[(2-[[2-([2-[(3-[[3-(1-[[7-(3,5-dimethyl-1,2-oxazol-4-yl)-1-[(2R)-1-methoxypropan-2-yl]-2-(oxan-4-yl)imidazo[4,5-c]quinolin-8-yl]oxy]-3,6,9,12,15,18-hexaoxahenicosan-21-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide (Compound 18)

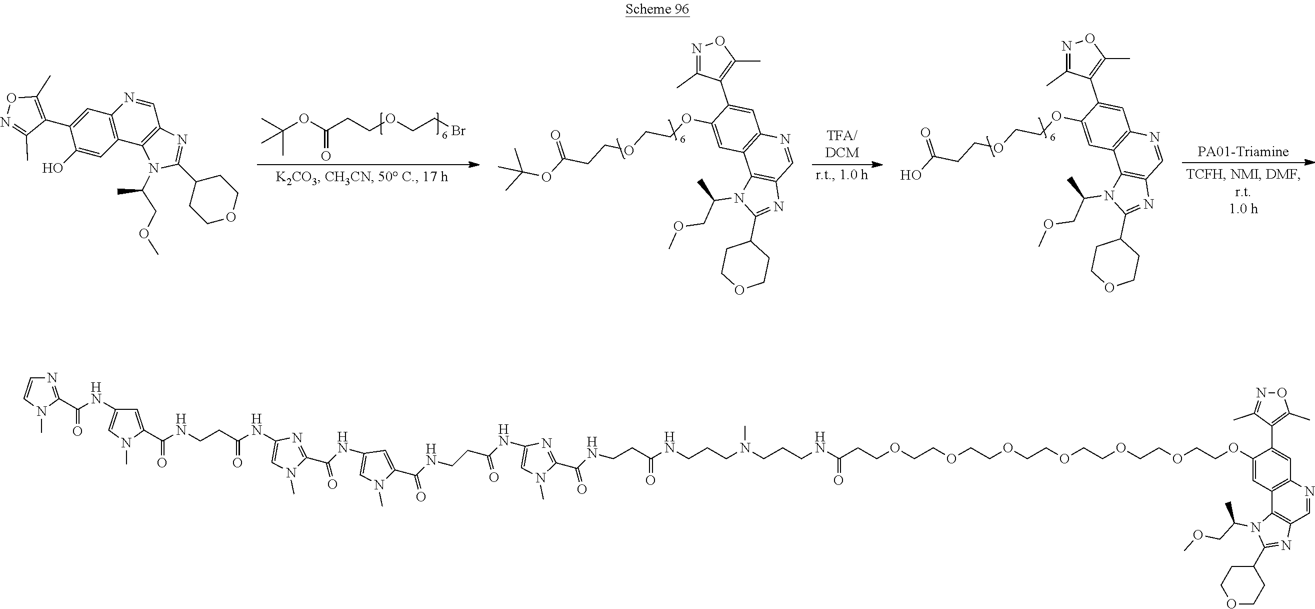

### Step 1: Synthesis of tert-butyl 1-[[7-(3,5-dimethyl-1,2-oxazol-4-yl)-1-[(2R)-1-methoxypropan-2-yl]-2-(oxan-4-yl)imidazo[4,5-c]quinolin-8-yl]oxy]-3,6,9,12,15,18-hexaoxahenicosan-21-oate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate, but the reaction temperature was 50 degree C. 100.00 mg of 7-(3,5-dimethyl-1,2-oxazol-4-yl)-1-[(2R)-1-methoxypropan-2-yl]-2-(oxan-4-yl)imidazo[4,5-c]quinolin-8-ol was used, 70.00 mg of tert-butyl 1-[[17-(3,5-dimethyl-1,2-oxazol-4-yl)-1-[(2R)-1-methoxypropan-2-yl]-2-(oxan-4-yl)imidazo[4,5-c]quinolin-8-yl]oxy]-3,6,9,12,15,18-hexaoxahenicosan-21-oate was obtained as yellow oil (32.55% yield). LC/MS: mass calcd. For C₄₃H₆₄N₄O₁₂: 828.45, found: 851.75 [M+Na].

### Step 2: Synthesis of 1-[[7-(3,5-dimethyl-1,2-oxazol-4-yl)-1-[(2R)-1-methoxypropan-2-yl]-2-(oxan-4-yl)imidazo[4,5-c]quinolin-8-yl]oxy]-3,6,9,12,15,18-hexaoxahenicosan-21-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 60.00 mg of tert-butyl 1-[[7-(3,5-dimethyl-1,2-oxazol-4-yl)-1-[(2R)-1-methoxypropan-2-yl]-2-(oxan-4-yl)imidazo[4,5-c]quinolin-8-yl]oxy]-3,6,9,12,15,18-hexaoxahenicosan-21-oate was used, 60.00 mg crude of 1-[[7-(3,5-dimethyl-1,2-oxazol-4-yl)-1-[(2R)-1-methoxypropan-2-yl]-2-(oxan-4-yl)imidazo[4,5-c]quinolin-8-yl]oxy]-3,6,9,12,15,18-hexaoxahenicosan-21-oic acid was obtained as yellow oil. LC/MS: mass calcd. For C₃₉H₅₆N₄O₁₂: 772.39, found: 773.60 [M+H].

### Step 3: Synthesis of N-[5-1(2-[[2-([2-[(3-[[3-(1-[[7-(3,5-dimethyl-1,2-oxazol-4-yl)-1-[(2R)-1-methoxypropan-2-yl]-2-(oxan-4-yl)imidazo[4,5-c]quinolin-8-yl]oxyl-3,6,9,12,15,18-hexaoxahenicosan-21-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 60.00 mg of 1-[[7-(3,5-dimethyl-1,2-oxazol-4-yl)-1-[(2R)-1-methoxypropan-2-yl]-2-(oxan-4-yl)imidazo[4,5-c]quinolin-8-yl]oxy]-3,6,9,12,15,18-hexaoxahenicosan-21-oic acid was used, 23.50 mg of N-[5-[(2-[[2-([2-[(3-[[3-(1-[[7-(3,5-dimethyl-1,2-oxazol-4-yl)-1-[(2R)-1-methoxypropan-2-yl]-2-(oxan-4-yl)imidazo[4,5-c]quinolin-8-yl]oxy]-3,6,9,12,15,18-hexaoxahenicosan-21-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as white solid (17.15% yield). HRMS: mass calcd. For C₈₂H₁₁₄N₂₂O₁₉: 1710.8631, found: 1711.8661 [M+H]⁺.

### (R)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 27)

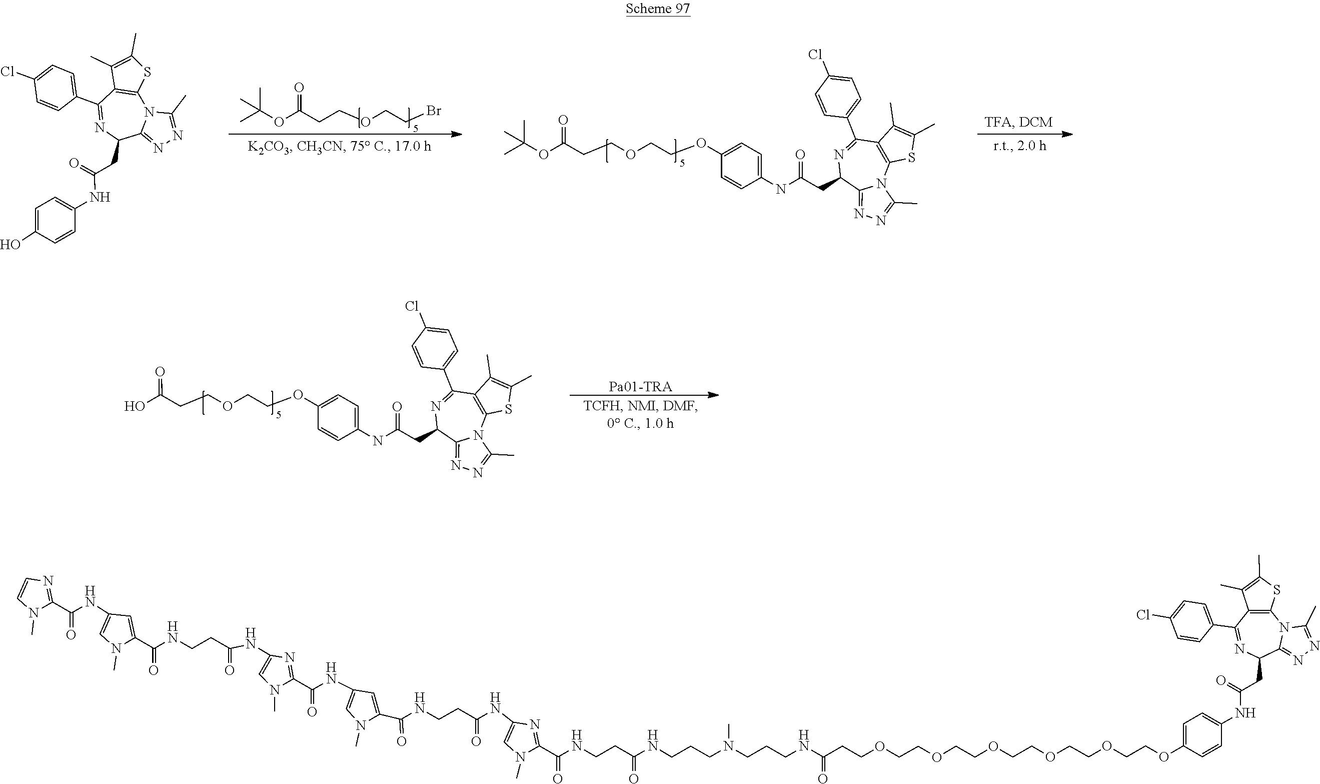

### Step 1: Synthesis of tert-butyl (R)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate, but the reaction temperature was 75 degree C. 80.00 mg of (R)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 100.00 mg of tert-butyl (R)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oate was obtained as yellow oil (73.18% yield). LCMS: mass calcd. For C₄₂H₅₄ClN₅O₉S: 839.33, found: 840.50[M+H]⁺.

### Step 2: Synthesis of (R)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 100.00 mg of tert-butyl (R)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oate was used, 100.00 mg crude of (R)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid was obtained as yellow oil. LCMS: mass calcd. For C₃₈H₄₆ClN₅O₉S: 783.27, found: 784.30 [M+H]⁺.

### Step 3: Synthesis of (R)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 100.00 mg of (R)-1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid was used, 10.40 mg of (R)—N-(5-((3-((2-((1-(4-(2-(4-(4-chlorophenyl)-2,3,9-trimethyl-6H-thieno [3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (4.65% yield). HRMS: mass calcd. For C₈₁H₁₀₄ClN₂₃O₁₆S: 1721.7441, found: 1722.7570 [M+H]⁺.

### (S)—N-(5-((3-((2-((1-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 62)

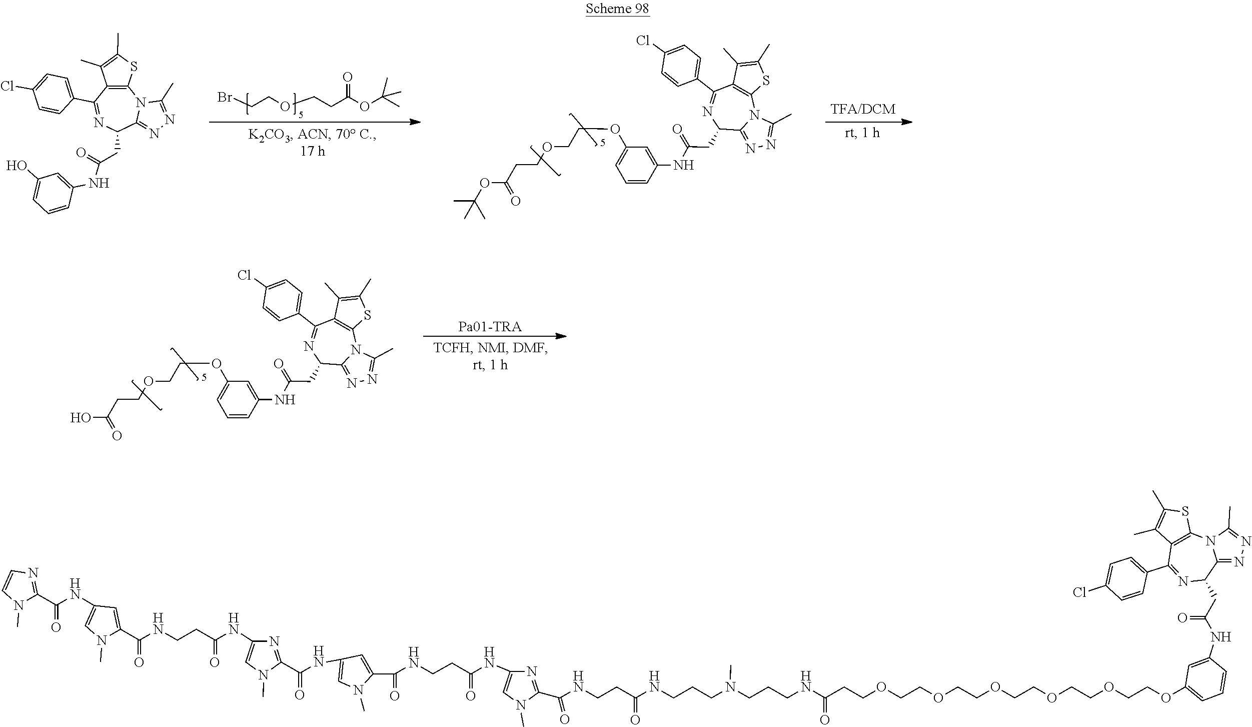

### Step 1: Synthesis of tert-butyl (S)-1-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 100.00 mg of (S)-2-(4-(4-chlorophenyl)-2,3,9-trimethyl-6H-thieno[3,2-f][1,2,4]triazolo[4,3-a][1,4]diazepin-6-yl)-N-(3-hydroxyphenyl)acetamide was used, 140.00 mg crude of tert-butyl (S)-1-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oate was obtained as colorless oil. LCMS: mass calcd. For C₄₂H₅₄ClN₅O₉S: 839.33, found: 862.55 [M+Na]⁺.

### Step 2: Synthesis of (S)-1-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 130.00 mg of tert-butyl (S)-1-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oate was used, 130.00 mg crude of (S)-1-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid was obtained as yellow oil. LC/MS: mass calcd. For C₃₈H₄₆ClN₅O₉S: 783.23, found: 784.50 [M+H]⁺.

### Step 3: Synthesis of (S)—N-(5-((3-((2-((1-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 130.00 mg of (S)-1-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15-pentaoxaoctadecan-18-oic acid was used, 21.40 mg of (S)—N-(5-((3-((2-((1-(3-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (8.12% yield). HRMS: mass calcd. For C₈₁H₁₀₄ClN₂₃O₁₆S: 1721.7441, found: 1722.7520 [M+H]⁺.

### N-[5-[(2-[[2-([2-[(3-[[3-(1-[4-[2-(1,5-dimethyl-6-oxopyridazin-3-yl)-1-[(1S)-1-phenylethyl]imidazo[4,5-c]pyridin-6-yl]piperidin-1-yl]-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide (Compound 9)

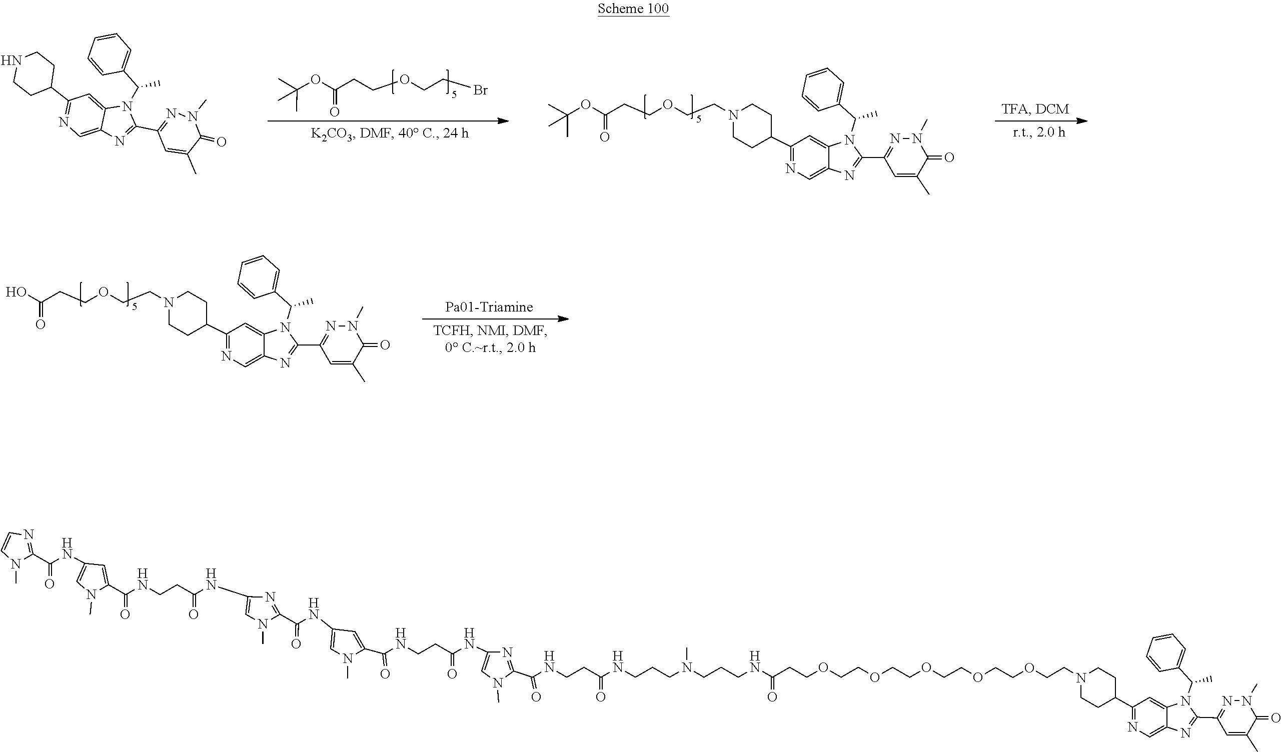

### Step 1: Synthesis of Tert-butyl 1-[4-[2-(1,5-dimethyl-6-oxopyridazin-3-yl)-1-[(1S)-1-phenylethyl]imidazo[4,5-c]pyridin-6-yl]piperidin-1-yl]-3,6,9,12,15-pentaoxaoctadecan-18-oate

The procedure was the same as tert-butyl (S)-(23-(4-(4-(2-(4-(4-chlorophenyl)-2,3,9-trimethyl-6H-thieno[3,2-f][1,2,4]triazolo[4,3-a][1,4]diazepin-6-yl)acetamido)phenyl)piperazin-1-yl)-3,6,9,12,15,18,21-heptaoxatricosyl)carbamate. 80.00 mg of 2,4-dimethyl-6-[1-[(1S)-1-phenylethyl]-6-(piperidin-4-yl)imidazo[4,5-c]pyridin-2-yl]pyridazin-3-one was used, 140.00 mg of tert-butyl 1-[4-[2-(1,5-dimethyl-6-oxopyridazin-3-yl)-1-[(1S)-1-phenylethyl]imidazo[4,5-c]pyridin-6-yl]piperidin-1-yl]-3,6,9,12,15-pentaoxaoctadecan-18-oate was obtained as yellow oil (94.29% yield). LC/MS: mass calcd. For C₄₂H₆₀N₆O₈: 776.45, found: 777.65 [M+H]⁺.

### Step 2: Synthesis of 1-[4-[2-(1,5-Dimethyl-6-oxopyridazin-3-yl)-1-[(1S)-1-phenylethyl]imidazo[4,5-c]pyridin-6-yl]piperidin-1-yl]-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 140.00 mg of tert-butyl 1-[4-[2-(1,5-dimethyl-6-oxopyridazin-3-yl)-1-[(1S)-1-phenylethyl] imidazo[4,5-c]pyridin-6-yl]piperidin-1-yl]-3,6,9,12,15-pentaoxaoctadecan-18-oate was used, 140.00 mg crude of 1-[4-[2-(1,5-Dimethyl-6-oxopyridazin-3-yl)-1-[(1S)-1-phenylethyl]imidazo [4,5-c]pyridin-6-yl]piperidin-1-yl]-3,6,9,12,15-pentaoxaoctadecan-18-oic acid was obtained as yellow oil. LC/MS: mass calcd. For C₃₈H₅₂N₆O₈: 720.38, found: 721.25 [M+H]⁺.

### Step 3: Synthesis of N-[5-[(2-[[2-([2-[(3-[[3-(1-[4-[2-(1,5-dimethyl-6-oxopyridazin-3-yl)-1-[(1S)-1-phenylethyl]imidazo[4,5-c]pyridin-6-yl]piperidin-1-yl]-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 140.00 mg of 1-[4-[2-(1,5-dimethyl-6-oxopyridazin-3-yl)-1-[(1S)-1-phenylethyl]imidazo[4,5-c]pyridin-6-yl]piperidin-1-yl]-3,6,9,12,15-pentaoxaoctadecan-18-oic acid was used, 29.10 mg of N-[5-[(2-[[2-([2-[(3-[[3-(1-[4-[2-(1,5-dimethyl-6-oxopyridazin-3-yl)-1-[(1S)-1-phenylethyl]imidazo[4,5-c]pyridin-6-yl]piperidin-1-yl]-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as white solid (8.81% yield). HRMS: mass calcd. For C₈₁H₁₁₀N₂₄O₁₅: 1658.8582, found: 1659.8629 [M+H]⁺.

### N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide (Compound 10)

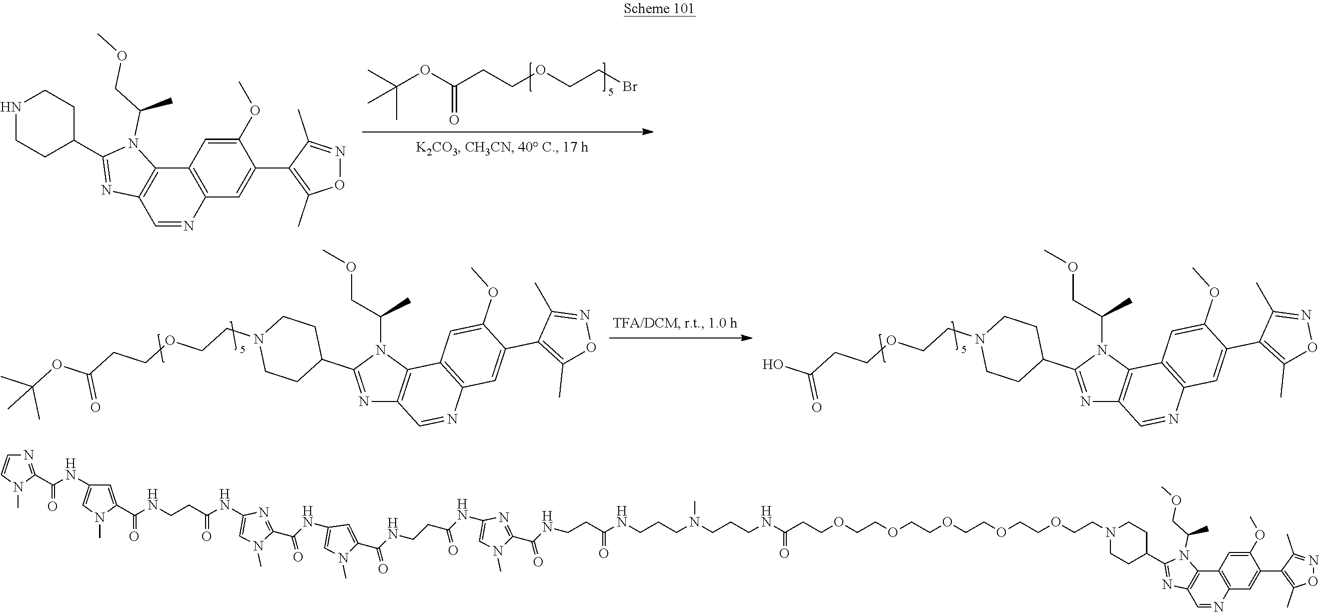

### Step 1: Synthesis of tert-butyl 1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-3,6,9,12,15-pentaoxaoctadecan-18-oate

The procedure was the same as tert-butyl (S)-(23-(4-(4-(2-(4-(4-chlorophenyl)-2,3,9-trimethyl-6H-thieno[3,2-f][1,2,4]triazolo[4,3-a][1,4]diazepin-6-yl)acetamido)phenyl)piperazin-1-yl)-3,6,9,12,15,18,21-heptaoxatricosyl)carbamate. 120.00 mg of 4-[7-(3,5-dimethyl-1,2-oxazol-4-yl)-8-methoxy-1-[(2R)-1-methoxypropan-2-yl]imidazo[4,5-c]quinolin-2-yl]piperidine was used, 120.00 mg of tert-butyl 1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-3,6,9,12,15-pentaoxaoctadecan-18-oate was obtained as yellow oil (53.45% yield). LC/MS: mass calcd. For C₄₂H₆₃N₅O₁₀: 797.46, found: 798.35 [M+H]⁺.

### Step 2: Synthesis of 1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 200.00 mg of tert-butyl 1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-3,6,9,12,15-pentaoxaoctadecan-18-oate was used, 200.00 mg crude of 1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-3,6,9,12,15-pentaoxaoctadecan-18-oic acid was obtained as yellow oil. LC/MS: mass calcd. For C₃₈H₅₅N₅O₁₀: 741.39, found: 742.55 [M+H]⁺.

### Step 3: Synthesis of N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-2,24,34-trioxo-6,9,12,15,18,21-hexaoxa-3,25,29,33-tetraazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 100.00 mg of 1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-3,6,9,12,15-pentaoxaoctadecan-18-oic acid was used, 26.80 mg of N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as white solid (11.64% yield). HRMS: mass calcd. For C₈₁H₁₁₃N₂₃O₁₇: 1679.8685, found: 1680.8708 [M+H]⁺.

### N-[5-([2-[(2-[[2-([3-[(3-[1-[7-(3,5-dimethyl-1,2-oxazol-4-yl)-8-methoxy-2-oxo-1-[(1R)-1-(pyridin-2-yl)ethyl]imidazo[4,5-c]quinolin-3-yl]-3,6,9,12,15-pentaoxaoctadecan-18-amido]propyl)(methyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide (Compound 28)

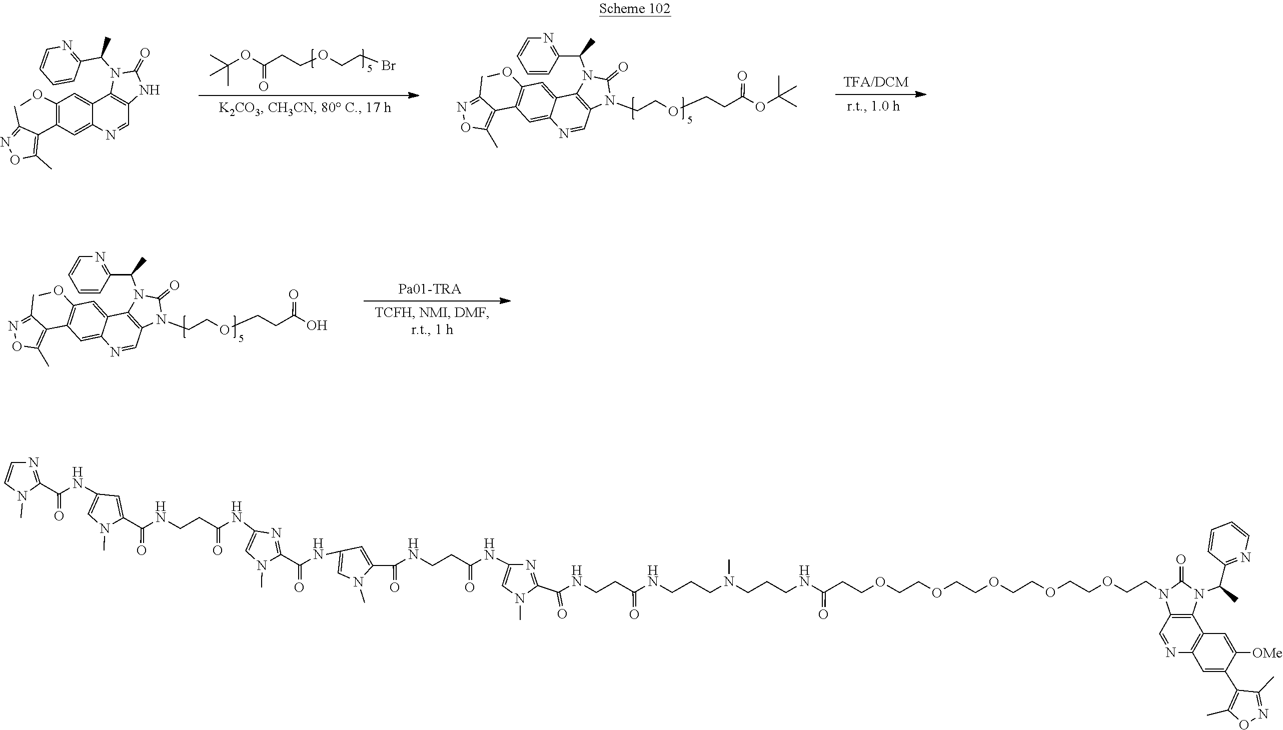

### Step 1: Synthesis of tert-butyl 1-[7-(3,5-dimethyl-1,2-oxazol-4-yl)-8-methoxy-2-oxo-1-[(1R)-1-(pyridin-2-yl)ethyl]imidazo[4,5-c]quinolin-3-yl]-3,6,9,12,15-pentaoxaoctadecan-18-oate

The procedure was the same as tert-butyl (S)-(23-(4-(4-(2-(4-(4-chlorophenyl)-2,3,9-trimethyl-6H-thieno[3,2-f][1,2,4]triazolo[4,3-a][1,4]diazepin-6-yl)acetamido)phenyl)piperazin-1-yl)-3,6,9,12,15,18,21-heptaoxatricosyl)carbamate. 140.00 mg of 7-(3,5-dimethyl-1,2-oxazol-4-yl)-8-methoxy-1-[(1R)-1-(pyridin-2-yl)ethyl]-3H-imidazo[4,5-c]quinolin-2-one was used, 130.00 mg of tert-butyl 1-[7-(3,5-dimethyl-1,2-oxazol-4-yl)-8-methoxy-2-oxo-1-[(1R)-1-(pyridin-2-yl)ethyl]imidazo[4,5-c]quinolin-3-yl]-3,6,9,12,15-pentaoxaoctadecan-18-oate was obtained as yellow oil (49.54% yield). LC/MS: mass calcd. For C₄₀H₅₃N₅O₁₀: 763.38, found: 764.40 [M+H]⁺.

### Step 2: Synthesis of 1-[7-(3,5-dimethyl-1,2-oxazol-4-yl)-8-methoxy-2-oxo-1-[(1R)-1-(pyridin-2-yl)ethyl]imidazo[4,5-c]quinolin-3-yl]-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 130.00 mg of tert-butyl 1-[7-(3,5-dimethyl-1,2-oxazol-4-yl)-8-methoxy-2-oxo-1-[(1R)-1-(pyridine-2-yl)ethyl]imidazo[4,5-c]quinolin-3-yl]-3,6,9,12,15-pentaoxaoctadecan-18-oate was used, 130.00 mg crude of 1-[7-(3,5-dimethyl-1,2-oxazol-4-yl)-8-methoxy-2-oxo-1-[(1R)-1-(pyridin-2-yl)ethyl]imidazo[4,5-c]quinolin-3-yl]-3,6,9,12,15-pentaoxaoctadecan-18-oic acid was obtained as yellow oil. LC/MS: mass calcd. For C₃₆H₄₅N₅O₁₀: 707.32, found: 708.50 [M+H]⁺.

### Step 3: Synthesis of N-[5-([2-[(2-[[2-([3-[(3-[1-[7-(3,5-dimethyl-1,2-oxazol-4-yl)-8-methoxy-2-oxo-1-[(1R)-1-(pyridin-2-yl)ethyl]imidazo[4,5-c]quinolin-3-yl]-3,6,9,12,15-pentaoxaoctadecan-18-amido]propyl)(methyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 130.00 mg of 1-[7-(3,5-dimethyl-1,2-oxazol-4-yl)-8-methoxy-2-oxo-1-[(1R)-1-(pyridin-2-yl)ethyl]imidazo[4,5-c]quinolin-3-yl]-3,6,9,12,15-pentaoxaoctadecan-18-oic acid was used, 52.20 mg of N-[5-([2-[(2-[[2-([3-[(3-[1-[7-(3,5-dimethyl-1,2-oxazol-4-yl)-8-methoxy-2-oxo-1-[(1R)-1-(pyridin-2-yl)ethyl]imidazo[4,5-c]quinolin-3-yl]-3,6,9,12,15-pentaoxaoctadecan-18-amido]propyl)(methyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as white solid (16.96% yield). HRMS: mass calcd. For C₇₉H₁₀₃N₂₃O₁₇: 1645.79, found: 1646.7905 [M+H]⁺.

### N-[5-[(2-[[2-([2-[(3-[[3-(1-[4-[2-(1,5-dimethyl-6-oxopyridazin-3-yl)-1-[(1S)-1-phenylethyl]imidazo[4,5-c]pyridin-6-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide (Compound 19)

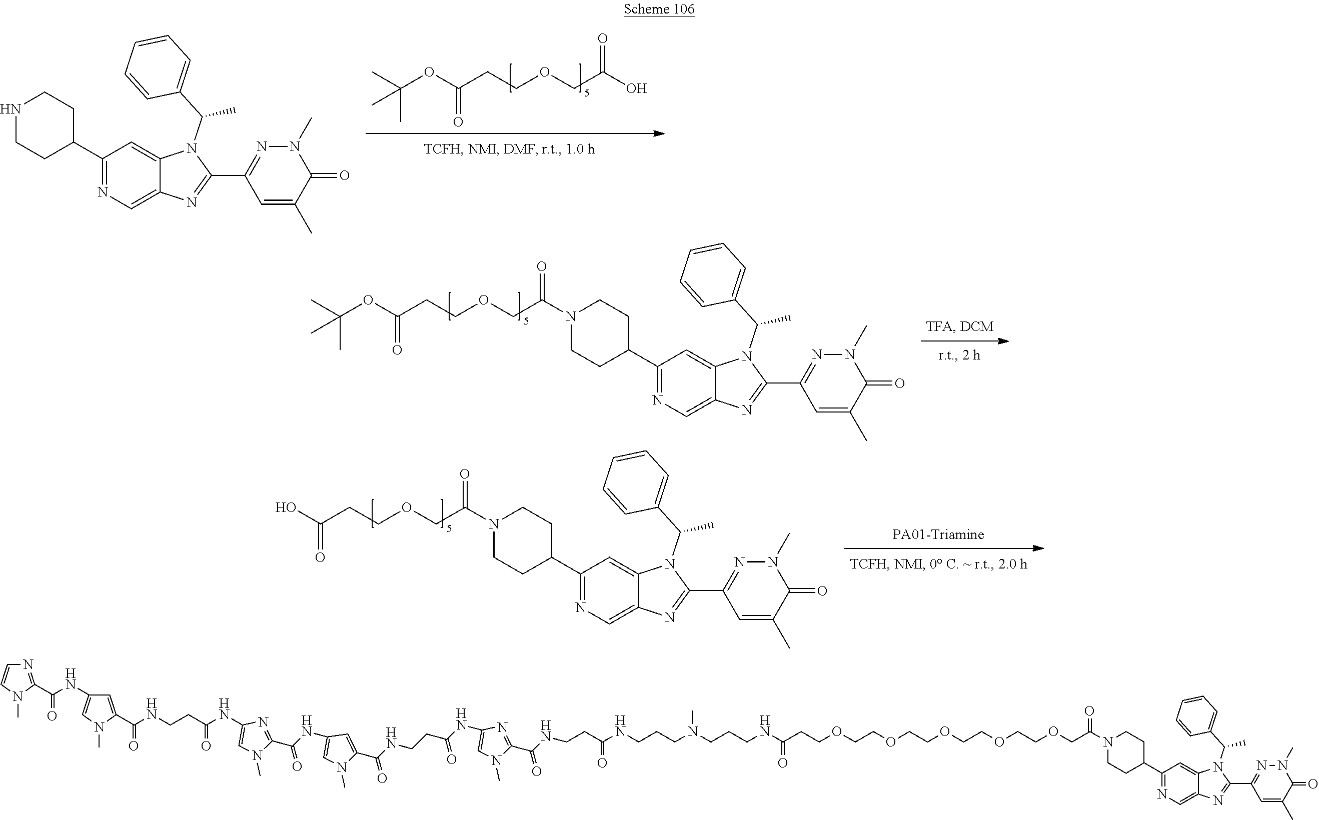

### Step 1: Synthesis of Tert-butyl 1-[4-[2-(1,5-dimethyl-6-oxopyridazin-3-yl)-1-[(1S)-1-phenylethyl]imidazo[4,5-c]pyridin-6-yl]piperidin-1-yl]-1-oxo-3,6,9,12,15-pentaoxaoctadecan-18-oate

The procedure was the same as tert-butyl 1-[[4-([[3-(4-methanesulfonyl-2-[6-methyl-7-oxo-1H-pyrrolo[2,3-c]pyridin-4-yl]phenoxy)phenyl]amino]methyl)phenyl]carbamoyl]-2,5,8,11,14-pentaoxaheptadecan-17-oate. 120.00 mg of 2,4-dimethyl-6-[1-[(1S)-1-phenylethyl]-6-(piperidin-4-yl)imidazo[4,5-c]pyridin-2-yl]pyridazin-3-one was used, 82.00 mg of tert-butyl 1-[4-[2-(1,5-dimethyl-6-oxopyridazin-3-yl)-1-[(1S)-1-phenylethyl]imidazo[4,5-c]pyridin-6-yl]piperidin-1-yl]-1-oxo-3,6,9,12,15-pentaoxaoctadecan-18-oate was obtained as colorless oil (33.32% yield). LC/MS: mass calcd. For C₄₂H₅₈N₆O₉: 790.43, found: 791.50 [M+H]⁺.

### Step 2: Synthesis of 1-[4-[2-(1,5-Dimethyl-6-oxopyridazin-3-yl)-1-[(1S)-1-phenylethyl]imidazo[4,5-c]pyridin-6-yl]piperidin-1-yl]-1-oxo-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 77.00 mg of tert-butyl 1-[4-[2-(1,5-dimethyl-6-oxopyridazin-3-yl)-1-[(1S)-1-phenylethyl][4,5-c]pyridin-6-yl]piperidin-1-yl]-1-oxo-3,6,9,12,15-pentaoxaoctadecan-18-oate was used, 75.00 mg crude of 1-[4-[2-(1,5-Dimethyl-6-oxopyridazin-3-yl)-1-[(1S)-1-phenylethyl]imidazo[4,5-c]pyridin-6-yl]piperidin-1-yl]-1-oxo-3,6,9,12,15-pentaoxaoctadecan-18-oic acid was obtained as yellow oil. LC/MS: mass calcd. For C₃₈H₅₀N₆O₉: 734.36, found: 757.45 [M+Na]⁺.

### Step 3: Synthesis of N-[5-[(2-[[2-([2-[(3-[[3-(1-[4-[2-(1,5-dimethyl-6-oxopyridazin-3-yl)-1-[(1S)-1-phenylethyl]imidazo[4,5-c]pyridin-6-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 75.00 mg of 1-[4-[2-(1,5-dimethyl-6-oxopyridazin-3-yl)-1-[(1S)-1-phenylethyl]imidazo[4,5-c]pyridin-6-yl] piperidin-1-yl]-1-oxo-3,6,9,12,15-pentaoxaoctadecan-18-oic acid was used, 20.50 mg of N-[5-[(2-[[2-([2-[(3-[[3-(1-[4-[2-(1,5-dimethyl-6-oxopyridazin-3-yl)-1-[(1S)-1-phenylethyl]imidazo[4,5-c]pyridin-6-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as white solid (11.56% yield). HRMS: mass calcd. For C₈₁H₁₀₈N₂₄O₁₆: 1672.8375, found: 1673.8425 [M+H]⁺

### N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide (Compound 20)

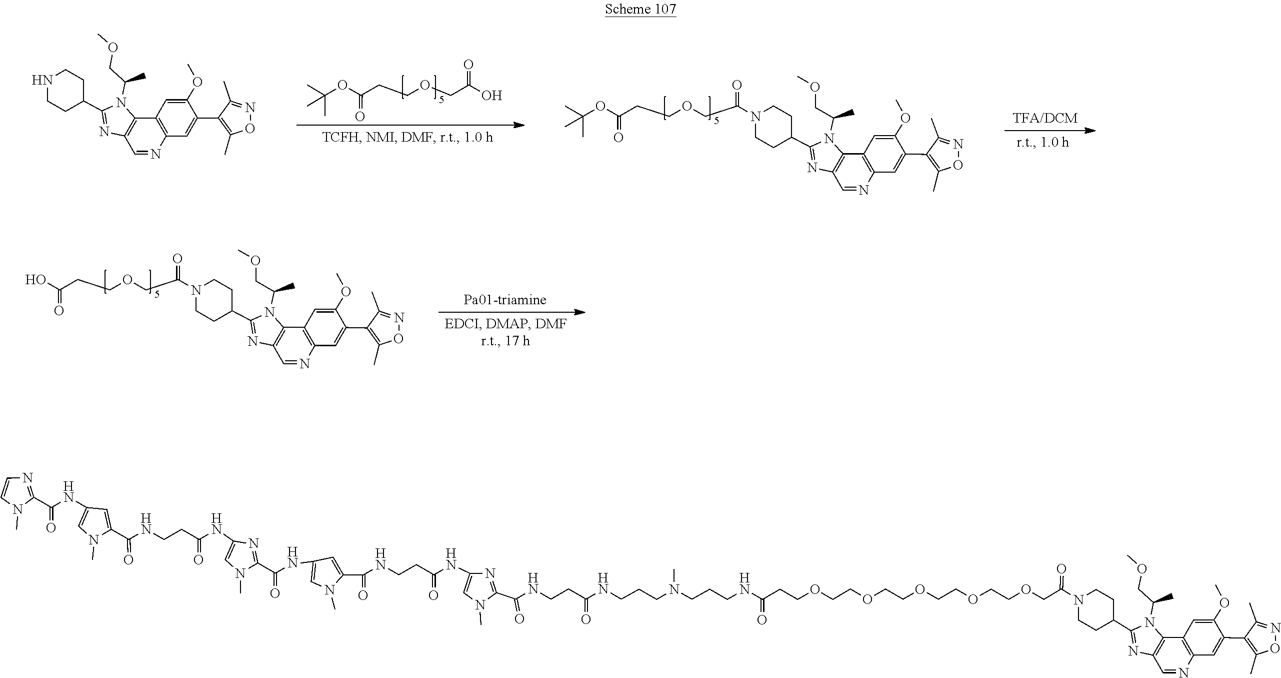

### Step 1: Synthesis of tert-butyl 1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-oate

The procedure was the same as tert-butyl 1-[[4-([[3-(4-methanesulfonyl-2-[6-methyl-7-oxo-1H-pyrrolo[2,3-c]pyridin-4-yl]phenoxy)phenyl]amino]methyl)phenyl]carbamoyl]-2,5,8,11,14-pentaoxaheptadecan-17-oate. 120.00 mg of 4-[7-(3,5-dimethyl-1,2-oxazol-4-yl)-8-methoxy-1-[(2R)-1-methoxypropan-2-yl]imidazo[4,5-c]quinolin-2-yl]piperidine was used, 150.00 mg of tert-butyl 1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-oate was obtained as yellow oil (67.23% yield). LC/MS: mass calcd. For C₄₂H₆₁N₅O₁₁: 811.44, found: 812.60 [M+H]⁺.

### Step 2: Synthesis of 1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 140.00 mg of tert-butyl 1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-oate was used, 150.00 mg crude of 1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-oic acid was obtained as yellow oil. LC/MS: mass calcd. For C₃₈H₅₃N₅O₁₁: 755.37, found: 778.50 [M+Na]⁺.

### Step 3: Synthesis of N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c] quinolin-2-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide

Into a 25 mL flask was added 1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-oic acid (100.00 mg, 0.13 mmol, 1.00 equiv), DMF (5.00 mL), N-[5-([2-[(2-[[2-([3-[(3-aminopropyl)(methyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide (126.62 mg, 0.13 mmol, 1.00 equiv), EDCI (38.04 mg, 0.20 mmol, 1.50 equiv), the mixture was stirred at room temperature for 5 mins, then DMAP (40.41 mg, 0.33 mmol, 2.50 equiv) was added. The reaction was stirred at room temperature for 17 h. The reaction mixture was filtered and the filtration in DMF (5.5 mL) was purified by Prep-HPLC with the following conditions: Column: Xselect CSH F-Phenyl OBD column, 19*250, 5 um; Mobile Phase A: Water (0.05% TFA), Mobile Phase B: ACN; Flow rate: 25 mL/min; Gradient: 50 B to 57 B in 10 min; 254 nm; RT1: 9. The fractions were combined and lyophilized directly. This resulted in N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide (43.3 mg, 19.27%) as white solid. HRMS: mass calcd. For C₈₁H₁₁₁N₂₃O₁₈: 1693.8477, found: 1694.8518 [M+H]⁺.

### N-[5-[(2-[[2-([2-[(3-[[3-(1-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide (Compound 29)

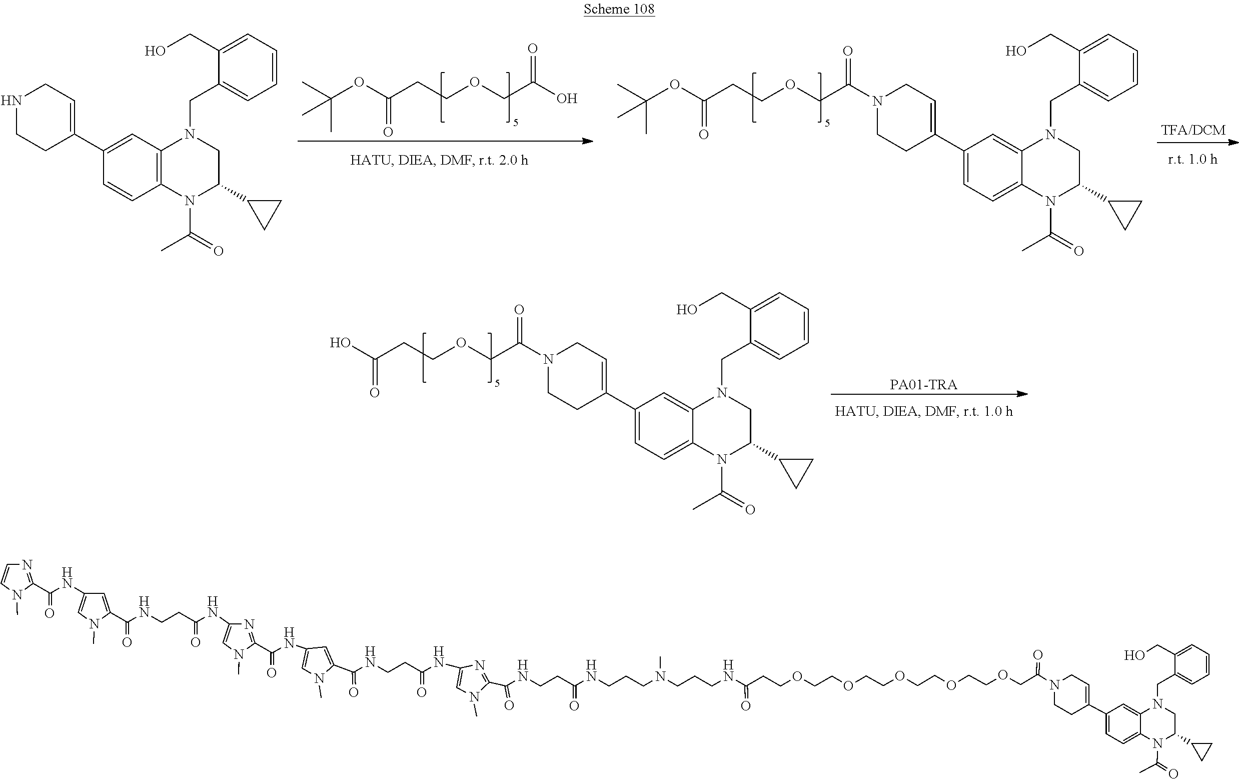

### Step 1: Synthesis of tert-butyl 1-[4-[(2S)-1-acetyl-2-cyclopropyl-4-[[2-(hydroxymethyl) phenyl]methyl]-2,3-dihydroquinoxalin-6-yl]-3,6-dihydro-2H-pyridin-1l-yl]-1-oxo-3,6,9,12,15-pentaoxaoctadecan-18-oate

The procedure was the same as tert-butyl N-(23-[4-[7-(3,5-dimethyl-1,2-oxazol-4-yl)-8-methoxy-1l-[(2R)-1-methoxypropan-2-yl] imidazo [4,5-c] quinolin-2-yl] piperidin-1-yl]-23-oxo-3,6,9,12,15,18,21-heptaoxatricosan-1-yl)carbamate. 170.00 mg of 1-[(2S)-2-cyclopropyl-4-[[2-(hydroxymethyl)phenyl] methyl]-6-(1, 2, 3,6-tetrahydropyridin-4-yl)-2,3-dihydroquinoxalin-1l-yl]ethanone was used, 170.00 mg of tert-butyl 1-[4-[(2S)-1-acetyl-2-cyclopropyl-4-[[2-(hydroxymethyl)phenyl] methyl]-2,3-dihydroquinoxalin-6-yl]-3,6-dihydro-2H-pyridin-1-yl]-1-oxo-3,6,9,12,15-pentaoxaoctadecan-18-oate was obtained as yellow oil (47.58% yield). LC/MS: mass calcd. For C₄₃H₆₁N₃O₁₀: 779.44, found: 780.60 [M+H]⁺.

### Step 2: Synthesis of 1-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-1-oxo-3,6,9,12,15-pentaoxaoctadecan-18-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 160.00 mg of tert-butyl 1-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1l-yl]-1-oxo-3,6,9,12,15-pentaoxaoctadecan-18-oate was used, 160.00 mg crude of 1-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-1-oxo-3,6,9,12,15-pentaoxaoctadecan-18-oic acid was obtained as yellow oil. LC/MS: mass calcd. For C₃₉H₅₃N₃O₁₀: 723.37, found: 724.50 [M+H]⁺.

### Step 3: Synthesis of N-[5-[(2-[[2-([2-[(3-[[3-(1-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-1-oxo-3,6,9,12,15-pentaoxaoctadecan-18-amido)propyl] (methyl)amino] propyl)carbamoyl] ethyl] carbamoyl)-1-methylimidazol-4-yl] carbamoyl] ethyl)carbamoyl]-1-methylpyrrol-3-yl]-1-methyl-4-(3-[[1-methyl-4-(1-methylimidazole-2-amido)pyrrol-2-yl] form amido] propanamido)imidazole-2-carboxamide

The procedure was the same as (S)—N-(5-((3-((2-((1-(4-(4-chlorophenyl)-2,3,9-trimethyl-6H-thieno[3,2-f][1,2,4]triazolo [4,3-a][1,4]diazepin-6-yl)-29-methyl-2,24,34-trioxo-6,9,12,15,18,21-hexaoxa-3,25,29,33-tetraazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 160.00 mg of 1-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-1-oxo-3,6,9,12,15-pentaoxaoctadecan-18-oic acid was used, 28.10 mg of N-[5-[(2-[[2-([2-[(3-[[3-(1-[4-[(2S)-1-acetyl-2-cyclopropyl-4-[[2-(hydroxymethyl)phenyl]methyl]-2,3-dihydroquinoxalin-6-yl]-3,6-dihydro-2H-pyridin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as white solid (7.43% yield). HRMS: mass calcd. For C₈₂H₁₁₁N₂₁O₁₇: 1661.8467, found: 1662.8562 [M+H]⁺.

Type 9 Synthetic Route to Transcription Modulator Molecules

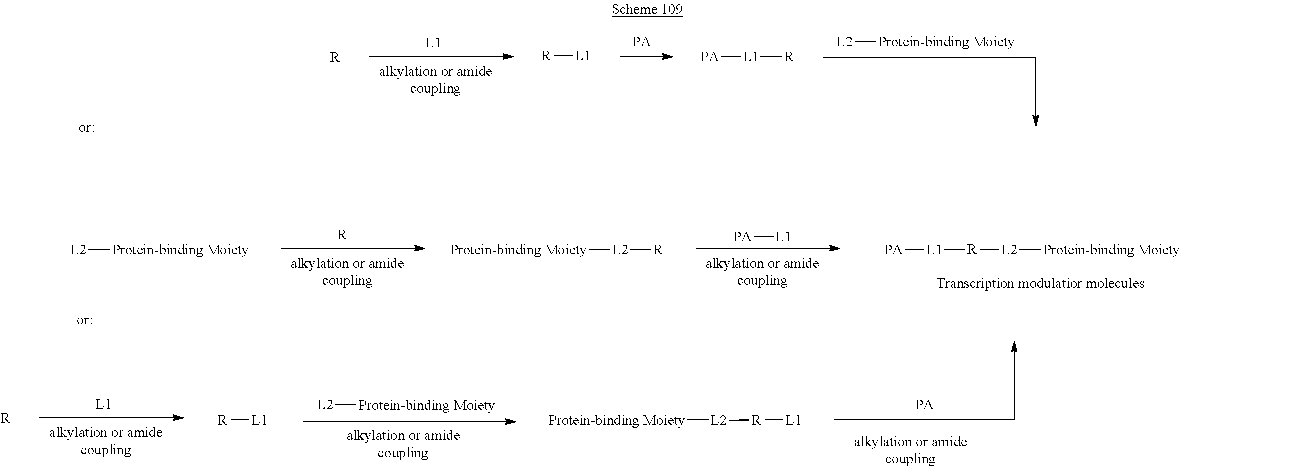

### (S)—N-(5-((3-((2-((1-(4-(2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)acetyl)piperazin-1-yl)-13-oxo-3,6,9-trioxa-12-azapentadecan-15-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 39)

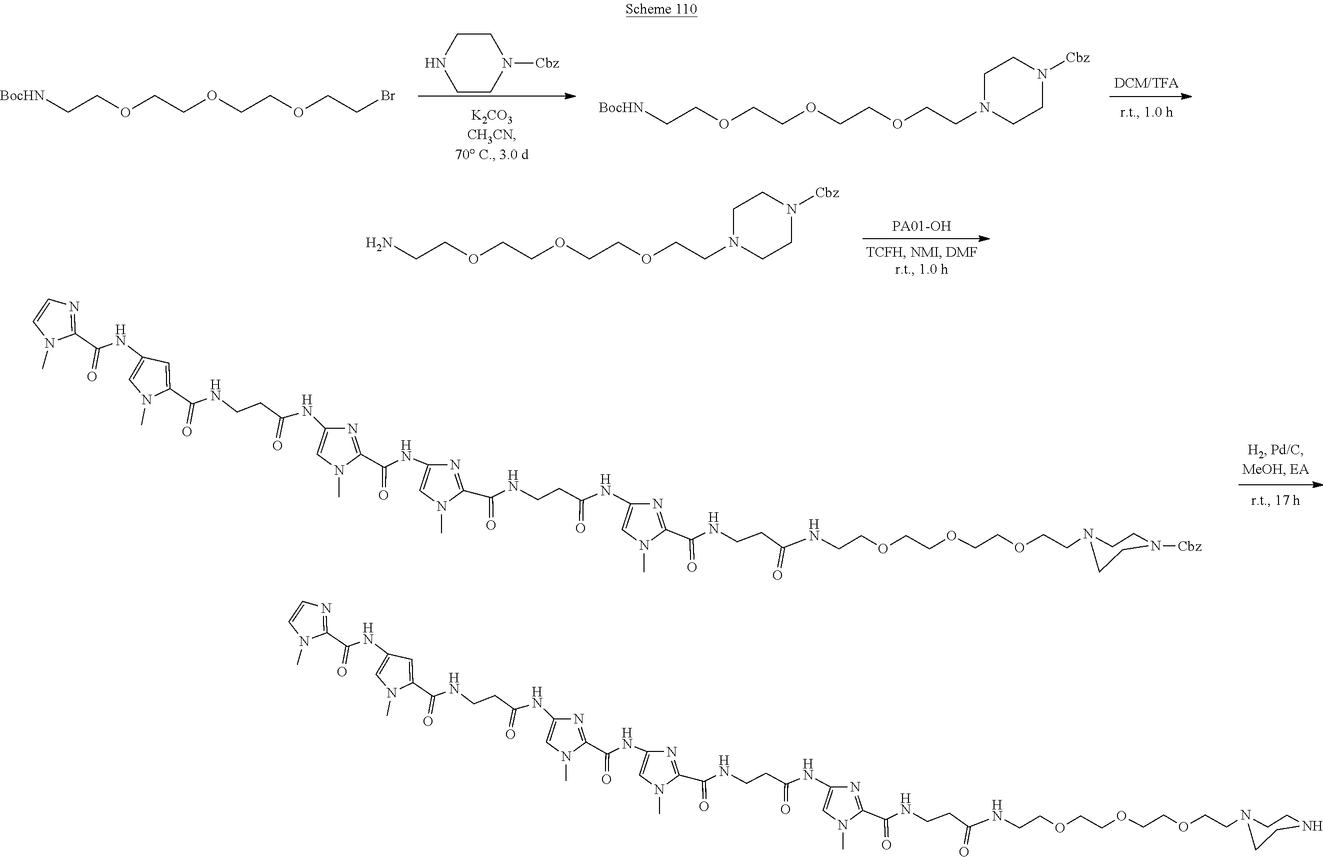 [SMILES: CC(C)(C)OC(=O)NCCOCCOCCOCCBr ; O=C(OCc1ccccc1)N1CCNCC1 ; CC(C)(C)OC(=O)NCCOCCOCCOCCN1CCN(C(=O)OCc2ccccc2)CC1 ; NCCOCCOCCOCCN1CCN(C(=O)OCc2ccccc2)CC1 ; Cn1cc(NC(=O)c2nccn2C)cc1C(=O)NCCC(=O)Nc1cn(C)c(C(=O)Nc2cn(C)c(C(=O)NCCC(=O)Nc3cn(C)c(C(=O)NCCC(=O)NCCOCCOCCOCCN4CCN(C(=O)OCc5ccccc5)CC4)n3)n2)n1 ; Cn1cc(NC(=O)c2nccn2C)cc1C(=O)NCCC(=O)Nc1cn(C)c(C(=O)Nc2cn(C)c(C(=O)NCCC(=O)Nc3cn(C)c(C(=O)NCCC(=O)NCCOCCOCCOCCN4CCNCC4)n3)n2)n1]

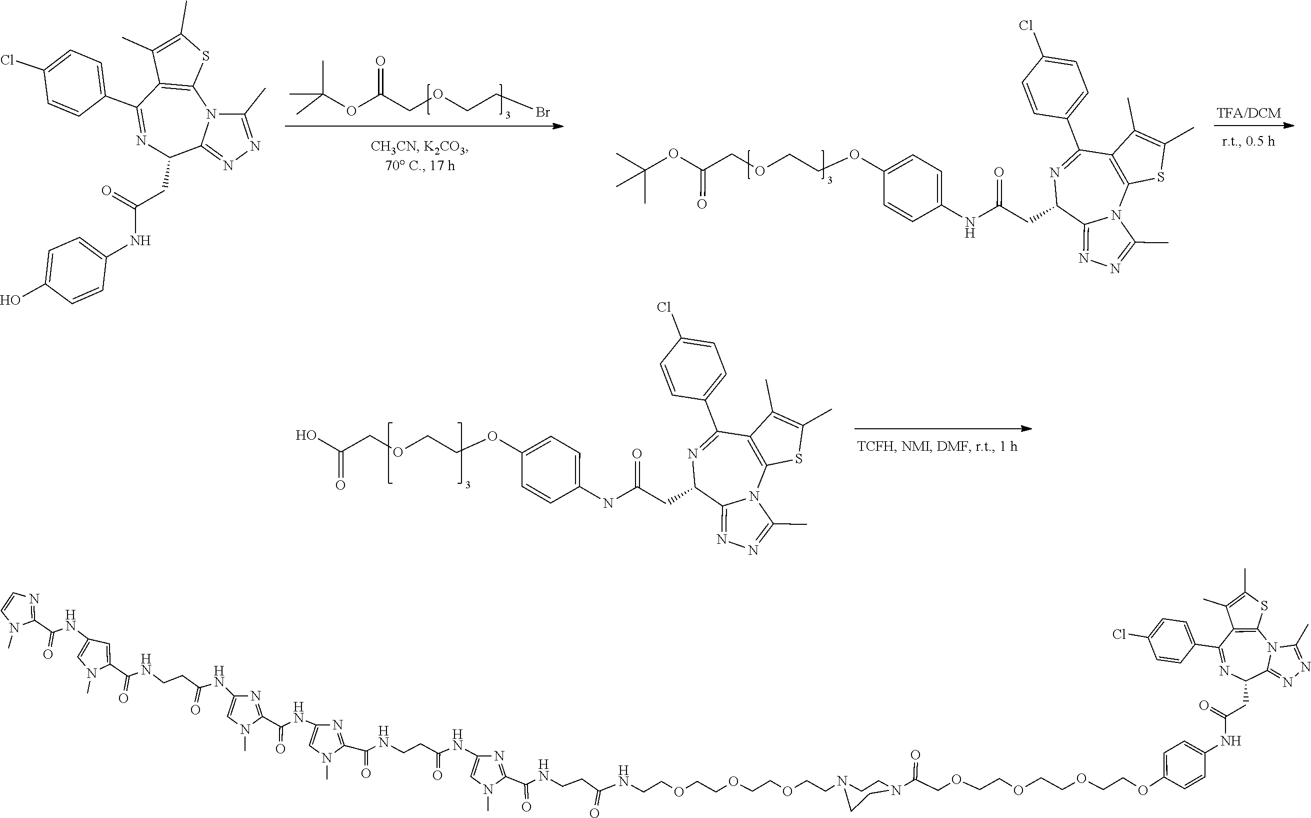

### Step 1: Synthesis of benzyl 4-[2-[2-(2-[2-[(tert-butoxycarbonyl)amino]ethoxy]ethoxy)ethoxy]ethyl]piperazine-1-carboxylate

The procedure was the same as tert-butyl (S)-(23-(4-(4-(2-(4-(4-chlorophenyl)-2,3,9-trimethyl-6H-thieno[3,2-f][1,2,4]triazolo[4,3-a][1,4]diazepin-6-yl)acetamido)phenyl)piperazin-1-yl)-3,6,9,12,15,18,21-heptaoxatricosyl)carbamate, but the reaction time was 3 d. 600.00 mg of tert-butyl N-(2-[2-[2-(2-bromoethoxy)ethoxy]ethoxy]ethyl)carbamate was used, 600.00 mg of benzyl 4-[2-[2-(2-[2-[(tert-butoxycarbonyl)amino]ethoxy]ethoxy)ethoxy]ethyl]piperazine-1-carboxylate was obtained as a yellow oil (71.88% yield). LC/MS: mass calcd. For C₂₅H₄₁N₃O₇: 495.29, found: 496.45 [M+H].

### Step 2: Synthesis of benzyl 4-(2-[2-[2-(2-aminoethoxy)ethoxy]ethoxy]ethyl)piperazine-1-carboxylate

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 300.00 mg of benzyl 4-[2-[2-(2-[2-[(tert-butoxycarbonyl)amino]ethoxy]ethoxy) ethoxy]ethyl]piperazine-1-carboxylate was used, 300.00 mg of benzyl 4-(2-[2-[2-(2-aminoethoxy)ethoxy]ethoxy]ethyl)piperazine-1-carboxylate was obtained as yellow oil. LC/MS: mass calcd. For C₂₀H₃₃N₃O₅: 395.24, found: 396.25 [M+H].

### Step 3: Synthesis of benzyl 4-[12-[2-(2-[12-[3-([1-methyl-4-[3-([1-methyl-4-[1-methyl-4-(3-[[1-methyl-4-(1-methylimidazole-2-amido)pyrrol-2-yl]formamido] propanamido)imidazole-2-amido]pyrrol-2-yl]formamido)propanamido]imidazol-2-yl]formamido)propanamido]ethoxy]ethoxy)ethoxy]ethyl]piperazine-1-carboxylate

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 210.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid was used, 300.00 mg of benzyl 4-[2-[2-(2-[2-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]ethoxy]ethoxy)ethoxy]ethyl]piperazine-1-carboxylate was obtained as yellow oil (75.19% yield). LC/MS: mass calcd. For C₅₆H₇₄N₁₈O₁₃: 1206.57, found: 1207.85 [M+H]⁺.

### Step 4: 1-methyl-4-(3-[[1-methyl-4-(1-methylimidazole-2-amido)pyrrol-2-yl]formamido]propanamido)-N-(1-methyl-5-[[2-([1-methyl-2-[(2-[[2-(2-[2-[2-(piperazin-1-yl)ethoxy]ethoxy]ethoxy)ethyl]carbamoyl]ethyl)carbamoyl]imidazol-4-yl]carbamoyl)ethyl]carbamoyl]pyrrol-3-yl)imidazole-2-carboxamide

Into a 25 mL flask was added benzyl 4-[2-[2-(2-[2-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]ethoxy]ethoxy)ethoxy]ethyl]piperazine-1-carboxylate (150.00 mg, 0.12 mmol, 1.00 equiv), MeOH (2.00 mL), EA (2.00 mL), Pd/C (40.00 mg, 27% w/w). The reaction was stirred at room temperature for 17 h under H₂ atmosphere. The Pd/C was filtered, 1M HCl in MeOH (1.0 mL) was added to the filtrate and the filtrate was concentrated. This resulted in 1-methyl-4-(3-[[1-methyl-4-(1-methylimidazole-2-amido) pyrrol-2-yl]formamido]propanamido)-N-(1-methyl-5-[[2-([1-methyl-2-[(2-[[2-(2-[2-[2-(piperazin-1-yl)ethoxy]ethoxy]ethoxy)ethyl]carbamoyl]ethyl)carbamoyl]imidazol-4-yl]carbamoyl)ethyl]carbamoyl]pyrrol-3-yl)imidazole-2-carboxamide (100 mg, 68.31%) as light green solid. LC/MS: mass calcd. For C₄₈H₆₈N₁₈O₁₁: 1072.53, found: 1095.80 [M+Na]⁺

### Step 5: Synthesis of tert-butyl (S)-2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)acetate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 100.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 150.00 mg of tert-butyl (S)-2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)acetate was obtained as yellow oil (100% yield). LC/MS: mass calcd. For C₃₇H₄₄ClN₅O₇S: 737.26, found: 738.45 [M+H]⁺.

### Step 6: Synthesis of (S)-2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)acetic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 150.00 mg of tert-butyl (S)-2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)acetate was used, 160.00 mg crude of (S)-2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)acetic acid was obtained as yellow oil. LC/MS: mass calcd. For C₃₃H₃₆ClN₅O₇S: 681.20, found: 682.35 [M+H]⁺.

### Step 7: Synthesis of (S)—N-(5-((3-((2-((1-(4-(2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)acetyl)piperazin-1-yl)-13-oxo-3,6,9-trioxa-12-azapentadecan-15-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-23-methyl-18,28-dioxo-3,6,9,12,15-pentaoxa-19,23,27-triazatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 63.57 mg of (S)-2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)acetic acid was used, 8.80 mg of (S)—N-(5-((3-((2-((1-(4-(2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)acetyl)piperazin-1-yl)-13-oxo-3,6,9-trioxa-12-azapentadecan-15-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (5.09% yield). HRMS: mass calcd. For C₈₁H₁₀₂ClN₂₃O₁₇S: 1735.7233, found: 1736.7289 [M+H]⁺.

### (S)—N-(5-((3-((2-((1-(4-(2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)piperazin-1-yl)-13-oxo-3,6,9-trioxa-12-azapentadecan-15-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 40)

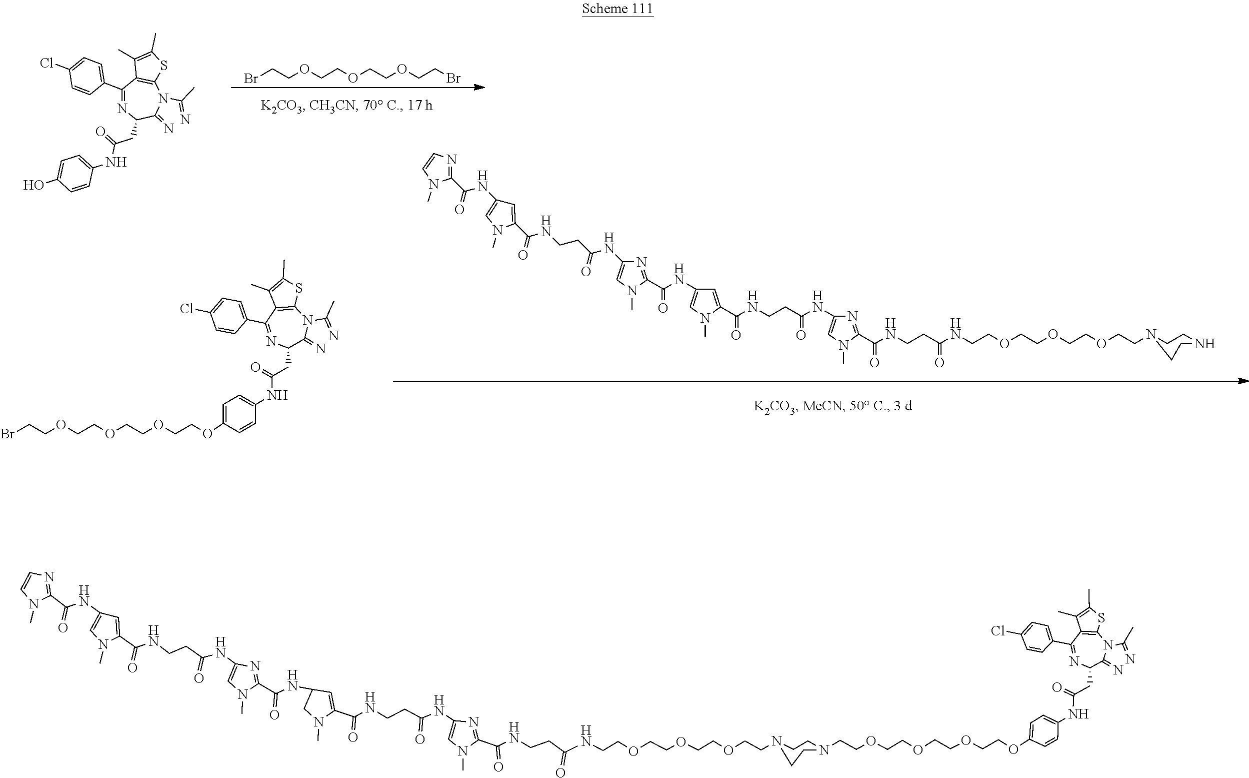

### Step 1: Synthesis of (S)—N-(4-(2-(2-(2-(2-bromoethoxy)ethoxy)ethoxy)ethoxy)phenyl)-2-(4-(4-chlorophenyl)-2,3,9-trimethyl-6H-thieno[3,2-f][1,2,4]triazolo[4,3-a][1,4]diazepin-6-yl)acetamide

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 300.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 240.00 mg of (S)—N-(4-(2-(2-(2-(2-bromoethoxy)ethoxy)ethoxy)ethoxy) phenyl)-2-(4-(4-chlorophenyl)-2,3,9-trimethyl-6H-thieno[3,2-f][1,2,4]triazolo[4,3-a][1,4]diazepin-6-yl)acetamide was obtained as yellow oil (53.84% yield). LC/MS: mass calcd. For C₃₃H₃₇BrClN₅O₅S: 729.14, found: 731.10, 732.30 [M+H, M+2+H]⁺.

### Step 2: Synthesis of (S)—N-(5-((3-((2-((1-(4-(2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)piperazin-1-yl)-13-oxo-3,6,9-trioxa-12-azapentadecan-15-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as tert-butyl (S)-(23-(4-(4-(2-(4-(4-chlorophenyl)-2,3,9-trimethyl-6H-thieno[3,2-f][1,2,4]triazolo[4,3-a][1,4]diazepin-6-yl)acetamido)phenyl)piperazin-1-yl)-3,6,9,12,15,18,21-heptaoxatricosyl)carbamate, but the reaction temperature was decreased to 50 degree C., and the reaction time was 3.0 days. The product was purified by Prep-HPLC. 34.06 mg of (S)—N-(4-(2-(2-(2-(2-bromoethoxy)ethoxy)ethoxy)ethoxy)phenyl)-2-(4-(4-chlorophenyl)-2,3,9-trimethyl-6H-thieno[3,2-f][1,2,4]triazolo[4,3-a][1,4]diazepin-6-yl)acetamide was used, 3.70 mg of (S)—N-(5-((3-((2-((1-(4-(2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)piperazin-1-yl)-13-oxo-3,6,9-trioxa-12-azapentadecan-15-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (4.60% yield). HRMS: mass calcd. For C₈₁H₁₀₄ClN₂₃O₁₆S: 1721.7441, found: 1722.7517 [M+H]⁺.

### (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oic acid (Compound 68)

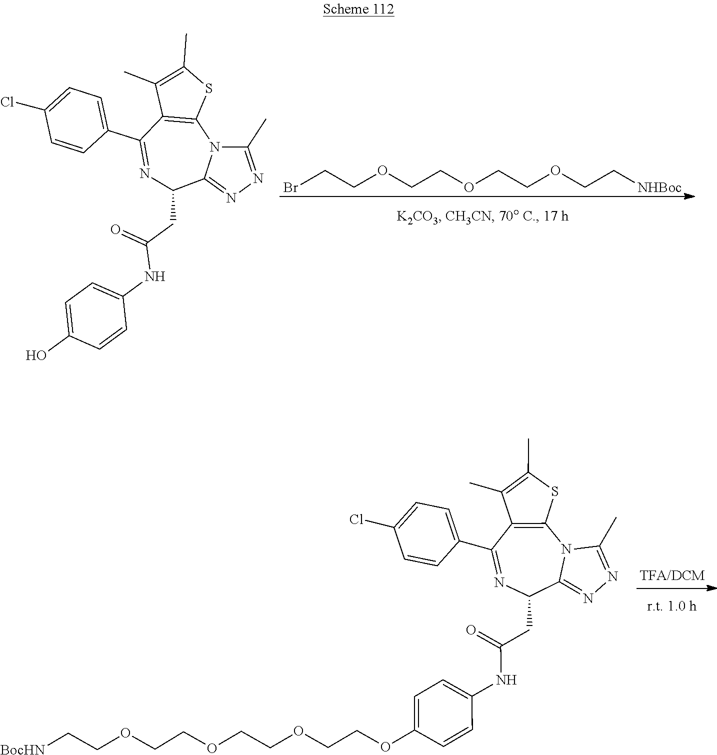

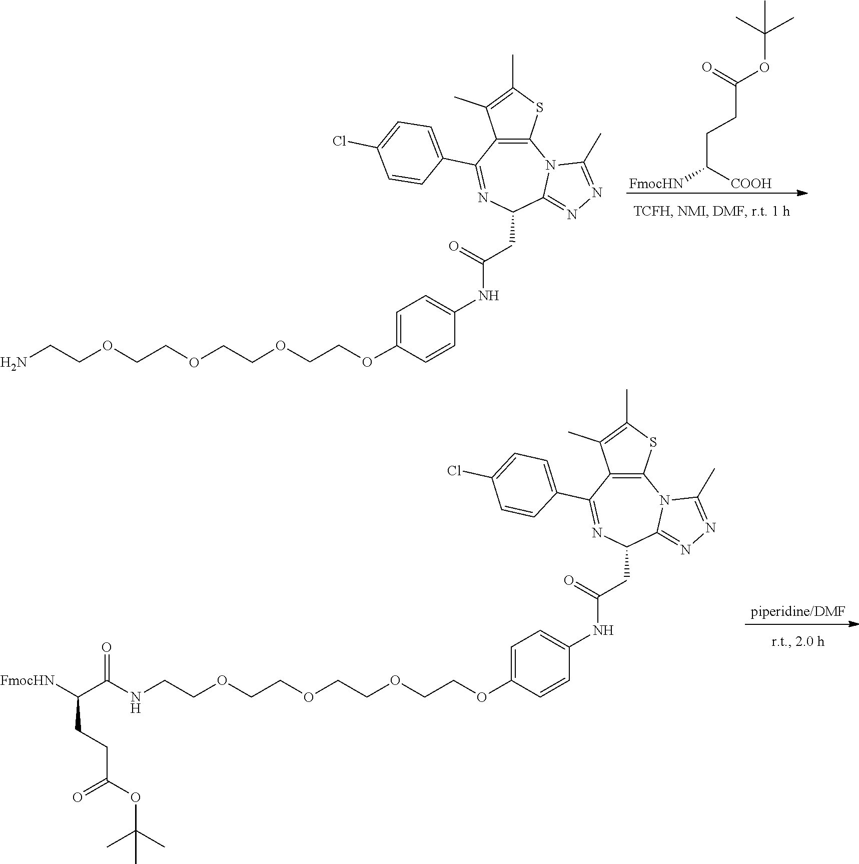

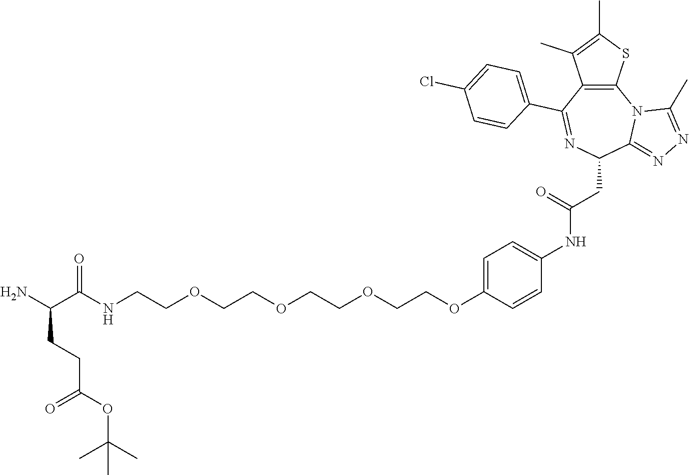 [SMILES: Cc1sc2c(c1C)C(c1ccc(Cl)cc1)=N[C@@H](CC(=O)Nc1ccc(OCCOCCOCCOCCNC(=O)[C@H](N)CCC(=O)OC(C)(C)C)cc1)c1nnc(C)n1-2]

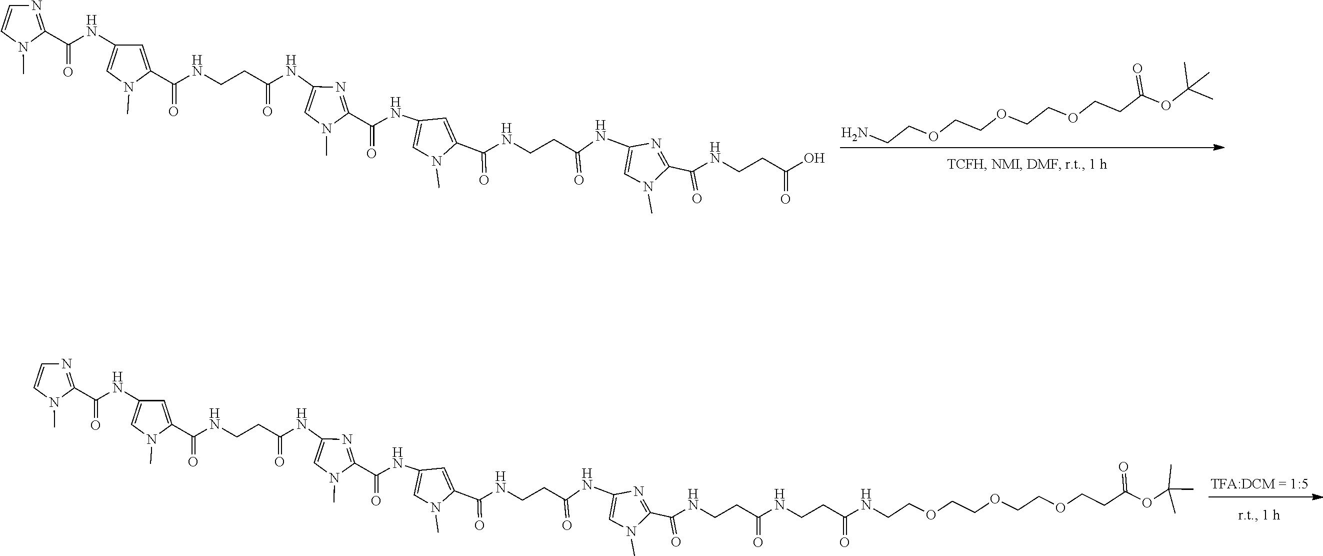 [SMILES: Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)O)n1 ; CC(C)(C)OC(=O)CCOCCOCCOCCN ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCC(=O)NCCOCCOCCOCCC(=O)OC(C)(C)C)n1]

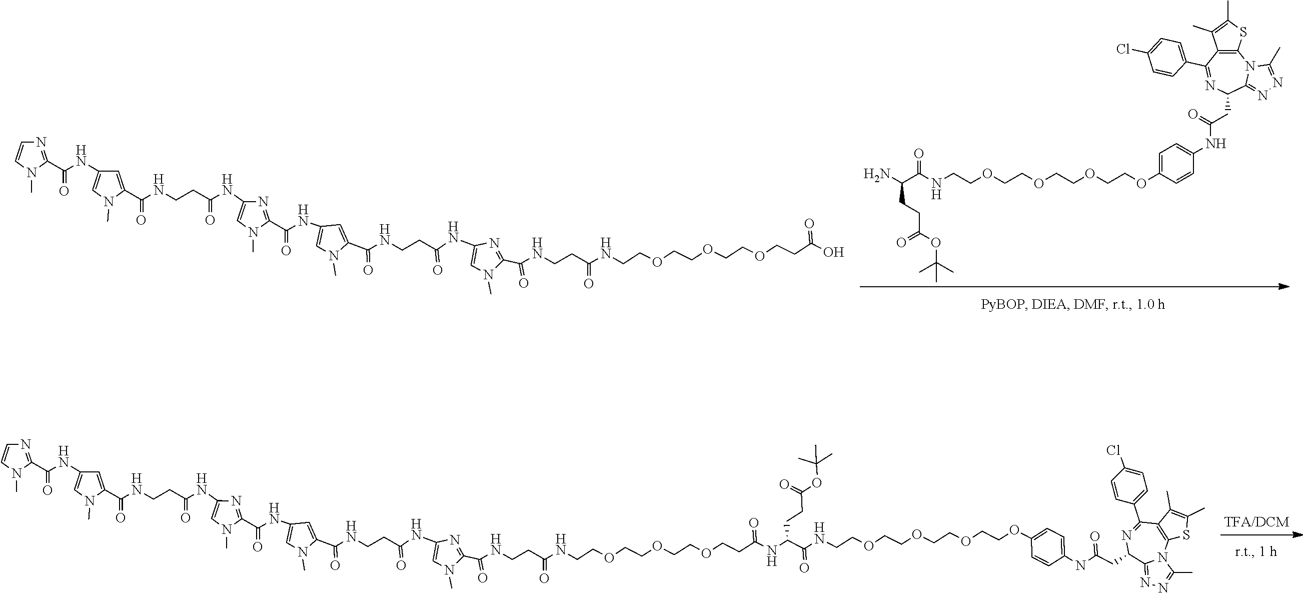

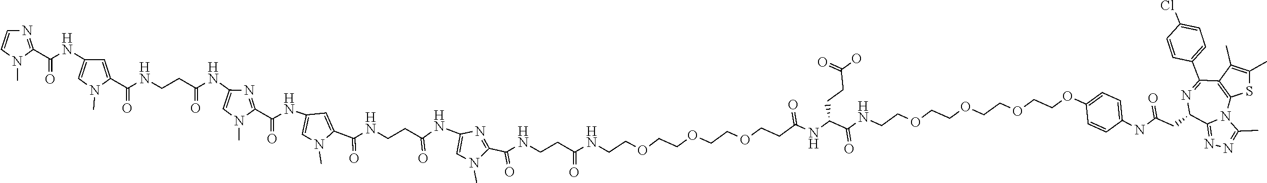

### Step 1: Synthesis of tert-butyl (S)-(2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 150.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 200.00 mg of tert-butyl (S)-(2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamate was obtained as yellow oil (85.49% yield). LC/MS: mass calcd. For C₃₈H₄₇ClN₆O₇S: 766.29, found: 767.50 [M+H].

### Step 2: Synthesis of (S)—N-(4-(2-(2-(2-(2-aminoethoxy)ethoxy)ethoxy)ethoxy)phenyl)-2-(4-(4-chlorophenyl)-2,3,9-trimethyl-6H-thieno[3,2-f][1,2,4]triazolo[4,3-a][1,4]diazepin-6-yl)acetamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 200.00 mg of tert-butyl(S)-(2-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamate was used, 300.00 mg crude of (S)—N-(4-(2-(2-(2-(2-aminoethoxy)ethoxy)ethoxy)ethoxy)phenyl)-2-(4-(4-chlorophenyl)-2,3,9-trimethyl-6H-thieno[3,2-f][1,2,4]triazolo[4,3-a][1,4]diazepin-6-yl)acetamide was obtained as yellow oil. LC/MS: mass calcd. For C₃₃H₃₉ClN₆O₅S: 666.24, found: 667.45 [M+H]⁺.

### Step 3: Synthesis of tert-butyl (R)-14-((((9H-fluoren-9-yl)methoxy)carbonylamino)-1-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-13-oxo-3,6,9-trioxa-12-azaheptadecan-17-oate

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide, but this product was purified by reverse phase column. 150.00 mg crude of (S)—N-(4-(2-(2-(2-(2-aminoethoxy)ethoxy)ethoxy)ethoxy)phenyl)-2-(4-(4-chlorophenyl)-2,3,9-trimethyl-6H-thieno[3,2-f][1,2,4]triazolo[4,3-a][1,4]diazepin-6-yl)acetamide was used, 100.00 mg of tert-butyl (R)-14-((((9H-fluoren-9-yl)methoxy)carbonyl)amino)-1-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-13-oxo-3,6,9-trioxa-12-azaheptadecan-17-oate was obtained as yellow oil (37.25% yield). LC/MS: mass calcd. For C₅₇H₆₄ClN₇O₁₀S: 1073.41, found: 1074.55 [M+H]⁺.

### Step 4: Synthesis of tert-butyl (R)-14-amino-1-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-13-oxo-3,6,9-trioxa-12-azaheptadecan-17-oate

Into a 25 ml flask was added tert-butyl (R)-14-((((9H-fluoren-9-yl)methoxy) carbonyl)amino)-1-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-13-oxo-3,6,9-trioxa-12-azaheptadecan-17-oate (100.00 mg, 0.09 mmol, 1.00 equiv), piperidine (0.50 mL), DMF (2.50 mL), the reaction was stirred at room temperature for 2 h. The reaction mixture was purified by reverse flash chromatography with the following conditions: column, C18 column; mobile phase, ACN in water (0.05% TFA), 20% to 50% gradient in 50 min; detector, UV 254 nm. The fractions were combined and concentrated. This resulted in tert-butyl (R)-14-amino-1-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-13-oxo-3,6,9-trioxa-12-azaheptadecan-17-oate (50.00 mg, 58.86%) as yellow oil. LC/MS: mass calcd. For C₄₂H₅₄ClN₇O₈S: 851.34, found: 852.60 [M+H]⁺.

### Step 5: Synthesis of tert-butyl 3-[2-(2-[2-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]ethoxy]ethoxy)ethoxy]propanoate

The procedure was the same as (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide, but this product was purified by reverse phase column. 300.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl] formamido)propanamido]imidazol-2-yl]formamido)propanoic acid was used, 386.00 mg of tert-butyl 3-[2-(2-[2-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]ethoxy]ethoxy)ethoxy]propanoate was obtained as yellow oil (96.36%). LC/MS: mass calcd. For C₄₉H₆₈N₁₆O₁₃: 1088.52, found: 1111.70 [M+Na]⁺.

### Step 6: Synthesis of 3-[2-(2-[2-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]ethoxy]ethoxy)ethoxy]propanoic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 386.00 mg of tert-butyl 3-[2-(2-[2-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]ethoxy]ethoxy)ethoxy]propanoate was used, 400.00 mg crude of 3-[2-(2-[2-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]ethoxy]ethoxy)ethoxy]propanoic acid was obtained as yellow oil. LC/MS: mass calcd. For C₄₅H₆₀N₁₆O₁₃: 1032.45, found: 1033.65[M+H]⁺.

### Step 7: Synthesis of tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate

Into a 8 mL flask was added 3-[2-(2-[2-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]ethoxy]ethoxy)ethoxy]propanoic acid (43.00 mg, 0.04 mmol, 1.00 equiv), DMF (2.50 mL), tert-butyl (R)-14-amino-1-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-13-oxo-3,6,9-trioxa-12-azaheptadecan-17-oate (35.00 mg, 0.04 mmol, 1.00 equiv), PyBOP (22.00 mg, 0.04 mmol, 1.00 equiv), the mixture was stirred at room temperature for 5 mins, then DIEA (22.00 mg, 0.17 mmol, 4.15 equiv) was added, the reaction was stirred at room temperature for 1 h. The reaction mixture was purified by reverse flash chromatography with the following conditions: C18 column; mobile phase A: 0.05% TFA in water, mobile phase B: MeCN, 10% to 50% gradient in 50 min; detector, UV 254 nm. The fractions were combined and concentrated. This resulted in tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate (60.00 mg, 72.68%) as yellow oil. LC/MS: mass calcd. For C₈₇H₁₁₂ClN₂₃O₂₀S: 1865.79, found: 934.70 [M/2+H]⁺.

### Step 8: Synthesis of (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oic acid

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. But the product was purified by Prep-HPLC. 55.00 mg of tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate was used, 9.40 mg of (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oic acid was obtained as white solid (17.08% yield). HRMS: mass calcd. For C₈₃H₁₀₄ClN₂₃O₂₀S: 1809.7237, found: 1810.7229 [M+H]⁺.

### (20R,23R)-20-(2-carboxyethyl)-23-((2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18,21-tetraoxo-9,12,15-trioxa-2,6,19,22-tetraazahexacosan-26-oic acid (Compound 69)

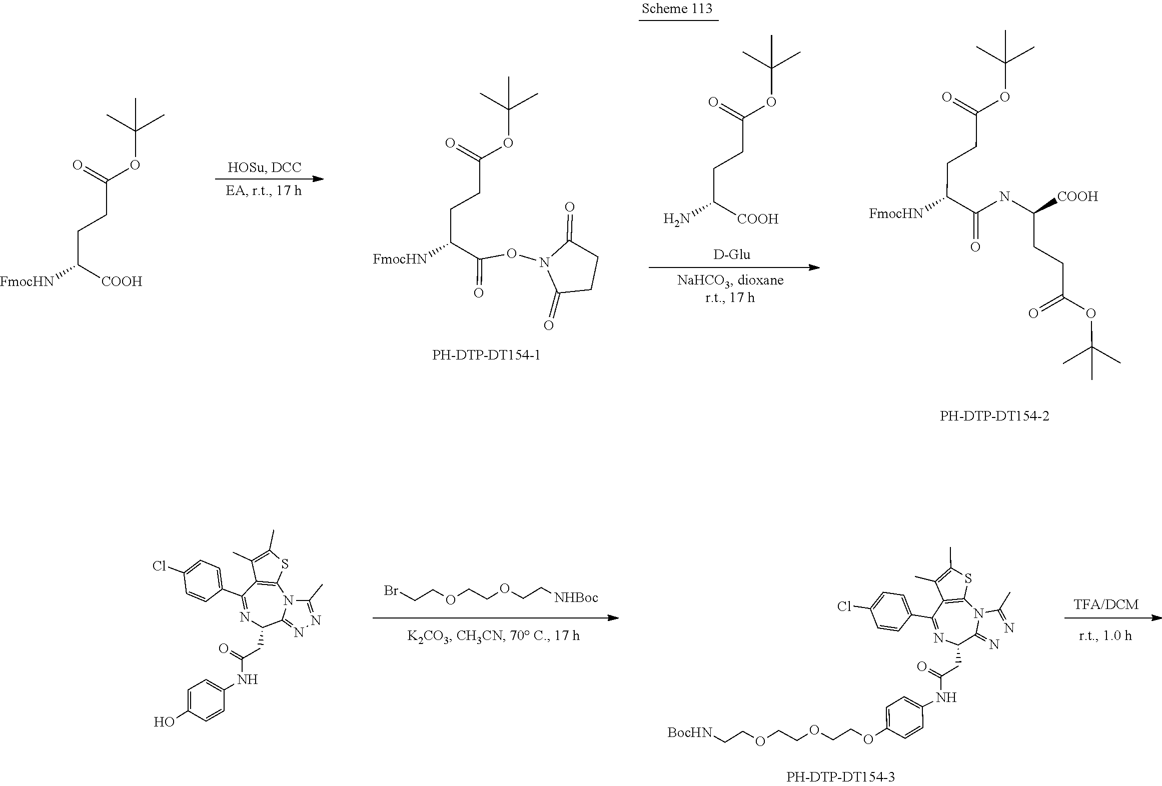

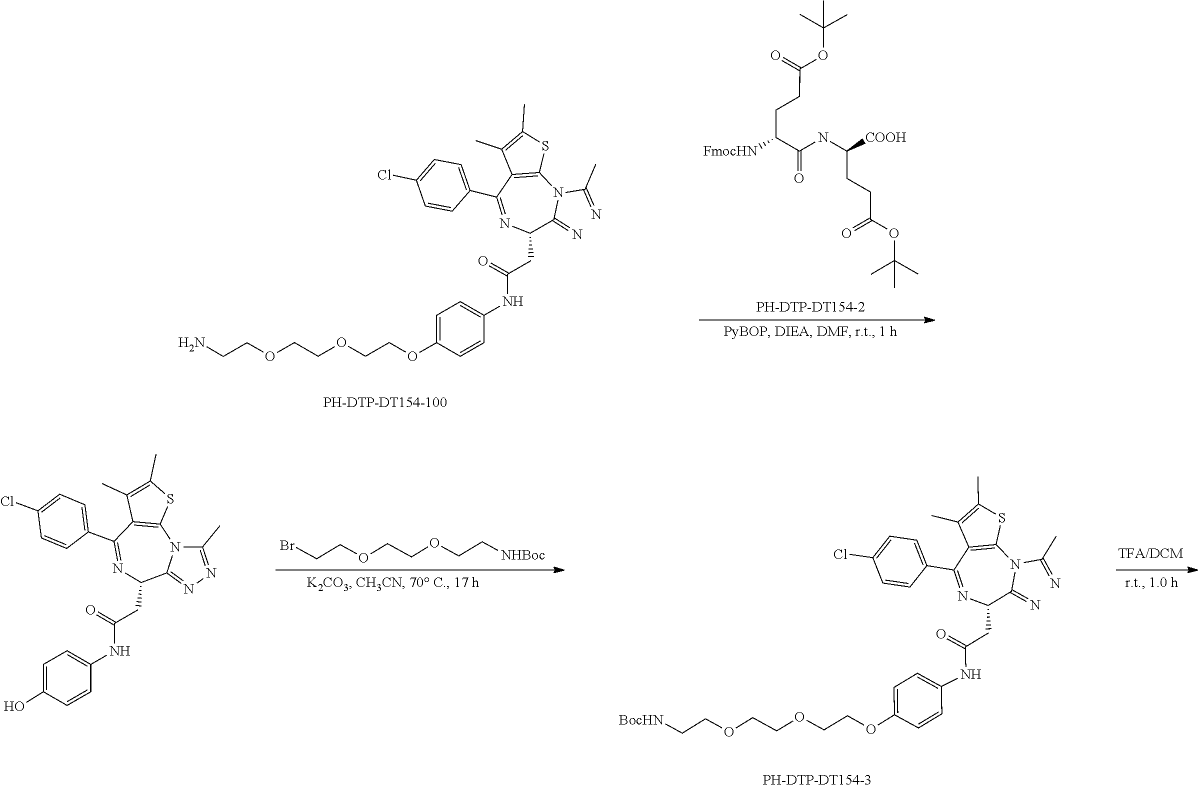

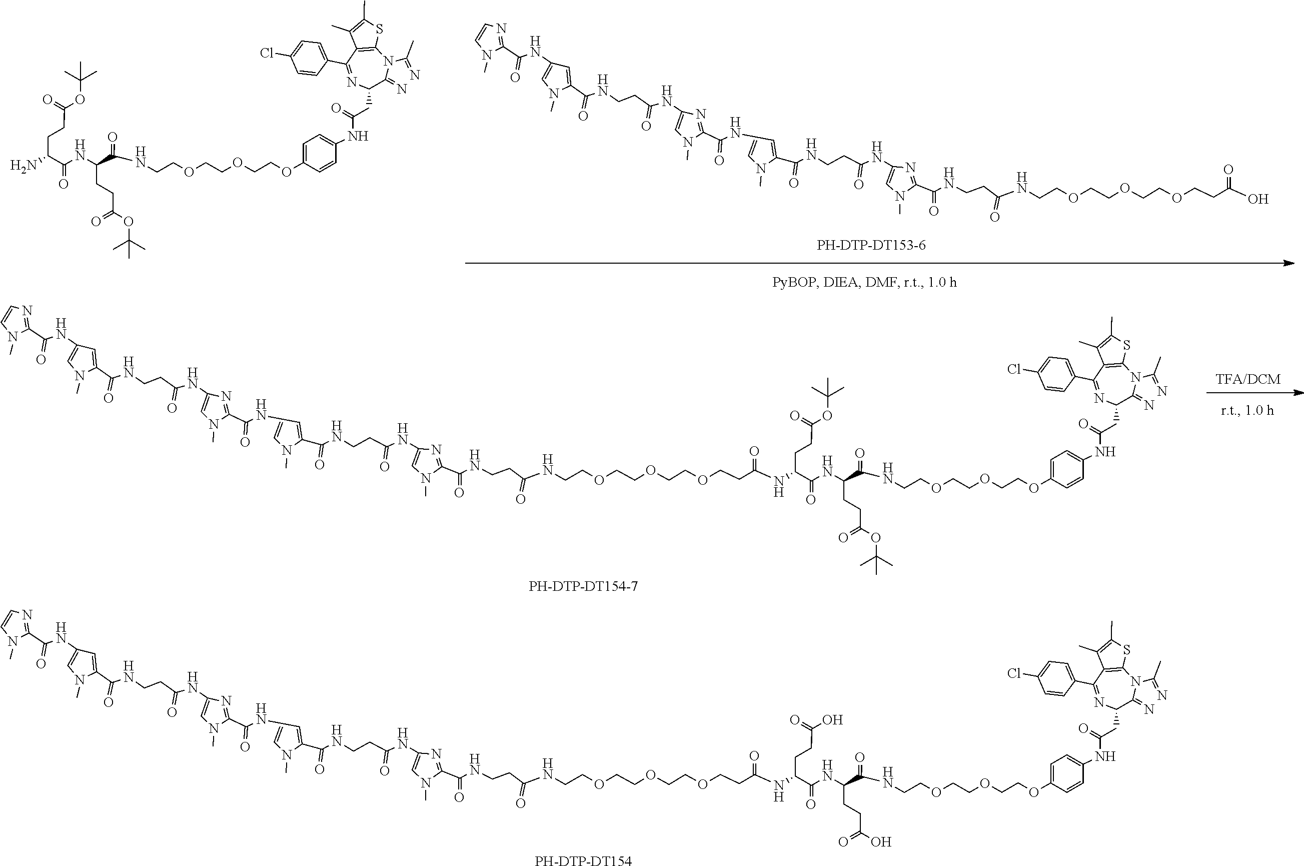

### Step 1: Synthesis of 1-tert-butyl 2,5-dioxopyrrolidin-1-yl (4R)-4-[[(9H-fluoren-9-ylmethoxy)carbonyl]amino]pentanedioate

Into a 100 mL flask was added (2R)-5-(tert-butoxy)-2-[[(9H-fluoren-9-ylmethoxy)carbonyl]amino]-5-oxopentanoic acid (1.00 g, 2.35 mmol, 1.00 equiv), EA (15.00 mL), HOSu (0.41 g, 3.53 mmol, 1.50 equiv), DCC (0.58 g, 2.82 mmol, 1.20 equiv), the reaction was stirred at room temperature for 17 h.

The resulting mixture was filtered, the filter cake was washed with EA (3×10 mL). The filtrate was concentrated under reduced pressure. This resulted in 1-tert-butyl 2,5-dioxopyrrolidin-1-yl(4R)-4-[[(9H-fluoren-9-ylmethoxy)carbonyl]amino]pentanedioate (1.25 g, 95.39%) as white solid. LC/MS: mass calcd. For C₂₈H₃₀N₂O₈: 522.20, found: 545.35 [M+Na]⁺.

### Step 2: Synthesis of (2R)-5-(tert-butoxy)-2-[(2R)-5-(tert-butoxy)-2-[[(9H-fluoren-9-ylmethoxy)carbonyl]amino]-5-oxopentanamido]-5-oxopentanoic acid

Into a 100 mL flask was added 1-tert-butyl 2,5-dioxopyrrolidin-1-yl (4R)-4-[[(9H-fluoren-9-ylmethoxy)carbonyl]amino]pentanedioate (1.25 g, 2.39 mmol, 1.00 equiv), dioxane (15.00 mL), the mixture was stirred at room temperature, then (2R)-2-amino-5-(tert-butoxy)-5-oxopentanoic acid (0.73 g, 3.59 mmol, 1.50 equiv) and the solution of NaHCO₃ (0.40 g, 4.78 mmol, 2.00 equiv) in H₂O (20.00 mL) were added to the above solution, the reaction was stirred at room temperature for 17 h. The reaction was cooled to 0 degrees C., adjust to pH=4-5 by 2M HCl, then extracted with EA (3×20 mL), the organic phase was washed with water (30 mL), NaCl solution (30 mL), dried over anhydrous Na₂SO₄. The solid was filtered out and the filtrate was concentrated. This resulted in (2R)-5-(tert-butoxy)-2-[(2R)-5-(tert-butoxy)-2-[[(9H-fluoren-9-ylmethoxy)carbonyl]amino]-5-oxopentanamido]-5-oxopentanoic acid (1.20 g, 71.47%) as white oil. LC/MS: mass calcd. For C₃₃H₄₂N₂O₉: 610.29, found: 611.30 [M+H]⁺.

### Step 3: Synthesis of tert-butyl (S)-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethyl)carbamate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 200.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 240.00 mg of tert-butyl (S)-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethyl)carbamate was obtained as yellow oil (81.05% yield). LC/MS: mass calcd. For C₃₆H₄₃ClN₆O₆S: 722.27, found: 723.45 [M+H]⁺.

### Step 3: Synthesis of (S)—N-(4-(2-(2-(2-aminoethoxy)ethoxy)ethoxy)phenyl)-2-(4-(4-chlorophenyl)-2,3,9-trimethyl-6H-thieno[3,2-f][1,2,4]triazolo[4,3-a][1,4]diazepin-6-yl)acetamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 120.00 mg of tert-butyl (S)-(2-(2-(2-(4-(2-(4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethyl)carbamate was used, 120.00 mg crude of (S)—N-(4-(2-(2-(2-aminoethoxy)ethoxy)ethoxy)phenyl)-2-(4-(4-chlorophenyl)-2,3,9-trimethyl-6H-thieno[3,2-f][1,2,4]triazolo[4,3-a][1,4]diazepin-6-yl)acetamide was obtained as yellow oil. LC/MS: mass calcd. For C₃₁H₃₅ClN₆O₄S: 622.21, found: 623.35 [M+H]⁺.

### Step 4: Synthesis of tert-butyl (11R,14R)-14-((((9H-fluoren-9-yl)methoxy)carbonyl)amino)-11-(3-(tert-butoxy)-3-oxopropyl)-1-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-10,13-dioxo-3,6-dioxa-9,12-diazaheptadecan-17-oate

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate. 98.00 mg of (2R)-5-(tert-butoxy)-2-[(2R)-5-(tert-butoxy)-2-[[(9H-fluoren-9-ylmethoxy)carbonyl]amino]-5-oxopentanamido]-5-oxopentanoic acid was used, 145.00 mg of tert-butyl (11R,14R)-14-((((9H-fluoren-9-yl)methoxy)carbonyl)amino)-11-(3-(tert-butoxy)-3-oxopropyl)-1-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-10,13-dioxo-3,6-dioxa-9,12-diazaheptadecan-17-oate was obtained as yellow oil (72.23% yield). LC/MS: mass calcd. For C₆₄H₇₅ClN₈O₁₂S: 1214.49, found: 1215.50 [M+H]⁺.

### Step 5: Synthesis of tert-butyl (11R,14R)-14-amino-11-(3-(tert-butoxy)-3-oxopropyl)-1-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-10,13-dioxo-3,6-dioxa-9,12-diazaheptadecan-17-oate

Into a 25 mL flask was added tert-butyl (11R,14R)-14-((((9H-fluoren-9-yl)methoxy)carbonyl)amino)-11-(3-(tert-butoxy)-3-oxopropyl)-1-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-10,13-dioxo-3,6-dioxa-9,12-diazaheptadecan-17-oate (140.00 mg, 0.12 mmol, 1.00 equiv), DCM (3.00 mL), diethylamine (84.0 mg, 1.15 mmol, 10.00 equiv), the reaction was stirred at 40 degrees C. for 1 h. The reaction was concentrated. The residue was purified by TLC Plate with DCM:MeOH=10:1. This resulted in tert-butyl (11R,14R)-14-amino-11-(3-(tert-butoxy)-3-oxopropyl)-1-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-10,13-dioxo-3,6-dioxa-9,12-diazaheptadecan-17-oate (60.00 mg, 47.20%) as yellow oil. LC/MS: mass calcd. For C₄₉H₆₅ClN₈O₁₀S: 992.42, found: 993.70 [M+H]⁺.

### Step 6: Synthesis of tert-butyl (20R,23R)-20-(3-(tert-butoxy)-3-oxopropyl)-23-((2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18,21-tetraoxo-9,12,15-trioxa-2,6,19,22-tetraazahexacosan-26-oate

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate. 55.00 mg of 3-[2-(2-[2-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido) propanamido]imidazol-2-yl]formamido)propanamido]ethoxy]ethoxy)ethoxy]propanoic acid was used, 100.00 mg of tert-butyl (20R,23R)-20-(3-(tert-butoxy)-3-oxopropyl)-23-((2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18,21-tetraoxo-9,12,15-trioxa-2,6,19,22-tetraazahexacosan-26-oate was obtained as yellow oil (65.14% yield). LC/MS: mass calcd. For C₉₄H₁₂₃ClN₂₄O₂₂S: 2006.87, found: 1005.45 [M/2+H]⁺.

### Step 7: Synthesis of (20R,23R)-20-(2-carboxyethyl)-23-((2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18,21-tetraoxo-9,12,15-trioxa-2,6,19,22-tetraazahexacosan-26-oic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid, but the product was purified by Prep-HPLC. 100.00 mg of tert-butyl (20R,23R)-20-(3-(tert-butoxy)-3-oxopropyl)-23-((2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18,21-tetraoxo-9,12,15-trioxa-2,6,19,22-tetraazahexacosan-26-oate was used, 12.70 mg of (20R,23R)-20-(2-carboxyethyl)-23-((2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18,21-tetraoxo-9,12,15-trioxa-2,6,19,22-tetraazahexacosan-26-oic acid was obtained as white solid (12.06% yield). HRMS: mass calcd. For C₈₆H₁₀₇ClN₂₄O₂₂S: 1894.7401, found: 1895.7407 [M+H]⁺.

### N-(5-((3-((2-(((11S,14S)-1-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-11,14-bis(3-guanidinopropyl)-10,13,16,29-tetraoxo-3,6,19,22,25-pentaoxa-9,12,15,28-tetraazahentriacontan-31-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 99)

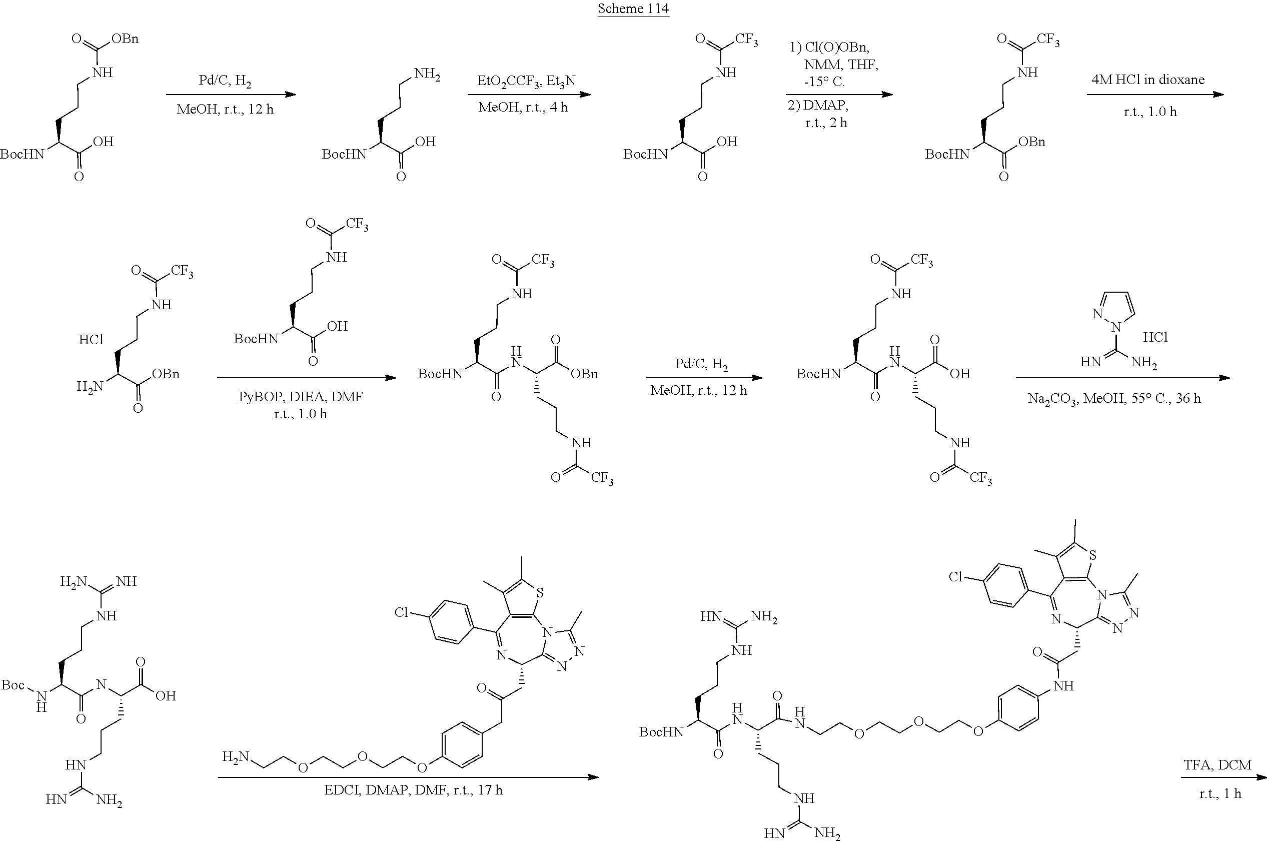

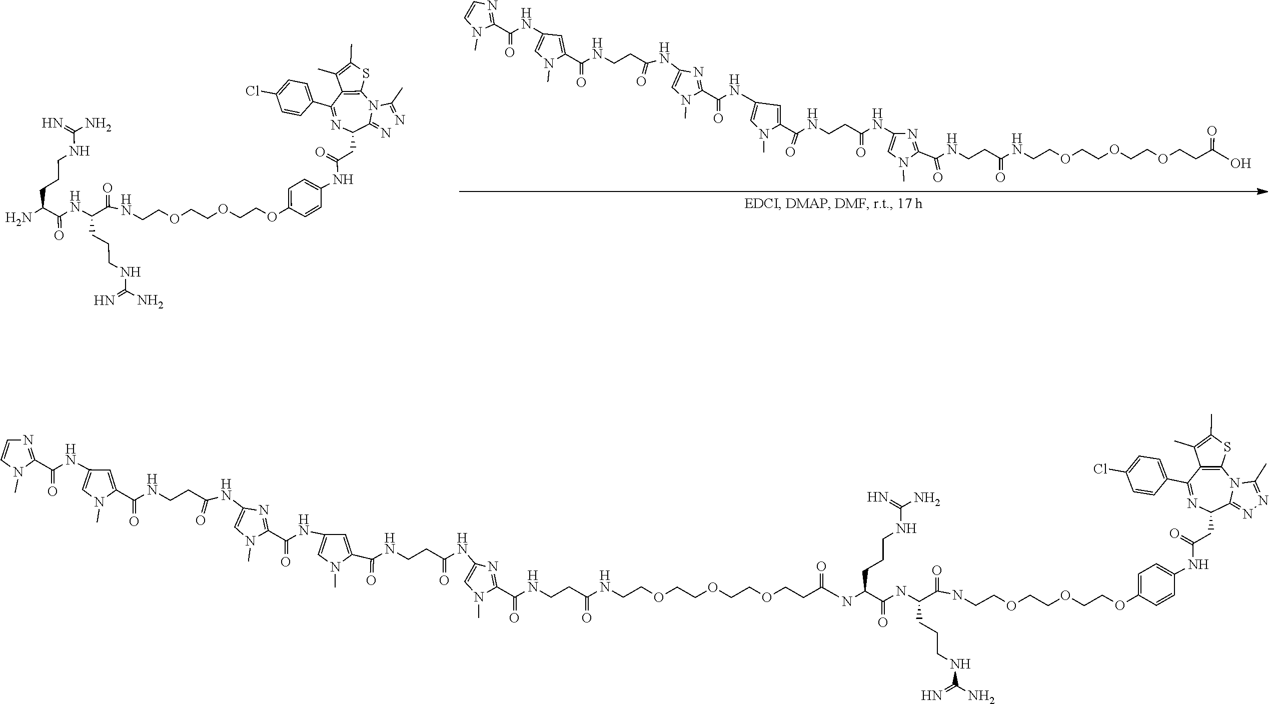

### Step 1: Synthesis of (2S)-5-amino-2-[(tert-butoxycarbonyl)amino]pentanoic acid

To a stirred solution of (2S)-5-[[(benzyloxy)carbonyl]amino]-2-[(tert-butoxycarbonyl)amino]pentanoic acid (10.00 g, 27.29 mmol, 1.00 equiv) in MeOH (150.00 mL) was added Pd/C (1.0 g, 10% w/w) at room temperature. The resulting mixture was stirred for 17 h at room temperature under H₂ atmosphere. Then the resulting mixture was filtered with diatomite, the organic layers was concentrated under vacuum. (2S)-5-amino-2-[(tert-butoxycarbonyl)amino]pentanoic acid (10.00 g, crude) was obtained as colorless oil. LC/MS: mass calcd. For C₁₀H₂₀N₂O₄: 232.14, found: 233.10 [M+H]⁺.

### Step 2: Synthesis of (2S)-2-[(tert-butoxycarbonyl)amino]-5-(2,2,2-trifluoroacetamido)pentanoic acid

To a stirred solution of (2S)-5-amino-2-[(tert-butoxycarbonyl)amino]pentanoic acid (10.00 g, 43.05 mmol, 1.00 equiv) in MeOH (300.00 mL) was added trifluoroethyl acetate (9.17 g, 64.58 mmol, 1.50 equiv) and Et₃N (8.71 g, 86.10 mmol, 2.00 equiv) at room temperature. The resulting mixture was stirred for 4 h at room temperature. The resulting mixture was concentrated under vacuum. (2S)-2-[(tert-butoxycarbonyl)amino]-5-(2,2,2-trifluoroacetamido)pentanoic acid (9.40 g, 66.51%) was obtained as yellow oil. LC/MS: mass calcd. For C₁₂H₁₉F₃N₂O₅: 328.12, found: 351.05 [M+Na]⁺.

### Step 3: Synthesis of benzyl (2S)-2-[(tert-butoxycarbonyl)amino]-5-(2,2,2-trifluoroacetamido)pentanoate

To a stirred solution of (2S)-2-[(tert-butoxycarbonyl)amino]-5-(2,2,2-trifluoroacetamido)pentanoic acid (5.00 g, 15.23 mmol, 1.00 equiv) in THF (40.00 mL) was added NMM (1.54 g, 15.23 mmol, 1.00 equiv) dropwise at room temperature. The result mixture was cooled to −15 degrees C. and benzyl chloroformate (2.73 g, 15.99 mmol, 1.05 equiv) in THF (10.00 mL) was added to the result mixture at −15 degrees C. The resulting mixture was stirred for 2 min at −15 degrees C. and warmed to 0 degrees C. and stirred for 15 mins. DMAP (0.47 g, 3.81 mmol, 0.25 equiv) was added to the result mixture and the result mixture was allowed to warm to room temperature and stirred for 2 h. The reaction was quenched with H₂O (50 mL) at 0 degrees C. The resulting mixture was extracted with EA (3×100 mL). The combined organic layers were washed with brine (1×100 mL), dried over anhydrous Na₂SO₄. After filtration, the filtrate was concentrated under reduced pressure. Benzyl (2S)-2-[(tert-butoxycarbonyl)amino]-5-(2,2,2-trifluoroacetamido)pentanoate (6.00 g, 94.15%) was obtained as yellow oil. LC/MS: mass calcd. For C₁₉H₂₅F₃N₂O₅: 418.17, found: 441.25 [M+Na]⁺.

### Step 4: Synthesis of benzyl (2S)-2-amino-5-(2,2,2-trifluoroacetamido)pentanoate hydrochloride

A solution of benzyl (2S)-2-[(tert-butoxycarbonyl)amino]-5-(2,2,2-trifluoroacetamido)pentanoate (6.00 g, 14.34 mmol, 1.00 equiv) in HCl/1,4-dioxane (4M, 60.00 mL) was stirred for 1 h at room temperature. The resulting mixture was concentrated under reduced pressure. Benzyl (2S)-2-amino-5-(2,2,2-trifluoroacetamido)pentanoate hydrochloride (5.00 g, crude) was obtained as yellow solid. LC/MS: mass calcd. For C₁₄H₁₈ClF₃N₂O₃: 318.12, found: 319.10 [M+H]⁺.

### Step 5: Synthesis of benzyl (2S)-2-[(2S)-2-[(tert-butoxycarbonyl)amino]-5-(2,2,2-trifluoroacetamido)pentanamido]-5-(2,2,2-trifluoroacetamido)pentanoate

To a stirred solution of (2S)-2-[(tert-butoxycarbonyl)amino]-5-(2,2,2-trifluoroacetamido)pentanoic acid (4.63 g, 14.10 mmol, 1.00 equiv) in DMF (60.00 mL) was added PyBO P (11.00 g, 21.14 mmol, 1.50 equiv), benzyl (2S)-2-amino-5-(2,2,2-trifluoroacetamido)pentanoate hydrochloride (5.00 g, 14.09 mmol, 1.00 equiv) and DIEA (5.46 g, 42.28 m mol, 3.00 equiv) in portions at 0 degrees C. The resulting mixture was stirred for 1 h at room temperature. The reaction was quenched with Water/Ice (180 mL) at 0 degrees C. The precipitated solids were collected by filtration and washed with H₂O (3×20 mL), dried under vacuum. Benzyl (2S)-2-[(2S)-2-[(tert-butoxycarbonyl)amino]-5-(2,2,2-trifluoroacetamido)pentanamido]-5-(2,2,2-trifluoroacetamido)pentanoate (5.00 g, 56.44%) was obtained as light yellow solid. LC/MS: mass calcd. For C₂₆H₃₄F₆N₄O₇: 628.23, found: 646.15 [M+H₂O]⁺.

### Step 6: Synthesis of (2S)-2-[(2S)-2-[(tert-butoxycarbonyl)amino]-5-(2,2,2-trifluoroacetamido)pentanamido]-5-(2,2,2-trifluoroacetamido)pentanoic acid

The procedure was the same as (2S)-5-amino-2-[(tert-butoxycarbonyl)amino]pentanoic acid. 2.00 g of benzyl (2S)-2-[(2S)-2-[(tert-butoxycarbonyl)amino]-5-(2,2,2-trifluoroacetamido)pentanamido]-5-(2,2,2-trifluoroacetamido)pentanoate was used, 1.30 g of (2S)-2-[(2S)-2-[(tert-butoxycarbonyl)amino]-5-(2,2,2-trifluoroacetamido)pentanamido]-5-(2,2,2-trifluoroacetamido)pentanoic acid was obtained as white solid (75.88% yield). LC/MS: mass calcd. For C₁₉H₂₈F₆N₄O₇: 538.19, found: 539.20 [M+H]⁺.

### Step 7: Synthesis of (2S)-2-[(2S)-2-[(tert-butoxycarbonyl)amino]-5-carbamimidamidopentanamido]-5-carbamimidamidopentanoic acid

To a stirred solution of (2S)-2-[(2S)-2-[(tert-butoxycarbonyl)amino]-5-(2,2,2-trifluoroacetamido)pentanamido]-5-(2,2,2-trifluoroacetamido)pentanoic acid (800.00 mg, 1.49 mmol, 1.00 equiv) in MeOH (20.00 mL) and sat. Na₂CO₃ aqueous (10.00 mL) was added pyrazole-1-carboximidamide hydrochloride (872.00 mg, 5.96 mmol, 4.00 equiv) in portions at room temperature. The resulting mixture was stirred for 36 h at 55 degrees C. The resulting mixture was filtered, the filter cake was washed with MeOH (3×10 mL). The filtrate was concentrated under reduced pressure. The residue was purified by reverse flash chromatography with the following conditions: column, C18 silica gel; mobile phase, ACN in water (0.05% NH₄HCO₃), 0% to 10% gradient in 30 min; detector, UV 254 nm. The fractions were combined and concentrated. (2S)-2-[(2S)-2-[(tert-butoxycarbonyl)amino]-5-carbamimidamidopentanamido]-5-carbamimidamidopentanoic acid (400.00 mg, 62.54%) was obtained as white solid. LC/MS: mass calcd. For C₁₇H₃₄N₈O₅: 430.27, found: 431.25 [M+H]⁺.

### Step 8: Synthesis of tert-butyl ((6S,9S)-1-amino-19-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-9-(3-guanidinopropyl)-1-imino-7,10-dioxo-14,17-dioxa-2,8,11-triazanonadecan-6-yl)carbamate

The procedure was the same as N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide. The product was purified by reverse phase column. 90.00 mg of (2S)-2-[(2S)-2-[(tert-butoxycarbonyl)amino]-5-carbamimidamidopentanamido]-5-carbamimidamidopentanoic acid was used, 100.00 mg of tert-butyl ((6S,9S)-1-amino-19-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-9-(3-guanidinopropyl)-1-imino-7,10-dioxo-14,17-dioxa-2,8,11-triazanonadecan-6-yl)carbamate was obtained as yellow oil (46.19% yield). LC/MS: mass calcd. For C₄₈H₆₇ClN₁₄O₈S: 1034.47, found: 1035.55 [M+H]⁺.

### Step 9: Synthesis of (S)-2-amino-N—((S)-1-amino-16-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-1-imino-7-oxo-11,14-dioxa-2,8-diazahexadecan-6-yl)-5-guanidinopentanamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 100.00 mg of tert-butyl ((6S,9S)-1-amino-19-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-9-(3-guanidinopropyl)-1-imino-7,10-dioxo-14,17-dioxa-2,8,11-triazanonadecan-6-yl)carbamate was used, 100.00 mg crude of (S)-2-amino-N—((S)-1-amino-16-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-1-imino-7-oxo-11,14-dioxa-2,8-diazahexadecan-6-yl)-5-guanidinopentanamide was obtained as yellow oil. LC/MS: mass calcd. For C₄₃H₅₉ClN₁₄O₆S: 934.42, found: 935.65 [M+H]⁺.

### Step 10: Synthesis of N-(5-((3-((2-(((11S,14S)-1-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)-11,14-bis(3-guanidinopropyl)-10,13,16,29-tetraoxo-3,6,19,22,25-pentaoxa-9,12,15,28-tetraazahentriacontan-31-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide. 100.00 mg of 3-[2-(2-[2-[3-([1-methyl-4-(3-([[1-methyl-4-(1-methyl-4-[[3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]ethoxy]ethoxy)ethoxypropanoic acid was used, 10.00 mg of N-(5-(3-((2-(((11S,14S)-1-(4-(2-((S)-4(4-chlorophenyl)-2,3,9-trimethyl-6H-thieno[3,2-f][1,2,4]triazolo(4,3-a][1,4]diazepin-6-yl)acetamido)phenoxy)-11,14-bis(3-guanidinopropyl)-10,13,16,29-tetraoxo-3,6,19,22,23-pentaoxa-9,12,15,28-tetraazahentriacontan-31-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as yellow solid (5.17% yield). HRMS: mass calcd. For C₈₈H₁₁₇ClN₃₀O₁₈S: 1948.8571, found: 1949.8616 [M+H]⁺.

### N-(5-((3-((2-(((11S,14S)-1-(4-(2-((S)-6-(4-chlorophenyl)-8-methoxy-1-methyl-4H-benzo[f][1,2,4]triazolo[4,3-a][1,4]diazepin-4-yl)acetamido)phenoxy)-11,14-bis(3-guanidinopropyl)-10,13,16,29-tetraoxo-3,6,19,22,25-pentaoxa-9,12,15,28-tetraazahentriacontan-31-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 100)

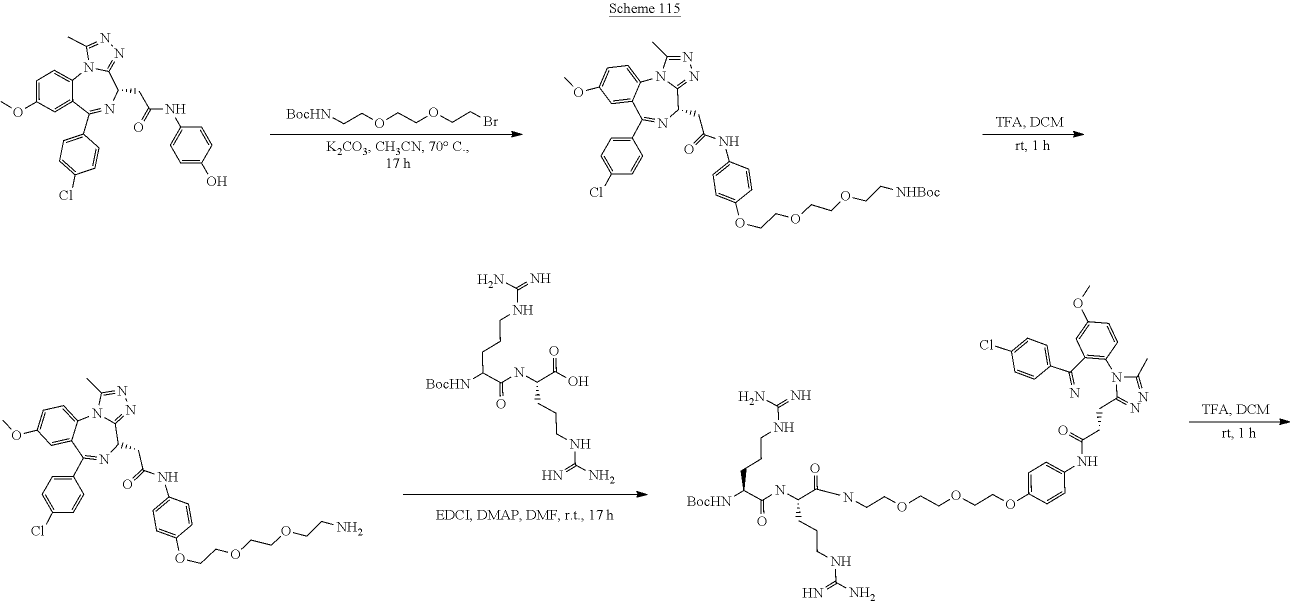

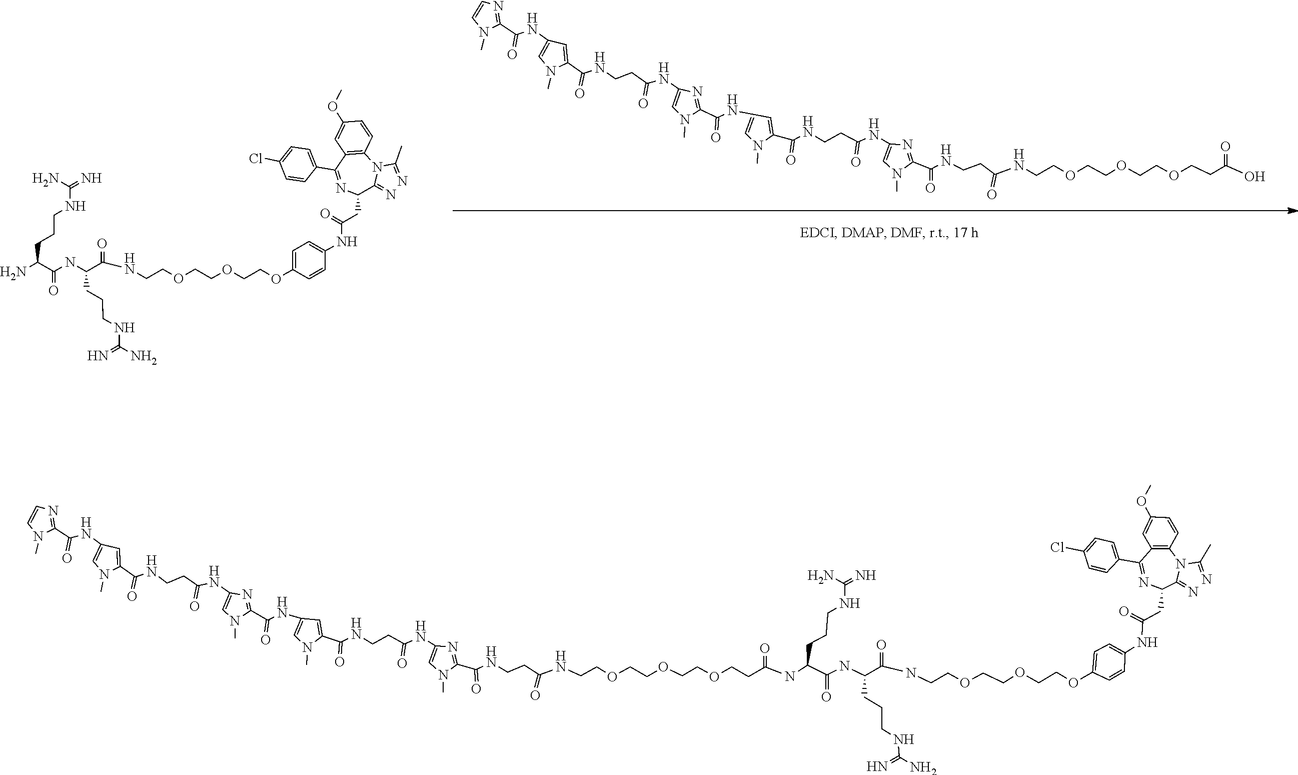

### Step 1: Synthesis of tert-butyl (S)-(2-(2-(2-(4-(2-(6-(4-chlorophenyl)-8-methoxy-1-methyl-4H-benzo[f][1,2,4]triazolo[4,3-a][1,4]diazepin-4-yl)acetamido)phenoxy)ethoxy)ethoxy)ethyl)carbamate

The procedure was the same as tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate. 100.00 mg of (S)-2-(6-(4-chlorophenyl)-8-methoxy-1-methyl-4H-benzo[f][1,2,4]triazolo[4,3-a][1,4]diazepin-4-yl)-N-(4-hydroxyphenyl)acetamide was used, 65.00 mg of tert-butyl (S)-(2-(2-(2-(4-(2-(6-(4-chlorophenyl)-8-methoxy-1-methyl-4H-benzo[f][1,2,4]triazolo[4,3-a][1,4]diazepin-4-yl)acetamido)phenoxy)ethoxy)ethoxy)ethyl)carbamate was obtained as yellow oil (44.10% yield). LC/MS: mass calcd. For C₃₇H₄₃ClN₆O₇: 718.29, found: 719.30 [M+H]⁺.

### Step 2: Synthesis of (S)—N-(4-(2-(2-(2-aminoethoxy)ethoxy)ethoxy)phenyl)-2-(6-(4-chlorophenyl)-8-methoxy-1-methyl-4H-benzo[f][1,2,4]triazolo[4,3-a][1,4]diazepin-4-yl)acetamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 65.00 mg of tert-butyl (S)-(2-(2-(2-(4-(2-(6-(4-chlorophenyl)-8-methoxy-1-methyl-4H-benzo[f][1,2,4]triazolo[4,3-a][1,4]diazepin-4-yl)acetamido)phenoxy)ethoxy)ethoxy)ethyl)carbamate was used, 65.00 mg crude of (S)—N-(4-(2-(2-(2-aminoethoxy)ethoxy)ethoxy)phenyl)-2-(6-(4-chlorophenyl)-8-methoxy-1-methyl-4H-benzo[f][1,2,4]triazolo[4,3-a][1,4]diazepin-4-yl)acetamide was obtained as yellow oil. LC/MS: mass calcd. For C₃₂H₃₅ClN₆O₅: 618.24, found: 619.45 [M+H]⁺.

### Step 3: Synthesis of tert-butyl ((6S,9S)-1-amino-19-(4-(2-((S)-6-(4-chlorophenyl)-8-methoxy-1-methyl-4H-benzo[f][1,2,4]triazolo[4,3-a][1,4]diazepin-4-yl)acetamido)phenoxy)-9-(3-guanidinopropyl)-1-imino-7,10-dioxo-14,17-dioxa-2,8,11-triazanonadecan-6-yl)carbamate

The procedure was the same as N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide. The product was purified by reverse phase column. 60.00 mg of (S)—N-(4-(2-(2-(2-aminoethoxy)ethoxy)ethoxy)phenyl)-2-(6-(4-chlorophenyl)-8-methoxy-1-methyl-4H-benzo[f][1,2,4]triazolo[4,3-a][1,4]diazepin-4-yl)acetamide was used, 60.00 mg of tert-butyl ((6S,9S)-1-amino-19-(4-(2-((S)-6-(4-chlorophenyl)-8-methoxy-1-methyl-4H-benzo[f][1,2,4]triazolo[4,3-a][1,4]diazepin-4-yl)acetamido)phenoxy)-9-(3-guanidinopropyl)-1-imino-7,10-dioxo-14,17-dioxa-2,8,11-triazanonadecan-6-yl)carbamate was obtained as yellow oil (54.03% yield). LC/MS: mass calcd. For C₄₉H₆₇ClN₁₄O₉: 1030.49, found: 516.45 [M/2+H]⁺.

### Step 4: Synthesis of (S)-2-amino-N—((S)-1-amino-16-(4-(2-((S)-6-(4-chlorophenyl)-8-methoxy-1-methyl-4H-benzo[f][1,2,4]triazolo[4,3-a][1,4]diazepin-4-yl)acetamido)phenoxy)-1-imino-7-oxo-11,14-dioxa-2,8-diazahexadecan-6-yl)-5-guanidinopentanamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 40.00 mg of tert-butyl ((6S,9S)-1-amino-19-(4-(2-((S)-6-(4-chlorophenyl)-8-methoxy-1-methyl-4H-benzo[f][1,2,4]triazolo[4,3-a][1,4]diazepin-4-yl)acetamido)phenoxy)-9-(3-guanidinopropyl)-1-imino-7,10-dioxo-14,17-dioxa-2,8,11-triazanonadecan-6-yl)carbamate was used, 40.00 mg crude of (S)-2-amino-N—((S)-1-amino-16-(4-(2-((S)-6-(4-chlorophenyl)-8-methoxy-1-methyl-4H-benzo[f][1,2,4]triazolo[4,3-a][1,4]diazepin-4-yl)acetamido)phenoxy)-1-imino-7-oxo-11,14-dioxa-2,8-diazahexadecan-6-yl)-5-guanidinopentanamide was obtained as yellow oil. LC/MS: mass calcd. For C₄₄H₅₉ClN₁₄O₇: 930.44, found: 931.40 [M+H]⁺.

### Step 5: Synthesis of N-(5-((3-((2-(((11S,14S)-1-(4-(2-((S)-6-(4-chlorophenyl)-8-methoxy-1-methyl-4H-benzo[f][1,2,4]triazolo[4,3-a][1,4]diazepin-4-yl)acetamido)phenoxy)-11,14-bis(3-guanidinopropyl)-10,13,16,29-tetraoxo-3,6,19,22,25-pentaoxa-9,12,15,28-tetraazahentriacontan-31-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide. 40.00 mg of (S)-2-amino-N—((S)-1-amino-16-(4-(2-((S)-6-(4-chlorophenyl)-8-methoxy-1-methyl-4H-benzo[f][1,2,4]triazolo[4,3-a][1,4]diazepin-4-yl)acetamido)phenoxy)-1-imino-7-oxo-11,14-dioxa-2,8-diazahexadecan-6-yl)-5-guanidinopentanamide was used, 17.60 mg of N-(5-((3-((2-(((11S,14S)-1-(4-(2-((S)-6-(4-chlorophenyl)-8-methoxy-1-methyl-4H-benzo[f][1,2,4]triazolo[4,3-a][1,4]diazepin-4-yl)acetamido)phenoxy)-11,14-bis(3-guanidinopropyl)-10,13,16,29-tetraoxo-3,6,19,22,25-pentaoxa-9,12,15,28-tetraazahentriacontan-31-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (19.73% yield). HRMS: mass calcd. For C₈₉H₁₁₇ClN₃₀O₁₉: 1944.8800, found: 1945.8893 [M+H]⁺.

Type 10 Synthetic Route to Transcription Modulator Molecules

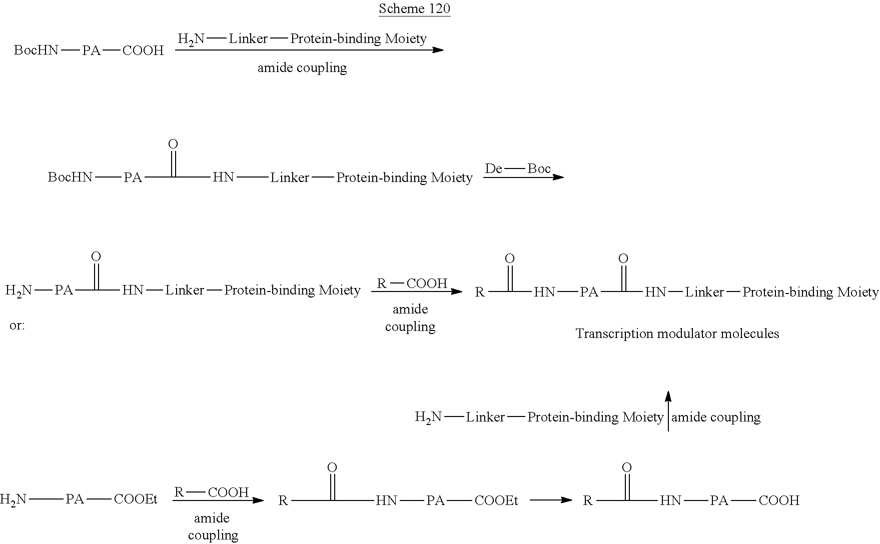

### N-(5-((1-(4-((2S,4R)-1-acetyl-4-((4-chlorophenyl)amino)-2-methyl-1,2,3,4-tetrahydroquinolin-6-yl)phenyl)-1-oxo-5,8,11,14,17,20,23,26-octaoxa-2-azaoctacosan-28-yl)carbamoyl)-1-methyl-1H-pyrrol-3-yl)-4-(3-(4-(4-(3-aminopropanamido)-1-methyl-1H-imidazole-2-carboxamido)-1-methyl-1H-pyrrole-2-carboxamido)propanamido)-1-methyl-1H-imidazole-2-carboxamide (Compound 70)

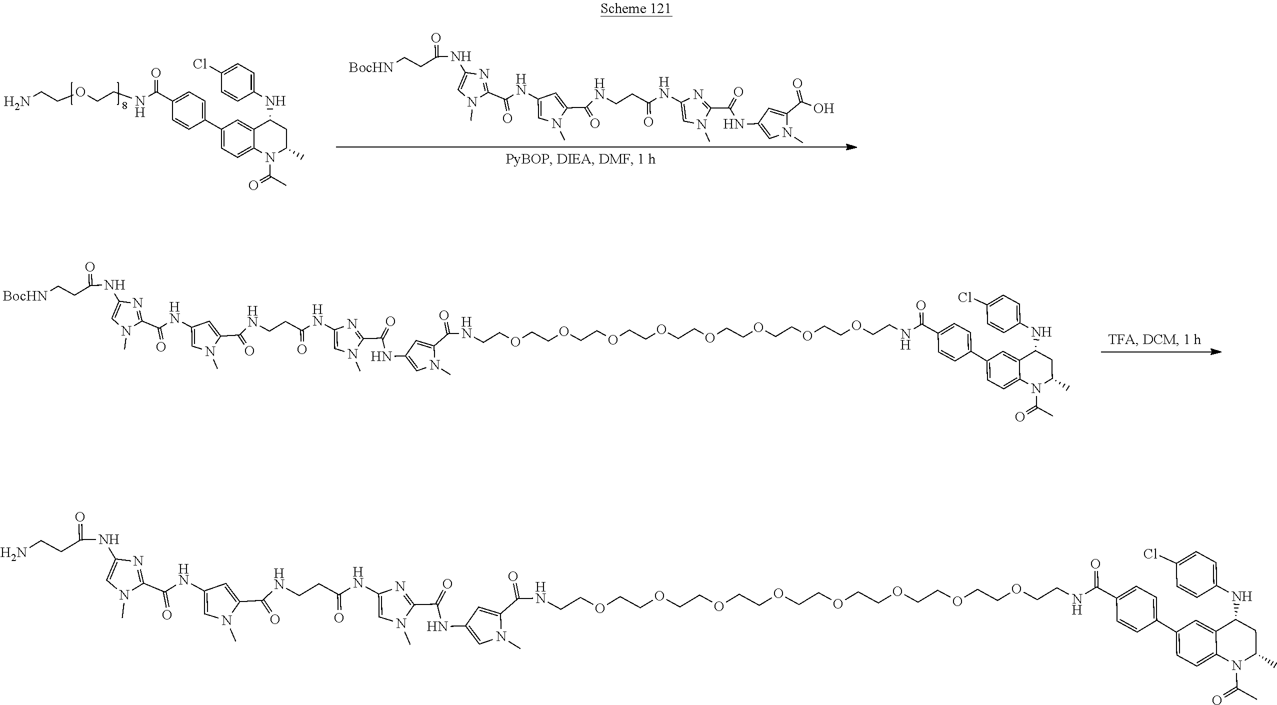

### Step 1: tert-butyl (3-((2-((5-((3-((2-((5-((1-(4-((2S,4R)-1-acetyl-4-((4-chlorophenyl)amino)-2-methyl-1,2,3,4-tetrahydroquinolin-6-yl)phenyl)-1-oxo-5,8,11,14,17,20,23,26-octaoxa-2-azaoctacosan-28-yl)carbamoyl)-1-methyl-1H-pyrrol-3-yl)carbamoyl)-1-methyl-1H-imidazol-4-yl)amino)-3-oxopropyl)carbamoyl)-1-methyl-1H-pyrrol-3-yl)carbamoyl)-1-methyl-1H-imidazol-4-yl)amino)-3-oxopropyl)carbamate

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate. 75.00 mg of 4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrole-2-carboxylic acid was used, 78.00 mg of tert-butyl (3-((2-((5-((3-((2-((5-((1-(4-((2S,4R)-1-acetyl-4-((4-chlorophenyl)amino)-2-methyl-1,2,3,4-tetrahydroquinolin-6-yl)phenyl)-1-oxo-5,8,11,14,17,20,23,26-octaoxa-2-azaoctacosan-28-yl)carbamoyl)-1-methyl-1H-pyrrol-3-yl)carbamoyl)-1-methyl-1H-imidazol-4-yl)amino)-3-oxopropyl)carbamoyl)-1-methyl-1H-pyrrol-3-yl)carbamoyl)-1-methyl-1H-imidazol-4-yl)amino)-3-oxopropyl)carbamate was obtained as yellow solid (43.98% yield). LCMS: mass calcd. For C₇₇H₁₀₇ClN₁₆O₁₇: 1560.72, found: 781.15 [M/2+H]⁺.

### Step 2: Synthesis of N-(5-((1-(4-((2S,4R)-1-acetyl-4-((4-chlorophenyl)amino)-2-methyl-1,2,3,4-tetrahydroquinolin-6-yl)phenyl)-1-oxo-5,8,11,14,17,20,23,26-octaoxa-2-azaoctacosan-28-yl)carbamoyl)-1-methyl-1H-pyrrol-3-yl)-4-(3-(4-(4-(3-aminopropanamido)-1-methyl-1H-imidazole-2-carboxamido)-1-methyl-1H-pyrrole-2-carboxamido)propanamido)-1-methyl-1H-imidazole-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. The product was purified by Prep-HPLC. 50.00 mg of tert-butyl (3-((2-((5-((3-((2-((5-((1-(4-((2S,4R)-1-acetyl-4-((4-chlorophenyl)amino)-2-methyl-1,2,3,4-tetrahydroquinolin-6-yl)phenyl)-1-oxo-5,8,11,14,17,20,23,26-octaoxa-2-azaoctacosan-28-yl)carbamoyl)-1-methyl-1H-pyrrol-3-yl)carbamoyl)-1-methyl-1H-imidazol-4-yl)amino)-3-oxopropyl)carbamoyl)-1-methyl-1H-pyrrol-3-yl)carbamoyl)-1-methyl-1H-imidazol-4-yl)amino)-3-oxopropyl)carbamate was used, 7.30 mg of N-(5-((1-(4-((2S,4R)-1-acetyl-4-((4-chlorophenyl)amino)-2-methyl-1,2,3,4-tetrahydroquinolin-6-yl)phenyl)-1-oxo-5,8,11,14,17,20,23,26-octaoxa-2-azaoctacosan-28-yl)carbamoyl)-1-methyl-1H-pyrrol-3-yl)-4-(3-(4-(4-(3-aminopropanamido)-1-methyl-1H-imidazole-2-carboxamido)-1-methyl-1H-pyrrole-2-carboxamido)propanamido)-1-methyl-1H-imidazole-2-carboxamide was obtained as white solid (16.62% yield). HRMS: mass calcd. For C₇₁H₉₃ClN₁₆O₁₆: 1460.6644, found: 1461.6675 [M+H]⁺.

### N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-(3-carbamimidamidopropanamido)-1-methylimidazole-2-carboxamide (Compound 71)

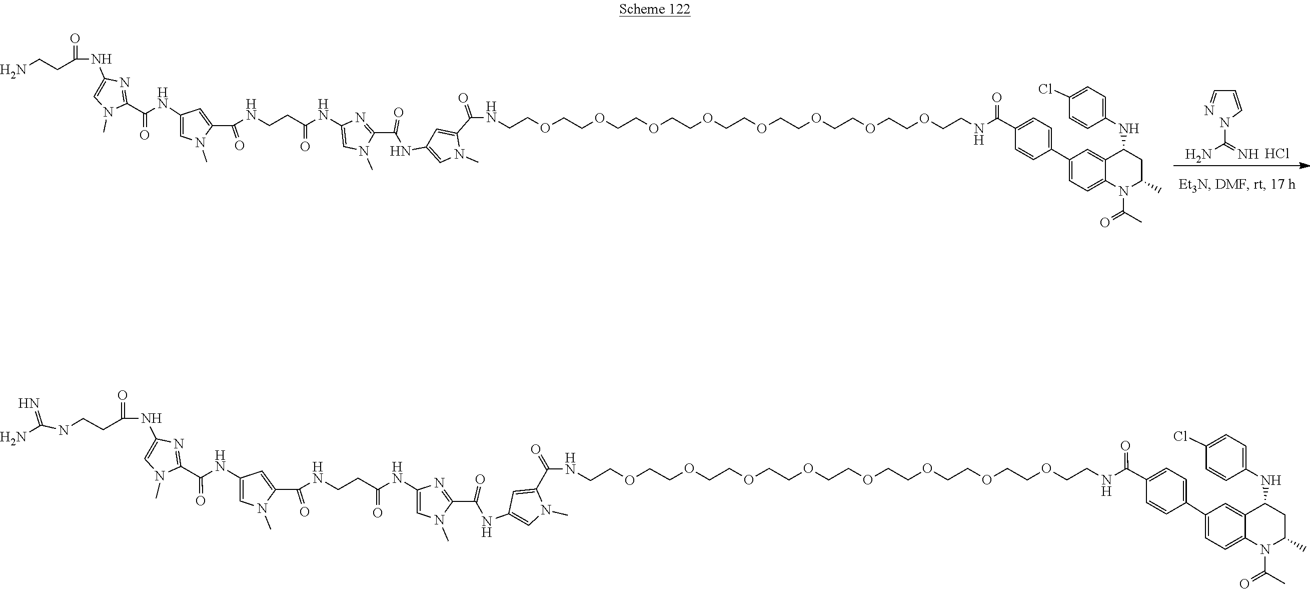

To a stirred solution of N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-(3-aminopropanamido)-1-methylimidazole-2-carboxamide (40.00 mg, 0.03 mmol, 1.00 equiv) and pyrazole-1-carboximidamide (15.00 mg, 0.14 mmol, 5.00 equiv) in DMF (4.00 mL) was added TEA (40.00 mg, 0.40 mmol, 14.45 equiv) in portions. The resulting mixture was stirred for 17 h at room temperature. The reaction mixture was filtered and the filtrate (4.5 mL) was further purified by Prep-HPLC with the following conditions: Column: XBridge Shield RP18 OBD Column, 19*250 mm, 10μm; Mobile Phase A: Water (0.05% TFA), Mobile Phase B: ACN; Flow rate: 25 mL/min; Gradient: 33% B to 39% B in 13 min, 39% B; Wave Length: 254 nm; RT1(min): 11.7. The fractions were combined and lyophilized to afford N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-(3-carbamimidamidopropanamido)-1-methylimidazole-2-carboxamide (10.2 mg, 24.71%) as white solid. HRMS: mass calcd. For C₇₁H₉₃ClN₁₆O₁₆: 1502.6862, found: 1503.6971 [M+H]⁺.

### N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-[3-(2-[2-[2-(2-carbamimidamidoethoxy)ethoxy]ethoxy]acetamido)propanamido]-1-methylimidazole-2-carboxamide (Compound 72)

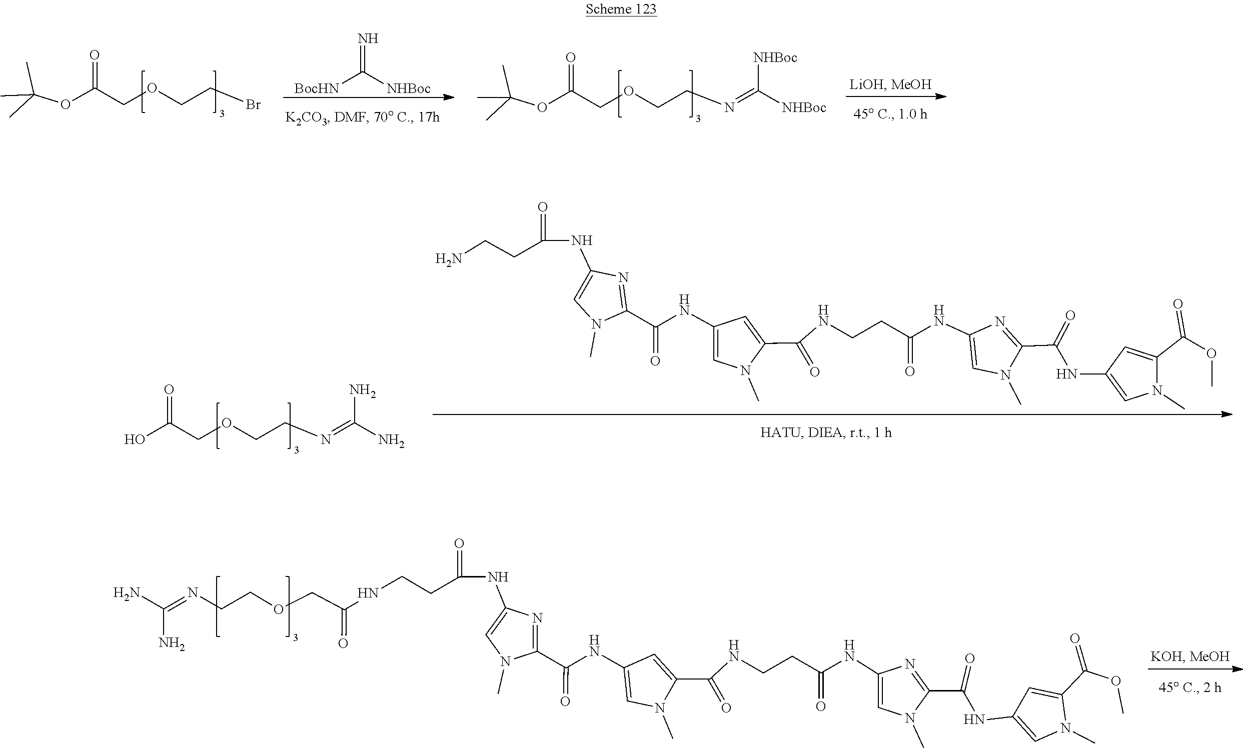

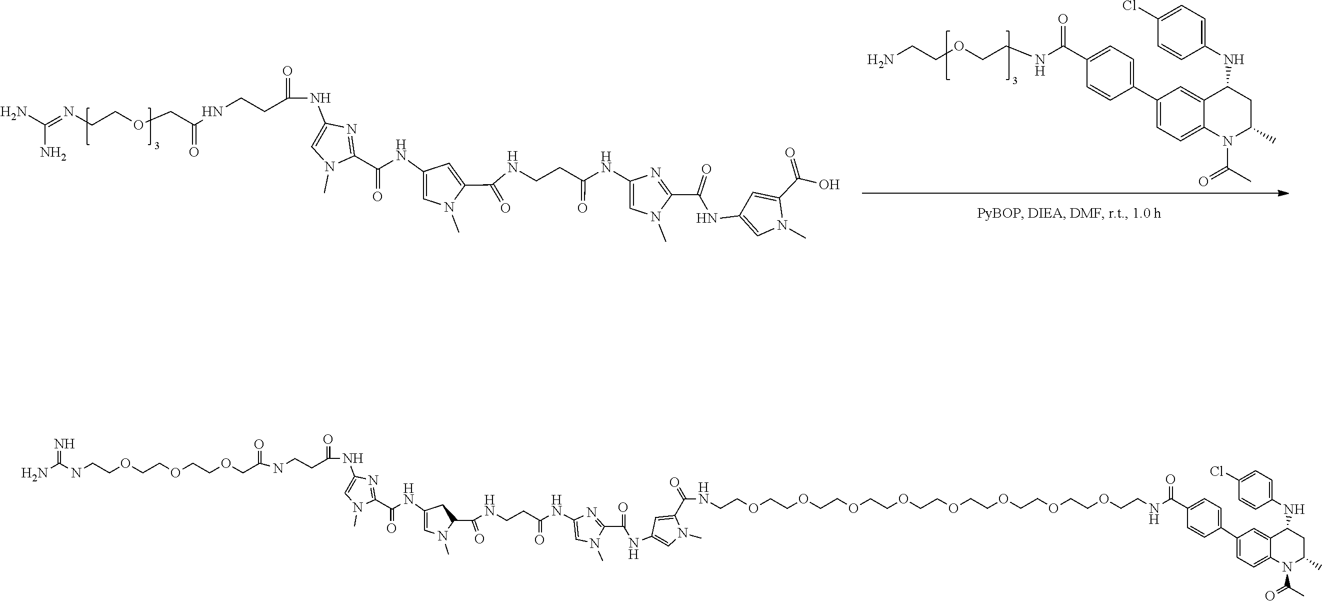

### Step 1: Synthesis of tert-butyl 1,1-bis[(tert-butoxycarbonyl)amino]-5,8,11-trioxa-2-azatridec-1-en-13-oate

To a stirred solution of tert-butyl 2-[2-[2-(2-bromoethoxy)ethoxy]ethoxy]acetate (500.00 mg, 1.53 mmol, 1.00 equiv) in DMF (2.00 mL) was added tert-butyl N-[[(tert-butoxycarbonyl)amino]methanimidoyl]carbamate (396.23 mg, 1.53 mmol, 1.00 equiv) and K₂CO₃ (1900.66 mg, 13.75 mmol, 9.00 equiv) in portions at room temperature. The resulting mixture was stirred for 17 h at 70 degrees C. The solid was filtered out and the filtrate was concentrated. The residue was purified by silica gel column chromatography, eluted with PE:EA=4:1. The fractions were combined and concentrated. Tert-butyl 1,1-bis[(tert-butoxycarbonyl)amino]-5,8,11-trioxa-2-azatridec-1-en-13-oate (420.00 mg, 54.36%) was obtained as a yellow oil. LC/MS: mass calcd. For C₂₃H₄₃N₃O₉: 505.61, found: 506.20 [M+H]⁺.

### Step 2: Synthesis of 1-amino-1-imino-5,8,11-trioxa-2-azatridecan-13-oic acid

The procedure was the same as 4-[3-[(Tert-butoxycarbonyl)amino]propanamido]-1-methylimidazole-2-carboxylic acid, but the product could not be precipitated from H₂O, so the solution was concentrated and the crude solid was used for next step directly. 320.00 mg of tert-butyl 1,1-bis[(tert-butoxycarbonyl)amino]-5,8,11-trioxa-2-azatridec-1-en-13-oate was used, 150.00 mg crude of 1-amino-1-imino-5,8,11-trioxa-2-azatridecan-13-oic acid was obtained as white solid. LC/MS: mass calcd. For C₉H₁₉N₃O₅: 249.13, found: 250.15 [M+H]⁺.

### Step 3: Synthesis of Methyl 4-(4-[3-[(4-[4-[3-(2-[2-[2-(2-carbamimidamidoethoxy) ethoxy]ethoxy]acetamido)propanamido]-1-methylimidazole-2-amido]-1-methylpyrrol-2-yl)formamido]propanamido]-1-methylimidazole-2-amido)-1-methylpyrrole-2-carboxylate

The procedure was the same as ethyl 4-[3-[(tert-butoxycarbonyl)amino]propanamido]-1-methylimidazole-2-carboxylate. 35.00 mg of 1-amino-1-imino-5,8,11-trioxa-2-azatridecan-13-oic acid was used, 80.00 mg of methyl 4-(4-[3-[(4-[4-[3-(2-[2-[2-(2-carbamimidamidoethoxy)ethoxy]ethoxy]acetamido)propanamido]-1-methylimidazole-2-amido]-1-methylpyrrol-2-yl)formamido]propanamido]-1-methylimidazole-2-amido)-1-methylpyrrole-2-carboxylate was obtained as yellow solid (63.56% yield). LC/MS: mass calcd. For C₃₈H₅₃N₁₅O₁₁: 895.40, found: 896.45 [M+H]⁺.

### Step 4: Synthesis of 4-(4-[3-[(4-[4-[3-(2-[2-[2-(2-carbamimidamidoethoxy)ethoxy]ethoxy]acetamido)propanamido]-1-methylimidazole-2-amido]-1-methylpyrrol-2-yl)formamido]propanamido]-1-methylimidazole-2-amido)-1-methylpyrrole-2-carboxylic acid

To a stirred solution of methyl 4-(4-[3-[(4-[4-[3-(2-[2-[2-(2-carbamimidamidoethoxy)ethoxy]ethoxy]acetamido)propanamido]-1-methylimidazole-2-amido]-1-methylpyrrol-2-yl)formamido]propanamido]-1-methylimidazole-2-amido)-1-methylpyrrole-2-carboxylate (50.00 mg, 0.06 mmol, 1.00 equiv) in MeOH (4.00 mL) was added KOH solution (2M, 0.28 mL, 10.00 equiv) dropwise at room temperature. The resulting mixture was stirred for 2 h at 45 degrees C. The solvent was removed and the residue was dissolved in H₂O (1.0 mL), adjusted the pH value to 6 with 2N HCl. The result solution was purified by reverse flash chromatography with the following conditions: column, C18 silica gel; mobile phase, ACN in water (0.05% TFA), 15% to 30% gradient in 10 min; detector, UV 254 nm. The fractions were combined and concentrated. 4-(4-[3-[(4-[4-[3-(2-[2-[2-(2-Carbamimidamidoethoxy)ethoxy]ethoxy]acetamido)propanamido]-1-methylimidazole-2-amido]-1-methylpyrrol-2-yl)formamido]propanamido]-1-methylimidazole-2-amido)-1-methylpyrrole-2-carboxylic acid (35 mg, 71.00% yield) was obtained as yellow oil. LC/MS: mass calcd. For C₃₇H₅₁N₁₅O₁₁: 881.39, found: 882.70 [M+H]⁺.

### Step 5: Synthesis of N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-[3-(2-[2-[2-(2-carbamimidamidoethoxy)ethoxy]ethoxy]acetamido)propanamido]-1-methylimidazole-2-carboxamide

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate, the product was purified by Prep-HPLC. 33.00 mg of 4-(4-[3-[(4-[4-[3-(2-[2-[2-(2-carbamimidamidoethoxy)ethoxy]ethoxy]acetamido)propanamido]-1-methylimidazole-2-amido]-1-methylpyrrol-2-yl)formamido]propanamido]-1-methylimidazole-2-amido)-1-methylpyrrole-2-carboxylic acid was used, 4.50 mg of N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-[3-(2-[2-[2-(2-carbamimidamidoethoxy)ethoxy]ethoxy]acetamido)propanamido]-1-methylimidazole-2-carboxamide was obtained as white solid (6.80% yield). HRMS: mass calcd. For C₈₀H₁₁₀ClN₁₉O₂₀: 1691.7863, found: 1692.8020 [M+H]⁺.

### N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-1-methyl-4-(3-[2-[2-(2-[2-[4-(piperidin-4-ylmethyl)piperazin-1-yl]ethoxy]ethoxy)ethoxy]acetamido]propanamido)imidazole-2-carboxamide (Compound 73)

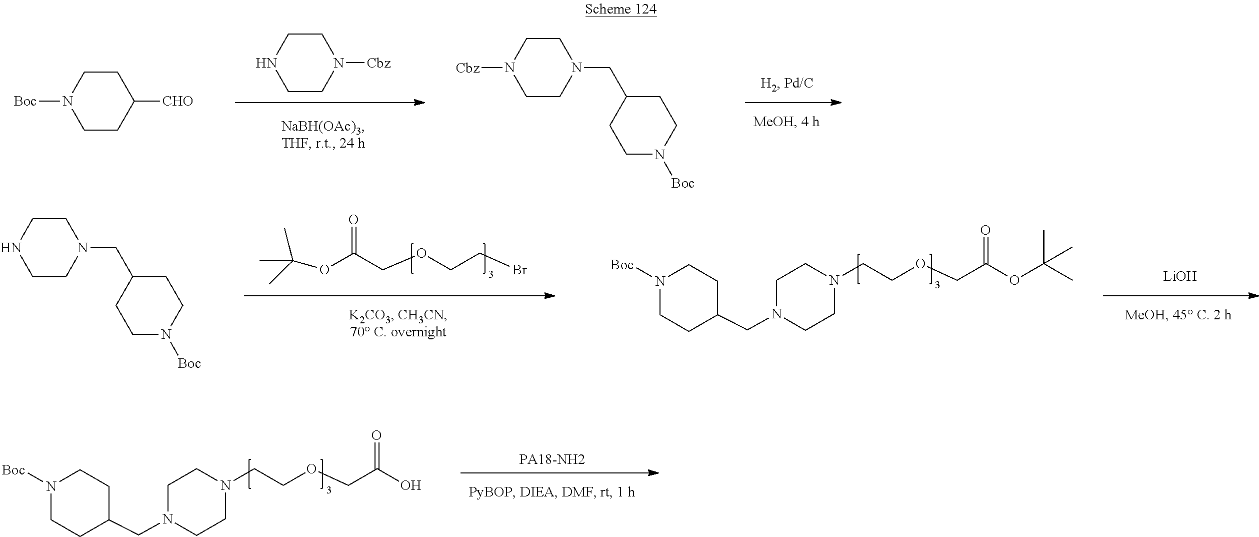

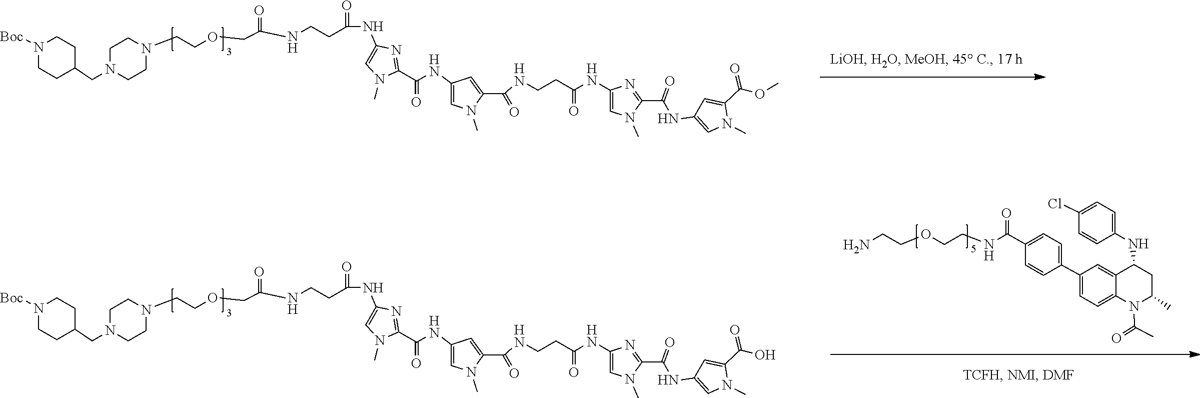

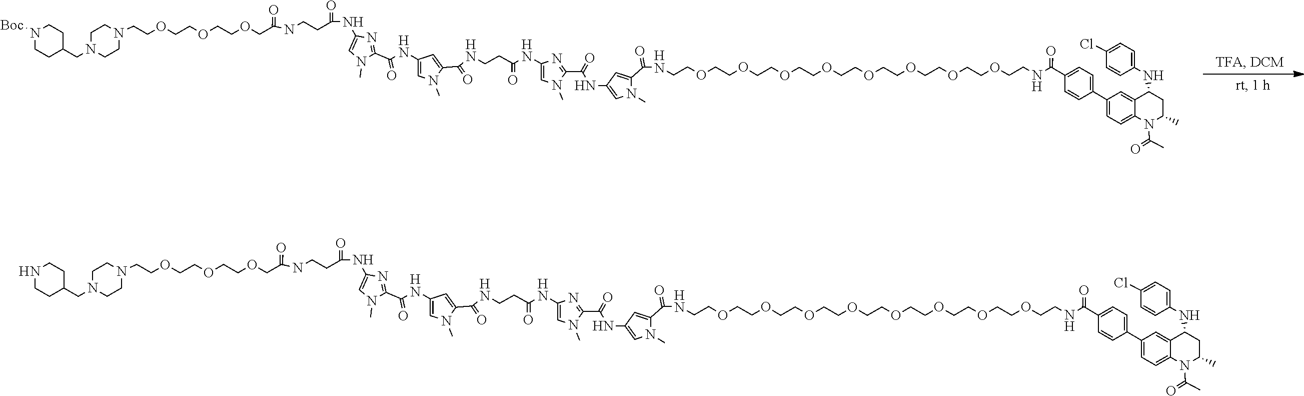

### Step 1: Synthesis of benzyl 4-[[1-(tert-butoxycarbonyl)piperidin-4-yl]methyl]piperazin-1-carboxylate

To a stirred solution of tert-butyl 4-formylpiperidine-1-carboxylate (2.00 g, 9.38 mol, 1.00 equiv) and benzyl piperazine-1-carboxylate (2065.60 mg, 9.38 mmol, 1.00 equiv) in THF (50.00 mL) was added NaBH(OAc)₃ (1987.47 mg, 9.387 mmol, 1.00 equiv). The resulting mixture was stirred for 24 h at room temperature. After reaction, the reaction was quenched with sat.NH₄Cl (20 mL) at 0 degrees C. The resulting mixture was extracted with EA (3×30 mL). The combined organic layers were washed with water (3×10 mL), dried over anhydrous Na₂SO₄. After filtration, the filtrate was concentrated under reduced pressure. The residue was purified by silica gel column chromatography, eluted with PE:EA=1:1 to afford benzyl 4-[[1-(tert-butoxycarbonyl)piperidin-4-yl]methyl]piperazine-1-carboxylate (3.28 g, 83.77%) as light yellow oil. LC/MS: mass calcd. For C₂₃H₃₅N₃O₄: 417.26, found: 418.30 [M+H]⁺.

### Step 2: Synthesis of tert-butyl 4-(piperazin-1-ylmethyl)piperidine-1-carboxylate

To a stirred solution of benzyl 4-[[1-(tert-butoxycarbonyl)piperidin-4-yl]methyl]piperazine-1-carboxylate (2.80 g, 6.71 mmol, 1.00 equiv) in MeOH (30.00 mL) were added Pd/C (560.00 mg, 20% w/w). The resulting mixture was stirred for 4 h at room temperature under H₂ atmosphere. The resulting mixture was filtered, the filter cake was washed with MeOH (3×10 mL). The filtrate was concentrated under reduced pressure. This resulted in tert-butyl 4-(piperazin-1-ylmethyl)piperidine-1-carboxylate (2.00 g, 94.71%) as off-white semi-solid. LCMS: mass calcd. For C₁₅H₂₉N₃O₂: 283.23, found: 284.15 [M+H]⁺.

### Step 3: Synthesis of tert-butyl 4-([4-[2-(2-[2-[2-(tert-butoxy)-2-oxoethoxy]ethoxy]ethoxy)ethyl]piperazin-1-yl]methyl)piperidine-1-carboxylate

The procedure was the same as tert-butyl (S)-(23-(4-(4-(2-(4-(4-chlorophenyl)-2,3,9-trimethyl-6H-thieno[3,2-f][1,2,4]triazolo[4,3-a][1,4]diazepin-6-yl)acetamido)phenyl)piperazin-1-yl)-3,6,9,12,15,18,21-heptaoxatricosyl)carbamate. The product was purified by silica gel column. 500.00 mg of tert-butyl 2-[2-[2-(2-bromoethoxy)ethoxy]ethoxy]acetate was used, 430.00 mg of tert-butyl 4-([42-(2-[2-[2-(tert-butoxy)-2-oxoethoxy]ethoxy]ethoxy)ethyl]piperazin-1-yl]methyl)piperidine-1-carboxylate was obtained as light yellow oil (53.23% yield). LCMS: mass calcd. For C₂₇H₅₁N₃O₇: 529.37, found: 530.35 [M+H]⁺.

### Step 4; Synthesis of (2-[2-[2-(4-[[1-(tert-butoxycarbonyl)piperidin-4-yl]methyl]piperazin-1-yl)ethoxy]ethoxy]ethoxy)acetic acid

The procedure was the same as 4-[3-[(Tert-butoxycarbonyl)amino]propanamido]-1-methylimidazole-2-carboxylic acid, but the product could not be precipitated from H₂O, so the solution was concentrated after acidification and the crude solid was used for next step directly. 410.00 mg of tert-butyl 4-([4-[2-(2-[2-[2-(tert-butoxy)-2-oxoethoxy]ethoxy]ethoxy)ethyl]piperazin-1-yl]methyl)piperidine-1-carboxylate was used, 340.00 mg crude of (2-[2-[2-(4-[[1-(tert-butoxycarbonyl)piperidin-4-yl]methyl]piperazin-1-yl)ethoxy]ethoxy]ethoxy)acetic acid was obtained as off-white solid. LCMS: mass calcd. For C₂₃H₄₃N₃O₇: 473.31, found: 474.20 [M+H]⁺.

### Step 5: Synthesis of tert-butyl 4-([4-[2-(2-[2-[([2-[(2-[[5-([2-[(2-[[5-(methoxycarbonyl)-1-methylpyrrol-3-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)methoxy]ethoxy]ethoxy)ethyl]piperazin-1-yl]methyl)piperidine-1-carboxylate

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate. 70.00 mg of (2-[2-[2-(4-[[1-(tert-butoxycarbonyl)piperidin-4-yl]methyl]piperazin-1-yl)ethoxy]ethoxy]ethoxy)acetic acid was used, 100.00 mg of tert-butyl 4-([4-[2-(2-[2-[([2-[(2-[[5-([2-[(2-[[5-(methoxycarbonyl)-1-methylpyrrol-3-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)methoxy]ethoxy]ethoxy)ethyl]piperazin-1-yl] methyl)piperidine-1-carboxylate was obtained as light yellow solid. LCMS: mass calcd. For C₅₂H₇₇N₁₅O₁₃: 1119.58, found: 1142.80 [M+Na]⁺.

### Step 6: Synthesis of 4-[4-(3-[[4-(4-[3-[2-(2-[2-[2-(4-[[1-(tert-butoxycarbonyl)piperidin-4-yl]methyl]piperazin-1-yl)ethoxy]ethoxy]ethoxy)acetamido]propanamido]-1-methylimidazole-2-amido)-1-methylpyrrol-2-yl]formamido]propanamido)-1-methylimidazole-2-amido]-1-methylpyrrole-2-carboxylic acid

The procedure was the same as 4-[3-[(Tert-butoxycarbonyl)amino]propanamido]-1-methylimidazole-2-carboxylic acid, but the reaction time was 17 h and the product was purified by reverse phase column. 84.00 mg of tert-butyl 4-([4-[2-(2-[2-[([2-[(2-[[5-([2-[(2-[[5-(methoxycarbonyl)-1-methylpyrrol-3-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)methoxy]ethoxy]ethoxy)ethyl]piperazin-1-yl]methyl)piperidine-1-carboxylate was used, 50.00 mg of 4-[4-(3-[[4-(4-[3-[2-(2-[2-[2-(4-[[1-(tert-butoxycarbonyl)piperidin-4-yl]methyl]piperazin-1-yl)ethoxy]ethoxy]ethoxy)acetamido]propanamido]-1-methylimidazole-2-amido)-1-methylpyrrol-2-yl]formamido]propanamido)-1-methylimidazole-2-amido]-1-methylpyrrole-2-carboxylic acid was obtained as light yellow solid (48.22% yield). LCMS: mass calcd. For C₅₁H₇₅N₁₅O₁₃: 1105.57, found: 1106.55 [M+H]⁺.

### Step 7: Synthesis of tert-butyl 4-[[4-(2-[2-[2-([[2-([2-[(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamoyl)ethyl]carbamoyl]methoxy)ethoxy]ethoxy]ethyl)piperazin-1-yl]methyl]piperidine-1-carboxylate

The procedure was the same as (S)—N-(5-((3-((2-((1-(4-(2-(4-(4-chlorophenyl)-2,3,9-trimethyl-6H-thieno[3,2-f][1,2,4] triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide, but the product was obtained via extraction and concentration to obtain the crude of the product. 46.00 mg of 4-[4-(3-[[4-(4-[3-[2-(2-[2-[2-(4-[[1-(tert-butoxycarbonyl)piperidin-4-yl]methyl] piperazin-1-yl)ethoxy]ethoxy]ethoxy)acetamido]propanamido]-1-methylimidazole-2-amido)-1-methylpyrrol-2-yl]formamido]propanamido)-1-methylimidazole-2-amido]-1-methylpyrrole-2-carboxylic acid was used, 40.00 mg crude of tert-butyl 4-[[4-(2-[2-[2-([[2-([2-[(5-[[2-([2-[(5-[[26-([4-[(2S,4R)-1- acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamoyl)ethyl]carbamoyl]methoxy)ethoxy]ethoxy]ethyl)piperazin-1-yl]methyl]piperidine-1-carboxylate was obtained as yellow oil (41.64% yield). LCMS: mass calcd. For C₉₄H₁₃₄ClN₁₉O₂₂: 1915.96, found: 959.60 [M/2+H]⁺.

### Step 8: Synthesis of N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-1-methyl-4-(3-[2-[2-(2-[2-[4-(piperidin-4-ylmethyl)piperazin-1-yl]ethoxy]ethoxy)ethoxy]acetamido]propanamido)imidazole-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide, but the product was purified by Prep-HPLC. 35.00 mg of tert-butyl 4-[[4-(2-[2-[2-([[2-([2-[(5-[[2-([2-[(5-[[26-([4-[(2S,4R)-1-acetyl-4-[(4-chlorophenyl)amino]-2- methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamoyl)ethyl]carbamoyl]methoxy)ethoxy]ethoxy]ethyl)piperazin-1-yl]methyl]piperidine-1-carboxylate was used, 6.20 mg of N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-1-methyl-4-(3-[2-[2-(2-[2-[4-(piperidin-4-ylmethyl)piperazin-1-yl]ethoxy]ethoxy)ethoxy]acetamido]propanamido)imidazole-2-carboxamide was obtained as white solid (18.53% yield). HRMS: mass calcd. For C₈₉H₁₂₆ClN₁₉O₂₀: 1815.9115, found: 1816.9139 [M+H]⁺.

### N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-[3-(47-amino-3,6,9,12,15,18,21,24,27,30,33,36,39,42,45-pentadecaoxaheptatetracontanamido)propanamido]-1-methylimidazole-2-carboxamide (Compound 74)

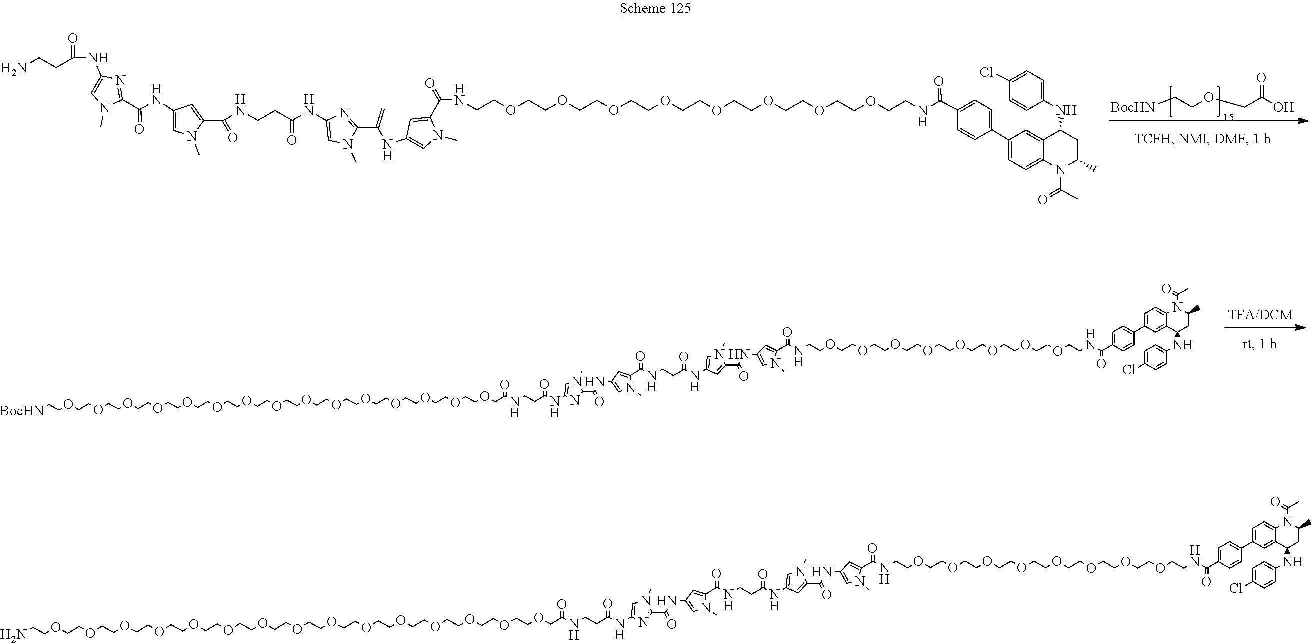

### Step 1: Synthesis of tert-butyl N-(1-[[2-([2-[(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamoyl)ethyl]carbamoyl]-2,5,8,11,14,17,20,23,26,29,32,35,38,41,44-pentadecaoxahexatetracontan-46-yl)carbamate

The procedure was the same as tert-butyl 1-[[4-([[3-(4-methanesulfonyl-2-[6-methyl-7-oxo-1H-pyrrolo[2,3-c]pyridin-4-yl]phenoxy)phenyl]amino]methyl)phenyl]carbamoyl]-2,5,8,11,14-pentaoxaheptadecan-17-oate. 70.00 mg of N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-(3-aminopropanamido)-1-methylimidazole-2-carboxamide was used, 102.00 mg of tert-butyl N-(1-[[2-([2-[(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamoyl)ethyl]carbamoyl]-2,5,8,11,14,17,20,23,26,29,32,35,38,41,44-pentadecaoxahexatetracontan-46-yl)carbamate was obtained as yellow oil (84.10% yield). LCMS: mass calcd. For C₁₀₈H₁₆₄ClN₁₇O₃₄: 2278.13, found: 1141.15 [M/2+H]⁺.

### Step 2: Synthesis of N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-[3-(47-amino-3,6,9,12,15,18,21,24,27,30,33,36,39,42,45-pentadecaoxaheptatetracontanamido)propanamido]-1-methylimidazole-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide, but the product was purified by Prep-HPLC. 60.00 mg of tert-butyl N-(1-[[2-([2-[(5-[[2-([2-[(5-[[26-([4-[(2S,4R)-1-acetyl-4-[(4-chlorophenyl)amino]-2-methyl-3,4-dihydro-2H- quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamoyl)ethyl]carbamoyl]-2,5,8,11,14,17,20,23,26,29,32,35,38,41,44-pentadecaoxahexatetracontan-46-yl)carbamate was used, 9.80 mg of N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-[3-(47-amino-3,6,9,12,15,18,21,24,27,30,33,36,39,42,45-pentadecaoxaheptatetracontanamido)propanamido]-1-methylimidazole-2-carboxamide was obtained as colorless oil (16.43% yield). HRMS: mass calcd. For C₁₀₃H₁₅₆ClN₁₇O₃₂: 2178.0791, found: 2179.0806 [M+H]⁺.

### N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-[3-[(2S)-2-[(2S)-2-amino-5-carbamimidamidopentanamido]-5-carbamimidamidopentanamido]propanamido]-1-methylimidazole-2-carboxamide (Compound 75)

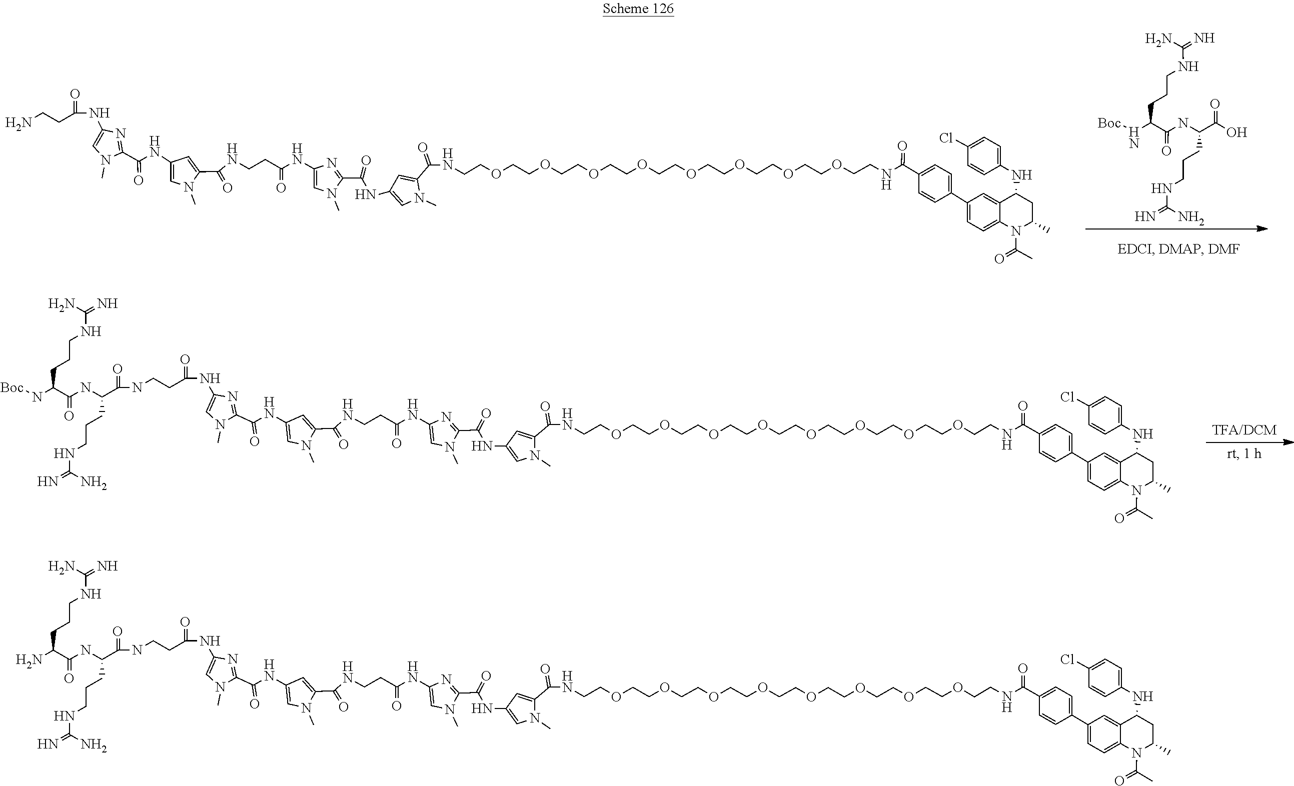

### Step 1: Synthesis of tert-butyl N-[(1S)-1-[[(1S)-1-[[2-([2-[(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamoyl)ethyl]carbamoyl]-4-carbamimidamidobutyl]carbamoyl]-4-carbamimidamidobutyl]carbamate

The procedure was the same as N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide and the product was purified by reverse phase column. 45.00 mg of N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-(3-aminopropanamido)-1-methylimidazole-2-carboxamide was used, 35.00 mg of tert-butyl N-[(1S)-1-[[(1S)-1-[[2-([2-[(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamoyl)ethyl]carbamoyl]-4-carbamimidamidobutyl]carbamoyl]-4-carbamimidamidobutyl]carbamate was obtained as yellow oil (55.81% yield). LCMS: mass calcd. For C₈₈H₁₂₅ClN₂₄O₂₀: 1872.92, found: 938.25 [M/2+H]⁺.

### Step 2: Synthesis of N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-[3-[(2S)-2-[(2S)-2-amino-5-carbamimidamidopentanamido]-5-carbamimidamidopentanamido]propanamido]-1-methylimidazole-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide and the product was purified by Prep-HPLC. 35.00 mg of tert-butyl N-[(1S)-1-[[(1S)-1-[[2-([2-[(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamoyl)ethyl]carbamoyl]-4-carbamimidamidobutyl]carbamoyl]-4-carbamimidamidobutyl]carbamate was used, 11.60 mg of N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-[3-[(2S)-2-[(2S)-2-amino-5-carbamimidamidopentanamido]-5-carbamimidamidopentanamido]propanamido]-1-methylimidazole-2-carboxamide was obtained as white solid (33.43% yield). HRMS: mass calcd. For C₈₃H₁₁₇ClN₂₄O₁₈: 1772.8666, found: 1773.8661 [M+H]⁺.

### N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-(3-aminopropanamido)-1-methylimidazole-2-carboxamide (Compound 77)

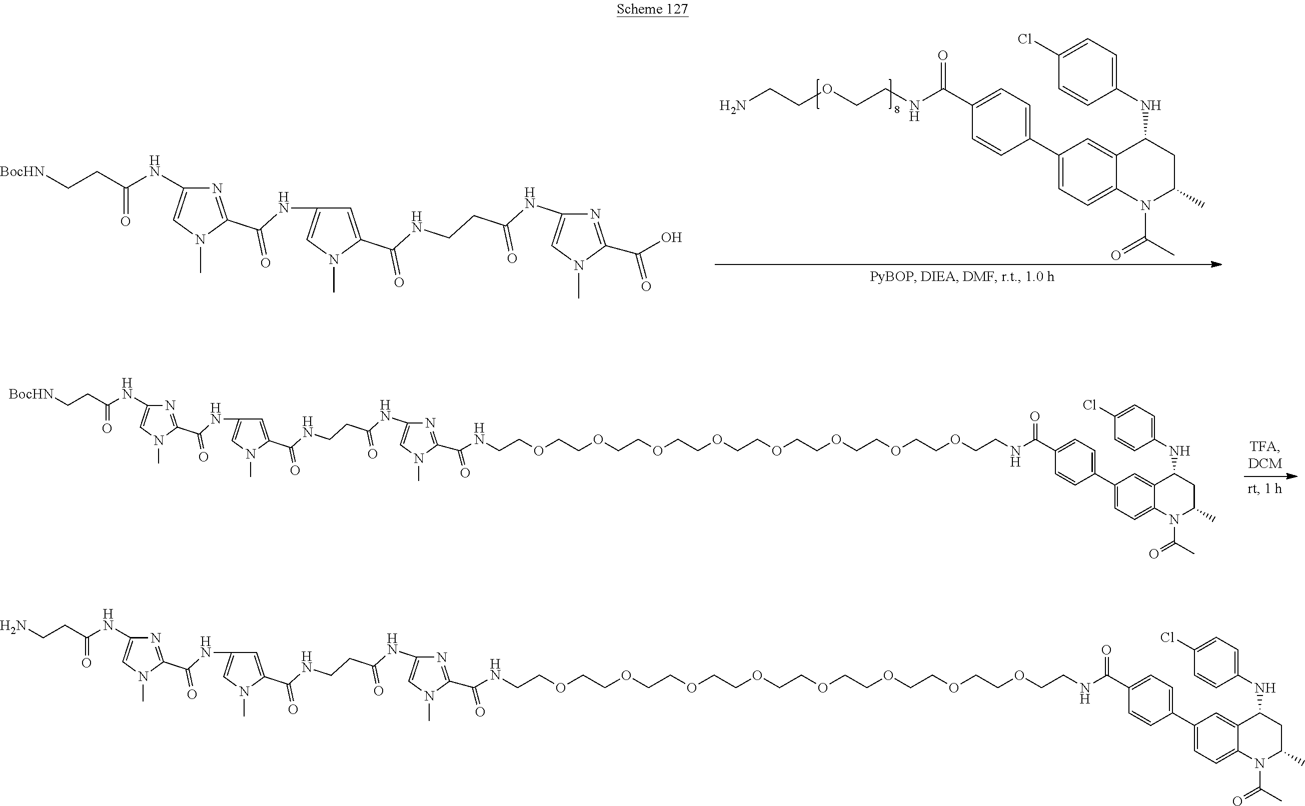

### Step 1: Synthesis of tert-butyl N-[2-[(2-[[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamate

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate. 200.00 mg of 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylic acid was used, 380.00 mg of tert-butyl N-[2-[(2-[[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamate was obtained as yellow solid (71.33% yield). LCMS: mass calcd. For C₇₀H₉₅ClN₁₄O₁₇: 1438.67, found: 721.10 [M/2+H]⁺.

### Step 2: Synthesis of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-(3-aminopropanamido)-1-methylimidazole-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide and the product was purified by Prep-HPLC. 60.00 mg of tert-butyl N-[2-[(2-[[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamate was used, 10.50 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-(3-aminopropanamido)-1-methylimidazole-2-carboxamide was obtained as white solid (18.25% yield). HRMS: mass calcd. For C₆₅H₈₇ClN₁₄O₁₅: 1338.6164, found: 1339.6271 [M+H]⁺.

### N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-(3-carbamimidamidopropanamido)-1-methylimidazole-2-carboxamide (Compound 78)

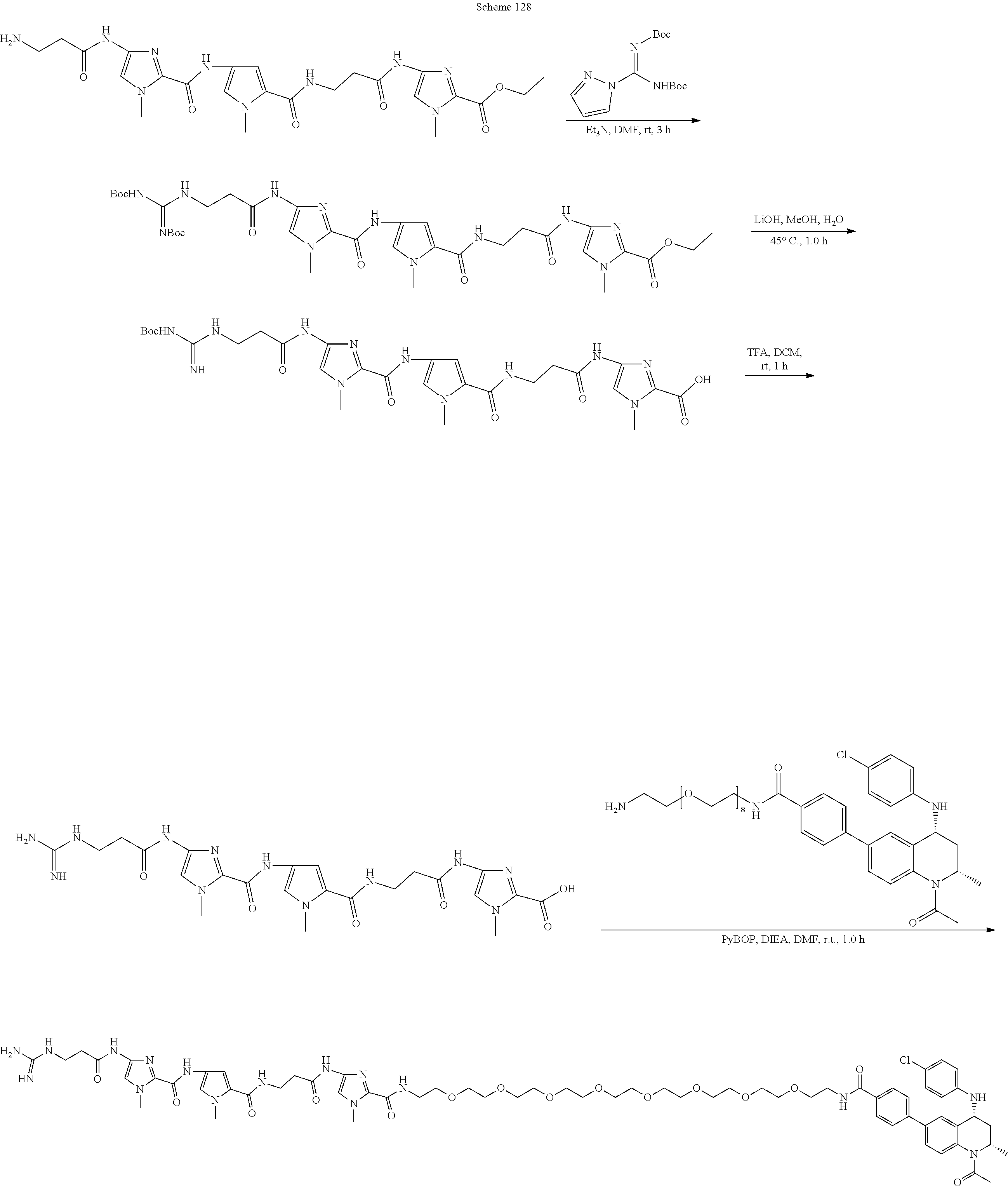

### Step 1: Synthesis of ethyl 4-[3-[(4-[4-[3-([[(tert-butoxycarbonyl)amino][(tert-butoxycarbonyl)imino]methyl]amino)propanamido]-1-methylimidazole-2-amido]-1-methylpyrrol-2-yl)formamido]propanamido]-1-methylimidazole-2-carboxylate

The procedure was the same as N-(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)-4-(3-carbamimidamidopropanamido)-1-methylimidazole-2-carboxamide, but the reaction time was 3 h and the product was purified by silica gel column. 130.00 mg of ethyl 4-[3-([4-[4-(3-aminopropanamido)-1-methylimidazole-2-amido]-1-methylpyrrol-2-yl]formamido)propanamido]-1-methylimidazole-2-carboxylate was used, 122.00 mg of ethyl 4-[3-[(4-[4-[3-([[(tert-butoxycarbonyl)amino][(tert-butoxycarbonyl)imino]methyl]amino)propanamido]-1-methylimidazole-2-amido]-1-methylpyrrol-2-yl)formamido]propanamido]-1-methylimidazole-2-carboxylate was obtained as white solid (67.56% yield). LCMS: mass calcd. For C₃₅H₅₀N₁₂O₁₀: 798.38, found: 799.60 [M+H]⁺.

### Step 2: Synthesis of 4-[3-[(4-[4-[3-([[(tert-butoxycarbonyl)amino]methanimidoyl]amino)propanamido]-1-methylimidazole-2-amido]-1-methylpyrrol-2-yl)formamido]propanamido]-1-methylimidazole-2-carboxylic acid

The procedure was the same as 4-[3-[(Tert-butoxycarbonyl)amino]propanamido]-1-methylimidazole-2-carboxylic acid, and the reaction time was 1 h. 75.00 mg of ethyl 4-[3-[(4-[4-[3-([[(tert-butoxycarbonyl) amino][(tert-butoxycarbonyl)imino]methyl]amino)propanamido]-1-methylimidazole-2-amido]-1-methylpyrrol-2-yl)formamido]propanamido]-1-methylimidazole-2-carboxylate was used, 52.00 mg of 4-[3-[(4-[4-[3-([[(tert-butoxycarbonyl)amino]methanimidoyl]amino)propanamido]-1-methylimidazole-2-amido]-1-methylpyrrol-2-yl)formamido]propanamido]-1-methylimidazole-2-carboxylic acid was obtained as yellow solid (86.20% yield). LCMS: mass calcd. For C₂₈H₃₈N₁₂O₈: 670.29, found: 671.50 [M+H]⁺.

### Step 3: Synthesis of 4-[3-([4-[4-(3-carbamimidamidopropanamido)-1-methylimidazole-2-amido]-1-methylpyrrol-2-yl]formamido)propanamido]-1-methylimidazole-2-carboxylic acid

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 50.00 mg of 4-[3-[(4-[4-[3-([[(tert-butoxycarbonyl)amino]methanimidoyl]amino)propanamido]-1-methylimidazole-2-amido]-1-methylpyrrol-2-yl)formamido]propanamido]-1-methylimidazole-2-carboxylic acid was used, 50.00 mg crude of 4-[3-([4-[4-(3-carbamimidamidopropanamido)-1-methylimidazole-2-amido]-1-methylpyrrol-2-yl]formamido)propanamido]-1-methylimidazole-2-carboxylic acid was obtained as yellow oil. LCMS: mass calcd. For C₂₃H₃₀N₁₂O₆: 570.24, found: 571.40 [M+H]⁺.

### Step 4: Synthesis of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-(3-carbamimidamidopropanamido)-1-methylimidazole-2-carboxamide

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate and the product was purified by Prep-HPLC. 40.00 mg of 4-[3-([4-[4-(3-carbamimidamidopropanamido)-1-methylimidazole-2-amido]-1-methylpyrrol-2-yl]formamido)propanamido]-1-methylimidazole-2-carboxylic acid was used, 11.50 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-(3-carbamimidamidopropanamido)-1-methylimidazole-2-carboxamide was obtained as off-white solid (12.65% yield). HRMS: mass calcd. For C₆₆H₈₉ClN₁₆O₁₅: 1380.6382, found: 1381.6415 [M+H]⁺.

### N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-(2-[2-[2-(2-carbamimidamidoethoxy)ethoxy]ethoxy]acetamido)propanamido]-1-methylimidazole-2-carboxamide (Compound 79)

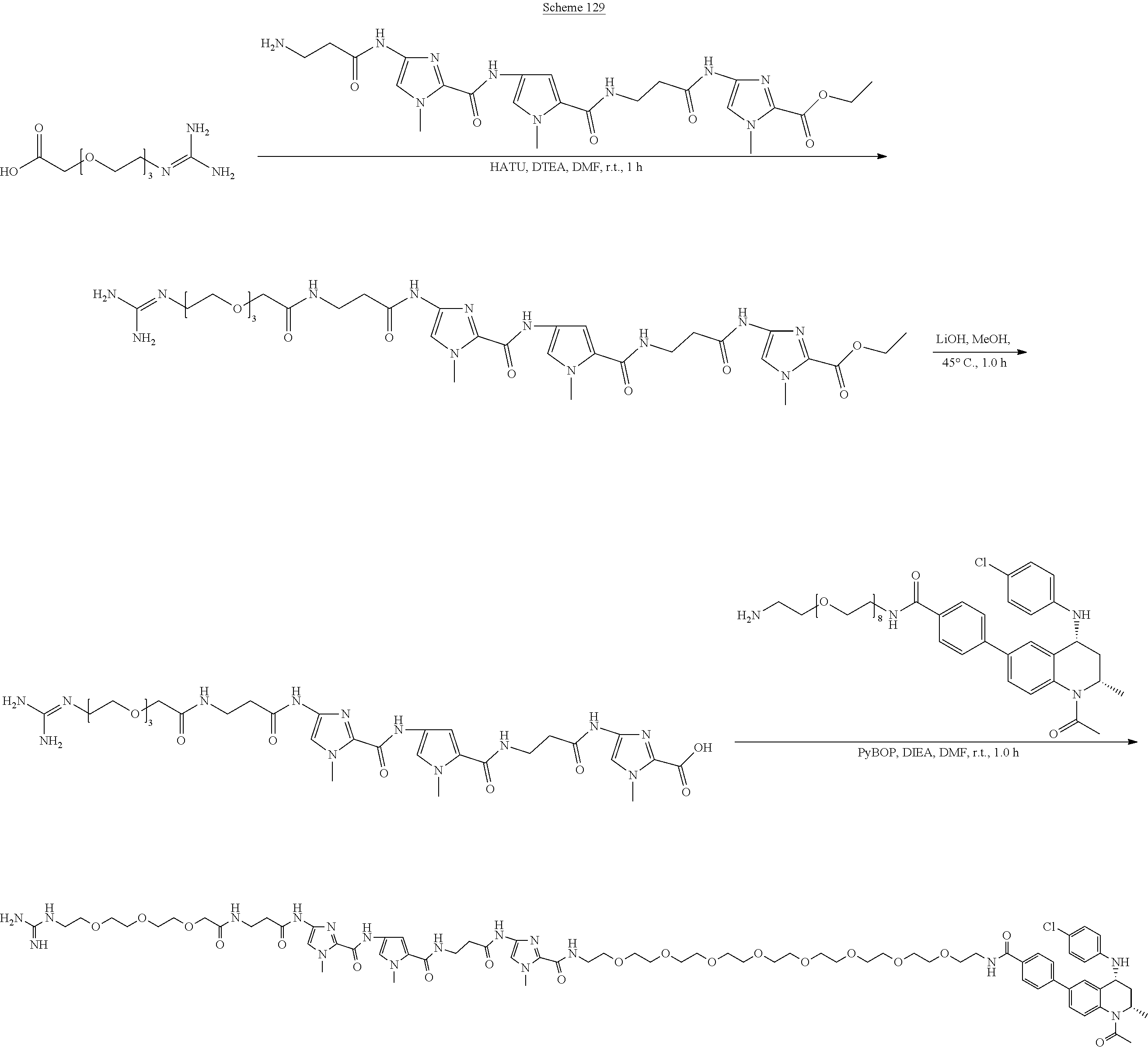

### Step 1: Synthesis of ethyl 4-[3-[(4-[4-[3-(2-[2-[2-(2-carbamimidamidoethoxy)ethoxy]ethoxy]acetamido)propanamido]-1-methylimidazole-2-amido]-1-methylpyrrol-2-yl)formamido]propanamido]-1-methylimidazole-2-carboxylate

The procedure was the same as (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-2,24,34-trioxo-6,9,12,15,18,21-hexaoxa-3,25,29,33-tetraazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. The product was purified by reverse phase column. 40.00 mg of [2-[2-(2-carbamimidamidoethoxy)ethoxy]ethoxy]acetic acid was used, 60.00 mg of ethyl 4-[3-[(4-[4-[3-(2-[2-[2-(2-carbamimidamidoethoxy)ethoxy]ethoxy]acetamido)propanamido]-1-methylimidazole-2-amido]-1-methylpyrrol-2-yl)formamido]propanamido]-1-methylimidazole-2-carboxylate was obtained as light yellow solid (47.62% yield). LC/MS: mass calcd. For C₃₃H₄₉N₁₃O₁₀: 787.37, found: 788.45 [M+H]⁺.

### Step 2: Synthesis of 4-[3-[(4-[4-[3-(2-[2-[2-(2-carbamimidamidoethoxy)ethoxy]ethoxy]acetamido)propanamido]-1-methylimidazole-2-amido]-1-methylpyrrol-2-yl)formamido]propanamido]-1-methylimidazole-2-carboxylic acid

The procedure was the same as 4-[3-[(Tert-butoxycarbonyl)amino]propanamido]-1-methylimidazole-2-carboxylic acid and the product was purified by reverse phase column. 45.00 mg of ethyl 4-[3-[(4-[4-[3-(2-[2-[2-(2-carbamimidamidoethoxy)ethoxy]ethoxy]acetamido)propanamido]-1-methylimidazole-2-amido]-1-methylpyrrol-2-yl)formamido]propanamido]-1-methylimidazole-2-carboxylate was used, 39.00 mg of 4-[3-[(4-[4-[3-(2-[2-[2-(2-carbamimidamidoethoxy)ethoxy]ethoxy]acetamido)propanamido]-1-methylimidazole-2-amido]-1-methylpyrrol-2-yl)formamido]propanamido]-1-methylimidazole-2-carboxylic acid was obtained as yellow oil (89.47% yield). LC/MS: mass calcd. For C₃₁H₄₅N₁₃O₁₀: 759.34, found: 380.80 [M/2+H]⁺.

### Step 3: Synthesis of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-(2-[2-[2-(2-carbamimidamidoethoxy)ethoxy]ethoxy]acetamido)propanamido]-1-methylimidazole-2-carboxamide

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate and the product was purified by Prep-HPLC. 34.00 mg of 4-[3-[(4-[4-[3-(2-[2-[2-(2-carbamimidamidoethoxy)ethoxy]ethoxy]acetamido)propanamido]-1-methylimidazole-2-amido]-1-methylpyrrol-2-yl)formamido]propanamido]-1-methylimidazole-2-carboxylic acid was used, 2.30 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-(2-[2-[2-(2-carbamimidamidoethoxy)ethoxy]ethoxy]acetamido)propanamido]-1-methylimidazole-2-carboxamide was obtained as white solid (3.19% yield). HRMS: mass calcd. For C₇₄H₁₀₄ClN₁₇O₁₉: 1569.7383, found: 1570.7457 [M+H]⁺.

### N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-1-methyl-4-(3-[2-[2-(2-[2-[4-(piperidin-4-ylmethyl)piperazin-1-yl]ethoxy]ethoxy)ethoxy]acetamido]propanamido)imidazole-2-carboxamide (Compound 80)

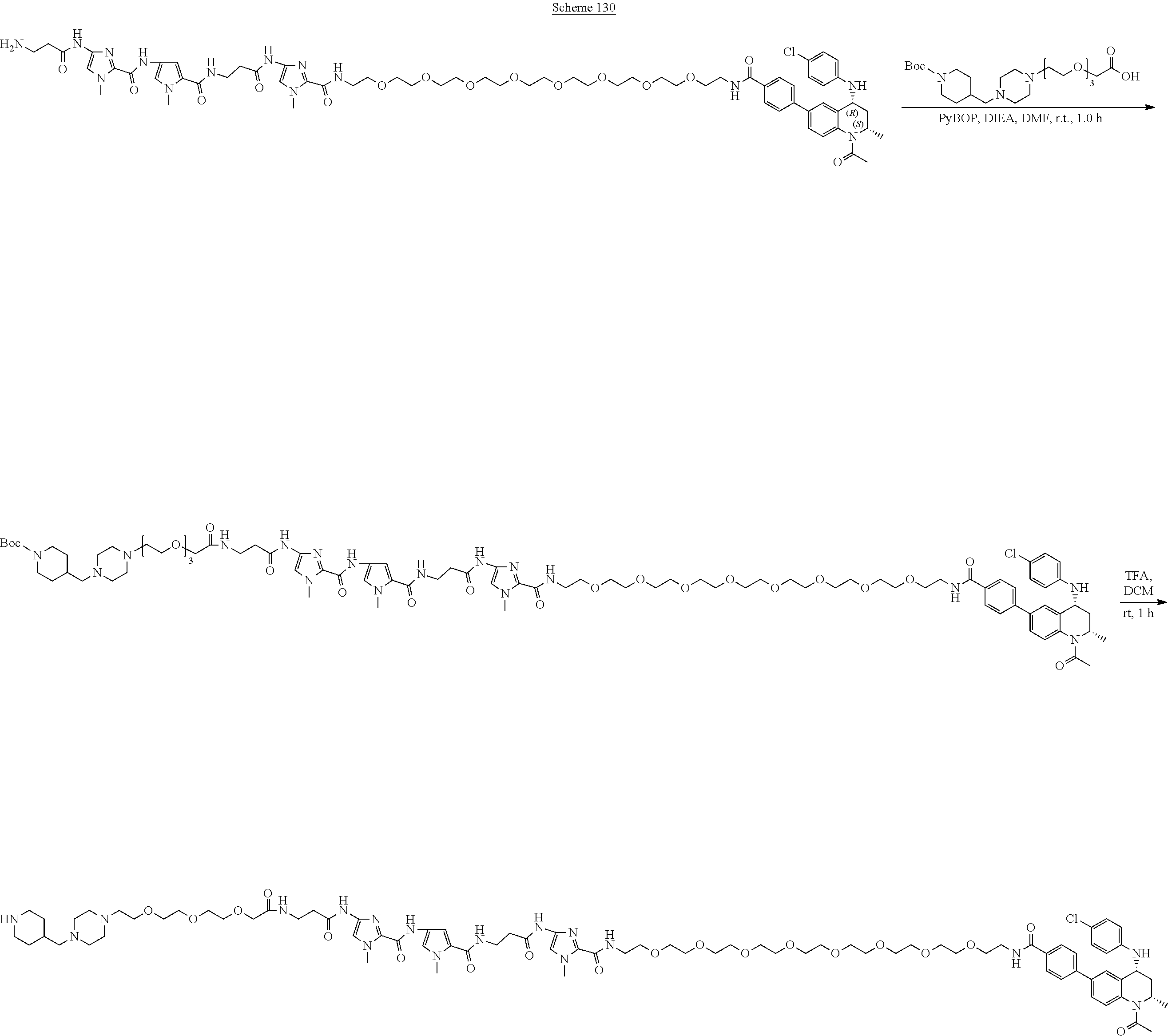

### Step 1: Synthesis of tert-butyl 4-([4-[2-(2-[[2-[2-([2-1(2-[5-([2-1(2-[[26-([4-(2S,4R)-1- acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)methoxy]ethoxy]ethoxy)ethyl]piperazin-1-yl]methyl)piperidine-1-carboxylate

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate. 50.00 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-(3-aminopropanamido)-1-methylimidazole-2-carboxamide was used, 50.00 mg of tert-butyl 4-([4-[2-(2-[2-[([2-[(2-[[5-([2-[(2-[[26-([4-[(2S,4R)-1-acetyl-4-[(4-chlorophenyl)amino]-2-methyl-3,4- dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)methoxy]ethoxy]ethoxy)ethyl]piperazin-1-yl]methyl)piperidine-1-carboxylate was obtained as yellow oil (64.18% yield). LCMS: mass calcd. For C₈₈H₁₂₈ClN₁₇O₂₁: 1793.92, found: 898.90 [M/2+H]⁺.

### Step 2: Synthesis of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-1-methyl-4-(3-[2-[2-(2-[2-[4-(piperidin-4-ylmethyl)piperazin-1-yl]ethoxy]ethoxy)ethoxy]acetamido]propanamido)imidazole-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide, but the product was purified by Prep-HPLC. 55.00 mg of tert-butyl 4-([4-[2-(2-[2-[([2-[(2-[[5-([2-[(2-[[26-([4-[(2S,4R)-1-acetyl-4-[(4-chlorophenyl)amino]-2-methyl-3,4- dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)methoxy]ethoxy]ethoxy)ethyl]piperazin-1-yl]methyl)piperidine-1-carboxylate was used, 10.20 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-1-methyl-4-(3-[2-[2-(2-[2-[4-(piperidin-4-ylmethyl)piperazin-1-yl]ethoxy]ethoxy)ethoxy]acetamido]propanamido)imidazole-2-carboxamide as was obtained as white solid (21.70% yield). HRMS: mass calcd. For C₈₃H₁₂₀ClN₁₇O₁₉: 1693.8635, found: 1694.8671 [M+H]⁺.

### N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-(47-amino-3,6,9,12,15,18,21,24,27,30,33,36,39,42,45-pentadecaoxaheptatetracontanamido)propanamido]-1-methylimidazole-2-carboxamide (Compound 81)

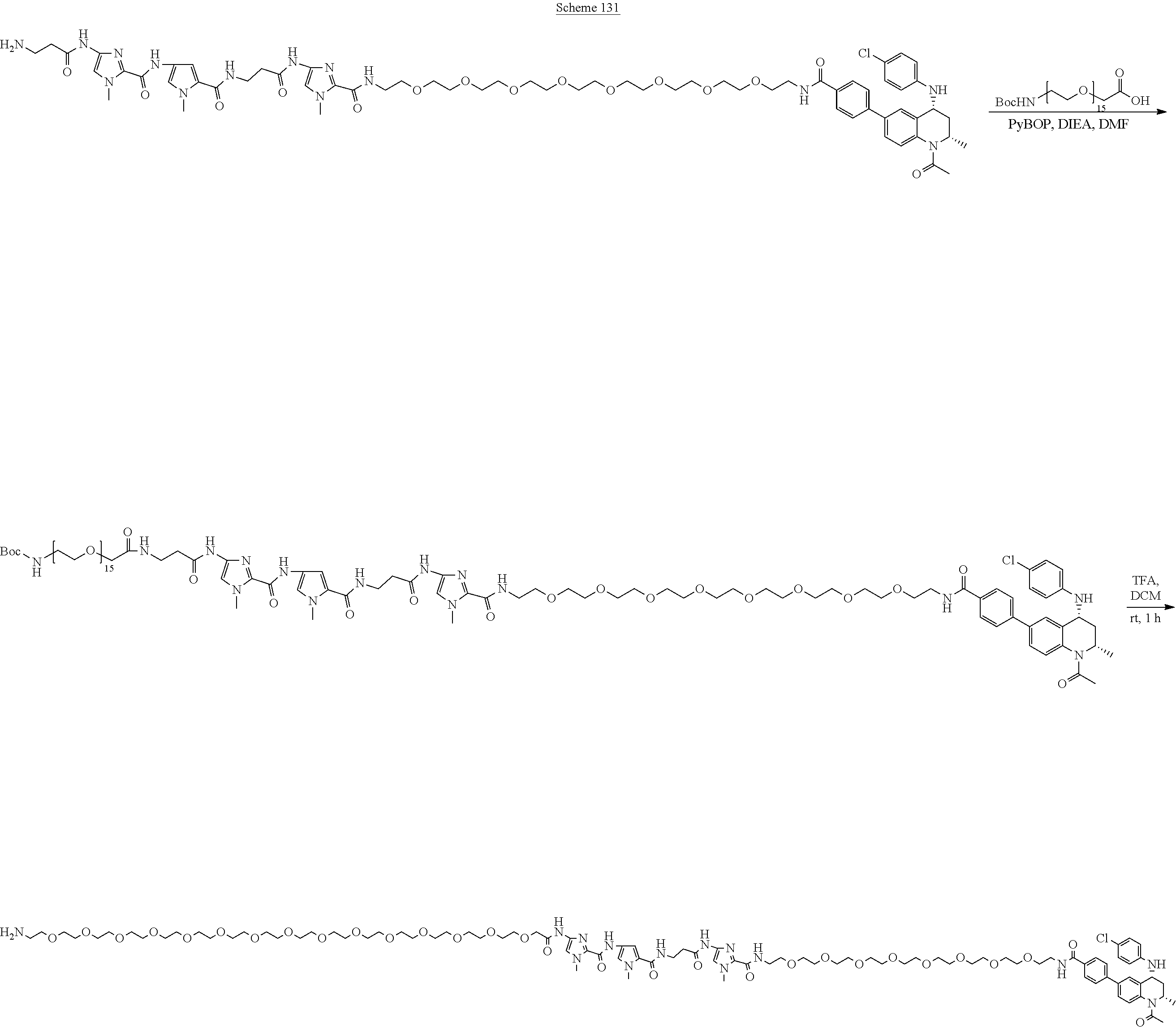

### Step 1: Synthesis of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-(47-amino-3,6,9,12,15,18,21,24,27,30,33,36,39,42,45-pentadecaoxaheptatetracontanamido)propanamido]-1-methylimidazole-2-carboxamide

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate. 45.00 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-(3-aminopropanamido)-1-methylimidazole-2-carboxamide was used, 40.00 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-(47-amino-3,6,9,12,15,18,21,24,27,30,33,36,39,42,45-pentadecaoxaheptatetracontanamido)propanamido]-1-methylimidazole-2-carboxamide was obtained as yellow oil (54.99% yield). LCMS: mass calcd. For C₁₀₂H₁₅₈ClN₁₅O₃₃: 2156.08, found: 1079.65 [M/2+H]⁺

### Step 2: Synthesis of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-(47-amino-3,6,9,12,15,18,21,24,27,30,33,36,39,42,45-pentadecaoxaheptatetracontanamido)propanamido]-1-methylimidazole-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide, but the product was purified by Prep-HPLC. 35.00 mg of tert-butyl N-[1-([2-[(2-[[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-2,5,8,11,14,17,20,23,26,29,32,35,38,41,44-pentadecaoxahexatetracontan-46-yl]carbamate was used, 12.80 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-(47-amino-3,6,9,12,15,18,21,24,27,30,33,36,39,42,45-pentadecaoxaheptatetracontanamido)propanamido]-1-methylimidazole-2-carboxamide was obtained as colorless oil. HRMS: mass calcd. For C₉₇H₁₅₀ClN₁₅O₃₁: 2056.0311, found: 2057.0349 [M+H]⁺.

### N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-[(2S)-2-[(2S)-2-amino-5-carbamimidamidopentanamido]-5-carbamimidamidopentanamido]propanamido]-1-methylimidazole-2-carboxamide (Compound 82)

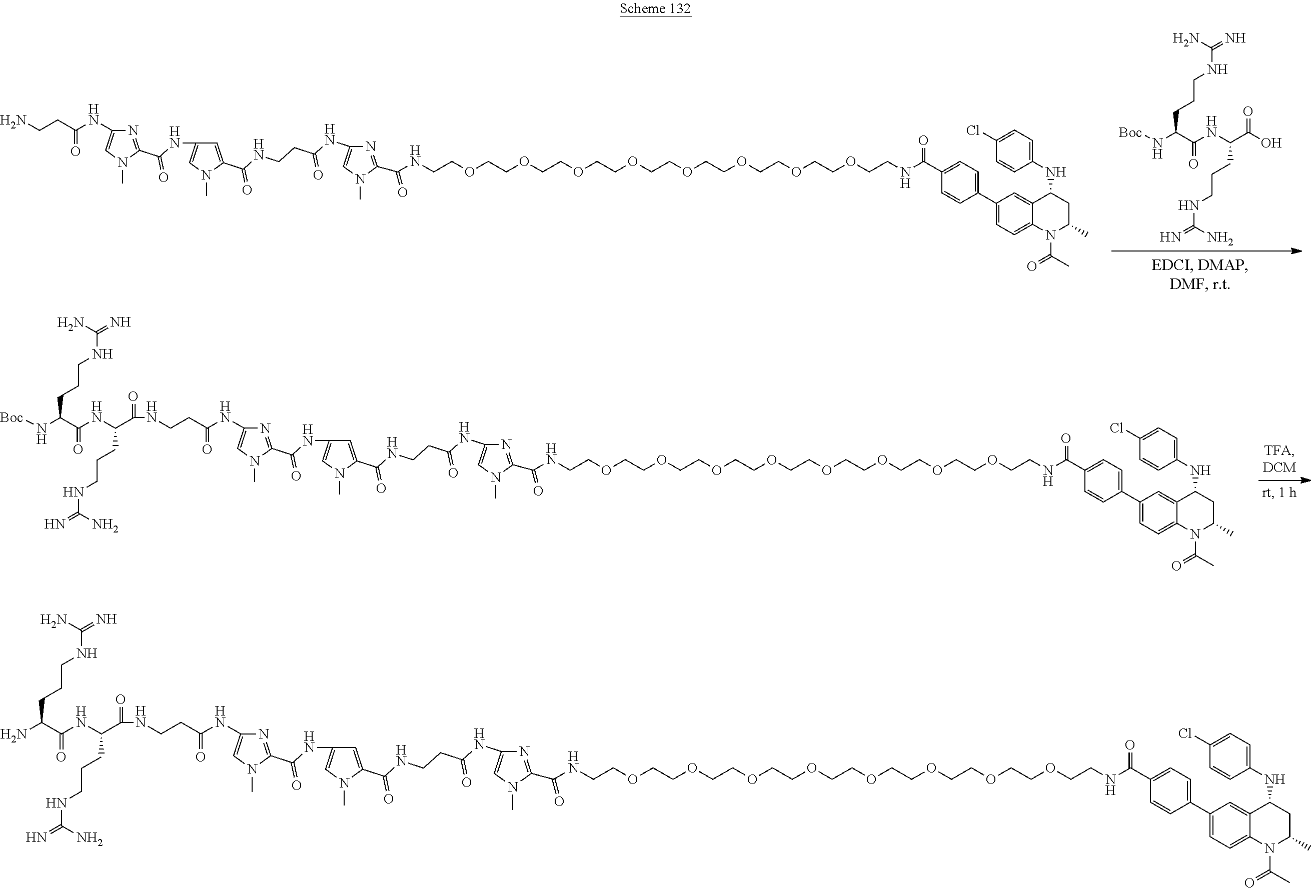

### Step 1: Synthesis of tert-butyl N-[(1S)-1-[[(1S)-1-([2-[(2-[[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-4-carbamimidamidobutyl]carbamoyl]-4-carbamimidamidobutyl]carbamate

The procedure was the same as N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide and the product was purified by reverse phase column. 60.00 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-(3-aminopropanamido)-1-methylimidazole-2-carboxamide was used, 50.00 mg of tert-butyl N-[(1S)-1-[[(1S)-1-([2-[(2-[[5-([2-[(2-[[26-([4-[(2S,4R)-1-acetyl-4-[(4-chlorophenyl)amino]-2-methyl- 3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-4-carbamimidamidobutyl]carbamoyl]-4-carbamimidamidobutyl]carbamate was obtained as yellow oil (58.62% yield). LCMS: mass calcd. For C₈₂H₁₁₉ClN₂₂O₁₉: 1750.87, found: 877.20 [M/2+H]⁺.

### Step 2: Synthesis of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-[(2S)-2-[(2S)-2-amino-5-carbamimidamidopentanamido]-5-carbamimidamidopentanamido]propanamido]-1-methylimidazole-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide, but the product was purified by Prep-HPLC. 50.00 mg of tert-butyl N-[(1S)-1-[[(1S)-1-([2-[(2-[[5-([2-[(2-[[26-([4-[(2S,4R)-1-acetyl-4-[(4-chlorophenyl)amino]-2-methyl- 3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-4-carbamimidamidobutyl]carbamoyl]-4-carbamimidamidobutyl]carbamate was used, 15.8 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-[(2S)-2-[(2S)-2-amino-5-carbamimidamidopentanamido]-5-carbamimidamidopentanamido]propanamido]-1-methylimidazole-2-carboxamide was obtained as white solid (36.32% yield). HRMS: mass calcd. For C₇₇H₁₁₁ClN₂₂O₁₇: 1650.8186, found: 1651.8245 [M+H]⁺.

### N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-([4-[4-(3-aminopropanamido)-1-methylimidazole-2-amido]-1-methylpyrrol-2-yl]formamido)propanamido]-1-methylimidazole-2-carboxamide (Compound 85)

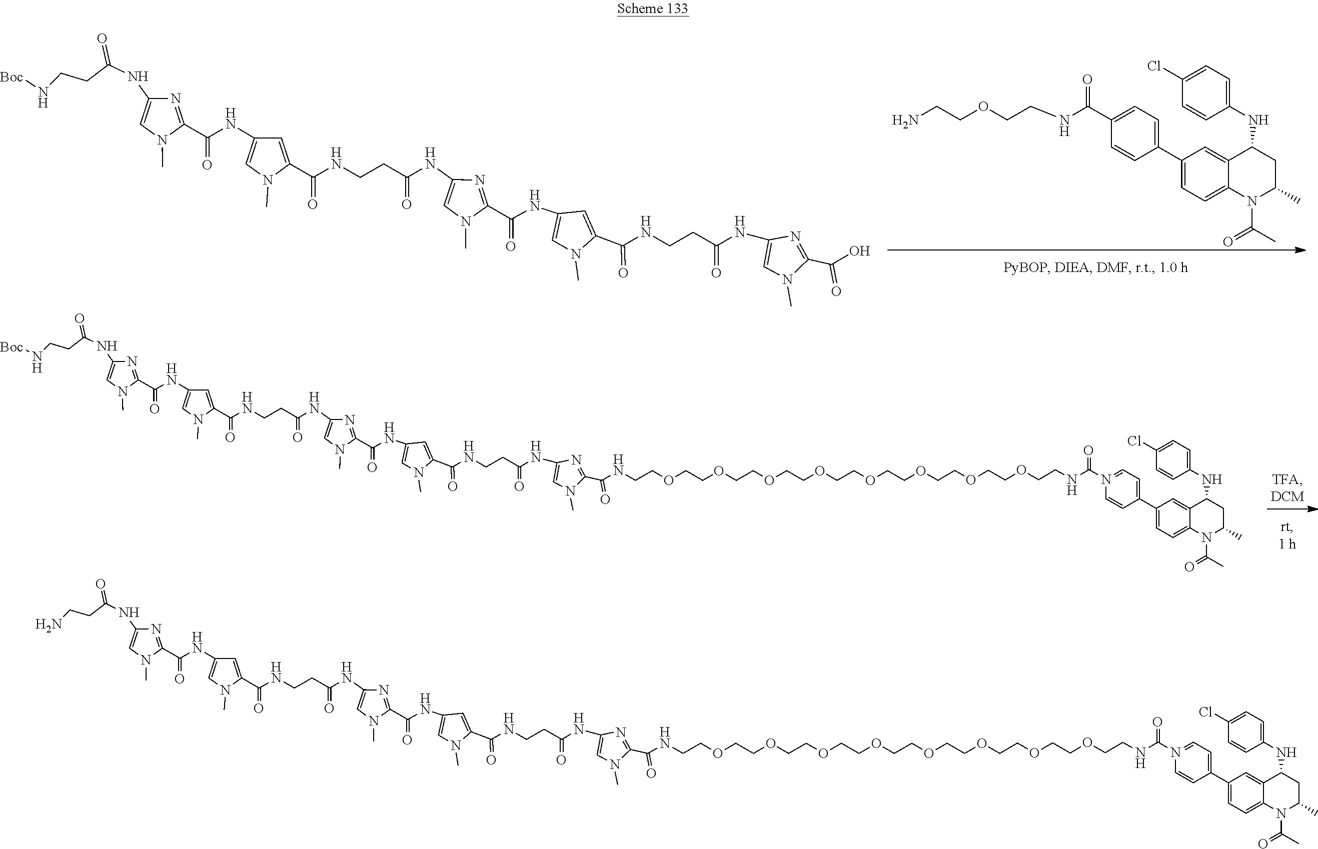

### Step 1: Synthesis of tert-butyl N-[2-[(2-[5-([2-[(2-[5-([2-[(2-[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamate

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate. 60.00 mg of 4-[3-([4-[4-(3-[[4-(4-[13-[(tert-butoxycarbonyl)amino]propanamido]-1-methylimidazole-2-amido)-1-methylpyrrol-2-yl]formamido]propanamido)-1-methylimidazole-2-amido]-1-methylpyrrol-2-yl]formamido)propanamido]-1-methylimidazole-2-carboxylic acid was used, 65.00 mg of tert-butyl N-[12-[(2-[[5-([2-[(2-[[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamate was obtained as yellow oil (52.46% yield). LCMS: mass calcd. For C₈₄H₁₁₁ClN₂₀O₂₀: 1754.80, found: 879.10 [M/2+H]⁺.

### Step 2; Synthesis of N-[5-([2-[(2-[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-([4-[4-(3-aminopropanamido)-1-methylimidazole-2-amido]-1-methylpyrrol-2-yl]formamido)propanamido]-1-methylimidazole-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide, but the product was purified by Prep-HPLC. 60.00 mg of tert-butyl N-[2-[(2-[[5-([2-[(2-[[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamate was used, 8.70 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-([4-[4-(3-aminopropanamido)-1-methylimidazole-2-amido]-1-methylpyrrol-2-yl]formamido)propanamido]-1-methylimidazole-2-carboxamide was obtained as white solid (14.67% yield). HRMS: mass calcd. For C₇₉H₁₀₃ClN₂₀O₁₈: 1654.7448, found: 1655.7561 [M+H]⁺.

### N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-([4-[4-(3-carbamimidamidopropanamido)-1-methylimidazole-2-amido]-1-methylpyrrol-2-yl]formamido)propanamido]-1-methylimidazole-2-carboxamide (Compound 86)

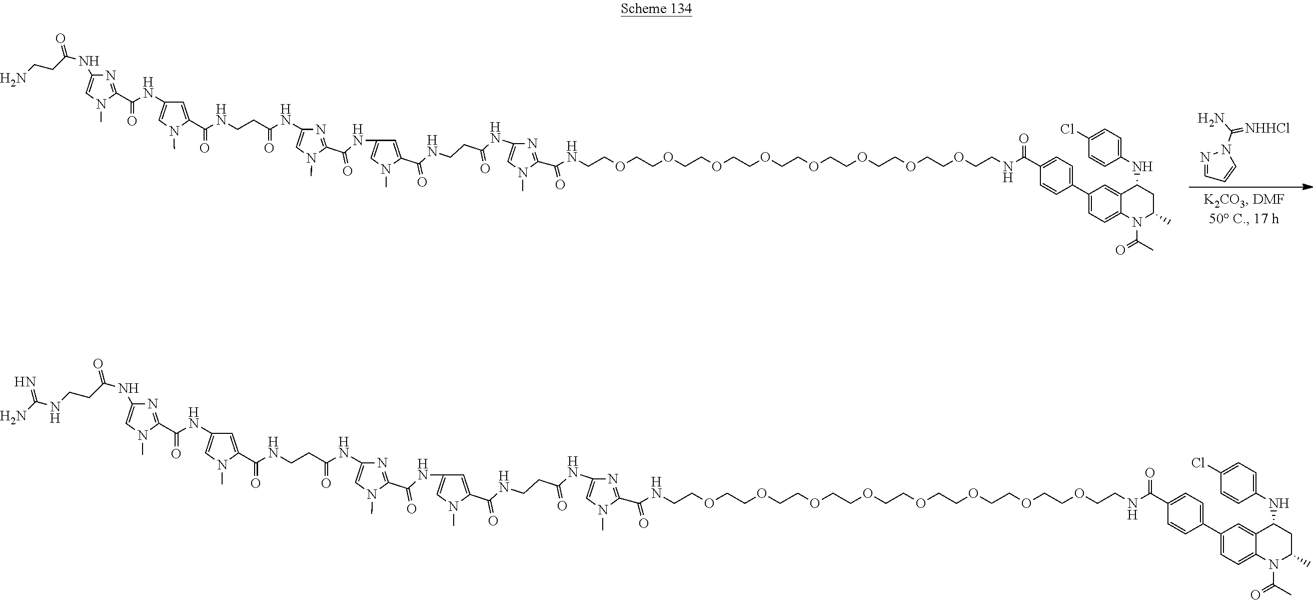

To a stirred solution of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-([4-[4-(3-aminopropanamido)-1-methylimidazole-2-amido]-1-methylpyrrol-2-yl]formamido)propanamido]-1-methylimidazole-2-carboxamide (40.00 mg, 0.02 mmol, 1.00 equiv) in DMF (1.00 mL) was added K₂CO₃ (32.70 mg, 0.236 mmol, 10.00 equiv) and pyrazole-1-carboximidamide hydrochloride (14.00 mg, 0.10 mmol, 5.00 equiv) in portions at room temperature. The resulting mixture was stirred for 17 h at 50 degree C. The solid was filtered out and the filtrate was purified by Prep-HPLC with the following conditions: Column: XBridge Shield RP18 OBD Column, 19*250 mm, 10 um; Mobile Phase A: Water (0.05% TFA), Mobile Phase B: ACN; Flow rate: 25 mL/min; Gradient: 33 B to 39B in 13 min; 254 nm; RT1: 12.18. The fractions were combined and lyophilized. N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-([4-[4-(3-carbamimidamidopropanamido)-1-methylimidazole-2-amido]-1-methylpyrrol-2-yl]formamido)propanamido]-1-methylimidazole-2-carboxamide (4.5 mg, 10.66%) was obtained as white solid. HRMS: mass calcd. For C₈₀H₁₀₅ClN₂₂O₁₈: 1696.7666 found: 1697.7723 [M+H]⁺.

### N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-[(4-[4-[3-(2-[2-[2-(2-carbamimidamidoethoxy)ethoxy]ethoxy]acetamido)propanamido]-1-methylimidazole-2-amido]-1-methylpyrrol-2-yl)formamido]propanamido]-1-methylimidazole-2-carboxamide (Compound 87)

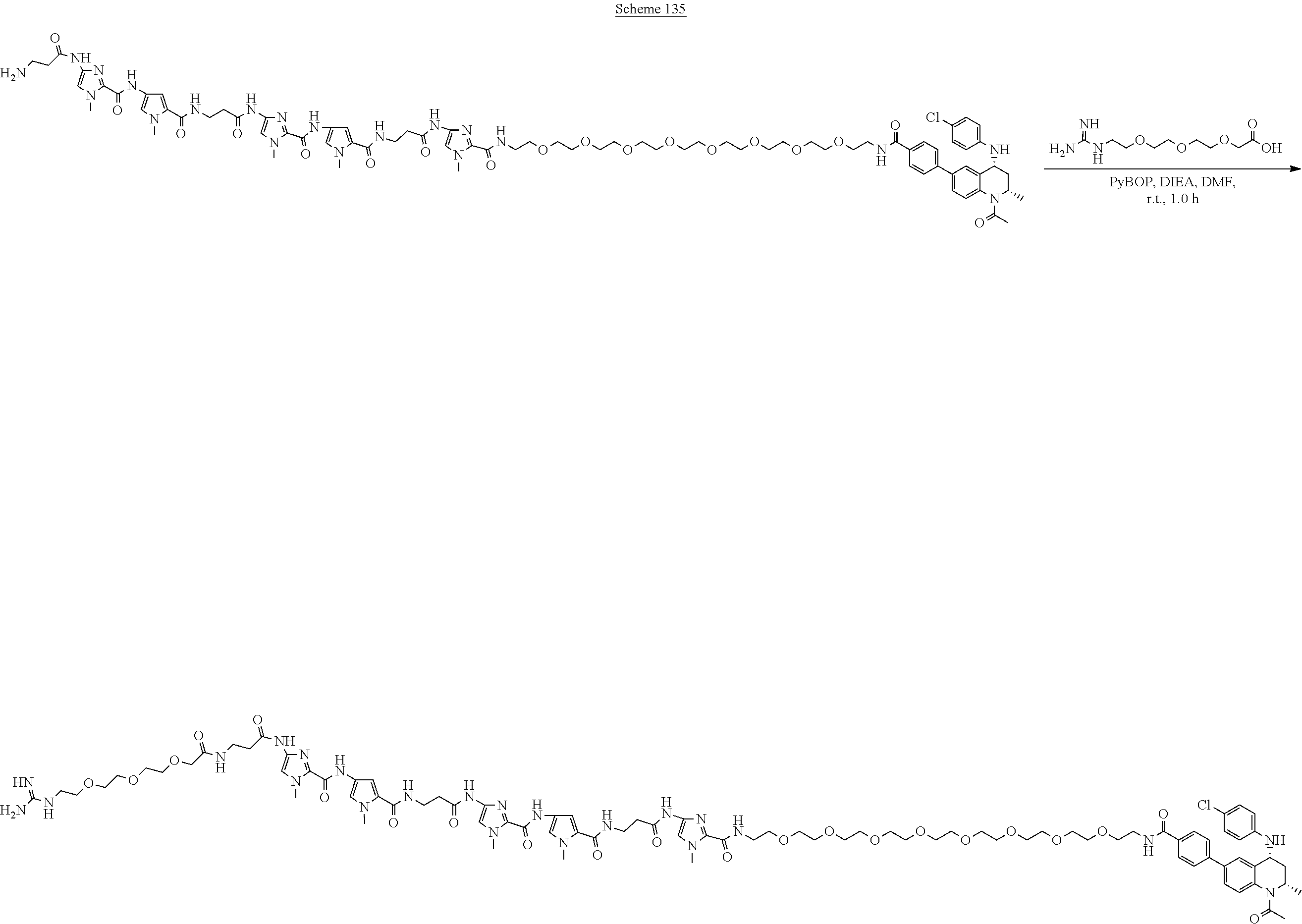 [SMILES: CC(=O)N1c2ccc(-c3ccc(C(=O)NCCOCCOCCOCCOCCOCCOCCOCCOCCNC(=O)c4nc(NC(=O)CCNC(=O)c5cc(NC(=O)c6nc(NC(=O)CCNC(=O)c7cc(NC(=O)c8nc(NC(=O)CCN)cn8C)cn7C)cn6C)cn5C)cn4C)cc3)cc2[C@H](Nc2ccc(Cl)cc2)C[C@@H]1C ; N=C(N)NCCOCCOCCOCC(=O)O ; CC(=O)N1c2ccc(-c3ccc(C(=O)NCCOCCOCCOCCOCCOCCOCCOCCOCCNC(=O)c4nc(NC(=O)CCNC(=O)c5cc(NC(=O)c6nc(NC(=O)CCNC(=O)c7cc(NC(=O)c8nc(NC(=O)CCNC(=O)COCCOCCOCCNC(=N)N)cn8C)cn7C)cn6C)cn5C)cn4C)cc3)cc2[C@H](Nc2ccc(Cl)cc2)C[C@@H]1C]

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate, but the product was purified by Prep-HPLC. 50.00 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-([4-[4-(3-aminopropanamido)-1-methylimidazole-2-amido]-1-methylpyrrol-2-yl]formamido)propanamido]-1-methylimidazole-2-carboxamide was used, 1.90 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-[(4-[4-[3-(2-[2-[2-(2-carbamimidamidoethoxy)ethoxy]ethoxy]acetamido)propanamido]-1-methylimidazole-2-amido]-1-methylpyrrol-2-yl)formamido]propanamido]-1-methylimidazole-2-carboxamide was obtained as white solid (3.30% yield). HRMS: mass calcd. For C₈₈H₁₂₀ClN₂₃O₂₂: 1885.8667, found: 1886.8723 [M+H]⁺.

### N-[5-([2-[(2-[[26-([4-1(2S,4R)-1-acetyl-4-[(4-chlorophenyl)aminol-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-1-methyl-4-[3-([1-methyl-4-[1-methyl-4-(3-[2-[2-(2-[2-[4-(piperidin-4-ylmethyl)piperazin-1-yl]ethoxy]ethoxy)ethoxy]acetamido]propanamido)imidazole-2-amido]pyrrol-2-yl]formamido)propanamido]imidazole-2-carboxamide (Compound 88)

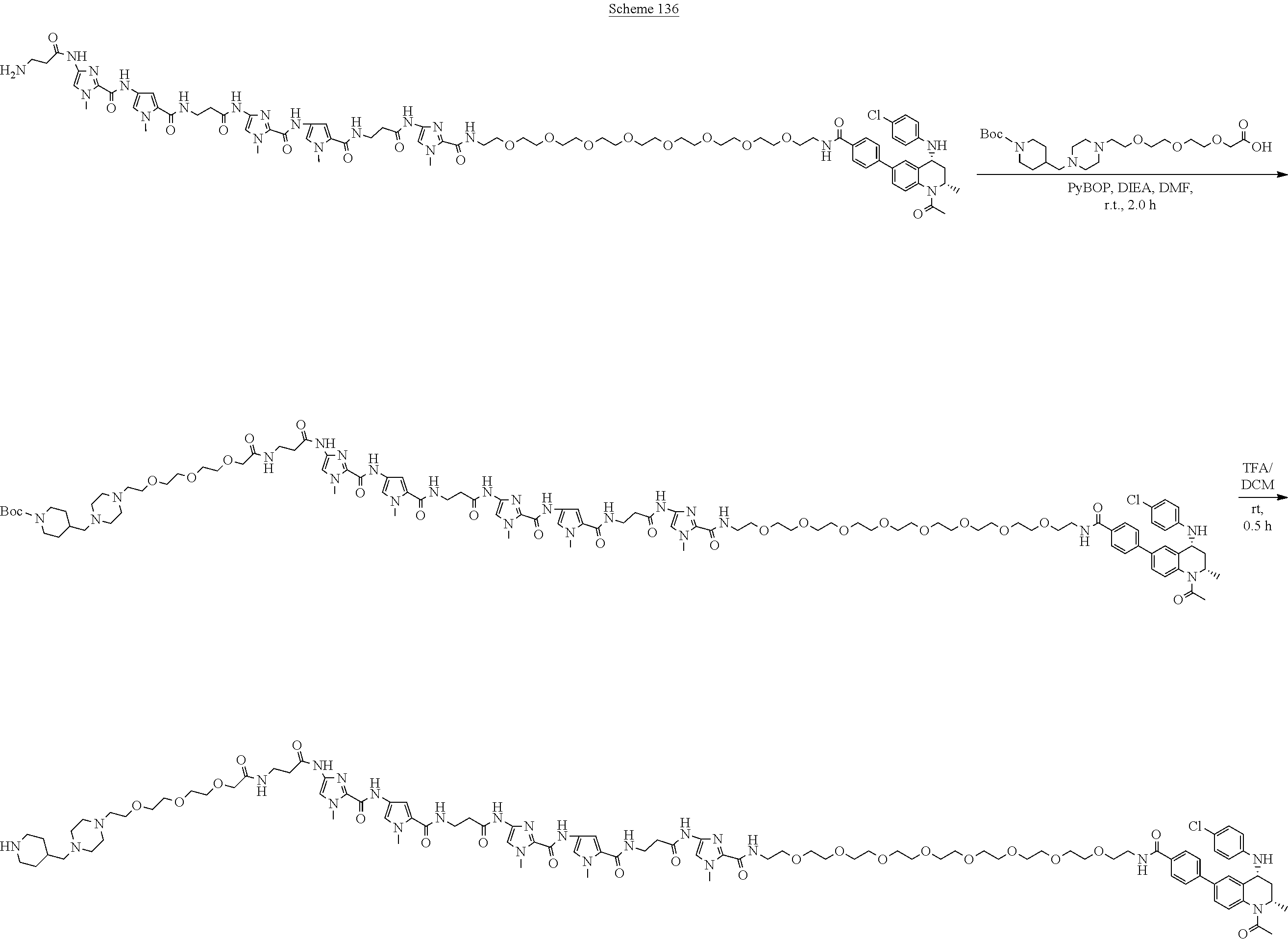

### Step 1: Synthesis of tert-butyl 4-([4-[2-(2-[2-[([2-[(2-[[5-([2-[(2-[[5-([2-j(2-[[26-([4-[(2S,4R)-1-acetyl-4-[(4-chlorophenyl)aminol-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-methylpyrrol-3-yl]carbamoyl]-1-methylimidazol-4-yl)carbamoyl]ethyl]carbamoyl)methoxy]ethoxy]ethoxy)ethyl]piperazin-1-yl]methyl)piperidine-1-carboxylate

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate. 50.00 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-([4-[4-(3-aminopropanamido)-1-methylimidazole-2-amido]-1-methylpyrrol-2-yl]formamido)propanamido]-1-methylimidazole-2-carboxamide was used, 30.00 mg of tert-butyl 4-([4-[2-(2-[2-[([2-[(2-[[5-([2-[(2-[[5-([2-[(2-[[26-([4-[(2S,4R)-1-acetyl-4-[(4- chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)methoxy]ethoxy]ethoxy)ethyl]piperazin-1-yl]methyl)piperidine-1-carboxylate was obtained as white solid (44.70% yield) LC/MS: mass calcd. For C₁₀₂H₁₄₄ClN₂₃O₂₄: 2110.04 found: 1057.05 [M/2+H]⁺.

### Step 2: Synthesis of N-[5-([2-[(2-[[26-([4-(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-1-methyl-4-[3-([1-methyl-4-[1-methyl-4-(3-[2-[2-[2-[2-[4-(piperidin-4-ylmethyl)piperazin-1-yl]ethoxy]ethoxy)ethoxy]acetamido]propanamido)imidazole-2-amido]pyrrol-2-yl]formamido)propanamido]imidazole-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide, but the product was purified by Prep-HPLC. 30.00 mg of tert-butyl 4-([4-[2-(2-[2-[([2-[(2-[[5-([2-[(2-[[5-([2-[(2-[[26-([4-[(2S,4R)-1-acetyl-4-[(4- chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)methoxy]ethoxy]ethoxy)ethyl]piperazin-1-yl]methyl)piperidine-1-carboxylate was used, 5.70 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-1-methyl-4-[3-([1-methyl-4-[1-methyl-4-(3-[2-[2-(2-[2-[4-(piperidin-4-ylmethyl)piperazin-1-yl]ethoxy]ethoxy)ethoxy]acetamido]propanamido)imidazole-2-amido]pyrrol-2-yl]formamido)propanamido]imidazole-2-carboxamide was obtained as white solid (1.63% yield). HRMS: mass calcd. For C₉₇H₁₃₆ClN₃O₂₂: 2009.9919 found: 2010.9990 [M+H]⁺.

### N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-[(4-4-[3-(47-amino-3,6,9,12,15,18,21,24,27,30,33,36,39,42,45-pentadecaoxaheptatetracontanamido)propanamido]-1-methylimidazole-2-amido]-1-methylpyrrol-2-yl)formamido]propanamido]-1-methylimidazole-2-carboxamide (Compound 89)

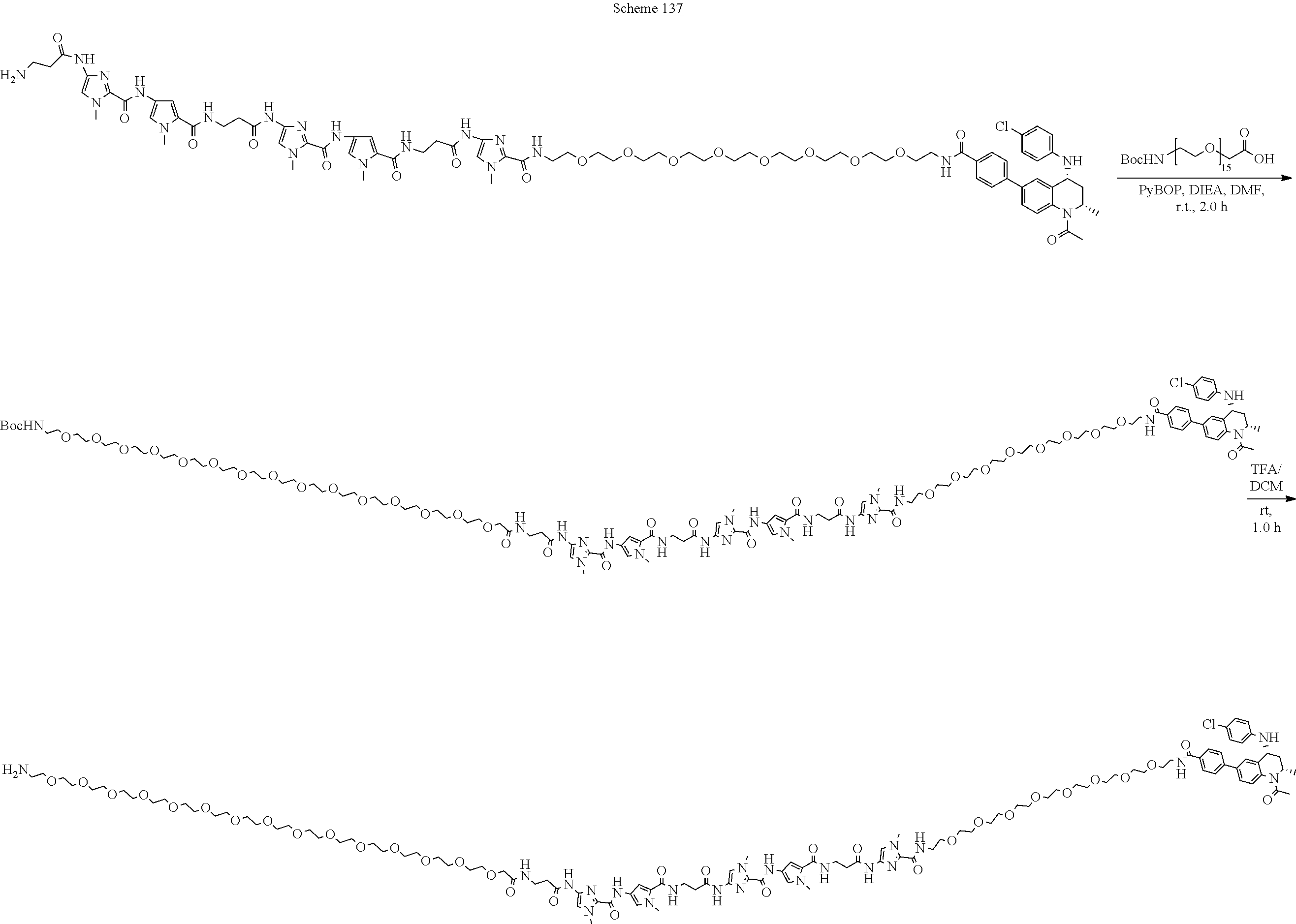

### Step 1: Synthesis of tert-butyl 4-([4-[2-(2-[2-[([2-[(2-[[5-([2-[(2-[[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)methoxy]ethoxy]ethoxy)ethyl]piperazin-1-yl]methyl)piperidine-1-carboxylate

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate and the reaction time was 2 h. 50.00 mg of N-[5-([2-[2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-([4-[4-(3-aminopropanamido)-1-methylimidazole-2-amido]-1-methylpyrrol-2-yl]formamido)propanamido]-1-methylimidazole-2-carboxamide was used, 30.00 mg of tert-butyl 4-([4-[2-(2-[2-[([2-[(2-[[5-([2-1(2-[[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethylcarbamoyl)methoxy]ethoxy]ethoxy)ethyl]piperazin-1-yl]methyl)piperidine-1-carboxylate was obtained as white solid (44.70% yield). LC/MS: mass calcd. C₁₁₆H₁₇₄ClN₂₁O₃₆: 2472.21 found: 1237.90 [M/2+H]⁺.

### Step 2: Synthesis of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-[(4-[4-[3-(47-amino-3,6,9,12,15,18,21,24,27,30,33,36,39,42,45-pentadecaoxaheptatetracontanamido)propanamido]-1-methylimidazole-2-amido]-1-methylpyrrol-2-yl)formamido]propanamido]-1-methylimidazole-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide, but the product was purified by Prep-HPLC. 30.00 mg of tert-butyl N-[1-([2-[(2-[[5-([2-[(2-[[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-2,5,8,11,14,17,20,23,26,29,32,35,38,41,44-pentadecaoxahexatetracontan-46-yl]carbamate was used, 0.60 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-[3-[(4-[4-[3-(47-amino-3,6,9,12,15,18,21,24,27,30,33,36,39,42,45-pentadecaoxaheptatetracontanamido)propanamido]-1-methylimidazole-2-amido]-1-methylpyrrol-2-yl)formamido]propanamido]-1-methylimidazole-2-carboxamide was obtained as white solid (2.08% yield). HRMS: mass calcd. For C₁₁₁H₁₆₆ClN₂₁O₃₄: 2372.1595 found: 2373.1733 [M+H]⁺.

### N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-(3-[[4-(4-[3-[(2S)-2-(2S)-2-amino-5-carbamimidamidopentanamido]-5-carbamimidamidopentanamido]propanamido]-1-methylimidazole-2-amido)-1-methylpyrrol-2-yl]formamido]propanamido)-1-methylimidazole-2-carboxamide (Compound 90)

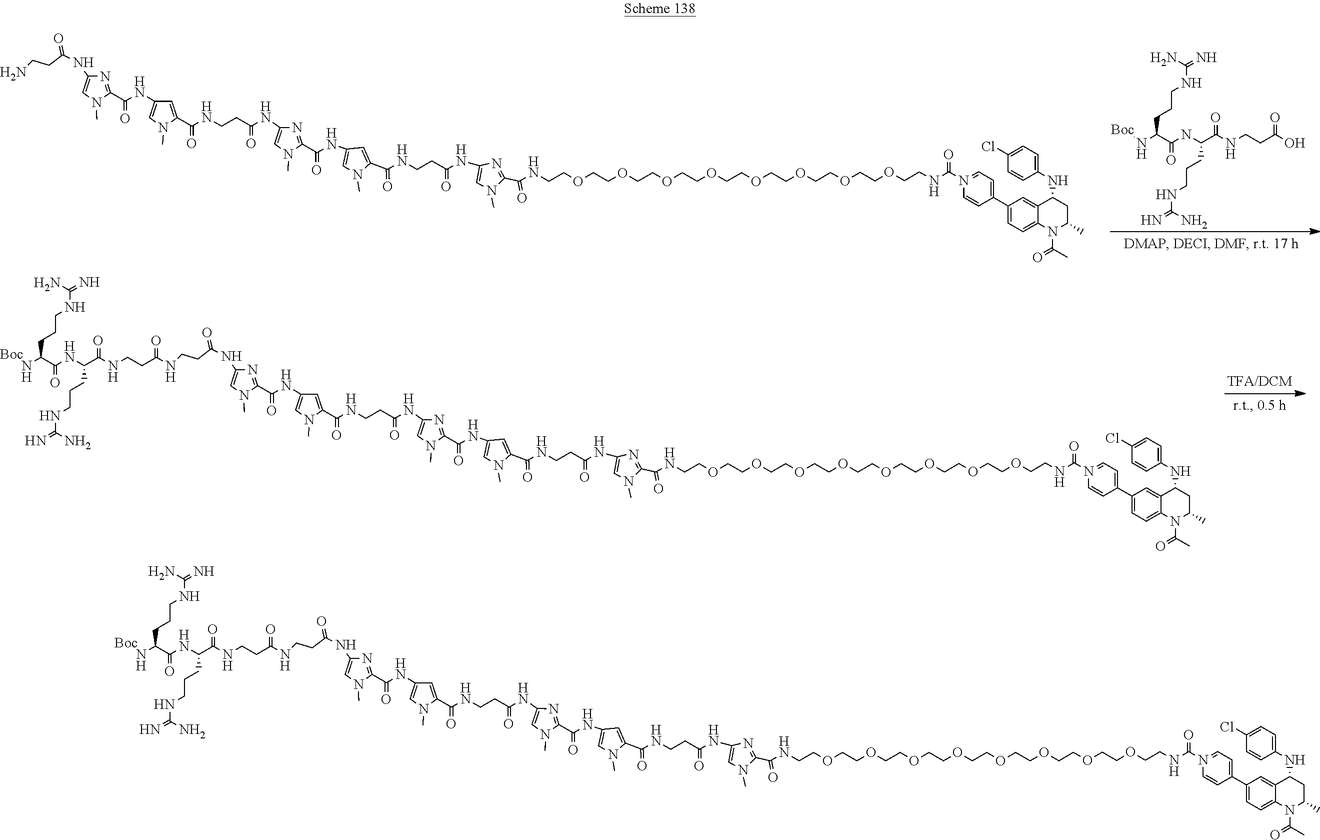

### Step 1: Synthesis of tert-butyl N-[(1S)-1-[[(1S)-1-([2-[(2-[[5-([2-[(2-[[5-([2-[(2-[[26-([[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-4-carbamimidamidobutyl]carbamoyl]-4-carbamimidamidobutyl]carbamate

The procedure was the same as N-[5-[(2-[[2-([2-[(3-[[3-(1-[4-[7-(3,5-dimethyl-1,2-oxazol-4-yl)-8-methoxy-1-[(2R)-1-methoxypropan-2-yl]imidazo[4,5-c]quinolin-2-yl]piperidin-1-yl]-1-oxo-3,6,9,12,15-pentaoxaoctadecan-18-amido)propyl](methyl)amino]propyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide and the product was purified by reverse phase column. 60.00 mg of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl-1-methylimidazol-4-yl)carbamoyl]ethyl]carbamoyl)-1-methylpyrrol-3-yl]-4-[3-([4-[4-(3-aminopropanamido)-1-methylimidazole-2-amido]-1-methylpyrrol-2-yl]formamido)propanamido]-1-methylimidazole-2-carboxamide was used. 50.00 mg of tert-butyl N-[(1S)-1-[[(1S)-]-([2-[(2-[[5-([2-[(2-[[5-([2-[(2-[[26-([4-[(2S,4R)-1-acetyl-4-[(4- chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-4-carbamoyl)-4-carbamimidamidobutyl]carbamoyl]-4-carbamimidamidobutyl]carbamate was obtained as white solid (56.71% yield). LC/MS: mass calcd. For C₉₆H₁₃₅ClN₂₈O₂₂: 2067.00 found: 1035.15 [M/2+H]⁺.

### Step 2: Synthesis of N-[5-([2-[(2-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-(3-[[4-(4-[3-1(2S)-2-[2S)-2-amino-5-carbamimidamidopentanamido]-5-carbamimidamidopentanamido]propanamido]-1-methylimidazole-2-amido)-1-methylpyrrol-2-yl]formamido]propanamido)-1-methylimidazole-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide, but the product was purified by Prep-HPLC. 50.00 mg of tert-butyl N-[(1S)-1-[1[(S)-1-([2-[(2-[[5-([2-[(2-[[5-([2-[(2-[[26-([4-[(2S,4R)-1-acetyl-4-[(4- chlorophenyl)aminol-2-methyl-3,4-dihydro-2-[1-quinolin-6-yl]phenyl]formamido)-3,6,9,12,15,18,21,24-octaoxahexacosan-1-yl]carbamoyl]-1-methylimidazol-4-yl)carbamoyl]ethyl]carbamyl)-1-methylpyrrol-3-yl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-4-carbamimidamidobutyl]carbamoyl]-4-carbamimidamidobutyl]carbamate was used, 4.40 mg of N-[5-([2-[(2-[[26-([4-[(2S,4R)-1-acetyl-4-[(4-chlorophenyl)amino]-2-methyl-3,4-dihydro-2H-quinolin-6-yl]phenylformamido)-3,6,9,12,15,18,21,24-octaoxahexacosan-1-yl]carbamoyl]-1-methylimidazol-4-yl)carbamoyl]ethyl]carbamoyl)-1-methylpyrrol-3-yl]-4-(3-[[4-(4-[3-[(2S)-2-[(2S)-2-amino-5-carbamimidamidopentanamido]-5-carbamimidamidopentanamido]propanamido]-1-methylimidazole-2-amido)-1-methylpyrrol-2-yl]formamido]propanamido)-1-methylimidazole-2-carboxamide was obtained as white solid (9.08% yield). HRMS: mass calcd. For C₉₁H₁₂₇ClN₂₈O₂₀: 1966.9470 found: 1967.9456 [M+H]⁺.

### N-(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)-4-(3-[[4-(4-amino-1-methylimidazole-2-amido)-1-methylpyrrol-2-yl]formamido]propanamido)-1-methylimidazole-2-carboxamide (Compound 92)

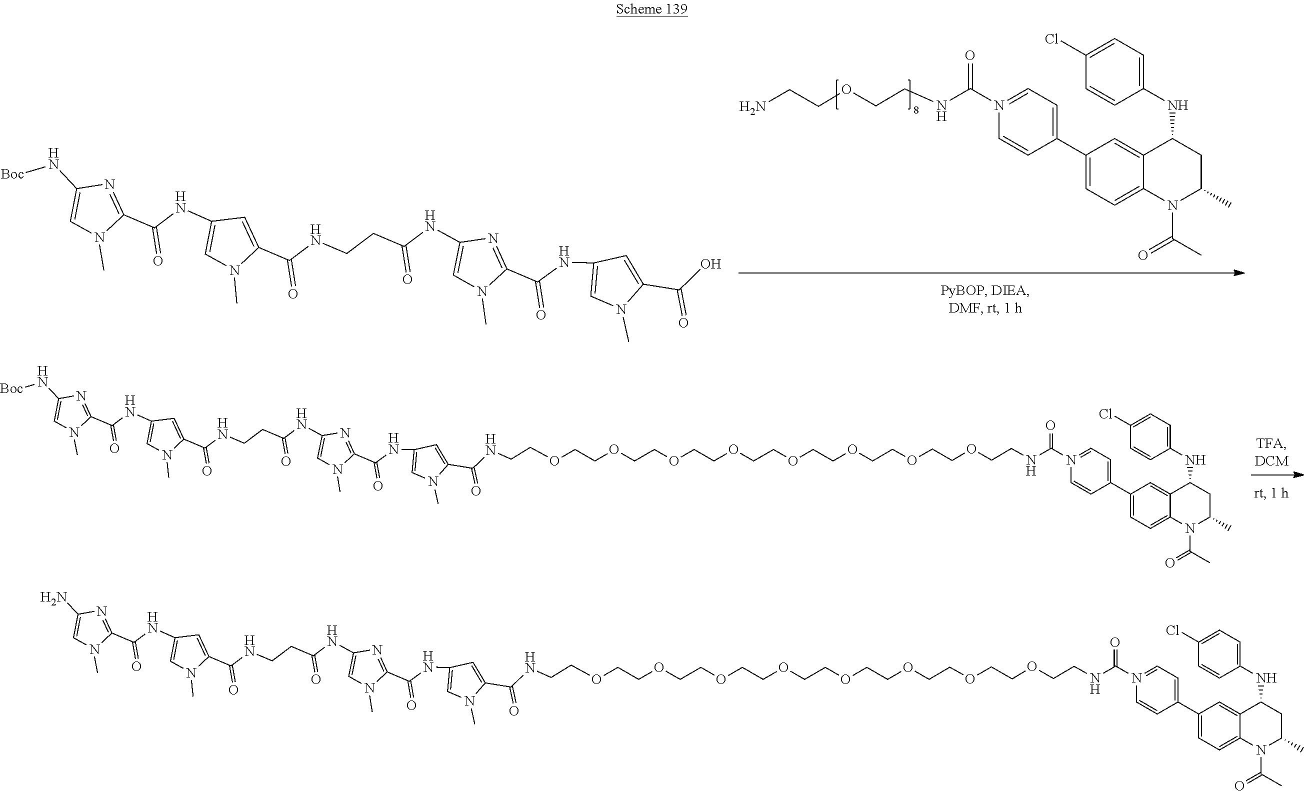

### Step 1: Synthesis of tert-butyl N-[2-[(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamate

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate. 50.00 mg of 4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrole-2-carboxylic acid was used, 50.00 mg of tert-butyl N-[2-[(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamate was obtained as light yellow solid (43.30% yield). LCMS: mass calcd. For C₇₃H₉₆ClN₁₅O₁₇: 1489.68, found: 746.30 [M/2+H]⁺.

### Step 2: Synthesis of N-(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)-4-(3-[[4-(4-amino-1-methylimidazole-2-amido)-1-methylpyrrol-2-yl]formamido]propanamido)-1-methylimidazole-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide, but the product was purified by Prep-HPLC. 50.00 mg of tert-butyl N-[2-[(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamate was used, 6.20 mg of N-(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)-4-(3-[[4-(4-amino-1-methylimidazole-2-amido)-1-methylpyrrol-2-yl]formamido]propanamido)-1-methylimidazole-2-carboxamide was obtained as white solid (12.72% yield). HRMS: mass calcd. For C₆₈H₈₈ClN₁₅O₁₅: 1389.6273, found: 1390.6333 [M+H]⁺.

### N-(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)-4-[3-([4-[4-(47-amino-3,6,9,12,15,18,21,24,27,30,33,36,39,42,45-pentadecaoxaheptatetracontanamido)-1-methylimidazole-2-amido]-1-methylpyrrol-2-yl]formamido)propanamido]-1-methylimidazole-2-carboxamide (Compound 96)

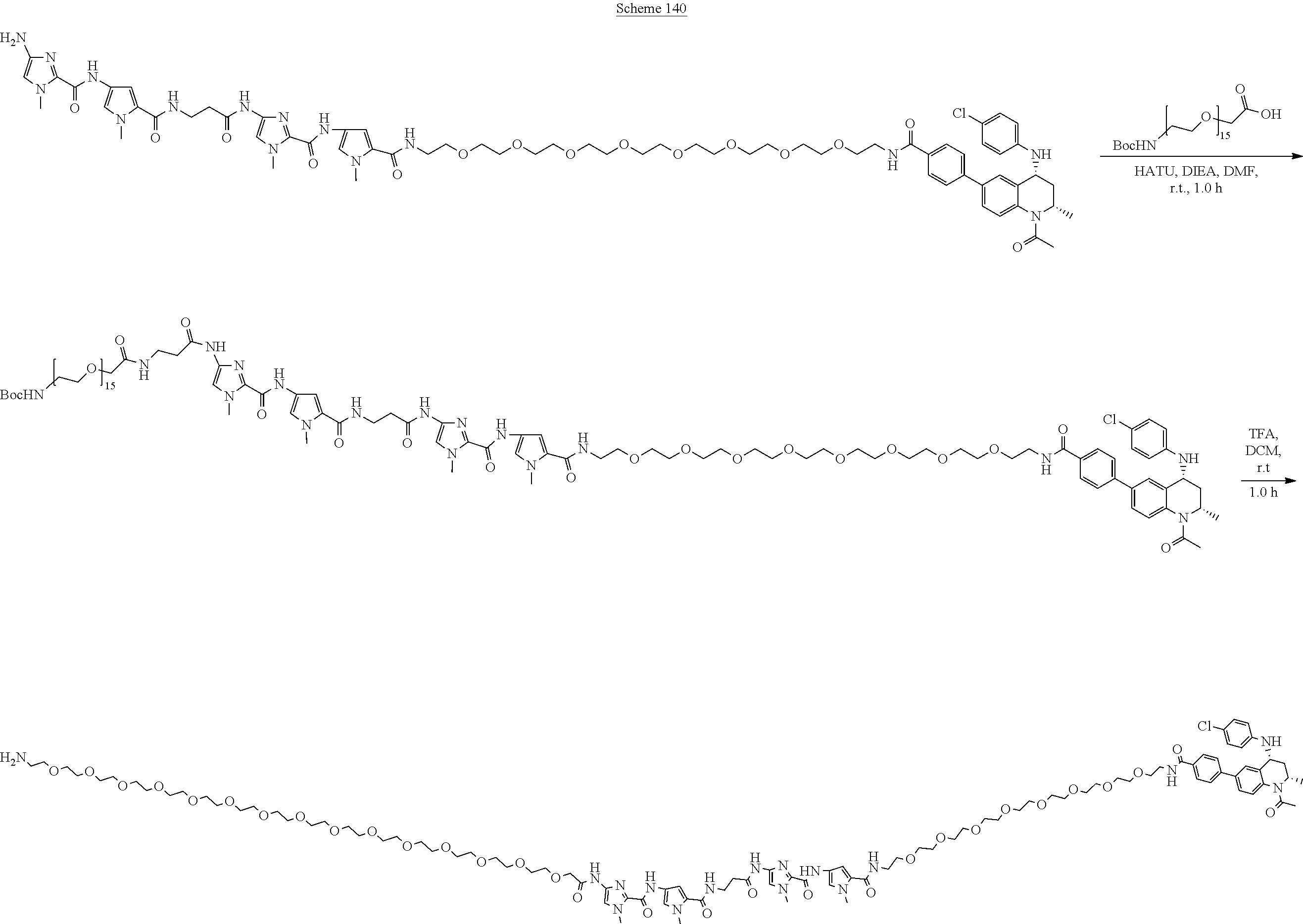

### Step 1: Synthesis of tert-butyl N-[1-([2-[(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamoyl)-2,5,8,11,14,17,20,23,26,29,32,35,38,41,44-pentadecaoxahexatetracontan-46-yl]carbamate

The procedure was the same as (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-2,24,34-trioxo-6,9,12,15,18,21-hexaoxa-3,25,29,33-tetraazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide and the product was purified by reverse phase column. 60.00 mg of N-(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)-4-(3-[[4-(4-amino-1-methylimidazole-2-amido)-1-methylpyrrol-2-yl]formamido]propanamido)-1-methylimidazole-2-carboxamide was used, 45.00 mg of tert-butyl N-[1-([2-[(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamoyl)-2,5,8,11,14,17,20,23,26,29,32,35,38,41,44-pentadecaoxahexatetracontan-46-yl]carbamate was obtained as yellow oil (42.50% yield). LCMS: mass calcd. For C₁₀₅H₁₅₉ClN₁₆O₃₃: 2207.09, found: 1127.65 [M/2+Na]⁺.

### Step 2: Synthesis of N-(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)-4-[3-([4-[4-(47-amino-3,6,9,12,15,18,21,24,27,30,33,36,39,42,45-pentadecaoxaheptatetracontanamido)-1-methylimidazole-2-amido]-1-methylpyrrol-2-yl]formamido)propanamido]-1-methylimidazole-2-carboxamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide, but the product was purified by Prep-HPLC. 45.00 mg of tert-butyl N-[1-([2-[(5-[[2-([2-[(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)carbamoyl]-1-methylimidazol-4-yl]carbamoyl)ethyl]carbamoyl]-1-methylpyrrol-3-yl)carbamoyl]-1-methylimidazol-4-yl]carbamoyl)-2,5,8,11,14,17,20,23,26,29,32,35,38,41,44-pentadecaoxahexatetracontan-46-yl]carbamate was used, 21.50 mg of N-(5-[[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamoyl]-1-methylpyrrol-3-yl)-4-[3-([4-[4-(47-amino-3,6,9,12,15,18,21,24,27,30,33,36,39,42,45-pentadecaoxaheptatetracontanamido)-1-methylimidazole-2-amido]-1-methylpyrrol-2-yl]formamido)propanamido]-1-methylimidazole-2-carboxamide was obtained as white solid (47.59% yield). HRMS: mass calcd. For C₁₀₀H₁₅₁ClN₁₆O₃₁: 2107.0420, found: 2108.0398 [M+H]⁺.

### (S)—N-(5-((3-((2-((1-((5-(2-(4-(4-chlorophenyl)-2,3,9-trimethyl-6H-thieno[3,2-f][1,2,4]triazolo[4,3-a][1,4]diazepin-6-yl)acetamido)pyridin-2-yl)oxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 102)

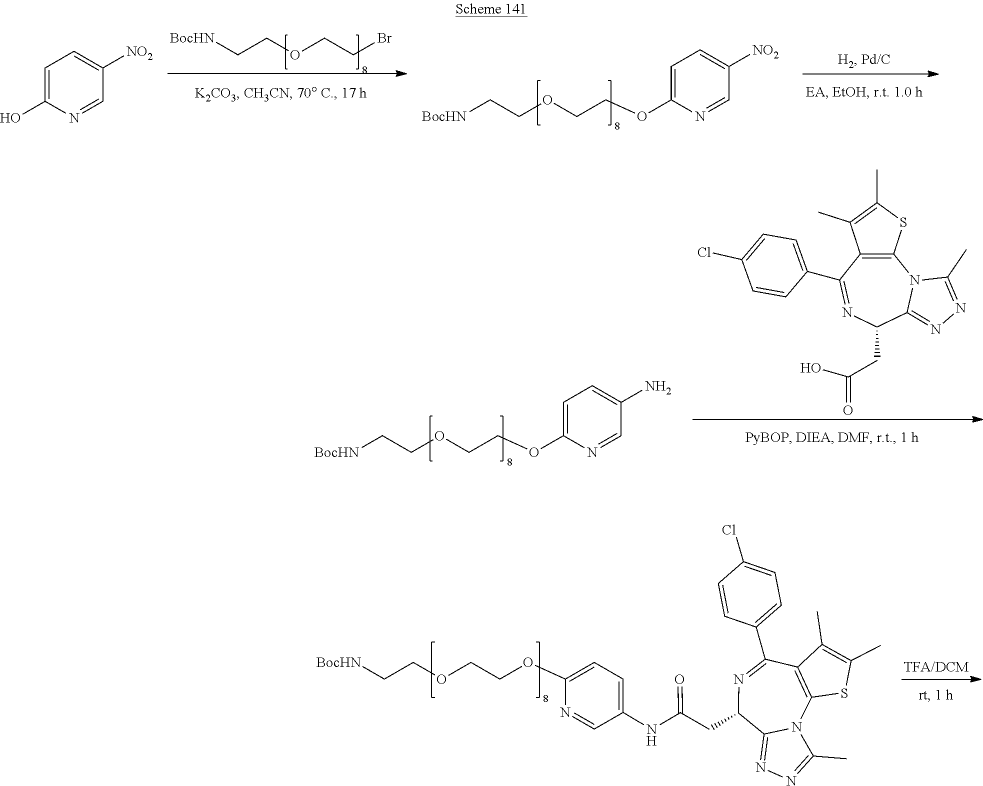

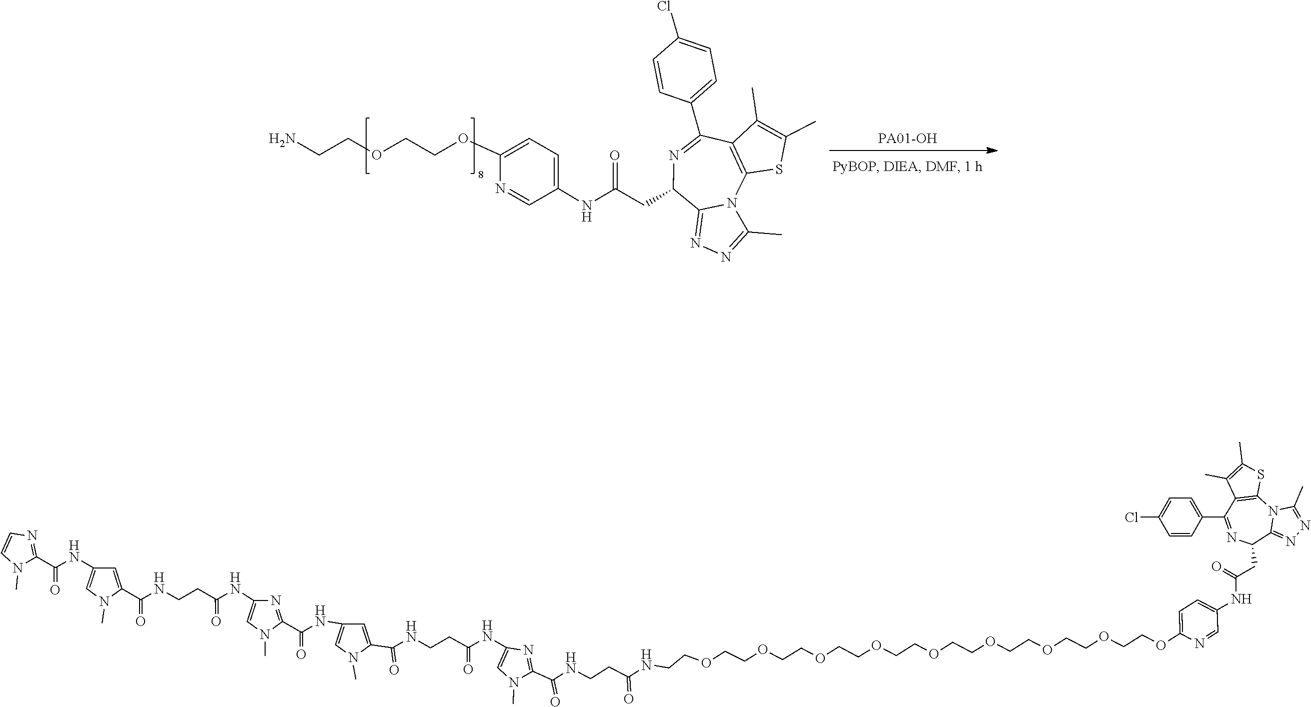

### Step 1: Synthesis of tert-butyl N-{26-[(5-nitropyridin-2-yl)oxy]-3,6,9,12,15,18,21,24-octaoxahexacosan-1-yl}carbamate

To a solution of 5-nitropyridin-2-ol (60.00 mg, 0.43 mmol, 1.00 equiv) and tert-butyl N-(26-bromo-3,6,9,12,15,18,21,24-octaoxahexacosan-1-yl)carbamate (296.29 mg, 0.51 mmol, 1.20 equiv) in CH₃CN (2.00 mL) was added K₂CO₃ (178.86 mg, 1.28 mmol, 3.00 equiv) at room temperature. The resulting mixture was stirred for 17 h at 70 degrees C. The solid was filtered out and washed by CH₃CN (10 mL). The filtrate was concentrated. The residue was purified by TLC-Plate with DCM:MeOH=15:1 to afford tert-butyl N-{26-[(5-nitropyridin-2-yl)oxy]-3,6,9,12,15,18,21,24-octaoxahexacosan-1-yl}carbamate (50.00 mg, 18.37%) as colorless oil. LC/MS: mass calcd. For C₂₈H₄₉N₃O₁₃: 635.32, found: 658.30 [M+Na]⁺. ¹H NMR (400 MHz, CDCl3) δ: 8.98 (s, 1H), 8.28 (d, J=8.8 Hz, 1H), 6.81 (d, J=7.2 Hz, 1H), 5.28 (brs, 1H), 4.51-4.53 (t, J=4.0 Hz, 2H), 3.79-3.81 (t, J=4.0 Hz, 2H), 3.61-3.66 (m, 28H), 3.53 (t, J=4.0 Hz, 2H), 3.23-3.40 (m, 2H), 1.37 (s, 9H).

### Step 2: Synthesis of tert-butyl N-[26-[(5-aminopyridin-2-yl)oxy]-3,6,9,12,15,18,21,24-octaoxahexacosan-1-yl]carbamate

Tert-butyl N-[26-[(5-nitropyridin-2-yl)oxy]-3,6,9,12,15,18,21,24-octaoxahexacosan-1-yl]carbamate (130.00 mg, 0.20 mmol, 1.00 equiv) was dissolved in the mixture of EA (1.50 mL) and EtOH (1.50 mL). Pd/C (13.00 mg, 10% w/w) was added and the mixture was stirred for 1 h at room temperature under H₂ atmosphere. The resulting mixture was filtered, the filter cake was washed with EA (3×2 mL). The filtrate was concentrated under reduced pressure to afford tert-butyl N-[26-[(5-aminopyridin-2-yl)oxy]-3,6,9,12,15,18,21,24-octaoxahexacosan-1-yl]carbamate (80.00 mg, 64.58%) as white oil. LC/MS: mass calcd. For C₂₈H₅₁N₃O₁₁: 605.35, found: 606.30 [M+H]⁺.

### Step 3: Synthesis of tert-butyl (S)-(26-((5-(2-(4-(4-chlorophenyl)-2,3,9-trimethyl-6H-thieno[3,2-f][1,2,4]triazolo[4,3-a][1,4]diazepin-6-yl)acetamido)pyridin-2-yl)oxy)-3,6,9,12,15,18,21,24-octaoxahexacosyl)carbamate

(S)-2-(4-(4-chlorophenyl)-2,3,9-trimethyl-6H-thieno[3,2-f][1,2,4]triazolo[4,3-a][1,4]diazepin-6-yl)acetic acid (52.95 mg, 0.13 mmol, 1.00 equiv) was dissolved in DMF (2.00 mL). PyBOP (103.09 mg, 0.20 mmol, 1.50 equiv), tert-butyl N-[26-[(5-aminopyridin-2-yl)oxy]-3,6,9,12,15,18,21,24-octaoxahexacosan-1-yl]carbamate (80.00 mg, 0.13 mmol, 1.00 equiv) and DIEA (51.21 mg, 0.40 mmol, 3.00 equiv) were added in turn to the solution at 0 degrees C. The mixture was allowed to warm to room temperature and stirred for 1 h. The mixture was purified by reverse flash chromatography with the following conditions: column, C18 column; mobile phase, ACN in water (0.05% NH₄HCO₃), 45% to 55% gradient in 20 min; detector, UV 254 nm and 220 nm. The fractions were combined and concentrated. Tert-butyl (S)-(26-((5-(2-(4-(4-chlorophenyl)-2,3,9-trimethyl-6H-thieno[3,2-f][1,2,4]triazolo[4,3-a][1,4]diazepin-6-yl)acetamido)pyridin-2-yl)oxy)-3,6,9,12,15,18,21,24-octaoxahexacosyl)carbamate (70.00 mg, 53.61%) was obtained as white oil. LC/MS: mass calcd. For C₄₇H₆₆ClN₇O₁₂S: 987.41, found: 1010.85 [M+Na]⁺.

### Step 4: Synthesis of (S)—N-(6-((26-amino-3,6,9,12,15,18,21,24-octaoxahexacosyl)oxy)pyridin-3-yl)-2-(4-(4-chlorophenyl)-2,3,9-trimethyl-6H-thieno[3,2-f][1,2,4]triazolo[4,3-a][1,4]diazepin-6-yl)acetamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 60.00 mg of tert-butyl (S)-(26-((5-(2-(4-(4-chlorophenyl)-2,3,9-trimethyl-6H-thieno[3,2-f][1,2,4]triazolo[4,3-a][1,4]diazepin-6-yl)acetamido)pyridin-2-yl)oxy)-3,6,9,12,15,18,21,24-octaoxahexacosyl)carbamate was used, 60.00 mg crude of (S)—N-(6-((26-amino-3,6,9,12,15,18,21,24-octaoxahexacosyl)oxy)pyridin-3-yl)-2-(4-(4-chlorophenyl)-2,3,9-trimethyl-6H-thieno[3,2-f][1,2,4]triazolo[4,3-a][1,4]diazepin-6-yl)acetamide was obtained as light yellow oil. LC/MS: mass calcd. for C₄₂H₅₈ClN₇O₁₀S: 887.37, found: 888.50 [M+H]⁺.

### Step 5: Synthesis of (S)—N-(5-((3-((2-((1-((5-(2-(4-(4-chlorophenyl)-2,3,9-trimethyl-6H-thieno[3,2-f][1,2,4]triazolo[4,3-a][1,4]diazepin-6-yl)acetamido)pyridin-2-yl)oxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-((5-(2-(4-(4-chlorophenyl)-2,3,9-trimethyl-6H-thieno[3,2-f][1,2,4]triazolo[4,3-a][1,4]diazepin-6-yl)acetamido)pyridin-3-yl)oxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 60.00 mg of (S)—N-(6-((26-amino-3,6,9,12,15,18,21,24-octaoxahexacosyl)oxy)pyridin-3-yl)-2-(4-(4-chlorophenyl)-2,3,9-trimethyl-6H-thieno[3,2-f][1,2,4]triazolo[4,3-a][1,4]diazepin-6-yl)acetamide was used, 28.00 mg of (S)—N-(5-((3-((2-((1-((5-(2-(4-(4-chlorophenyl)-2,3,9-trimethyl-6H-thieno[3,2-f][1,2,4]triazolo[4,3-a][1,4]diazepin-6-yl)acetamido)pyridin-2-yl)oxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (23.66% yield). HRMS: mass calcd. For C₇₈H₉₉ClN₂₂O₁₈S: 1698.6917, found: 1699.7012 [M+H]⁺.

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-22-methyl-27-oxo-3,6,9,12,15-pentaoxa-18,22,26-triazanonacosan-29-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 4)

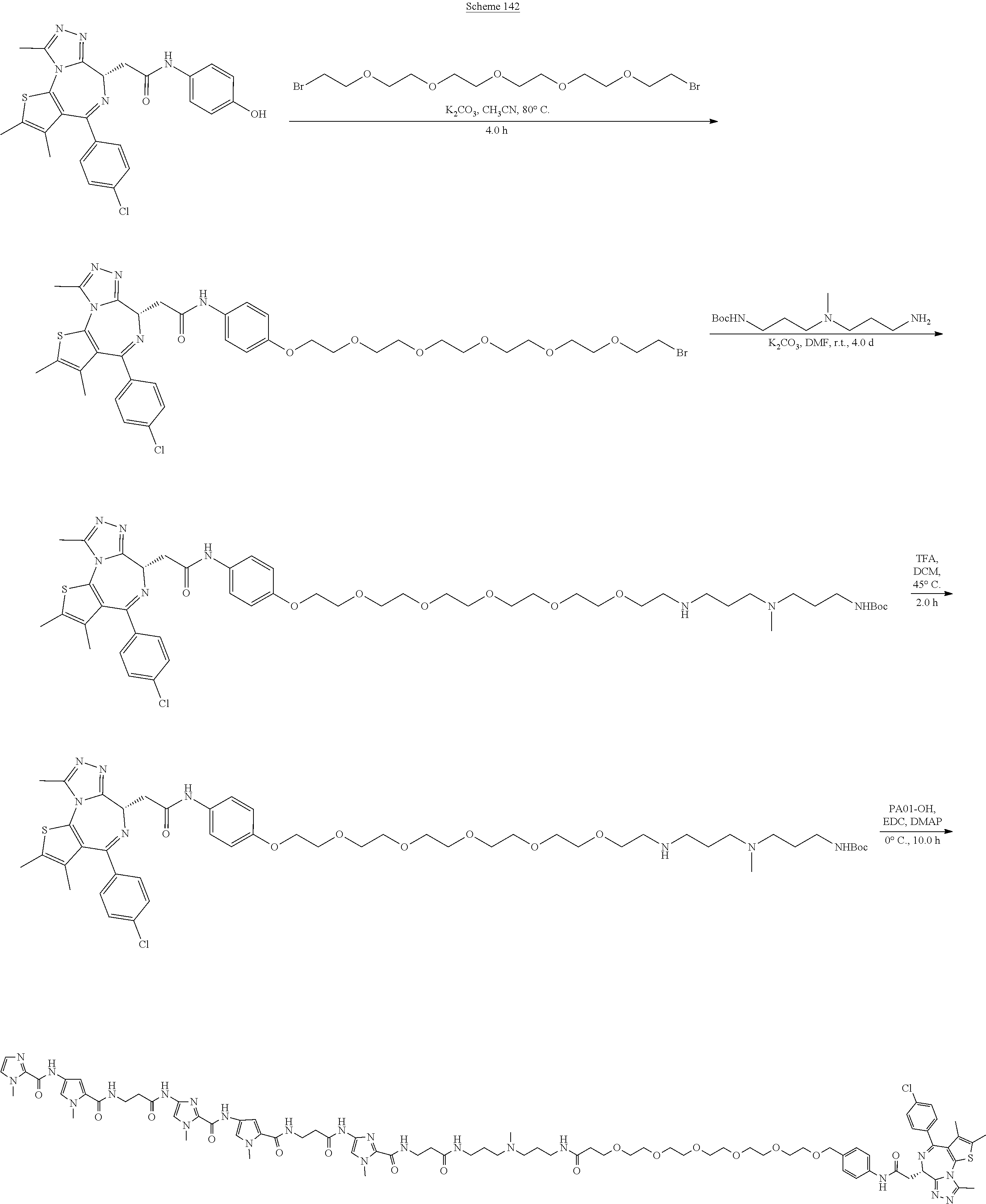

### Step 1: Synthesis of (S)—N-(4-((17-bromo-3,6,9,12,15-pentaoxaheptadecyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide

The procedure was the same as tert-butyl (S)-(26-(4-(2-(4-(4-chlorophenyl)-2,3,9-trimethyl-6H-thienol[3,2-f][1,2,4]triazolo[4,3-a][1,4]diazepin-6-yl)acetamido)phenoxy)-3,6,9,12,15,18,21,24-octaoxahexacosyl)carbamate. 100.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 140.00 mg of (S)—N-(4-((17-bromo-3,6,9,12,15-pentaoxaheptadecyl) oxy)phenyl)-2-(4-(4-chlorophenyl)-2,3,9-trimethyl-6H-thieno[3,2-f][1,2,4]triazolo[4,3-a][1,4]diazepin-6-yl)acetamide was obtained as yellow solid (82.40% yield). LCMS: mass calcd. For C₃₇H₄₅BrClN₅O₇S: 819.19, found: 820.35 [M+H]⁺.

### Step 2: Synthesis of tert-butyl (S)-(1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-22-methyl-3,6,9,12,15-pentaoxa-18,22-diazapentacosan-25-yl)carbamate

To a solution of (S)—N-(4-((17-bromo-3,6,9,12,15-pentaoxaheptadecyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide (120.00 mg, 0.15 mmol, 1.00 equiv) in DMF (3.00 mL) was added tert-butyl N-[3-[(3-aminopropyl)(methyl)amino]propyl]carbamate (39.54 mg, 0.16 mmol, 1.10 equiv) and K₂CO₃ (40.49 mg, 0.29 mmol, 2.00 equiv). The reaction mixture was stirred at room temperature for 4 days. The solid was filtered out and the filtration was purified by reverse flash chromatography with the following conditions: column, C18 silica gel; mobile phase, ACN in water (0.05% TFA), 5% to 50% gradient in 40 min; detector, UV 254 nm. The fractions were combined and concentrated to afford tert-butyl (S)-(1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-22-methyl-3,6,9,12,15-pentaoxa-18,22-diazapentacosan-25-yl)carbamate (120.00 mg, 83.28%) as colorless oil. LCMS: mass calcd. For C₄₉H₇₁ClN₈O₉S: 982.48, found: 983.55 [M+H]⁺.

### Step 3: Synthesis of (S)—N-(4-((25-amino-22-methyl-3,6,9,12,15-pentaoxa-18,22-diazapentacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide, but the reaction temperature was 45 degree C. 115.00 mg of tert-butyl (S)-(1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-22-methyl-3,6,9,12,15-pentaoxa-18,22-diazapentacosan-25-yl)carbamate was used, 115.00 mg crude of (S)—N-(4-((25-amino-22-methyl-3,6,9,12,15-pentaoxa-18,22-diazapentacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide was obtained as yellow oil. LCMS: mass calcd. For C₄₄H₆₃ClN₈O₇S: 882.42, found: 883.60 [M+H]⁺.

### Step 4: Synthesis of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-22-methyl-27-oxo-3,6,9,12,15-pentaoxa-18,22,26-triazanonacosan-29-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

To a solution of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (40.00 mg, 0.05 mmol, 1.00 equiv)) in DMF (3.00 mL) was added EDCI (13.86 mg, 0.07 mmol, 1.50 equiv), (S)—N-(4-((25-amino-22-methyl-3,6,9,12,15-pentaoxa-18,22-diazapentacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide (42.59 mg, 0.05 mmol, 1.00 equiv) and DMAP (14.72 mg, 0.12 mmol, 2.50 equiv) at 0 degrees C. Then the reaction was stirred at 0 degrees C. for 10 h. The crude reaction mixture was filtered and the filtration was purified by Prep-HPLC directly with the following conditions: Column: XSelect CSH Prep C18 OBD Column, 19*250 mm, 5 um; Mobile Phase A: Water(0.05% TFA), Mobile Phase B: ACN; Flow rate: 25 mL/min; Gradient: 15 B to 40 B in 15 min; 254 nm; RT: 13.92. The fractions were combined and lyophilized directly to afford (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-22-methyl-27-oxo-3,6,9,12,15-pentaoxa-18,22,26-triazanonacosan-29-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (22.2 mg, 26.19%) as white solid. HRMS: mass calcd. For C₈₀H₁₀₄ClN₂₃O₁₅S: 1693.7491, found: 1694.7559 [M+H]⁺.

### (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-24,34-dioxo-3,6,9,12,15,18,21-heptaoxa-25,29,33-triazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 14)

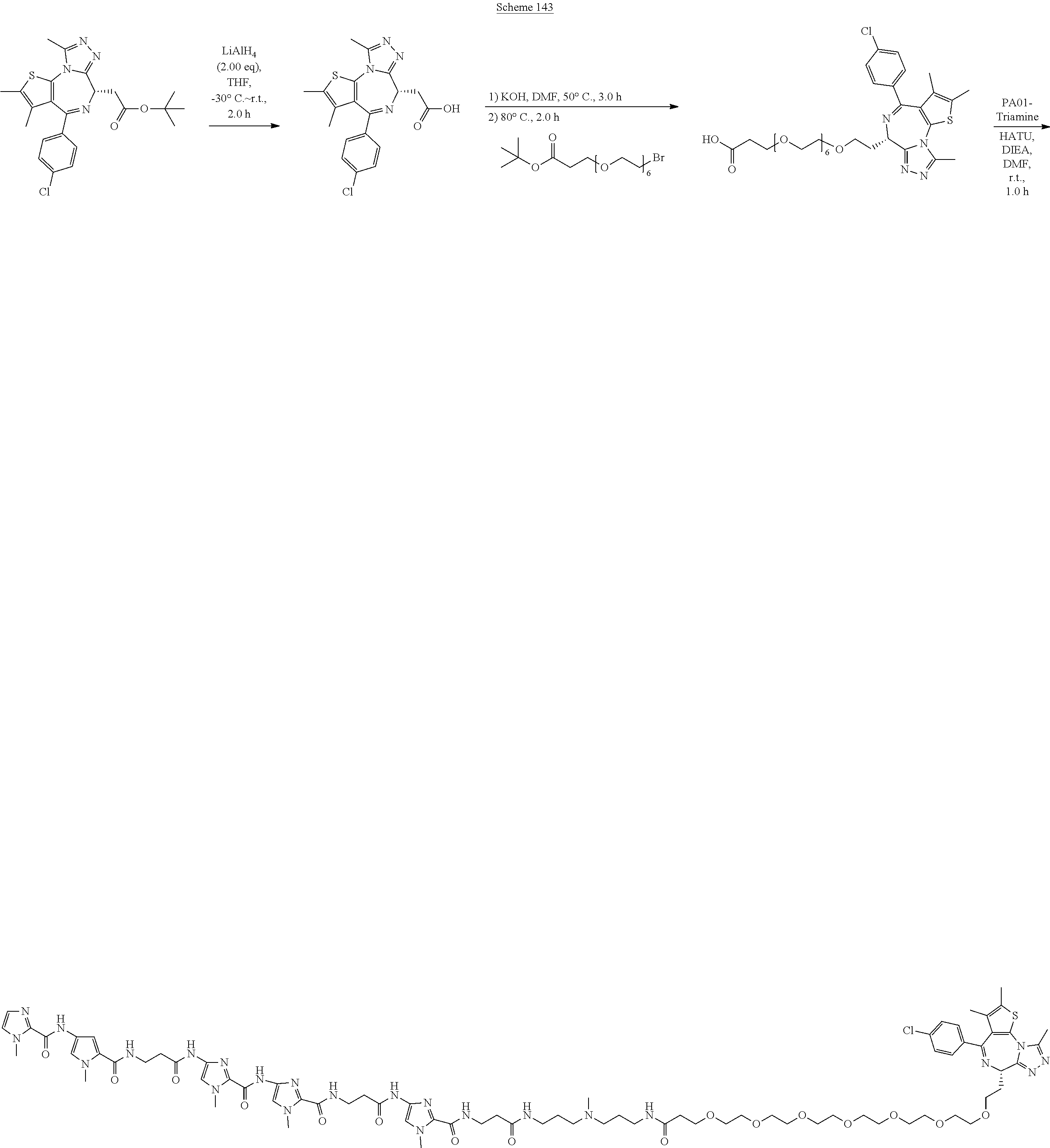

### Step 1: Synthesis of (S)-2-(4-(4-chlorophenyl)-2,3,9-trimethyl-6H-thieno[3,2-f][1,2,4]triazolo[4,3-a][1,4]diazepin-6-yl)ethan-1-ol

Into a 100 ml flask was added tert-butyl (S)-2-(4-(4-chlorophenyl)-2,3,9-trimethyl-6H-thieno[3,2-f][1,2,4]triazolo[4,3-a][1,4]diazepin-6-yl)acetate (300.00 mg, 0.66 mmol, 1.00 equiv), THF (8.00 mL), the reaction was stirred at −30 degrees C. and under N₂ atmosphere, then LiAlH₄ (1.0 M/THF, 0.79 mL, 0.79 mmol, 1.20 equiv) was added dropwise, the reaction was stirred at −30 degrees C. to room temperature for 2 h. The reaction was cooled to −30 degrees C., quenched with MeOH (10 mL), then concentrated, the residue was purified by TLC plate with PE:EA=1:1. This resulted in (S)-2-(4-(4-chlorophenyl)-2,3,9-trimethyl-6H-thieno[3,2-f][1,2,4]triazolo[4,3-a][1,4]diazepin-6-yl)ethan-1-ol (190.00 mg, 70.16%) as yellow solid. LC/MS: mass calcd. For C₁₉H₁₉ClN₄OS: 386.90, found: 387.15 [M+H]⁺.

### Step 2: Synthesis of (S)-1-(4-(4-chlorophenyl)-2,3,9-trimethyl-6H-thieno[3,2-f][1,2,4]triazolo[4,3-a][1,4]diazepin-6-yl)-3,6,9,12,15,18,21-heptaoxatetracosan-24-oic acid

Into a 25 ml flask was added (S)-2-(4-(4-chlorophenyl)-2,3,9-trimethyl-6H-thieno[3,2-f][1,2,4]triazolo[4,3-a][1,4]diazepin-6-yl)ethan-1-ol (220.00 mg, 0.57 mmol, 1.00 equiv), DMSO (8.00 mL), KOH (95.71 mg, 1.71 mmol, 3.00 equiv), the reaction was stirred at 50 degrees C. under N₂ atmosphere for 3 h, then tert-butyl 1-bromo-3,6,9,12,15,18-hexaoxahenicosan-21-oate (269.19 mg, 0.57 mmol, 1.00 equiv) was added and stirred at 80 degrees C. for 2 h, then the reaction was stirred at room temperature for 16 h. The solid was filtered out and the filtration was purified by reverse flash column with the following conditions: column, C18; mobile phase, ACN in water(0.05% TFA), 10% to 50% gradient in 60 min; detector, UV 254 nm. The fractions were combined and concentrated. This resulted in (S)-1-(4-(4-chlorophenyl)-2,3,9-trimethyl-6H-thieno[3,2-f][1,2,4]triazolo[4,3-a][1,4]diazepin-6-yl)-3,6,9,12,15,18,21-heptaoxatetracosan-24-oic acid (30.00 mg, 2.76%) as yellow oil. LC/MS: mass calcd. For C₃₄H₄₇ClN₄O₉S: 722.27, found: 723.20 [M+H]⁺.

### Step 3: Synthesis of (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-24,34-dioxo-3,6,9,12,15,18,21-heptaoxa-25,29,33-triazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-2,24,34-trioxo-6,9,12,15,18,21-hexaoxa-3,25,29,33-tetraazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 20.00 mg of (S)-1-(4-(4-chlorophenyl)-2,3,9-trimethyl-6H-thieno[3,2-f][1,2,4]triazolo[4,3-a][1,4]diazepin-6-yl)-3,6,9,12,15,18,21-heptaoxatetracosan-24-oic acid was used, 2.20 mg of (S)—N-(5-((3-((2-((1-(4-(4-chlorophenyl)-2,3,9-trimethyl-6H-thieno[3,2-f][1,2,4]triazolo[4,3-a][1,4]diazepin-6-yl)-29-methyl-24,34-dioxo-3,6,9,12,15,18,21-heptaoxa-25,29,33-triazahexatriacontan-36-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as yellow solid (4.43% yield). HRMS: mass calcd. For C₇₇H₁₀₅ClN₂₂O₁₆S: 1660.7488, found: 1661.7574 [M+H]⁺.

### (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenyl)-24-methyl-19,29-dioxo-4,7,10,13,16-pentaoxa-20,24,28-triazahentriacontan-31-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 60)

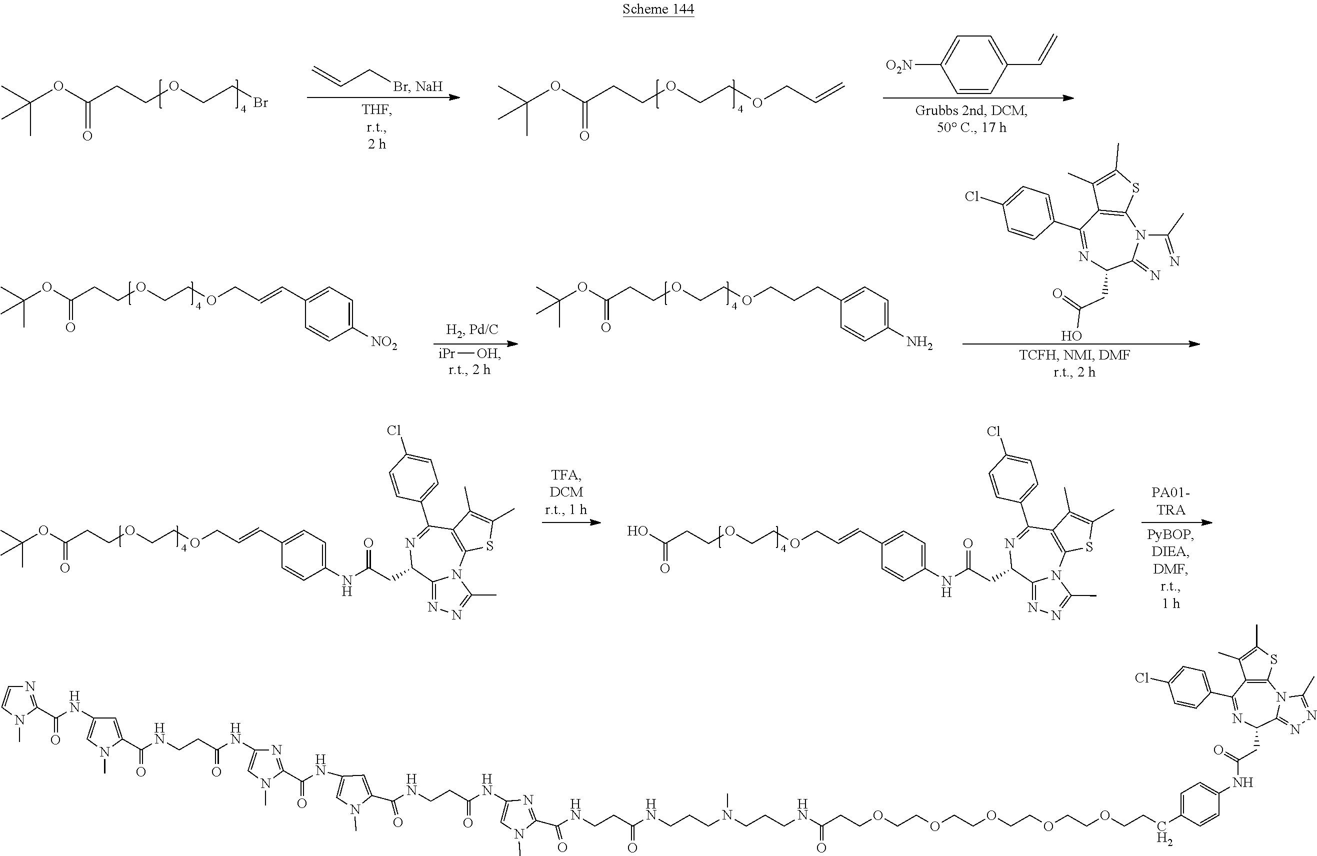

### Step 1: Synthesis of tert-butyl 4,7,10,13,16-pentaoxanonadec-18-enoate

To a solution of tert-butyl 1-hydroxy-3,6,9,12-tetraoxapentadecan-15-oate (1.00 g, 3.10 mmol, 1.00 equiv) in dry THF (30.00 mL) was added NaH (60%) (149.00 mg, 3.72 mmol, 1.20 equiv) at 0 degrees C. The mixture was stirred at this temperature for 10 mins. Then 3-bromoprop-1-ene (450.00 mg, 3.72 mmol, 1.20 equiv) was added to the above solution. The reaction solution was stirred at room temperature for 2 h. The reaction was quenched with aq. NH₄Cl (30 mL) at 0 degrees C. The resulting mixture was extracted with EA (3×50 mL). The combined organic layers were washed with brine (1×50 mL), dried over anhydrous Na₂SO₄. After filtration, the filtrate was concentrated under reduced pressure. Tert-butyl 4,7,10,13,16-pentaoxanonadec-18-enoate (620.00 mg, 55.15%) was obtained as yellow oil. LC/MS: mass calcd. For C₁₈H₃₄O₇: 362.23, found: 385.30 [M+Na]⁺.

### Step 2: Synthesis of tert-butyl (18E)-19-(4-nitrophenyl)-4,7,10,13,16-pentaoxanonadec-18-enoate

To a solution of tert-butyl 4,7,10,13,16-pentaoxanonadec-18-enoate (620.00 mg, 1.71 mmol, 1.00 eq) in DCM (10.00 mL) was dropwise added the solution of Grubbs 2nd (291.00 mg, 0.34 mmol, 0.20 equiv) in DCM (3.00 mL) and 1-ethenyl-4-nitrobenzene (638.00 mg, 4.28 mmol, 2.50 equiv) at room temperature. Then, the mixture was warmed to 50 degrees C. and stirred for 17 h. The resulting mixture was concentrated under reduced pressure. The residue was purified by reverse flash chromatography with the following conditions: column, C18 column; mobile phase, ACN in water (0.05% NH₄HCO₃), 5% to 70% gradient in 80 min; detector, UV 254 nm. The fractions were combined and concentrated. Tert-butyl (18E)-19-(4-nitrophenyl)-4,7,10,13,16-pentaoxanonadec-18-enoate (120.00 mg, 14.51%) was obtained as yellow oil. LC/MS: mass calcd For C₂₄H₃₇NO₉: 483.25, found: 506.20 [M+Na]⁺.

### Step 3: Synthesis of tert-butyl 19-(4-aminophenyl)-4,7,10,13,16-pentaoxanonadecanoate

The procedure was the same as tert-butyl N-[26-[(5-aminopyridin-2-yl)oxy]-3,6,9,12,15,18,21,24-octaoxahexacosan-1-yl]carbamate, but the solvent was changed to i-PrOH, the reaction time was 2.0 h. 120.00 mg of tert-butyl (18E)-19-(4-nitrophenyl)-4,7,10,13,16-pentaoxanonadec-18-enoate was used, 100.00 mg of tert-butyl 19-(4-aminophenyl)-4,7,10,13,16-pentaoxanonadecanoate was obtained as yellow oil (88.45% yield). LC/MS: mass calcd. For C₂₄H₄₁NO₇: 455.29, found: 456.25 [M+H]⁺.

### Step 4: Synthesis of tert-butyl (S)-19-(4-(2-(4-(4-chlorophenyl)-2,3,9-trimethyl-6H-thieno[3,2-f][1,2,4]triazolo[4,3-a][1,4]diazepin-6-yl)acetamido)phenyl)-4,7,10,13,16-pentaoxanonadecanoate

The procedure was the same as tert-butyl N-[23-([4-[(2S,4R)-1-acetyl-4-[(4-chlorophenyl)amino]-2-methyl-3,4-dihydro-2H-quinolin-6-yl]phenyl]formamido)-3,6,9,12,15,18,21-heptaoxatricosan-1-yl]carbamate. 80.00 mg of (S)-2-(4-(4-chlorophenyl)-2,3,9-trimethyl-6H-thieno[3,2-f][1,2,4]triazolo[4,3-a][1,4]diazepin-6-yl)acetic acid was used, 90.00 mg of tert-butyl (S)-19-(4-(2-(4-(4-chlorophenyl)-2,3,9-trimethyl-6H-thieno[3,2-f][1,2,4]triazolo[4,3-a][1,4]diazepin-6-yl)acetamido)phenyl)-4,7,10,13,16-pentaoxanonadecanoate was obtained as yellow oil (40.50% yield). LC/MS: mass calcd. For C₄₃H₅₆ClN₅O₈S: 837.35, found: 838.30 [M+H]⁺.

### Step 5: Synthesis of (S)-19-(4-(2-(4-(4-chlorophenyl)-2,3,9-trimethyl-6H-thieno[3,2-f][1,2,4]triazolo[4,3-a][1,4]diazepin-6-yl)acetamido)phenyl)-4,7,10,13,16-pentaoxanonadecanoic acid

The procedure was the same as (S)-1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatetracosan-24-oic acid. 90.00 mg of tert-butyl (S)-19-(4-(2-(4-(4-chlorophenyl)-2,3,9-trimethyl-6H-thieno[3,2-f][1,2,4]triazolo[4,3-a][1,4]diazepin-6-yl)acetamido)phenyl)-4,7,10,13,16-pentaoxanonadecanoate was used, 90.00 mg crude of (S)-19-(4-(2-(4-(4-chlorophenyl)-2,3,9-trimethyl-6H-thieno[3,2-f][1,2,4]triazolo[4,3-a][1,4]diazepin-6-yl)acetamido)phenyl)-4,7,10,13,16-pentaoxanonadecanoic acid was obtained as yellow oil. LC/MS: mass calcd. For C₃₉H₄₈ClN₅O₈S: 781.29, found: 782.50 [M+H]⁺.

### Step 6: Synthesis of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenyl)-24-methyl-19,29-dioxo-4,7,10,13,16-pentaoxa-20,24,28-triazahentriacontan-31-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as tert-butyl (R)-20-((2-(2-(2-(2-(4-(2-((S)-4-(4-chlorophenyl)-2,3,9-trimethyl-6H-thieno[3,2-f][1,2,4]triazolo[4,3-a][1,4]diazepin-6-yl)acetamido)phenoxy)ethoxy)ethoxy)ethoxy)ethyl)carbamoyl)-1-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazol-2-yl)-1,5,18-trioxo-9,12,15-trioxa-2,6,19-triazatricosan-23-oate. 80.00 mg crude of (S)-19-(4-(2-(4-(4-chlorophenyl)-2,3,9-trimethyl-6H-thieno[3,2-f][1,2,4] triazolo[4,3-a][1,4]diazepin-6-yl)acetamido)phenyl)-4,7,10,13,16-pentaoxanonadecanoic acid was used, 30.3 mg of (S)—N-(5-((3-((2-((1-(4-(2-(4-(4-chlorophenyl)-2,3,9-trimethyl-6H-thieno[3,2-f][1,2,4]triazolo[4,3-a][1,4]diazepin-6-yl)acetamido)phenyl)-24-methyl-19,29-dioxo-4,7,10,13,16-pentaoxa-20,24,28-triazahentriacontan-31-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (16.38% yield). HRMS: mass calcd. For C₈₂H₁₀₆ClN₂₃O₁₅S: 1719.7648, found: 1720.7657 [M+H]⁺.

### (S)—N-(5-((3-((2-((31-(4-(2-(4-(4-chlorophenyl)-2,3,9-trimethyl-6H-thieno[3,2-f][1,2,4]triazolo[4,3-a][1,4]diazepin-6-yl)acetamido)phenyl)-3-oxo-7,10,13,16,19,22,25,28-octaoxa-4-azahentriacontyl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 63)

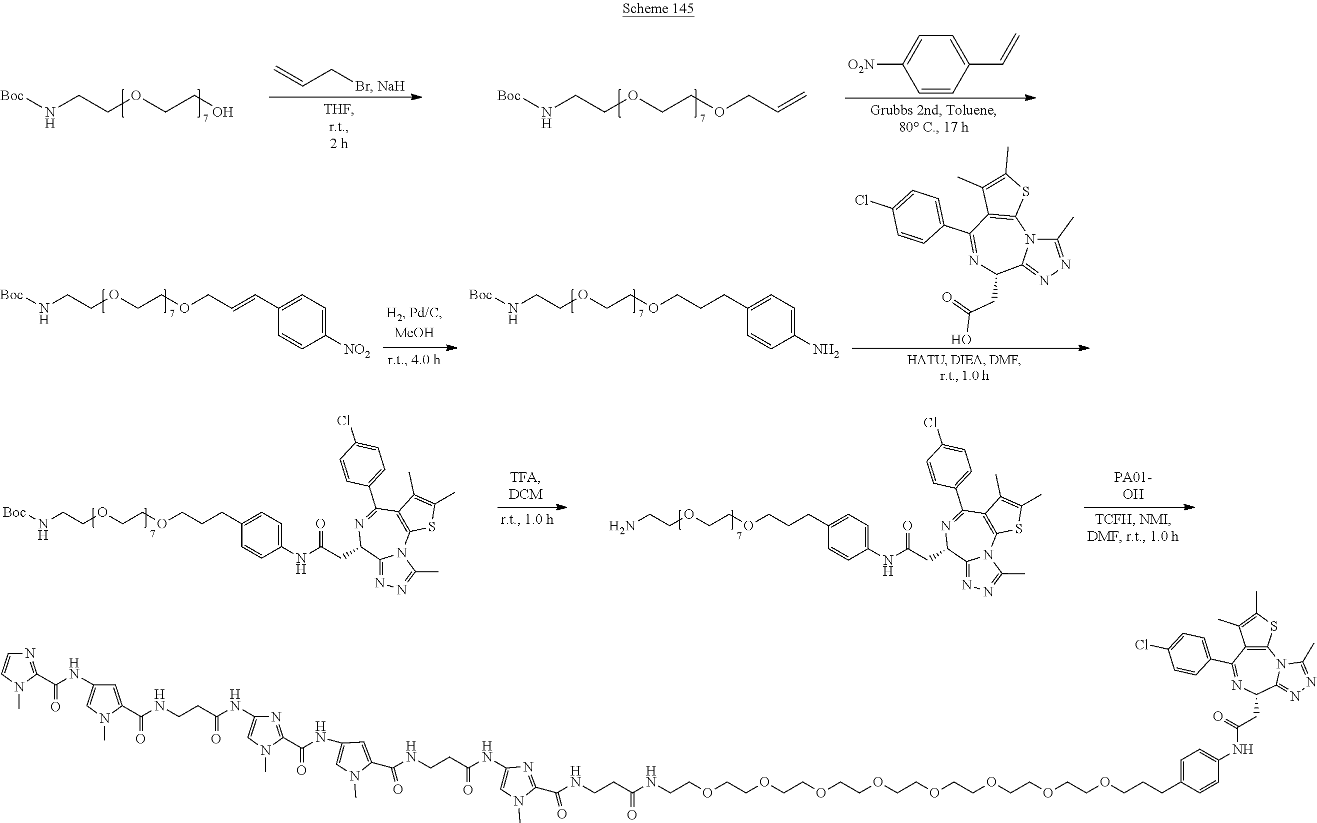

### Step 1: Synthesis of tert-butyl N-(3,6,9,12,15,18,21,24-octaoxaheptacos-26-en-1-yl)carbamate

The procedure was the same as tert-butyl 4,7,10,13,16-pentaoxanonadec-18-enoate. 300.00 mg of tert-butyl N-(23-hydroxy-3,6,9,12,15,18,21-heptaoxatricosan-1-yl)carbamate was used, 327.90 mg crude of tert-butyl N-(3,6,9,12,15,18,21,24-octaoxaheptacos-26-en-1-yl)carbamate was obtained as yellow oil. LC/MS: mass calcd. For C₂₄H₄₇NO₁₀: 509.32, found: 532.30 [M+Na]⁺.

### Step 2: Synthesis of tert-butyl N-[(26E)-27-(4-nitrophenyl)-3,6,9,12,15,18,21,24-octaoxaheptacos-26-en-1-yl]carbamate

The procedure was the same as tert-butyl (18E)-19-(4-nitrophenyl)-4,7,10,13,16-pentaoxanonadec-18-enoate, but the solvent was change to toluene, the reaction temperature was 80 degree C. and the reaction time was 17 h. 277.00 mg of tert-butyl N-(3,6,9,12,15,18,21,24-octaoxaheptacos-26-en-1-yl)carbamate was used, 141.40 mg of tert-butyl N-[(26E)-27-(4-nitrophenyl)-3,6,9,12,15,18,21,24-octaoxaheptacos-26-en-1-yl]carbamate was obtained as yellow oil (41.25% yield). LC/MS: mass calcd. For C₃₀H₅₀N₂O₁₂: 630.34, found: 653.35 [M+Na]⁺.

### Step 3: Synthesis of tert-butyl N-[27-(4-aminophenyl)-3,6,9,12,15,18,21,24-octaoxaheptacosan-1-yl]carbamate

The procedure was the same as tert-butyl N-[26-[(5-aminopyridin-2-yl)oxy]-3,6,9,12,15,18,21,24-octaoxahexacosan-1-yl]carbamate, but the solvent was changed to MeOH, the reaction time was 4.0 h. 100.00 mg of tert-butyl N-[(26E)-27-(4-nitrophenyl)-3,6,9,12,15,18,21,24-octaoxaheptacos-26-en-1-yl]carbamate was used, 51.20 mg of tert-butyl N-[27-(4-aminophenyl)-3,6,9,12,15,18,21,24-octaoxaheptacosan-1-yl] carbamate was obtained as yellow oil (53.58% yield). LC/MS: mass calcd. For C₃₀H₅₄N₂O₁₀: 602.38, found: 603.40 [M+H]⁺.

### Step 4: Synthesis of tert-butyl (S)-(27-(4-(2-(4-(4-chlorophenyl)-2,3,9-trimethyl-6H-thieno[3,2-f][1,2,4]triazolo[4,3-a][1,4]diazepin-6-yl)acetamido)phenyl)-3,6,9,12,15,18,21,24-octaoxaheptacosyl)carbamate

The procedure was the same as tert-butyl N-[26-([4-[(2S,4R)-1-acetyl-4-[(4-chlorophenyl)amino]-2-methyl-3,4-dihydro-2H-quinolin-6-yl]phenyl]formamido)-3,6,9,12,15,18,21,24-octaoxahexacosan-1-yl]carbamate. 50.00 mg of (S)-2-(4-(4-chlorophenyl)-2,3,9-trimethyl-6H-thieno[3,2-f][1,2,4]triazolo[4,3-a][1,4]diazepin-6-yl)acetic acid was used, 80.00 mg of tert-butyl (S)-(27-(4-(2-(4-(4-chlorophenyl)-2,3,9-trimethyl-6H-thieno[3,2-f][1,2,4]triazolo[4,3-a][1,4]diazepin-6-yl)acetamido)phenyl)-3,6,9,12,15,18,21,24-octaoxaheptacosyl)carbamate was obtained as yellow oil (63.45% yield). LC/MS: mass calcd. For C₄₉H₆₉ClN₆O₁₁S: 984.44, found: 1007.70 [M+Na]⁺.

### Step 5: Synthesis of (S)—N-(4-(1-amino-3,6,9,12,15,18,21,24-octaoxaheptacosan-27-yl)phenyl)-2-(4-(4-chlorophenyl)-2,3,9-trimethyl-6H-thieno[3,2-f][1,2,4]triazolo[4,3-a][1,4]diazepin-6-yl)acetamide

The procedure was the same as (S)—N-(4-((26-amino-3,6,9,12,15,18,21,24-octaoxahexacosyl)oxy)phenyl)-2-(4-(4-chlorophenyl)-2,3,9-trimethyl-6H-thieno[3,2-f][1,2,4]triazolo[4,3-a][1,4]diazepin-6-yl)acetamide. 80.00 mg of tert-butyl (S)-(27-(4-(2-(4-(4-chlorophenyl)-2,3,9-trimethyl-6H-thieno[3,2-f][1,2,4]triazolo[4,3-a][1,4]diazepin-6-yl)acetamido)phenyl)-3,6,9,12,15,18,21,24-octaoxaheptacosyl)carbamate was used, 80.00 mg crude of (S)—N-(4-(1-amino-3,6,9,12,15,18,21,24-octaoxaheptacosan-27-yl)phenyl)-2-(4-(4-chlorophenyl)-2,3,9-trimethyl-6H-thieno[3,2-f][1,2,4]triazolo[4,3-a][1,4]diazepin-6-yl)acetamide was obtained as yellow oil. LC/MS: mass calcd. For C₄₄H₆₁ClN₆O₉S: 884.39, found: 885.60 [M+H]⁺.

### Step 6; Synthesis of (S)—N-(5-((3-((2-((31-(4-(2-(4-(4-chlorophenyl)-2,3,9-trimethyl-6H-thieno[3,2-f][1,2,4]triazolo[4,3-a][1,4]diazepin-6-yl)acetamido)phenyl)-3-oxo-7,10,13,16,19,22,25,28-octaoxa-4-azahentriacontyl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide

The procedure was the same as (S)—N-(5-((3-((2-((1-(4-(2-(4-(4-chlorophenyl)-2,3,9-trimethyl-6H-thieno[3,2-f][1,2,4] triazolo[4,3-a][1,4]diazepin-6-yl)acetamido)phenoxy)-19-oxo-3,6,9,12,15-pentaoxa-18-azahenicosan-21-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide. 60.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido] imidazol-2-yl]formamido)propanoic acid was used, 20.40 mg of (S)—N-(5-((3-((2-((31-(4-(2-(4-(4-chlorophenyl)-2,3,9-trimethyl-6H-thieno[3,2-f][1,2,4]triazolo[4,3-a][1,4]diazepin-6-yl)acetamido)phenyl)-3-oxo-7,10,13,16,19,22,25,28-octaoxa-4-azahentriacontyl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide was obtained as white solid (15.81% yield). HRMS: mass calcd. For C₈₀H₁₀₂ClN₂₁O₁₇S: 1695.7172, found: 1696.7299 [M+H]⁺.

Compounds were purified by HRMS methods A or B.

Method A: Instrument: Waters Acquity I Class UPLC with Xevo G2-XSQ T of HRMS; Column: ACQUITY UPLC BEH-C18, 2.1×50 mm, 2.7 m; mobile phase A: H₂O (0.1% HCOOH), mobile B, ACN (0.1% HCOOH); Flow rate: 0.4 mL/min; Gradient: 10% B to 95% B in 1.5 min, hold 95% for another 0.5 min, then down to 10% B in 0.3 min, hold 10% B for another 0.7 min; detector: 254 nm.

Method B: Instrument: Waters Acquity I Class UPLC with Xevo G2-XS Q T of HRMS; Column: ACQUITY UPLC BEH-C18, 2.1×50 mm, 2.7 μm; mobile phase A: H₂O (0.1% HCOOH), mobile B, ACN (0.1% HCOOH); Flow rate: 0.4 mL/min; Gradient: 55% B to 40% B in 2.0 min, to 95% in another 1.5 min, hold 95% for 1.5 min, then down to 5% B in 0.3 min, hold 5% B for another 0.7 min; detector: 254 nm.

Experimental data for compounds of the disclosure purified by Method A are provided in Table 7. The found mass is shown as M+H.

TABLE 7
Mass spectrometry characterization.

| | Observed [M + H]⁺ from |
|---|---|
| Cmpd. | TOF-HRMS [m/z] |
| 1 | 1674.7498 |
| 3 | 1722.7501 |
| 4 | 1694.7559 |
| 6 | 1718.7736 |
| 7 | 1688.7509 |
| 8 | 1668.8252 |
| 9 | 1659.8629 |
| 10 | 1680.8708 |
| 11 | 1718.7765 |
| 12 | 1587.7682 |
| 13 | 1698.7059 |
| 14 | 1661.7574 |
| 16 | 1773.7657 |
| 17 | 1711.6923 |
| 18 | 1711.8661 |
| 19 | 1673.8425 |
| 20 | 1694.8518 |
| 21 | 1566.6221 |
| 22 | 1610.6553 |
| 23 | 1654.6807 |
| 24 | 1742.7294 |
| 25 | 1694.7252 |
| 26 | 1694.7240 |
| 27 | 1722.7570 |
| 28 | 1646.7905 |
| 29 | 1662.8562 |
| 30 | 1688.8383 |
| 31 | 1664.7825 |
| 32 | 1776.7324 |
| 33 | 1820.7548 |
| 34 | 1864.7745 |
| 35 | 1596.7179 |
| 36 | 1640.7479 |
| 38 | 1790.7422 |
| 39 | 1736.7289 |
| 40 | 1722.7517 |
| 41 | 1600.7404 |
| 42 | 1644.7672 |
| 43 | 1626.7821 |
| 44 | 1670.8145 |
| 45 | 1594.7822 |
| 46 | 1638.8066 |
| 47 | 1620.7660 |
| 48 | 1664.7964 |
| 49 | 1725.7118 |
| 50 | 1769.7437 |
| 51 | 1725.7189 |
| 52 | 1769.7389 |
| 53 | 1736.7681 |
| 54 | 1750.7806 |
| 55 | 1817.7858 |
| 56 | 1861.8131 |
| 57 | 1847.7949 |
| 58 | 1709.7173 |
| 59 | 1707.7401 |
| 60 | 1720.7657 |
| 62 | 1722.7520 |
| 63 | 1696.7299 |
| 65 | 1698.7102 |
| 66 | 1975.9928 |
| 67 | 1951.9484 |
| 68 | 1810.7229 |
| 69 | 1895.7407 |
| 70 | 1461.6675 |
| 71 | 1503.6971 |
| 72 | 1692.8020 |
| 73 | 1816.9139 |
| 74 | 2179.0806 |
| 75 | 1773.8661 |
| 77 | 1339.6271 |
| 78 | 1381.6415 |
| 79 | 1570.7457 |
| 80 | 1694.8671 |
| 81 | 2057.0349 |
| 82 | 1651.8245 |
| 85 | 1655.7561 |
| 86 | 1697.7723 |
| 87 | 1886.8723 |
| 88 | 2010.9990 |
| 89 | 2373.1733 |
| 90 | 1967.9456 |
| 92 | 1390.6333 |
| 96 | 2108.0398 |
| 99 | 1949.8616 |
| 100 | 1945.8893 |
| 101 | 1781.7877 |
| 102 | 1699.7012 |
| 103 | 1699.7018 |

### Pharmaceutical Compositions

### Example A-1: Parenteral Pharmaceutical Compositions

To prepare a parenteral pharmaceutical composition suitable for administration by injection (subcutaneous, intravenous), 1-1000 mg of a water-soluble salt of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s) and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 1-1000 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 1-1000 mg of a compound described herein, or a pharmaceutically acceptable salt thereof, is placed into Size 4 capsule, or size 1 capsule (hypromellose or hard gelatin) and the capsule is closed.

### Example 2. Biological Activity Assays

Expression of a target gene containing CAG or CTG repeats will be assayed by techniques known in the field. These assays include, but are not limited to quantitative reverse transcription polymerase chain reaction (RT-PCR), microarray, or multiplexed RNA sequencing (RNA-seq), with the chosen assay measuring either total expression, or the allele specific expression of the target gene. Exemplary assays are found at: Freeman W M et al., “Quantitative RT-PCR: pitfalls and potential”, BioTechniques 1999, 26, 112-125; Dudley A M et al, “Measuring absolute expression with microarrays with a calibrated reference sample and an extended signal intensity range”, PNAS USA 2002, 99(11), 7554-7559; Wang Z et al., “RNA-Seq: a revolutionary tool for transcriptomics” Nature Rev. Genetics 2009, 10, 57-63.

Production of the translation product of the target gene will be assayed by techniques known in the field. These assays include, but are not limited to Western blot assay, with the chosen assay measuring either total protein expression, or allele specific expression of the target gene.

For use in assay, two tissue models and two animal models are contemplated.

### Example B1: EC₅₀ Assay

Cell culture: Cells were cultured in RPMI1640 medium+15% FBS. Cells were maintained at a density between 2e5/mL and 1e6/mL. Cells were centrifuged, resuspended in fresh medium, counted and plated at 150,000 cells per well in 100 uL in a non-coated, flat bottom tissue culture plate.

Compound treatment: 10 mM stock solution of FA GeneTAC was diluted 1:10 in DMSO followed by a 1:100 dilution in growth medium. Working solution was then further diluted to 10× desired final concentration of 150 nM. Compound was then diluted at a 1:3 ratio into growth medium containing 0.01% DMSO. 5-point, 3-fold dose response curve was generated. 11 μL of 10× compound was added to wells containing 100 μL cell suspension of GM15850. 11 μL growth medium containing 0.01% DMSO was added to all wells not treated with FA GeneTAC. Cells were allowed to incubate for 48 hrs prior to cell lysis using guanidine isothiocyanate solution.

RNA isolation: Total RNA was isolated and purified in 384-well column filter plates using chaotropic salt.

qRT-PCR: qRT-PCR reactions were assembled using AgPath-ID reagents (Thermo Fisher) using 6 uL mastermix and 4 μL RNA. qRT-PCR TaqMan primer probe sets against human FXN (Assay ID Hs01075496_m1) and human GAPDH (Assay ID Hs00266705_g1) were used to measure the intended targets. qRT-PCR was run on the ThermoFisher QuantStudio 6 PRO instrument using the manufacturer's recommended cycling conditions.

Data analysis: qPCR data was analyzed using Thermo Fisher Design and Analysis software.

Data was exported to Excel and hFXN expression was normalized to hGAPDH expression.

Representative in vitro biochemical data is presented in Table 8. A<100 nM; B is 100 nM to 500 nM; C >500 nM.

TABLE 8
In vitro potency data.

| | EC₅₀ WT |
|---|---|
| Cmpd. No. | (nM, 48 hr) |
| 3 | B |
| 6 | B |
| 8 | A |
| 9 | B |
| 10 | B |
| 11 | C |
| 13 | A |
| 16 | B |
| 17 | A |
| 19 | A |
| 20 | B |
| 21 | B |
| 22 | B |
| 23 | B |
| 24 | A |
| 25 | B |
| 26 | B |
| 29 | B |
| 30 | C |
| 31 | A |
| 33 | B |
| 35 | A |
| 36 | A |
| 38 | A |
| 39 | A |
| 40 | A |
| 41 | B |
| 42 | A |
| 43 | C |
| 44 | B |
| 45 | B |
| 46 | B |
| 48 | C |
| 49 | A |
| 50 | A |
| 51 | A |
| 52 | A |
| 53 | B |
| 54 | B |
| 55 | B |
| 56 | B |
| 57 | B |
| 58 | A |
| 59 | A |
| 60 | B |
| 62 | A |
| 63 | A |
| 65 | B |
| 71 | C |
| 73 | C |
| 75 | C |
| 86 | C |
| 87 | B |
| 88 | C |
| 89 | C |
| 90 | B |
| 99 | A |
| 100 | B |
| 101 | B |
| 102 | A |
| 103 | A |

### Example 3. Clinical Trials in Freidriech's Ataxia

Brief Summary: The primary objective of the study is to evaluate the efficacy (using the modified Friedreich Ataxia Rating Scale [mFARS]) and safety of the compounds disclosed herein in participants with Friedreich ataxia.

Detailed Description: During the double-blind, placebo-controlled phase, participants will be stratified by baseline mFARS score (<40 versus ≥40), age of disease onset (<14 versus ≥14), and age at screening (≤21 years or >21 years) and randomized to receive either a compound disclosed herein or placebo using interactive web response system (IWRS). Following completion of the randomized, double-blind, placebo-controlled phase (72 weeks), participants will enter into an open-label extension phase (24 weeks) during which they will receive open-label treatment with a compound disclosed herein at the dose they received in the randomized phase of the study (for participants entering the extension phase who initially received placebo, the dose of the compound disclosed herein will be determined based on age and weight) and then a safety follow-up (10-30 days after last dose.

Primary outcomes: Change From Baseline in the Modified Friedreich Ataxia Rating Scale (mFARS) Score at Week 72 [Time Frame: Baseline, Week 72]

Friedreich Ataxia Rating Scale (FARS) is a disease-specific scale that measures progression of neurological effects of FA. The mFARS is a validated and reliable 93-item scale; comprised of the neurologic component of the FARS and evaluates bulbar, upper limb, lower limb, and upright stability/gait function. For each item, responses categorize the corresponding neurological finding, and the findings are assigned a score ranging from 0 to 3, 4, or 5 with 0 being normal and higher numbers indicative of greater impairment.

Secondary Outcomes:

(1) Change from Baseline in Friedreich Ataxia Rating Scale Activities of Daily Living (FARS-ADL) Score at Week 72 [Time Frame: Baseline, Week 72]

The FARS-ADL is a subsection of the FARS questionnaire that assesses activities of daily living, including speech, personal hygiene, feeding, and mobility. Participants rank each category using a scale of 0 (normal) to 4 (severe disability/inability to carry out activity independently), with lower scores indicative of “normal” function/activity.

(2) Change from Baseline in 1-Minute Walk Test (1MWT) at Week 72 [Time Frame: Baseline, Week 72]

The 1MWT is a timed performance test used to measure functional ability, walking endurance, balance, and muscle performance by measuring maximal walking speed in 1 minute. Participants will be instructed to walk as quickly as possible for 1 minute without running. Maximal walking speed will be measured upon completion of the walk and recorded.

(3) Number of Falls Through Week 72 [Time Frame: Baseline Through Week 72]

The fall log directly relate to a participant's ability to ambulate during normal daily activities. Thus, each participant will be required to maintain a fall log, which will include the date and time of each fall. Falls as defined by World Health Organization as “inadvertently coming to rest on the ground, floor or other lower level, excluding intentional change in position to rest in furniture, wall or other objects,” will be reported.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A transcription modulator molecule having a first terminus, a second terminus, and an oligomeric backbone, wherein:
a) the first terminus comprises a DNA-binding moiety capable of noncovalently binding to a nucleotide repeat sequence GAA;
b) the second terminus comprises a protein-binding moiety binding to a regulatory molecule that modulates an expression of a gene comprising the nucleotide repeat sequence GAA; and
c) the oligomeric backbone comprising a linker between the first terminus and the second terminus.

2. The transcription modulator molecule of claim 1, wherein the first terminus comprises a polyamide selected from the group consisting of a linear polyamide, a hairpin polyamide, a H-pin polyamide, an overlapped polyamide, a slipped polyamide, a cyclic polyamide, a tandem polyamide, and an extended polyamide.

3. The transcription modulator molecule of claim 1 or 2, wherein the first terminus comprises a linear polyamide.

4. The transcription modulator molecule of any one of claims 1-3, wherein the polyamide is capable of binding the DNA with an affinity of less than 500 nM.

5. The transcription modulator molecule of any one of claims 1-4, wherein the first terminus comprises —NH-Q-C(O)—, wherein Q is an optionally substituted C₆₋₁₀ arylene, optionally substituted 4-10 membered heterocyclene, optionally substituted 5-10 membered heteroarylene group, or an optionally substituted alkylene group.

6. The transcription modulator molecule of any one of claims 1-5, wherein the first terminus comprises at least three heteroaromatic carboxamide moieties comprising at least one heteroatom selected from O, N, and S, and at least one aliphatic amino acid residue chosen from the group consisting of glycine, β-alanine, γ-aminobutyric acid, 2,4-diaminobutyric acid, and 5-aminovaleric acid.

7. The transcription modulator molecule of claim 6, wherein the heteroaromatic carboxamide moiety is a monocyclic or bicyclic moiety.

8. The transcription modulator molecule of claim 6, wherein the first terminus comprises one or more carboxamide moieties selected from the group consisting of optionally substituted pyrrole carboxamide monomer, optionally substituted imidazole carboxamide monomer, and β-alanine monomer.

9. The transcription modulator molecule of any one of claims 1-8, wherein the first terminus comprises a structure of Formula (A-1), or a pharmaceutically acceptable salt thereof:
-L₁ₐ-[A-M]ₚ-E₁ Formula (A-1),
wherein:
each [A-M] appears p times and p is an integer in the range of 1 to 10;
L₁ₐ is a bond, a C₁₋₆ alkylene, —NR^{a}—C₁₋₆ alkylene-C(O)—, —NR^{a}C(O)—, —NR^{a}—C₁₋₆ alkylene, —O—, or —O—C₁₋₆ alkylene;
each A is selected from the group consisting of a bond, C₁₋₁₀ alkylene, optionally substituted C₆₋₁₀ arylene group, optionally substituted 4-10 membered heterocyclene, optionally substituted 5-10 membered heteroarylene group, —C₁₋₁₀ alkylene-C(O)—, —C₁₋₁₀ alkylene-NR^{a}—, —CO—, —NR^{a}—CONR^{a}—, —CONR^{a}C₁₋₄alkylene-, —NR^{a}CO—C₁₋₄alkylene-, —C(O)O—, —O—, —S—, —S(O)—, —S(O)₂—, —C(═S)—NH—, —C(O)—NH—NH—, —C(O)—N═N—, —C(O)—CH═CH—, (CH₂)₀₋₄—CH═CH—(CH₂)₀₋₄, —N(CH₃)—C₁₋₆ alkylene, 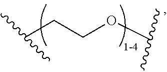
-NH—C₁₋₆ alkylene-NH—, —O—C₁₋₆ alkylene-O—, —NH—N═N—, —NH—C(O)—NH—, and any combinations thereof, and at least one A is —CONH—;
each M is an optionally substituted C₆₋₁₀ arylene group, optionally substituted 4-10 membered heterocyclene, optionally substituted 5-10 membered heteroarylene group, or an optionally substituted alkylene;
E₁ is H or -A^{E}-G;
A^{E} is absent or —NHCO—;
G is selected from the group consisting of optionally substituted H, C₆₋₁₀ aryl, optionally substituted 4-10 membered heterocyclyl, optionally substituted 5-10 membered heteroaryl, an optionally substituted C₁₋₆ alkyl, C₀₋₄ alkylene-NHC(═NH)NH, —CN, —C₀₋₄alkylene-C(═NH)(NR^{a}R^{b}), —C₀₋₄alkylene-C(═N⁺H₂)—(NR^{a}R^{b}), —C₁₋₅alkylene-NR^{a}R^{b}, C₀₋₄ alkylene-NHC(═NH)R^{a}, and optionally substituted amine; and
each R^{a} and R^{b} are independently selected from the group consisting of H, an optionally substituted C₁₋₆ alkyl, an optionally substituted C₃₋₁₀ cycloalkyl, optionally substituted C₆₋₁₀ aryl, optionally substituted 4-10 membered heterocyclyl, and optionally substituted 5-10 membered heteroaryl.

10. The transcription modulator molecule of any one of claims 1-8, wherein the first terminus comprises a structure of Formula (A-2), or a pharmaceutically acceptable salt thereof: 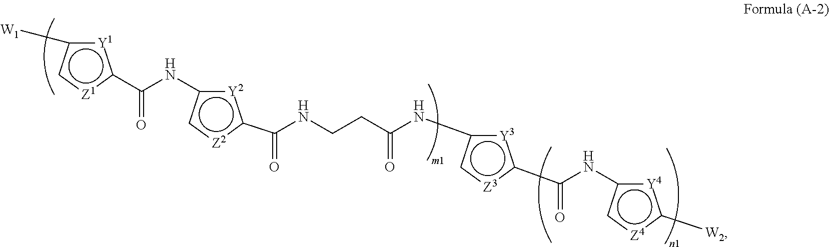
wherein:
each Y¹, Y², Y³, and Y⁴ is independently CH or N;
each Z¹, Z², Z³ and Z⁴ is independently O, S, or NR^{1D};
W₁ is hydrogen, optionally substituted C₁-C₆ alkyl, —C(O)—NR^{1E}R^{1F}, —NR^{1E}—C(O)—NR^{1E}R^{1F}, or (AA)₁₋₁₀;
W₂ is hydrogen, optionally substituted C₁-C₆ alkyl, —C(O)—NR^{1E}R^{1F}, or (AA)₁₋₁₀;
m1 is 1, 2, 3, or 4;
n1 is 0, 1, or 2; and
AA is an amino acid residue selected from β-alanine, lysine, and arginine;
each R^{1D} and R^{1E} is independently hydrogen or C₁-C₆ alkyl; and
R^{1F} is hydrogen, an optionally substituted C₁-C₁₀ alkyl, C₁-C₁₀ heteroalkyl, PEG₁₋₂₀, or one or more AA.

11. The transcription modulator molecule of claim 10, wherein the first terminus comprises a structure of Formula (A-3), or a pharmaceutically acceptable salt thereof:

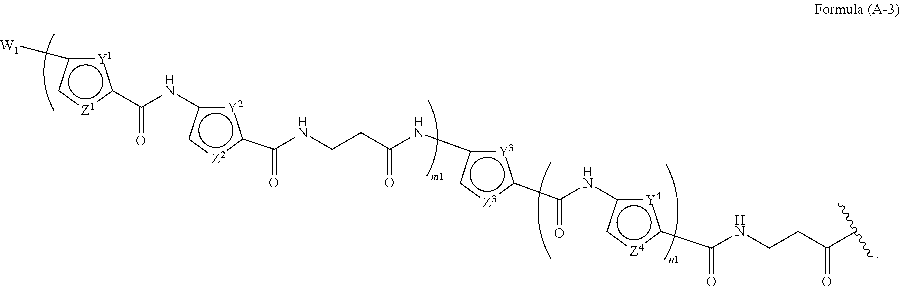

12. The transcription modulator molecule of claim 10 or 11, wherein each Z¹, Z², Z³, and Z⁴ is independently NR^{1D}, wherein R^{1D} is C₁-C₆ alkyl.

13. The transcription modulator molecule of claim 10 or 11, wherein each Z¹, Z², Z³, and Z⁴ is independently NCH₃.

14. The transcription modulator molecule of any one of claims 10-13, wherein each Z¹ and Z³ are N; and each Z² and Z⁴ are independently CH or N.

15. The transcription modulator molecule of claim 14, wherein each Z² and Z⁴ is CH.

16. The transcription modulator molecule of any one of claims 10-15, wherein the first terminus comprises a structure of Formula (A-4):

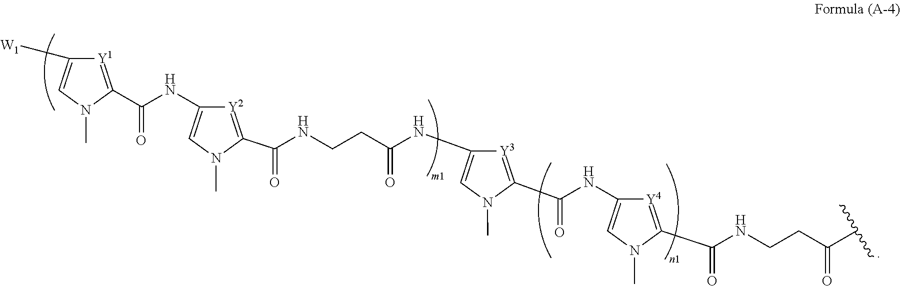

17. The transcription modulator molecule of any one of claims 10-16, wherein W₁ is optionally substituted C₁-C₆ alkyl, or —C(O)—NR^{1E}R^{1F}.

18. The transcription modulator molecule of claim 17, wherein W₁ is —C(O)—NR^{1E}R^{1F}, wherein R^{1E} is hydrogen; and R^{1F} is hydrogen, optionally substituted C₁-C₁₀ alkyl, or PEG₁₋₂₀.

19. The transcription modulator molecule of any one of claims 10-16, wherein W₁ is hydrogen.

20. The transcription modulator molecule of any one of claims 10-19, wherein m1 is 2 or 3; and n1 is 0 or 1.

21. The transcription modulator molecule of claim 20, wherein n1 is 0.

22. The transcription modulator molecule of claim 20, wherein n1 is 1.

23. The transcription modulator molecule of any one of claims 1-22, wherein the linker has a length of less than about 50 Angstroms.

24. The transcription modulator molecule of any one of claims 1-22, wherein the linker has a length of about 15 to 40 Angstroms.

25. The transcription modulator molecule of any one of claims 1-22, wherein the linker comprises between 5 and 50 chain atoms.

26. The transcription modulator molecule of any one of claims 1-22, wherein the linker comprises a multimer having from 2 to 50 spacing moieties, and wherein the spacing moiety is independently selected from the group consisting of —((CR^{3a}R^{3b})ₓ—O)_{y}—, —((CR^{3a}R^{3b})ₓ—NR^{4a})_{y}—, —((CR^{3a}R^{3b})ₓ—CH═CH—(CR^{3a}R^{3b})ₓ—O)_{y}—, optionally substituted —C₁₋₁₂ alkyl, optionally substituted C₂₋₁₀ alkenyl, optionally substituted C₂₋₁₀ alkynyl, optionally substituted C₆₋₁₀ arylene, optionally substituted C₃₋₇ cycloalkylene, optionally substituted 5- to 10-membered heteroarylene, optionally substituted 4- to 10-membered heterocycloalkylene, an amino acid residue, —O—, —C(O)NR^{4a}—, —NR^{4a}C(O)—, —C(O)—, —NR^{4a}—, —C(O)O—, —O—, —S—, —S(O)—, —SO₂—, —SO₂NR^{4a}—, —NR^{4a}SO₂—, and —P(O)OH—, and any combinations thereof; wherein
each x is independently 2-4;
each y is independently 1-10;
each R^{3a} and R^{3b} are independently selected from hydrogen, optionally substituted alkyl, optionally substituted alkenyl, optionally substituted alkynyl, optionally substituted alkoxy, optionally substituted amino, carboxyl, carboxyl ester, acyl, acyloxy, acyl amino, amino acyl, optionally substituted alkylamide, sulfonyl, optionally substituted thioalkoxy, optionally substituted aryl, optionally substituted heteroaryl, optionally substituted cycloalkyl, and optionally substituted heterocyclyl; and
each R^{4a} is independently a hydrogen or an optionally substituted C₁₋₆ alkyl.

27. The transcription modulator molecule of any one of claims 1-26, wherein the oligomeric backbone comprises -(T¹-V¹)ₐ-(T²-V²)_{b}-(T³-V³)_{c}-(T⁴-V⁴)_{d}-(T⁵-V⁵)ₑ—,
wherein a, b, c, d and e are each independently 0 or 1, and where the sum of a, b, c, d and e is 1 to 5;
T¹, T², T³, T⁴ and T⁵ are each independently selected from an optionally substituted (C₁-C₁₂) alkylene, optionally substituted alkenylene, optionally substituted alkynylene, (EA)_{w}, (EDA)ₘ, (PEG)ₙ, (modified PEG)ₙ, (AA)ₚ, —(CR^{2a}OH)ₕ—, optionally substituted (C₆-C₁₀) arylene, optionally substituted C₃₋₇ cycloalkylene, optionally substituted 5- to 10 membered heteroarylene, optionally substituted 4- to 10-membered heterocycloalkylene, a disulfide, a hydrazine, a carbohydrate, a beta-lactam, and an ester;
each m, p, and w are independently an integer from 1 to 20;
n is an integer from 1 to 30;
h is an integer from 1 to 12;
EA has the following structure: 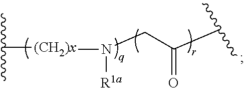
EDA has the following structure: 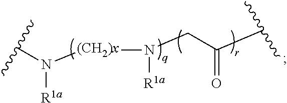
wherein each q is independently an integer from 1 to 6;
each x is independently an integer from 2 to 4 and
each r is independently 0 or 1;
(PEG)ₙ has the structure of —(CR^{2a}R^{2b}—CR^{2a}R^{2b}—O)ₙ—CR^{2a}R^{2b}—;
(modified PEG)ₙ has the structure of replacing at least one —(CR^{2a}R^{2b}—CR^{2a}R^{2b}—O)— in (PEG)ₙ with —(CH₂—CR^{2a}═CR^{2a}—CH₂—O)— or —(CR^{2a}R^{2b}—CR^{2a}R^{2b}S)—;
AA is an amino acid residue;
V¹, V², V³, V⁴ and V⁵ are each independently selected from the group consisting of a bond, —CO—, —NR^{1a}—, —CONR^{1a}—, —NR^{1a}CO—, —CONR^{1a}C₁₋₄ alkyl-, —NR^{1a}CO—C₁₋₄ alkyl-, —C(O)O—, —OC(O)—, —O—, —S—, —S(O)—, —SO₂—, —SO₂NR^{1a}—, —NR^{1a}SO₂— and —P(O)OH—;
each R^{1a} is independently hydrogen or and optionally substituted C₁₋₆ alkyl; and each R^{2a} and R^{2b} are independently selected from hydrogen, alkyl, substituted alkyl, alkenyl, substituted alkenyl, alkynyl, substituted alkynyl, halogen, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

28. The transcription modulator molecule of claim 27, wherein T¹, T², T³, T⁴, and T⁵ are each independently selected from (C₁-C₁₂)alkyl, substituted (C₁-C₁₂)alkyl, (EA)_{w}, (EDA)ₘ, (PEG)ₙ, (modified PEG)ₙ, (AA)ₚ, —(CR^{2a}OH)ₕ—, an optionally substituted phenyl, piperidin-4-amino (P4A), piperidine-3-amino, piperazine, pyrrolidin-3-amino, azetidine-3-amino, para-amino-benzyloxycarbonyl (PABC), meta-amino-benzyloxycarbonyl (MABC), para-amino-benzyloxy (PABO), meta-amino-benzyloxy (MABO), para-aminobenzyl, an acetal group, a disulfide, a hydrazine, a carbohydrate, a beta-lactam, an ester, (AA)ₚ-MABC-(AA)ₚ, (AA)ₚ-MABO-(AA)ₚ, (AA)ₚ-PABO-(AA)ₚ and (AA)ₚ-PABC-(AA)ₚ.

29. The transcription modulator molecule of claim 27, wherein T¹, T², T³, T⁴ and T⁵ are each independently selected from (C₁-C₁₂)alkyl, substituted (C₁-C₁₂)alkyl, (EA)_{w}, (EDA)ₘ, (PEG)ₙ, (modified PEG)ₙ, (AA)ₚ, —(CR^{2a}OH)ₕ—, optionally substituted (C₆-C₁₀) arylene, 4-10 membered heterocycloalkene, and optionally substituted 5-10 membered heteroarylene.

30. The transcription modulator molecule of claim 27, wherein T⁴ or T⁵ is an optionally substituted (C₆-C₁₀) arylene.

31. The transcription modulator molecule of claim 27, wherein T⁴ or T⁵ is an optionally substituted phenylene.

32. The transcription modulator molecule of any one of claims 1-28, wherein the linker comprises —N(R^{1a})(CH₂)ₓN(R^{1b})(CH₂)ₓN—, wherein R^{1a} and R^{1b} are each independently selected from hydrogen or optionally substituted C₁-C₆ alkyl; and each x is independently an integer in the range of 1-6.

33. The transcription modulator molecule of any one of claims 1-32, wherein the linker comprises —(CH₂—C(O)N(R″)—(CH₂)_{q}—N(R′)—(CH₂)_{q}—N(R″)C(O)—(CH₂)ₓ—C(O)N(R″)-A-, —(CH₂)ₓ—C(O)N(R″)—(CH₂CH₂O)_{y}(CH₂)ₓ—C(O)N(R″)-A-, —C(O)N(R″)—(CH₂)_{q}—N(R′)—(CH₂)_{q}—N(R″)C(O)—(CH₂)ₓ-A-, —(CH₂)ₓ—O—(CH₂CH₂O)_{y}—(CH₂)ₓ—N(R″)C(O)—(CH₂)ₓ-A-, or —N(R″)C(O)—(CH₂)—C(O)N(R″)—(CH₂)ₓ—O(CH₂CH₂O)_{y}(CH₂)ₓ-A-; wherein R′ is methyl; R″ is hydrogen; each x and y are independently an integer from 1 to 10; each q is independently an integer from 2 to 10; and each A is independently selected from a bond, an optionally substituted C₁₋₁₂ alkyl, an optionally substituted C₆₋₁₀ arylene, optionally substituted C₃₋₇ cycloalkylene, optionally substituted 5- to 10-membered heteroarylene, and optionally substituted 4- to 10-membered heterocycloalkylene.

34. The transcription modulator molecule of any one of claims 1-33, wherein the linker comprises PEG (polyethylene glycol).

35. The transcription modulator molecule of claim 34, wherein the linker comprises 2-20 PEG units.

36. The transcription modulator molecule of claim 34, wherein the linker comprises 6, 8, 10, or 15 PEG units.

37. The transcription modulator molecule of any one of claims 1-36, wherein the linker is joined with the first terminus with a group selected from —CO—, —NR^{1a}—, —CONR^{1a}—, —NR^{1a}CO—, —CONR^{1a}C₁₋₄alkyl-, —NR^{1a}CO—C₁₋₄alkyl-, —C(O)O—, —OC(O)—, —O—, —S—, —S(O)—, —SO₂—, —SO₂NR^{1a}—, —NR^{1a}SO₂—, —P(O)OH—, —((CH₂)ₓ—O)—, —((CH₂)_{y}—NR^{1a})—, optionally substituted —C₁₋₁₂ alkylene, optionally substituted C₂₋₁₀ alkenylene, optionally substituted C₂₋₁₀ alkynylene, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene; wherein each x and y are independently 1-4, and each R^{1a} is independently a hydrogen or optionally substituted C₁₋₆ alkyl.

38. The transcription modulator molecule of any one of claims 1-36, wherein the linker is joined with the first terminus with a group selected from —CO—, —NR^{1a}—, C₁₋₁₂ alkyl, —CONR^{1a}—, and —NR^{1a}CO—; wherein each R^{1a} is independently a hydrogen or optionally substituted C₁₋₆ alkyl.

39. The transcription modulator molecule of any one of claims 1-35, wherein the second terminus comprises one or more optionally substituted C₆₋₁₀ aryl, optionally substituted C₄₋₁₀ carbocyclic, optionally substituted 4 to 10 membered heterocyclic, or optionally substituted 5 to 10 membered heteroaryl.

40. The transcription modulator molecule of any one of claims 1-39, wherein the second terminus comprises at least one 5-10 membered heteroaryl group having at least two nitrogen atoms.

41. The transcription modulator molecule of any one of claims 1-40, wherein the second terminus comprises a moiety capable of binding to the regulatory protein, and the moiety is from a compound capable of binding to the regulatory protein.

42. The transcription modulator molecule of any one of claims 1-40, wherein the second terminus comprises at least one group selected from an optionally substituted diazine, an optionally substituted diazepine, and an optionally substituted phenyl.

43. The transcription modulator molecule of any one of claims 1-40, wherein the second terminus comprises a moiety that binds to a bromodomain protein.

44. The transcription modulator molecule of any one of claims 1-43, wherein the second terminus comprises a diazine or diazepine ring, wherein the diazine or diazepine ring is fused with a C₆₋₁₀ aryl or a 5-10 membered heteroaryl ring comprising one or more heteroatom selected from S, N and O.

45. The transcription modulator molecule of any one of claims 1-43, wherein the second terminus comprises an optionally substituted bicyclic or tricyclic structure.

46. The transcription modulator molecule of claim 45, wherein the optionally substituted bicyclic or tricyclic structure comprises a diazepine ring fused with a thiophene ring.

47. The transcription modulator molecule of claim 46, wherein the second terminus comprises an optionally substituted bicyclic structure, wherein the bicyclic structure comprises a diazepine ring fused with a thiophene ring.

48. The transcription modulator molecule of claim 45, wherein the second terminus comprises an optionally substituted tricyclic structure, wherein the tricyclic structure is a diazepine ring that is fused with a thiophene and a triazole.

49. The transcription modulator molecule of any one of claims 1-43, wherein the second terminus comprises a compound having the structure of Formula 7: 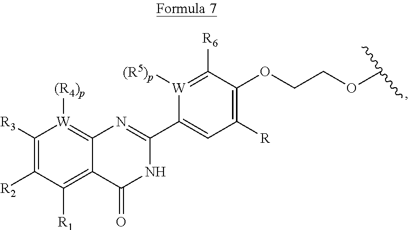
wherein;
R₁ and R₃ are each independently selected from alkoxy, alkyl, amino, halogen, and hydrogen;
R₂ is selected from alkoxy, alkyl, alkenyl, alkynyl, amide, amino, halogen, and hydrogen;
R₆ and R₇ are each independently selected from alkyl, alkoxy, amino, halogen, and hydrogen;
R₅ is hydrogen;
each W is independently selected from C and N, wherein if W is N, then p is 0 or 1, and if W is C, then p is 1; for W—(R₄)ₚ, if W is C, p is 1 and R₄ is H, or if W is N, then p is 0; if R₁ is hydrogen, then R₃ is alkoxy; if R₃ is hydrogen, then R₁ is selected from amino and alkoxy; and at least one of R₆ and R₇ is independently selected from alkyl, alkoxy, amino, and halogen.

50. The transcription modulator molecule of any one of claims 1-43, wherein the second terminus comprises a compound of Formula 8:

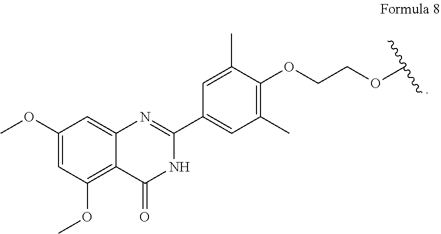 [SMILES: *OCCOc1c(C)cc(-c2nc3cc(OC)cc(OC)c3c(=O)[nH]2)cc1C]

51. The transcription modulator molecule of any one of claims 1-40, wherein the second terminus comprises a compound having the structure of Formula (9-A), or a pharmaceutically acceptable salt thereof: 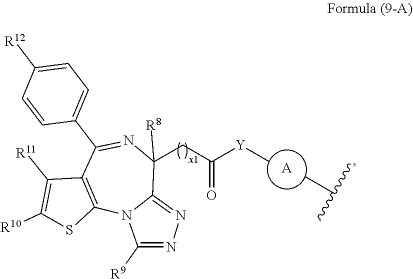
wherein;
Ring A is absent or 6-membered monocyclic aryl or heteroaryl;
Y is —NH— or —O—;
R⁹, R¹⁰, and R¹¹ are each independently selected from hydrogen, optionally substituted C₁₋₆ alkyl, C₁₋₆ haloalkyl, or C₁₋₆ hydroxyalkyl;
R¹² is selected from hydrogen, halogen, —NO₂, —CN, optionally substituted aryl, optionally substituted C₁₋₆ alkyl, C₁₋₆ haloalkyl, or C₁₋₆ hydroxyalkyl;
or R¹² is —NR^{A}R^{B}, wherein
R^{A} and R^{B} are each independently hydrogen, optionally substituted C₁₋₆ alkyl or C₁₋₆ heteroalkyl; and
x1 is an integer from 1-6.

52. The transcription modulator molecule of claim 51, wherein the second terminus comprises a compound having the structure of Formula (9-C), or a pharmaceutically acceptable salt thereof:

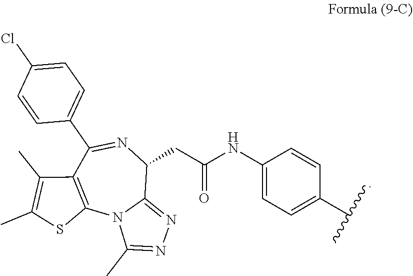 [SMILES: *c1ccc(NC(=O)C[C@H]2N=C(c3ccc(Cl)cc3)c3c(sc(C)c3C)-n3c(C)nnc32)cc1]

53. The transcription modulator molecule of any one of claims 1-43, wherein the second terminus comprises a compound having the structure of Formula (10-A), or a pharmaceutically acceptable salt thereof: 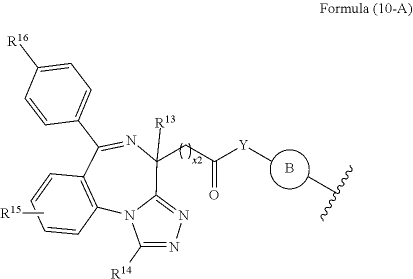
wherein;
Ring B is absent or 5-6-membered monocyclic aryl or heteroaryl or 4-8-membered heterocycle;
Y is —NH— or —O—;
R¹³ is selected from hydrogen or optionally substituted C₁-C₆ alkyl;
R¹⁴ and R¹⁵ are each independently selected from hydrogen, optionally substituted C₁₋₆ alkyl, C₁₋₆ haloalkyl, or C₁₋₆ hydroxyalkyl;
R¹⁶ is selected from hydrogen, halogen, —NO₂, —CN, optionally substituted aryl, optionally substituted C₁₋₆ alkyl, C₁₋₆ haloalkyl, or C₁₋₆ hydroxyalkyl;
or R¹⁶ is —NR^{A}R^{B}, wherein
R^{A} and R^{B} are each independently hydrogen, optionally substituted C₁₋₆ alkyl or C₁₋₆ heteroalkyl; and
x2 is an integer from 1-6.

54. The transcription modulator molecule of claim 53, wherein the second terminus comprises a compound having the structure of Formula (10-B), or a pharmaceutically acceptable salt thereof:

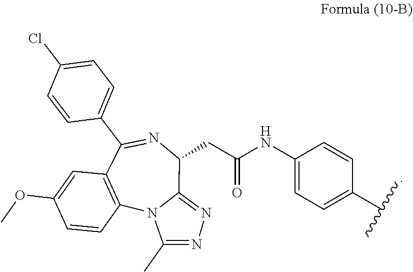 [SMILES: *c1ccc(NC(=O)C[C@H]2N=C(c3ccc(Cl)cc3)c3cc(OC)ccc3-n3c(C)nnc32)cc1]

55. The transcription modulator molecule of any one of claims 1-43, wherein the second terminus comprises a compound having the structure of Formula (12-A): 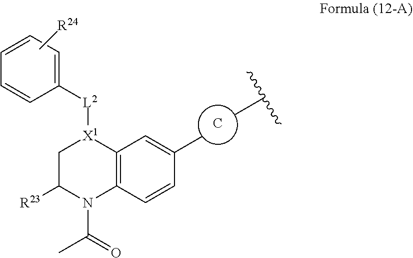
wherein;
Ring C is absent or monocyclic 6-membered aryl or heteroaryl;
X¹ is CH or N;
L² is —NR^{D}— or —CR^{D}H—
R²³ is C₁-C₆ alkyl or C₃-C₆ cycloalkyl; and
R²⁴ is halogen, alkyl, hydroxyalkyl, haloalkyl; optionally substituted C₁-C₆ alkyl, C₁-C₆ haloalkyl or C₁-C₆ hydroxyalkyl; and
R^{D} is hydrogen or C₁₋₃ alkyl.

56. The transcription modulator molecule of claim 55, wherein Ring C is:

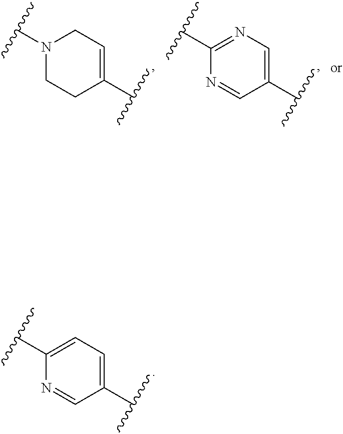 [SMILES: *C1=CCN(*)CC1 ; *c1cnc(*)nc1 ; *c1ccc(*)nc1]

57. The transcription modulator molecule of claim 55, wherein Ring C is phenyl.

58. The transcription modulator molecule of claim 55 or 57, wherein the second terminus comprises a compound having the structure of Formula (12-B), or a pharmaceutically acceptable salt thereof:

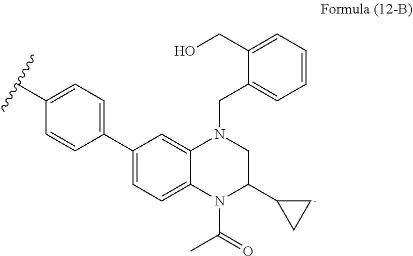 [SMILES: *c1ccc(-c2ccc3c(c2)N(Cc2ccccc2CO)CC(C2CC2)N3C(C)=O)cc1]

59. The transcription modulator molecule of claim 52 or 54, wherein the second terminus comprises a compound having the structure of Formula (12-C), or a pharmaceutically acceptable salt thereof:

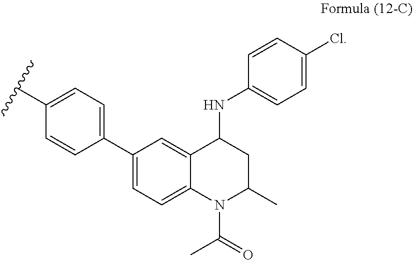 [SMILES: *c1ccc(-c2ccc3c(c2)C(Nc2ccc(Cl)cc2)CC(C)N3C(C)=O)cc1]

60. The transcription modulator molecule of any one of claims 1-43 wherein the second terminus is selected from: 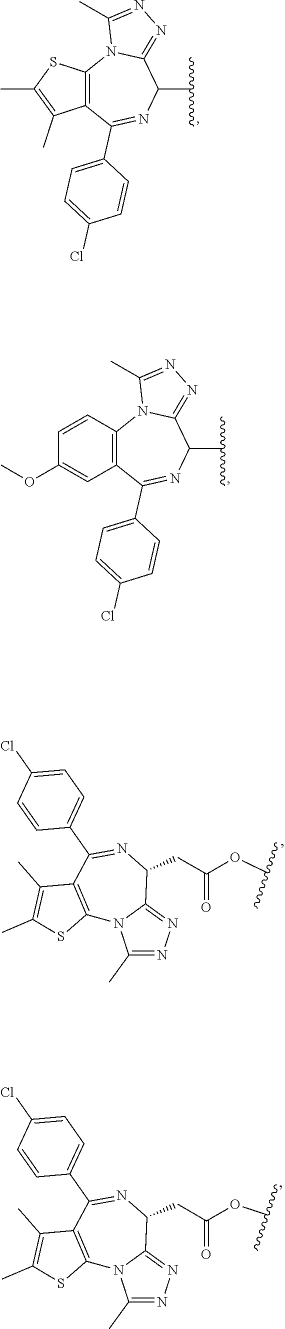 [SMILES: *C1N=C(c2ccc(Cl)cc2)c2c(sc(C)c2C)-n2c(C)nnc21 ; *C1N=C(c2ccc(Cl)cc2)c2cc(OC)ccc2-n2c(C)nnc21 ; *OC(=O)C[C@H]1N=C(c2ccc(Cl)cc2)c2c(sc(C)c2C)-n2c(C)nnc21 ; *OC(=O)C[C@H]1N=C(c2ccc(Cl)cc2)c2c(sc(C)c2C)-n2c(C)nnc21] 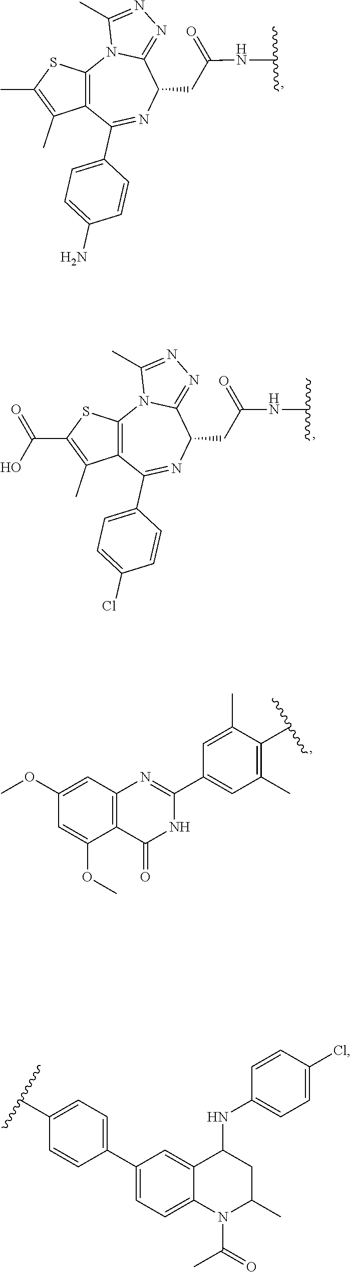 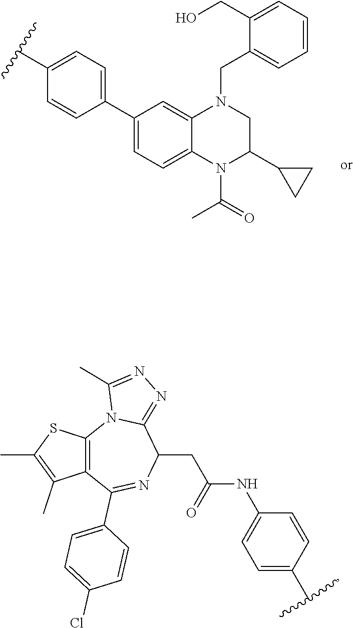 [SMILES: *c1ccc(-c2ccc3c(c2)N(Cc2ccccc2CO)CC(C2CC2)N3C(C)=O)cc1 ; *c1ccc(NC(=O)CC2N=C(c3ccc(Cl)cc3)c3c(sc(C)c3C)-n3c(C)nnc32)cc1]
or a pharmaceutically acceptable salt thereof.

61. The transcription modulator molecule of any one of claims 1-43, wherein the protein binding moiety is: 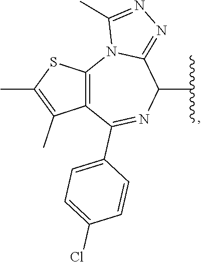 [SMILES: *C1N=C(c2ccc(Cl)cc2)c2c(sc(C)c2C)-n2c(C)nnc21]
or a pharmaceutically acceptable salt thereof.

62. A transcription modulator molecule as recited in any one of the proceeding claims for use as a medicament.

63. A transcription modulator molecule as recited in any one of the proceeding claims for use in the manufacture of a medicament for the prevention or treatment of a disease or condition ameliorated by the overexpression of fxn.

64. A transcription modulator molecule as recited in any one of the proceeding claims for use in the treatment of Friedreich's ataxia (FA).

65. A pharmaceutical composition comprising a transcription modulator molecule as recited in any one of the proceeding claims and a pharmaceutically acceptable carrier.

66. A method of modulation of the expression of fxn comprising contacting fxn with a transcription modulator molecule as recited in any one of claims 1-61.

67. A method of treatment of a disease caused by expression of a defective fxn comprising the administration of a therapeutically effective amount of a transcription modulator molecule as recited in any one of claims 1-61 to a patient in need thereof.

68. The method as recited in claim 67, wherein said disease is Friedreich's ataxia.

69. A method of treatment of a disease caused by expression of a defective fxn comprising the administration of:
a therapeutically effective amount of a transcription modulator molecule as recited in any one of claims 1-61; and
another therapeutic agent.

70. A method for achieving an effect in a patient comprising the administration of a therapeutically effective amount of a transcription modulator molecule as disclosed herein, or a salt thereof, to a patient, wherein the effect is chosen from muscular atrophy, ataxia, fasciculation, and dementia
